SEQUENCE LISTING (332 sequences)

SEQ ID NO: 1 - protein, 163 aa, African swine fever virus
MKYSWKNGGGDYWPIIIRHCCFYLVFSIAFVGYIVFAYYKNLHLNTAMKLLALLCILIWLSQPGLNRPLSIFYMKQNLPRTYTPPIRELEYWCTYGKHCDFCWECRNGICKNKVWDDMSSVQEHSYPMEHCMIHRQCKYIRDGPIFQVECTMQTSDATHLINA

SEQ ID NO: 2 - protein, 103 aa, African swine fever virus
MKVFLGLLLGYSTILILTYQSPTTQWCFYEISLKILNHHSMEKWRDKNWSIIIRYYCFYLVFSFAFAGCVAFAICKNLRLCTTMKLLMLLNILVLLSQPILNN

SEQ ID NO: 3 - protein, 375 aa, African swine fever virus
MNSLQVLTKKVLIENKAFSEYHEDDIFILQQLGLWWHNGPIGFCKQCKMVTSGSMSCSDVDSYELDRALVRAVKKNQTDLIKLFVLWGANINYGIICAKTERTKDLCIELGANPEFLDVGLYNMFVDLIKQQKVLLAIDIYYDNISILDSFDSHDFYVLIDFIYNCFILNLDEKEKMIKNTYVLKFWFKIAIEFNLIKPIRFLSKKFPHLDYWRLKTAVYLGNVDEIHHAYFQENIRLDPNDMMSLACMYPQNKLGIYYCFALGANINTALETLIGFINHEVNREITFFSNYGIWSNVHFCISLGANPYTKKIQETLLRQEKNVIMKLLFKKGLLSPHSILHKKILEPSEVRKIISTYEYTETFHSFSLLRDNLR

SEQ ID NO: 4 - protein, 356 aa, African swine fever virus
MVPSLQSFAKKVLASQHVSIDYHVILERCGLWWYKAPISLDCKHMLIKLPNFADGLDLNTALMLATKENNYQLIKMFTDWGADINYGLICANTPPIREFCWELGAKYQVDKKKIMHIFFKLIHPNTTSNNIILCLKFFNDNPFSAYVIIREIKSCIHWKLKNLAEDTNVLSNISDGDMLTIYCFIVALQDNLREAISYVYQHFKYLNTWWLTCALCYNKLFDLHNLYEKEKIRMDMDEMMRIACTKDNNFLTIYYCFILGANINLAMIASIRFYNMDNLFFCIDLGADAFEEAKALAEQQNYYLISHRLSLDIYSPDSSLLTLKEADPNKIYRLLKNYKSKSMLAYLNYDINDTSL

SEQ ID NO: 5 - protein, 353 aa, African swine fever virus
MLPSLQSLTKKVLAGQCVSVDHYHILKCCGLWWHNGPIMLHIRRNKLFIRSTCFSQGIELNIGLMKAVKENNHDLIKLFTEWGADINYGMICALTENTRDLCKELGAKEYLEREYILKIFFDTTRDKTSSNIIFCHEVFSNNPNLRIIDNLDLRGEIMWELRGLMEITFMLDHDDSFSTVLTKYWYAIAVDYDLKDAIRYFYQKYPRLHRWRLMCALFYNNVFDLHELYEIERVRMDIDEMMHIACIQDYSYSAIYYCFIMGANINQAMLVSIQNYNLGNLFFCIDLGANAFEEGKALAEQKENYLIAHALSLKHYNPVISLLSNVMDPEKINYMLKNYHSINMGIFLDYEQR

SEQ ID NO: 6 - protein, 350 aa, African swine fever virus
MLPSLQSLTKKVLAGQCVPTNQHYLLKYYDLWWYNAPITFDHNLRLIKSSGIKEGLDLNTALVKAVRENNYSLIKLFTEWGADINYGLVSVNTEHTRDLCQELGAKEILNEEEILQIFIDLKFHKTSSNIILCHEVFSNNPILQKVNNLKLRIEIFWELRELIEKTDLLNNEFLLSTLLLKYWYAIAVRYSLKEAIQYFYQKYTHMNTWRLTCALCFNNVFDLHEAYEKDKIHMDIEEMMRIACIKDHNLSTMYYCYMLGANINQAMLTSIQYYNIENMFFCMDLGADVFEEGTTALGEGYELIKNILSLKIYSPTTIPLPKSTDPEIIDHALKNYFSKNMMIFLSYDLR

SEQ ID NO: 7 - protein, 353 aa, African swine fever virus
MSLPLSLQTLVKKTVASQCLSIDEHCILKYCGLWWHDAPLKLCMDRGRIQIKSGFLGEDIDLRVALIIAVKENNYSLIKLFTEWGANINYSLLSINTKHIRELCRQLGAKETLEDNDIFRIFTRIMHNKTSGSIILCHEIFMNNPMLENKFVIQLRGLIYKRLWGLIEIKETDELNDLLVKYWYAKAVQYVCKNAICFLDEKYTDLNEWRLKCLLYYNKIYELHEMYHKKKVQIDVHDMICLACAKDNNLLTIYYCYALGGNINQAMLTSVQYYNVGNIFFCIDLGGNAFEEGRAIAEQKGYNFLSHSLTLDIYSSDASLPLNLKDPEKISSLLKDYKSKNLSIIWEYSHNIL

SEQ ID NO: 8 - protein, 357 aa, African swine fever virus
MLSLQTLAKKVVACNYLSSDYDYTLQRFGLWWDLGPIHLCNNCKQVFSYKHLQCFSEDDLCLEAALVKAVKSDNLELIRLFVDWGANPEYGLIRVPAVYLKRLCAELGGLTPVSEPRLLEILKEVANLKSCAGVLLGYDMFCHNPLLETVTRTTLDTVTYTCSNIPLTGDTAHLLLTKFWFALALRHNFTKAIHYFYKRHKNQLYWRVACSLYFNNIFDIHELCREKEICISPNLMMKFACLREKNYAAIYYCHRLGASLDYGMNLSIYNNNTLNMFFCIDLGAADFDRAQLIAHKAYMYNLSNIFLVKQLFSRDVTLVLDVTEPQEIYDMLKTYTSKNMKRAEEYLTAHPEIIVID

SEQ ID NO: 9 - protein, 531 aa, African swine fever virus
MFSLQNLCRKTLPDCKLPEFFDDYILQLLGLYWENHGTIQRAGNNCVLIQQHTLIPVNEALRIAASEENYEIVGLLLAWEGNLYYAIIGALEGNRYNLIRKYDDQIKDHHDILPFIDDPIIFHKCHIMRRCFFDCILYQAVKYSKFRVLLYFKYTLEDDLPLVHLLIEKACEDHNYEVIKWIYENLHVCHIIDTFDCAIAHKDLRLYCLGYTFIYNRIVPYKYHHLDILILSSLQLLHKVAAKGYLDFILETLKYDHNIDNLDVILTQAATYNHRKILTYFIPQSTYAQIEQCLFVAIKTKSSKKTLNLLLSHLNLSIKLIQKISQYVATFNSTNIIGILSMKRKKKIYLDIILTKFVKNAIFNKFVVRCMERFSINPERIVKMAARINKMMLVKKISEHVWKNHAARLKHLKHAVHTMKHKDGKNRLMNFIYEHCYYHMQGEEIFSLARFYAIHHAPKLFDVFYNCCILDTIRFKSLLLDCSHIIGKNAHDATNINIVNKYIGNLFAMGVLSKKEILQDYPSIYSKHYMP

SEQ ID NO: 10 - protein, 526 aa, African swine fever virus
MFSLQDLCRKHLFILPDVFGEHVLQRLGLYWRCHGSLQRIGDDHILIRRDLILSTNEALRMAGEEGNNEVVKLLLLWKGNLHYAVIGALQGDQYDLIHKYENQIGDFHFILPLIQDANTFEKCHALERFCGVSCLLKHATKYNMLPILQKYQEELSMRAYLHETLFELACLWQRYDVLKWIEQTMHVYDLKIMFNIAISKRDLTMYSLGYIFLFDRGNTEATLLTQHLEKTAAKGLLHFVLETLKYGGNIDTVLTQAVKYNHRKLLDYFLRQLPRKHIEKLLLLAVQEKASKKTLNLLLSHLNYSVKRIKKLLRYVIEYESTLVIKILLKKRVNLIDAMLEKMVRYFSATKVRTIMDELSISPERVIKMAIQKMRTDIVIHTSYVWEDDLERLTRLKNMVYTIKYEHGKKMLIKVMHGIYKNLLYGEREKVMFHLAKLYVAQNAATQFRDICKDCYKLDVARFKPRFKQLILDCLEIVTKKSCYSILEILEKHIISLFTMKVMTEEEKNLCLEILYKVIHYKTIQC

SEQ ID NO: 11 - protein, 254 aa, African swine fever virus
MQNKIPNFNLFFFFLYRMLEIVLATLLGDLQRLRVLTPQQRAVAFFRANTKELEDFLRSDGQSEEILSGPLLNRLLEPSCPLDILTGYHLFRQNPKAGQLRGLEVKMLERLYDANIYNILSRLRPKKVRNKAIELYWVFRAIHICHAPLVLDIVRYEEPDFAELAFICAAYFGEPQVMYLLYKYMPLTRAVLTDAIQISLESNNQVGICYAYLMGGSLKGLVSAPLRKRLRAKLRSQRKKKDVLSPHDFLLLLQ

SEQ ID NO: 12 - protein, 237 aa, African swine fever virus
MLEIVLATLLGDLQRLRVLTPQQRAVAFFRANTKELEDFLCSDGQSEEILSGPLLNRLLEPSGPLDILTGYHLFRQNPKAGQVRGLEVKMLERLYDANIYNILSRLRPEKVRNKAVELYWVFRAINMCHAPLVLDIVRYEEPDFAELAFICAAYFGEPQVMYLLYKYMPLTRAVLTDAIQISLESNSQVGICYAYLMGGSLKGLVRAPLRKRLRAKLRSQRKKKDVLPPHDFLLLLQ

SEQ ID NO: 13 - protein, 257 aa, African swine fever virus
MQNKIPNFNLFFFFLYKMLEIVLATLLGDLQKLKDLTPPQRAVAFFRANTKELEDFLYPDGQSEELLPGLLLNRLLEPSGSIDILTGYHLFRENPKAGRLRGLEVKMLERLYDANIYNMLARLRPELVRDKAIELYWLFRAILMCHSPLVLEIVRHETMDFAETAFICAAYFSEPQVMYALYKFIPISRAVLADAIQMCLESNSEAGICYAYLMGGSLKGKVPGSLRKRLRASPLRQERKKKNVLPPHEFLLMLHGI

SEQ ID NO: 14 - protein, 240 aa, African swine fever virus
MLEIVLATLLGDLQKLRDLTPMQRAVAFFRVNTKELEDFLYPDGQSEELLPGLLLNRLLEPSGPIDILTGYHLFRENPKAGRLRGLEVKLLERLYDANIYNMLAQIRPELVRIKAIELYWLFRAILMCHSPLVLEIVRHETMDFAELAFICAAYFSEPQVMYALYKFIPISRAVLADAIEMSLESNSETGICYAYLMGGSLKGKVPGPLRKRLRASPLRQERKKKNVLPPHEFLLMLHGI

SEQ ID NO: 15 - protein, 254 aa, African swine fever virus
MQNKIPNFNLFFFFLYRMLEIVLATLLGDLQRLRVLTPQQRAVAFFRANTKELEDFLCSDGQSEEILSGPLLNRLLEPSGPLDILTGYHLFRQNPKAGQLRGLEVKMLERLYDANIYNILSRLRPEKVRNKAIELYWVFRAIHICHAPLVLDIVRYEEPDFAELAFICAAYFGEPQVMYLLYKYMPLTRAVLTDAIRISLESNNQVGICYAYLMGGSLKGLVSAPLRKRLCAKLRSQRKKKDVLSPHDFLLLLQ

SEQ ID NO: 16 - protein, 237 aa, African swine fever virus
MLEIVLATLLGDLQRLRVLTPQQRAVAFFRANTKEVEDFLCSDGQSEEVLSGPLLNRLLEPSGPLDILTGYHLFRQNPKAGQVRGLEVKMLERLYDANIYNILSRLRPEKVRNKAVELYWVFRAINMCHAPLVLDIVRYEEPDFAELAFICAAYFGEPQVMYLLYKYMPLSRAVLTDAIQISLESNSQVGICYAYLMGGSLKGLVRAPLRKRLRAKLRSQRKKKDVLPPHDFLLLLQ

SEQ ID NO: 17 - protein, 237 aa, African swine fever virus
MLEIVLATLLGDLQRLRVLTPQQRAVAFFRANTKELEDFLCPDGQSEEVLSGSLLNRLLEPSGPLDILTGYHLFRQNPKAGQLRGLEVKMLERLYDANIYNILSRLRPEKVRNKAVELYWVFRAINMCHAPLVLDIVRNEELDFAELAFICAAYFGEPQVMYLLYKYMPLTRAVLTDAIQISLESNSQVGICYAYLMGGSLKGLVRAPLRKRLRAKLRSQRKKKDVLPPHDFLLLLQ

SEQ ID NO: 18 - protein, 254 aa, African swine fever virus
MQNKIPNFNLFFFFLYRMLEIVLATLLGDLQRLRVLTPQQRAVAFFRANTKELEDFLCSDGQSEEILSGPLLNRLLEPSGPLDILTGYHLFRQNPKAGQLRGLEVKMLERLYDANIYNILSRLRPEKVRNKAIELYWVFRAIHICHAPLVLDIVRYEEPDFAELAFICAAYFGEPQVMYLLYKYMPLTRAVLTDAIRISLESNNQVGICYAYLMGGSLKGLVSAPLRKRLRAKLRSQRKKKDVLSPHDFLLLLQ

SEQ ID NO: 19 - protein, 72 aa, African swine fever virus
MQNKIPNFNLFFFFLYRMLEIVLATLLGDLQRLRVLTPQQRAVAFFRANTKELEDFLRSDGQSEEILSGPPP

SEQ ID NO: 20 - protein, 153 aa, African swine fever virus
MYFKKKYIGLIDKNCEKKILDDSSTIKICYILIGILIGTNMITLIYNFIFWDNYIKCYRNNDKMFYCPNDWVGYNNICYYFSNGSFSKNYTAASNFCRQLNGTLANNDTNLLNLTKIYNNQSMYWVNNTVILRGDNKYSQKVNYTDLLFICGK

SEQ ID NO: 21 - protein, 402 aa, African swine fever virus
MIIIVIFLMCLKIVLNNIIIWSTLNQTVFLNNIFTINDTYGGLFWNTYYDNNRSNFTYCGIAGNYCSCCGHNISLYNTTNNCSLIIFPNNTEIFNRTYELVYLDKKINYTVKLLKSVDSPTITYNCTNSLITCKNNNGTNVNIYLIINNTIVNDTNGDILNYYWNGNNNFTATCMINNTISSLNETENINCTNPILKYQNYLSTLFYIIIFIVSGLIIGIFISIISVLSIRRKRKKHVEEIESPPPSESNEEDISHDDTTSIHEPSPREPLLPKPYSRYQYNTPIYYMRPSTQPLNPFPLPKPCPPPKPCPPPKPCPPPKPCPPPKPCSPPKPCRPPKPCPPPKPCPPPKPCPPPKPCPPSKPCPSPESYSPPKPLPSIPLLPNIPPLSTQNISLIHVDRII

SEQ ID NO: 22 - protein, 392 aa, African swine fever virus
MIIILIFLICLKIVLNNNIIIWHTLNETIFLNNSITFTDNLGGLFWNTYYNNNHSILTSCGIYQNYCSCTNNYSTSLYNITNNCGLIIFPNNTKRFNTTYELVYLNNSTKYTINLLVPVTPPNITYNCTTSVITCKKNDGTNTNMFLKINDTIVKSSNESVFEYSWNSTDLNNNFTAICIINNTINSSNDTQTIECPNILLNNYLDFFQVTSYMFYMIIFIVTGIIASIFISIITFLSLRKRKKHVEEIESPPPSESNEEDISHDDTTSIHEPSPREPLLPKPYSRYQYNTPIYYMRPSTQPLNPFPLPKPCPPPKPCPPPKPCPPPKPCPPPKPCRPPKPCPPPKPCPPPKPCPSPETYSPPKPLPNIPLLPNIPPLSTQNISLIHVDRII

SEQ ID NO: 23 - protein, 389 aa, African swine fever virus
MIIILIFLIYLKLVLNNNIIIWHPTLNETIFLNVTTIGNISGLFWNTYYNNNRSELTKCGIYNNYCSCDSLNTSLYNINNCSLVIFPNNTKIFNTIYELIYSGNQTNYTIKLLESTTPPNITYNCTTSVITCKKNNGTNTNIFLTINNTIVNSTNEDVIYYYWNRSEFNNFTATCMINNTINSSNISEIIDCTNTLLLNSYLDFFQVAGYMFYMIIFIVTGITVSIFISIITFLSLRKRKKHVEEIESLPPESNEEECQHDDDTTSIHEPSPREPLLPKPYSRYQYNTPIYYMRPSTQPLNPFPLPKPCPPPKPCPPPKPCPPPKPCPPPKPCPPPKPCPPPESCSSPESCSSPESYSPPKPLPSIPLLPNIPPLSTQNISLIHVDRII

SEQ ID NO: 24 - protein, 360 aa, African swine fever virus
MIILIFLIFSNIVLSIDYWVSFNKTIILDSNITNDNNDINGVSWNFFNNSFNTLATCGKAGNFCECSNYSTSIYNITNNCSLTIFPHNDVFDTTYQVVWNQIINYTIKLLTPATPPNITYNCTNFLITCKKNNGTNTNIYLNINDTFVKYTNESILEYNWNNSNINNFTATCIINNTISTSNETTLINCTYLTLSSNYFYTFFKLYYIPLSIIIGITISILLISIITFLSLRKRKKHVEEIESPPPESNEEEQCQHDDTTSIHEPSPREPLLPKPYSRYQYNTPIYYMRPSTQPLNPFPLPKPCPPPKPCPPPKPCPPPKPCPSAESYSPPKPLPSIPLLPNIPPLSTQNISLIHVDRII

SEQ ID NO: 25 - protein, 375 aa, African swine fever virus
MIIILIFLIIPNIVLSIDYWVSFNKTIILDSNITNDNNDINGVSWNFLNNSLNTLATCGKAGNFCECSNYSTSLYNIAHNCSLTIFPHNDVFGTPYQVVWNQIINYTIKLLTPVTPPNITYNCTNFLITCKKNNGTNTIIYFNINDTNVKYTNESILEYNWNNSNFNNFTATCIINNTINSSNDTQTIDCINTLLSSYLDFFQVASYMFYMIIFIATGIIASIFISIITFLSLRKRKKHVEEIESPSPSESNEEEQCQHDDTTSIHEPSPREPLLPKPYSRYQYNTPIYYMRPLTQPLNPSPLPKLCPPPKPCPPPKPCPPPKPCPPPKPCPSSESCSPPESYSLPKPLPNIPLLPNIPPLSTQNISLIHVDRII

SEQ ID NO: 26 - protein, 392 aa, African swine fever virus
MIIKLIFLICFKIVLSIDNKTKFNETLILDNIDYWVKFNDTITLDSNITSEIEAVSWNFYNNTFNLLAICGKASNFCSCSNYSTSFFNITNNCSLTIFLIDETLFNTTYQIVYSTNIINYKINLLIPVTPPIISYNCANCSINCKKSNGTNTNIFLSINDTIVTYTNESILNYDYNCSILNNNFTVTCIINNTISTSNTTEFINCTNILLSSYLDFFQVTSYIFYMIIFIVTGITVSILISIITFLFIRKRKHVEEIESPPPESNEEEQQCHHDTTSIHEPSPREPLLPKPYSRYQYNTPIYYMRPSTQQLFKSYSLPKPCPPPKPCPPPKPCPPPKPCPPSKPCPPPEPYSPPKPCPPPKPYPSLPSIPLPPDIPPLSTQNISLIHVDRII

SEQ ID NO: 27 - protein, 385 aa, African swine fever virus
MIIKLIFLISFKMILGIDYWVSLNNTIILDSNITINNITNTTNNPTLNGIFWNIYNNSYNNTFNLLTTCGNTYNICSCSNNYNTILFNYNTTNNCSLIISPHDEKIFDTMFQIIYLTNKINYTIRWLQPVDPPNISFNDSNSLIKCEKNNGTNTEIYLYLNDTFINNTNENDLKYYWNCSELNYNITATCIINNTLNSANTTKVINCTNLLLKSDQNYFLKNIHTLFYIIIFIVTGIIISIFIAIITFLSLRKRKKHVEEIESPSPESNEEEEQQHHHDTTSIHEPSPREPLLPKPYSRYQYNTPIYYMRPSTQQLFKSYSLPKPCPPPKPCPPPKPCPPPKPCPSPESYPSPEPYPLLPNIPLPPNIPPLSTQNISLIHVDRII

SEQ ID NO: 28 - protein, 382 aa, African swine fever virus
MIIKVIFLVFFKMVLSNIIIWSTLNNTIYLNINYSDSNLYTGLFWNKYYNNTRNNFTTCVTSNSSYCTCKGHNTSLYNITNNCSLIIFPNNTKIFNTTYELVYLNNKINYTIQMVQPVDPPIIIYNDTNNSLITCEKNNGTNTEIYLYLNDTFINNTNENSIKYYWNCSELNHNITTTCIINNTLNSANTTKVINCTNLLLKSDQNYFLKNIHTLFYIIIFIVGGTLISIIISIITFLSLRKRKKHVEEIESPPPSESNEEEHCQHDDTTSIHEPSPREPLLPKPYSRYQYNTPIYYMRPSTQPLNPFPLPKPCPPPKPCPPPKPCPPPKPCPPPKPCPPPKPCPSPESHSPPKPLPSIPLLPNIPPLSTQNISLIHVDRII

SEQ ID NO: 29 - protein, 404 aa, African swine fever virus
MFITLIFLSYINIVLSNNYWARLNETITLNSNITNDTNNELGIFWNSYNNTYYNNTFNNIAICGKKGIFCECNINYNTSISNTSISNTSIYNVTNNCSLTIFLYDDNIFKTYQLVYQNYKINYTINLLLPVTSPNITYNCTNSLITCEKNDGTNTNMFLSINNITINHTNQDILTYYWNNSEFNNFTATCMINNTLNSANTTKVINCTNPLLNSYQNYFLENIHTLFYIIIFIVSGLIASIFISIITFLSLRKRKKHVEEIESPPPESNEEEQCQHDDTTSIHEPSPREPLLPKPYSRYQYNTPIYYMRPSTQPLNPFPLPNPCPPPKPCPPPKPCPPPKPCPPPKPCPPPKPCPPPKPCPPPKPCSSPESYSPPKPLPSIPLLPNIPPLSTQNISLIHVDRII

SEQ ID NO: 30 - protein, 381 aa, African swine fever virus
MIIKLIFLICFKIVLSINYWVRYNDTVTLNSNINSETEGIFWNFYNNTFNTIATCGKKNNVCECSNYDKSLYNITNNCSLTIFPNNTKIFNTTYQLVYSRNRINYTINLLLPVTSPIITYNCTQSLITCEKTNGTNIHLFLNLNDTINEYTNKSFLNYYWNSSELNNIFLATCIINNTLNSANTTKVINCTNPLLKSYQNYFLENIHTLFYMIIFIVSGITISIFISIITFLSLRKRKKHVEEIESPPPESNEEEQCQHDDTTSIHEPSPREPLLPKPYSRYQYNTPIYYMRPSTQPLNPFPLPKPCPPPKPCPPPKPCPPPKPCPPPKPCPPPKPCPPPKPCPPPESYSPPKPLPSIPLLPNIPPLSTQNISLIHVDRII

SEQ ID NO: 31 - protein, 402 aa, African swine fever virus
MIIIVIFLMCLKIVLNNIIIWSTLNQTVFLNNIFTINDTYGGLFWNTYYDNNRSNFTYCGIAGNYCSCCGHNISLYNTTNNCSLIIFPNNTEIFNRTYRLVYLDKKINYTVKLLKSVDSPTITYNCTNSLITCKNNNGTNVNIYLIINNTIVNDTNGDILNYYWNGNNNFTATCMINNTISSLNETENINCTNPILKYQNYLSTLFYIIIFIVSGLIIGIFISIISVLSIRRKRKKHVEEIESPPPSESNEEDISHDDTTSIHEPSPREPLLPKPYSRYQYNTPIYYMRPSTQPLNPFPLPKPCPPPKPCPPPKPCPPPKPCPPPKPCSPPKPCRPPKPCPPPKPCPPPKPCPPPKPCPPSKPCPSPESYSPPKPLPSIPLLPNIPPLSTQNISLIHVDRII

SEQ ID NO: 32 - protein, 402 aa, African swine fever virus
MIIIVIFLMCLKIVLNNIIIWSTLNQTVFLNNIFTINDTYGGLFWNTYYDNNRSNFTYCGIAGNYCSCCGHNISLYNTTNNCSLIIFPNNTEIFNRTYELVDLDKKINYTVKLLKSVDSPTITYNCTNSLITCKNNNGTNVNIYLIINNTIVNDTNGDILNYYWNGNNNFTATCMINNTISSLNETENINCTNPILKYQNYLSTLFYIIIFIVSGLIIGIFISIISVLSIRRKRKKHVEEIESPPPSESNEEDISHDDTTSIHEPSPREPLLPKPYSRYQYNTPIYYMRPSTQPLNPFPLPKPCPPPKPCPPPKPCPPPKPCPPPKPCSPPKPCRPPKPCPPPKPCPPPKPCPPPKPCPPSKPCPSPESYSPPKPLPSIPLLPNIPPLSTQNISLIHVDRII

SEQ ID NO: 33 - protein, 360 aa, African swine fever virus
MIILIFLIFSNIVLSIDYWVSFNKTIILDSNITNDNNDINGVSWNFFNNSFNTLATCGKAGNFCECSNYSTSIYNITNNCSLTIFPHNDVFDTTYRVVWNQIINYTIKLLTPATPPNITYNCTNFLITCKKNNGTNTNIYLNINDTFVKYTNESILEYNWNNSNINNFTATCIINNTISTSNETTLINCTYLTLSSNYFYTFFKLYYIPLSIIIGITISILLISIITFLSLRKRKKHVEEIESPPPESNEEEQCQHDDTTSIHEPSPREPLLPKPYSRYQYNTPIYYMRPSTQPLNPFPLPKPCPPPKPCPPPKPCPPPKPCPSAESYSPPKPLPSIPLLPNIPPLSTQNISLIHVDRII

SEQ ID NO: 34 - protein, 198 aa, African swine fever virus
MIIIVIFLMCLKIVLNNIIIWSTLNQTVFLNNIFTINDTYGGLFWNTYYDNNRSNFTYCGIAGNYCSCCGHNISLYNTTNNCSLIIFPNNTEIFNRTYELVYLDKKINYTVKLLKSVDSPTITYNCTNSLITCKNNNGTNVNIYLIINNTIVNDTNGDILNYYWNGNNNFTATCMINNTISSLNETENINCTNPILKY

SEQ ID NO: 35 - DNA, 357 bp, African swine fever virus
atgttggtaatctttttgggaattcttggccttctggccagccaggtctcaagtcaaccagatggacaacttcgtccaacagaggatcctccagaagaagaacttaaatattggtgcacctacatggaaagttgccagttttgttgggactgccaagatggcaattgtataaacaaagtagatgggtcagtcatttataaaaatgagtttgtgcgaccatgttcagtttcccgctggatgaataaatgtatgtatgatttaaataagggtatctatcatacaatgaattgttctcagccacagtcttggaatccctacaaatacttcaggaaggagtggaaaaaagatgaactctag

SEQ ID NO: 36 - DNA, 357 bp, African swine fever virus
atgttggtgatctttttgggaattcttggccttctggccagccaggtctcaagtcagccagttggacaacttcgtccaacagaggaccctccagagaatgaactcgaatattggtgcacctacatggaaagttgccagttttgctgggactgtcaagatggcaattgtataaacaaagtagatgggtcagtcatttataaaaatgagtatgtgcaaccatgttcagtttcccgctggctaaataaatgtatgtatgatttagataagggtatctatcatacaatgaactgttctcagccacggtcttggaatccctacaaatgtttcaggaaggagtggaaaaaagatgaactctag

SEQ ID NO: 37 - DNA, 369 bp, African swine fever virus
atgttggtgatctttttgggaattcttggccttatggccagccaggtcttagggctaccatctaatcaaccaactggacaacttcgtccaacagaggatcctccagaggaagaactcgaatactggtgtgcctacatggaaagttgccagttttgctgggactgccaagatggcaattgcataaacaaaatagatgggtcagtcatttataaaaatgagtttgtgcgaccatgttcagtttcccgctggatggataaatgtatgtatgatttaaataagggtatctatcatacaatgaattgttctcagccacagtcttggaatccctacaaatacttcaggaaggagtggaaaaaagatgaactctag

SEQ ID NO: 38 - DNA, 357 bp, African swine fever virus
atgttggtgacctttttgggaattcttggccttctggccagccaggtctcaagtcagctcgttggacaacttcgtccaacagaggatcctccagaggaagaactcgaatactggtgcgcctacatggaaagttgccagttttgctgggactgccaagatggcaattgtataaacaaaatagatgggtcagtcatttataaaaatgagtatgtgcgaccatgttcagtttcccgttcgatggataaatgtatgtatgatttaaataagggtatctaccatagcatgagctgttctgacccaaaggcctggaatccctacaaatacttcaggaaggaatggaaaaaagatgaactctag

SEQ ID NO: 39 - DNA, 357 bp, African swine fever virus
atgttggtgatctttttgggaattcttggccttctggccagccaggtttcaagtcaactcgttggacaacttcgaccaacagaggatcctccagaggaagaactcgaatactggtgcgcctacatggaaagttgccaattttgctgggactgccaagatggcacttgtataaacaaaatagatgggtcagtaatttacaagaatgagtatgtgaaagcatgtctggtttcccgttggctggataaatgtatgtatgatttagataaaggtatctaccataccatgaattgttctcagccatggtcttggaatccttacaaatacttcaggaaggaatggaaaaaagatgaactctag

SEQ ID NO: 40 - DNA, 357 bp, African swine fever virus
atgttggtgatcttcttgggaattcttggccttctggccagccaggtttcaagtcagctcgttggacaacttcgtccaacagaggatcctccagaggaagaactcgaatactggtgcgcctacatggaaagttgccaattttgctgggactgccaagatggcacttgtataaacaaaatagatgggtcagtaatttacaagaatgagtatgtgaaatcatgtctggtttcccgttggctggataaatgtatgtatgatttagataaaggtatctaccataccatgaattgttctcagccatggtcttggaatccttacaaatacttcaggaaggaatggaaaaaagatgaactctag

SEQ ID NO: 41 - DNA, 357 bp, African swine fever virus
atgttggtgatcttcttgggaatttttggccttctggccagccaggtttcaagtcagctcgttggacaacttcgtccaacagaggatcctccagaggaagaactcgaatactggtgtgcctacatggaaagttgccaattttgctgggactgccaagatggcacttgtataaacaaaatagatgggtcagtaatttacaagaatgagtatgtgaaatcatgtctggtttcccgttggctggataaatgtatgtatgatttagataagggtatctaccataccatgaattgttctcagccatggtcttggaatccttacaaatacttcaggaaggaatggaaaaaagatgaactctag

SEQ ID NO: 42 - DNA, 357 bp, African swine fever virus; identical to SEQ ID NO: 40

SEQ ID NO: 43 - DNA, 357 bp, African swine fever virus
atgttggtgatcttcttgggaattcttggccttctggccagccaggtttcaagtcagctcgttggacaacttcgaccaacagaggatcctccagaggaagaactcgaatactggtgcgcctacatggaaagttgccaattttgctgggactgccaagatggcacttgtataaacaaaatagatgggtcagtaatttacaagaatgagtatgtgaaatcatgtctggtttcccgttggctggataaatgtatgtatgatttagataaaggtatctaccataccatgaattgttctcagccatggtcttggaatccttacaaatacttcaggaaggaatggaaaaaagatgaactctag

SEQ ID NO: 44 - DNA, 375 bp, African swine fever virus
atgaaggtgctgattctagtactactgggggtcgttatccttcaggctgctcctatacgtaaattagaagacctactgccaacccgtaatcctccccaaaatgagctagtttactggtgcacctacgcaaaccaatgtgacttttgctgggaatgtgtacacggaatctgccgaaacagaattcaagcagattggcctgtgattcaccaaaatgactggattataaattgcaccgtttcccgctggaatggtatatgtaggtattatgaaggacccagagtccatatagatcatgaaatggactgtgcaaatccaacatcccatacttatccacacattgaatacatgaagatctatgaaagagatgacctatga

SEQ ID NO: 45 - DNA, 375 bp, African swine fever virus
atgaaggtgctgattctagtactactgggggtcgttatccttcaggctgctcctatacgtaaattagaagacctactgccaacccgtaatcctccccaaaatgagttagtttattggtgcacctacgcaaaccaatgtgatttttgctgggaatgtatacacggaatctgccgaaacagaattcaagcagattggcctgtgattcaccaaaatgactggattataaattgcaccgtttcccgctggaatggtatatgtaggtattatgaaggacccagaggccatatagatcatgaaatggactgtgcaaatccaacataccatacttatccacacattgaatacatgaagatttatgtaagagatgacctatga

SEQ ID NO: 46 - DNA, 375 bp, African swine fever virus
atgaaggtgctgattctagtactgctggcggtggttatccttcaagccgctcctatacgtaaattagaagatctactaccaacccgttatcctcctgaccatgagctagtttattggtgcacctacgcaaaccaatgtgacttttgctgggaatgcgtacacggtatctgccgaaataggattcaagcagattggccggttattcaccaaaatgactggattataaattgcacggtttcccgctggaatggtatatgtagttattatgaaggacccagaaaccatacagatcatcaaatggactgtgcaaatccaacatcccatacctatccacatagagaatacatgaaaatttatgaaagagatgacttataa

SEQ ID NO: 47 - DNA, 381 bp, African swine fever virus
atgaaggtgctgattctagtactactggggggggggggtattattcttcaggccgctcctatacgtaaattagaagatctactaccaacccgttatcctcctgaacatgagctggtttattggtgcacctacgcaaaccaatgtgacttttgctgggaatgcgtccacggtatttgccgaaacaggattcaagcagattggccggttattcatcaaaatgactggattataaattgcacggtttcccgctggaatggccaatgtcattattatgaaggttctcaacaatatctacatcatgaaatggactgtacaaatccaacatcccatacctatccacatacagaatacatgaagatctatgaaagagatgacttatga

SEQ ID NO: 48 - DNA, 375 bp, African swine fever virus
atgaaggtgctaattctagtactactgggggtggttatccttcaggccgctcctatacgtaaattagaagatctactaccaacccgttatcctcctgaacatgagctagtttattggtgcacttacgcaaaccaatgtgacttttgctgggaatgcgtccacggtatctgccgaaacaggattcaagcagattggccagttattcatcaaaatgactggattataaattgcacggtttcccgctggaatggtatatgtagttattatgaaggacccagaaaccatacagatcatcaaatggactgtgcaaatccaacatcccatacttatccacatagagaatacatgaagatctatgaaagagatgacttatga

SEQ ID NO: 49 - DNA, 375 bp, African swine fever virus
atgaaggtgctaattctagtactactgggggtggttattcttcaggccgctcctatacgtaaattagaagatctactaccaacccgttatcctcctgaacatgagctagtttattggtgcacttacgcaaaccaatgtgacttttgctgggaatgcgtccacggtatctgccgaaacaggattcaagcagattggccagttattcaccaaaatgactggattataaattgcacagtttctcgctggaatggtatatgtagttattatgaaggacccagaaaccatacagatcatcaaatggactgtgcaaatccaacatcccatacttatccacatagagaatacatgaagatctatgaaagagatgacttatga

SEQ ID NO: 50 - DNA, 375 bp, African swine fever virus
atgaaggtgctaattctagtactactgggggtggttattcttcaggccgctcctatacgtaaagtagaaaatctactgccaacccgtaatcctccccaaaatgaattagtttattggtgcacctatgcaaaccaatgtgacttttgctgggaatgcattcacggcatctgccgaaacaggattcaagcagattggccagttattcatcaaaatgactggattataaattgcacggtttcccgctggaatggccaatgtcattattatgaaggttctcaacaatatctgcatcatgaaatggactgtataaatccaacatcccatacctatccacatacagaatacatgaagatctatgaaagagatgacttatga

SEQ ID NO: 51 - DNA, 1128 bp, African swine fever virus
ttaccgcaaattgtctctcagcgaagaaaatgaatgaaacgtttctgtatattcataggttgaaattattttacgcacttcactaggttctaatattttcttatgaagtattgaatgggggcttaaaagtcctttcttaaaaagaagtttcatcataacattcttttcttgtctaagaagagtttcttgtattttttttgtataaggattggcacccaaacttatacaaaaatgtacattactccaaataccataatttgaaaagaaagttatttccctatttacttcatgattaatgaaacctatcaacgtctctaaggccgtattgatatttgcgcctaaggcaaaacaatagtatatacccaatttattttgagggtacatacaagcaagcgacatcatgtcatttggatctaaacgtatattttcctgaaaatatgcatgatggatttcatcaacattacctaagtatacagccgtttttaaacgccaataatctaggtgaggaaatttcttactaagaaaacgaataggttttataagattaaactctatggcgatcttaaaccaaaattttaatacatatgtattttttatcattttttctttttcatctaaatttaagataaaacgattgtaaataaagtctatcaacacgtaaaaatcatggctatcaaaactgtcgagaatcgaaatattgtcataataaatatctatagctaataagaccttttgttgtttaattagatcaacaaacatattatacaaccctacatctaaaaattttggatcagctcctagttgaatacacagaactttcgtcctttccgtcttggcacatatgatgccataattaatgttggcaccccataaaacaaataacttgattagatcagtctggtttttcttcacagccctcaccaaggctctgtcaagctcatagctgtcaacatcagaacatgacatagagccactggttaccattttacattgtttacaaaaacctatgggtccgttttcccaccataatccaagctgctgtaaaataaaaatatcatcctcatgataatttgaaaaagccttgttttctatcaagactttttttgtaagaacctgtaaagaattcat

SEQ ID NO: 52 - DNA, 1128 bp, African swine fever virus; identical to SEQ ID NO: 51

SEQ ID NO: 53 - DNA, 1128 bp, African swine fever virus
ttaccgcaaattgtctctcagtaaagaaaatgaatgaaacgtttctgtatattcataggttgaaattattttacgcacttcaccaggttctaatatttttttatgaagtattgaatgggggcttaaaagtcctttcttaaaaagaagtttcattataacattcttttcttgtctaagaagagtttcttgtattttttttgtataaggattggcacccaaacttatacaaaaatgtacattactccaaataccataatttgaaaagaaagttatttccctatttacttcatgattaatgaaacctatcaacgtctctaaggcagtattgatatttgcacctaaggcaaaacaatagtatatacctaatttattctgagggtacatacaagcaagcggcatcatgtcatttggatctaaacgtatattttcctgaaaatatgcatgatggatttcatcaacattacctaagtatacagccgtttttaaacgccaataatctaggtaaggaaatttcttactaagaaaacgaataggttttataagattaaactctatggcgatcttaaaccaaaattttaatacatatgtattttttatcattttttctttttcatctaaatttaagataaaacaattgtaaataaagtctatcaacacgtaaaaatcatggctatcaaacctgtcgagaatcgaaatattatcataataaatatctatagctaataagaccttttgttgtttaattagatcaacaaacatattatacaaccctacatctaaaaattttggatcagctcctagttgaatacacagagctttcatcctttctgtcttggcgcatatgatgccataattaatgttggcaccccataaaacaaataacttgattagatcagtctggtttttcttcacagccctcaccaaggctctgtcaagctcatagctgtcaacgtcagagcatgacatagcgccactagttaccattttacattgtttacaaaaacctatgggaccgttttcccaccatagtccaagctgctgtaaaataaaaatatcatcttcatgatattttgaaaaagccttgttttctatcaagactttttttgtaagaacctgtaaagaattcat

SEQ ID NO: 54 - DNA, 1128 bp, African swine fever virus
ttaccgcaaattgtctctcagcaaagaaaatgaatgaaacgtttccgtatattcataggttgaaattattttacgcacttcactaggttctaatattttcttatgaagtattgaatgggggcttaaaagtcctttcttaaaaagaagtttcattataacattcttttcttgtctaagaagagtttcttgtattttttttgtataaggattggcacccaaacttatacaaaaatgtacattactccaaataccataatttgaaaagaaagttatttccctatttacttcatgattaatgaaacctatcaacgtctctaaggcagtattgatatttgcgcctaaggcaaaacaatagtatatacctaatttattctgagggtacatacaagcaagcgacatcatgtcatttggatctaaacgtatattttcctgaaaatatgcatgatggatttcatcaacattacctaagtatacagccgtttttaaacgccaataatctagatgaggaaatttcttactaagaaaacgaataggttttataagattaaactctatggcgatcttaaaccaaaattttaatacatatgtattttttatcattttttctttttcatctaaatttaagataaaacaattgtaaataaagtctatcaatacgtaaaaatcatggctatcaaaactgtcgagaatcgaaatattatcataataaatatctatagctaataagaccttttgttgtttaattagatcaacaaacatattatacaatcctacatctaaaaattctggattggctcctagttcaatacacaggtctttcgtcctttccgtcttggcacatatgatgccataattaatgttggcaccccataaaacaaacaacttgattagatcagtctggtttttcttcacagcccttaccaaggctctgtcaagctcatagctgtcaacgtcagagcatgacatagagccactggttaccattttacattgtttacaaaaacctatggggccattatgccaccacaatccaagctgctgtaaaataaaaatatcatcctcatgatattctgaaaaagccttgttttctatcaagactttttttgtaagaacctgtaaagaattcat

SEQ ID NO: 55 - DNA, 1128 bp, African swine fever virus; identical to SEQ ID NO: 54

SEQ ID NO: 56 - DNA, 1128 bp, African swine fever virus
ttaccgcaaattgtctctcagcaaagaaaatgaatgaaacgtttccgtatattcgtaggttgaaattattttacgcacttcactaggttctaatattttcttatgaagtattgaatgggggcttaaaagtcctttcctaaaaagaagtttcattataacattcttttcttgtctaagaagagtttcttgtattttttttgtataaggattggcacccaaacttatacaaaaatgtacattactccaaataccataatttgaaaagaaagttatttccccatttacttcatgattaatgtaacgtatcaatgtctctaaggcagtattgatatttgcgcctaaggcaaaacaatagtatatacctaatttattctgagggtacatacaagcaagcggcatcatgtcatttggatctaaacgtatattttcctgaaaatatgcatgatggatttcatcaacattacctaagtatacagccgtttttaaacgccaatcatctaggtgaggaaatttcttactaagaaaacgaataggttttataagattaaactctatggcgatcttaaaccaaaattttaatacatatgtattttttatcattttttctttttcatctaaatttaagataaaacaattgtaaataaagtctatcaacacgtaaaaatcatgactatcaaacctgtcgagaatcgaaatattatcataataaatatctatagctaataagaccttttgttgtttaattagatcaacaaacaaattatacaaccctacatctaaaaattttggatcagctcctagttggatacatagaactttcgtcctttccgtcttggcacatatgatgccataattaatgttggcaccccataaaacaaataacttgattagatcagtctggtttttcttcacagccctcaccaaggctctgtcaagctcatagctgtcaacgtcagagcatgacatagagccactggttaccattttacattgtttacaaaaacctatggggccattatgccaccatagtccaagctgctgtaaaataaaaatatcatcttcatgataatttgaaaaagccttgttttctatcaagactttttttgtaagaacctgtaaagaattcat

SEQ ID NO: 57 - DNA, 1128 bp, African swine fever virus
ttactgcaaattgtctctcagcaaagaaaatgaatgaaacgtttctgtatattcataggttgaaattattttacgcacttcattaggttctattattttcttatgaagtattgaatgggggcttaaaagtcctttcttaaaaagaagtttcattataacattcttttcttgtctaagaagagtttcttgtattttttttgtataaggattggcacccaaacttatacaaaaatgtacattactccaaataccataatttgaaaagaaagttatttccctatttccttcatgattaatgaaacctatcaacgtctctaaggcagtattgatatttgcgcctaaggcaaaacaatagtatatacctaatttattctgagggtacatacaagcaagcgacatcatgtcatttggatctaaacgtatattttcttgaaaatatgcatgatggatttcatcaacattacctaagtatacagccgtttttaaacgccaatcatctaggtgaggaaatttcttactaagaaaacgaataggttttataagattaaactgtatggcgaacttaaaccaaaattttaatacaagtgtattttttgtcatttcttctttttcatctaagtataggataaaacgattgtacataaagtctatcaacacgtaaaaatcatggctatcaaaactgtcgagaatcgaaacattatcataataaatatctatagctaataagaccttttgttgtttaagtagatcaacaaacatattatacaaccctacatctaaaaattttggatcagctcctagttgaatacacagaactttcgtcctttccgtcttggcacatatgatgccataattaatgttggcaccccataaaataaataacttgattagatcagtctggtttttcttcacagccctcaccaaggctctgtcaagctcatagctgtcaacgtcagagcatgacatagagccactggttaccattttacattgtttacaaaaacctatggggccattatgccaccatagtccaagctgctgtaaaataaaaatatcatcttcatgatattctgaaaaagccttgttttctatcaagactttttttgtaagaacctgtaaagaattcat

SEQ ID NO: 58 - DNA, 1128 bp, African swine fever virus
ttatcgtaaattgtctcttagcgaagaaaatgaatgaaccgtttccgtatattgataggttgaaattattttacgcacttcactaggttctaatattttcttatgaagtattgaatgggggcttaaaagtcctttcttaaaaagtagtttcattataacattcttttcttctctaagaagagtttcttgtattttttttgtataaggattggcacccaaacttatacaaaaatgtacattactccaaataccataatttgaaaagaaagttatttccctatttacttcatgattaatgaaacgtatcaacgtctctaaggcagtattgatatttgcccctaaggcaaaacaatagtatatacctaacttattttgagggtacatacaagcaagcgacatcatgttatttggatctaaacgtatattttcctgaaaatatgcatgatggatttcatcaacattacctaagtatacagccgtttttaaacgccaatgatctaggtgaggaaatttcttactaagaaaacgaataggttttataagattaaactctatcgcgaacttataccaaaattttaatacaagtgtatttcttgtcatttcttctttttcatctaaatttaagataaaacgattgtaaataaagtctatcaacgtgtaaaaatcatggtcatcaaaactatcgagaatcgaaatattatcataataaatatctatagctaataagactttttgttgtttaagtagatcaacaaacatattatacaaccctacatctaaaaattttggatcagctcctagttgaagacacagaacttttgtcctttccgtcttggcacacataatgccataattaacgttggcaccccataaaacaaataacttgattagatcagtctggtttttcttcacagccgtcaccaaggctctgtcaagctcatagctgtcaacatcagagcatgacatagagccactggttaccattttacattgtttacaaaaacctatggggccatcatgccaccacaaccctagctgctgtaaaataaaaatatcatcctcatgataatttgaaaaggccttggtttctatcaagactttttttgtaagaacctgtaaagaattcat

SEQ ID NO: 59 - DNA, 1128 bp, African swine fever virus
ttaccgcaaattgtctctcagcgaagaaaatgaatgaaacgtttccgtatattcataggttgaaattattttacgcacttcaccaggttctaatatttttttatgaagtattgaatgggaggttaaaagtcctttcttaaaaaagagtttcattataacattcttttcttctctaagaagagtttcttgtattttttttatataaggattggcacccaaacttatacaaaaatgtacattactccaaataccataatttgaaaagaaagttatttccctatctacttcatgattaatgaaacgtatcaacgtctctaaggcagtattgatatttgcacctaaggcaaaacaatagtatatacctaatttattttgagggtacatacaagcaagcgacatcatgtgatttggatctaaacgtatattttcctgaaaatatgcatgatggatttcatcaacattacctaagtatacagccatttttaaacgccaatcatctaggtgaggaaatttcttactaagaaaacgaataggttttataagattaaactctatggcgatcttaaaccaaaattttaatacaagtgtatttcttgtcatttcttctttttcatctaagtataggatataacgattgtaaataaagtttatcaatgcgtaaaaatcatggctatcaaaactgtcgagaatgggaatgttttcataataaatatctatagctaataagaccttttgttgtttaaatagatcaacaaacatagtatacaaccctacctctaaaaatgttggatcagctcctagttgaatacacagaactttcgtcctttccatcttggcacatatgatgccataattgatgttggcaccccataaaacaaataacttgattagatcagtctgatttttctttacagccttcaccaaggctctgtcaagctcatagctgtcaacgtcagagcatgacatagagccactggttaccattttacattgtttacaaaaacctataggtccgttttcccaccataattctagctgctgtaaaataaaaacatcatcttcatgataatttgaaaaagccttgttttctatcaagactttttttgtaagaacctgtaaagaattcat

SEQ ID NO: 60 - DNA, 1122 bp, African swine fever virus
ttaccgcacattgtctctcagcgaagaaaatgaatgaaacgtttccgtatattgataggttgaaattattttacgcacttcactaggttctaatattttcttatgaagtattgaatgggggcttaaaagtcctttcttaaaaagtagtttcattatatcatacttttcttctctaagaagaatttcttgtattttttttgtataaggattggcacccaaacttatacaaaaatgtacattactccaaataccataatttgaaaagaaaggtctatttagttcatgattaatgaaacgtatcaacgcctctaaggcagtattgatatttgcccctaaggcaaaacaatagtatatacctaatttattctgaggatacatacaagcaagcgacatcatgtgatttggatctaaacgtatattttcctgaaaatatgcatgatggatttcatcaacattccctaagtatacagccgtttttaaacgccaatcatctaggtgaggaaatttcttaccaagaaaacgaataggttttacaagattaaactctatggcaaacttataccaaaattttaatacaagtgtattttttgttattttttctttttcatctaaatttaagataaaaccattgtaaataaagtctatcaacacgtaaaaatcatggctatcaaaactgtcgagaatcgaaatattatcataataaatatctatagctaataagaccttttgttgtttaattagatcaacaaacatattatacaaccctacatctaaaaattttggatcagctcctagttgaatacatagaactttcgtcctttccgtcttggcacacacgatgccaaaattaatttcggcgccccataaaacaaataacttgattagatcagtttggttttccttcacagcttttactaaggctctgtcaagctcatagctgtcgacatcagaacatgacatagagccaccggttaccattttacattgtttacaaaaacctatgggtccgttttcccaccacagtccaagctgctgtaaaatgaaactatcatcttcatgataatttgaaaaagccttgttttctatcaagactttttttgtaagaacctgtaaagagttcat

SEQ ID NO: 61 - DNA, 1128 bp, African swine fever virus
ttaccgcaaattatctctcagtgaagaaaatgaatgaaacgtttccgtatgttcataggttgaaattattttacgcacctcactaggttctaatatttttttatgaagtattgaatgggggcttaaaagtccttttttaaaaagtagtttcattataacattcttttcttctctaagaagagtttcttgtattttttttgtatcagggttggcacccaaacttatacaaaaatgtatattactccaaataccataatttgaaaagaaagttatttccctatttacttcatgattaatgaaacgtatcaacgtctctaaggcagtattgatatttgcgcctaaggcaaaacaatagtatatacctaatttattctgagggtacatacaagcaaacgacatcatgtgatttggatctaaacgtatattttcttgaaaatatgcatgatggatttcatcaacattccctaagtatacagccgtttttaaacgccaatcatctaggtgaggaaatttcttactaagataacggataggttttacaagattaaactctatggcgaacttaaaccaaaattttaatacaaatgtgtttcttgtcattttttctttttcatctaaatttaggataaaacgattgtaaataaagtttatcaacgcgtgaaaatcatggctattaaagctgtcgagaatcaaaatattatcataataaatatctatggctaataagaccttttgttgtttaattagattaacaaacatattatacagctttttatctaaaaattctggatcggctcctagttgaatacacaggtctttcgtcctttccatcttagcacacataatgccataattaatgttggcaccccataaaacaaataatatgattagatcagtttggttttccttcacagcttttactaaggccctgtcaagctcatagctgtcaacatcagagcatgacattgagccaatggttactattttacattgtttacaaaaacctatggggccgttttcccaccacaatcctagctgctgtaaaataaaactatcatcttcatgataatttgaaaaggccttgttttctattaagactttttttgtaagaacctgtaaagaattcat

SEQ ID NO: 62 - DNA, 1038 bp, African swine fever virus
ctaggccaacatactttttaatttatagttttttaatagatgatatattttgttaggatctgcttcttttaacgttaatagcgaggagtctgcactataaatgtctaatgataaatgatgagatatcaaatagtaattccgttgctctgctagggcctttgcctcttcaaaggcgtcggctcccagatctatacaaaagaacaagttatccatattataaaatcgtacgcaggcaagcatagctgaattaatattagctcctaagagaaaacaataatatatggttaaaaaattgttatcttttgtgcaggccatccgcatcatttcatccacgtccatgcggatcttttccttttcatacaaattatgtaggtcaaacagcttattaaaacaaagagcacagattaaccaccacgtatttagatacttaaaatgttggtaaacataagaaatggcctccctaagattatcctgcaatgccactataaaacagtatatcgttaacatatcaccatccgacatattacttaatatgtcggtgtcttctactaaccttttcaacttccaatatatggatgaccttatttcccttataatgacataggctggaaagggattatcattaaaaagtttaagacataagataatattactgctagtagtgccagggtgtattaatttaaagaacatgtgcataatcttctttttatccacgcggtacttggctcctaattcccagcaaaattctcgaacaggcggcgtattggcgcaaattaacccatagttgatgtctgcgccccattctgtaaacagttttattaactgatagttgttttcctttgtagccaacattagtgccgtattaaggtccaagccgtctgcaaagcttggcagctttatcagcatatgtttgcaatcaagggaaattggggccttataccaccatagtccgcagcgttctaagataacatggtactcaatagatacttgctgtctggctagtacctttttggcgaaggattgtaaggaaggaaacat

SEQ ID NO: 63 - DNA, 1038 bp, African swine fever virus
ctaggccaacatactttttaatttatagttttttaatagatgatatattttgctaggatctgcttcttttaacgttaatagcgaggagtctgcactataaatgtctaatgataaatgatgagatatcaaatagtaattccgttgctctgctagggcctttgcctcttcaaaggcgtcggctcccagatctatacaaaagaacaagttatccatattataaaatcgtacgcaggcaagcatagctgaattaatattagctcctaagagaaaacaataatatatggttaaaaaattgttatcttttgtgcaggccatccgcatcatttcatccacgtccatgcggatcttttccttttcatacaaattatgtaggtcaaacagcttattaaaacaaagagcacagattaaccaccacgtatttagatacttaaaatgttggtaaacataagaaatggcctccctaagattatcctgcaatgccactataaaacagtatatcgttaacatatcaccatccgacatattacttaatatgtcggtgtcttctactaaccttttcaacttccaatatatggatgaccttatttcccttataatgacataggctggaaagggattatcattaaaaagtttaagacataagataatattactgctagtagtgccagggtgtattaatttaaagaacatgtgcataatcttctttttatccacgcggtacttggctcctaattcccagcaaaattctcgaacaggcggcgtattggcgcaaattaacccatagttgatgtctgcgccccattctgtaaacagttttattaactgatagttgttttcctttgtagccaacattagtgccgtattaaggtccaagccgtctgcaaagcttggcagctttatcagcatatgtttgcaatcaagggaaattggggccttataccaccatagtccgcagcgttctaagataacatggtactcaatagatacttgctgtctggctagtacctttttggcgaaggattgtaaggaaggaaacat

SEQ ID NO: 64 - DNA, 1071 bp, African swine fever virus
ttacgacgttgtatcattgatgtgataatccaaataggccaacatactttttgatttatagttgtttaatagatggtatatttttttagggttcgcttcttttagcgtgaatagagagggatctggatgatagatatctaatgacaaacgttgtgatattaaataatagtcccgctgctctgctagggcttttgcctcttcgaaggcattggctcccaagtctatacaaaagaacagattatctatattaaagaactgtatggcagcaagcatggccatattgatatcggcccctaaaataaaacaataatacatcgttaaaaagttattatctttcgtgcaagctatgcgcatcatttcattaatgtctatgcgaatcttttcctgctcatagagataatggaggtcagacagtttattaaaatacagagcacagattaaccaccacgtattcatgtgcttaaaatgttcgtaaaaatacgagacggcctccctaagatcattctgcaaggccaccataaaacaatatatagttagcatatcgccgtccgaaacatcacgtagtaggtcttcatcttctactaacctcctcagcctccaataaagagacgactttatttcccttataatcacataggtaggaaaaggattctcattaaaaagcttatgacataagataatatattgacttgttttgttctctataaatttaaagaacatgtgtatcaccttttttttatccatgcgatagttcgctcctaattcccaacaaaactctcgggtaggctgtgtattggcacaaatgtatccatagttgatgtttgcaccccactccgtgaatagttttattaattgatagttgttttcctttgcagctatcatcaatgccgtattgagatccaggccatccgcaaagtatggcaaccttattaacatgtgtttgcaatcaagtgaaattggagccgtataccaccataacccgcagtatttcaaaatatgatagtagtcttcaggcagcacatgctgactggctaatatctttttggcgaaagactgtaaagaaggaaacat

SEQ ID NO: 65 - DNA, 1071 bp, African swine fever virus
ttacgacgttgtatcattattgtgataatccaaataggccgacatactttttgatttataattgtttaatagatggtatatttttttagggttcgcttcttttagcgtgaatagagagggatctggatgatagatatctaatgacaaacgatgtgatattaaataatagtcccgctgctctgctagggcttttgcctcttcgaaggcattggcccccaagtctatacaaaagaacagattatctatattaaaaaactgtatggcagcaagcatcgccatattgatatcggcccctaaaataaaacaataatacatcgttaaaaagttgttatctttcgtgcaagctatgcgcatcatttcattaatgtctatgcgaattttttcctgctcatagagataatggaggtcaaacagtttattaaaacacagagcacagattaaccaccacgtattcaggtgcttaaaatgttggtaaaaatacgagacggcctccctaagaccattctgcaaggccaccataaaacaatataaagttagcatatcgccgtccgaaacatcgcgtagtaggtcttcgtcttctactaacctcctcagcctccaataaagagacgactttatttcccttataatcacataggtaggaaaaggattctcattaaaaagcttatgacataagataatatattgactcgttttgttctctataaatttaaaaaacatatgcatcaccttttttttatctaagcgatagttcgctcctaattcccaacaaaactctcgagtaggctgtgtattggcacaaatgtatccatagttgatgtttgcaccccactccgtgaacagttttattaattgatagttgttttcctttgcagctatcatcaatgccgtattgaggtccaggccatccgcaaagtatggcaaccttattaacatgtgtttacaatcaagtgaaattggagccttataccaccataacccgcagcgcttcaaaatatgatagtagtcttcaggcagcacatgctggctggctaatatctttttggcgaaagattgcagggaaggaaacat

SEQ ID NO: 66 - DNA, 1071 bp, African swine fever virus
ttacgacgttgtatcattaatgtgataatccaaataggccgacatactttttgatttatagttgtttaatagatggtatatttttttagggttcgcttcttttagcgtgaatagagagggatctggatgatagatatctaatgacaaacgatgtgatattaaataatagtcccgctgctctgctagggcttttgcctcttcgaaggcattggcccccaagtctatacaaaagaacagattatccatattaaagaactgtatggcagcaagcatggccatattgatatcggcccctaaaataaaacaataatacatcgttaaaaagttgttatctttcgtgcaagctatgcgcatcatttcattaatgtctatgcgaattttttcctgctcatagagataatggaggtcaaacagtttattaaaacacagagcacagattaaccaccacgtattcaggtgcttaaaatgttggtaaaaatacgagacggcctccctaagatcattctgcaaggccaccataaaacaatataaagttagcatatcgtcgtccgaaacatcgcgtagtaggtcttcgtcttctactaatctcctcagcctccaataaagagacgactttatttcccttataatcacataggtaggaaaaggattctcattaaaaagcttatgacataagataatatactgactcgttttgttctctataaatttaaaaaacatatgcatcaccttttttttatctaagcgatagtttgctcctaattcccaacaaaactctcgagtaggctgtgtattggcacaaatgtatccatagttgatgtttgcaccccactccgtgaacagttttattaattgatagttgttttcctttgcagctatcatcaatgccgtattgaggtccaggccatccgcaaagtatggcaaccttattaacatgtgtttacaatcaagtgaaattggagccttataccaccataacccgcagcgcttcaaaatatgatagtagtcttcaggcagcacatgctggctggctaatatctttttggcgaaagattgcagggaaggaaacat

SEQ ID NO: 67 - DNA, 1071 bp, African swine fever virus
ttataacgatgtatcattgatgtcataattcaaataggccaacatactttttgatttatagttttttaatagacgatatattttgttaggatctgcttcttttaacgttaatagcgaggagtctggactataaatgtctaatgataaacgatgagatatcaaatagtaattctgttgctctgccaaggcttttgcctcttcaaaggcatcggcccccagatctatacaaaagaacaggttatccatattatagaatcgtatggaggcaatcatggccaaattaatattagctcctaagataaaacaataatatatagttaaaaaattgttatcttttgtgcaggctatccgcatcatttcatccatgtccatacggattttttctttttcgtacaaattatgtaggtcaaacagcttattatagcaaagagcacatgttaaccaccacgtattcagatacttaaaatgttggtaaacataagaaatggcctccctaagattatcctgcaatgccacgataaaacagtatatagttaacatatcaccatccgaaatattacttaatacgttggtatcttctgctaaattttttagcttccaatgtatacacgactttatttcccttataatgacataggctgaaaagggattgtcattaaaaaatttaagacataagataatattattactagtagtgttaggatgtattaatttaaagaatatgtgcataatcttctttttatccacttggtacttggctcctaattcccagcaaaattctcgaataggtggcgtattcgcgcaaattaacccatagttgatgtctgcgccccaatccgtaaacatttttattaattgatagttgttttcctttgtagccaacattagtgccgtattaaggtccaagccatctgcaaagtttggcagctttatcagcatatgtttgcaatcaagtgaaattggagccttataccaccatagcccgcagcgttctaagataacatggtaatcaatagatacatgctgactggctaatacctttttggcgaaggattgcaaggaaggaaccat

SEQ ID NO: 68 - DNA, 1071 bp, African swine fever virus; identical to SEQ ID NO: 67

SEQ ID NO: 69 - DNA, 1071 bp, African swine fever virus
ttataacgatgtatcattgatgtcataattcaaataggccaacatactttttgatttatagttttttaatagacgatatattttgttaggatctgcttcttttaacgttaatagcgaggagtctggactataaatgtctaatgataaacgatgagatatcaaatagtaattctgttgctctgccaaggcctttgcctcttcaaaggcatcggcccccagatctatacaaaagaacaggttatccatattatagaatcgtatggaggcaatcatggccaaattaatattagctcctaagataaaacaataatatatagttaaaaaattgttatcttttgtgcaggctatccgcatcatttcatccatgtccatacggattttttctttttcgtacaaattatgtaggtcaaacagcttattatagcaaagagcacatgttaaccaccacgtattcaaatacttaaaatgttggtaaacataagaaatggcctccctaagattatcctgcaatgccacgataaaacagtatatagttaacatatcaccatccgaaatattacttaatacgttggtatcttctgctaaattttttagcttccaatgtatacacgactttatttcccttataatgacataggctgaaaagggattgtcattaaaaaatttaagacataagataatattactactagtagtgctaggatgtattaatttaaagaatatgtgcataatcttctttttatccacttggtgcttggctcctaattcccagcaaaattctcgaataggtggcgtattcgcgcaaattaacccatagttgatgtctgcgccccaatccgtaaacatttttattaattgatagttgttttcctttgtagccaacattagtgccgtattaaggtccaagccatctgcaaagtttggcagctttatcagcatatgtttgcaatcaagtgaaattggagccttataccaccatagcccgcagcgttctaagataacatggtgatcaatagatacatgctgactggctaatacctttttggcgaaggattgcaaggaaggaaacat

SEQ ID NO: 70 - DNA, 1071 bp, African swine fever virus
ttataacgttgtattattgacgtcataattcaaataggccaatatactttttgatttatagttttttaatagatgatatattttgttaggatccgcttcttttaacgttaataacgaggaatctggactataaatgtctaatgataaacaatgagatatcaaaaagtaattccgttgctctgccagggcttttgcctcctcaaaggcatcggcccccaggtctatacaaaagaataagttatctatgttatagaattgtatggaggcaatcatagctaaattaatattagctcctaagataaaacaataatatatggttaaaaaattgttatcttttgtgcaggctatccgcatcatttcatccatgtccatgcggatcttttccttttcatacaaatgatgtaggtcaaacaccttattatagcaaagaacacatgttaaccaccacgtattcagatacttaaaatgttggtaaacataagaaatggcctccctaagattatcctgcaatgccacgataaaacagtatatagttaacatatctccatccgaaatattacttaatacgttggtatcttctgctaacttttttagcttccaatgtatacacgactttatttcccttataatgacataggctgaaaagggattgtcattaaaaagtttaagacataagataatattactactagtagtgctaggatgtattaatttaaagaatatgtgcataatcttctttttatccacttggtacttggctcctaattcccagcaaaattctcgaacaggtggcgtattcgcgcaaattaacccatagttgatgtctgcgccccaatccgtaaacatttttattaattgatagttgttttcctttgtagccaacattagtgccgtattaaggtccaagccatctgcaaagtttggcagctttatcagcatatgtttgcaatcaagtgaaattggagccttataccaccatagcccgcagcgttctaagataatatggtgatcaatggatacatgctgtctggctagtacctttttggcgaaggattgtaaggaaggaaacat

SEQ ID NO: 71 - DNA, 1071 bp, African swine fever virus
ttataacaatgtatcattgatatcatcattcaaataggccaacatattttttgatttatagttttttaatagatgatttattttgttaggatctgtttcttttaacgttaatagcaaggagtctggcttataaatgtctaatgataaacgatgagatattaaatagtaattccgttgctctgccagggcttttgcctcttcaaaggcgtcggctcccagatctatacaaaagaacaagttatccatattataaaatcgtacgcaggcaagcatagctgaattaatattagctcctaagagaaaacaataatatatagttaaaaaattgttatcttttgtgcaggccatccgcatcatttcatccacgtccatgcggatcttttccttttcatacaaattatgtaggtcaaacagcttattaaaacaaagagcacagattaaccaccacgtatttagatacttaaaatgttggtaaacataagaaatggcctccctaagattatcctgcaatgccactataaaacagtatatcattaacatatcaccatccgacatattacttaatatgtcggtgtcttctactaaccttttcagcttccaatatatggatatccttatttcccttataatgacataggctggaaagggattatcattaaaaagtttaagacataagataatattactgctagtagtgccaggatgtattaatttaaagaacatgtgtataatcttctttttatccacgcggtgcttggctcctaattcccagcaaaattctcgaacagacggcgtattggcgcaaattaacccatagttgatgtctgcgccccattccgtaaacagttttattaactgatagttattttcctttgtagccaacattagtgccgtattaaggtccaagccgtctgcaaagcttggcagctttatcagcatatgtttgcagtcaagtgaaattggagccttataccaccatagtccgcaacgttctaagataacatggtactcaatagatacttgctgtctggctagtacctttttggcgaaggattgtaaggaaggaaacat

SEQ ID NO: 72 - DNA, 1062 bp, African swine fever virus
tcatattgtatcatcatggttaaaatatgccgtcatattttttgatttatagttttttaatagatgatatattttgttagggtccgcttcttttaacgttaatagcgaggagtctggactataaatgtctaatgataaacgatgagatatcaaaaagtaattccgttgctctgccagggcttttgcctcttcaaaggcatcggcccccaagtctatacaaaagaacaggttatccatattataaaatcgtatggaggcaatcatggccaaattaatattagctcctaagataaaacaataatatatagttaaaaaattgttatcttttgtgcaggctatccgcatcatttcatccatgtccatgcggattttttccttttcgtacaaattatgtaggtcaaacagcttattaaaacaaagagcacagattagccaccacgtattcagatatttaaaatgttggtaaacataagaaatggcctccctaagattatcctgcaatgccaccataaaacagtatatcgttaacatatcaccatccgaaatattacttaatacgtcggtgtcttctactaactttttcagcttccaatatatggatgactttatttcccttataatgacataggctgaaaaggggttatcattaaaaagtttaagacataagataatattactgctagtagtgccaggatgtattaatttaaagaacatgtgcataatcttctttttatccacgcggtacttggctcctagttcccagcacaattctcgaacaggcggcgtattggcgcaaattaacccatagttgatgtctgcgccccattccgtaaacagttttattaactgatagttgttttcctttgtagccaacattagtgccgtattaaggtccaagccatctgcaaagcttggcagctttatcagcatatgtttgcaatcaagggaaattggagccttataccaccatagtccgcagcgttctaagataacatggtaatcaatagatacatgctgtctggctagtacctttttggcgaaggattgtaaggaaggaaacat

SEQ ID NO: 73 - DNA, 1062 bp, African swine fever virus
tcatattgtatcatcatggttaaaatatgccgtcatattttttgatttatagttttttaatagatgatatattttgttagggtccgcttcttttaacgttaatagcgaggagtctggactataaatgtctaatgataaacgatgagatatcaaaaagtaattccgttgctctgccagagcctttgcctcttcaaaggcgtcggcccccaggtctatacaaaagaacaggttatccatattatagaatcgtatggaggcaatcatggccaaattaatattagctcctaagataaaacaataatatatagttaaaaaattgttatcttttgtgcaggctatccgcatcatttcatccatgtccatgcggattttttccttttcgtacaaattatgtaggtcaaacagcttattaaaacaaagagcacagattagccaccacatattcagatacttaaaatgttggtaaacataagaaatggcctccctaagattatcctgcaatgccaccataaaacagtatatcgttaacatatcaccatctgaaatatcacttaatacgtcggtgtcttctactaactttctcagcttccaatatatggatgactttatttcccttataatgacataggctgaaaaggggttatcattaaaaagtttaagacataagataatattactgctagtagtgccaggatgtattaatttaaagaacatgtgcataatcttctttttatccacgcggtacttggctcctagttcccagcacaattctcgaacaggcggtgtattggcgcaaattaacccatagttgatgtctgcgccccattccgtaaacagttttattaactgatagttgttttcctttgtagccaacattagtgccgtattaaggtccaagccatctgcaaagcttggcagctttatcagcatatgtttgcaatcaagtgaaattggagccttataccaccatagtccgcagcgttctaagataacatggtaatcaatagatacatgctgtctggctagtacctttttggcgaaggattgtaaggaaggaaacat

SEQ ID NO: 74 - DNA, 1068 bp, African swine fever virus
ttacactgtgtcatgcccatcatagtcaaaatataccatcatattttttgatttatagttttttaatagatgatatatttttttaggatctgcttcttttaacgttaatagcgaggagtctggactataaatatctattgataaacgatgatacatcaaaaagtaattccgctggtctgccagggcttttgcctcttcaaaggcatcggctcccaggtctatacaaaagaacaagttatccatattatagaattgtacgcaggcaagcatagcctgattaatattagctcctaagagaaaacagtaatatatggttaaaaagttgttatctttagtgcaggctatgcacatcatttcatccatgtccatgcggatcttttccttttcatacaaatcatgtaggtcaaacagcttattaaaacaaagagcacagattaaccaccacgtattcagatgcttaaaatgttggtaaaaataagaaatggcctccctaagattatcctgcaatgccaggataaaacagtatatagtcaacatatcatcatccgacatattacttaatatgtcagtgtcttctactaaccttctcagcttccaatatatagacgactttatttcccttataatgacataggttggaaaaggattattattaaaaagtttaagacataagataatattactactagtagtgccatgatgtattaatttaaaaaacatatgcataaccttatttttatccacttggtacttggctcctaattcccagcaaaattctcgaacaggcggcgtattagcgcaaattaatccatagttgatgtccgcgccccattccgtaaacagttttattaactgatagttgttttcctttgtagccaacattagtgccgtattaaggtccaagccatctgcaaagcttggcagctttattaacatatgtttgcaatcaagtgaaattggagccttataccaccatagtccgcaatgttttaagatataatgataatcaatggatacatgatgtctagctaatacctttttggcgaaggattgtaaagaaggaaacat

SEQ ID NO: 75 - DNA, 1062 bp, African swine fever virus
ttatctttgttcataatcaagaaaaatccccatatttattgagtgataattttttaacatgcaatttattttttcagggtccgtaacgatcgacaacagagaaataaccggattgtaatgctttaatgataaggcatgggctatcagataattttccttttgttctgccaaagctttgccctcctcaaaggcatcggcacccaggtctatacaaaagaacaggtttccaagattatagttttgtatggaaacaagcatggcttgattgatgttggctcccatgataaaacagtagtaaatggccgaatagctataatcttggatgcaggctatgtgcatcatttcatcaatatccatgcggaccctttctatttcgtacagctcgtgaaggtcgaacacgttgttgtaaaaaagggcgcacatgagccgccacctatgtagacgcgggtatttctggtaaaagtagcggatagcatctttgaggtcatagtccaccgctatcgcgtaccagtatttggttaaaacagtgctaaagctatcatcatggtccagcatgaaggttatctccatgagccctcttaactcccacatgatttcccccctcagatccagattatctataatccttaaattggggttattggaaaacacctcgtggcaaaagataatattgctactggttttatcgcgcgttgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaaacaggttgaccttataaataatttattgcgtcgaatatgaagcataatggggccattatgccaccacaggccacaacacttcaggacatgatattgatctaccggtatacactgcccggccagtactttcttcgtgagggattgcagggaaggcaacat

SEQ ID NO: 76 - DNA, 1062 bp, African swine fever virus; identical to SEQ ID NO: 75

SEQ ID NO: 77 - DNA, 1062 bp, African swine fever virus
ttatctttgttcataatcaagaaaaatccccatatttattgagtgataattttttaacatgtaatttattttttcagggtccataacgttcgacaacagagaaatgactggattgtaatgctttaatgataaggcatgggctatcagataattttccttttgttctgccaaagctttgccctcctcaaaggcgttggcgcccaggtctatacaaaagaacaggtttccaagattatagttttgtatggaaaccagcatggcttgattgatgttggctcccatgataaaacagtagtaaatggccgaatagctataatcttggatgcaggctatgtgcatcatttcatcgatatccatgcggaccctttctatttcatacagctcgtgaaggtcgaacacgttgttgtaaaaaagggcgcacatgagccgccaccgatgtaggcgcgggtatttctggtaaaagtagcgaatagcatctttgaggtcatagtccaccgctatagcataccagtatttggttaaaacagtgctaaagctatcatcatggtctagcatgaaggttatctccatgagccctcttaactcccacatgatttcccccctcagatccagattatctataatccttaaattggggttattggaaaacacctcgtggcaaaagataatattgctactggttttatcgcgcgtcgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatagtttattacgtcgaatatgaagcataatggggccattatgccaccacaggccacaacacttcaggatatgataatgatccaccgacacgcactgcccggccagtactttcttcgtgagggattgcagggaaggcaacat

SEQ ID NO: 78 - DNA, 1062 bp, African swine fever virus; identical to SEQ ID NO: 77

SEQ ID NO: 79 - DNA, 1062 bp, African swine fever virus
ttatctttgttcataatcaagaaaaatccccatatttattgagtgataattttttaacatgtaatttattttttcagggtccataacgatcgacaacagagaaatgaccggattgtaatgctttaatgataaggcatgggctatcagataatttcccttttgttctgccaaagctttgccctcctcaaaggcgttggcgcccaggtctatacaaaagaacaggtttccaagattatagttttgtatggaaaccagcatagcttgattgatgttggctcccatgataaaacagtagtaaatgaccgaatagctataatcttggatgcaggctatgtgcatcatttcatcaatatccatgcggaccctttctatttcatacagctcgtgaaggtcgaacacgttgttgtaaaaaagggcgcacatgagccgccacctatgtaggcgcgggtatttctggtaaaagtagcgaatagcatctttgaggtcatagtccaccgctatagcataccagtatttggttaaaacagtgctaaagctatcatcatggtctagcatgaaggttatctccatgagccctcttaactcccacatgatttctcccctcagatccagattatctataatccttaaattggggttattggaaaacacctcgtggcaaaagataatattgctactggttttatcgcgcgtcgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatagtttattacgtcgaatatgaagcataatggggccattatgccaccacaggccacaacacttcaggatatgataatgatccaccgacacgcactgcccggccagtactttcttcgtgagggattgcagggaaggcaacat

SEQ ID NO: 80 - DNA, 1062 bp, African swine fever virus
ttatctttgttcataatcaagaaaaatccccatatttattgagtgataattttttaacatgcgatttattttttcagggtccgtaaccaccgacaacagagaaatgaccggattgtaatgctttaatgataaggcattggctatcagataatttcccttttgttctgccaaagctttgccctcctcaaaggcgttggcacccaggtctatacaaaagaacaggttcccaagattatagttttgtatggaaaccagcatggcttgattgatgttggctcccatgataaaacagtagtagatggctgaatagctataatcttggacgcaggctatgtgcatcatttcatcaatgtccatgcggaccctttctatttcatacagctcgtgaaggtcgaacacgttgttgtaaaaaagggcgcacatgagccgccacctatgtagccgcgggtatttctggtaaaagtagcggatagcatctttgaggtcatagtccaccgctatcgcgtaccagtatttggttaaaacagtgctaaagctatcatcatggtccagcatgaaggttatctccatgagccctcttaactcccacatgatttcccccctcagatccagattatctataatccttaaattggggttattggaaaacacctcgtggcaaaagataatattgttactggttttatcgcgcgttgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatattctattacgtcgaatatgaagcattatggggccattatgccaccacaggccacaacacttcaggatatgataatgatccaccgacacgcactgcccggccagtactttcttcgtgagggattgcagggaaggcaacat

SEQ ID NO: 81 - DNA, 1062 bp, African swine fever virus
ttatctttgttcataatcaagaaaaatccccatatttattgagtgataattttttaacatgcgatttattttttcagggtccgtaaccaccgacaacagagaaatgaccggattgtaatgctttaatgataaggcattggctatcagataatttcccttttgttctgccaaagctttgccctcttcaaaggcgttggcacccaggtctatacaaaagaacaggttcccaagattatagttttgtatggaaaccagcatggcttgattgatgttggctcccatgataaaacagtagtaaatggctgaatagctataatcttggacgcaggctatgtgcatcatttcatcaatgtccatgcggaccctttctatttcatacagctcgtgaaggtcgaacacgttgttgtaaaaaagggcgcacatgagccgccacctatgtagccgcgggtatttctggtaaaagtagcggatagcatctttgaggtcatagtccaccgctatcgcgtaccagtatttggttaaaacagtgctaaagctatcatcatggtccagcatgaaggttatctccatgagccctcttaactcccacatgatttcccccctcagatccagattatctataatccttaaattggggttattggaaaacacctcgtggcaaaagataatattgttactggttttatcgcgcgttgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatattctattacgtcgaatatgaagcattatggggccattatgccaccacaggccacaacacttcaggatatgataatgatctaccgacacgcactgcccagccagtactttcttcgtgagggattgcagggaaggcaacat

SEQ ID NO: 82 - DNA, 1062 bp, African swine fever virus
ttatctttgttcataatcaagaaaaatccccatatttattgagtgataattttttaacatgcaatttatttttttagggtctgtaacgaccgacaacagagaaatgaccggattgtaatgctttaatgataaggcatcggctatcagataatttcccttttgttctgccaaagctttgccttcctcaaaggcgttggctcccaggtctatacaaaagaacaggttcccaagattatagttttgtatggaaaccagcatggcttgattgatgttggctcccatgataaaacagtagtaaatggccgaatagctataatcttggacgcaggctatgtgcatcatttcatcaatgtccatgcggaccctttctatttcgtacagctcgtgaaggtcgaacacgttgttgtaaaaaagggcgcacatgagccgccacctatgtagacgcgggtatttctggtaaaagtagcggatcgcatctttgaggtcatagtccaccgctatcgcgtaccagtatttggttaaaacagtgctaaagctatcatcatggtccagcatgaaggttatctccatgagccctcttaactcccacatgatttcccccctcagatccagattatctataatccttaaattggggttattggaaaacacctcgtggcaaaagataatattgttactggttttatcgcgcgtcgtatcaaagaaaatttttaaaatatactctctttctaaatattctttggcacccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatagtttattacgtcgaatctgaagcataatgggtccattatgccaccacaggccacaacacttcaggatatgataatgatccaccgacacgcactgcccggccagtactttcttcgtgagggattgcagggaaggcaacat

SEQ ID NO: 83 - DNA, 1062 bp, African swine fever virus
ttatctttgttcataatcaagaaaaatccccatatttattgagtgataattttttaacatgcgatttattttttcagggtccgtaaccaccgacaacagagaaatgaccggattgtaatgctttaatgataaggcattggctatcagataatttcccttttgttctgccaaagctttgccctcctcaaaggcgttggcccccaggtctatacaaaagaacaggttcccaagattatagttttgtatggaaaccagcatggcttgattgatgttggctcccatgataaaacagtagtaaatggctgaatagctataatcttggacgcaggctatgtgcatcatttcatcaatgtccatgcggaccctttctatttcatacagctcgtgaaggtcgaacacgttgttgtaaaaaagagcgcacatgagccgccacctatgtagccgcgggtatttctggtaaaagtagcggatagcatctttgaggtcatagtccaccgctatcgcgtaccagtatttggttaaaacagtactaaagctatcatcatggtccagcatgaaggttatctccatgagccctcttaactcccacatgatttctccccttagatccagattatctataatccttaaattggggttattggaaaacacctcgtggcaaaagataatattgttactggttttatcgcgcgttgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatattctattacgtcgaatatgaagcattatggggccattatgccaccacaggccacaacacttcaggatatgataatgatccaccgatacgcactgcccggccagtactttcttcgtgagggattgcagggaaggcaacat

SEQ ID NO: 84 - DNA, 1062 bp, African swine fever virus
ttatcttcgttcataatcaagaaaaaccctcatatttattgagtgataattttttaacatgcaatttattttttcagggtccataatgaccgacaacagagaaatgaccggattgtaatgctttaatgataaggcatcggctatcagataattttccttttgttctgccaaagctttcccctcctcaaaggcgttggcccccagatctatacaaaagaacatgttcccaagattatagttttgtatagaaacaagcatggcttgattgatgttggcccccatgataaaacagtaataaatagccgcatagctataatcttggacgcaggctatgtgcatcatttcatcaatatccatgcggaccctttctattacatacagctcgtgaaggtcgaacacgttgttgtaaaaaagggcgcacataagccgccagtggtgtagatgggtatacctctgataaaaataatggatagcctttttgaggccatagtctaccgctatggcgtaccagtatttggttaacatattgctaaaggagttgtcatggtccaacatgtaggttatctccatgagtccccttagctcccacatgatttcctccctcagatccaaattatccacatttttcaggttggggttattggaaaacatttcgtggcataagataatattactgtcggttttgttgcgcgtcgtatcaaagaaaatttttaaaatataatctctatctaaacgatctttggctcctagttctctgcatagattacgggtgtgctccgtatgggcacaaatcagtccataattaatatcggcaccccactcggcaaacagccttatcaagtcatggttgttttccttcgcggctttcatcagtacggtgttcaattcaataccctcgctaaaacaggttgaccttataaacattttgtgacgataaacgtaaaatgtaatagggccatcataccaccacaacccgcagcactttaagacatgatattgatctaccggtacacactgtttggccagcactttcttagttaaagattgtaaggaaggcaacat

SEQ ID NO: 85 - DNA, 1062 bp, African swine fever virus
ttatctgcgttcataatcgagaaaaaccaccatattctttgagtgataattttttaacatagaatttatcttttctgggtcggtcacggccgacaacagggaaatcaccgggttataatgttttaatgataaggcatcggctatcaaataattttccttttgtattgctagggttttgccttcttcaaaggcgttggcacccaggtctatacaaaagaacatgttcccgagattatagttttgtatggaaacaaacatggcttgattgatgttggcccccatgataaaacagtagtagatagctgaatagctataatcttggatgcaggctatgtgcatcatctcatcgatatccatgcggaccctttctatttcatacagctcgtgaaggtcgaacacattgttataaaaaagggcgcacataagccgccaccgatgtagatgggtatacttctgataaaaatagtggatggcctttttgaggccgtagtctaccgctatggcgtaccagtatttggttaacatattgctgaaggagttgtcatggtccaacatgtaggttatctccatgagtccccttagctcccacatgatttcctccctcagatccaaatcatccacatgtttcagattggggttattggaaaacatttcgtggcataagataatattactgtcggttttgttgcgcgtcgtatcaaagaaaatttttaaaatatactctctatctaaacggtctttggctccaagttctctgcatagattgcgggtgtgctccgaatgagcacaaaccagtccataattaatatcggcaccccactcaacaaacagccttatcaagtcatgattgttttccttcgcggctttcatcagtacggtgttcaattcaataccctcgctaaaacaggttgactttataaacattttgttacgatacgtataaaatgtaatagggccatcataccaccacaacccgcagcatttcaggatatgatattgatctaccggtatacactgtttggccagcactttcttggatagggattgcaaggaaggcaacat

SEQ ID NO: 86 - DNA, 1062 bp, African swine fever virus
ttatctgcgttcataatcgagaaaaaccaccatattctttgagtgataattttttaacatagaatttatcttttctgggtcggtcacggccgacaacagggaaatcaccgggttataatgttttaatgataaggcatcggctatcaaataattttccttttgtattgctagggctttgccttcttcaaaggcgttggcacccaggtctatacaaaagaacatgttcccgagattatagttttgtatggaaacaaacatggcttgattgatgttggcccccatgataaaacagtagtaaatagctgaatagctataatcttggatgcaggctatgtgcatcatctcatcgatatccatgcggaccctttctatttcatacagctcgtgaaggtcgaacacattgttataaaaaagggcgcacataagccgccaccgatgtagatgggtatacttctgataaaaatagtggatggcctttttgaggccgtagtctaccgctatggcgtaccagtatttggttaacatattgctgaaggagttgtcatggtccaacatgtaggttatttccatgagtccccttagctcccacatgatttcctccctcagatccaaatcatccacatgtttcagattggcgttattggaaaacatttcgtggcataagataatattactgtcggttttgttgcgcgtcgtatcaaagaaaatttttaaaatatactctctatctaaacggtctttggctccaagttctctacatagattgcgggtgtgctccgaatgagcacaaaccagtccataattaatatcggcaccccactcaacaaacagccttatcaagtcatgattgttttccttcgcggctttcatcagtacggtgttcaattcaataccctcgctaaaacaggttgactttataaacattttgttacgatacgtataaaatgtaatagggccatcataccaccacaacccgcagcatttcaggatatgatattgatctaccggtatacactgtttggccagcactttcttggatagggattgcaaggaaggcaacat

SEQ ID NO: 87 - DNA, 1062 bp, African swine fever virus
ttatctgcgttcataatcgagaaaaaccaccatattctttgagtgataattttttaacatagaatttatcttttccgggtcggtcacggccgacaacagggaaatcaccgggttataatgttttaatgataaggcatcggctatcaaataattttccttttgtattgctagggctttgccttcttcaaaggcgttggcacccaggtctatacaaaagaacatgttcccgagattatagttttgtatggaaacaaacatggcttgattgatgttggcacccatgataaaacagtagtaaatagttgaatagctataatcttggatgcaggctatgtgcatcatctcatcgatatccatgcggatcctttctatttcatacagctcgtgaaggtcgaacacattgttataaaaaagggcgcacatgagccgccaccgatgtagatgggtatacttctgataaaaatagtggatggcctttttgaggccgtagtctaccgctatggcgtaccagtatttggttaacatgttgctgaaggagttgtcatggtccaacatgtaggttatctccatgagtccccttagctcccacatgatttcctccctcaggtccaaatcatccacatgtttcagattggggttattggaaaacatttcgtggcataagataatattactgtcggttttgttgcgcgtcgtatcaaagaaaatttttaaaatatactctttatctaaacggtctttggcccctagttctctgcatagatcgcgggtgtgctccgaatgagcacaaaccagtccataattaatatcggcaccccactcagtaaataattttattaggtcatgattgttttccttcgcggctttcatcagtacggtgttcaattcaataccctcgctaaaacaggttgactttataaacattttgttacgatacgtataaaatgtaatagggccatcataccaccacaacccgcaacttttcaggatatgatattgatctaccggtatacactgtttggccagcacttttttggatagggattgcaaggaaggcaacat

SEQ ID NO: 88 - DNA, 1053 bp, African swine fever virus
tcatcttaaatcataggtaaggaagatcatcatattttttgaaacgtaattttttaacgcatgatctatgatttcagggtccgtgcttttaggcaacggggtggtggccggactataaatctttagggataaaatgttctttataagctcatacccttcccctaaagctgtagtaccctcttcgaaaacatcagcccccagatctatacaaaagaacatgttttctatattatagtactgtattgagctaagcatggcttgattgatgttggcgcccaggacatagcagtagtacatggttgaaaggttgtggtctttgatgcaggcgatccgcatcatctcttctatgtccatatggatcttgtccttttcatacgcctcatgaaggtcaaacacattattaaaacaaagagcacatgttaaccgccacgtattcaggtgtgtatatttttggtaaaaatactgtatggcctctttcaggttatagcgtatggctatagcgtaccagtatttgagtagtaatgtactgagcgaaaactcattatttagcagatcggtttttactattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtttccttggcacctagctctcgacagaggtcccaggtgtgctccgtgttgacagataccagcccgtagttgatgtccgccccccactctgcaaacagttttataaggttgtagttgttttcccttacagccttcactaacgccgtatttaggtttaagccctctttaatacctgctgattttatgagccttaggttatgatcaaacgtgatcggagcatcatgccaccataggtcataacactttaaaagataatgttggttcgtgggcacgcattgtccagccaacacctttttggtcagagattgcagggaaggcaacat

SEQ ID NO: 89 - DNA, 1053 bp, African swine fever virus; identical to SEQ ID NO: 88

SEQ ID NO: 90 - DNA, 1053 bp, African swine fever virus
tcatcttaaatcataggtaaggaagatcatcatattttttgaaacgtaattttttaacgcatgatctatgatttcagggtccatgcttttaggcaacggggtggtggccggactataaatctttagggataaaatgttctttataagctcatacccttcccctaaagctgtagtaccctcttcgaaaacatcagcccccagatctatacaaaagaacatgttttctatattatagtactgtattgagctaagcatggcctgattgatgttggcgcccaggacatagcagtagtacatggttgaaaggttgtggtctttgatgcaggcgatccgcatcatctcttctatgtccatatggatcttgtccttttcatacgcctcatgaaggtcaaacacattattaaaacaaagagcacatgttaaccgccacgtattcaggtgtgtatatttttggtaaaaatactgtatggcctctttcaggttatagcgtatggctatagcgtaccagtatttgagtagtaatgtactgagcgaaaactcattatttagcagatcggttttttctattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtctccttggcacctagctctcgacagaggtcccaggtgtgctccgtgttgacagataccagcccgtagttgatgtccgccccccactctgcaaacagttttataaggttgtagttgttttcccttacagcctttactaacgccgtatttaggtttaagccctctttaatacctgctgattttatgagccttaggttatgatcaaacgtgatcggagcatcatgccaccataggtcataacactttaaaagataatgttggttcgtgggcacgcattgtccagccaacacctttttggtcagagattgcagggaaggcaacat

SEQ ID NO: 91 - DNA, 1053 bp, African swine fever virus
tcatcttaaatcgtaggtaaggaagatcatcatattttttgaaacgtaattttttaacgcatgatctataatttcagggtccgtgcttttaggcaacggggtggtggccggactataaatctttagggataaaatgttctttataagcttatacccttcccctaaagctatagtgccctcttcgaaagcatcagcccccagatccatacaaaagaacatgttttctatattatagtactgtattgaggtaagcatggcctgattgatgttggcgcccaggacatagcagtagtacatggttgaaaggttgtggtctttgatgcaggcgatccgcatcatctcttctatgtccatatggatcttgtccttttcatacgcctcatgaaggtcaaacacattattaaaacaaagagcacatgttaaccgccacgtattcaggtgtgtatatttttggtaaaaatactgtatggcctctttcaggttatagcgtatggctatagcgtaccagtatttgagtagtaatgcactgagcgaaaactcattatttagcagatcggttttttctattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtctccttggcacctagctctcgacagaggtcccgggtgtgctccgtgttgacagataccagcccatagttgatgttcgccccccactctgcaaacagttttataaggttgtaattgttttcccttacagccttcactaacgccgtatttaggtctaagccctctttaatgcctgctgattttatgagctttaggttatgatcaaacgtgatcggaccatcatgccaccataggtcataacactttaaaagataatgttggttcgtgggcacgcattgtccagccaacacctttttggtcagagattgcagggaaggcaacat

SEQ ID NO: 92 - DNA, 1053 bp, African swine fever virus
tcatcttaaatcgtaggtaaggaagatcatcatattttttgaaacgtaattttttaccgcatgatctatgatttcagggtccgtgcttttaggcaacggggtggtggccggactataaatctttagggataaaatgttctttataagcttatacccttcccctaaagctatagtaccctcttcgaaagcatcagcccccagatccatacaaaagaacatgttttctacattatagtactgtattgaggtaagcatggcctgattgatgttggcgcccagaacatagcagtagtacatggttgaaaggttgtggtctttgatgcaggcgatccgcatcatctcttctatgtccatatagatcttgtccttttcatacgcctcatgaaggtcaaacacattattaaaacaaagagcacatgttaaccgccatgtattcaggtgtgtatatttttggtaaaaatactgtatggcctctttcaggttatagcgtatggctatagcgtaccagtatttgagtagtaatgtactgagcgaaaactcattatttagcagatcggttttttctattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtctccttggcacctagctctcgacagaggtcccgggtgtgctccgtgttgacagataccagcccatagttgatgtccgccccccactctgcaaacagttttataaggttgtagttgttttcccttacagccttcactaacgccgtatttaggtctaagccctctttaatgcctgctgattttatgagctttaggttatgatcaaacgtgatcggaccatcatgccaccataggtcataacactttaaaagataatgttggttcgtgggcacgcattgtccagccaacacctttttggtcagagattgcagggaaggcaacat

SEQ ID NO: 93 - DNA, 1053 bp, African swine fever virus
tcatcttaaatcgtaggtaaggaagatcatcatattttttgaaacgtaattttttaacgcatgatctataatttcagggtccgtgcttttaggcaacggggtggtggccggactataaatctttagggataaaatgttctttataagcttatacccttcccctaaagctatagtgccctcttcgaaagcatcagcccccagatccatacaaaagaacatgttttctatattatagtactgtattgaggtaagcatggcctgattgatgttgccccccaggacatagcagtagtacatggttgaaaggttgtggtctttgatgcaggcgatccgcatcatctcttctatgtccatatgaatcttgtccttttcatacgcctcatgaaggtcaaacacattattaaaacaaagagcacatgttaaccgccacgtattcaggtgtgtatatttttggtaaaaatactgtatggcctctttcaggttatagcgtatggctatagcgtaccagtatttaagtagtaatgtactgagcgaaaactcattatttagcagatcggttttttctattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtctccttggcacctagctctcgacagaggtcccaggtgtgctccgtgttgacagataccagcccatagttgatgttcgccccccactctgcaaacagttttataaggttgtagttgttttcccttacagccttcactaacgccgtatttaggtctaagccctctttaatgcctgctgattttatgagctttaggttatgatcaaacgtgatcggaccatcatgccaccataggtcgtaacactttaaaagataatgttggtttgtgggcacgcattgtccagccaacacctttttggtcagagattgcagggaaggcaacat

SEQ ID NO: 94 - DNA, 1053 bp, African swine fever virus
tcatcttaaatcataggaaaggaagatcatcatattttttgaaaagtaattttttaacgcatgatctatgatttcagggtccgtgcttttaggcaacgggatggtggtcggactataaatctttagagataaaatgttttttataagctcatacccttcccctaaagctgtagtaccctcttcgaaaacatcagcccccagatccatacaaaagaacatgttttctatattatagtactgtattgaggtaagcatggcctgattgatgttggcgcccaacatatagcagtagtacatggttgaaaggttgtggtctttaatgcaggcgatccgcatcatctcttctatgtccatatagatcttgtccttttcatacgcctcatggaggtcaaacacattattaaaacaaagagcacatgttaaccgccacgtattcaggtgtgtatatttttggtaaaaatactgtatggcttctttcaggttatagcgtatggctatagcgtaccagtatttgagtagcaatgtactgagcgaaaactcattatttagcagatcggttttttctattaactcccttaactcccaaaaaatttctatcctcatttttatattatttactttttgtaatatcggattgttggaaaacacctcatggcataaaataatgttactactagttttatgaaactttagatctataaaaatttgtaaaatttctttttcattcaaggtctccttggcacctagctctcgacagaggtcccaggtgtgctccgagctgacagataccagcccatagttgatgtccgccccccactctgcaaacaattttataaggttgtagttgttttcctttacagccttcaccaacgccgtatttaggtctaagccctctttaatgcctgctgattttatgagccttaggttatgatcaaacgtgatcggagcatcatgccaccataggtcataatattttaaaagataatgttggttcgtgggcacgcattgtccggccaacacctttttggtcaaagattgcagggaaggcaacat

SEQ ID NO: 95 - DNA, 1053 bp, African swine fever virus
tcatcttaaatcataggaaaggaagatcatcatattttttgaaaagtaattttttaacgcatgatctatgatttcagggtccgtgcttttaggcaacgggatggtggtcggactataaatctttagagataaaatgttttttataagctcatacccttcccctaaagctgtagtaccctcttcgaaaacatcagcccccagatccatacaaaagaacatgttttctatattatagtactgtattgaggtaagcatggcctgattgatgttggcgcccaacatatagcagtagtacatggttgaaaggttgtggtctttgatgcaggcaatccgcatcatctcttctatgtccatatggatcttgtccttttcatacgcctcatggaggtcaaacacattattaaaacaaagagcacatgttaaccgccacgtattcaggtgtgtatatttttggtaaaaatactgtatggcctctttcaggctatagcgtacggctatagcgtaccagtatttgagtagtaatgtactgagcaaaaactcattatttagcagatcggttttttctattaactcccttaactcccagaaaatttctatcctcaatttcagattatttactttttgtaatatcggattgttggaaaacacctcatggcataaaataatgttactactagttttatgaaactttagatctatgaaaatttgtaaaatttcctcttcattcaagatctccttggcacctaactcttgacagaggtcccgggtatgctccgtgttgacagataccagcccatagttgatgtccgccccccactctgtaaatagttttataaggctgtagttgttttcccttacagcctttactaacgccgtatttaggtctaagccctctttaatgcctgatgattttatgagccttaggttatgatcaaacgtgatcggagcattataccaccatagatcataatattttaaaagataatgttggttcgtgggcacgcattgtccagccaacacctttttggtcaaagattgcagggaaggcaacat

SEQ ID NO: 96 - DNA, 1053 bp, African swine fever virus
tcatcttaaatcataggaaaggaagatcatcatattttttgaaaagtaattttttaacgcatgatctatgatttcagggtccgtgcttttaggcaacgggatggtggtcggactataaatctttagagataaaatgttttttataagctcatacccttcccctaaagctgtagtaccctcttcgaaaacatcagcccccagatccatacaaaagaacatgttttctatattatagtactgtattgaggtaagcatggcctgattgatgttggcgcccaacatatagcagtagtacatggttgaaaggttgtggtctttgatgcaggcaatccgcatcatctcttctatgtccatatggatcttgtccttttcatacgcctcatggaggtcaaacacattattaaaacaaagagcacatgttaaccgccacgtattcatgtgtgtatatttttggtaaaaatactgtatggcctctttcaggctatagcgtacggctatagcgtaccagtatttgagtagtaatgtactgagcaaaaactcattatttagcagatcggttttttctattaactcccttaactcccagaaaatttctatcctcaatttcagattatttactttttgtaatatcggattgttggaaaacacctcatggcataaaataatgttactactagttttatgaaactttagatctatgaaaatttgtaaaatttcctcttcattcaagatctccttggcacctaactcttgacagaggtcccgggtatgctccgtgttgacagataccagcccatagttgatgtccgccccccactctgtaaatagttttataaggctgtagttgttttcccttacagcctttactaacgccgtatttaggtctaagccctctttaatgcctgatgattttatgagccttaggttatgatcaaacgtgatcggagcattataccaccatagatcataatattttaaaagataatgttggttcgtgggcacgcattgtccagccaacacctttttggtcaaagattgcagggaaggcaacat

SEQ ID NO: 97 - DNA, 1050 bp, African swine fever virus
tcatcttaaatcgtaggaaaggaaggtcatcatattttttgatttataattttttaacacatgatctattatttcagggtccgtgtttttaggtaacggggcagtggacggactatagatctttagggataaaatgttccttataagctcatacccttcccctacaggcatattctcttcgaaaacatcagcccccagatccatacaaaagaacatgttttctatattatagtactgtattgaggcaagcatggcctgattaatgttggcgcccaggacatagcagtagtatatcgttgaaaggttgtggtctttgatgcaggctactcgcatcatctcttctaagtccatatagatcttgtccttttcatacgcctcatgaaggtcaaacacattattaaaacaaagagcacatgttaaccgccacgtattcaggtgtgtatatttttgataaaaatactgtatggcctctttcaggttatagcgtacggctatagcgtaccagtatttgagtaataatgtattgagtgaaaactcattatttaacagatcggttttttttattaactcccttaactcccagaaaatttctatcctcattttcagattatttactttttgtaataacggattgttggaaaacacctcatggcataaaataatgttactactagttttataaaactttagatcgatgaaaattcgtaaaatttcctcttcatttaaggtctccttggcgcctagctctctgcagaggtcccgggtgtgctccgtgttgacagataccagcccgtagttgatgtccgccccccactctgtaaaaagctttataaggttgtagttgttttcccttacagcctttactaacgccgtatttaggtctaagccatctttaatgcctgctgattttatcagccctaggttatgatcaaacgtgatcggagaatcataccaccataggtcgtaacactttaaaagataatattggtccgtcggcaggcattgtccagccagtacctttttggtcagagattgcagggaaggcaacat

SEQ ID NO: 98 - DNA, 1056 bp, African swine fever virus
tcatcttagatcataagtaaggaagaccatcatattttttgaaaagtaattttttaactcatgatctataatttcagggtccgtgcttttaggcaacggggtggtggccgggctataaatctttagggataaaatattccttataggctcatacccttcctgtcccactagagccataccctcttcaaaagcatcggcccccagatccatacaaaagaatatgttttctatattatagtactgtattgaggtaagcatggcctgattgatgttggcgcccagcatatagcagtagtatatcgttgaaaggctgtggtctttgatgcaggctatccgcatcatctcttctaaatccatacagatcttgtccttttcatacgcctcatgaaggtcaaacacgttattaaaacaaagagcacatgttaatcgccacgtattcaggtgcgtatatttttggtaaaaatactgtatggcctctttcaagttgtagcgtacggctatagcgtaccagtatttgagtaatagtgtattgagcgaaaactcattatttagcagatcggttttttctattatctcccttaactcccaaaaaatttctatcctcattttcaggttatttaccttttttaaaagtagattatcggaaaacacctcatgacataagataatgttactactggttttatgaaactttaaatctatgaaaatttgtaaaatatccccttcatttaaggtcttcttggcgcctagctctcgacagaggtcccaggtgtgctccgtgttgacagatatcagcccgtagttgatgttcgctccccactccgtaaatagttttataagactgtagttgttttcctttacagccttcactaatgccatgtttaggtctaagccttcttgaaggcctgctgattttatcagccttaggttatgatcaaacgtgatcggagcattattccaccatagatcgtaacactttaaaagataatgttggtcctcgggcagacattgtccagccagcacctttttggtcagagattgcagggaaggcaacat

SEQ ID NO: 99 - DNA, 1056 bp, African swine fever virus
tcatcttagatcataagtaaggaagaccatcatattttttgaaaagtaattttttaacgcatgatctataatttcagggtccgtgcttttaggcaatggggtagtggccgggctataaatctttagggataaaatattccttataggctcatacccttcctgtcccactagagccataccctcttcaaaagcatcggcccccagatccatacaaaagaatatgttttctatattatagtactgtattgaggtaagcatggcctgattgatgttggcgcccagcatatagcagtagtatattgttgaaaggttgtggtctttgatgcaggctatccgcatcatctcttctaaatccatacagatcttgtccttttcatacgcctcatgaaggtcaaacacgttattaaaacaaagagcacatgttaaccgccacgtattcaggtgtgtatatttttggtaaaaatactgtatggcctctttcaagttgtagcgtacggctatagcgtaccagtatttgagtaatagtgtattgagcgaaaactcattatttagcagatcggttttttctattatctcccttaactcccaaaaaatttctatcctcattttcaggttatttaccctttttaaaagtaaattatcggaaaacacctcatgacataagataatgttactactggttttataaaattttaaatctatgaaaatttgtaaaatatccccttcatttaaggtcttcttggcgcctagctctcgacagaggtcccaggtgtgctccgtgttgacagatatcagcccgtagttaatgttcgccccccactccgtaaatagttttataagactgtagttgttttcctttacagccttcactaatgccatgtttaggtctaagccttcttgaatgcctgctaattttatcagccttaggttatgatcaaacgtgatcggagcattattccaccatagatcgtaacactttaaaagataatgttggtcctcgggcagacattgtctagccagcacctttttggtcaaagattgcagggaaggcaacat

SEQ ID NO: 100 - DNA, 1062 bp, African swine fever virus
ctatagtatattatgagaatattcccaaatgatggataagttttttgatttataatcttttaataaactgcttatttcttcggggtcctttaagtttagtggcaaggaagcatctgagctgtaaatatccaaagccaaactatggctcagaaaattataacctttttgttccgctatggcacgaccctcttcaaaggcattaccacccaaatctatacagaaaaatatattaccgatgttataatattgtactgaagtaagcatagcttggttgatgttgccccccagcgcgtaacagtaatatattgttaatggattgttatccttggtagaagccagacatatcatgtcatggacgtctatttggatgttttccttgtggtacatctcatgaagctcatatattttgttataatacaggagacattttaatcgccattcattaagatccgtatatttctcatctagaaaacaaatggcgtccttacaatcgtattgtactgctttggcgtaccaatacttcactagtaaaccatttaactcgtccgtttcttttatttctatgagcccccatagtcttttataaattaagccccttaattgtataacaaatttgttttctaaaataggattattcataaaaatttcatggcacaaaataatactgccgctggttttattgtgcattatcctggtaaaaatacggaaaatatcgttgtcctctagagtttctttggcgcctagctgtctacacaactctcggatgtgcttcgtattgatagaaagcaaaccatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacaatatttcaaaatgcagtgttcatctattgacaaacactggctggctatcgtctttttgacgagggtctgcagagagagcggcaacgacat

SEQ ID NO: 101 - DNA, 1062 bp, African swine fever virus; identical to SEQ ID NO: 100

SEQ ID NO: 102 - DNA, 1062 bp, African swine fever virus
ctatagtatattatgagaatattcccaaatgatggataagttttttgatttataatcttttaataaactgcttatttcttcggggtcctttaagtttagtggcaaggaagcatctgagctgtaaatatccaaagccaaactatggctcagaaaattataacctttttgttccgctatggcacgaccctcttcaaaggcattaccacccaaatctatacagaaaaatatattaccgatgttataatattgtactgaagtaagcatagcttggttgatgttgccccccagcgcgtaacagtaatatattgttaatgggttgttatccttggtagaagccagacatatcatgtcatggacgtctatttggatgttttccttgtggtacatctcatgaagctcatatattttgttataatacaggagacattttaatcgccattcattaagatccgtatatttctcatctagaaagcaaatggcgtccttacactcgtattgtactgctttggcgtaccaatacttcactagtaaaccgtttaactcatccgtttcttttatttctatgagcccccatagtcttttataaattaagccccttaattgtatagtaaatttatcttctatattaggattattcataaatatatcatggcacaaaataatactgccgctggttttattgtgcattatcttggtgaaaatacggaaaatatcgtcgtcttctagagtttctttggcgcctagctgtctacacaactctcggatgtgcttcgtattgatagaaagcaaactatagttgatatttgcgccccactctgtaaagagctttatcagactatagttgttttccttaacagctattattaatgctatatggaggtctatatcttctcctaaaaatcctgattttatttgtatttggccacgatccatacaaagcttgagggggacatcatgccaccataggccacagtgtttcagaatgcagtgttcatctatcgacaaagactggttggctaccgtctttttgacgagggtctgcagagagagcggtaatgacat

SEQ ID NO: 103 - DNA, 1062 bp, African swine fever virus
ctatagtatattatgagaatattcccaaatgatggataagttttttgatttataatcctttaataaactgcttatttcttcggggtcctttaagtttagtggcaaggaagcatctgagctgtaaatatccaaagtcaaactatggcttagaaaatgataacctttttgttccgctatagcacgaccctcttcaaaggcattaccacccaaatctatacagaaaaatatattaccgatgttataatattgtactgaagtaagcatagcttggttgatgttgccccccagggcgtaacaataatatattgttaatggattgttatccttggtagaagccagacatatcatgtcatggacgtctatttggactttttccttgtggtacatctcatgaagctcatatattttgttataatacaggagacattttaatcgccattcattaagatccgtatatttctcatctagaaagcaaatggcgtccttacactcgtattgtactgctttggcgtaccaatacttcactagtaaatcgtttaactcatccgtttcttttatttctatgagcccccatagtcttttacaaattaagccccttaattgtatagtaaatttgttttctatattaggattattcataaatatctcatggcacaaaataatacggccgctggttttattgtgcattatcttggtgaaaatacggaaaatatcgttgtcctctagagtttctttggcacctagctgtctacacaactctcggatgtgctccgtattgatagaaagcaaactatagttgatatgtgcgccccattctgtaaagagctttatcagactatagttgttttccttaacagctattattaatgctatatggaggtctatatcttctcctaaaaatcctgattttatttgtatttggccacgatccatacaaagcttgagggggacatcatgccaccataggccacagtgtttcagaatgcagtgttcatctgtcgacaaacactggctggctaccgtctttttgacgagggtctgcagagagagcggcaacgacat

SEQ ID NO: 104 - DNA, 1062 bp, African swine fever virus
ctatagtatattatgagaatattcccaaatgatggataagttttttgatttataaccctttaataaactgcttatttcttcggggtcctttaagtttagtggcaaggaagcatctgagctgtaaatatccaaagccaaactatggcttagaaaatgataacctttttgttccgctatggcacgaccctcttcaaaggcattaccacccaaatctatacagaaaaatatattaccgatgttataatattgtactgaagtaagcatagcttggttgatgttgccccccagggcgtaacagtaatatattgttaatggattgttatccttggtagaagccagacatatcatgtcatggacgtctatttggactttatccttgtggtacatctcatgaagctcatatattttgttataatacaggagacattttaatcgccattcattaagacccgtatatttctcatctagaaagcaaatggcgtccttacactcgtattgtactgctttggcgtaccaatacttcactagtaaatcgtttaactcatccgtttcttttatttctatgagcctccatagtcttttacaaattaagccccttaattgtatagtaaatttgttttctatattaggattattcataaatatctcatggcacaaaataatacggccgctggttttattgtgcattatcttggtgaaaatacggaaaatatcgttgtcctctagagtttttttggcacctagctgtctacacaactctcggatgtgttccgtattgatagaaagcaaactatagttgatatgtgcgccccattctgtaaagagctttatcagactatagttgttttccttaacagctattattaatgctacatggagatctatatcttctcctaaaaatcctgattttatttgtatttggccacgatccatacaaagcttgagggggacatcatgccaccataggccacagtgtttcagaatgcagtgttcatctgtcgacaaacactggctggctaccgtttttttgacgagggtctgcagagagagcggcaacgacat

SEQ ID NO: 105 - DNA, 1062 bp, African swine fever virus
ctatagtatattatgagaatattcccaaattatggataagttttttgatttataatcctttaaaaaactgcttatttcttcggggtcctttaagtttagtggcaaggaagcatctgagctgaaaatatccaaagccaaactatggcttagaaaattataacctttttgttccgctatggcacgaccctcttcaaaggcattaccacccaaatctatacagaaaaatatattaccgatgttataatattgtattgaagtaagcatagcttggttgatgttgccccccagggcgtaacagtaatatattgttaatggattgttatccttggtagaagccagacatatcatgtcatggacgtctatttggactttttccttgtggtacatctcatgaagctcatatattttgttaaaatacaggagacattttaatcgccattcattaagatccgtatatttctcttctagaaagcaaatggcgtccttacactcgtattgtactgctttggcgtaccaatacttcactagtaaatcgtttaactcatccgtttcttttatttctatgagcccccatagtcttttacaaattaagccccttaattgtatagtaaatttgttttctatattaggattattcataaatatatcatggcacaaaataatacggccgctggttttattgtgcattatcttggtgaaaatacggaaaacatcgtcgtcttctagagtttctttggcgcctagctgtctacacaactctcggatgtgctccgtattaatagaaagcaaactatagttgatatgtgcgccccattctgtaaagagctttatcagactatagttgttttccttaacagctattattaatgctatatggaggtctatatcctctcctaaaaatcctgattttatttgtatttggccacgatccatacaaagcttgagggggacatcatgccaccataggccacagtgtttcagaatgcagtgttcatctatcgacaaacactggctggctaccgtctttttgacgagggtctgcagagagagcggcaacgacat

SEQ ID NO: 106 - DNA, 1062 bp, African swine fever virus
ctataatatattatgagaatattcccaaatgatggataagttttttgatttataatcttttaataaactgcttatttcttcggggtcctttaagtttaatggcaaggaagcatctgaactgtaaatatccaaagtcaagctatggcttaaaaaattataacctttttgttccgctatggcacgaccctcttcaaaggcattaccccccaaatctatacagaaaaatatattaccgatgttataatattgtactgaagtgagcatagcttggttgatgttgctccccagggcgtaacagtaatatattgttaatagattgttatccttggcacaagccagacatatcatgtcatggacgtctatttggacttttttcttgtggtacatctcatgaagctcatatattttgttataatacaggagacattttaatcgccattcgttaagatccgtatatttctcatctagaaagcaaatggcgttcttacaaacgtattgtactgctttggcataccaatacttcactagtaaatcatttaacttgtccgtttcttttatttctatgagcccccatagtcttttataaattaagccccttaattgtataacaaatttgttttctaaaataggattattcataaaaatttcatggcacaaaataatactgccgctggttttattgtgcattatcctggtgaaaatacggaaaatatcgttgtcctctagagtttctttggcgcctagctgtctacacaactctcggatgtgcttcgtattgatagaaagcaaactatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacagtatttcaaaatgcagtgttcatctattgacaaacactggctggctaccgtctttttgacgagggtctgcagagagagcggcaacgacat

SEQ ID NO: 107 - DNA, 1062 bp, African swine fever virus
ctataatatattatgagaatattcccaaatgatggataagttttttgatttataatcttttaataaactgcttattttttcggggtcctttaagtttaatggcaaggaagcatctgaactgtaaatatccaaagtcaagctatggcttaaaaaattataacctttttgttccgctatggcacgaccctcttcaaaggcattaccacccaaatctatacagaaaaatatattaccgacgttataatattgtactgaagtgagcatagcttggttgatgttgccccccagggcgtaacagtaatatattgttaatagattgttatccttggcacaagccagacatatcatgtcatggacgtctatttggacttttttcttgtggtacatctcatgaagctcatatattttgttataatacaggagacattttaatcgccactcgttaagatccgtatatttctcatctagaaagcaaatggcgttcttacaaacgtattgtactgctttggcataccaatacttcactagtaaatcatttaactcgtccgtttcttttatttctatgagcccccatagtcttttataaattaagccccttaattgtataacaaatttgttttctaacataggattattcataaaaatttcatggcacaaaataatactgccgctggttttattgtgcattatcctggtgaaaatacggaaaatatcgttgtcctctagagtttctttggcgcctagctgtctacacaactctcggatgtgcttcgtattgatagaaagcaaactatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacagtatttcaaaatgcagtgttcatctattgacaaacactggctggctaccgtctttttgacgagggtctgcagagagagcggcaacgacat

SEQ ID NO: 108 - DNA, 1062 bp, African swine fever virus
ctataatatattatgagaatattcccaaatgatggataagttttttgatttataatcttttaataaactgcttattttttcggggtcctttaagtttaatggcaaggaagcatctgaactgtaaatatccaaagtcaagctatggcttaaaaaattataacctttttgttccgctatggcacgaccctcttcaaaggcattaccacccaaatctatacagaaaaatatattaccgacgttataatattgtactgaagtgagcatagcttggttgatgttgccccccagggcgtaacagtaatatattgttaatagattgttatccttggcacaagccagacatatcatgtcatggacgtctatttggacttttttcttgtggtacatctcatgaagctcatatattttgttataatacaggagacattttaatcgccactcgttaagatccgtatatttctcatctagaaagcaaatggcgttcttacaaacgtattgtactgctttggcataccaatacttcactagtaaatcatttaactcgtccgtttcttttatttctatgagcccccatagtcttttataaattaagccccttaattgtataacaaatttgttttctaacataggattattcataaaaatttcatggcacaaaataatactgccgctggttttattgtgcattatcctggtgaaaatacggaaaatatcgttgtcctctagagtttctttggcgcctagctgtctacacaactctcggatgtgcttcgtattgatagaaagcaaactatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacaatatttcaaaatgcagtgttcatctattgacaaacactggctggctaccgtctttttgacgagggtctgcagagagagcggcaacgacat

SEQ ID NO: 109 - DNA, 1062 bp, African swine fever virus
ttagagtatattatgagagtaatccaaaatgatggataaattttttgatttataattttttaacaaactgcttatttcttcggggtcctttaagtttaatggcaaggaagcatccgagctataaatattcaaaaccaaactatggcttagaaaattataacctttttgtctcgctatggcactaccctcttcaaaggcattaccacccaaatctatacagaaatatatattaccgatgttataatattgcactgaagtaagcatagcttggttgatgttgccccccagggcataacagtaatatattgttaatggattgttatccctgatacaagccagggataacatttcattaacgtctatttggattttttccttgtggtatatatcatgaagctcatatattttgttataatataggtaacattttaatcgccattcattaagatccgtatatttctcatccagaaagcaaatggcatctttataatcgtattgtactgctttggcgtaccaatatttcactagtaattcacttaactcgtccgtttcttttatttctataagcccgcatagtcttttataaattaagcgccttaattgtgcagcaaatttgttttctaaattaggattatttataaatatctcatggcacaaaataacactgccgctggttttattatgcattatcttgttgaaaatacagaaaatatcgtagtcctccagagtttctttggcgcctagctgtctacacaaatctcggacgtgcttcgtattgatagaaagcagactatagttgatatttgcgcccaactctgtaaaaagctttattagattatagttgttttccttaacagctattattaatgccacacaaaggtctatatcttctcctaaaaatcccgattttatttgtattcggccacgatccatataaagcttgagaggggcatcatgccaccataagtcacagtatttcaaaatgtgttcatctatcgacaaacatgatgtactggctaccgtctttttgacgagggtttgtagagagagcggcgccgacat

SEQ ID NO: 110 - DNA, 1062 bp, African swine fever virus
ttagagtattttatgagagtaatccagaatgatggataaattttttgatttataattttttaataaactgcttatttcttcgggaacctttaagtttaatggcaaggaagcatctgagctataaatattcaaaattaaactatggcataaaaaattataaccattttgtctcgctatggcactaccctcttcaaaggcattaccacccaaatctatacagaaatatatattaccgatgttataatattgtactgaagtaagcatagcttggttgatgttgccccccagggcataacaataatatattgttaatagattgttatccctgatacaagccagagataacatttcattgacgtctatttggatttttttcttgtggtatatatcatgaagctcatatattttgttataacataggtaacattttaatcgccattcatcaagatccgtatatttttcatccagaaagcaaatggcatctttatgatcgtattgtactgcattggcgtaccaatatttcactagtaattcgcttaactcgtccgtttcttttatttctataagcccgcataatcttttataaattaagcgccttaattgtgcagcaaagttgttttctaatttaggattatttataaatatctcatggcacaaaataatactgccgctagttttattatgcattattttattgaaaatacagaaaatatcgtagtcgtctagagtttctttggcgcctagctgtctacacaaatctcgggcgtgcttcgtattgatagaaagcagactatagttgatatatgcgccccactctgtaaataactttatcagactatagttattttccttaacagctattattaatgccacacaaaggtctatatcttctcctaggaatcccgattttatgtatattcgtccacgatctatgtaaagcttgaggggagcatcatgccaccataagccacagtatttcaaaatgtgttcatctatcgacaaacatgatgtactggctaccgtctttttgacgagggtctgcagagagagcggcgacgacat

SEQ ID NO: 111 - DNA, 1062 bp, African swine fever virus
ttagagtattttatgagagtaatccagaatgatggataaattttttgatttataattttttaataaactgcttatttcttcgggatcctttaagtttaatggcaaggaagcatctgagctataaatattcaaaaccaaactatggcttaaaaaattataaccattttgtctcgctatggcactaccctcttcaaaggcattaccacccaaatctatacagaaatatatattaccgatgttataatattgtactgaagtaagcatagcttgattgatgttgccccccagggcataacaataatatattgttaatagattgttatccctgatacaagccagagataacatttcattgacgtctatttggattttttccttgtggtatatatcatgaagctcatatattttgttataacataggtaacattttaatcaccattcatcaagatccgtatatttttcatccagaaagcaaatggcatctttatggtcgtattgtactgcattggcgtaccaatatttcactagcaattcgcttaactcgtccgtttcttttatttctataagcccgcatagtcttttataaattaagcgccttaattgtgcagcaaagttgttttctaaattaggattatttataaatatctcatggcacaaaataatactgccgctagttttattatgcattatcttgttgaaaatacagaaaatatcgtagtcgtctagagtttctttggcgcctagctgtctacacaaatctcgggcgtgcttcgtattgatagaaagcagactatagttgatatatgcgccccactctgtaaataactttatcatactatagttgttttccttaacagctattattaatgccacacaaaggtctatatcttctcctaggaatcccgattttatgtatattcgcccacgatctatgtaaagcttgaggggagcatcatgccaccataagccacagtatttcaaaatgtgttcatctatcgacaaacatgatgtaccggctaccgtctttttgacgagggtctgcagagagagcggcgacgacat

SEQ ID NO: 112 - DNA, 1074 bp, African swine fever virus
ttagtctataactataatttctggatgggctgtaagatactcttcggctcgtttcagattttttgaagtatatgtctttagcatatcatatatttcctggggttcggttacatctaataccaaggtcacatcacggctgaaaagctgctttactaagaaaatgttgctcaagttatacatataagctttgtgcgcaatgagttgtgccctatcaaaatcggcagcccccaaatcaatacagaaaaacatgtttaaagtattattgttatagatagaaagattcatgccataatcgagactagcccccaacctatgacagtaataaatggccgcgtaatttttttcccgcaagcaagcaaatttcatcatcagattagggctgatgcaaatctctttttcacgacacaactcgtgtatgtcaaaaatgttattaaaataaaggctacaagctacccgccaatagaggtgatttttatgccttttatagaaatagtgaatagcctttgtaaaattatgtcgtaatgccagggcaaaccaaaactttgttaataggtggtgcgccgtatcccccgtcaacggaatgtttgaacaggtgtacgtaactgtgtctaaagtggttctagttacggtttccaagagtggattatgacaaaacatgtcataacccagcagaactcctgcacaggattttagcctggccacttcttttaaaatttccagaagacggggttcggatacaggcgttaagcctcccagttccgcacacagccgctttagatacacggcaggaacacgtataagcccatattcaggatttgcgccccaatccacaaataaacgtataagttcaagattatcgctcttcacggcctttactagcgccgcttcgagacaaagatcatcctcagaaaaacactgtaaatgtttatacgaaaaaacttgcttacaattgttacataggtgaataggacctaaatcccaccacaaaccaaaacgctgcaacgtataatcatagtcacttgaaagataattgcatgccacaacttttttggccaacgtttgtaaagacaacat

SEQ ID NO: 113 - DNA, 1074 bp, African swine fever virus; identical to SEQ ID NO: 112

SEQ ID NO: 114 - DNA, 1074 bp, African swine fever virus
ttagtctataactataatttctggatgggctgtaagatactcttcagctcgtttcagattttttgaagtatatgactttagcatatcatatatttcctggggttcggttacatctaatgccaaggtcacatcacggctgaaaagctgctttactaagaaaatgttgctcaagttatacatataagctttgtgcgcaatgcgttgtgccctatcaaaatcagtagcccccaaatcgatgcagaaaaacatgtttaaagtattattgttatagatagaaagattcatgccataatcgagactagcccctaacctataacagtaataaatggccgcataattttttttctgcaagcaagcaaatttcatcatcagattagggctgatgcaaatctctttttcacgacacaactcgtgtatgtcaaaaatgttattaaaataaaggctacaagctacccgccaatagaggtgatttttatgccttttatagaaatagtgaatagcctttgtaaaattatgtcgtaatgccagggcaaaccaaaactttgttaataggtggtgcgccgtatcccccgtcaacggaatgtttgaacaggtgtacgtaactgtgtctaaagtggttctagttacggtttccaagagtggattatgacaaaacatgtcataacccagcagaactcctgcacaggattttagcctggccacttcttttaaaatttccagaagacgggattcggatacaggcgttaagccacccagttccgtgcacagccgctttagatgcacggcaggaacacgtataagtccatattcaggatttgcgccccaatccacaaataaacgtataagctcaagattatcgctcttcacggcctttactagcgccgcttcaagacaaagatcatcctcagaaaaacactgtacatgtttatacgaaaaaatttgcttacaattattacataggtgaataggacctaaatcccaccacaaaccaaaacgctgcaacgtataatcatagtcacttgaaagataattgcatgccacaacctttttggccaatgtttgtaaagacaacat

SEQ ID NO: 115 - DNA, 1074 bp, African swine fever virus
ttagtctataactataatttctggatgggctgtaagatactcttcggctcgtttcagattttttgaagtatatgactttagcatatcatatatttcctggggttcggttacatctaatgccaaggtcacatcacggctgaaaagctgctttactaaaaaaatgttgctcaagttatacatataagttttgtgcgcaatgcgttgtgccctatcaaaatcagtagcccccaaatcgatgcagaaaaacatgtttaaagtattattgttatagatagaaagattcatgccataatcgagactagcccctaacctataacagtaataaatggccgcataattttttttccgcaagcaagcaaatttcatcatcagattagggctgatacaaatctctttttcacgacacagctcgtgtatgtcaaaaatgttattaaaataaaggctacaagctacccgccaatagaggtgatttttatgccttttataaaaatagtgaatagcctttgtaaaattatgtcgtaatgccagggcaaaccaaaactttgttaataggtggtgcgccgtatcccccgtcaacggaatgtttgaacaggtgtacgtaactgtgtctaaagtggttctagttacggtttccaagagtggattatgacaaaacatgtcataacccagtagaactcctgcacaggattttagcttggccacttcttttaaaatttccagaagacggggttcggatacaggcgttaagcctcccagttccgtgcacagccgctttagatgcacggcaggaacacgtataagtccatattcaggatttgcgccccaatccacaaataaacgtataagctcaagattatcgctcttcacggcctttactagcgccgcttcaagacaaagatcatcctcagaaaaacactgtaaatgtttatacgaaaaaatttgcttacaattgttacataggtgaataggacctaaatcccaccacaaaccaaaacgctgcaacatataatcatagtcacttgaaagataattgcatgccacaacctttttggccaacgtttgtaaagacaacat

SEQ ID NO: 116 - DNA, 1074 bp, African swine fever virus
ttagtctataactataatttctggatgggctgtaagatactcttcggctcgtttcatattttttgaagtatatgcctttagcctatcatatatttcctggggttcggttacatctaatgccaaggtcacatcacggctgaaaagctgctttactaaaagaatgttgctcaagttatacatataagctttgtgcgcaatgcgttgtgccctatcaaaatcagtagcccccaaatcgatgcagaaaaacaggtttaaagaattattgttatagatagaaagattcatgccataatcgagactagctcccaacctataacagtaataaatggccgcgtaatttttttcccgcaagcaagcaaatttcatcatcagattagggctaatgcaaatctctttttcacgacacagctcgtgtatgtcaaaaatgttattaaaataaaggctacaagctacccgccaatagaggtgatttttatgccttttatagaaatagtgaatagcctttgtaaaattatgtcgtaatgccagcgcaaaccaaaactttgttaataggtggtgcgccgtatcccccatcaacggaatttttgaacagctgtacataactgtgtctaaagtggttctagttatggtttccaagagtgggttataacaaaacatgtcataacccagtagaactcctgcacaggattttaggttggccacttcttttaaaatttccagaagacggggttcggatacaggtgttaagcctcccagttctgtgcacagccgctttagatgcacggtaggaacacatataagtccatattcaggatttgcaccccaatccacaaataaacgtataagctcaagattatccctcttcacggcctttactagcgccgcttcaagacagagatcatcctcagaaaaacactgtaaatgtttatacgaaaaaatttgcttacaattgttacacaggtgaataggacctaaatcccaccacaaaccaaaacgctgcaacatataatcatagtcacttgaaagataattgcatgccacaacctttttggccaacgtttgtaaagacaacat

SEQ ID NO: 117 - DNA, 1074 bp, African swine fever virus
ttagtctataactataatttctggatgggctgtaagatactcttcggctcgtttcagattttttgaagtatatgactttagcctatcatatatttcctggggttcggttacatctaatgccaaggtcacatcacggctgaaaagctgctttactaagaaaatgttgctcaagttatacatataagctttgtgcgcaatgtgttgtgccctatcaaaatcagtagcccccaaatcgatgcagaaaaacatgtttaaagtattattgttatagatagaaagattcatgccataatcgagactagcccctaacctataacagtaataaatggccgcataattttttttctgcaagcaagcaaatttcatcatcagattagggctgatgcaaatctctttttcacgacacagctcgtgtatgtcaaaaatgttattaaaataaaggctacaagctacccgccaatagaggtgatttttatgccttttatagaaatagtgaatagcctttgtaaaattatgtcgtaatgccagggcaaaccaaaactttgttaataggaggtgcgccgtatcccccgtcaacggaatgtttgaacaggtgtacgtaactgtgtctaaagtggttctagttacggtttccaagagtggattatgacaaaacatgtcataacccagtagaactcctgcacaggattttagcttggccacttcttttaaaatttccagaagacggggttcggttacaggcgttaagcctcccagttccgtgcacagccgctttagatgcacggcaggaacacgtataagtccatattcaggatttgcgccccaatccacaaataaacgtataagctcaagattatcgctcttcacggcctttactagcgccgcttcaagacaaagatcatcctcagaaaaacactgtaaatgtttatacgaaaaaatttgcttacaattgttacacaggtgaataggacctaaatcccaccacaaaccaaaacgctgcaacgtataatcatagtcacttgaaagataattgcatgccacaacctttttggccaacgtttgtaaagacaacat

SEQ ID NO: 118 - DNA, 1074 bp, African swine fever virus
ttagtctataactataatttctggatgggctgtaagatactcttcggcccgtttcatattttttgaagtatatgtctttagcatatcatatatttcctggggttcggttacatctaataccaaggtcacatcacggctgaaaagctgctttactaagaaaatgttgctcaagttatacatataagctttgtgcgcaatgagttgtgccctatcaaaatcggcagcccccaaatcaatacagaaaaacatgtttaaagtattattgttatagatagaaagattcatgccataatcgagactagcccccaacctatgacagtaataaatggccgcgtaatttttttcccgcaagcaagcaaatttcatcatcagattagggctaatgcaaatctctttttcacgacacagctcgtgtatgtcaaaaatgttattaaaataaaggctacaagctacccgccaatagagctgatttttatgccttttataaaaatagtggatagcctttgtaaaattatgtcgtaatgccagggcaaaccaaaactttgttaataggaggtgcgccgtatcccccgtcaacggaatgtttgaacaggtgtacgtaactgtgtctaaagtggttctagttacggtttccaagagtggattatgacaaaacatgtcataacccagtagaactcctgcacaggattttaggttggccacttcttttaaaatttccagaagacggggttcggatacaggcgttaagcctcccagttccgcacacagccgctttagatacacggcaggaacacgtataagcccatattcaggatttgcgccccaatccacaaataaacgtataagttcaagattatcgctcttcacggcctttactagcgccgcttcaagacaaagatcatcctcagaaaaacactgtaaatgtttatacgaaaaaacttgcttacaattgttacataggtgaataggacctaaatcccaccacaaaccaaaacgctgcaacgtataatcatagtcacttgaaagataattgcatgccacaacttttttggccaacgtttgtaaagacaacat

SEQ ID NO: 119 - DNA, 1074 bp, African swine fever virus
ttagtctataactataatttctggatgggctgtaagatactcttcggctcgtttcatattttttgaagtatatgtctttagcatatcatatatttcctggggttcggttacatctaataccaaggtcacatcacggctgaaaagctgctttactaagaaaatgttgctcaagttatacatataagctttgtgcgcaatgagttgtgccctatcaaaatcggcggcccccaaatcaatacagaaaaacatgtttaaagtattattgttatagatagaaagattcatgccataatcgagactagcccccaacctatgacagtaataaatggccgcgtaatttttttcccgcaagcaagcaaatttcatcatcagattagggctaatgcaaatctctttttcacgacacagctcgtgtatgtcaaaaatgttattaaaataaaggctacaagctacccgccaatagagctgatttttatgccttttataaaaatagtggatagcctttgtaaaattatgtcgtaatgccagggcaaaccaaaactttgttaataggaggtgcgccgtatcccccgtcaacggaatgtttgaacaggtgtacgtaactgtgtctaaagtggttctagttacggtttccaagagtggattatgacaaaacatgtcataacccagtagaactcctgcacaggattttaggttggccacttcttttaaaatttccagaagacggggttcggatacaggcgtcaagcctcccagttccgcacacagccgctttagatacacggcaggaacacgtataagcccatattcaggatttgcgccccaatccacaaataaacgtataagttcaagattatcgctcttcacggcctttactagcgccgcttcaagacaaagatcatcctcagaaaaacactgtaaatgtttatacgaaaaaacttgcttacaattgttacataggtgaataggacctaaatcccaccacaaaccaaaacgctgcaacgtataatcatagtcacttgaaagataattgcatgccacaacttttttggccaacgtttgtaaagacaacat

SEQ ID NO: 120 - DNA, 1074 bp, African swine fever virus; identical to SEQ ID NO: 119

SEQ ID NO: 121 - DNA, 1074 bp, African swine fever virus
ttagtctataactacaatttctggatgggctgtaaaatactcttcggcttgttttagattttttgacgtatatgtatttagcatatcatatattgcctggggctcagttagatctaatatcaagctcacatcacggctgaaaagctgttttactaaaaaaaggttgctcaagttatacatataaactctacgcgcaatgagtcgcgccctatcaaagttagtagcccccaaatcaatgcagaaaaataggtttaaagtattattgttatagatagatagattcatgccataatcaagactagcccctaacatatgacagtaataaatggctgcataattttcttcccgcaagcaagcaaatttcatcatcagattagggctaatgcaaatctcttttttacaacacagctcatgcaagtcaaaaatgttgttaaaataaaggctacaagtcagtcgccaatagagctgatttttatgcttttcgtagaaatagtgaatagcttttgtaaaattatgtcgtaatgccagggcaaaccaaaactttgataataggaggtgcgccgtatcccccgtcaacggaatttttgaacgggtgtacataactgtgtctaaggtggttctagtcacggtttccaagagtggattatgacaaaacatgtcataacccagtagaactcctgcacaggatttcagatcggccacttcttttaaaatctccagaagacgagattcggatacaggtgttaagcctcctagttccatgcacagccgctttagatgcacggcaggaacacgtataagtccatattcaggatttgcgccccaatccacaaacaatcgtataagctcaagattatcgctcttcacggcctttactagcgccgcttcaagacaaagatcatcctcagaaaaacactgtaaatgtttatatgaaaaaatttgcttacatgtattacataagtgaataggacctaaatcccaccacaaaccaaaacgctgcagcgtataatcataatcacttgaaagataattgcatgccacaacttttttggccagcgtttgtaaagacaacat

SEQ ID NO: 122 - DNA, 1074 bp, African swine fever virus
ttagtctataactacaatttctggatgggctgtaaaatactcttcggctcgttttagattttttgacgtatatgtctttagcatatcatatattgcctggggttcggttatatttaataccaagctcgcatcacggctgaaaagctgttttactaaaaaaatgttgctcaagttatacatataaactctatgcgcaatgagtcgcgctctatcaaagttagtagcccccaaatcaatacagaaaaataggtttaaagtattattgttatagatagatagattcatgccataatcgggactagcccccaacatatgacagtaataaatggctgcataattttcttcccgcaggcaagcaaatttcatcattagattagggctaatgcaaatctcttttttagaacacagctcatgcaagtcaaaaatattattaaaataaaggctacaagccagccgccaatagagctgatttttatgcttttcatagaaatagtgaatcgcttttgtaaaattatgtcgtaatgccagggcaaaccaaaacttcgttaataggaggtgcgccgtatcccccgtcaacggaatttttgaacgggtgtacataacggtgtttaaagtggttctagtcacggtttccaagagtggattatgacaaaacatgtcataacccagtagaactcctgcacaggatttcagatcggccacttcttttaaaatttccagaagacgagattcggagacaggtgttaagcctcctagttctgtgcacagccgttttagatccacggcaggaacacgtataagtccatattcaggatttgcgccccaatccacaaacaatcgtataagctcaagattatcgctcttcacggcctttactagcgccgcgtcaagacaaagatcatcctcagaaaaacactgcaaatgttgatacgaaaaaatttgcttacatgtattacataagtgaataggacctaaatcccaccacaaaccaaaacgctgcagcgtataatcatagtcacttgaaagataattgcatgccacaacctttttggccaacgtttgtaaagacaacat

SEQ ID NO: 123 - DNA, 1074 bp, African swine fever virus
ttagtctataacgacaatttctggatgggctgtaaaatactcttcggctcgttttagattttttgacgtatatgtctttagcatatcatatattgcctggggttcggttatatttaataccaagctcgcatcacggctgaaaagctgttttactaaaaaaatgttgctcaagttatacatataaactctatgcgcaatgagtcgcgctctatcaaagttagtagcccccaaatcaatgcagaaaaataggtttaaagtattattgttatagatagatagattcatgccataatcgggactagcccccaacatatgacagtaataaatggctgcataattttcttcccgcaggcaagcaaatttcatcattagattagggctaatgcaaatctcttttttagaacacagctcatgcaagtcaaaaatgttattaaaataaaggctacaagttagccgccaatacagctgatttttatgtttttcgtagaaatagtgaatcgcttttgtaaaattatgccgtaatgccagggcaaaccaaaacttcgttaataggaggtgcgccgtatcccccgtcaacggaatttttgaacgggtatacataactgtgtttaaagtggttctagtcacggtttccataagtggattatgacaaaacatgtcataacccagtagaactcctgcacaggatttcagatcggccacttcttttaaaatttccagaagacgagattcggagacaggtgttaagcctcctagttccgtgcacagccgttttagatgcacggcaggaacacgtataagtccatattcaggatttgcgccccaatccacaaacaatcgtataagctcaagattatcgctcttcacggcctttactagcgccgcgtcaagacaaagatcatccacagaaaaacactgcaaatgttgatacgaaaaaatttgcttacatgtattacataagtgaataggacctaaatcccaccacaaaccaaaacgctgcagcgtataatcatagtcacttgaaagataattgcacgccacaacttttttggccaacgtttgtaaagacaacat

SEQ ID NO: 124 - DNA, 1071 bp, African swine fever virus
atgtctactccactttctctacagactcttgttaaaaaagtgctggccacacagcacatatctaaagaacactactttattttgaaatattgtggtttatggtggcatgaagcgccgattacgatttgcattgatgaggatagccaaatattgataaaatcggcaagcttcaaagaaggcttatctttagatatcgcattaatgaaagtcgtgcaagaaaataaccatgatttaatagagttgtttaccaagtggggtgcagatatcaactctagcttagttactgttaatacggagtatacccggaacctttgtcagaaattaggcgcaaaggaagctttgaatgaaagggatattttacaaatattttataaaacacgtcatcttaaaactagcagtaatattattttatataatgaattgttttctaataatctccttttccaaaatatagagagattgagtttaatagtttataggggcttgaaaaacttatcaatcaactttatattggatgatatttcatttagcgaaatgttaactagatactggtatagtatggcgatattatataaccttactgaagccatccaatatttttatcaacgatataggcattttaaagattggcggcttatatgtgggctttcttttaacaatttgtctgaccttcatgaagtatataacttagagaagacggatatagacattgatgaaatgatgaagttgacctgtagtacgtatgatggtaattattcgactatttattattgttttatgttgggggctgacatcaatcgggcaatgttaacctcggtaataaactttcatattggtaacttgttcctttgtatagatttaggagctgatgctttcgaagacagcatggaactagcaaaacaaaagaataataatatattagtagaaatattatcatttaaaaattattatagttcaaatacctctcttttatcaataaaaacgacagatccggaaaaaattaatgccttattagatgaagaaaagtatgagtcaaaaaatatgttaatgtatgaagaattatctcattga

SEQ ID NO: 125 - DNA, 1071 bp, African swine fever virus; identical to SEQ ID NO: 124

SEQ ID NO: 126 - DNA, 1065 bp, African swine fever virus
atgtctactccactttctctacagactcttgttaaaaaagtgctggccacacagcacgtatctaaagatcactactttattttaaaatattgtggtttgtggtggcataaggccccgattacgatttgcattgatgaggatagccaaatattgataaaatcggcaagctttaaagaaggcttacctttagatatggcattaatgaaagctgtacaggaaaacaactatgaaatgatcatgttgtttactgagtggggtgcagacattaactctagcttagtcactgttaatacggagcgtacccggaacctttgtcgagaattaggcgcgaaggaaattttgaatgaaaaagaaattttagaaatattttataaaacatgtcatattaaaactagcagtaattttattctatgccatgaattgttatccaataatccccttttccaaaatatagagagtttgagtttaatagtttataggggcttgaaaaacttatcaattaactttatattggatgatatttcatttagcgaaatgttaactagatactggtatagctgggcgatattatataaccttactgaagctattcaatatttctatcaaacatacaagcattttaaagattggcggctaatatgtggacttgcttataataacgtgtttgatcttcatgaaatttataacatagaaaagactgatttagatattgatgaaatgatgcagttggcttgtatgtatgatagtaattattcaactatttattattgttttatgttgggggctaatatcaatcaggcaatgttaacctcggtaataaaccattatattaataacttgttcctttgtatagatttgggagctgacgccgtcaaagacagcatggaactagcaaaacaaaagaattataatatattagtaaaaatattaacatttaaaaattatagtccagactcttctcttttatcattaaaaacgacagatccggaaaaaattaatgccttactagatgaagaaaagtatgagtcgaaaaatatgttaatgtatgaagaatcatctcgttga

SEQ ID NO: 127 - DNA, 1052 bp, African swine fever virus
atgccttctccacattctctacagactcttgctaaaaaaatactggctacacagcagatatctacagatcactactttattttaaaatattgtggtttatggtggcatggagctccaattatgctttctactaatgaggataatcaattgatgataaaatcggcaagcttcaaagaaggcttatctttagatttggcattaatgaaagtcgtgcaagaaaacaatcatgatttaataaagctgtttaccgagtggggtgcagacatcaactctagcttcgtcactgttaatatggagtgtacccgaaacctctgtcgggaattaggcgcgaaggaagctttgaatgaaagggatattttacaaatattttataaaacacgtgatattaaaactagcagtcatgttattttatgccatgaattattatccaataacccccttttccagaatatagagagaatgcgttcaataatttataggagtctggaaaaactatcaatcaactttatattggatgatatttcatttagtgaaatgttaactagacactggtatgggttggcgatattatataacctaactgaagctatccaatatttctatgaaaaatacaagcattttaaaaattggcggctaatatgtggactttcttttaacaatctgtctgatctttatgaaatatataatttagagaaggttgatatgaacattgatgaaatgatgtatctggcctgtagtatatatgatggtaattattcaactatttattattgttttgtattgggagctgatatcaatcaggcaatgttaacctcagtaataaaccattgtattggtaacttgttcctttgtatagatttaggggctgatgctttcgaagacagcatggaactagcaaagcaaaaaaatgataatatttttattagtatattatcatttaaaaattacagtccagactcttctcttttgtcattaaaaatgacagatccggaaaaaattaatgccttactagatgaagaaaagtatgagtcaaaaaatatgttaatgtttga

SEQ ID NO: 128 - DNA, 1086 bp, African swine fever virus
atgcctactccactttctctacagactcttgctaaaaaagtactggccacacagtatatatccatagattattactttattttgaaatattgtggtttatggtggcatggagcccctattatgatttccattgataagaataaaagccaaatactgataaaatcggcaagcttcaaagaaggcttatctttagatatcgcattaatgaaagctgtgcaggaaaacaatcatgatttaataaagctgtttaccgagtggggtgcagacatcaactctagcttagtcactgttaatacggagtgtacccgaaacctctgtcgggaattaggcgcgaaggaagctttgaatgaaagggatattttacaaatattttataaaacacgtgatattaaaactagcagtaatgttatactatgccatgaattgttatccaataacccccttttccaaaatatagagagaatgcgttcaataatttataggagtctggaaaaactatcaattaactttatattggatgatatttcatttagcgaaatgttaactagacactggtataggttggcgatattatataacctaactgaagctatccaatatttctatgaaaaatacaagcattttaaagattggcggctaatatgtgggctttcttttaacaatctgtctgaccttcatgaaatatataacttagagaaggttgatatggacattgatataatgatgtatctggcctgtagtacgtatgatggtaattattcaactatttattattgttttgtattgggggctgatatcaatcaggcaatgttaacctcggtaataaaccatcgtattggtaacttgttcctttgtatagatttaggggctgatgccttcgaagacagtatgaaactagcaaaacaaaagaatcataatatattagtacatatattatcgtttaaaaactatagtccagacttctcccttttatcaataaaaacgacagatccagagaaaattaatgccttactagatgaagaaaagtatgagtcaaaaaatatgttaatgtatgatgaatttgacgcctgtactaataatgtttaa

SEQ ID NO: 129 - DNA, 1086 bp, African swine fever virus; identical to SEQ ID NO: 128

SEQ ID NO: 130 - DNA, 1074 bp, African swine fever virus
atgcctacaccgtcttctttacaagtccttgtcaaaagggtgttggactaccagcatcatgtatctgaagatgattattgtattttacagcattgtgggttgtggtggcatggaagtccaattatgttttttactaatgaaaatcatcaaaggatgataaaatcagcaagctttaaagatggtttagaaataaatctcgcattaatgaaggccgtacaggaaaacaattgtatcttaatagagctgtttaccgaatggggtgcagacattaattatggattgattactgtcaatacggaatatacccgaaacctatgtcgaaatctaggtgccaaggaggcgttaaatacacggaaaattttagatgtatttttaaatttaaaggattttaaaagtagtaatcatattattctatgccatgaattgttatctaataatcctcttttactaagtgaaaataatgattatttgagaaaaataattaattgtaatctaaggagaatatcaattaactttatattggatgaaatttcatttaatgaaaagctaaccagattctggtataaacaggcagtactaaataatcttactgaagctatccaatatttctatcaaaaatataagcagtttaaagactggcgactaatatgtggacttgctttaaataatgtatttgatcttcatgaaatatataacaaagagaaggctgatatagatattaatcaaatgatagagataacctgtacatatatgtgtagttatcccaccatttactattgttttgtaatgggggctgacattaatcgggcaatgattacctcagttacaaaatcttatactggtaacttgttcttttgtatagatttaggagctactgccttcgaagagtgcttagaaatagcaaaacaacagaatgataatgaattggtaaaggtattatcattgaaaaactattacagtccagatagctcccttttatcattaaaaataacagatccggaaaaaattaatatcttattagatgatgaaacatatgagtcaaaaaatgagttaatatatgaagaattaatataa

SEQ ID NO: 131 - DNA, 1095 bp, African swine fever virus
atgtccacaccatcttctttacaagtccttgtcaaaagggtgctggacttccagcatgtgtctgaagatgattactgtattttaaaatgttgtgggttatggtggcatggaggcccaatcatgctttctacgaatgaggataatcaaatgatgataaaatcagcaagttttaaagatggtttagagataaatctcgcattaatgatggccgtacaagaaaacaattgtagcctaatagagctgtttaccgaatggggtgcagacattaattctggattggttactgtcaacacggaatacacccgaaacctatgtcgaaatctaggtgctaaggagacattaaataaacgggaaattttagatgtatttttaaaattaaagaattttaaaagtagtaataatattattctatcccatgaattattatctaataatcctcttttcctaagtgaagataatgattattttagaagaataattaattgtaatctaaggagaatatcaattaactttatattggatgaaatttcatttaatgaaaagctaaccagattctggtatagtcaggcggtactatataatcttactgaagctatccaatatttctatcaaaaatatgaacatctcaatgagtggcgtttaatttgtgcgctttcttttaataatgtgtttgatcttcatgaaatatataacaaagaaaaggttggtatggatattaatcaaatgatagagataacctgtgcatatatgtgtagttattccaccatttactattgttttgtaatgggagctgacattaatcgggcaatgattatctcggttacaaaatcttatacttataacttgttcttttgtatagatttaggagctactgccttcgaagagtgtttagaaatagcaaaacaacagaataataatgaattagtaaaaatattatcattgaaaaactattatagtccagatagctcccttatatcattaaaaataacagatccggagaaaattaatatcttattagatgaggaaaattatgagtcaaaaaatgagttaatatatgaagaatctaatataaataatagcgatgatatattttga

SEQ ID NO: 132 - DNA, 1071 bp, African swine fever virus
atgccatctactttacaagcacttgctaaaaaagtactggccacaccgcatatatctaaaaaatattgtcagtctaaggagtactgtcatatattaaaatgttgtggtttatggtggcatgatgctccaattacaatttatccttgtataaataaaatattgataaaaacagcaagctttaaacatggtttagagttgaatgtcgcattcatgaaagctgtacaggaaaataattatgatctaataaggctgtttattgagtggggtgcggatatcaactttgggttggccactgttaatacagaccgtacccgggacctgtgccgggaactaggtgcgaaggaagctttaagtgaaaaagaaattttagaaatatttgataaaatacagtatattaaaagtagcagtaatattattgtatcccatgaattattatctaataacccccttttcttaaataatgatcaattgaaattaagaatgttcgatgaactacataaactatcaatcaactttgtattacatgagatatcatttaatgaaatgctaactaggtattggtatagtatggcgatactatataacctcaccgcagccattcaatatttttatcaatcctacaagcattttaaagattggcggttaatatgtgggcttgcttataacaacgtgtttgatcttcatgaaatatataacaaagagaaggttgatatggatattaatcaaatgatgcagttggcctgtatgtatgatggtaattatacaaccatttattattgttttatgttgggagctgacattaatcgggcaatgattacctcggttacaaaatcttgtgatggtaacttattcctttgtatagatttgggggctgatgtatttgaagagagtatgaaaatggcaatagaagatcataatgacgtattagaaagtatcttatcatttaaaaattattatagttcagacgtttctctgttatcattaaagacgacagatccagaaaaaattaatgccttgttagaagaagaaagttacaaatcaaaaaataggttgatatataaaagttattga

SEQ ID NO: 133 - DNA, 1074 bp, African swine fever virus
atgatgccatctactttacaaacacttgctaaaaaaacattggccacacagcatatatctaaaaaatattggccgtctgaggagtattgttatatattaaaatgttgtggtttatggtggcatgattctccaatcacgatttttacttgtataaaacaaatattaataaaaacagcaaactttaaacatggtttagacttaaaccttgcattaatgaaagctgtacaagaaaataactatgaattgatcatgttgtttactgagtggggtgcggatatcaacttggggttgatcactgttaatacagagtgcacccgagatttgtgccaaaagttaggtgcaaaggaagctttgagtgcaaaggaaattttagaaatattttataaaatacagtatattaagagtagcaataatattattatatctcatgaattaatatctaatcatccccttttcctaaataatgatcaattgaaattaagaattgttggtgaactaaatgctatatcaatcaactttatattggatgagatatcattcaatgaaatgctaactaggtattggtatagtatggcgatactatataaactccctgcagccattcaatatttttatcaatcatacaagtattttaaagattggcggttaatatgtagccttgcttataacaacgtgtttgaccttcatgaaatttataataaagagaagactgatataaacattgatgaaatgatgcggttggcctgtaggtatgatggtaattatacaactatttattattgttttatgttgggggctgacattaatcaggcaatgattacctcggtcatgaatctttgggacggtaacttatttctttgtatagatttgggggctgatgtatttgaagagtgtatgaaaatagcaatagaagatcataatggtgtattagaaagtatcttatcatttaaaaattattatagtccagacgtttctctgttatcattaaaaacgacagatccggaaaaaattaatgacttgctagatgaagatatctataaatcaaaaaataggttgatatataaaagttgttga

SEQ ID NO: 134 - DNA, 1596 bp, African swine fever virus
atgttctctctccagaacttatgtcgaaaaacattacctaaccgtaaacttcctgaattttttgacgaatatatattacaactgctgggattatactgggaaaaccatggaactattcaacgagcaggaaacaactgtgtgcttatacagcaacataccctcattcccgtaaatgaagccctgagaacagcagcatctgaagaaaattatgagatcgtgagccttttattagcgtgggaggggaacctttactatgctattataggggctctagagggcaaccgccacgacttaattcgtaaatatgatgaccaaatcaaggaccatcatgaaattctgccattcattgacgatccagtcatatttcacaaatgccatatcatgcggcaatgcttttttgattgtattttatatcaagctgtaaaatatagtaagtttcgcgttcttctttactttaaacatagattagaggatgatttgcccttcactcatttacttattgaaaaggcatgtaaagatcataattatgaagttattaaatggatatatgaaaacctacatatctacaatatgatagatacctttgaatgtgctattgcccataaggatctacatctatattgtttggggtatagatttatatataacagaatcgtacccgataagtatcatcatttagatattcgcatgctttcaagcctacaactcctacataaggtggcagccaaaggatacttagattttatcctagaaaccttaaagtatgatcataataaagataatataaatattattctaacacaagctgcaacctataaccatagaaaaattttaatctatttcattcctcaatcaacccacgcacagatagaacaatgtttactagtggcgataaaagcaaaatcttccaggaaaaccttgaacttactactgtctcacctaaacctttccatcaacctcatcaaaaaaataagccattatgttgccacttacaattcaacaaatataataggcattctgagtatgcggcggaaaaagaagatatatttagatatcatattgacaaaatttgtaaaaaaagctatttttaataagtttgtcgttcgatgtatggatacattttctataaacccggaaagaatccttaaaatagccgcgcgaataaataggatgatgttagtgaaaaaaatatctgaacatgtttggaaaaatcatgcggttagacttaaataccttaaacatgcggtacacacgatgaagcataaagatgggaaaaatagactcatgaactttatctatgatcgctgttattaccatatgcaaggggaagaaatctttagcctcgcaagattttatgcaatccatcatgcaccaaagttgtttgacgttttttatgattgttgtatcctagatacgatacgattcaaaagccttcttttagattgttcacatatcataggtaaaaacgctcatgatgctaccaatatcaacatcgtgaacaagtatatcggcaacctgtttgttatgggagttcttagcaaaaaagaaatcttacaggactatccatccatttattctaaacaatacatgccttag

SEQ ID NO: 135 - DNA, 1596 bp, African swine fever virus; identical to SEQ ID NO: 134

SEQ ID NO: 136 - DNA, 1596 bp, African swine fever virus
atgttctctcttcagaacctatgtcgaaaaacattacctgactgtaaacttcctgaattttttgacgaatatatattacaactgctgggattatactgggaaaaccatggaactattcaacgagcaggaaacaactgtgtgcttatacagcaacataacctcattcccgtaaatgaagccctaagaatagcagcatctgaagaaaattatgagatcgtgagtcttttattagcatgggaggggaacctttactatgctattataggggctctagagggcaaccgccacgacttaattcgtaagtatgatgaccaaatcaaggaccatcatgaaattctgccattcattgacgatccagtaatatttcacaaatgccatatcatgcggcaatgcttttttgattgtattttatatcaagctgtaaaatatagtaagtttcgcgttcttctttcttttaaacatagattaagggatgatttgcccttcactcatttacttattgaaaaggcatgtaaagatcataattatgaagtaattaaatggatatatgaaaacctacatatctacaatatgatagatacctttgaatgtgctattgcccataaggatctacgtctatattgtttggggtatagatttatatataacaggatcgtacccgataagtatcatcatttagatattcgcatgctttcaagcctacaactcctgcataaggtggcagccaaaggatacttagattttatcctagaaaccttaaagtatgatcataataaagataatataaatattattctaacacaagctgcaacctataaccatagaaaaattttaatctatttcattcctcaatcaacccacgcacagatagaacaatgtttactagtggcgataaaaacaaaatcttccaagaaaaccttgaacttactactgtctcacctaaacctttccatcaatctcatcaaaaaaataagccattatgttgccacttacaattcaacaaatataataggcattctgagtatgcggcggaaaaagaagatatatttagatatcatattgacaaaatttgtaaaaaaagctatttttaataaatttgtcgttcgatgtatggatacattttctataaacccggaaagaatccttaaaatagccgcgcgaataaataggacgatgttagtgaaaaaaatatctgaacatgtttggaaaaatcatgcggttagacttaaataccttaaacatgcggtacacacgatgaagcataaagatgggaaaaatagactcatgaactttatctatgatcgctgttattaccatatgcaaggggaagaaatctttagcctcgcaagattttatgcaatccatcatgcaccaaagttgtttgacgttttttatgattgttgtatcctagatacgatacgattcaaaagccttcttttagattgttcacatatcataggtaaaaacgctcatgatgctaccaatatcaacatcgtgaacaagtatatcggcaacctgtttgttatgggagttcttagcaaaaaagaaatcttacaggactatccatccatctattctaaacattacatgccttag

SEQ ID NO: 137 - DNA, 1596 bp, African swine fever virus
atgttctctctccagaacctatgtcgaaaaactttacctgactgtaaacttcctgaattttttgacgaatatatattacaactgctgggattatactgggaaaaccatggaactattcaacgagcaggaaacaactgtgtgcttatacagcaacgtaccctcattcccgtaaatgaagccctgagaatagcagcatctgaagaaaattatgagatcgtgggccttttattagcgtgggaggggaacctttactatgctattataggagctctagagggcaaccgccacgacttaattcgtaaatatgatgaccaaatcaaggaccatcatgaaattctgccattcattgacgatccagtcatatttcacaaatgccatataatccgacgatgcttttttaattgtattttatatcaagctgtaaaatatagtaagtttcgcgttcttctttactttaaacatagattagaggatgatttgcccctcactcatttacttattgaaaaggcatgtgaagatcataattatgaagttattaaatggatatatgaaaacctacatacctacgatataatggatacctttgaatgtgctattgcccataaggatctacgtctatattgtttggggtatacatttatatataacaggatcgtaccctatgagtatcatcatttagatattctcattctttcaagcctacaactcctgcataaggtggcagccaaaggatacttagattttatcctagaaaccttaaagtatgatcataacaatgataatttagatattattctaacacaagctgcaacatataaccatagaaaaattttaacctatttcattcctcaattaacctacgcacaaatagaacaatgtttgttcatggcgataaaaagaaaatcttccaagaaaaccttgaacttactactgtctcacctaaccctttccatcgaactcatcaaaaaaataagccaatatgttgtcacttacaattcaacaaatataataggcattctgagtatgaagcggaaaaagaagatatatttagatatcatgttgacaaaatatgtaaaatacgctatttttaataaatatgtcgttcgatgtatggatagattttccataaacccggaaagaatcatcaaaatggccgcgcgaataaataggatgatgttagtgaaaaaaatatctgaacatgtttggaaaaatcatgcggctagacttaaacaccttaagcatgcggtacacacgatgaagcataaagatgggaaaaatagactcatgaactttatctatgatcgctgctattaccatatgcaaggggaagaaatctttagcctcgcaagattttatgcaatccatcatgcaccaaagttgtttgacgttttttatgattgttgtatcctagatactatacaattcaaaagccttcttttagattgttcacatatcataggtaaaaacgctcatgatgctaccaatatcaacatcgtgaacaagtatattggcgacctgtttgctatgggagttcttagcaaaaaagaaatcttacaggactatccatccatctattctaaacattacatgctttag

SEQ ID NO: 138 - DNA, 1596 bp, African swine fever virus
atgttctctctccagaacttatgtcgaaaaacattacctgattgtaaacttcctgaattttttgacgattatatattacaactgctgggattatactgggaaaaccatggaactattcaacgggcaggaaacaactgtgtgcttatacagcaacatacccttattcccataaatgaagccctgagaacagcagcatctgaagaaaattatgagatcgtgagccttttattagcatgggaggggaatctttactatgctattataggggctctagagggcaaccgccacgacttaattcgtaaatatgatgaccaaatcaaggaccatcatgaaattctgccattcattgacgatccagtaatatttcacaaatgccatatcatgcggcgatgcttttttgattgtattttatatcaagctgtaaaatatagtaagtttcgcgttcttctttattttaaatataggttagaggatgatttacccctcactcatttacttattgaaaaggcatgtgaaaatcataattatgaagttattagatggatatatgaaaacctacatatctacaatatgatagatacctttgaatgtgctattgcccataaggatctacgtctatattgtttggggtatacatttatatataacaggatcgtacccaataagtatcatcatatagatattctcattctttcaagcctacaactcctgcataaggtggcagccaaaggatacttagactttatcctagaaaccttaaagtatgatcataacaatgataatttagatattattctaacacaagctgcaacatataaccatagaaaaattttaacctattttattcctcaatcaacctacgcacaaatagaacaatgtttgatggtggcgataaaaacaaaatcttccaagaaaaccttgaatttactattgtcccacctaaacctttccatcaaactcatcaaaaaaataagccaatatgttgtcacttacaattcaacaaatataataggcattctgagtatgaagcgaaaaaagaagatatatttagatatcttgttgacaaaatttgtaaaaaatgctatttttaataaatttgtcgttcgatatatggatacgttttctataaacccggaaaaaatcgtcaaaatggccgcgcgaataaataagatgatgttagtgaaaaaaatatctgaacatatttggaaaaatcatgcggctagacttgaacaccttaaacatgcggtacacacgatgaagcataaagatgggaaaaatagactcatgaactttatctatgagtactgctattaccatatgcaaggggaagaaatttttagcctcgcaagattttatgcaatccatcatgcaccaaagttgttcgacgttttttataattgttgtatcctagatacgatacgattcaaaagccttcttttagattgttcacatatcataggtaaaaacgctcatgatgctactaatatcaacatcgtgaacaagtatattgacaacctgtttgctatgggagttcttagcaaaaaagaaatcttacaggactatccatccatttattctaaacattatatgccttag

SEQ ID NO: 139 - DNA, 1596 bp, African swine fever virus
atgttttctctccagaacttgtgtcgaaaaacattacctgactgtaaacttcctgaattttttgacgaatatatattacaactgctgggattatactgggaaaaccatggaactattcaacgagcaggaaacaactgtgtgcttatacagcaacataccctcattcccgtaaacgaagccctgagaatagcagcatctgaagaaaattatgagatcgtgagccttttattagcgtgggagggaaacctttactatgctattataggagctctagagggcaaccgccacgacttaattcgtaaatatgatgaccaaatcaaggaccatcatgaaattctgccattcattgacgatccagtcatatttcacaaatgccatatcatgcggcgatgcttttttgattgtattttatatcaagctgtaaaatatagtaagtttcgcgttcttctttattttaaatataggttagagaatgatttgcccctcgctcatttacttattaaaaaggcatgtgaagatcataattatgaagttattaaatggatatatgaaaacctacatatctacaatataatggatacctttggatgtgctattgcccataaggatctacgtctatatcgtttggggtatacatttatatataacaggatcgtaccctataagtatcattatttagatgttctcattctttcaggcctacatctcctgtataaggtggcagccaaaggatacttagattttatcctagaaaccttaaagtatgatcataacaatgataatttagatattattctaacacaagctgcaacatataaccatagaaaaattttaacctattacattcctcaattaacctatgcacaaatagaacaatgtttgttcatggcgattaaaaaaaaatcttccaagaaaaccttgaacttactactgtctcacctaaagctttccatcaaactcatcaaaaaaataagccaatatgttgccacttacaattcaacaaatataataggcattctgaatatgcggcggaaaaagaagatatatttagatatcatattgacaaaatttgtaaaaaaagctatttttaataaatttgtcgttcgatgtatggatacattttccataaacccggaaagaatcatcaaaatggccgcgcgaataaataagatgttgttagtgaaaaaaatatctgaacatgcttggaaaaatcatgcggctagacttaaacaccttaagcatgcggtatacacgatgaaacataaagatgggaaaaatagactcatgaacttgatctatgatcactactattaccatatgcaaggggaagaaatctttagcctcgcaagattttatgcaatccatcatgcaccaaagttgtttgacgttttttatgattgttgtctcctagatactatacgatttaaaaaccttcttttagattgttcacacatcataggtaaaaacgctcatgatgctactaatatcactatcgtgaacaagtatattggcaacctgtttgctatgggagttcttagcaaaaaagaaatcttacaggactatccatccatctattctaaacattacatgccttag

SEQ ID NO: 140 - DNA, 1596 bp, African swine fever virus
atgttctctctccagaacttatgtcgaaaaacattacctgactgtaaacttcctgaattttttgacgattatatattacaactgctgggattatactgggaaaaccatggaactattcaacgggcaggaaacaactgtgtgcttatacaacaacataccctcattcccgtaaatgaagccctaagaatagcagcatctgaagaaaattatgagatcgtgggccttttattagcgtgggaggggaacctttactatgctattataggggctctagagggtaaccgctacaacttaattcgtaaatatgatgatcaaatcaaggaccatcatgacattctgccattcattgatgatccaatcatatttcacaaatgccatatcatgcggcgatgcttttttgattgtattttatatcaagctgtaaaatatagtaagtttcgtgttcttctttattttaaatatacattagaggatgatttgcccctcgttcatttacttattgaaaaggcatgtgaagatcataattatgaagttattaaatggatatatgaaaacctacatgtctgccatataatagatacctttgactgtgctattgcccataaagatctacgtttatattgtttggggtatacatttatatacaacaggattgtaccctataagtatcatcatttagatattctcatactttcaagcctacaactcctacataaggtggcggccaaaggatacttagattttatcctagaaaccttaaagtatgatcataatatagataatttagatgttattctaacacaagctgcaacatataaccatagaaaaattttaacctattttattcctcaatcaacctacgcacaaatagaacaatgtttgttcgtggcgataaaaacaaaatcttccaagaaaaccttgaacttactactgtctcacctaaacctttccatcaaactcatccaaaaaatcagccaatatgttgccactttcaattcaacaaatataataggcattctgagtatgaagcggaaaaagaagatatatttggatatcatattgacaaaatttgtaaaaaatgctatttttaataaatttgtcgttcgatgtatggagagattttctataaacccggaaagaatcgtcaaaatggctgcgcgtataaataagatgatgttagtgaaaaaaatatctgaacatgtttggaaaaatcatgcggctagacttaaacaccttaaacatgcggtacacacgatgaagcataaagatgggaaaaatagactcatgaactttatctatgaacactgctattaccatatgcaaggggaagaaatttttagcctcgcaagattttatgcaatccatcatgcaccaaagttgttcgacgttttttataattgttgtatcctagatacgatacgattcaaaagccttcttttagattgttcacatatcataggtaaaaacgctcatgatgctactaatatcaacatcgtgaacaagtatattggcaacctgtttgctatgggagttcttagcaaaaaagaaatcttacaggactatccatccatctattctaaacattatatgccttag

SEQ ID NO: 141 - DNA, 1596 bp, African swine fever virus; identical to SEQ ID NO: 140

SEQ ID NO: 142 - DNA, 1596 bp, African swine fever virus
atgttctctctccagaacttatgtcgaaaaacattacctgactgtaaacttcctgaattttttgacgaatatatattacaactgctgggattatactgggaaaaccatggaactattcaacgagcaggaaacaactgtgtgcttgtacaacaacataccctcattcccgtaaacgaagccctgagaatagcagcgtctgaagaaaattatgagatcgtgagccttttattagcgtgggagggaaacctttactatgctattataggagctctagagggcaaccgccacgacttaattcgtaaatatgatgaccaaatcaaggaccatcatgaaattctgccattcattgacaatccagtcatatttcacaaatgccatataatgcggcgatgcttttttgattgtattttatatcaagctgtaaaatatagtaagtttcgcgttcttctttattttaaatataggttagagaatgatttgcccctcgctcatttacttgttgaaaaggcatgtgaagatcataattatgaagttattaaatggttatatgaaaacctacatatctacaatataatggagacctttgaatgtgctattgcccataaggatctacgtctatatcgtttggggtatacatttatatataacaggatcgtaccctataagtatcattatttagatgttctcattctttcaggcctacatctcctgtataaggtggcagccaaaggatacttagattttatcctagaaaccttaaagtatgatcataacaatgataatttagatattattctaacacaagctgtaacatataaccatagaaaaattttaacctattacattcctcaattaacctatgcacaaatagaacaatgtttgttcatggcgattaaaaaaaaatcttccaagaaaaccttgaacttactactgtctcacctaaagctttccatcaaactcatcaaaaaaataagccaatatgttgccacttacaattcaacaaatataataggcattctgaatatgaagcggaaaaagaagatatatttagatatcatattgacaaaatttgtaaaatacgctatttttaataaatatgtcgttcgttgtatggatacattttccataaacccggaaagaatcatcaaaatggccgcgcgaataaataagatgttgttagtgaaaaaaatatctcaacatgcttggaaaaatcatgcggctagacttaaacaccttaagcatgcggtatacacgatgaaacataaagatgggaaaaatagactcatgaacttgatctatgatcactactattaccatatgcaaggggaagaaatctttagcctcgcaagattttatgcaatccatcatgcaccaaagttgtttgacgttttttatgattgttgtctcctagatactatacgatttaaaagccttcttttagattgttcacacatcataggtaaaaacgctcatgatgctactaatatcactatcgtgaacaagtatattggcaacctgtttgctatgggagttcttagcaaaaaagaaatcttacaggactatccatccatctattctaaacattacatgccttag

SEQ ID NO: 143 - DNA, 1593 bp, African swine fever virus
atgttttctctccagaacttatgtcgaaaaaccttacctgaccgtaaacttcctgaattttttgacgattatgtattacaactgctgggattatactgggaaaaccatggaacgattcaacgagcaggaaacaactgtgtacttatccaacaacataacctcattcccgtaaatgaagccctaagaatagcagcatctgaagaaaattatgagatcgtgagccttttattagcttgggaggggaacctttactatgctattataggggctctagagggtaatcgccacaacttaattcgtaaatatgatgaccaaatcaaggaccatcatgaaattctgccatttattgacgatccagtcatatttcacaaatgtcatatgatgcggcgatgcttttttaattgtattttgtatcaagctgtaaaatatagtaagtttagcgttcttctatattttaaatatatattaaaggagaatttgcccctcgttcattcacttattgaaaaggcgtgtgaagatcataattatgaagttattaaatggatctatgaaaacctacatatctatgaaataatggatacctttaaatgtgctattgcccataaagatctacatttatatagtttaggatatacatttatatataataggatcgtaccctataagtatcatcatttagatattcgcatactttcaaggctacaacttctacataaggtgacagccaaaggatacttggactttatcctagaaaccttaaagtatgatcataataaagataatataaatattattctgacacaagctgcaacctataaccatagaaacattttaacctatttcattcctcaatcaacctacgcgcagatagaacaatgcttattcgtggcaataaaaacaaacgcttccaagaaaaccttgaatttactattatctcacctaaacctttctatcaaactcgtcaaaaaattaagccaatatgttgttgcttacaagtcaacaaatatcataagcattctgagtatgcagcaaaaaaagaagatatatttagatatcattttgacaaaggttgtaaaaaacgctatttttattaaatttgtcattggatgtatggttacattttccataaacccagaaagaattgtcaaaatggccgcgcgaataaaaaagatgaagttagtgaaaaatatatctgaacatgtttggaaaaatcatgcggctaaacttaaacaccttaagcatgcggtacacacgatgaagcatcaagaaggaaaaaatagattaatgaactttatctatgatcactgttattaccacatgcaaggagaagagatctttagtcttgcaagattttatgcaatccatcatgcgccaaaattgttcgacgttttttatgattgttgtatcctagatacgatacgatttaaaagcctgcttttagattgttcatatatcatagctaaaaacgctcatgatgctagtattaacatcgtgaataagtatattggcaatctatttgctatgggggttcttagcaaaaaagaaatcttacaagactatccatccatctattctaaggattacatgctttag

SEQ ID NO: 144 - DNA, 1593 bp, African swine fever virus
atgttctctctccagaacttatgtcgaaaaaccttacctgactgtaaacttcctgagttttttgacgattatgtattacaactgctgggcttatactgggaaaatcatggtacgattcaacgagcaggaaacaactgtgtacttatccaacaacataacctcattcccgtaaatgaagccctaagaatagcagcatctgaggaaaattatgagatcgtgagccttttattagcgtgggaggggaacctttactatgctattataggggctctagaggggaaccaccacaacttaattcgtaaatacgatgaccaaatcaaggaccatcatgaaattctgccatttattgacgatccagtcatatttcacaaatgtcatacaatgcggcgatgcttttttaattgtattttatatcaagctgtaaaatatagtaagttcagcgttcttctatactttaaatatatattaaaggagaatttgcccctcgtccactcacttattgaaaaggcgtataaatatcataattatgaggttattaaatggatctatgaaaacctacatatctatgatatcataaatacttttaaatgtgctattgcccataaagatctacgtttatattgtttagggtatacatttgtatataatagaatcgtaccctataagtattatcatttagatattcgcatccttttaaggctacaacttctacataaggtgacagccaaaggatacttggattttatcctagaaaccttaaagtatgatcataatacaaacaatatagatattatcctaacacaggctgcaacctataaccatagaaacattttaacctatttcattcctcaatcaacctacgcacaaatagaacaatgtttattcgtggcgataaaaacaaatgcttccaagaaaaccttgaatttactattatctcacctaaatctttctatcaaactcgtcaaaaaattaagccaatatgttgtcgcttacaagtcaacaaatatcataagcattctgagtaggcagcaaaaaaagaagatatatttagatatcattttgacaaaggttgtaaaaaatgctgtttttaataaatttgtcattggatgtatggttacattttccataaacccagaaagaattgtcaaaatggccgcgcgaataaaaaagatgaagttagtaaaaaatatatctgaacatgtttggaaaaatcatgcggttaaacttaaatatcttaagcatgcggtacacacgatgaagcatcaagaaggaaaaaatagattaatgaactttatctatgatcactgttattaccacatgcaagaagaagagatctttagcctcgcaagattttatgcaatccatcatgcgccaaaattgttcgacgttttttatgattgttgtatcctagatacgatacgatttaaaagcctgcttttagattgttcacatatcatagttaaaaacgctcatgatgctagtattaacatcgtgaacaagtatattggcaatctatttgctatgggagttcttagcaaaaaagaaatcttacaagactatccatctatctattctaaggattacatgctttag

SEQ ID NO: 145 - DNA, 1593 bp, African swine fever virus
atgttctctctccaaaacttatgtcgaaaaaccttacctgactgtaaacttcctgaattttttgacgattatgtattacaactgctgggattatactgggaaaaccatggaacgattcaacgagcaggaaacaactgtgtacttatccaacaacataacctcattcccgtaaatgaagccctaagaatagcagcatctgaagaaaattatgagatcgtgagccttttattagcgtgggaggggaacctttactatgctattataggggctctagagggtaaccgccacaacttaattcgtaaatatgatgaccaaatcaaggaccatcatgaaattctgccatttattgacgatccagttatatttcacaaatgtcatataatgcggcgatgcttttttaattgtatgttgtatcaagctgtaaaatatagtaagtttagcgttcttctatattttaaatatatattaaaggaaaatttgcccctcgtccactcactcattgaaaaagcgtataaatatcataattatgaggttattaaatggatctatgaaaatctacatatctatgatatcataaatacttttaaatatgctattgcccataaagatctacgtttatattgtttagggtatacatttgtatataataggatcgtaccctataagtattatcatttagatattcgcatccttttaaggctacaacttctacataaggtgacagccaaaggatacttggattttatcctagaaaccttaaagtatgatcataatacaaacaatatagatattatcctaacacaggctgcaacctataaccatagaaacattttaacctatttcattcctcaatcaacctacgcacaaatagaacaatgcttattcgtggcaataaaaacaaacgcttccaaaaaaaccttgaatttactattatctcacctaaacctttctatcaaactcgtcaaaaaattaagccaatatgttgtcgcttacaagtcaacaaatatcataagcattctgagtatgcagcaaaaaaagaagatatatttagatatcattttgacaaaggttgtaaaaaacgctgtttttattaaatttgtcattggatgtatggttacattttccataaacccagaaagaattgtcaaaatggccgcgcgaataaaaaagatgaagttagtaaaaaatatatctgaacatgtttggaaaaatcatgcggctaaacttaaacatcttaagcatgcggtacacacgatgaagcatcaagaaggaaaaaatagactcatgaactttatctatgatcactgctattaccacatgcaaggagaagagatctttagtcttgcaagattttatgcaatccatcatgcgccaaaattgttcgacgttttttatgattgttgtatcctagatacgatacgatttaaaagcctgcttttagattgttcacatattatagctaaaaacgctcatgatgctagtattaacatcgtgaataagtatattggcaatctatttgctatgggagttcttagcaaaaaagaaatcttacaagactatccatccatctattctaaatatgacatactttag

SEQ ID NO: 146 - DNA, 1593 bp, African swine fever virus
atgttctctctccagaacttatgtcgaaaaacattacctgactgtaaacttcctgaattttttgacgaatatatattacaactgctgggattatactgggaaaaccatggaactattcaacgagcaggaaacaactgtgtgcttatccagcaacataaccttattcccgtaaatgaagccctaagaatagcagcatctgaagaaaattatgagatcgtgagccttttattagcgtgggagggaaacctttactatgctattataggggctctagagggcaaccgccccgacttaattcgtaaatatgatgaccaaatcaaggaccatcatgaaattttgccattcatcgacgatccaatcatatttcacaaatgtcacataatgcggcgatgcttttttaattgtattttatatcaagctgtaaaatatagtaagtttcgtgttcttctttattttaaacataggttaggggatgatttgcccctcactcatttacttattgaaaaggcatgtgaagatcataattatgaagttattaaatggatctatgaaaacctacatagctacaatataatggatacctttgaatgtgctattgcccataaggatctacgtctatattgtttggggtatacatttatatataataggatcgtaccctataagtatcatcatttagatatttgcatactttcaagcctacaactcctgcataaggtggcagccaaaggatacttggattttatcctagaaaccttaaagtatgatcataacataaataatatagatattattctaacacaagctgcaacctataaccatagaaaaattttaacctatttcattcctcaattaacctacgcacagatagaacaatgtttactagtggcgataaaaacaaaagcttccaagaaaaccttgaacttactactgtctcacctaaacctttccatcaaactcatcaaaaaaataagccaatatgttgtcacttacaattcaacaaatatcataagcattctgagtatgcggcggaaaaagaagatatatttagatatcattttgacagagtttgtaaaaaacgctatttttaataaatttgtcgttcgatgtatggatacattttccataaacccggaaagaattgtcaaaatggccgcacgaataaataggatgatgttagtgaaaaatatatctgaacgtgtttggaaaaatcatgcggttaaacttaaacaccttaagcatgcggtacatacgatgaagcatcaagaaggaaaaaatagactcatgaactttatctatgatcactgctattaccacatgcaaggggaagagatctttggcctcgcaagattttatgcaatccatcatgcacccaagttgtttgacgttttttatgattgctgcatgctagatgctacacgatttaaaagcctgcttttagattgtccacatatcataggtaaaaacgcttatgatgctggtatcaacctcgtgaacaaatatattggcaacctatttgctatgggggttcttagcaaaaaagaaatcttacaggactatccatctatctattctaaacatgatatgttttag

SEQ ID NO: 147 - DNA, 1581 bp, African swine fever virus
atgttttcccttcaagacctttgccgaaagcatctttttattcttcccgatgtttttggcgagcatgtactacaacgattaggactgtattggagatgtcacggctcccttcaacgcataggagacgaccacatactcatacgacgggatctcatcctttccaccaacgaggccttaagaatggcgggagaggaaggaaacaatgaagtagtaaagctcttgttactgtggaagggaaatcttcattacgccgtcataggagccttgcagggtgatcaatatgacctgatccataagtatgaaaaccaaatcggcgactttcattttatcttaccattgattcaagacgcgaatacgtttgaaaaatgccacgctttagaacgtttttgtggtgtttcatgtctgctaaaacatgctacaaaatacaacatgctccctattctccaaaaataccaagaagagctgtctatgagagcgtatcttcacgaaaccctatttgaactagcatgcctatggcagaggtatgatgtccttaaatggatagagcaaaccatacatgtttacgacctaaagattatgtttaatattgccatctccaagagggatctgactatgtactccttaggatatattttcctttttgatagagggaacaccgaagctacgttgctaacgcaacatctcaagaagacagcggccaaagggctcctccactttgtgctagaaacgttaaaatacggcggcaacatagataccgtcctgacccaagccgtaaagtacaatcatagaaaacttttagattattttctgcgtcaactacctcgtaaacatattgaaaaacttttgttgctggccgtgcaggaaaaggcttctaaaaaaacattgaacttactgttgtcacatttaaactactccgtgaaacgcatcaaaaaactaccgcgctatgtgatagagtacgagtccaccttggtgataaagattttattaaaaaaaagagtgaacctgatagatgccatgttggaaaagatggtaagatatttttctgcgacgaaagtgaggacgatcatggatgagctttcgattagtccggaaagagtcattaagatggctatacagaaaatgagaacggatatcgtaatccatacttcttatgtttgggaggatgatctagaacgtcttactcgtcttaaaaatatggtatacaccataaagtacgaacatgggaaaaaaatgttaattaaagtcatgcacggcatatacaaaaacttattatacggcgaaagggaaaaagtcatgttttatttagccaagctctatgttgctcaaaacgcggccacccaattcagagacatttgtaaggactgttacaaactggatgtggcacggtttaaaccgcggtttaagcaactaatattagactgtttagaaattattactaaaaaatcttgctatagtatcctggaaatcttagaaaaacatattatttccctgtttactatgaaagttatgactgaagaagaaaaaaacctatgtttagaaatattatataaagtaattcattataaaacaatacaatgttaa

SEQ ID NO: 148 - DNA, 1581 bp, African swine fever virus; identical to SEQ ID NO: 147

SEQ ID NO: 149 - DNA, 1581 bp, African swine fever virus
atgttttcccttcaagacctttgccgaaagcatctttttattcttcccgatgtttttggcgagcatgtactacaacgattaggactgtattggagatgtcacggctcccttcaacgcataggagacgaccacatactcatacgacgggatctcatcctttccaccaacgaggccttaagaatggcgggagaggaaggaaacaatgaagtagtaaagctcttgttactgtggaagggaaatcttcattacgccgtcataggagccttgcagggtgatcaatatgacctgatccataagtatgaaaaccaaatcggcgactttcattttatcttaccattgattcaagacgcgaatacgtttgaaaaatgccacgctttagaacgtttttgtggtgtttcatgtctgctaaaacatgctacaaaatacaacatgctccctattctccaaaaataccaagaagagctgtctatgagagcgtatcttcacgaaaccctatttgaactagcatgcctatggcagaggtatgatgtccttaaatggatagagcagaccatgcatgtttacgacctaaagattatgtttaatattgccatctccaagagggatctgactatgtactccttaggatatattttcctttttgatagagggaacaccgaagctacgttgctaacgcaacatctcgagaagacagcggccaaagggctcctccactttgtgctagaaacgttaaaatacggcggcaacatagataccgtcctgacccaagccgtaaagtacaatcatagaaaacttttagattattttctgcgtcaactacctcgtaaacatattgaaaaacttttgttgctggctgttcaggaaaaggcttctaaaaaaacattgaacttactgttgtcacatttaaactactccgtgaaacgcatcaaaaaactgctgcgctatgtgatagagtatgagtccaccttggtgataaagattttgttaaaaaaaagagtaaacctgatagatgccatgttggaaaagatggtaagatatttttctgcgacaaaagtgaggacgatcatggatgagctttcgattagtccggaaagagtcattaagatggctatacagaaaatgagaacggatatcgtaatccatacttcttatgtttgggaggatgatctagaacgttttactcgtcttaaaaatatggtatacaccataaagtacgaacatgggaaaaaaatgttaattaaagtcatgcacggcatatacaaaaacttattatacggcgaaagggaaaaagtcatgtttcatttagccaagctctatgttgctcaaaatgcggccacccaattcagagacatttgtaaggactgttacaaactggatgtggcacggtttaaaccgcggtttaagcaactaatattagactgtttagaaattgttactaaaaaatcttgctatagtatcctggaaatcttagaaaaacatattatttccctatttactatgaaagttatgactgaagaagaaaaaaacctatgtttagaaatattatataaagtaattcattataaaacaatacaatgttaa

SEQ ID NO: 150 - DNA, 1581 bp, African swine fever virus
atgttttcccttcaagacctttgccgaaagcatctttttattcttcccgatgtttttggcgagcatgtactacaacgattaggactgtattggagatgtcacggctcccttcaacgcataggagacgaccacatactcatacgacgggatctcatcctttccaccaacgaggccttaagaatggcgggagaggaaggaaacaatgaagtagtaaagctcttgttactgtggaagggaaatcttcattacgccgtcataggagccctgcagggtgatcaatatgacctgatccataagtatgaaaaccaaatcggcgactttcattttatcttaccattgattcaagacgcgaatacgtttgaaaaatgccacgctttagaacgtttttgtggtgtttcatgtctgctaaaacatgctacaaaatacaacatgctccctattctccaaaaataccaagaagagctgtctatgagagcgtatcttcacgaaaccctatttgaactagcatgcctatggcagaggtatgatgtccttaaatggatagagcagaccatgcatgtttacgacctaaagattatgtttaatattgccatctccaagagggatctgactatgtactccttaggatatattttcctttttgatagagggaacaccgaagctacgttgctaacgcaacatctcgagaagacagcggccaaagggctcctccactttgtgctagaaacgttaaaatacggcggcaacatagataccgtcctgacccaagccgtaaagtacaaccatagaaaacttttagattattttctgcgtcaactacctcgtaaacatattgaaaagcttttgttgctggctgttcaggaaaaggcttctaaaaaaacattgaacttactgttgtcacatttaaactactccgtgaaacgcatcaaaaaactgctgcgctatgtgatagagtatgagtccaccttggtgataaagattttgttaaaaaaaagagtaaacctgatagatgccatgttggaaaagatggtaagatatttttctgcgacgaaagtgaggacgatcatggatgagctttcgattagtccggaaagagtcattaagatggctatacagaaaatgagaacggatatcgtaatccatacttcttatgtttgggaggatgatctagaacgtcttactcgtcttaaaaatatggtatacaccataaagtacgaacatgggaaaaaaatgttaattaaagtcatgcacggcatatacaaaaacttattatacggcgaaagggaaaaagtcatgtttcatttagccaagctctatgttgctcaaaatgcggccacccaattcagagacatttgtaaggactgttacaaactggatgtggcacggtttaaaccgcggtttaagcaactaatattagactgtttagaaattgttactaaaaaatcttgctatagtatcctggaaatcttagaaaaacatattatttccctatttactatgaaagttatgactgaagaagaaaaaaacctatgtttagaaatattatataaagtaattcattataaaacaatacaatgttaa

SEQ ID NO: 151 - DNA, 1581 bp, African swine fever virus; identical to SEQ ID NO: 150

SEQ ID NO: 152 - DNA, 1554 bp, African swine fever virus
atgttttcccttcaagacctttgccgaaagcatctttttattcttcccgatgtttttggcgagcatgtactacaacgattaggactgtattggagatgtcacggctcccttcaacgcgtaggagacgaccacatactcatacggcgggatctcatcctttccaccaacgaggccttaagaatggcgggagaggaaggaaacaatgaagtagtaaagctcttgttactgtggaagggaaatcttcattacgccgtcataggagccttgcagggtgatcaatatgacctgatccataagtatgaaaaccaaatcggcgactttcatcttatcttaccattgattcaagacgcgaaaacgtttgaaaaatgccatgctttagaacgtttttgtggtgtttcatgtctgctaaaacatgctacaaaatacaacatgctccctgttctccaaaaataccaagaagagctgtccatgagagcatatctttgcgaaaccctatttgaactagcatgcctatggcagaggtatgatgtccttaaatggatagagcaaaccatgcatgtttacgacctaaagattatgtttaatattgccatctccaagagggatctgactatgtactccttaggatatattttcctttttgatagagggaacaccgaagctacgttgctaacgcaatatctcgagaagacagcggccaaagggctcctccactttgtgctagaaacgttaaaatacggcggtaacatagatattgtcctgacccaagccgtaaagtacaatcatagaaaacttttagattattttctgcgtcaactacctcgtaaacatattgaaaagcttttgttgctggccgtgcaggaaaaggcttctaaaaaaacattgaacttactgttgtcacatttaaactactccgtgaaacgcatcaaaaaactgctgcgctatgtgatagagtacgagtccaccttggtgataaagattttattaaaaaaaagagtaaacctgatagatgccatgttggaaaagatggtaagatatttttctgcgacgaaagtgaggacgatcatggatgagcttttgattagtccggaaagagtcattaagatggctatacagaaaatgagaacggatatcgtaatccatacttcttatgtatgggaggatgatctagaacgacttactcgtcttaaagatatggtatacaccgtaaagtacgaacatgggaaaaaaatgttaattaaagtcatacacggcatatacaaaaacttattatacggcgaaaaagaaaaggtcatgtttcatttagccaagctctatgttgctcaaaacgcggccacccaattcagagacatttgtaaggactgttacaaactggatgtggcacggtttaaaccgcggtttaagcaactaatgttagactgtttagaaattgttactaaaaaatcttgctatagtattctggaaatcttagaaaaacatatgatttccctatttactatgaaagttatgactgaagaagaaaaaaacctatgtttagaaatattatataagtaa

SEQ ID NO: 153 - DNA, 1554 bp, African swine fever virus
atgttttcccttcaagacctttgccgaaagcatctttttattcttcccgatgtttttggcgagcatgtactacaacgattaggactgtattggagatgtcacggctcccttcaacgcgtaggagacgaccacatcctcatacggcgggatctcatcctttccaccaacgaggccttaagaatggcgggagaagaaggaaacaatgaagtagtaaagctcttgttactgtggaagggaaatcttcattacgccatcataggagccttgcagggtgatcaatatgacctgatccataagtatgaaaaccaaatcggcgactttcatcttatcttaccattgattcaagatgcgaaaacgtttgaaaaatgccacgctttagaacgtttttgtggtgtttcatgtctgctagaacatgctacaaaatacaacatgctccctattctccaaacataccaagaagagctgtctatgagagcatatcttcgcgaaaccctatttgaactagcgtgcctgtggcagaggtatgatgtccttaaatggatagagcaaaccatgcatgtttacgacctaaagattatgtttaatattgccatctccaagagggatctgacgatgtattccttaggatatattcttctttttgatagagagaacaccgaagctacgttgttaacacaacatctcgagaagacagcggccaaagggctcctctactttgtgctagaaacgttaaaatacggcggtaacatagatatcgtcctgactcaagccgtaaagtacaatcatagaaaacttttagattattttctgcgtcaactacctcgtaaacatattgaaaagcttttgttgctggccgtgcaggaaaaggcttctaaaaaaacattgaacttactgttgtcccatctaaactactccgtgaaacgtatcaaaaaactgctgcgctatgtgatagagtacgagtccaccttggtgataaagattttattaaaaaaaagagtaaacctgatagatgccatgttggaaaagatggtaagatatttttctgcgacgaaagtgaggacgatcatggatgagcttttgattagtccggaaagagtcattaagatggctatacagaaaatgagaacggatatcgtaatccatacttcttatgtttgggaggatgatctagaacgacttactcgtcttaaaaatatggtatacaccataaagtacgaacatgggaaaaaaatgttaattaaagtcatgcacggcatatacaaaaacttattatacgacgaaagagaaaaggtcatgtttcatttagccaagctctatgttgctcaaaacgcggccacccaattcagagacatttgtaaggactgttacaaactggatgtggcacggtttaaaccgcggtttaagcaactaatgttagactgtttagaaattgttactaaaaaatcttgctatagtatcctggaaatcttagaaaaacatattatttccctatttactatgaaagttatgactgaagaggaaaaaaacctatgtttagaaatattatataagtaa

SEQ ID NO: 154 - DNA, 1569 bp, African swine fever virus
atgttttcccttcaagacctttgccgaaagcatctttttattcttcccgatgtttttggcgagcatgtattacaacgattaggactgtattggagatgtcacggctcccttcaacgcgtaggagacgaccacatactcatacggcgggatctcatcctttccaccaacgaggccttaagaatggcgggagaagaaggaaacaatgaagtagtaaagctcttgttactgtggaagggaaatcttcattacgccatcataggagccttgcagggtgatcaatatgacctaatccataagtatgaaaaccaaatcggcgactttcatcttatcttaccattgattcaagatgcgaaaacgtttgaaaaatgccacgctttagaacgtttttgtggtgtttcatgtctgctaaaacatgctacaaaatacaacatgctccctattctccaaacataccaagaagagctgtctatgagagtatatcttcgcgaaaccctatttgaactagcatgcctatggcagaggtatgatgtccttaaatggatagagcaaaccatgcatgtttacgacctaaaggttatgtttaatattgccatctccaagagagatctgactatgtattccttaggatatattcttctttttgatagagagaacaccgaagctacgttgctaacgcaacatctcgagaaaacagcggccaaggggctcctccactttgtgctagaaacgttaaaatacggcggtaacatagatatcgtcctgtcccaagccgtaaagtacaatcatagaaaacttttagattattttctgcgtcaactacctcgtaaaaatattgaaaaacttttgttgctggctgttcaggaaaaggcttctaaaaaaacattgaacttactgttgtcccatttaaactactccgtgaaacgcatcaaaaaactgctgcgctatgtgatagagtacgagtccaccttggtgataaagattttattaaaaaaaagagtaaacctgatagatgccatgttggaaaagatggtaagatatttttctgcgacgaaagtgaggacgatcatggatgagctttcgattagtccggaaagagtcattaagatggctatacagaaaatgagaacagatatcgtaatccatacttcttatgtttgggaggatgatctagaacgacttactcgtcttaaaaatatggtatacgccataaagtatgaacatgggaaaaaaatgttaatgaaagtcatgcacggcatatacaaaaacttattatacggcgaaagagaaaaggtcatgtttcatttagccaagctctatgttgctcaaaacgcggccacccaattcagagacatttgtaaggactgttacaaactggatgtggcacggtttaagcaactaacgttaaactgtttagaaattattactaaaaaatcttgctatagtatcctggaaatcctagaaaaacatattatttccctatttactatgaaagttatgactgaagaagaaaaaaacctatgtttagaaatattatataaagtaattcattataatacaatacaatgttaa

SEQ ID NO: 155 - DNA, 1581 bp, African swine fever virus
atgttttcccttcaagatctttgccgaaagcatctttttattcttcccgatgtttttggcgagcatgtactacaacagttaggactgtactggaaacgtcacggctctcttcaacgcataggagacgaccacatactcatacggcgggacctcatcctttccaccaacgaggccttaaaaatggcgggagaggaaggaaacaatgaagtagtaaagctcttgttactgtgggagggaaaccttcattatgccatcataggggccttacagggtgatcaatatgatctgatccataagtatgaaaaccaaatcgaagactatcatcatatcttgccattgattcaagatgcgaaaacgtttgaaaaatgccacgccttagaacgtttttgtgatgttccatgtctgctagaacatgctacaaaacacaacatgctccctattctccaaaaataccaagaagagctgtctataagagtgtatcttcgcgaaaccctattcgaactagcatgcctatggcagaggtatgatgttcttaaatggatagagcaaaccatgcatgtttacgacctaaagattatgtttaatattgccatctccaagagggatctaagcatgtactccttaggatatgttctcctttttgatagaggaaacatcgaagctacgttcctaacgcaacatctcgagaagacagcggccaaagggctcctccactttgtgctagaaacgttaaaatacggcggtaacttaaatatcgtcctgtcccaagccgtaaaatacaatcatagaaaacttttggattattttctgcgtcaactacctcgtaaaaatattgaaaaacttttgttgctggccgtgcaggaaaaggcttctaaaaaaacattgaacctactgttgtcacacttaaactactccgtgaaacacatcaaaaaactgctgcgctatgtgatagagtacgagtctaccttggtgataaaacttttattgaaaaaaagagtaaacctgatagacgccgtgttggaaaagaatgtaagatatttttctgcgattaaagtgaggactattatggatgagctttcgattagtccggaaagagtcattaagatggccatacagaaaatgagaacggatattgtgattcagacttcttatatttgggaggatgatctagaaagacttattcgtcttaaaaatatggtatacaccataaagtatgaacatgggaaaaaaatgttaattaaagttattcacggcatatacaaaaacttattatacggcgaaaaagaaaaagtcatgtttcatttagccaaactctatgttgctcaaaacgcggccacccaattcagggacatttgtaaggactgttgcaaactggatgtggcgcggtttaaaccgcggtttaaacaactaattttagactgtttagaaatggttactaaaaaatcttgctttagtattatagaaatcttagaaaactatattatttccctatttgtgatgaaagtcatcactgaagaagaaaaaaacctatgtttagaactattatataaagtaattagttacaaaacgatataatgttaa

SEQ ID NO: 156 - DNA, 1581 bp, African swine fever virus
atgttttcccttcaagaccattgccgaaagcatctttttattcttcccgatgtttttggcgagcatgtactacaacggttaggactgtactggggacgtcacggctctcttcaacgaatcggggacgatcacatactcatacggcgggacctcatcctttccaccaacgaggccttaaaaatggcgggagaagaaggaaacaatgaagtagtaaagctcttgttactgtgggagggaaatcttcattatgctatcataggggctttacagggtgatcaatatgacctaatccataagtatgaaaaccaaatcgaagactatcatcatatcttaccattgattcaagatgcggaaacgtttgaaaaatgccacgccttagaacgtttttgtgatgttccatgtctgctagaacatgctacaaaacacaacatgctccctattctccaaaaatatcaagaagagttgtctataagagtgtatctacgcgaaaccctattcgaactagcatgcctatggcagaggtatgatattcttaaatggatagagcaaaccatgcatgtttacgatctaaaaattatatttaatattgccatctccaagagggatctaagcatgtactccttaggatatgttctcctttttgatagagggaacaccgaagctaccttgttaacgcaacacctcgagaagacagcggccaaggggctcctccactttgtgctagaaacgttaaaatacggcggtaacttaaatatcgtcctgttccaagccgtaaaatacaatcatagaaaacttttagattattttctgcgtcaactaccccgtaaaaatattgaaaaacttttgttgctggctgtgcaggaaaaagcttctaagaaaacattgaacttactgttgtcacatttaaactactccgtgaaacgcatcaaaaaactgctgcgctatgtgatagagtacgagtctaccttggtgataaagattttattaaaaaaaagagtaaacctaatagacgccgtgttggaaaagactgtaagatatttttctgagacgaaagtaaagactattatggatgagctttcgattaatccggaaaaagtcattaagatggccatacagaaaatgagaacggatattgtgatccaaacttcttatatttgggaggatgatctagaaagacttattcgtcttaaaaatatggtatacaccataaagtatgaacatgggaaaaaaatgttaatgaaagttattcacggcatatacaaaaacttattacacgatgaaaaagaaaaagtcatgtttcatttagccaagttctatattgctcaaaacgcggccacccaattcagagacatttgtaaagactgttgcaaactggatgtggcgcggtttaaaccgcggtttaaacaactaattttagactgtttagaaattgttactaaaaaatcttgctttagtattatagaaattttagaaaaccatattatttccctatttatgatgaaagttatcactgaagatgaaaaaaacctaggtttagaattattatataaagtaattagttacaaaatgatatcatattaa

SEQ ID NO: 157 - DNA, 1581 bp, African swine fever virus
atgttttcccttcaagacctttgccgaaagcatctttttattcttcccgatgtttttggcgagcacgtactacaacaattggggctgtactgggaaaaacacggctctcttcaacgaatagggaacgatcacatactcatacggcgggatctcatcctttctatcaacgaggccttaaaaatagcggcagaggaaggaaacaatgaagtagtaaagcttttgttactatggaagggaaatcttcattatgccatcataggagctttggagggtgaccaatatgacctgatctatacgtatgaaaaccaaattgaagactatcatcatatcttgccattgattcaagatgcgaaaacgtttgaaaaatgccacgccttggaacgtttttgtgatgttccatgcctgctagagcatgctataaaacacaacatgctccctattctccaaaaatatcaagaagaactgtttataagagtgtatctccgcgaaaccctatttgaactagcatgcctatggcagaggtatgatattcttaaatggatagagcaaaccatgcatgtttacgatctaaaaattatatttaatattgccatctccaagagggatctaagcatgtactccttaggatatgttctcctttttgatagagggaacaccgaagctaccttgttaacgcaacacctcgagaagacagcggccaaggggctcctccactttgtgctagaaacgttaaaatacggcggtaacataaacatcgtcctatcccaagccgtaaaatacaatcataggaaacttttagattattttctgcgtcaactacctcgtaaaaatattgaaaaacttttgttgttagccgtacaggaaaaggcttctaagaaaacattgaacttactgttgtcttatttaaactactccgtgaaacgcatcaaaaaactgctgcgctatgtgatagagtacgagtccacactggtgataaggattttattaaaaaaaagaataaacctaatagacgccgtgttggaaaagactgtaagatatttttctgagacgaaagtaaagactattatggatgagctttcgattaatccggaaaaagttattaagatggccatacagaaaatgagaacggatattgtgatccaaacttcttatatttgggaggatgatctagaaagacttattcgtcttaaaaatatggtatacaccataaagtatgaacatgggaaaaaaatgttaatgaaagttattcacggcatatacaaaaacttattacacgatgaaaaagaaaaagtcatgtttcatttagccaagttctatattgctcaaaacgcggccacccaattcagagacatttgtaaagactgttgcaaactggatgtggcgcggtttaaaccgcggtttaaacaactaattttagactgtttagatattattactaaaaaaacgtgcctcaatattatggaaatcttagaaaatcatattatttcgctatttgctatgaaaatgatgactgaagatgaaaaaaacctaggtttagaattattatataaagtaattagttacaaaatgatatcatattaa

SEQ ID NO: 158 - DNA, 1575 bp, African swine fever virus
atgttttcccttcaagacctttgccgaaagcatctttttattcttcccgatgtttttggcgagcatgtactacaacaattggggctgtactgggaaaaacacggctctcttcaacgaatagggaacgatcacatactcatacggcgggatctcatcctttctatcaacgaggccttaaaaatagcggcagaggaaggaaacaatgaagtagtaaagctcttgttactgtggaagggaaatcttcattatgccatcataggagccttgcagggtgaccaatatgacctcatccatacgtacgaaaaccaaatcgaagactatcatcatatcttgccattgattcaagatgcgaaaacgtttgaaaaatgccacgccttggaacgtttttgtgatgttccatgcctactagaacatgctacaaaacacaacatgctccctattctccaaaaataccaagaagagctgtttataagagtgtatctccgcgaaaccctatttgaactagcatgcctatggcagaggtatgatatccttaaatggatagagcaaaccatgcatgtttacgacctaaagattatgtttaatattgccatctccaagagggatctaagcatgtactctttagaatatattctcttttttaatagagggaacaccgatgttgcgttagcaacgttgctaacgcaacatctcgagaagacagcggccaaggggctcctccactttgtgctagaaacgttaaaatacggcggtaacataaacatcgtcctctcccaagccgtaaaatacaatcatagaaaacttttagattattttctgcgtcaactacctcgtaaaaatattgaaaaacttttgttgctggccgtacaggaaagggcttctaagaaaacattgaacttactgttgtcttatttaaactactccgtgaaacgcatcaaaaaactgctgcgctatgtgatagagcatgagtccacactggtgataaggattttattaaaaaaaagaataaacctaatagacgccgtgttggaaaagactgtaagatatttttctatgactaaagtgaggacgatcatggatgagctttcgattaatccggaaaaagtcattaaaatggccgtgcagaaaatgagaacggatatcgtgatccatacttcttatgtttgggaggatgatctagaaagacttattcgtcttaaaaatatgctatacactataaagtatgagcatggaaaaaaaatgctaattaaagtcattcacggcatatacaaaaacttatacggcgaaaaagaaaaagtcatgtttaatttggccaagttctatgctgctcaaaacgcggccacccaattcaaagacacttgtaaagactgttgcaaactggatgtggcgcggtttaaacaactaattttagactgtttagacattattactaaaaaaacgtgcctcagtatcatggaaatcttagaaaatcatattatttcgctatttgctatgaaaatgatgacggaggatgaaaaaaacttaggtttagaaatattatataaagtaattagttacaaaatgatatcatattaa

SEQ ID NO: 159 - protein, 350 aa, African swine fever virus
MLPSLQSLTKKVLAGQCVPTNQHYLLKCYDLWWHDAPITFDHNLRLIKSAGIKEGLNLNTALVKAVRENNYNLIKLFAEWGADINYGLVSVNTEHTWDLCRELGAKETLNEEEILQIFIDLKFHKTSSNIILCHEVFSNNPILQKVNNIKMRIEIFWELRELIVKTDLLNNEFSLSTLLLKYWYAIAIRYNLKEAIQYFYQKYTHLNTWRLTCALCFNNVFDLHEAYEKDKIHMDIEEMMRIACIKDHNLSTMYYCYVLGANINQAMLSSIQYYNIENMFFCIDLGADVFEEGTTALGEGYELIKNILSLKIYSPATTPLPKSTDPEIIDHALKNYVSKNMMIFLTYDLR

SEQ ID NO: 160 - protein, 350 aa, African swine fever virus; identical to SEQ ID NO: 159

SEQ ID NO: 161 - protein, 350 aa, African swine fever virus
MLPSLQSLTKKVLAGQCVPTNQHYLLKCYDLWWHDAPITFDHNLRLIKSAGIKEGLNLNTALVKAVRENNYNLIKLFAEWGADINYGLVSVNTEHTWDLCRELGAKETLNEEEILQIFIDLKFHKTSSNIILCHEVFSNNPILQKVNNIKMRIEIFWELRELIEKTDLLNNEFSLSTLLLKYWYAIAIRYNLKEAIQYFYQKYTHLNTWRLTCALCFNNVFDLHEAYEKDKIHMDIEEMMRIACIKDHNLSTMYYCYVLGANINQAMLSSIQYYNIENMFFCIDLGADVFEEGTTALGEGYELIKNILSLKIYSPATTPLPKSMDPEIIDHALKNYVSKNMMIFLTYDLR

SEQ ID NO: 162 - protein, 350 aa, African swine fever virus
MLPSLQSLTKKVLAGQCVPTNQHYLLKCYDLWWHDGPITFDHNLKLIKSAGIKEGLDLNTALVKAVRENNYNLIKLFAEWGANINYGLVSVNTEHTRDLCRELGAKETLNEEEILQIFIDLKFHKTSSNIILCHEVFSNNPILQKVNNIKMRIEIFWELRELIEKTDLLNNEFSLSALLLKYWYAIAIRYNLKEAIQYFYQKYTHLNTWRLTCALCFNNVFDLHEAYEKDKIHMDIEEMMRIACIKDHNLSTMYYCYVLGANINQAMLTSIQYYNIENMFFCMDLGADAFEEGTIALGEGYKLIKNILSLKIYSPATTPLPKSTDPEIIDHALKNYVSKNMMIFLTYDLR

SEQ ID NO: 163 - protein, 350 aa, African swine fever virus
MLPSLQSLTKKVLAGQCVPTNQHYLLKCYDLWWHDGPITFDHNLKLIKSAGIKEGLDLNTALVKAVRENNYNLIKLFAEWGADINYGLVSVNTEHTRDLCRELGAKETLNEEEILQIFIDLKFHKTSSNIILCHEVFSNNPILQKVNNIKMRIEIFWELRELIEKTDLLNNEFSLSTLLLKYWYAIAIRYNLKEAIQYFYQKYTHLNTWRLTCALCFNNVFDLHEAYEKDKIYMDIEEMMRIACIKDHNLSTMYYCYVLGANINQAMLTSIQYYNVENMFFCMDLGADAFEEGTIALGEGYKLIKNILSLKIYSPATTPLPKSTDPEIIDHAVKNYVSKNMMIFLTYDLR

SEQ ID NO: 164 - protein, 350 aa, African swine fever virus
MLPSLQSLTKKVLAGQCVPTNQHYLLKCYDLWWHDGPITFDHNLKLIKSAGIKEGLDLNTALVKAVRENNYNLIKLFAEWGANINYGLVSVNTEHTWDLCRELGAKETLNEEEILQIFIDLKFHKTSSNIILCHEVFSNNPILQKVNNIKMRIEIFWELRELIEKTDLLNNEFSLSTLLLKYWYAIAIRYNLKEAIQYFYQKYTHLNTWRLTCALCFNNVFDLHEAYEKDKIHMDIEEMMRIACIKDHNLSTMYYCYVLGGNINQAMLTSIQYYNIENMFFCMDLGADAFEEGTIALGEGYKLIKNILSLKIYSPATTPLPKSTDPEIIDHALKNYVSKNMMIFLTYDLR

SEQ ID NO: 165 - protein, 350 aa, African swine fever virus
MLPSLQSLTKKVLAGQCVPTNQHYLLKYYDLWWHDAPITFDHNLRLIKSAGIKEGLDLNTALVKAVKENNYNLIKLFAEWGADINYGLVSVSSEHTWDLCRELGAKETLNEKEILQIFIDLKFHKTSSNIILCHEVFSNNPILQKVNNIKMRIEIFWELRELIEKTDLLNNEFSLSTLLLKYWYAIAIRYNLKEAIQYFYQKYTHLNTWRLTCALCFNNVFDLHEAYEKDKIYMDIEEMMRIACIKDHNLSTMYYCYMLGANINQAMLTSIQYYNIENMFFCMDLGADVFEEGTTALGEGYELIKNILSLKIYSPTTIPLPKSTDPEIIDHALKNYFSKNMMIFLSYDLR

SEQ ID NO: 166 - protein, 350 aa, African swine fever virus
MLPSLQSLTKKVLAGQCVPTNQHYLLKYYDLWWYNAPITFDHNLRLIKSSGIKEGLDLNTALVKAVRENNYSLIKLFTEWGADINYGLVSVNTEHTRDLCQELGAKEILNEEEILQIFIDLKFHKTSSNIILCHEVFSNNPILQKVNNLKLRIEIFWELRELIEKTDLLNNEFLLSTLLLKYWYAIAVRYSLKEAIQYFYQKYTHLNTWRLTCALCFNNVFDLHEAYEKDKIHMDIEEMMRIACIKDHNLSTMYYCYMLGANINQAMLTSIQYYNIENMFFCMDLGADVFEEGTTALGEGYELIKNILSLKIYSPTTIPLPKSTDPEIIDHALKNYFSKNMMIFLSYDLR

SEQ ID NO: 167 - protein, 349 aa, African swine fever virus
MLPSLQSLTKKVLAGQCLPTDQYYLLKCYDLWWYDSPITFDHNLGLIKSAGIKDGLDLNTALVKAVRENNYNLIKLFTEWGADINYGLVSVNTEHTRDLCRELGAKETLNEEEILRIFIDLKFYKTSSNIILCHEVFSNNPLLQKVNNLKMRIEIFWELRELIKKTDLLNNEFSLNTLLLKYWYAIAVRYNLKEAIQYFYQKYTHLNTWRLTCALCFNNVFDLHEAYEKDKIYMDLEEMMRVACIKDHNLSTIYYCYVLGANINQAMLASIQYYNIENMFFCMDLGADVFEENMPVGEGYELIRNILSLKIYSPSTAPLPKNTDPEIIDHVLKNYKSKNMMTFLSYDLR

SEQ ID NO: 168 - protein, 351 aa, African swine fever virus
MLPSLQSLTKKVLAGQCLPEDQHYLLKCYDLWWNNAPITFDHNLRLIKSAGLQEGLDLNMALVKAVKENNYSLIKLFTEWGANINYGLISVNTEHTWDLCRELGAKKTLNEGDILQIFIDLKFHKTSSNIILCHEVFSDNLLLKKVNNLKMRIEIFWELREIIEKTDLLNNEFSLNTLLLKYWYAIAVRYNLKEAIQYFYQKYTHLNTWRLTCALCFNNVFDLHEAYEKDKICMDLEEMMRIACIKDHSLSTIYYCYMLGANINQAMLTSIQYYNIENIFFCMDLGADAFEEGMALVGQEGYEPIRNILSLKIYSPATTPLPKSTDPEIIDHELKNYFSKNMMVFLTYDLR

SEQ ID NO: 169 - protein, 351 aa, African swine fever virus
MLPSLQSLTKKVLARQCLPEDQHYLLKCYDLWWNNAPITFDHNLRLIKLAGIQEGLDLNMALVKAVKENNYSLIKLFTEWGANINYGLISVNTEHTWDLCRELGAKKTLNEGDILQIFIDLKFYKTSSNIILCHEVFSDNLLLKRVNNLKMRIEIFWELREIIEKTDLLNNEFSLNTLLLKYWYAIAVRYNLKEAIQYFYQKYTHLNTWRLTCALCFNNVFDLHEAYEKDKICMDLEEMMRIACIKDHNLSTIYYCYMLGANINQAMLTSIQYYNIENIFFCMDLGADAFEEGMALVGQEGYEPIRNILSLKIYSPATTPLPKSTDPEIIDHALKNYFSKNMMVFLTYDLR

SEQ ID NO: 170 - protein, 353 aa, African swine fever virus
MSLPLSLQTLVKKTIASQCLSIDEHCILKYCGLWWHDAPLKLCMDRGRIQIKSGFLGEDIDLRVALIIAVKENNYSLIKLFTEWGANINYGLLSINTKHIRELCRQLGAKETLEDNDIFRIFTRIMHNKTSGSIILCHEIFMNNPILENKFVIQLRGLIYKRLWGLIEIKETDELNGLLVKYWYAKAVQYDCKDAICFLDEKYTDLNEWRLKCLLYYNKIYELHEMYHKENIQIDVHDMICLASTKDNNPLTIYYCYALGGNINQAMLTSVQYYNIGNIFFCIDLGGNAFEEGRAIAEQKGYNFLSHSLALDIYSSDASLPLNLKDPEEISSLLKDYKSKNLSIIWEYSHNIL

SEQ ID NO: 171 - protein, 353 aa, African swine fever virus; identical to SEQ ID NO: 170

SEQ ID NO: 172 - protein, 353 aa, African swine fever virus
MSLPLSLQTLVKKTVANQSLSIDEHCILKHCGLWWHDVPLKLCMDRGQIQIKSGFLGEDIDLHIALIIAVKENNYSLIKLFTEWGANINYSLLSINTKHIRELCRQLGAKETLEDDDIFRIFTKIMHNKTSGSIILCHDIFMNNPNIEDKFTIQLRGLIYKRLWGLIEIKETDELNGLLVKYWYAKAVQYECKDAICFLDEKYTDLNEWRLKCLLYYNKIYELHEMYHKENIQIDVHDMICLASTKDNNPLTIYYCYALGGNINQAMLTSVQYYNIGNIFFCIDLGGNAFEEGRAIAEQKGYNFLSHSLALDIYSSDASLPLNLKDPEEISSLLKDYKSKNLSIIWEYSHNIL

SEQ ID NO: 173 - protein, 353 aa, African swine fever virus
MSLPLSLQTLVKKTVASQCLSTDEHCILKHCGLWWHDVPLKLCMDRGQIQIKSGFLGEDIDLHIALIIAVKENNYSLIKLFTEWGAHINYSLLSINTEHIRELCRQLGAKETLEDNDIFRIFTKIMHNKTSGRIILCHEIFMNNPNIENKFTIQLRGLICKRLWGLIEIKETDELNDLLVKYWYAKAVQYECKDAICFLDEKYTDLNEWRLKCLLYYNKIYELHEMYHKEKVQIDVHDMICLASTKDNNPLTIYYCYALGGNINQAMLTSVQYYNIGNIFFCIDLGGNAFEEGRAIAEQKGYHFLSHSLTLDIYSSDASLPLNLKDPEEISSLLKDYKSKNLSIIWEYSHNIL

SEQ ID NO: 174 - protein, 353 aa, African swine fever virus
MSLPLSLQTLVKKTVASQCLSTDEHCILKHCGLWWHDVPLKLCMDRGQIQIKSGFLGEDIDLHVALIIAVKENNYSLIKLFTEWGAHINYSLLSINTEHIRELCRQLGAKKTLEDNDIFRIFTKIMHNKTSGRIILCHEIFMNNPNIENKFTIQLRGLICKRLWRLIEIKETDELNDLLVKYWYAKAVQYECKDAICFLDEKYTGLNEWRLKCLLYYNKIYELHEMYHKDKVQIDVHDMICLASTKDNNPLTIYYCYALGGNINQAMLTSVQYYNIGNIFFCIDLGGNAFEEGRAIAEQKGYHFLSHSLALDIYSSDASLPLNLKDPEEISSLLKGYKSKNLSIIWEYSHNIL

SEQ ID NO: 175 - protein, 353 aa, African swine fever virus
MSLPLSLQTLVKKTVASQCLSIDEHCILKHCGLWWHDVPLKLCMDRGQIQIKSGFLGEDIDLHIALIIAVKENNYSLIKLFTEWGAHINYSLLSINTEHIRELCRQLGAKETLEDDDVFRIFTKIMHNKTSGRIILCHDIFMNNPNIENKFTIQLRGLICKRLWGLIEIKETDELNDLLVKYWYAKAVQYECKDAICFLEEKYTDLNEWRLKCLLYFNKIYELHEMYHKEKVQIDVHDMICLASTKDNNPLTIYYCYALGGNINQAMLTSIQYYNIGNIFFCIDLGGNAFEEGRAIAEQKGYNFLSHSLALDIFSSDASLPLNLKDPEEISSFLKDYKSKNLSIIWEYSHNIL

SEQ ID NO: 176 - protein, 353 aa, African swine fever virus
MSLPLSLQTLVKKTVASQCLSIDEHCILKYCGLWWHDAPLKLCMDRGRIQIKSGFLGEDIDLRVALIIAVKENNYSLIKLFTEWGANINYSLLSINTKHIRELCRQLGAKETLEDNDIFRIFTRIMHNKTSGSIILCHEIFMNNPILENKFVIQLRGLIYKRLWGLIEIKETDKLNDLLVKYWYAKAVQYVCKNAICFLDEKYTDLNEWRLKCLLYYNKIYELHEMYHKKKVQIDVHDMICLACAKDNNLLTIYYCYALGSNINQAMLTSVQYYNIGNIFFCIDLGGNAFEEGRAIAEQKGYNFLSHSLTLDIYSSDASLPLNLKDPEEISSLLKDYKSKNLSIIWEYSHNIL

SEQ ID NO: 177 - protein, 353 aa, African swine fever virus; identical to SEQ ID NO: 7

SEQ ID NO: 178 - protein, 353 aa, African swine fever virus
MSAPLSLQTLVKKTVASTSCLSIDEHILKYCDLWWHDAPLKLYMDRGRIQIKSGFLGEDIDLCVALIIAVKENNYNLIKLFTELGANINYSLLSINTKHVRDLCRQLGAKETLEDYDIFCIFNKIMHNKTSGSVILCHEIFINNPNLENKFAAQLRRLIYKRLCGLIEIKETDELSELLVKYWYAKAVQYDYKDAICFLDEKYTDLNEWRLKCYLYYNKIYELHDIYHKEKIQIDVNEMLSLACIRDNNPLTIYYCYALGGNINQAMLTSVQYYNIGNIYFCIDLGGNAFEEGSAIARQKGYNFLSHSLVLNIYSSDASLPLNLKDPEEISSLLKNYKSKNLSIILDYSHNIL

SEQ ID NO: 179 - protein, 353 aa, African swine fever virus
MSSPLSLQTLVKKTVASTSCLSIDEHILKYCGLWWHDAPLKLYIDRGRIYIKSGFLGEDIDLCVALIIAVKENNYSLIKLFTEWGAYINYSLLSINTKHARDLCRQLGAKETLDDYDIFCIFNKIMHNKTSGSIILCHEIFINNPKLENNFAAQLRRLIYKRLCGLIEIKETDELSELLVKYWYANAVQYDHKDAICFLDEKYTDLDEWRLKCYLCYNKIYELHDIYHKKKIQIDVNEMLSLACIRDNNLLTIYYCYALGGNINQAMLTSVQYYNIGNIYFCIDLGGNAFEEGSAIARQNGYNFLCHSLILNIYSSDASLPLNLKVPEEISSLLKNYKSKNLSIILDYSHKIL

SEQ ID NO: 180 - protein, 352 aa, African swine fever virus
MSSPLSLQTLVKKTVAGTSCLSIDEHILKYCGLWWHDAPLKLYIDRGRIYIKSGFLGEDIDLCVALIIAVKENNYSMIKLFTEWGAYINYSLLSINTKHARDLCRQLGAKETLDDYDIFCIFNKIMHNKTSGSIILCHEIFINNPNLENNFAAQLRRLIYKRLCGLIEIKETDELSELLVKYWYANAVQYDHKDAICFLDEKYTDLDEWLKCYLCYNKIYELHDIYHKEKIQIDVNEMLSLACIRDNNLLTIYYCYALGGNINQAMLTSVQYYNIGNIYFCIDLGGNAFEEGSAIARQNGYNFLSHSLVLNIYSSDASLPLNLKDPEEISSLLKNYKSKNLSIILDYSHKIL

SEQ ID NO: 181 - protein, 357 aa, African swine fever virus
MLSLQTLAKKVVACNYLSSDYDYTLQRFGLWWDLGPIHLCNNCKQVFSYKHLQCFSEDDLCLEAALVKAVKSDNLELIRLFVDWGANPEYGLIRVPAVYLKRLCAELGGLTPVSEPRLLEILKEVARLKSCAGVLLGYDMFCHNPLLETVTRTTLDTVTYTCSNIPLTGDTAHHLLTKFWFALALRHNFTKAIHYFYKRHKNHLYWRVACSLYFNNIFDIHELCREKEICISPNLMMKFACLREKNYAAIYYCHRLGASLDYGMNLSIYNNNTLNMFFCIDLGAADFDRAQLIAHKAYMYNLSNIFLVKQLFSRDVTLVLDVTEPQEIYDMLKTYTSKNLKRAEEYLTAHPEIIVID

SEQ ID NO: 182 - protein, 357 aa, African swine fever virus; identical to SEQ ID NO: 181

SEQ ID NO: 183 - protein, 357 aa, African swine fever virus
MLSLQTLAKKVVACNYLSSDYDYTLQRFGLWWDLGPIHLCNNCKQIFSYKHVQCFSEDDLCLEAALVKAVKSDNLELIRLFVDWGANPEYGLIRVPAVHLKRLCTELGGLTPVSESRLLEILKEVARLKSCAGVLLGYDMFCHNPLLETVTRTTLDTVTYTCSNIPLTGDTAHHLLTKFWFALALRHNFTKAIHYFYKRHKNHLYWRVACSLYFNNIFDIHELCREKEICISPNLMMKFACLQKKNYAAIYYCYRLGASLDYGMNLSIYNNNTLNMFFCIDLGATDFDRAQRIAHKAYMYNLSNIFLVKQLFSRDVTLALDVTEPQEIYDMLKSYTSKNLKRAEEYLTAHPEIIVID

SEQ ID NO: 184 - protein, 357 aa, African swine fever virus
MLSLQTLAKKVVACNYLSSDYDYMLQRFGLWWDLGPIHLCNNCKQIFSYKHLQCFSEDDLCLEAALVKAVKSDNLELIRLFVDWGANPEYGLIRVPAVHLKRLCTELGGLTPVSEPRLLEILKEVAKLKSCAGVLLGYDMFCHNPLLETVTRTTLDTVTYTCSNIPLTGDTAHHLLTKFWFALALRHNFTKAIHYFYKRHKNHLYWRVACSLYFNNIFDIHELCREKEICISPNLMMKFACLRKKNYAAIYYCYRLGASLDYGMNLSIYNNNTLNMFFCIDLGATDFDRAQRIAHKTYMYNLSNIFLVKQLFSRDVTLALDVTEPQEIYDMLKSYTSKNLKRAEEYLTAHPEIIVID

SEQ ID NO: 185 - protein, 357 aa, African swine fever virus
MLSLQTLAKKVVACNYLSSDYDYMLQRFGLWWDLGPIHLCNNCKQIFSYKHLQCFSEDDLCLEAALVKAVKRDNLELIRLFVDWGANPEYGLICVPTVHLKRLCTELGGLTPVSEPRLLEILKEVANLKSCAGVLLGYDMFCYNPLLETITRTTLDTVMYSCSKIPLMGDTAHHLLTKFWFALALRHNFTKAIHYFYKRHKNHLYWRVACSLYFNNIFDIHELCREKEICISPNLMMKFACLREKNYAAIYYCYRLGASLDYGMNLSIYNNNSLNLFFCIDLGATDFDRAQRIAHKAYMYNLSNILLVKQLFSRDVTLALDVTEPQEIYDRLKAYTSKNMKRAEEYLTAHPEIIVID

SEQ ID NO: 186 - protein, 357 aa, African swine fever virus
MLSLQTLAKKVVACNYLSSDYDYTLQRFGLWWDLGPIHLCNNCKQIFSYKHLQCFSEDDLCLEAALVKAVKSDNLELIRLFVDWGANPEYGLIRVPAVHLKRLCTELGGLTPVTEPRLLEILKEVAKLKSCAGVLLGYDMFCHNPLLETVTRTTLDTVTYTCSNIPLTGDTAHLLLTKFWFALALRHNFTKAIHYFYKRHKNHLYWRVACSLYFNNIFDIHELCREKEICISPNLMMKFACLQKKNYAAIYYCYRLGASLDYGMNLSIYNNNTLNMFFCIDLGATDFDRAQHIAHKAYMYNLSNIFLVKQLFSRDVTLALDVTEPQEIYDRLKSYTSKNLKRAEEYLTAHPEIIVID

SEQ ID NO: 187 - protein, 357 aa, African swine fever virus; identical to SEQ ID NO: 8

SEQ ID NO: 188 - protein, 357 aa, African swine fever virus; identical to SEQ ID NO: 8

SEQ ID NO: 189 - protein, 357 aa, African swine fever virus
MLSLQTLAKKVVACNYLSSDYDYTLQRFGLWWDLGPIHLCNTCKQIFSYKHLQCFSEDDLCLEAALVKAVKSDNLELIRLFVDWGANPEYGLIRVPAVHLKRLCMELGGLTPVSESRLLEILKEVADLKSCAGVLLGYDMFCHNPLLETVTRTTLDTVMYTRSKIPLTGDTAHLLLSKFWFALALRHNFTKAIHYFYEKHKNQLYWRLTCSLYFNNIFDLHELCCKKEICISPNLMMKFACLREENYAAIYYCHMLGASLDYGMNLSIYNNNTLNLFFCIDLGATNFDRARLIARRVYMYNLSNLFLVKQLFSRDVSLILDLTEPQAIYDMLNTYTSKNLKQAEEYFTAHPEIVVID

SEQ ID NO: 190 - protein, 357 aa, African swine fever virus
MLSLQTLAKKVVACNYLSSDYDYTLQRFGLWWDLGPIHLCNTCKQIFSYQHLQCFSEDDLCLDAALVKAVKSDNLELIRLFVDWGANPEYGLIRVPAVDLKRLCTELGGLTPVSESRLLEILKEVADLKSCAGVLLGYDMFCHNPLLETVTRTTLNTVMYTRSKIPLTGDTAHLLLTKFWFALALRHNFTKAIHYFYEKHKNQLYWRLACSLYFNNIFDLHELCSKKEICISPNLMMKFACLREENYAAIYYCHMLGASPDYGMNLSIYNNNTLNLFFCIDLGATNFDRARLIAHRVYMYNLSNIFLVKQLFSRDASLVLNITEPQAIYDMLKTYTSKNLKRAEEYFTAHPEIVVID

SEQ ID NO: 191 - protein, 357 aa, African swine fever virus
MLSLQTLAKKVVACNYLSSDYDYTLQRFGLWWDLGPIHLCNTCKQIFSYQHLQCFSVDDLCLDAALVKAVKSDNLELIRLFVDWGANPEYGLIRVPAVHLKRLCTELGGLTPVSESRLLEILKEVADLKSCAGVLLGYDMFCHNPLMETVTRTTLNTVMYTRSKIPLTGDTAHLLLTKFWFALALRHNFTKAIHYFYEKHKNQLYWRLTCSLYFNNIFDLHELCSKKEICISPNLMMKFACLREENYAAIYYCHMLGASPDYGMNLSIYNNNTLNLFFCIDLGATNFDRARLIAHRVYMYNLSNIFLVKQLFSRDASLVLNITEPQAIYDMLKTYTSKNLKRAEEYFTAHPEIVVID

SEQ ID NO: 192 - protein, 531 aa, African swine fever virus
MFSLQNLCRKTLPNRKLPEFFDEYILQLLGLYWENHGTIQRAGNNCVLIQQHTLIPVNEALRTAASEENYEIVSLLLAWEGNLYYAIIGALEGNRHDLIRKYDDQIKDHHEILPFIDDPVIFHKCHIMRQCFFDCILYQAVKYSKFRVLLYFKHRLEDDLPFTHLLIEKACKDHNYEVIKWIYENLHIYNMIDTFECAIAHKDLHLYCLGYRFIYNRIVPDKYHHLDIRMLSSLQLLHKVAAKGYLDFILETLKYDHNKDNINIILTQAATYNHRKILIYFIPQSTHAQIEQCLLVAIKAKSSRKTLNLLLSHLNLSINLIKKISHYVATYNSTNIIGILSMRRKKKIYLDIILTKFVKKAIFNKFVVRCMDTFSINPERILKIAARINRMMLVKKISEHVWKNHAVRLKYLKHAVHTMKHKDGKNRLMNFIYDRCYYHMQGEEIFSLARFYAIHHAPKLFDVFYDCCILDTIRFKSLLLDCSHIIGKNAHDATNINIVNKYIGNLFVMGVLSKKEILQDYPSIYSKQYMP

SEQ ID NO: 193 - protein, 531 aa, African swine fever virus; identical to SEQ ID NO: 192

SEQ ID NO: 194 - protein, 531 aa, African swine fever virus
MFSLQNLCRKTLPDCKLPEFFDEYILQLLGLYWENHGTIQRAGNNCVLIQQHNLIPVNEALRIAASEENYEIVSLLLAWEGNLYYAIIGALEGNRHDLIRKYDDQIKDHHEILPFIDDPVIFHKCHIMRQCFFDCILYQAVKYSKFRVLLSFKHRLRDDLPFTHLLIEKACKDHNYEVIKWIYENLHIYNMIDTFECAIAHKDLRLYCLGYRFIYNRIVPDKYHHLDIRMLSSLQLLHKVAAKGYLDFILETLKYDHNKDNINIILTQAATYNHRKILIYFIPQSTHAQIEQCLLVAIKTKSSKKTLNLLLSHLNLSINLIKKISHYVATYNSTNIIGILSMRRKKKIYLDIILTKFVKKAIFNKFVVRCMDTFSINPERILKIAARINRTMLVKKISEHVWKNHAVRLKYLKHAVHTMKHKDGKNRLMNFIYDRCYYHMQGEEIFSLARFYAIHHAPKLFDVFYDCCILDTIRFKSLLLDCSHIIGKNAHDATNINIVNKYIGNLFVMGVLSKKEILQDYPSIYSKHYMP

SEQ ID NO: 195 - protein, 531 aa, African swine fever virus
MFSLQNLCRKTLPDCKLPEFFDEYILQLLGLYWENHGTIQRAGNNCVLIQQRTLIPVNEALRIAASEENYEIVGLLLAWEGNLYYAIIGALEGNRHDLIRKYDDQIKDHHEILPFIDDPVIFHKCHIIRRCFFNCILYQAVKYSKFRVLLYFKHRLEDDLPLTHLLIEKACEDHNYEVIKWIYENLHTYDIMDTFECAIAHKDLRLYCLGYTFIYNRIVPYEYHHLDILILSSLQLLHKVAAKGYLDFILETLKYDHNNDNLDIILTQAATYNHRKILTYFIPQLTYAQIEQCLFMAIKRKSSKKTLNLLLSHLTLSIELIKKISQYVVTYNSTNIIGILSMKRKKKIYLDIMLTKYVKYAIFNKYVVRCMDRFSINPERIIKMAARINRMMLVKKISEHVWKNHAARLKHLKHAVHTMKHKDGKNRLMNFIYDRCYYHMQGEEIFSLARFYAIHHAPKLFDVFYDCCILDTIQFKSLLLDCSHIIGKNAHDATNINIVNKYIGDLFAMGVLSKKEILQDYPSIYSKHYML

SEQ ID NO: 196 - protein, 531 aa, African swine fever virus
MFSLQNLCRKTLPDCKLPEFFDDYILQLLGLYWENHGTIQRAGNNCVLIQQHTLIPINEALRTAASEENYEIVSLLLAWEGNLYYAIIGALEGNRHDLIRKYDDQIKDHHEILPFIDDPVIFHKCHIMRRCFFDCILYQAVKYSKFRVLLYFKYRLEDDLPLTHLLIEKACENHNYEVIRWIYENLHIYNMIDTFECAIAHKDLRLYCLGYTFIYNRIVPNKYHHIDILILSSLQLLHKVAAKGYLDFILETLKYDHNNDNLDIILTQAATYNHRKILTYFIPQSTYAQIEQCLMVAIKTKSSKKTLNLLLSHLNLSIKLIKKISQYVVTYNSTNIIGILSMKRKKKIYLDILLTKFVKNAIFNKFVVRYMDTFSINPEKIVKMAARINKMMLVKKISEHIWKNHAARLEHLKHAVHTMKHKDGKNRLMNFIYEYCYYHMQGEEIFSLARFYAIHHAPKLFDVFYNCCILDTIRFKSLLLDCSHIIGKNAHDATNINIVNKYIDNLFAMGVLSKKEILQDYPSIYSKHYMP

SEQ ID NO: 197 - protein, 531 aa, African swine fever virus
MFSLQNLCRKTLPDCKLPEFFDEYILQLLGLYWENHGTIQRAGNNCVLIQQHTLIPVNEALRIAASEENYEIVSLLLAWEGNLYYAIIGALEGNRHDLIRKYDDQIKDHHEILPFIDDPVIFHKCHIMRRCFFDCILYQAVKYSKFRVLLYFKYRLENDLPLAHLLIKKACEDHNYEVIKWIYENLHIYNIMDTFGCAIAHKDLRLYRLGYTFIYNRIVPYKYHYLDVLILSGLHLLYKVAAKGYLDFILETLKYDHNNDNLDIILTQAATYNHRKILTYYIPQLTYAQIEQCLFMAIKKKSSKKTLNLLLSHLKLSIKLIKKISQYVATYNSTNIIGILNMRRKKKIYLDIILTKFVKKAIFNKFVVRCMDTFSINPERIIKMAARINKMLLVKKISEHAWKNHAARLKHLKHAVYTMKHKDGKNRLMNLIYDHYYYHMQGEEIFSLARFYAIHHAPKLFDVFYDCCLLDTIRFKNLLLDCSHIIGKNAHDATNITIVNKYIGNLFAMGVLSKKEILQDYPSIYSKHYMP

SEQ ID NO: 198 - protein, 531 aa, African swine fever virus; identical to SEQ ID NO: 9

SEQ ID NO: 199 - protein, 531 aa, African swine fever virus
MFSLQNLCRKTLPDCKLPEFFDEYILQLLGLYWENHGTIQRAGNNCVLVQQHTLIPVNEALRIAASEENYEIVSLLLAWEGNLYYAIIGALEGNRHDLIRKYDDQIKDHHEILPFIDNPVIFHKCHIMRRCFFDCILYQAVKYSKFRVLLYFKYRLENDLPLAHLLVEKACEDHNYEVIKWLYENLHIYNIMETFECAIAHKDLRLYRLGYTFIYNRIVPYKYHYLDVLILSGLHLLYKVAAKGYLDFILETLKYDHNNDNLDIILTQAVTYNHRKILTYYIPQLTYAQIEQCLFMAIKKKSSKKTLNLLLSHLKLSIKLIKKISQYVATYNSTNIIGILNMKRKKKIYLDIILTKFVKYAIFNKYVVRCMDTFSINPERIIKMAARINKMLLVKKISQHAWKNHAARLKHLKHAVYTMKHKDGKNRLMNLIYDHYYYHMQGEEIFSLARFYAIHHAPKLFDVFYDCCLLDTIRFKSLLLDCSHIIGKNAHDATNITIVNKYIGNLFAMGVLSKKEILQDYPSIYSKHYMP

SEQ ID NO: 200 - protein, 530 aa, African swine fever virus
MFSLQNLCRKTLPDRKLPEFFDDYVLQLLGLYWENHGTIQRAGNNCVLIQQHNLIPVNEALRIAASEENYEIVSLLLAWEGNLYYAIIGALEGNRHNLIRKYDDQIKDHHEILPFIDDPVIFHKCHMMRRCFFNCILYQAVKYSKFSVLLYFKYILKENLPLVHSLIEKACEDHNYEVIKWIYENLHIYEIMDTFKCAIAHKDLHLYSLGYTFIYNRIVPYKYHHLDIRILSRLQLLHKVTAKGYLDFILETLKYDHNKDNINIILTQAATYNHRNILTYFIPQSTYAQIEQCLFVAIKTNASKKTLNLLLSHLNLSIKLVKKLSQYVVAYKSTNIISILSMQQKKKIYLDIILTKVVKNAIFIKFVIGCMVTFSINPERIVKMAARIKKMKLVKNISEHVWKNHAAKLKHLKHAVHTMKHQEGKNRLMNFIYDHCYYHMQGEEIFSLARFYAIHHAPKLFDVFYDCCILDTIRFKSLLLDCSYIIAKNAHDASINIVNKYIGNLFAMGVLSKKEILQDYPSIYSKDYML

SEQ ID NO: 201 - protein, 530 aa, African swine fever virus
MFSLQNLCRKTLPDCKLPEFFDDYVLQLLGLYWENHGTIQRAGNNCVLIQQHNLIPVNEALRIAASEENYEIVSLLLAWEGNLYYAIIGALEGNHHNLIRKYDDQIKDHHEILPFIDDPVIFHKCHTMRRCFFNCILYQAVKYSKFSVLLYFKYILKENLPLVHSLIEKAYKYHNYEVIKWIYENLHIYDIINTFKCAIAHKDLRLYCLGYTFVYNRIVPYKYYHLDIRILLRLQLLHKVTAKGYLDFILETLKYDHNTNNIDIILTQAATYNHRNILTYFIPQSTYAQIEQCLFVAIKTNASKKTLNLLLSHLNLSIKLVKKLSQYVVAYKSTNIISILSRQQKKKIYLDIILTKVVKNAVFNKFVIGCMVTFSINPERIVKMAARIKKMKLVKNISEHVWKNHAVKLKYLKHAVHTMKHQEGKNRLMNFIYDHCYYHMQEEEIFSLARFYAIHHAPKLFDVFYDCCILDTIRFKSLLLDCSHIIVKNAHDASINIVNKYIGNLFAMGVLSKKEILQDYPSIYSKDYML

SEQ ID NO: 202 - protein, 530 aa, African swine fever virus
MFSLQNLCRKTLPDCKLPEFFDDYVLQLLGLYWENHGTIQRAGNNCVLIQQHNLIPVNEALRIAASEENYEIVSLLLAWEGNLYYAIIGALEGNRHNLIRKYDDQIKDHHEILPFIDDPVIFHKCHIMRRCFFNCMLYQAVKYSKFSVLLYFKYILKENLPLVHSLIEKAYKYHNYEVIKWIYENLHIYDIINTFKYAIAHKDLRLYCLGYTFVYNRIVPYKYYHLDIRILLRLQLLHKVTAKGYLDFILETLKYDHNTNNIDIILTQAATYNHRNILTYFIPQSTYAQIEQCLFVAIKTNASKKTLNLLLSHLNLSIKLVKKLSQYVVAYKSTNIISILSMQQKKKIYLDIILTKVVKNAVFIKFVIGCMVTFSINPERIVKMAARIKKMKLVKNISEHVWKNHAAKLKHLKHAVHTMKHQEGKNRLMNFIYDHCYYHMQGEEIFSLARFYAIHHAPKLFDVFYDCCILDTIRFKSLLLDCSHIIAKNAHDASINIVNKYIGNLFAMGVLSKKEILQDYPSIYSKYDIL

SEQ ID NO: 203 - protein, 530 aa, African swine fever virus
MFSLQNLCRKTLPDCKLPEFFDEYILQLLGLYWENHGTIQRAGNNCVLIQQHNLIPVNEALRIAASEENYEIVSLLLAWEGNLYYAIIGALEGNRPDLIRKYDDQIKDHHEILPFIDDPIIFHKCHIMRRCFFNCILYQAVKYSKFRVLLYFKHRLGDDLPLTHLLIEKACEDHNYEVIKWIYENLHSYNIMDTFECAIAHKDLRLYCLGYTFIYNRIVPYKYHHLDICILSSLQLLHKVAAKGYLDFILETLKYDHNINNIDIILTQAATYNHRKILTYFIPQLTYAQIEQCLLVAIKTKASKKTLNLLLSHLNLSIKLIKKISQYVVTYNSTNIISILSMRRKKKIYLDIILTEFVKNAIFNKFVVRCMDTFSINPERIVKMAARINRMMLVKNISERVWKNHAVKLKHLKHAVHTMKHQEGKNRLMNFIYDHCYYHMQGEEIFGLARFYAIHHAPKLFDVFYDCCMLDATRFKSLLLDCPHIIGKNAYDAGINLVNKYIGNLFAMGVLSKKEILQDYPSIYSKHDMF

SEQ ID NO: 204 - DNA, 462 bp, African swine fever virus
atgtattttaagaaaaaatacatcggtcttattgataagaactgtgaaaaaaaaatattagatgattctagtacaataaaaatttgttacatattaattggaatattgattggaactaatatgataactcttatttataatttcatattctgggataattatataaaatgttaccgaaataatgataaaatgttttactgtcctaatgattgggttggatataataatatttgttactattttagtaatggtagtttttctaaaaattatacagctgctagtaatttttgtagacaattaaatggtacacttgctaataatgatactaatttattaaatctaactaaaatatataataatcaatctatgtattgggttaacaatacggtaatattacgtggtgataataaatatagtcaaaaagttaactatacagatttattatttatttgtggtaaataa

SEQ ID NO: 205 - DNA, 477 bp, African swine fever virus
atgttttctaacaaaaagtacatcggtcttatcaataagaaggagggtttgaaaaaaaaaatagatgattatagtatattaataattggaatattaattggaactaacatcttaagccttattataaatataataggagagattaataaaccaatatgttaccaaaatgatgataagatattttattgccctaaagattgggttggatataataatgtttgttattattttggcaatgaagaaaaaaattataataatgcaagtaattattgtaagcaattaaatagtacgcttactaataataatactattttagtaaatcttactaaaacattaaatcttactaaaacatataatcacgaatctaattattgggttaattattctttaattaaaaatgagtcagtactattacgtgatagtggatattacaaaaaacaaaaacatgtaagtttattatatatttgtagtaaataa

SEQ ID NO: 206 - DNA, 495 bp, African swine fever virus
atgtattctaagaaaaagtacatcggtcttattgacaagaactgtgaaaaaaaaatattagatgatgctactacaataaaaatttgttacatattaattggaatattgattggaactaatattataactcttatctataatttcatattttgggagaattatataaaatgtatccaaaatgatgataagatgttttactgtcctaaagattgggttgggtataataatgcgtgttattattttagtaataacaataaaaattatacagatgcaaataattattgtaaaaattcacataatagtacacttgctaataatgatactaaattattaaatcttactaaattattaaatctttctaaattatattacaatgattctacatattgggttaaatattctttacctaaaaataaagcagtaacattacgtaacagtacctacaaatatgatagagttaaatctacagaaacattttttatttgtagtaactaa

SEQ ID NO: 207 - DNA, 498 bp, African swine fever virus
atgtattctaagaaaaagtacatcggtcttattgacaaaagctgtgaaaaaaaaatattagatgatcctactacaataaaaatttgttacatattaattggaatattgattggaactaatattataactcttatctataatttcatattttgggagaattatataaaatgtatccgaaatgatgataagatgttttactgccctaaagattgggttggatataataatgtgtgttattattttagtaacgataatggaaaaaattatatgaatgcaagtaatttttgtaataaaaaaaatagcacgcttgctaataatacgactaatttagtaaatcttactaaaatattaaattttaccaaacaatataatcatgaatcactttattgggttaattattctttaagtaataatatgtcattagcattacagaatagtaattttccttcttcaggaagacgtaaatatatggacttattatatatatgtagtatataa

SEQ ID NO: 208 - DNA, 462 bp, African swine fever virus; identical to SEQ ID NO: 204

SEQ ID NO: 209 - DNA, 477 bp, African swine fever virus; identical to SEQ ID NO: 205

SEQ ID NO: 210 - DNA, 486 bp, African swine fever virus
atgttttctaacaaaaagtacatcggtcttattgacaagtactgtgaaaaaaaaatattagatgattctagtacaataaaaatttgttacatattaattggaatactgattggaactaatatgataactcttatttataatttcatattttgggagaattatataacatgtaaccaaaaagataagacgttttactgtcctaaagattgggttggatataataatgtttgttattattttggcaatgatgaaaaaaattataataatgcaagtaattattgtaagcaattaaatagtacgcttactaataataatactaatttagtaaatcttactaaaacattaaatcttactaaaacatataatcacgaatctaattattgggttaattattctttaattaaaaatgagtcagtactattacgcaatagtggatattataaaaaacaaaaacatgtaagtttattatatatttgtagtaaataa

SEQ ID NO: 211 - DNA, 492 bp, African swine fever virus
atgttttctaataaaaagtacattggtcttatcaataagaaggagggattgaaaaaaaaaatagatgattatagtatattaataattggaatattaattggaactaacatcttaagccttattataaatataataggagagattaataaaccaatatgttaccaaaataatgataagatattttattgccctaaagattgggttggatataataatgtttgttattattttagtaatgataatggcaataattatacaactgcggataataaatgtaaacaattaaataatagtacactggctaataatcttactgatttattaaatcttacgagtttcttaaatcttactaaattatatcatcatcattctcattattgggttaattattctttaaataataattattcagtaccattaattgatagtaaatataatttaaataggaaaaaaagtcactatacagatttactatttatttgcagcaaataa

SEQ ID NO: 212 - DNA, 465 bp, African swine fever virus
atgtattttaagaaaaaatacatcggtcttattgataagaactgtgaaaaaaaaatattagatgattctagtacaataaaaatttgttacatattaattggaatattgattggaactaatatgataactcttatttataatttcatattctgggataattatataaaatgttaccgaaataatgataaaatgttttactgtcctaatgattgggttggatataataatgtttgttactattttagtaatggtagtttttttaaaaattatacagatgctaataattattgtaaaaaaataaatagtactcttgctaataatgatactaatttattaagtttaactaaaatatacaataatcaatctatgtattgggttaacaatacagtagcattacgtggtgatagtgtatatgcacaaagaaaagttaataattcagatctattatttatttgtagtaaataa

SEQ ID NO: 213 - DNA, 465 bp, African swine fever virus
atgtattttaagaaaaagtacatcggtcttattgataagaactgtgaaaaaaaaatattagatgattgtactacaataaaaatttgttacatattaattggaatattgattggaactaatatgataactcttatttataatttcatattttgggatcattatatgacatgtaacaaaaaagataagatgttttactgtcctaaagattgggttggatataataatgtttgttattattttagtaatgatagttttactaagaattatacatatgcaagtaattcttgtaaaaaaataaatagtacacttgctaataatgatactaatttactaaatcttattaaagtatataataatgtatctgagtattgggttaataatacagtaacattacgtggtgatcataaatacataaataagcaaaataattgtaatacagaatttttatttatttgtagtagatag

SEQ ID NO: 214 - DNA, 471 bp, African swine fever virus
atgttttctaacaaaaagtatccgagtcttatcgaaaaaaaaatggatgacttgatgactttaaagttttgttacatcattattgcaatcttgatcatgactaatatttttagccttgttgtgaatatatggggggggggtgatcgacaatcttatgagaatgtattttattgtcctaaagattgggttggatataataatgcatgttattattttagtaatgatatgaaaaattatacagaagcaagtaattattgtaaaaatttatataatagtacaattgttaataataatactaatatagtaaatcttactaaaacattaaatcttactaaaatatataatcatgaaagtaattattgggttaattattctttaattaataatgagtcattaacattacgggacagtaattttccttcttcaggaaaacgtacatatatagatttattatatatatgtagtatataa

SEQ ID NO: 215 - DNA, 468 bp, African swine fever virus
atgtattttaagaaaaagtacatcggtcttattgataagaactgtgaaaaaaaaatattagatgattgtactacaataaaaatttgttacatattaattggaatattgattggaactaatatgataactcttatttataatttcatattttgggatcattatatgacatgtaacaaaaaagataagatgttttactgtcctaaagattgggttggatataataatgtttgttattattttaataatgatagtaagaattatacaactgctactaattcttgtaaacaattaaatagtacacttgctaataatgatactaatttattaaatcttactaaagtatatcaccatgataaattatattgggttaactattctttaaatgacaatttttcattatcattacgtaatagtacatatgaaaaaagatctaaatatttgccattattatttatttgcagtaaataa

SEQ ID NO: 216 - DNA, 477 bp, African swine fever virus
atgtttttgaacaaaaagtatccgagtcttatcgaaaaaaaaatggatgatttgatgactttaaagttttgttacttaataatcacatttttaatcataaccaatatttttagccttgctataaatatatggggggggggtgatatgatagatcgacaaagttgtgaaaatatattttactgtcctaaagattgggttggctataataatgcgtgttattattttagtaataacaataaaaattatacagatgcaaataattattgtaaaaattcacataatagtacacttgctaataatgatactaaattattaaatcttactaaattattaaatctttctaaattatattacaatgattctacatattgggttaaatattctttacctaaaaataaagcagtaacattacgtaacagtacctacaaatatgatagagttaaatatacagaaacattttttatttgtagtaactaa

SEQ ID NO: 217 - protein, 158 aa, African swine fever virus
MFSNKKYIGLINKKEGLKKKIDDYSILIIGILIGTNILSLIINIIGEINKPICYQNDDKIFYCPKDWVGYNNVCYYFGNEEKNYNNASNYCKQLNSTLTNNNTILVNLTKTLNLTKTYNHESNYWVNYSLIKNESVLLRDSGYYKKQKHVSLLYICSK

SEQ ID NO: 218 - protein, 164 aa, African swine fever virus
MYSKKKYIGLIDKNCEKKILDDATTIKICYILIGILIGTNIITLIYNFIFWENYIKCIQNDDKMFYCPKDWVGYNNACYYFSNNNKNYTDANNYCKNSHNSTLANNDTKLLNLTKLLNLSKLYYNDSTYWVKYSLPKNKAVTLRNSTYKYDRVKSTETFFICSN

SEQ ID NO: 219 - protein, 165 aa, African swine fever virus
MYSKKKYIGLIDKSCEKKILDDPTTIKICYILIGILIGTNIITLIYNFIFWENYIKCIRNDDKMFYCPKDWVGYNNVCYYFSNDNGKNYMNASNFCNKKNSTLANNTTNLVNLTKILNFTKQYNHESLYWVNYSLSNNMSLALQNSNFPSSGRRKYMDLLYICSI

SEQ ID NO: 220 - protein, 153 aa, African swine fever virus; identical to SEQ ID NO: 20

SEQ ID NO: 221 - protein, 158 aa, African swine fever virus; identical to SEQ ID NO: 217

SEQ ID NO: 222 - protein, 161 aa, African swine fever virus
MFSNKKYIGLIDKYCEKKILDDSSTIKICYILIGILIGTNMITLIYNFIFWENYITCNQKDKTFYCPKDWVGYNNVCYYFGNDEKNYNNASNYCKQLNSTLTNNNTNLVNLTKTLNLTKTYNHESNYWVNYSLIKNESVLLRNSGYYKKQKHVSLLYICSK

SEQ ID NO: 223 - protein, 163 aa, African swine fever virus
MFSNKKYIGLINKKEGLKKKIDDYSILIIGILIGTNILSLIINIIGEINKPICYQNNDKIFYCPKDWVGYNNVCYYFSNDNGNNYTTADNKCKQLNNSTLANNLTDLLNLTSFLNLTKLYHHHSHYWVNYSLNNNYSVPLIDSKYNLNRKKSHYTDLLFICSK

SEQ ID NO: 224 - protein, 154 aa, African swine fever virus
MYFKKKYIGLIDKNCEKKILDDSSTIKICYILIGILIGTNMITLIYNFIFWDNYIKCYRNNDKMFYCPNDWVGYNNVCYYFSNGSFFKNYTDANNYCKKINSTLANNDTNLLSLTKIYNNQSMYWVNNTVALRGDSVYAQRKVNNSDLLFICSK

SEQ ID NO: 225 - protein, 154 aa, African swine fever virus
MYFKKKYIGLIDKNCEKKILDDCTTIKICYILIGILIGTNMITLIYNFIFWDHYMTCNKKDKMFYCPKDWVGYNNVCYYFSNDSFTKNYTYASNSCKKINSTLANNDTNLLNLIKVYNNVSEYWVNNTVTLRGDHKYINKQNNCNTEFLFICSR

SEQ ID NO: 226 - protein, 156 aa, African swine fever virus
MFSNKKYPSLIEKKMDDLMTLKFCYIIIAILIMTNIFSLVVNIWGGGDRQSYENVFYCPKDWVGYNNACYYFSNDMKNYTEASNYCKNLYNSTIVNNNTNIVNLTKTLNLTKIYNHESNYWVNYSLINNESLTLRDSNFPSSGKRTYIDLLYICSI

SEQ ID NO: 227 - protein, 155 aa, African swine fever virus
MYFKKKYIGLIDKNCEKKILDDCTTIKICYILIGILIGTNMITLIYNFIFWDHYMTCNKKDKMFYCPKDWVGYNNVCYYFNNDSKNYTTATNSCKQLNSTLANNDTNLLNLTKVYHHDKLYWVNYSLNDNFSLSLRNSTYEKRSKYLPLLFICSK

SEQ ID NO: 228 - protein, 158 aa, African swine fever virus
MFLNKKYPSLIEKKMDDLMTLKFCYLIITFLIITNIFSLAINIWGGGDMIDRQSCENIFYCPKDWVGYNNACYYFSNNNKNYTDANNYCKNSHNSTLANNDTKLLNLTKLLNLSKLYYNDSTYWVKYSLPKNKAVTLRNSTYKYDRVKYTETFFICSN

SEQ ID NO: 229 - DNA, 1083 bp, African swine fever virus
atgataatacttatttttttaatattttctaacatagttttaagtattgattattgggttagttttaataaaacaataattttagatagtaatattactaatgataataatgatataaatggagtatcatggaatttttttaataattcttttaatacactagctacatgtggaaaagcaggtaacttttgtgaatgttctaattatagtacatcaatatataatataacaaataattgtagcttaactatttttcctcataatgatgtatttgatacaacatatcaagtagtatggaatcaaataattaattatacaataaaattattaacacctgctactcccccaaatatcacatataattgtactaattttttaataacatgtaaaaaaaataatggaacaaacactaatatatatttaaatataaatgatacttttgttaaatatactaatgaaagtatacttgaatataactggaataatagtaacattaacaattttacagctacatgtataattaataatacaattagtacatctaatgaaacaacacttataaattgtacttatttaacattgtcatctaactatttttatactttttttaaattatattatattccattaagcatcataattgggataacaataagtattcttcttatatccatcataacttttttatctttacgaaaaagaaaaaaacatgttgaagaaatagaaagtccaccacctgaatctaatgaagaagaacaatgtcagcatgatgacaccacttccatacatgaaccatctcccagagaaccattacttcctaagccttacagtcgttatcagtataatacacctatttactacatgcgtccctcaacacaaccactcaacccatttcccttacctaaaccgtgtcctccacccaaaccatgtccgccacccaaaccatgtcctccacctaaaccatgtccttcagctgaatcctattctccacccaaaccactacctagtatcccgctactacccaatatcccgccattatctacccaaaatatttcgcttattcacgtagatagaattatttaa

SEQ ID NO: 230 - DNA, 1083 bp, African swine fever virus; identical to SEQ ID NO: 229

SEQ ID NO: 231 - DNA, 1170 bp, African swine fever virus
atgataataatccttatttttttaatatatttaaaactggtcttaaataataatattattatatggcatcctactttaaatgaaacaatttttttaaatgttacaactatagggaatattagtggtctattttggaatacatattataataataatcgtagtgaacttactaagtgcggaatatataataattattgttcatgtgatagtcttaatacatcattgtataatataaataattgtagtttagttatttttccaaataatacaaaaatatttaatacaatatatgaattaatatattcaggtaatcaaactaattatacaataaaattgctagaatctactactcctccaaatattacatataattgtactacatctgtaataacatgtaaaaaaaataatgggacaaatactaatatatttttaactataaataatacgattgttaattctactaatgaagatgttatttactactattggaatagaagtgaatttaataactttacagctacatgtatgattaataatacaattaattcatcaaatatatcagaaattatagactgtactaatacattattattaaacagttatttagacttttttcaagtagccggttatatgttttatatgataatttttattgtaactggaataacagtaagtatttttatttcaatcataacttttttatctttacgaaaaagaaaaaaacatgttgaagaaatagaaagtctaccacctgaatctaatgaagaagaatgtcagcatgatgatgacaccacttccatacatgaaccatcccccagagaaccattacttcctaaaccttacagtcgttatcagtataatacacctatttactacatgcgtccctcaacacaaccactcaacccatttcccctacctaaaccatgcccaccacctaaaccgtgtccgccacccaaaccatgtcctccacccaaaccatgcccgccacccaaaccatgcccgccacctaaaccatgtcctccacctgaatcatgttcttcacctgaatcatgttcttcacctgaatcatattctccacccaaaccactacctagtatcccgctgctacccaatatcccgccattatctacacaaaatatttcgcttattcatgtagatagaattatttaa

SEQ ID NO: 232 - DNA, 1221 bp, African swine fever virus
atgataatgttcataacacttatttttttaagttatattaatatagttttaagtaataattattgggctagacttaacgaaacaataactttaaatagtaacattactaatgatactaataatgaattaggtatattttggaattcttataataatacttattataataatacatttaataatattgctatatgtggaaaaaaaggtattttttgtgaatgtaatattaattataatacatctatatctaatacatctatatctaatacatcaatatataatgtaacaaataattgtagtttaacgatttttctttatgatgataatatatttaaaacatatcaattagtatatcagaattacaaaattaattatacaataaacttattattacctgttacttctccaaatattacatataattgtactaactctttaataacatgtgaaaaaaatgatgggacaaacactaatatgtttttatctataaataatataaccattaatcatactaatcaagatattcttacttactattggaataatagtgagtttaataattttacagctacatgtatgattaataatacacttaattcagcaaataccacaaaagttatcaattgcactaatccattattaaattcttaccaaaattattttcttgaaaatattcatacattattttatatcataatttttattgtgagtggattaatagcaagtatttttatttcaattataacttttttatctttacgaaaaagaaaaaaacatgttgaagaaatagaaagtccaccacctgaatctaatgaagaagaacaatgtcagcatgatgacaccacttccatacatgaaccatctcccagagaaccattacttcctaaaccttacagtcgttatcaatataatacacctatttactacatgcgtccctcaacacaaccactcaacccatttcccctacctaacccatgtcctccacctaaaccgtgtcctccacccaaaccatgcccgccacctaaaccatgtcccccacccaaaccatgcccgccacctaaaccgtgtcctccacccaaaccatgtccaccacctaaaccatgttcttcacctgaatcatattctccacccaaaccactacctagtatcccgctactacccaatatcccaccattatctacacaaaatatttcacttattcatgtagatagaattatttaa

SEQ ID NO: 233 - DNA, 1122 bp, African swine fever virus
atgataataatagttatttttttaatgtgtttaaaaatagttttaaacaatattataatatggagtactttaaatcaaactgtatttttaaataatatttttacaattaatgatacatatggtggtctattttggaatacatattatgataataatcgtagtaattttacttattgtggaatagcaggaaattattgttcatgttgtggtcataacatatcattgtataatacaacaaataattgtagtttaattatttttcctaacaatacagaaatatttaatagaacatatgaattagtatatttggacaaaaaaattaattatacagtaaaactattaaaatctgttgattccccaactattacatataattgtactaattctttaataacatgtaaaaataataatgggacaaatgttaatatatatttaattattaataatacaattagttcattgaatgaaacagaaaatataaattgtactaatccaatattaaaatatcaaaattatttatccacattattttatatcataatttttattgtgagtggattaataataggtatttttatttcaatcatatctgtattatctatacgaagaaaaagaaaaaaacatgttgaagaaatagaaagtccaccaccctctgaatctaatgaagaagatatttctcacgatgacaccacttccatacatgaaccatctcccagagaaccattacttcctaagccttacagtcgttatcagtataatacacctatttactacatgcgtccctcaacacaaccactcaacccatttcccctacctaaaccatgcccgccacctaaaccatgtcctccacccaagccatgcccgccacccaaaccatgtcctccacctaaaccgtgttctccacccaaaccgtgtcgtccacctaaaccatgtcctccacctaaaccatgtcctccacctaaaccatgtcctccacctaaaccatgtcctccatccaaaccatgtccttcacctgaatcctattctccacccaaaccactacctagtatcccgttactacccaatatcccgccattatctacacaaaatatttcgcttattcatgtagatagaattatttaa

SEQ ID NO: 234 - DNA, 1209 bp, African swine fever virus
atgataataatagttatttttttaatgtgtttaaaaatagttttaaacaatattataatatggagtactttaaatcaaactgtatttttaaataatatttttacaattaatgatacatatggtggtctattttggaatacatattatgataataatcgtagtaattttacttattgtggaatagcaggaaattattgttcatgttgtggtcataacatatcattgtataatacaacaaataattgtagtttaattatttttcctaacaatacagaaatatttaatagaacatatgaattagtatatttggacaaaaaaattaattatacagtaaaactattaaaatctgttgattccccaactattacatataattgtactaattctttaataacatgtaaaaataataatgggacaaatgttaatatatatttaattattaacaatacaattgttaatgatactaatggagatatccttaattattattggaatggtaataataattttacagctacatgtatgattaataatacaattagttcattgaatgaaacagaaaatataaattgtactaatccaatattaaaatatcaaaattatttatccacattattttatatcataatttttattgtgagtggattaataataggtatttttatttcaatcatatctgtattatctatacgaagaaaaagaaaaaaacatgttgaagaaatagaaagtccaccaccctctgaatctaatgaagaagatatttctcacgatgacaccacttccatacatgaaccatctcccagagaaccattacttcctaagccttacagtcgttatcagtataatacacctatttactacatgcgtccctcaacacaaccactcaacccatttcccctacctaaaccatgcccgccacctaaaccatgtcctccacccaagccatgcccgccacccaaaccatgtcctccacctaaaccgtgttctccacccaaaccgtgtcgtccacctaaaccatgtcctccacctaaaccatgtcctccacctaaaccatgtcctccacctaaaccatgtcctccatccaaaccatgtccttcacctgaatcctattctccacccaaaccactacctagtatcccgttactacccaatatcccgccattatctacacaaaatatttcgcttattcatgtagatagaattatttaa

SEQ ID NO: 235 - DNA, 1149 bp, African swine fever virus
atgataataaaggttatttttttagttttctttaaaatggttttaagtaatattataatatggagtactttaaataacacaatatatttaaatattaattattcagatagtaatttatatactggtctattttggaataaatattataataatactcgtaataattttactacttgtgtaacatcaaatagtagctattgtacatgtaaaggccataacacatcattgtataatataacaaataattgtagtttaatcatttttccaaataatacaaaaatatttaatacaacatatgaattagtatatttaaataataaaattaattatacaatacaaatggtacaacctgttgatcctccaattattatatataatgatactaataattctttaataacatgtgaaaaaaataatggaacgaatactgaaatctatttatatttaaatgatacatttattaataatactaatgaaaatagtattaaatactattggaattgtagcgaattaaaccataatattacaactacatgtattattaataatacacttaattcggcaaataccacaaaagttataaattgcactaatctattgttaaaatctgaccaaaattattttcttaaaaatattcatacattattttatatcataatttttattgtgggtggaacattaataagcattattatatcaatcataacttttttatctttacgaaaaagaaaaaaacatgttgaagaaatagaaagtccaccaccctctgaatctaatgaagaagaacattgtcagcatgatgataccacttccatacatgaaccatctcccagagaaccattacttcctaagccttacagtcgttatcagtataatacacctatttactacatgcgtccctcaacacaaccactcaacccatttcccctacctaaaccatgtcctccacctaaaccgtgtcctccacccaaaccatgtcctccacctaaaccgtgtcctccacctaaaccgtgtcccccacctaaaccatgtccttcacctgaatcccattctccacccaaaccactacctagtatcccgctgctacccaatattcccccattatctacacaaaatatttcgcttattcatgtagatagaattatttaa

SEQ ID NO: 236 - DNA, 1128 bp, African swine fever virus
atgataataatacttatttttttaataattcctaacatagttttaagtattgattattgggttagttttaataaaacaataattttagatagtaatattactaatgataataatgatataaatggagtatcatggaatttccttaataattctcttaatacactagctacatgtggaaaagcaggtaacttttgtgaatgctctaattatagtacatcactatataatatagcacataattgtagcttaactattttccctcataatgatgtatttggtacaccatatcaagtagtatggaatcaaataattaattatacaataaaattattaacacctgttactcccccaaatattacatataattgtactaattttttaataacatgtaaaaaaaataatggaacaaatactattatatatttcaatataaatgatactaatgttaaatatactaatgaaagtatacttgaatataactggaataatagtaactttaacaattttacagctacatgtataattaataatacaattaattcatcaaatgatacacaaactatagactgcatcaatacattattaagcagttatttagacttttttcaagtagccagttatatgttttatatgataatttttattgcaactggaataatagcaagtatttttatatcaatcataacttttttatctttacgaaaaagaaaaaaacatgttgaagaaatagaaagtccatcgccatctgaatctaatgaagaagaacaatgtcagcatgatgacaccacttccatacatgaaccgtctcccagagaaccattacttcctaagccttacagtcgatatcagtataatacacctatttactatatgcgtcccttaacacaaccactcaatccatctcccctacccaaactgtgtcctccacccaaaccgtgtcctccacccaaaccgtgtcctccacccaaaccgtgtcctccacccaaaccgtgtccttcatctgaatcatgttctccacctgaatcatattctctacccaaaccactacctaatatcccgctactacccaatatcccgccattatctacacaaaatatctcgcttattcacgtagatagaattatttaa

SEQ ID NO: 237 - DNA, 1158 bp, African swine fever virus
atgataataaagcttatttttttaatatcatttaaaatgattttaggtattgattattgggttagtcttaataatacaataattttagatagtaacattactattaataatattactaatactactaataatcctacattaaatggtatattttggaatatttataataatagttataataatacttttaatttacttaccacatgtgggaatacatataatatatgttcttgttctaataattataatacaatattatttaattataataccacaaataattgtagtttaattatttctcctcatgatgaaaaaatttttgatacgatgtttcagataatatatttaactaataaaattaattatacaatacgatggttacaacctgttgatcccccaaatatttcatttaatgatagtaattctttaataaaatgtgaaaaaaataatggaacaaatactgaaatctatttatatttaaatgatacatttattaataatactaatgaaaatgatcttaaatattattggaattgtagtgagttaaactataatattacagctacatgtattattaataatacacttaattcggcaaataccacaaaagttataaattgcactaatctattattaaaatctgaccaaaattattttcttaaaaacattcatacattattttatatcataatttttattgtaactgggataataataagtatttttatagcaatcataacttttttatctttacgaaaaagaaaaaaacatgttgaagaaatagaaagtccatcgcctgaatctaatgaagaggaagaacaacaacatcatcatgacactacttccatacatgaaccgtctcccagagaaccattacttcctaagccttacagtcgttatcagtataatacacctatttattacatgcgtccctcaacacaacaactatttaaatcatattctttacccaaaccatgtcccccacctaaaccatgtcctccacctaaaccatgtcccccacccaaaccatgtccctcaccagaatcatatccctcacctgaaccatatcctctactacctaatatcccactaccacccaatatcccgccattatctacacaaaatatttcgcttattcacgtagatagaattatttaa

SEQ ID NO: 238 - DNA, 1143 bp, African swine fever virus
atgataataaaacttatttttttaatttgttttaaaatagttttaagtattgataataaaacaaaatttaatgaaacattaattttagataatattgattattgggtaaagtttaatgatacaataactttagatagtaatattactagtgaaatagaagctgtatcttggaatttttataataatacttttaatctacttgctacatgtggaaaagcaagtaacttttgtagttgttctaattatagtacatcattttttaatattacaaataattgtagtttaactatttttttaatagacgagacgttatttaatacaacatatcaaatagtatattcaactaatataattaattataaaataaacttattaatacctgttactcctccaattatttcatataattgtactaattgttctataaattgtaaaaaaagtaatggtacaaatactaatatttttttatctataaatgatacaattgttacatatactaatgaaagtattcttaattatgattataactgtagtattttaaataataattttacagttacatgtataattaataatacaattagtacatcaaatactacagaatttataaactgcactaatatattattaagtagttatctagatttttttcaagtaactagttatatattttatatgataatttttattgtaactggaataacagtaagtattcttatatccatcataactttcttatttatacgaaaaagaaaacatgttgaagaaatagaaagtccaccgcctgaatctaatgaagaagaacaacaatgtcatcatgacaccacttctatacatgaaccgtctcccagagaaccattacttcctaagccttacagtcgttatcaatataatacacctatttactacatgcgtccctcaacacaacaactatttaaatcatattctttacccaaaccatgtcccccacctaaaccatgtcctccacccaaaccatgtcccccacccaaaccatgtcctccacccaaaccatgtcctccatccaaaccatatccttcactacctagtatcccgctaccacccgatatcccgccattatctacacaaaatatttcgcttattcacgtagatagaattatttaa

SEQ ID NO: 239 - DNA, 1179 bp, African swine fever virus
atgataataaaacttatttttttaatttgttttaaaatagttttaagtattgataataaaacaaaatttaatgaaacattaattttagataatattgattattgggtaaagtttaatgatacaataactttagatagtaatattactagtgaaatagaagctgtatcttggaatttttataataatacttttaatctacttgctatatgtggaaaagcaagtaacttttgtagttgttctaattatagtacatcattttttaatattacaaataattgtagtttaactatttttttaatagacgagacgttatttaatacaacatatcaaatagtatattcaactaatataattaattataaaataaacttattaatacctgttactcctccaattatttcatataattgtgctaattgttctataaattgtaaaaaaagtaatggtacaaatactaatatttttttatctataaatgatacaattgttacatatactaatgaaagtattcttaattatgattataactgtagtattttaaataataattttacagttacatgtataattaataatacaattagcacatcaaatactacagaatttataaactgcactaatatattattaagtagttatctagatttttttcaagtaactagttatatattttatatgataatttttattgtaactggaataacagtaagtattcttatatccatcataactttcttatttatacgaaaaagaaaacatgttgaagaaatagaaagtccaccgcctgaatctaatgaagaagaacaacaatgtcatcatgacaccacttctatacatgaaccgtctcccagagaaccattacttcctaagccttacagtcgttatcaatataatacacctatttactacatgcgtccctcaacacaacaactatttaaatcatattctttacccaaaccatgtcccccacctaaaccatgtcctccacctaaaccatgtcccccacctaaaccatgtcctccatccaaaccatgtcctccacctgaaccatattctccacccaaaccatgtcctccacccaaaccatatccttcactacctagtatcccgctaccacccgatatcccgccattatctacacaaaatatttcgcttattcacgtagatagaattatttaa

SEQ ID NO: 240 - DNA, 1146 bp, African swine fever virus
atgataattaaacttatttttttaatttgtttcaaaatagttttaagtattaattattgggttaggtataatgatacagtaactttaaatagtaatatcaatagcgagacagaaggtatattttggaatttttataataatacttttaatactattgccacatgtggaaaaaaaaataatgtttgtgaatgttctaattatgataaatccttatataatataacaaataattgtagtttaacaatttttccaaataatacaaaaatatttaatacaacatatcaattagtatattcacgcaatagaattaattacacaataaatttgttattacctgttacatctccaattattacatataattgtactcaatctttaataacatgtgaaaaaactaatggaacaaatattcatttattcttaaatttaaatgatacaattaatgaatatactaacaaaagttttctcaattactattggaatagtagtgagttaaataatatttttcttgctacatgtattattaataatacacttaattcagcaaatactacaaaagttatcaattgcactaacccattattaaaatcttaccaaaattattttcttgaaaatattcatacattattttatatgataatttttattgtatctggaataacaataagtatttttatatcaatcataacttttttatctttacgaaaaagaaaaaaacatgttgaagaaatagagagtccaccacctgaatctaatgaagaagaacaatgtcagcatgatgacaccacttctatacatgaaccatctcccagagaaccattacttcctaaaccttacagtcgttatcagtataatacacctatttactacatgcgtccctcaacacaaccactcaacccatttcccctacctaaaccatgcccgccacctaaaccgtgtcctccacccaaaccatgcccgccacccaaaccatgcccgccacctaaaccatgtcctccacccaaaccatgcccgccacctaaaccatgtcctccacctgaatcatattctccacccaaaccactacctagtatcccgctgctacccaatatcccgccattatctacacaaaatatttcgcttattcatgtagatagaattatttaa

SEQ ID NO: 241 - DNA, 1182 bp, African swine fever virus
atgatgataattatacttatttttttaatatgtttaaaaatagttttaaataataatattataatatggcatactttaaatgaaacaatttttttaaataatagtattacatttacggataatcttggtggtctattttggaatacatattataataataatcatagtatccttactagttgcggaatatatcaaaattattgttcatgcactaataattatagcacatcattatataatataacaaataattgtggtttaataatttttccaaataatacaaaaagatttaacacaacgtatgaattagtatatttaaataatagtactaaatatacaataaacttattagtacctgttactcctccaaatattacatataattgtactacatctgtaataacatgtaaaaaaaatgatggcacaaatactaatatgtttttaaaaataaatgatacaatcgttaaaagtagtaatgaaagtgtttttgaatactcttggaatagtactgatttaaataacaattttacggctatatgtataattaataatacaattaattcatcaaatgacacacaaactatagaatgtcccaatatattattaaacaattatttagacttttttcaagtaactagttacatgttttatatgataatttttattgtaactggaataatagcaagtatttttatttcaatcataacttttttatctttacgaaaaagaaaaaaacatgttgaagaaatagaaagtccaccaccctctgaatctaatgaagaagatatttctcacgatgacaccacttccatacatgaaccatctcccagagaaccattacttcctaagccttacagtcgttatcagtataatacacctatttactacatgcgtccctcaacacaaccactcaacccatttcccctacctaaaccatgcccgccacctaaaccatgcccgccacccaaaccatgtcctccacctaaaccgtgtcctccacccaaaccgtgtcgtccacctaaaccatgtcctccacctaaaccatgcccaccacctaaaccatgtccttcacctgaaacatattctccacccaaaccactacctaatatcccgctgctacccaatatcccgccattatctacacaaaatatttcgcttattcacgtagataggattatttaa

SEQ ID NO: 242 - protein, 360 aa, African swine fever virus; identical to SEQ ID NO: 24

SEQ ID NO: 243 - protein, 406 aa, African swine fever virus
MIMFITLIFLSYINIVLSNNYWARLNETITLNSNITNDTNNELGIFWNSYNNTYYNNTFNNIAICGKKGIFCECNINYNTSISNTSISNTSIYNVTNNCSLTIFLYDDNIFKTYQLVYQNYKINYTINLLLPVTSPNITYNCTNSLITCEKNDGTNTNMFLSINNITINHTNQDILTYYWNNSEFNNFTATCMINNTLNSANTTKVINCTNPLLNSYQNYFLENIHTLFYIIIFIVSGLIASIFISIITFLSLRKRKKHVEEIESPPPESNEEEQCQHDDTTSIHEPSPREPLLPKPYSRYQYNTPIYYMRPSTQPLNPFPLPNPCPPPKPCPPPKPCPPPKPCPPPKPCPPPKPCPPPKPCPPPKPCSSPESYSPPKPLPSIPLLPNIPPLSTQNISLIHVDRII

SEQ ID NO: 244 - protein, 373 aa, African swine fever virus
MIIIVIFLMCLKIVLNNIIIWSTLNQTVFLNNIFTINDTYGGLFWNTYYDNNRSNFTYCGIAGNYCSCCGHNISLYNTTNNCSLIIFPNNTEIFNRTYELVYLDKKINYTVKLLKSVDSPTITYNCTNSLITCKNNNGTNVNIYLIINNTISSLNETENINCTNPILKYQNYLSTLFYIIIFIVSGLIIGIFISIISVLSIRRKRKKHVEEIESPPPSESNEEDISHDDTTSIHEPSPREPLLPKPYSRYQYNTPIYYMRPSTQPLNPFPLPKPCPPPKPCPPPKPCPPPKPCPPPKPCSPPKPCRPPKPCPPPKPCPPPKPCPPPKPCPPSKPCPSPESYSPPKPLPSIPLLPNIPPLSTQNISLIHVDRII

SEQ ID NO: 245 - protein, 385 aa, African swine fever virus; identical to SEQ ID NO: 27

SEQ ID NO: 246 - protein, 380 aa, African swine fever virus
MIIKLIFLICFKIVLSIDNKTKFNETLILDNIDYWVKFNDTITLDSNITSEIEAVSWNFYNNTFNLLATCGKASNFCSCSNYSTSFFNITNNCSLTIFLIDETLFNTTYQIVYSTNIINYKINLLIPVTPPIISYNCTNCSINCKKSNGTNTNIFLSINDTIVTYTNESILNYDYNCSILNNNFTVTCIINNTISTSNTTEFINCTNILLSSYLDFFQVTSYIFYMIIFIVTGITVSILISIITFLFIRKRKHVEEIESPPPESNEEEQQCHHDTTSIHEPSPREPLLPKPYSRYQYNTPIYYMRPSTQQLFKSYSLPKPCPPPKPCPPPKPCPPPKPCPPPKPCPPSKPYPSLPSIPLPPDIPPLSTQNISLIHVDRII

SEQ ID NO: 247 - DNA, 414 bp, African swine fever virus
atgctggtgattatcctgggaattattggcctgctggccagtagcaacctggtttcatcctccacttctactcgggtaggtggacatcttcctctaacatttgaacccccagagaatgaacttggatactggtgcacttatgtggaaagctgtcggttctgctgggattgtgaagatgggatttgtaccagcagggtttggggaaacaactccacaagtattattgagaatgactatgtaaaatattgtgaggtttcccgctggggtgacctatgtagatatgatgtggaggagcacatttaccatagcatgaactgttctgacccaaagccctggaatccttataaaattgcaaggaaggagtggaaaaagaatgaacatcccagaaaggatttgaaaaaagatgaattctag

SEQ ID NO: 248 - DNA, 414 bp, African swine fever virus; identical to SEQ ID NO: 247

SEQ ID NO: 249 - DNA, 414 bp, African swine fever virus
atgctggtgattatcctgggagttattggcctgctggccagtagcaacttggtttcatcctccacttctactcggataggtggacatcttcctctaacatttgatcctccagagaatgaacttggatactggtgcacttatgtggaaagctgtcggttctgctgggattgtgaagatggggtctgtactagcaggatctgggggaacaactccacaagtattgttgagaacagctatgtaaaatattgtgaagtttcccgctggggtgaccagtgtagatatgatgtggaggagcgcatttaccataccatgaactgttctgacccaaaaccctggaatccttataaaattacaagggtggagtggaaaaagaatgaacatttcagaaaggatttgaaaaaagatgaattctag

SEQ ID NO: 250 - DNA, 414 bp, African swine fever virus
atgctggtgattatcctgggagttattggcctgctggccagtagcaacttggtttcatcctccacttctactcgggtaggtggacatcttcctctaacatttgatcctccagagaatgaacttggatactggtgcacttatgtggaaagctgtcggttctgctgggattgtgaagatggggtctgtactagcaggatctgggggaacaactccacaagtattgttgagaacagctatataaaatattgtgaagtttcccgctggggtgaccagtgtagatatgatgtggaggagcacatttactataccatgaactgttctgacccaaagccctggaatccttataaaattgcaaggaaggagtggaaaaagaatgaacatttcagaaaggatttgaaaaaagatgaattctag

SEQ ID NO: 251 - DNA, 378 bp, African swine fever virus
atgctggtgattatcctgggagttattggcctgctggccagtagcaacttggtttcatcctccacttctactcgggtaggtggacatcttcctctaacatttgatcctccagagaatgaacttggatactggtacacttatgtggaaagttgtcggttctgctgggattgtgaagatggggtctgtactagcagggtttggggaaacaactccacaagtattgttgagaatgactatgtaaaatattgtgaggtttctcgctggggtgaccaatgtagatatgatgtggaggagcacatttactataccatgaactgttctgacccaaagccctggaatccttataaaattgcaaaggaaggagtggaaaaaggatga

SEQ ID NO: 252 - DNA, 414 bp, African swine fever virus
atgctggtgattatcctgggagttattggcctgctggccagtagcaacttggtttcatcctccacttctactcgggtaggtggacatcttcctctaacagaggatcctccagagaatgaacttggatactggtgcacttatgtggaaagctgtcggttctgctgggattgtgaagatgggatttgtactagcagggtttggggaaacaactccacaagtattgttgagaacgactatgtaaaatattgtgaggtttctcgctggggtgaccaatgtagatatgatgtagaggagcacatttactataccatgaactgttctgacccaaagccctggaatccttataaaattgcaaggaaggagtggaaaaaggatgaacattccagaaagaatttgaaaaaagatgaattctag

SEQ ID NO: 253 - DNA, 414 bp, African swine fever virus
atgctggtgattatcctgggaattattggcctgctggccagtagcaacctggtttcatcctccacttctactcgggtaggtggacatcttcctctaacatttgatcccccagagaatgaacttggatactggtgcacttatgtggaaagctgtcggttctgctgggattgtgaagatgggatttgtaccagcagggtttggggaaacaactccacaagtattgttgagaatgactatgtaaaatattgtgaggtttcccgctggggaaacctatgtagatatgatgtggaggagcacatttactatagcatgaactgttctgacccaaagccctggaatccttataaaattgcaaggaaggagtggaaaaagaatgaatatctcagaaaggatttgaaaaaagatgaattctag

SEQ ID NO: 254 - DNA, 105 bp, African swine fever virus
atgaactgttctgacccaaagccctggaatccttataaaattgcaaggaaggagtggaaaaagaatgaacatcccagaaaggatttgaaaaaagatgaattctag

SEQ ID NO: 255 - DNA, 414 bp, African swine fever virus
atgctggtgattatcctgggaattattggcctgctggccagtagcaacctggtttcatcctccacttctactcgggtaggtggacatcttcctctaacatttgatcccccagagaatgaacttggatactggtgcacttatgtggaaagctgtcggttctgctgggattgtgaagatgggatttgtaccagcagggtttggggaaacaactccacaagtattgttgagaatgactatgtaaaatattgtgaggtttcccgctggggtaacctatgtagatatgatgtggaggagcacatttaccatagcatgaactgctctgacccaaagccctggaatccttataaaattgcaaggaaggagtggaaaaagaatgaacatcccagaaaggatttgaaaaaagatgaattctag

SEQ ID NO: 256 - DNA, 414 bp, African swine fever virus
atgctggtgattatcctgggaattattggcctgctggccagtagcaacctggtttcatcctccacttctactcgggtaggtggacatcttcctctaacatttgatccccccgagaatgaacttggatactggtgcacttatgtggaaagctgtcggttctgctgggattgtgaagatgggatttgtaccagcagggtttggggaaacaactccacaagtattgttgagaatgactatgtaaaatattgtgaggtttcccgctggggtaacctatgtagatatgatgtggaggagcacatttaccatagcatgaactgctctgacccaaagccctggaatccttataaaattgcaaggaaggagtggaaaaagaatgaacatcccagaaaggatttgaaaaaagatgaattctag

SEQ ID NO: 257 - DNA, 1587 bp, African swine fever virus
atgttctccctacaggacctctgtcggaagaacactttcttccttccaaatgattttagcaagcatactctacaacggctggggttgtattggaaagagcatggatccgtccatcgaatagaaaaggacagcataatgatacagaatgaattggttctttctatcaatgatgctttacagcttgcaggagaggagggggacacagatgtggtacagctcttgttactatgggagggaaatctgcattatgccatcataggagctttgaagactgagaattataatctagtatgtgagtaccatagccaaattcaggactggcatattctcctacccttgattcaagatccagaaacattcgaaaaatgtcatgatttaagccttggatgtgaccttatatgccttctccaacatgctgtaaaatgtgatatgctttctattcttgttaaatataaggaggatttactaaatgtaaggattaggcatcgtacccaatccctgtttgttttggcatgcgaaaatcggagatttgagattattgaatggataggtcaaaatctgtcaattcctgaacctgaggccatttttagcattgctattgttacaaaagatgtagaactgttttccttaggatataaaattatttttgattacatgcaaagacaaggaatttttcaattaaccaatgtagttcgcatgcttctgctaaatcgtcatattggtatggcaatagaaaaaggacttttaccttttattctggaaactttaaaatatggtggtagtgtaaaaagagctttatcttatgcagtaatagataataaaagaaaaattatagactatcttgtacgccatgaaaatataccccgaggaactattgaaagacttttgcatctagctgtgaaaaaacaatcttccaggaaaactttgaacttgttgctatcttacataaattacaaggtgaaaaatgttaaaaagctggtagagcatgtagtagatcacaaatccactcttgtgttaaaaattttattggaaaaaaaggaaaatctagtggatgctgttttaacaagacttgtaaaacattctacatatttccaggtgagagaatttatccaggagttttccatcagcccagaaaaattcattaaaatagctgtgcgggaaaagaaaaatgtgttaatcgaggctatttctgaagatatttgggaaaatcccacagaaagaattacttatctcaaacagatagtgcacaccataaaatatgaaagtggaaggcggtttttgatagacatcattcacagcatttaccaaagttactcactaaaacacgaagatattcttaaactggcaacattttatgtcaaacacaatgcaatcacccattttaaagatctctgcaaatatctttggctgaacagaggaacagaaagtaagaaactgtttttagagtgcttggaaattgctgatgagaaggagtttcctgatattaaaaatattgtgagtgaatatattaactacttgtttactgcaggagctattaccaaggaagaaatcatgcaagcttatgctttagagtatgacatgtattaa

SEQ ID NO: 258 - DNA, 1587 bp, African swine fever virus
atgttctccctacaggacctctgtcggaagaacactttcttccttccaaatgattttagcaagcatactctacaacggctggggttgtattggaaagagcatggatccgtccatcgaatagaaaaggacagcataatgatacagaatgaattggttctttctatcaatgatgctttacagcttgcaggagaggagggggacacagatgtggtacagctcttgttactatgggagggaaatctgcattatgccatcataggagctttgaagactgagaattataatctagtatgtgagtaccatagccaaattcaggactggcatattctcctacccttgattcaagatccagaaacattcgaaaaatgtcatgatttaagccttggatgtgaccttatatgccttctccaacatgctgtaaaatgtgatatgctttctattcttgttaaatataaggaggatttactaaatgtaaggattaggcatcgtacccaatccctgtttgttttggcatgcgaaaatcggagatttgagattattgaatggataggtcaaaatctgtcaattcctgaacctgaggccatttttagcattgctattgttacaaaagatgtagaactgttttccttaggatataaaattatttttgattacatgcaaagacaaggaatttttcaattaaccaatgtagttcgcatgcttctgctaaatcgtcatattggtatggcaatagaaaaaggacttttaccttttattctggaaactttaaaatatggtggtagtgtaaaaagagctttatcttatgcagtaatagataataaaagaaaaattatagactatcttgtacgccatgaaaatataccccgaggaactattgaaagacttttgcatctagctgtgaaaaaacaatcttccaggaaaactttgaacttgttgctatcttacataaattacaaggtgaaaaatgttaaaaagctggtagagcatgtagtagatcacaaatccactcttgtgttaaaaattttattggaaaaaaaggaaaatctagtggatgctgttttaacaagacttgtaaaacattctacatatttccaggtgagagaatttatccaggagttttccatcagcccagaaaaattcattaaaatagctgtgcgggaaaagaaaaatgtgttaatcgaggctatttctgaagatatttgggaaaatcccacagaaagaattacttatctcaaacagatagtgcacaccataaaatatgaaagtggaaggcggtttttgatagacatcattcacagcatttaccaaagttactcactaaaacacgaagatattcttaaactggcaacattttatgtcaaacacaatgcaatcacccattttaaagatctctgcaaatatctttggctgaacagaggaacagaaagtaagaaactgtttttagagtgcttggaaattgctgatgagaaggagtttcctgatattaaaagtattgtgagtgaatatattaactacttgtttactgcaggagctattaccaaggaagaaatcatgcaagcttatgctttagagtatgacatgtattaa

SEQ ID NO: 259 - DNA, 1578 bp, African swine fever virus
atgttctccctacaggacctctgtcggaagaacattttcttccttccaaatgattttagcaagcataccctacaatggctgggattatattggaaagagcatggatccgtccatcgagcagaaaaagacagcataatgatacagaatgaattggttctttctatcaatgatgctttacagcttgcaggagaggagggggacacagatgtagtacagctcttgttattatgggagggaaatctgcattatgccatcataggagccttgaagactgaaaaatataacctaatatgtgagtatcatagccaaattcaggactggcatattctcctacccatgattcaagatccagaaacattcgaaaaatgtcatgatttaagccttggatgtgactttatttgccttctccaacatgctgtaaaatacaacatgctttctattcttgtcaaatataaggaggatctactaaatgcaaggattaggcatcgtatccaatccctgtttgttttggcatgcgaaaatcggagaattgaaattattgattggataggccaaaatctgccaattcctgaacctgatgccatttttagcattgctgttgctacaagagatttagaactgttttccttagggtacaagattatttttgattacatgcaaagacagggaatcattcaattaaccaatggagttcgcatggttgtgctaaatcgtcacattagcatggcaatagataatggtcttttaccttttgttctggaaactttaaaacatggtgggaatatacatagagccttatcttatgcagtaacacacaatagaagaaaaattctggattatcttattcgccagaaaaatatagcccctaatacaattgaaagacttttatatctggccgtgaaaaatcaatcttccaggaaaactttgaacttgttgctatcttacataaattacaaggtgaaaaatgttaaaaagctggtagagcatgtagtaaatgagaaatccactcttgtgttaaaaattttattagaaaaaaaggaaaatctagtggatgctgttttaacaagacttgtaaaacattctacatatttccaggtgagagaatttatccaggagttttccatcagcccagaaaaattcattaaaatagctgtgcgggaaaagaaaaatgtgttaatcgaggctatttctgaagatatttgggaaaatcccacagaaagaattacttatctcaaacagatagtgcacaccataaaatatgaaagtggaaggcgatttttggtagacatcattcacagcatttaccaaagttactcactaaaacacgaagatattcttaaactggcaacattttatgtcaaacacaatgcaatcacccattttaaagacctctgcaaatatctttggctgaacagaggaacagaaagtaagaaactgtttttagagtgtttagaaattgctgatgagaaggagtttcctgatattaaaagtattgtgagtgaatatattaactacttgtttactgcaggagctattaccaaggaagaaatcatgcaagcctatgatgctttagagtag

SEQ ID NO: 260 - DNA, 1578 bp, African swine fever virus; identical to SEQ ID NO: 259

SEQ ID NO: 261 - DNA, 1578 bp, African swine fever virus
atgttctccctacaggacctctgtcggaagaacactttcttccttccaaatgattttagcaatcataccctacaacggctggggttatattggaaagagcatggatccgtccatcgaatagaaaaggacagcataatgatacagaatgaattggttctttctatcaatgatgctttacatcttgcaggagaggaggggaacacagatgtagtacagctcttgttattatgggagggaaatctgcattatgccatcataggagccttgaagactgagaaatataacctaatatgtgagtaccatagccaaattcagaactggcatgttctcctccccttgattcaagatccagaaacattcgaaaaatgtcatgatttaagccttggatgtgactttatttgccttctccaacatgctgtaaaatgtgacatgctttctattcttgttaaatataaggaggatctactaaatgcaaggattaggcatcgtatccaatccctgtttgttttggcatgcgaaaatcggagatttgagattattgaatggataggtcaaaatctgccaattcctgaacctgaggccatttttagcattgctattgttacaaaagatatagaactgttttccttagcgtacaagcttatttttgattacatgcaaagacaaggaacttttcaattaaccaatatggtccgcatgcttctgctaaatcggtacattggtatggcaatagaaaaaggacttttaccctttattctggaaactttaaaatatggtggtagtgtaaatagagctttatcttatgcagtaatagataataaaagaaaaattatagactatcttgtacgccatgaaaatataccccgtggaactattgaaagacttttgcatctagctgtgaaaaaacaatcttccagaaaaactttgaacttgttgctatcttacataaattacaaggtgaaaaatgttaaaaagctggtagagcatgtagtagatcacaactctactcttgtgttaaaaattttattggaaaaaaaggaaaatctagtggatgctgttttaacaagacttgtaaaacattctacatatttccgggtaagagaatttatccaggagttttccatcagcccagaaaaattcattaaaatagctgtgcgggaaaagaaaaatgtgttaatcgaggctatttctgaagatatttgggaaaatcccacagaaagaattacttatctcaaacagatagtgcacaccataaaatatgaaagtggaaggcggtttttgatagacatcattcacagcatttaccaaagttattcactaaaacatgaagatatttttaaactggcaacattttatgtcaaacacaatgcaatcacccattttaaagatctctgcaaatatctttggctgaacagaggaacagaaagtaagaaactgtttttagagtgcttggaaattgctgatgagaaggagtttcctgatattaaaagcattgtgagtgaatatattaactacttgtttactgcaggagctattaccaaggaagaaatcatgcaagcctataatgctttagagtag

SEQ ID NO: 262 - DNA, 1557 bp, African swine fever virus
atgttctcccttcaggacctctgtcggaagaacattttcttccttccaaacgattttagcaagcataccctacaatggctggggttatattggaaagagcatggatccgtccatcgagcagaaaaagacagcataatgatacagaatgaattggttctttctatcaatgatgctttactgcttgcaggagaggagggggacacagatgtagtacagctcttgttgctatgggaaggaaatctgcattatgccatcataggagccttgaagactgagaaatatagcctagtatgtgagtaccatagccaaattcaggactggcatgttctcctccccttgattcaagatccagaaacattcgaaaaatgtcatgatttaagccttgaatgtgactttatatgccttctacaacatgctgtaaaatgtgacatgctttctattcttgttaaatataaagaggatctactaaatgtaaggattaggcatcgtatccaatccctgtttgttttggcatgcgaaaatcggagatttgagattattgaatggataggtcaaaatctgccaattcctgaacctgaggccatttttagcattgctattgttacaaaagatatagaactgttttccttaggatacaagcttatttttgattacatgcaaagacaaggaacttttcaattaaccaatatggttcgcatgcttctgctaaatcgttacattggtatggcaatagaaaaaggccttttaccctttatcgtggaaactttaaaatatggtggtagtgtaaatagagctttatcttatgcagtcatagataataaaagaaaaattatagactatcttgtacgtcatgaaaatataccccgtggaactattgaaagacttttgcatctagctgtgaaaaaacaatcttccagaaaaactttgaacttgttgctatcttacataaactacaaggtgaaaaatgttaaaaagctggtagagcatgtagtagatcacaactctacccttgtgttaaaaattttattggaaaaaaaggaaaatttagtggatgctgttttaacaagacttgtaaaacattctacatatttccgggtgagagaatttatccaggagttttccatcagcccagaaaaattcattaaaatagctgtgcgggaacagaaaaatgtgttaatcgaggctatttctgaagatatttgggaaaatcccacagaaagaattacttatctcaaacagatagtgcacaccataaaatatgaaagtggaaggcggtttttgatagacatcattcacagcatttaccaaagttactcactaaaacacgaagatattcttaaactggcaatattttatgtcaaatacaatgcaatcacccattttaaagatctctgcaaatatctttggctgaacagaggaacagaaagtaagaaactgtttttagagtgcttggaaattgctgatgagaaggagtttcctgatattaaaagcattgtgagtgaatatattaactacttgtttactgcaggagctattactaaggaagaaatcatataa

SEQ ID NO: 263 - DNA, 1581 bp, African swine fever virus
atgttctcccttcaggacctctgtcggaagaacactttcttccttccaaatgattttagcaagcataccctacaacggctggggttatattggaaagagcatggatccgtccatcgagcagaaaaagacagcataatgatacagaatgaattggttctttctatcaatgatgctttacagcttgcaggagaggagggggacacagatgtagtacagctcttattattatgggaaggaaatcttcattatgccatcataggagccttgaagactgagaaatataacctaatatatgagtaccatagccaaattcaggactggcatgttctcttacccatgattcaagatccagaaacattcgaaaaatgtcatgatttaagccttgcatgtgactttatatgccttctccaacatgctataaaatataacatgctttctatccttgtcaaatataaggaagacttactaaatgtaaggattaggtatcgtatccaatccctgtttgttttggcatgtgaaaaccggagaattgagattattgaatggataggtcaaaatctgccaattcctgaacctgatgccatttttagcattgctgttgctacaagagatttagaactgttttccttagggtacaagattatttttgattacatgcaaagacagggaatctttcaattaaccaatggagttcgcatggttgtgctaaatcgtcacattagcatggcaatagataatggtcttttaccctttgttctggaaactttaaaacatggtgggaatatacatagagccttatcttatgcagtaacacacaataaaagaaaaattctggattatcttattcgccagaaaaatatagcccctaatacaattgaaagacttttatatctggccgtgaaaaatcaatcatccagaaaaactttgaacttgttactatcttacataaattacaaggtgaaaaatgttaaaaagctggtagagcatgtagtaaatgagaaatccactcttgtgttaaaaattttattagaaaaaaaggaaaatctagtggatgctgttttaacaagacttgtaaaacattctacatatttccaggtgagagaatttatccaggagttttccatcagcccagaaaaattcattaaaatagctgtgcgggaaaagaaaaatgtgttgatcgaggctatttctgaagatatttgggaaaatcccacagaaagaattacttatctcaaacagatagtgcacaccataaaatatgaaagtggaaggcgatttttggtagacatcattcacagcatttaccaaagttactcactaaaacacgaagatattcttaaactggcaacattttatgtcaaacacaatgcaaccacccattttaaagatctctgcaaatatctttggctgaacagaggaacagaaagtaaaaaactctttttagagtgtttagaaattgctgatgagaaggagtttcctgatattaaaagcattgtgagtgaatatattaactacttgtttactgcaggagctattaccaaggaagaagcctatggatgctttagagtagccatgtattaa

SEQ ID NO: 264 - DNA, 1578 bp, African swine fever virus
atgttctccctacaggacctctgtcggaagaacattttcttccttccaaacgattttagcaagcataccctacaatggctggggttatattggaaagagcatggatccgtccatcgagcagaaaaagacagcataatgatacagaatgaattggttctttctatcaatgatgctttactgcttgcaggagaggagggggacatagatgtagtacagctcttgttattatgggagggtaatctgcattatgccatcataggagccttgaagactgagaaatataacctaatatgtgagtaccatagccaaattcaggactggcatattctcctacccatgattcaagatccagaaacattcgaaaaatgtcatgatttaagccttgcatgtgactttatatgccttctccaacatgctgtaaaatataacatgctttctatccttgtcaaatataaggaagacttactaaatgtaaggattaggtatcgtatccaatccctgtttgttttggcatgcgaaaaccggagaattgagattattcattggataggccaaaatctgccaattcctgaacctgatgccatttttagcattgctgttgctacaagagatttagaactgttttccttagggtacaagattatttttgattacatgcaaagacagggaatctttcaattatccaatggagttcgcatggttgtgctaaatcgtcacattagcatggcaatagataatggacttttaccctttgttctggaaactttaaaacatggtgggaatatacatagagccttatcttatgcagtaacacacaatagaagaaaaattctggattatcttattcgccagaaaaatatagcccctaatacaattgaaagacttttatatctggccgtgaaaaatcaatcttccaggaaaactttgaacttgttgctatcttacataaattacaaggtgaaaaatgttaaaaagctggtagagcatgtagtaaatgagaaatccactcttgtgttaaaaattttattggaaaaaaaggaaaatctagtggatgctgttttaacaagacttgtaaaacattctacatatttccaggtgagagaatttatccaggagttttccatcagcccagaaaaattcattaaaatagctgtgcgggaaaagaaaaatgtgttaatcgaggctatttctgaagatatttgggaaaatcccacagaaagaattacttatctcaaacagatagtgcacaccataaaatatgaaagtggaagacgatttttgatagagatcattcacagcatttaccaaagttactcactaaaacacgaagatattcttaaactggcaacattttatgtcaaacacaatgcaaccacccattttaaagatctctgcaaatatctttggctgaacagaggaacagaaagtaagaaactgtttttagagtgtttagaaattgctgatgagaaggagtttcctgatattaaaagtattgtgagtgaatatattaactacttgtttactgcaggaactattaccaaggaagaaatcatgcaagcctatgatgctttagagtag

SEQ ID NO: 265 - DNA, 1578 bp, African swine fever virus
atgttctcccttcaggacctctgtcggaagaacattttcttccttccaaatgattttagcaagcataccctacaatggctggggttatattggaaagagcatggatccgtccatcgagcagaaaaaaacagcatagtgatacagaatgaattggttctttctatcaatgatgctttacagcttgcaggggaagagggggacacagatgtagtacagctcttgttattatgggagggaaatctgcattatgccatcataggagccttgaagactgagaaatataatctaatatgtgagtaccatagccaaattcaggactggcatgttctcctccccttgattcaagatccagaaacattcgaaaaatgtcatgatttaagccttgcatgtgactttatatgccttctacaacatgctgtaaaatataacatgctttctatccttgtcaaatataaggaagacttactaaatgtaaggattaggtatcgtatccaatccctgtttgttttggcatgtgaaaaccggagaattgagattattgattggataggccaaaatctgccaattcctgaacctgatgccatttttagcattgctgttgctacaagagatttagaactgttttccttagggtacaagattatttttgattacatgcaaagacagggaatctttcaattatccaatggagttcgcatggttgtgctaaatcgtcacattagcatggcaatagataatggacttttaccctttgttctggaaactttaaaacatggtgggaatatacatagagccttatcttatgcagtaacacacaatagaagaaaaattctggattatcttattcgccagaaaaatatagcccctaatacaattgaaagacttttatatctggccgtgaaaaatcaatcttccaggaaaactttgaacttgttgctatcttacataaattacaaggtgaaaaatgttaaaaagctagtagagcatgtagtaaatgagaaatccactcttgtgttaaaaattttattggaaaaaaaggaaaatctagtggatgctgttttaacaagacttgtaaaacattctacatatttccaggtgagagaatttatccaggagttttccatcagcccagaaaaattcattaaaatagctgtgcgggaaaagaaaaatgtgttaatcgaggctatttctgaagatatttgggaaaatcccacagaaagaattacttatctcaaacagatagtgcacaccataaaatatgaaagtggaagacgatttttgatagacatcattcacagcatttaccaaagttactcactaaaacacgaagatattcttaaactggcaacattttatgtcaaatacaatgcaatcacccattttaaagatctctgcaaatatctttggctgaacagaggaacagaaagtaagaaactgtttttagagtgtttagaaattgctgatgagaaggagtttcctgatattaaaagcattgtgagtgaatatattaactacttgtttactgcaggagctattaccaaggaagaaatcatgcaagcctatgatgctttagagtag

SEQ ID NO: 266 - DNA, 618 bp, African swine fever virus
atgttggtgatcttcttgggaattcttggccttctggccagccaggtttcaagtcagctcgttggacaacttcgtccaacagaggagcctccagaggaagaactcgaatactggtgtgcctacatggaaagttgccaattttgctgggactgccaagacggcacttgtataaacaaaatagatgggtcagtaatttacaagaatgagtatgtgaaatcatgtctggtttcccgttggctggataaatgtatgtatgatttagataagggtatctaccataccatgaattgcaaccaggtcttagggctacctaatcaaccagctggacaacttcatccaacggataatcctccacaagaagaacttgaatactggtgcacttatacagaaaactgcaagttttgctggaattgtcaaaatggcctttgtgagggcaaattggagaatacaacaattcttgaaaatgagtatgtgcaatcatgtatagtttcccgctggctaaataaatgtatgtatgatctaggacaggggattcaccatgtaatggcctgttctgaaccaaagccctggaatccttacaaaatcttaaagagagagtggaaagaaaataatagttaa

SEQ ID NO: 267 - DNA, 618 bp, African swine fever virus; identical to SEQ ID NO: 266

SEQ ID NO: 268 - DNA, 372 bp, African swine fever virus
atgttggtgatcttcttgggactccttggcctgctggccaaccaggtcttagggctacctaatcaaccagctggacaacttcatccaacggataatcctccaaaagaagaacttggatactggtgcacttacacggaaagctgtaaattttgctggaactgtcaaaatggcctttgtgagggcaaattggagaatacaacaattcttgaaaatgagtatgtgcaatcatgtatagtttcccgctggctaaataaatgtatgtatgatctaggacaggggattgaccatgtaatggcctgttctgaaccaaagccctggaatccttacaaaatcttaaagagagagtggaaaaaaaatgtcagccaaaattaa

SEQ ID NO: 269 - DNA, 372 bp, African swine fever virus
atgttggtgatcttcttgggactccttggcctgctggccaaccaggtcttagggctacctaatcaaccagctggacaacttcatccaacggataatcctccaaaagaagaacttggatactggtgcacttacacggaaagctgtaaattttgctggaactgtcaaaatggcctttgtgagggcaaattggagaatacaacaattcttgaaaatgagtatgtgcaatcatgtatagtttcccgctggctaaataaatgtatgtatgatctaggacaggggattcaccatgtaatgccctgttctgaaccaaagccctggaatccttacaaaatcttaaagaaagagtggaaagaaaatgtcagccaaaattaa

SEQ ID NO: 270 - DNA, 363 bp, African swine fever virus
atgttggttatcttcttgggaattcttggccttctggccaaccaggtctcaagccagctcgttggacaacttcatccaacggaaaatccttcagagaatgaacttgaatactggtgcacttacatggaatgttgccagttttgctgggactgtcaagatggcctttgtgtgaataagttgggaaatacaacaattcttgaaaatgagtatgtgcatccatgtatagtttcccgctggctaaataaatgtatgtatgatctgggtcaaggcattgatcatgtaatggtctgttctcaaccaaagtactggaatccttataaaatcttaaagaaagagtggaaagaaaataacagccaaaattaa

SEQ ID NO: 271 - DNA, 363 bp, African swine fever virus
atgttagtaatcttcttgggaattcttggccttctggccaaccaggtctcaagccagctcgttggacaacttcatccaacggaaaatccttcagagaatgaacttgaatactggtgcacttacatggaatgttgccagttttgctgggactgtcaagatggcctttgtgtgaataagttgggaaatacaacaattcttgaaaatgagtatgtgcatccatgtatagtttcccgctggctaaataaatgcatgtatgatctgggtcaaggcattgatcatgtaatggtctgttctcaaccaaagtactggaatccttataaaatcttaaagaaagagtggaaagaaaataacagtcaaaattaa

SEQ ID NO: 272 - DNA, 366 bp, African swine fever virus
atgttagtaatcttcttgggaattcttggccttctggccaaccaggtctcaagccagctcgttggacaacttcatccaacggaaaatccttcagagaatgaacttgaatactggtgcacttacatggaatgttgccagttttgctgggactgtcaaaatggcctttgtgtgaataagttgggaaatacaacaattcttgaaaatgagtatgtgcatccatgtatagtttcccgctggctaaataaatgtatgtatgatctgggtcaaggcattgatcatgtaatggtctgttctcagccaaaatactggaatccttataaaatcttaaagaaagagtggaaagaaaataacagccaaaataaataa

SEQ ID NO: 273 - DNA, 357 bp, African swine fever virus
atgttagtcatcttcttgggaattcttggccttctggtcaaccaggtctcaagccagccagttggacaacttcatccaacggaaaatccttcagagaatgaacttgaatactggtgcacttacatggaatgttgccagttttgctgggactgtcaaaatggcctttgtgtgaataagttgggaaatacaacaattcttgaaaatgagtatgtgcatccatgtatagtttcccgctggctaaataaatgtatgtatgatctgggtcaaggcattgatcatgtaatggtctgttctcaaccaaaatactggaatccttataaaatcttaaagaaagagtggaaagaaaataatagttaa

SEQ ID NO: 274 - DNA, 366 bp, African swine fever virus
atgttagtaatctttttgggaattcttggccttctggccaaccaggtctcaagccagctcgttggacaacttcatccaacggaaaatccttcagagaatgaacttgaatactggtgcacttacatggaatgttgccagttttgctgggactgtcaaaatggcctttgtgtgaataagttgggaaatacaacaattcttgaaaatgagtatgtgcatccatgtatagtttcccgctggctaaataaatgtatgtacgatctgggtcaaggcattgatcatgtaatggtctgttctcaaccaaaatactggaatccttataaaatcttaaagaaagagtggaaagaaaataacagccaaaataaataa

SEQ ID NO: 275 - DNA, 366 bp, African swine fever virus
atgttagtaatcttcttgggaattcttggccttctggccaaccaggtctcaagccagctcgttggacaacttcatccaacggaaaatccttcagagaatgaacttgaatactggtgcacttacatggaatgttgccagttttgctgggactgtcaaaatggcctttgtgtgaataagttgggaaatacaacaattcttgaaaatgagtatgtgcatccatgtatagtttcccgctggctaaataaatgtatgtacgatctgggtcaaggcattgatcatgtaatggtctgttctcaaccaaaatactggaatccttataaaatcttaaagaaagagtggaaagaaaataacagccaaaataaataa

SEQ ID NO: 276 - DNA, 312 bp, African swine fever virus
atgaaggtttttctaggacttttactaggttattcaaccatcctcattcttacatatcaatcaccaacaacccagtggtgtttttatgaaatatcacttaaaatacttaatcatcatagcatggaaaaatggagggataagaattggtcaatcattataaggtattattgtttttaccttgtatttagctttgcatttgctggttgcgttgcatttgcgatctgcaaaaatctacgactgtgtacaaccatgaaattacttatgcttttgaatattttggttttgttatctcagccaattttgaataattga

SEQ ID NO: 277 - DNA, 360 bp, African swine fever virus
atgaaggtttttctaggacttttactaggttattcaaccatccttattcttacctatcaatcaccaacaaccccgtggtgtttttatgaaatatcacttaaaatacctaatcatcatagcatgaaatgttgttcgtatcctagactttatgaacatgaaatgttcatggaaaaatggagggataagaattggccaatcattataaggtattattgtttttaccttgtatttagctttgtatttgctggttgcgttgcatttgcgatctgcaaaaatctacgactgagtacaaccatgaaattacttatgcttttgagtattttggttttgttgttatctcagccaatcttgaataattga

SEQ ID NO: 278 - DNA, 360 bp, African swine fever virus; identical to SEQ ID NO: 277

SEQ ID NO: 279 - DNA, 507 bp, African swine fever virus
atgaaggtttttctaggacttttactaggtttttcaatcatcctcattcttacatatcaatcaccgacaactcagcatcctcctaaggaagagcttgcgtactggtgcacttatgcaaaatcttgtgacttctgctgggattgccaaaatgacacttgtataaataaggtaataaatgagtctatttcgataacttcgattgtaaactgtagagttactcgcgattcccaatcctgtttttatgaaatatcagttaaaatacctaatcatcatagcatggaatgttcatatcctagactttatgaacatgaaatgtttatggaaaaatggagggatgagtattggccaatcattataaaacagtgttgtttttaccttgtatttagcattgcatttgctggatgcgttgcatttgcgatctgcaaaaatctacgactgcgtacaactataaaactacttattcttttgagtattttggtttggttgtctcaaccagtcttgaattga

SEQ ID NO: 280 - DNA, 516 bp, African swine fever virus
atgaaggtttttctggcacttttactaggttatttaactatcctcattcttacatatcaaacaccaacaacccagcatcctcctaaggaggagcttccatattggtgtacgtatgtaaagaattgcgacctctgttgggattgtcaagatagcatctattggaacaaggtaataagtgagtctatttcgataaattcgattataaactgtagagttacttgcgattcccaatctcagtcctgcttttatgaaatattacttaaaatacctaatcatcatagcatggaatgttcttatcctggatcgtatgaaaatgaaatgttcatggaaaaatggagggatgagaattggtcaatcattataaaacattattgtttttaccttgtatttagctttgcatttgctggttgcgttgcatttgcgatctgcaaaaatctacgactgagtacaactatgaaattacttatgcttttgagtattttggtttgcttatctcagccaatcttgaataattga

SEQ ID NO: 281 - DNA, 510 bp, African swine fever virus
atgaaggtttttctaggacttttactaggtttttcaatcatcctcattcttacatatcaatcaccgacaacccagcatcctcctaaggaagagcttgcgtactggtgcacttatgcaaaatcttgtgacttctgctgggattgccaaaatgacacttgtataaataaggtaataaatgagtctatttcgataacttcgattgtaaactgtagagttactcgcgattcccagtcctgtttttatgatatatcagttaaaatacctaatcatcatagcatggaatgttcatatcctagactttatgaacatgaaatgtttatggaaaaatggagggatgagtattggccaatcattataaaacagtgttgtttttaccttgtatttagctttgcatttgctggttgcgttgcatttgcgatctgcaaaaacctacgactgcgtacaactataaaactacttattcttttgagtattttggtttggttatctcagccaatcttgaataattga

SEQ ID NO: 282 - DNA, 312 bp, African swine fever virus; identical to SEQ ID NO: 276

SEQ ID NO: 283 - DNA, 516 bp, African swine fever virus
atgaaggtttttctaggacttttactaggttattcaaccatcctcattcttacatatcaatcaccaacaacccagcatcctcctaaggaagagcttgagtattggtgcacttatgcaaaaacttgtgacttctgctgggattgccaaaatgacacttgtataaataaggtaataaatgaatctatttcgatgaattcgattgtaaactgtagagttactcgcgattcccaatcccagtcctgtttttatgaaatatcacttaaaatacctaattatcatagcatggaatgttcatatcctagactttataaacatttcatgtccatggaaaaatggagggatgagaattggccaatccttataagacattattgtttttaccttgtatttagctttgcatttgctggttgcgttgcatttgcgatctgcaaaaatctacgactgcgtacaaccatgaaattacttatgcttttgagtattttggttttgttatctcagccaatcttgaataattga

SEQ ID NO: 284 - DNA, 324 bp, African swine fever virus
atgaacgcaaccatgaaggtttttctaggacttttactaggttattcaaccatcctcattcttacatatcaatcaccgacaacccagtggtgtttttatgaaatatcacttaaaatacttaatcatcatagcatggaaaaatggagggataagaattggtcaatcattataaggtattattgtttttaccttgtgtttagctttgcatttgccggttgcgttgcatttgcgatctgcaaaaatctacgactgtgtacaaccatgaaattacttatgcttttgggtattttggttttgttatctcagccaatcttgaataattga

SEQ ID NO: 285 - DNA, 354 bp, African swine fever virus
atgaaggtttttctaggacttttactaggttattcaaccatcctcattcttacatatcaatcaccagcaacccagtggtgtttttatgaaatatcacttaaaatacctaatcatcatagcatggaatgttcatatcctagactttataaacatttcatattcatggaaaaatggagggataagaattggtcaatcattataaggtattattgtttttaccttgtatttagttttgcatttgctggttgcattgcatttgcgatctgcaaaaatctacgactgtgtacaactatgaaattacttatgcttttgagtattttggttttgttatctcagccaatcttgaataattga

SEQ ID NO: 286 - DNA, 354 bp, African swine fever virus
atgaaggtttttctaggacttttactaggttattcaaccatcctcattcttacatatcaatcaccagcaacccagtggtgtttttatgaaatatcacttaaaatacctaatcatcatagcatggaatgctcatatcctagactttataaacatttcatgttcatgaaaaaatggagggataagaattggtcaatcattataaggtattattgtttttaccttgtatttagttttgcatttgctggttgcattgcatttgcgatctgcaaaaatctacgactgtgtacaactatgaaattacttatgcttttgagtattttggttttgttatctcagccaatcttgaataattga

SEQ ID NO: 287 - DNA, 714 bp, African swine fever virus
atgttagaaatagtattggcaacgctgctaggcgacctgcagcggctccgggttcttacccctcagcagcgggcagttgccttctttcgagccaatactaaggagctagaggacttcttatgctcagatgggcagtctgaggaggtactgtctggcccccttcttaaccgtctactagaaccctcaggccctcttgatattttaaccggatatcacctatttcgtcagaatcccaaggcaggtcagttgcgcggccttgaggtcaagatgcttgaacggttatacgatgctaatatttacaatatactgtctcggctgcggcctgaaaaagttcgcaacaaggctattgagctatactgggttttccgagctatccatatttgtcatgctcctttagttttagatattgtacgatatgaggaaccggactttgctgaactggcctttatttgtgctgcttactttggtgaacctcaggtaatgtatttgctctacaaatatatgcctctgacccgcgcagttcttacggatgccatccggataagtcttgagagcaacaaccaggtagggatttgctatgcttacttgatgggaggcagcctcaagggactagtctccgccccactgcgtaaacgtctgcgcgccaaactacgctcgcagcgcaaaaagaaggacgttctttcaccccacgacttcttactgctgctccagtag

SEQ ID NO: 288 - DNA, 714 bp, African swine fever virus; identical to SEQ ID NO: 287

SEQ ID NO: 289 - DNA, 219 bp, African swine fever virus
atgcaaaataaaatcccaaattttaaccttttctttttttttctatacaggatgttagaaatagtattggcaacgctgctaggcgacctgcagcggctccgggttcttacccctcagcagcgggcggttgccttctttcgagccaatactaaggagctagaggacttcttacgctcagatgggcaatctgaggagatactgtctggcccccctccttaa

SEQ ID NO: 290 - DNA, 765 bp, African swine fever virus
atgcaaaataaaatcccaaattttaaccttttctttttttttctatacaggatgttagaaatagtattggcaacgctgctaggcgacctgcagcggctccgggttcttacccctcagcagcgggcggttgccttctttcgagccaatactaaggagctagaggacttcttacgctcagatgggcaatctgaggagatactgtctggccccctccttaaccgtctactagaaccctcatgccctcttgatattttaaccggatatcacctatttcgtcagaatcccaaggcaggtcagttgcgcggccttgaggtcaagatgcttgaacggttatacgatgctaatatttacaatatattgtctcggctgcgacctgaaaaagtccgcaacaaggctattgagctatactgggttttccgagctatccatatttgtcatgctcctttagttttagatattgtacgatatgaggaaccggactttgctgaactggcctttatttgtgctgcttactttggtgaacctcaggtaatgtatttgctctacaaatatatgcctctgacccgcgcagttcttacggatgccatccagataagtcttgagagcaacaaccaggtagggatttgctatgcttacttgatgggaggcagcctcaagggactagtctccgccccactgcgtaaacgtctgcgcgccaaactacgctcgcagcgcaaaaagaaggacgttctttcaccccacgacttcttactgctgctccagtag

SEQ ID NO: 291 - DNA, 765 bp, African swine fever virus
atgcaaaataaaatcccaaattttaaccttttctttttttttctatacaggatgttagaaatagtattggcaacgctgctaggcgacctgcagcggctccgggttcttacccctcagcagcgggcggttgccttctttcgagccaatactaaggagctagaggacttcttacgctcagatgggcaatctgaggagatactgtctggccccctccttaaccgtctactagaaccctcatgccctcttgatattttaaccggatatcacctatttcgtcagaatcccaaggcaggtcagttgcgcggccttgaggtcaagatgcttgaacggttatacgatgctaatatttacaatatattgtctcggctgcgacctaaaaaagtccgcaacaaggctattgagctatactgggttttccgagctatccatatttgtcatgctcctttagttttagatattgtacgatatgaggaaccggactttgctgaactggcctttatttgtgctgcttactttggtgaacctcaggtaatgtatttgctctacaaatatatgcctctgacccgcgcagttcttacggatgccatccagataagtcttgagagcaacaaccaggtagggatttgctatgcttacttgatgggaggcagcctcaagggactagtctccgccccactgcgtaaacgtctgcgcgccaaactacgctcgcagcgcaaaaagaaggacgttctttcaccccacgacttcttactgctgctccagtag

SEQ ID NO: 292 - DNA, 714 bp, African swine fever virus
atgttagaaatagtattggcaacgctgctaggtgacctgcagcggctccgggttcttacccctcagcagcgggcggttgccttctttcgagccaatactaaggagctagaggactttttatgctcagatgggcagtctgaggagatactgtccggccccctccttaaccgtctactagaaccctcaggccctcttgatattttaaccggatatcacttatttcgtcagaatcccaaggcaggtcaggtgcgcggccttgaggtcaagatgcttgaacggttatacgatgctaatatttacaatatattgtctcggctgcgacctgaaaaagttcgcaacaaggctgttgagctatactgggtttttcgggctatcaatatgtgtcatgctcctttagttttagatattgtacgatatgaggaaccggactttgctgaactggcctttatttgtgctgcttactttggtgaacctcaggtaatgtatttgctctacaaatatatgcctctgacccgcgcagttcttacggatgccatccagataagtcttgagagcaacagccaggtagggatttgctatgcctacttgatgggaggcagcctcaagggcctagtccgtgccccactacgtaaacgtctgcgcgccaaactacgctcgcagcgcaaaaagaaggacgttcttccaccccatgacttcttactgctgctccagtaa

SEQ ID NO: 293 - DNA, 714 bp, African swine fever virus
atgttagaaatagtattggcaacgctgctaggtgacctgcagcggctccgggttcttacccctcagcagcgggcggttgccttctttcgagccaatactaaggaggtagaagacttcttatgctcagatgggcagtctgaggaggtactgtccggccccctccttaaccgtctactagaaccctcaggccctcttgatattttaaccggatatcacttatttcgtcagaatcccaaggcgggtcaggtgcgcggccttgaggtcaagatgcttgaacggttatacgatgctaatatttacaatatattgtctcggctgcgacctgaaaaagttcgcaacaaggctgttgagctatactgggtttttcgggctatcaatatgtgtcatgctcctttagttttagatattgtacgatatgaggaaccggactttgctgaactggcctttatttgtgctgcttactttggtgaacctcaggtaatgtatttgctctacaaatatatgcctctgagccgcgcagttcttacggatgccatccagataagtcttgagagcaacagccaggtagggatttgctatgcctacttgatgggaggcagcctcaagggcctagtccgtgccccactacgtaaacgtctgcgcgccaaactacgctcgcagcgcaaaaagaaggacgttcttccaccccacgacttcttactgctgctccagtaa

SEQ ID NO: 294 - DNA, 714 bp, African swine fever virus
atgttagaaatagtattggcaacgctgctaggtgacctgcagcggctccgggttcttacccctcagcagcgggcggttgccttctttcgagccaatactaaagagctagaggacttcttatgtccagatgggcagtctgaggaggtgctgtctggctccctccttaaccgtctactagaaccctcaggccctcttgatattttaactggatatcacctatttcgtcagaatcccaaggcaggtcagttgcgcggccttgaggtcaagatgcttgaacggttatacgatgctaatatttacaacatattgtcccggctgcgacctgaaaaagttcgcaacaaggctgttgagctatactgggtttttcgggctatcaatatgtgtcatgctcctttagttttagatattgtacgaaatgaggaactggactttgctgaactggcctttatttgtgctgcttactttggtgagcctcaggtaatgtatttgctctacaaatatatgcctctgacccgcgcagttcttacggatgccatccagataagtcttgagagcaacagccaggtggggatttgctatgcctacttgatgggaggcagcctcaagggactagtccgtgccccgctacgtaaacgtctgcgcgccaaactacgctcgcagcgcaaaaagaaggatgttcttccaccccacgacttcttactgctgctccagtaa

SEQ ID NO: 295 - DNA, 774 bp, African swine fever virus
atgcaaaataaaatcccaaattttaaccttttctttttttttctatacaagatgttagaaatagtattggcaacgctgctgggtgatctgcagcaacttaaggatcttacacctaagcagcgggccgtagctttcttccgagccaacaccaaagagctagaggactttctctaccccgatgggcagactgaggagttactccctggatttcttcttaaccatttactagaaccctcaggccctattgaaattttaaccggatatcacctctttcgtcagaatccaaaggcaggtcgcttgcgaggccttgaggtgaaaatgcttgaacggttatatgatgctaatatttacaatatgctggctcggctacggcctgagttggttcgtgacaaggctgttgagctatattggctttttcgagctattttaatatgtcatggtcccttggttttggagattgtacgacatgagacgttggactttgcagaaaccgcctttatctgtgctgcttactttagtgaacctcaggtaatgtacgctctttataactttataccacccactcatgcagtccttgctgatgccatccagatgtgtcttgagagcaacagcgaggcagggatttgctatgtctacctaatgggaggtaacctcaagggcaaggtgcccggctcgctgcgcaaacgtctgcgtgccagtccacttcggcaagaacgcaaaaagaaaaacgtccttccgccccacgaattcctactcctgctccacgggatttaa

SEQ ID NO: 296 - DNA, 774 bp, African swine fever virus
atgcaaaataaaatcccaaattttaaccttttctttttttttctatacaagatgttagaaatagtattggccacgctgctaggtgatctgcagaaacttaaggatcttacacctccgcagcgggctgtggctttctttcgagccaacaccaaagagctagaggactttctctaccccgatgggcagtctgaggagttactccctggacttctccttaaccgtttactagaaccttcaggttctattgacattttaaccggttaccacctatttcgtgagaatccaaaggcaggtcgcttgcgaggccttgaggtgaaaatgcttgaacggttatatgatgctaatatttacaatatgctggctcggctacggcctgagttggttcgtgacaaggctattgagctatattggctctttcgggctattttaatgtgtcatagtcccttagttttggagattgtacgacatgagacaatggattttgcagaaaccgcctttatctgtgccgcttactttagtgaacctcaggtaatgtacgctctttataaatttatacctatctctcgtgcagtccttgctgatgccattcagatgtgtcttgagagcaacagcgaggcggggatttgctatgcttacctaatggggggtagcctcaagggcaaggtgcctggctcgctgcgcaaacgtctgcgtgccagtccacttcggcaagagcgcaaaaagaaaaacgtcctcccgccccatgaattcctactcatgctccatgggatttaa

SEQ ID NO: 297 - DNA, 765 bp, African swine fever virus
atgcaaaataaaatcccaaattttaaccttttctttttttttctatacaggatgttagaaatagtattggcaacgctgctaggtgacctgcagcggctccgggttcttacccctcagcagcgggcggttgccttctttcgagccaatactaaggagctagaggacttcttatgctcagatgggcagtctgaggagatactgtccggccccctccttaaccgtctactagaaccctcaggccctcttgatattttaaccggatatcacttatttcgtcagaatcccaaggcaggtcagttgcgcggtcttgaggtcaagatgcttgaacggttatacgatgctaatatttacaatatattgtctcggctgcgacctgaaaaagttcgcaacaaggctattgagctatactgggttttccgagctatccatatttgtcatgctcctttagttttagatattgtacgatatgaggaaccggactttgctgaactggcctttatttgtgctgcttactttggtgaacctcaggtaatgtatttgctctacaaatatatgcctctgacccgcgcagttcttacggatgccatccggataagtcttgagagtaacaaccaggtagggatttgctatgcttacttgatgggaggcagcctcaagggactagtctccgccccactgcgtaaacgtctgcgcgccaaactacgctcacagcgcaaaaagaaagacgttctttcaccccacgacttcttactgctgctccagtaa

SEQ ID NO: 298 - DNA, 765 bp, African swine fever virus
atgcaaaataaaatcccaaattttaaccttttctttttttttctatacaggatgttagaaatagtattggcaacgctgctaggcgacctgcagcggctccgggttcttacccctcagcagcgggcggttgccttctttcgagccaatactaaggagctagaggacttcttatgctcagatgggcagtctgaggagatactatctggcccccttcttaaccgtctactagaaccctcaggccctcttgatattttaactggatatcacctatttcgtcagaaccccaaggcaggtcagttacgcggccttgaggtcaagatgcttgaacggttatacgatgctaatatttacaatatattgtctcggctgcgacctgaaaaagttcgcaacaaggctattgagctttactgggtttttcgagctatccatatttgtcatgctcctttagttttagatattgtacgatatgaggaaccggactttgctgaactggcctttatttgtgctgcttactttggtgaacctcaggtaatgtatttgctctacaaatatatgcctctgacccgcgcagttcttacggatgccatccggataagtcttgagagtaacaaccaggtagggatttgctatgcttacttgatgggaggcagcctcaagggactagtctccgccccactgcgtaaacgtctgtgcgccaaactacgctcgcagcgcaaaaagaaggacgttctttcaccccacgacttcttactgctgctccagtaa

SEQ ID NO: 299 - DNA, 774 bp, African swine fever virus
atgcaaaataaaatcccaaattttaaccttttctttttttttctatacaagatgttagaaatagtattggcaacgctgctaggtgatctgcagaaacttaaagatcttacacctatgcagcgggctgtggctttctttcgagccaacactaaagagctagaggacttcctctgccccgatgggcagtctgaggagttactccctggacttctccttaaccgtttattagaaccttcaggtcctattgacattttaaccggttaccacctatttcgtgagaatccgaaggcaggtcggctgcgtggccttgaggtcaagctgcttgaacggttatatgatgctaacatctacaatatattggcccagctacggcctgagttggttcgcaataaggctgttgagttatattggctctttcgggctattttgatgtgtcatggtcccttagttttggagattgtacgacatgagacaatggattttgcagaattagcctttatctgtgctgcttactttagtgaacctcaggtaatgtacgctatttataaatttatacctatctcttgcgcagtccttgctgatgccattcagatgtgtcttgagagtaacagcgaggcggggatttgctatgcttacctaatggggggtagcctcaagggcaaggtgcctggcgcgctgcgcaaacgtctgcgtgccagtccacttcggcaagagcgcaaaaagaaaaacgttcttccaccccatgaattcctactcatgcttcatgggatttaa

SEQ ID NO: 300 - DNA, 723 bp, African swine fever virus
atgttagaaatagtattggcaacgctgctaggtgatctgcagaagcttagggatcttacacctatgcagcgggctgtggctttctttcgagtcaacactaaagagctagaggacttcctctaccccgatgggcagtctgaggagttgctccctggacttctccttaaccgtttactagaaccttcaggccctattgacattttaaccggttaccacctatttcgtgagaacccaaaggcaggtcggctgcgtggccttgaggttaagctgcttgaacggttatatgatgctaacatctacaatatgttggcccaaatacgacctgagttggttcgtatcaaggctattgagttatattggctctttcgggctattttaatgtgtcatagtcccttagttttggagattgtacgacatgagacaatggattttgcagaattagcctttatctgtgctgcttactttagtgaacctcaggtaatgtacgctctttataaatttatacctatttctcgcgcagtccttgctgatgccatcgagatgtctcttgagagcaacagcgagacggggatttgctatgcctacctaatggggggtagcctcaagggcaaggtgcccggcccgctgcgcaaacgtctgcgtgccagtccacttcggcaagagcgcaaaaagaaaaacgtccttccgccccatgaatttctactcatgctccatgggatttaa

SEQ ID NO: 301 - protein, 237 aa, African swine fever virus
MLEIVLATLLGDLQRLRVLTPQQRAVAFFRANTKELEDFLCSDGQSEEVLSGPLLNRLLEPSGPLDILTGYHLFRQNPKAGQLRGLEVKMLERLYDANIYNILSRLRPEKVRNKAIELYWVFRAIHICHAPLVLDIVRYEEPDFAELAFICAAYFGEPQVMYLLYKYMPLTRAVLTDAIRISLESNNQVGICYAYLMGGSLKGLVSAPLRKRLRAKLRSQRKKKDVLSPHDFLLLLQ

SEQ ID NO: 302 - protein, 237 aa, African swine fever virus; identical to SEQ ID NO: 301

SEQ ID NO: 303 - protein, 257 aa, African swine fever virus
MQNKIPNFNLFFFFLYKMLEIVLATLLGDLQKLKDLTPMQRAVAFFRANTKELEDFLCPDGQSEELLPGLLLNRLLEPSGPIDILTGYHLFRENPKAGRLRGLEVKLLERLYDANIYNILAQLRPELVRNKAVELYWLFRAILMCHGPLVLEIVRHETMDFAELAFICAAYFSEPQVMYAIYKFIPISCAVLADAIQMCLESNSEAGICYAYLMGGSLKGKVPGALRKRLRASPLRQERKKKNVLPPHEFLLMLHGI

SEQ ID NO: 304 - protein, 257 aa, African swine fever virus
MQNKIPNFNLFFFFLYKMLEIVLATLLGDLQQLKDLTPKQRAVAFFRANTKELEDFLYPDGQTEELLPGFLLNHLLEPSGPIEILTGYHLFRQNPKAGRLRGLEVKMLERLYDANIYNMLARLRPELVRDKAVELYWLFRAILICHGPLVLEIVRHETLDFAETAFICAAYFSEPQVMYALYNFIPPTHAVLADAIQMCLESNSEAGICYVYLMGGNLKGKVPGSLRKRLRASPLRQERKKKNVLPPHEFLLLLHGI

SEQ ID NO: 305 - protein, 254 aa, African swine fever virus
MQNKIPNFNLFFFFLYRMLEIVLATLLGDLQRLRVLTPQQRAVAFFRANTKELEDFLRSDGQSEEILSGPLLNRLLEPSCPLDILTGYHLFRQNPKAGQLRGLEVKMLERLYDANIYNILSRLRPEKVRNKAIELYWVFRAIHICHAPLVLDIVRYEEPDFAELAFICAAYFGEPQVMYLLYKYMPLTRAVLTDAIQISLESNNQVGICYAYLMGGSLKGLVSAPLRKRLRAKLRSQRKKKDVLSPHDFLLLLQ

SEQ ID NO: 306 - protein, 123 aa, African swine fever virus
MLVIFLGLLGLLANQVLGLPNQPAGQLHPTDNPPKEELGYWCTYTESCKFCWNCQNGLCEGKLENTTILENEYVQSCIVSRWLNKCMYDLGQGIDHVMACSEPKPWNPYKILKREWKKNVSQN

SEQ ID NO: 307 - protein, 123 aa, African swine fever virus
MLVIFLGLLGLLANQVLGLPNQPAGQLHPTDNPPKEELGYWCTYTESCKFCWNCQNGLCEGKLENTTILENEYVQSCIVSRWLNKCMYDLGQGIHHVMPCSEPKPWNPYKILKKEWKENVSQN

SEQ ID NO: 308 - protein, 120 aa, African swine fever virus
MLVIFLGILGLLANQVSSQLVGQLHPTENPSENELEYWCTYMECCQFCWDCQDGLCVNKLGNTTILENEYVHPCIVSRWLNKCMYDLGQGIDHVMVCSQPKYWNPYKILKKEWKENNSQN

SEQ ID NO: 309 - protein, 120 aa, African swine fever virus; identical to SEQ ID NO: 308

SEQ ID NO: 310 - protein, 121 aa, African swine fever virus
MLVIFLGILGLLANQVSSQLVGQLHPTENPSENELEYWCTYMECCQFCWDCQNGLCVNKLGNTTILENEYVHPCIVSRWLNKCMYDLGQGIDHVMVCSQPKYWNPYKILKKEWKENNSQNK

SEQ ID NO: 311 - protein, 118 aa, African swine fever virus
MLVIFLGILGLLVNQVSSQPVGQLHPTENPSENELEYWCTYMECCQFCWDCQNGLCVNKLGNTTILENEYVHPCIVSRWLNKCMYDLGQGIDHVMVCSQPKYWNPYKILKKEWKENNS

SEQ ID NO: 312 - protein, 121 aa, African swine fever virus; identical to SEQ ID NO: 310

SEQ ID NO: 313 - protein, 121 aa, African swine fever virus; identical to SEQ ID NO: 310

SEQ ID NO: 314 - protein, 205 aa, African swine fever virus
MLVIFLGILGLLASQVSSQLVGQLRPTEEPPEEELEYWCAYMESCQFCWDCQDGTCINKIDGSVIYKNEYVKSCLVSRWLDKCMYDLDKGIYHTMNCNQVLGLPNQPAGQLHPTDNPPQEELEYWCTYTENCKFCWNCQNGLCEGKLENTTILENEYVQSCIVSRWLNKCMYDLGQGIHHVMACSEPKPWNPYKILKREWKENNS

SEQ ID NO: 315 - protein, 205 aa, African swine fever virus; identical to SEQ ID NO: 314

SEQ ID NO: 316 - protein, 103 aa, African swine fever virus; identical to SEQ ID NO: 2

SEQ ID NO: 317 - protein, 119 aa, African swine fever virus
MKVFLGLLLGYSTILILTYQSPTTPWCFYEISLKIPNHHSMKCCSYPRLYEHEMFMEKWRDKNWPIIIRYYCFYLVFSFVFAGCVAFAICKNLRLSTTMKLLMLLSILVLLLSQPILNN

SEQ ID NO: 318 - protein, 119 aa, African swine fever virus; identical to SEQ ID NO: 317

SEQ ID NO: 319 - protein, 168 aa, African swine fever virus
MKVFLGLLLGFSIILILTYQSPTTQHPPKEELAYWCTYAKSCDFCWDCQNDTCINKVINESISITSIVNCRVTRDSQSCFYEISVKIPNHHSMECSYPRLYEHEMFMEKWRDEYWPIIIKQCCFYLVFSIAFAGCVAFAICKNLRLRTTIKLLILLSILVWLSQPVLN

SEQ ID NO: 320 - protein, 171 aa, African swine fever virus
MKVFLALLLGYLTILILTYQTPTTQHPPKEELPYWCTYVKNCDLCWDCQDSIYWNKVISESISINSIINCRVTCDSQSQSCFYEILLKIPNHHSMECSYPGSYENEMFMEKWRDENWSIIIKHYCFYLVFSFAFAGCVAFAICKNLRLSTTMKLLMLLSILVCLSQPILNN

SEQ ID NO: 321 - protein, 169 aa, African swine fever virus
MKVFLGLLLGFSIILILTYQSPTTQHPPKEELAYWCTYAKSCDFCWDCQNDTCINKVINESISITSIVNCRVTRDSQSCFYDISVKIPNHHSMECSYPRLYEHEMFMEKWRDEYWPIIIKQCCFYLVFSFAFAGCVAFAICKNLRLRTTIKLLILLSILVWLSQPILNN

SEQ ID NO: 322 - protein, 103 aa, African swine fever virus; identical to SEQ ID NO: 2

SEQ ID NO: 323 - protein, 171 aa, African swine fever virus
MKVFLGLLLGYSTILILTYQSPTTQHPPKEELEYWCTYAKTCDFCWDCQNDTCINKVINESISMNSIVNCRVTRDSQSQSCFYEISLKIPNYHSMECSYPRLYKHFMSMEKWRDENWPILIRHYCFYLVFSFAFAGCVAFAICKNLRLRTTMKLLMLLSILVLLSQPILNN

SEQ ID NO: 324 - protein, 107 aa, African swine fever virus
MNATMKVFLGLLLGYSTILILTYQSPTTQWCFYEISLKILNHHSMEKWRDKNWSIIIRYYCFYLVFSFAFAGCVAFAICKNLRLCTTMKLLMLLGILVLLSQPILNN

SEQ ID NO: 325 - protein, 117 aa, African swine fever virus
MKVFLGLLLGYSTILILTYQSPATQWCFYEISLKIPNHHSMECSYPRLYKHFIFMEKWRDKNWSIIIRYYCFYLVFSFAFAGCIAFAICKNLRLCTTMKLLMLLSILVLLSQPILNN

SEQ ID NO: 326 - protein, 117 aa, African swine fever virus
MKVFLGLLLGYSTILILTYQSPATQWCFYEISLKIPNHHSMECSYPRLYKHFMFMKKWRDKNWSIIIRYYCFYLVFSFAFAGCIAFAICKNLRLCTTMKLLMLLSILVLLSQPILNN

SEQ ID NO: 327 - protein, 210 aa, African swine fever virus
MIIILIFLICLKIVLNNNIIIWHTLNETIFLNNSITFTDNLGGLFWNTYYNNNHSILTSCGIYQNYCSCTNNYSTSLYNITNNCGLIIFPNNTKRFNTTYELVYLNNSTKYTINLLVPVTPPNITYNCTTSVITCKKNDGTNTNMFLKINDTIVKSSNESVFEYSWNSTDLNNNFTAICIINNTINSSNDTQTIECPNILLNNYLDFFQV

SEQ ID NO: 328 - protein, 207 aa, African swine fever virus
MIIILIFLIYLKLVLNNNIIIWHPTLNETIFLNVTTIGNISGLFWNTYYNNNRSELTKCGIYNNYCSCDSLNTSLYNINNCSLVIFPNNTKIFNTIYELIYSGNQTNYTIKLLESTTPPNITYNCTTSVITCKKNNGTNTNIFLTINNTIVNSTNEDVIYYYWNRSEFNNFTATCMINNTINSSNISEIIDCTNTLLLNSYLDFFQV

SEQ ID NO: 329 - protein, 211 aa, African swine fever virus
MIIKVIFLVFFKMVLSNIIIWSTLNNTIYLNINYSDSNLYTGLFWNKYYNNTRNNFTTCVTSNSSYCTCKGHNTSLYNITNNCSLIIFPNNTKIFNTTYELVYLNNKINYTIQMVQPVDPPIIIYNDTNNSLITCEKNNGTNTEIYLYLNDTFINNTNENSIKYYWNCSELNHNITTTCIINNTLNSANTTKVINCTNLLLKSDQNYFLKN

SEQ ID NO: 330 - protein, 205 aa, African swine fever virus
MIIKLIFLICFKIVLSINYWVRYNDTVTLNSNINSETEGIFWNFYNNTFNTIATCGKKNNVCECSNYDKSLYNITNNCSLTIFPNNTKIFNTTYQLVYSRNRINYTINLLLPVTSPIITYNCTQSLITCEKTNGTNIHLFLNLNDTINEYTNKSFLNYYWNSSELNNIFLATCIINNTLNSANTTKVINCTNPLLKSYQNYFLEN

SEQ ID NO: 331 - protein, 203 aa, African swine fever virus
MIILIFLIFSNIVLSIDYWVSFNKTIILDSNITNDNNDINGVSWNFFNNSFNTLATCGKAGNFCECSNYSTSIYNITNNCSLTIFPHNDVFDTTYQVVWNQIINYTIKLLTPATPPNITYNCTNFLITCKKNNGTNTNIYLNINDTFVKYTNESILEYNWNNSNINNFTATCIINNTISTSNETTLINCTYLTLSSNYFYTFF

SEQ ID NO: 332 - protein, 204 aa, African swine fever virus
MIIILIFLIIPNIVLSIDYWVSFNKTIILDSNITNDNNDINGVSWNFLNNSLNTLATCGKAGNFCECSNYSTSLYNIAHNCSLTIFPHNDVFGTPYQVVWNQIINYTIKLLTPVTPPNITYNCTNFLITCKKNNGTNTIIYFNINDTNVKYTNESILEYNWNNSNFNNFTATCIINNTINSSNDTQTIDCINTLLSSYLDFFQV

(19) United States
(12) Patent Application Publication
(10) Pub. No.: US 2023/0117978 A1
(43) Pub. Date: Apr. 20, 2023

(54) VACCINE AGAINST AFRICAN SWINE FEVER VIRUS INFECTION

(71) Applicants: THE PIRBRIGHT INSTITUTE, Woking (GB); Kumamoto University, Kumamoto (JP); THE CHANCELLOR MASTERS AND SCHOLARS OF THE UNIVERSITY OF OXFORD, Oxford (GB)

(72) Inventors: Linda Dixon, Woking (GB); Ana Reis, Woking (GB); Simon Davis, Oxford (GB); Yuan Jenq Lui, Oxford (GB); Shinji Ikemizu, Kumamoto (JP)

(21) Appl. No.: 17/905,488

(22) PCT Filed: Mar. 5, 2021
(86) PCT No.: PCT/GB2021/050560

§ 371 Date: Sep. 1, 2022

(30) Foreign Application Priority Data
Mar. 6, 2020 (GB) 2003289.2
Mar. 6, 2020 (GB) 2003292.6
Apr. 22, 2020 (GB) 2005878.0
Apr. 22, 2020 (GB) 2005880.6
Aug. 28, 2020 (GB) 2013541.4

(51) Int. Cl. A61K 39/12

(52) U.S. Cl. CPC A61K 39/12; A61K 2039/5254

(57) ABSTRACT

The present invention relates to attenuated African Swine Fever viruses. The attenuated viruses protect pigs against subsequent challenge with virulent virus. The present invention also relates to the use of such attenuated viruses to treat and/or prevent African Swine Fever.

### FIELD OF THE INVENTION

The present invention relates to attenuated African Swine Fever viruses. The attenuated viruses protect pigs against subsequent challenge with virulent virus. The present invention also relates to the use of such attenuated viruses to treat and/or prevent African Swine Fever.

### BACKGROUND TO THE INVENTION

### African Swine Fever (ASF)

African swine fever is a devastating haemorrhagic disease of domestic pigs caused by a double-stranded DNA virus, African swine fever virus (ASFV). ASFV is the only member of the Asfarviridae family and replicates predominantly in the cytoplasm of cells. Virulent strains of ASFV can kill domestic pigs within about 5-14 days of infection with a mortality rate approaching 100%.

ASFV can infect and replicate in warthogs (Phacochoerus sp.), bushpigs (Potamocherus sp.) and soft ticks of the Ornithodoros species (which are thought to be a vector), but in these species few if any clinical signs are observed and long term persistent infections can be established. ASFV was first described after European settlers brought pigs into areas endemic with ASFV and, as such, is an example of an “emerging infection”. The disease is currently endemic in many sub-Saharan countries and in Europe in Sardinia. Following its introduction to Georgia in the Trans Caucasus region in 2007, ASFV has spread extensively through neighbouring countries including the Russian Federation. In 2012 the first outbreak was reported in Ukraine and in 2013 the first outbreaks in Belarus. In 2014 further outbreaks were reported in pigs in Ukraine and detection in wild boar in Lithuania and Poland.

There is currently no treatment for ASF. Prevention in countries outside Africa has been attempted on a national basis by restrictions on incoming pigs and pork products, compulsory boiling of waste animal products under licence before feeding to pigs and the application of a slaughter policy when the disease is diagnosed. Prevention in Africa is based on measures to keep warthogs and materials contaminated by warthogs away from the herd.

There is thus a need for improved measures to control ASFV infection and prevent spread of the disease.

### African Swine Fever Virus (ASFV)

The complete genome sequences of ASFV isolate Benin 97/1 (a highly pathogenic virus from West Africa, Group1), isolate OURT88/3 (non-pathogenic, attenuated virus from Portugal, Group 1) and isolate BA71V (Vero cell tissue culture adapted non-pathogenic virus, Group 1) have been compared (Chapman et al. 2008 J. Gen. Virol. 89: 397-408).

In the OURT88/3 genome, the multigene family (MGF) 360 18R (DP148R) gene, EP153R gene and EP402R gene are each interrupted by frameshift mutations. Additionally, the following MGF genes are absent from the OURT88/3 genome: MGF 110 3L, 6L, 7L, 8L, 10L, 11L and 12L, MGF 300 3L, MGF 360 5L, 6L, 7L, 10L, 11L, 12L, 13L, 14L, 20R, 21R and 22R, and MGF 505 1R, 2R and 6R. The MGF 505 3R gene is also truncated.

The sequences of the high-virulence Lisboa60 strain and the low-virulence NH/P68 strain have also been compared (Portugal et al. 2015 J. Gen. Virol. 96: 408-419).

In the NH/P68 genome, the MGF 360 18R (DP148R) gene, EP153R gene and EP402R gene are each interrupted by a premature stop codon. Additionally, the following MGF genes are absent from the NH/P68 genome: MGF 110 3L, 6L, 7L, 8L, 10L, 11L and 12L, MGF 360 5L, 6L, 7L, 10L, 11L, 12L, 13L, 14L, 20R, 21R and 22R, and MGF 505 1R, 2R and 6R. The MGF 360 9L and MGF 505 3R genes are also truncated.

Deletion of the DP148R gene from the virulent Benin 97/1 isolate reduced virulence and induced protection against challenge with the parental virus (Reis et al. 2017 J. Virol. 91, 24: e01428-17).

### SUMMARY OF THE INVENTION

Generally, the invention relates to an attenuated African Swine Fever virus in which expression and/or activity of the genes DP148R, EP153R and EP402R is disrupted, whilst expression and/or activity of particular MGF genes is not disrupted.

The invention provides an attenuated African Swine Fever (ASF) virus in which the expression and/or activity of the genes DP148R, EP153R and EP402R is disrupted; and which comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

In a further aspect the invention provides an attenuated African Swine Fever (ASF) virus in which the expression and/or activity of the genes DP148R, EP153R and EP402R is disrupted;

and which comprises a functional version of one or more of the following genes:
multigene family (MGF) 110 11L and 12L,
MGF 360 6L, 10L, 11 L, 12L, 13L, and 14L, and
MGF 505 1R and 2R.

In a further aspect the invention provides an attenuated African Swine Fever (ASF) virus in which the expression and/or activity of the genes DP148R, EP153R and EP402R is disrupted;

and which comprises a functional version of one or more of the following genes:
multigene family (MGF) 110 5L, 6L, 8L and 12L,
MGF 360 6L, 10L, 11 L, 12L, 13L, 14L and 21R, and
MGF 505 1R and 2R.

The invention also provides a vaccine comprising an attenuated ASF virus of the invention.

The invention further provides a vaccine of the invention for use in treating and/or preventing ASF in a subject.

The invention further provides a method for treating and/or preventing ASF in a subject which comprises the step of administering to the subject an effective amount of a vaccine according to the invention.

The invention yet further provides a method of attenuating an ASF virus which comprises the step of disrupting the expression and/or activity of the following genes: DP148R, EP153R and EP402R.

### DESCRIPTION OF THE FIGURES

FIG. 1 shows confocal microscopy images of non-permeabilised cells expressing wild-type (A) or mutant (B) CD2v stained with sera from pigs immunised with attenuated ASFV containing a wild-type CD2v gene to detect surface expression.

FIG. 2 shows exemplary images from a HAD assay. Vero cells were infected with modified vaccinia virus Ankara expressing T7RNA polymerase and transfected with plasmids (pcDNA3) expressing wild-type or mutant CD2v full-length proteins with a C-terminal HA epitope tag. Pig red blood cells were added and cells observed for attachment of red blood cells to the surface. HAD of red blood cells is observed around three cells transfected with a plasmid expressing wild-type Benin CD2v (A). Partial HAD is observed around one cell expressing CD2v with the Y102 residue mutated to D (B). No HAD is observed for cells expressing CD2v with residue E99 mutated to R (C).

FIG. 3 depicts an alignment of the amino acid sequence of CD2v ligand-binding domain from different ASFV isolates of varying genotypes. E99 and equivalent residues in other isolates are highlighted in yellow.

FIG. 4 shows exemplary images from a HAD assay. Vero cells were infected with modified vaccinia virus Ankara expressing T7RNA polymerase and transfected with plasmids (pcDNA3) expressing wild-type or mutant CD2v full-length proteins from Benin or Georgia strains with a C-terminal HA epitope tag. Pig red blood cells were added and cells observed for attachment of red blood cells to the surface. HAD of red blood cells is observed around four cells transfected with a plasmid expressing wild-type Benin CD2v (A) and around four cells transfected with a plasmid expressing wild-type Georgia CD2v (B). No HAD is observed for cells expressing Georgia CD2v with residue Q96 mutated to R (C) or for untransfected Vero cells (D).

FIG. 5 shows WSL cells expressing CD2vE99R infected with BeninΔCD2v virus (A) or OURT88/3 virus (B), which has frame-shift interruptions in the EP153R and EP402R/CD2v genes. HAD is observed upon infected with BeninΔCD2v but not with OURT88/3.

FIG. 6 shows porcine marrow cells infected with ASFV Benin 97/1 wild-type (B left panel) or ASFV Benin 97/1 ΔDP148RΔEP153R-CD2vE99R (A, B right panel).

FIG. 7 shows levels of luciferase expression from an NF-κB dependent promoter in HEK293T cells transfected with an empty pcDNA3 vector (negative control) or a plasmid expressing DP148R. Cells were stimulated with IL-1β (A) or TNF-α (B) to induce NF-κB activation. Alternatively, NF-κB activation was induced by overexpression of the the two main kinases of the NF-κB pathway, IKKα (C) or IKKβ (D).

FIG. 8 shows immunofluorescence images of HeLa cells transfected with HA-tagged DP148R and stained for HA tag (red) and NF-κB p65 sub-unit (green). Nuclei are shown in blue. Cells were unstimulated (top row) or stimulated with TNFα (middle row) or IL-1β (bottom row) to induce NF-κB activation and p65 translocation to the nucleus. Arrows point to cells expressing DP148R. In stimulated cells expressing DP148R, p65 does not translocate to the nucleus.

FIG. 9 depicts the experimental protocol used to immunise, boost and challenge pigs with BeninΔDP148RΔEP153RΔCD2v (Group A) and BeninΔDP148RΔEP153R-CD2vE99R (Group B) viruses.

FIG. 10 shows rectal temperatures of pigs in Group A (A) and B (B) and the control Group E (C) during immunisation and challenge.

FIG. 11 shows clinical scores of pigs in Group A (A) and B (B) and the control Group E (C) during immunisation and challenge.

FIG. 12 shows viremia of pigs in Group A (A) and B (B) during immunisation and challenge.

FIG. 13 shows the antibody response of pigs in Group A (A) and B (B) during immunisation and challenge measured using a commercially available competitive ELISA based on the VP72 (B646L) major capsid protein.

FIG. 14 shows the cell-mediated immune response in Group A (A) and B (B) during immunisation and challenge. Peripheral blood mononuclear cells were collected pre-immunization, boost and challenge from pigs in Groups A and B and stimulated with ASFV Benin virus. Numbers of IFN gamma producing cells were measured by Elispot assay.

FIG. 15 shows the number of ASFV specific lesions found in pigs from Groups E, A and B at post-mortem.

FIG. 16 directly compares virus genome in blood after immunisation with BeninΔDP148R (A), BeninΔDP148RΔCD2v (B), BeninΔDP148RΔEP153RΔCD2v (C) and BeninΔDP148RΔEP153R-CD2vE99R (D).

### DETAILED DESCRIPTION

### African Swine Fever Virus (ASFV)

African swine fever virus (ASFV) is the causative agent of African swine fever (ASF). The genome structure of ASFV is known in the art, as detailed in Chapman et al. 2008 J. Gen. Virol. 89: 397-408. ASFV is a large, icosahedral, double-stranded DNA virus with a linear genome containing at least 150 genes. The number of genes differs slightly between different isolates of the virus. ASFV has similarities to the other large DNA viruses, e.g., poxvirus, iridovirus and mimivirus. In common with other viral haemorrhagic fevers, the main target cells for replication are those of monocyte, macrophage lineage.

Based on sequence variation in the C-terminal region of the B646L gene encoding the major capsid protein p72, 22 ASFV genotypes (I-XXII) have been identified. All ASFV p72 genotypes have been circulating in eastern and southern Africa. Genotype I has been circulating in Europe, South America, the Caribbean and western Africa. Genotype VIII is confined to four East African countries.

Examples of strains from some of the genotypes are given below:

Genotype I : OURT88/3; Brazil/79; Lisbon/60; BA715; Pret; Benin 97/1; IC/1/96; IC/576; CAM/82; Madrid/62; Malta/78; ZAR85; Katange63; Togo; Dakar59; Ourt88/1; BEN/1/97; Dom_Rep; VAL/76; IC/2/96; Awoshie/99; NIG/1/99; NIG/1/98; ANG/70; BEL/85; SPEC120; Lisbon/57; ASFV-Warm; GHA/1/00; GAM/1/00; Ghana; HOL/86; NAM/1/80; NUR/90/1; CAM/4/85; ASFV-Teng; Tegani; ASFV-E75.

Genotype II: Georgia 2007/1; POL/2015/Podlaskie (Polish strain); Belgium/Etalle/wb/2018; ASFV/Kyiv/2016/131; China/2018/AnhuiXCGQ

Genotype III: BOT 1/99

Genotype IV: ASFV-War; RSA/1/99/W

Genotype VI: MOZ 94/1

Genotype VII: VICT/90/1; ASFV-Mku; RSA/1/98

Genotype VIII: NDA/1/90; KAL88/1; ZAM/2/84; JON89/13; KAV89/1; DEZda; AFSV-Mal; Malawi LIL 20/1

Genotype IX: UGA/1/95

Genotype X: BUR/1/84; BUR/2/84; BUR/90/1; UGA/3/95; TAN/Kwh12; Hindell; ASFV-Ken; Virulent Uganda 65.

### ASFV Isolates

The attenuated ASF virus of the present invention may be derivable from a wild-type ASF virus isolate, but includes mutations in its genome such that the expression and/or activity of the genes DP148R, EP153R and EP402R is disrupted.

The term “wild-type” indicates that the virus existed (at some point) in the field, and was isolated from a natural host, such as a domestic pig, tick or warthog. ASFV isolates described to date are summarised in Table 1 below, together with their Genbank Accession numbers.

TABLE 1

| | | | | | GenBank |
|---|---|---|---|---|---|
| | | | | | accession |
| Isolate | Country | Host | Year | Virulence | no. |
| BA71qqV | Spain | Pig | 1971 | Tissue | U18466 |
| | | | | culture | |
| | | | | adapted | |
| Benin 97/1 | Spain | Pig | 1997 | High | AM712239 |
| Georgia 2007/1 | Georgia | Pig | 2007 | High | FR682468 |
| Kenya | Kenya | Pig | 1950 | High | AY261360 |
| Malawi Lil20/1 | Malawi | Tick | 1983 | High | AY261361 |
| Mkuzi | Zululand | Tick | 1978 | Unknown | AY261362 |
| OURT88/3 | Portugal | Tick | 1988 | Low | AM712240 |
| Pretorisuskop/96/4 | South Africa | Tick | 1996 | High | AY261363 |
| Tangani 62 | Malawi | Pig | 1962 | High | AY261364 |
| Warmbaths | South Africa | Tick | 1987 | Unknown | AY261365 |
| Warthog | Namibia | Warthog | 1980 | Unknown | AY261366 |
| Pol16_20186_07 | Poland | Pig | 2018 | High | MG939583 |
| Pig/HLJ/2018 | China | Pig | 2018 | Virulent | MK333180 |
| DB/LN/2018 | China | Dried | 2018 | Unknown | MK333181 |
| | | blood pig | | | |
| | | feed | | | |
| Belgium 2018/1 | Belgium | Wild boar | 2018 | Virulent | LR536725 |
| China/ | China | Pig | 2018 | Virulent | MK128995 |
| 2018/AnhuiXCGQ | | | | | |
| Ken05/TK1, | Kenya | | 2005/06 | Unknown | NC_044945 |
| Ken06 Bus | | | | | KM111295 |
| R35, R25, R7, R8, N10 | Uganda | | 2018 | Unknown | MH025920, |
| | | | | | MH025918, |
| | | | | | MH025917, |
| | | | | | MH025916, |
| | | | | | MH025919 |
| Pol_17_03029_C201 | Poland | Pig | 2017 | Unknown | MG939587 |
| 26544/OG10 | Sardinia/Italy | Pig | 2017 | Unknown | KM102979 |
| 47/Ss?2008 | Sardinia/Italy | Pig | 2008 | Unknown | KX354450 |
| Belgium/Etalle/wb/2018 | Belgium | Wild boar | 2018 | Virulent | MK543947 |
| ASFV/Kyiv/2016/131 | Ukraine | Pig | 2016 | Unknown | MN194591 |
| ASFV-SY18 | China | Pig | 2018 | Unknown | MH766894 |
| ASFV_HU_2018 | Hungary | Wild boar | 2019 | Unknown | MN715134 |
| ASFV-wbBS01 | China | Wild boar | 2019 | Unknown | MK645909 |
| ASFV Georgia 2007/1 | Georgia | Pig | 2007 | Virulent | LR743116 |
| (new version) | | | | | |
| ASFV/pig/China/ | China | Pig | 2019 | Unknown | MN172368 |
| CAS19-01/2019 | | | | | |
| ASFV/LT14/1490 | Latvia | Wild boar | 2019 | Unknown | MK628478 |
| Odintsovo_02/14 | Russia | Pig | 2014 | | NC_044948 |
| ASFV CzechRepublic | Czech | Wild boar | 2017 | Unknown | LR722600 |
| 2017/1 | Republic | | | | |
| ASFV Moldova 2017/1 | Moldova | Pig | 2019 | Unknown | LR722599 |
| RSA_2_2008 | South Africa | Pig | 2008 | Unknown | MN336500 |
| LIV_5_40 | Zambia | Tick | | Unknown | MN318203 |
| ASFV/POL/2015/ | Poland | Wild boar | 2015 | Virulent | MH681419 |
| Podlaskie | | | | | |

The genome of the attenuated ASFV of the invention may correspond to any ASFV genotype. The genome of the attenuated ASFV of the invention may essentially correspond to any ASFV genotype.

The term “corresponds to” means that the remainder of the genome of the attenuated ASFV of the invention is the same as a wild-type strain (i.e. a virus that existed at some point in the field). “The remainder of the genome” refers to all genes other than the disrupted genes DP148R, EP153R and EP402R. In other words, the genes of the attenuated ASFV of the invention may be the same as the genes of the wild-type strain, except for the genes DP148R, EP153R and EP402R. In an embodiment the genes of the attenuated ASFV of the invention are the same as the genes of the wild-type strain, except for DP148R, EP153R and EP402R.

The disrupted genes DP148R, EP153R and EP402R may also correspond to the wild-type strain. In an embodiment the genes DP148R, EP153R and EP402R correspond to the wild-type strain. In such an embodiment (i.e. where DP148R, EP153R and EP402R correspond to the wild-type strain) expression and/or activity of DP148R, EP153R and EP402R may be disrupted by one or more mutation in an intergenic region such as a promoter. In other words, the DP148R, EP153R and EP402R genes are the same as in the wild-type genome but their expression or activity is altered by mutation of a non-genic sequence. Thus all of the genes of the attenuated ASFV of the invention may be the same as the genes of the wild-type strain. In an embodiment all genes of the attenuated ASFV of the invention are the same as the genes of the wild-type strain.

The term “essentially corresponds to” means the same as “corresponds to” with the additional exception that the remainder of the genome may comprise one or more mutations. The one or more mutations may be in other genes (i.e. not in the genes DP148R, EP153R and EP402R).

The genome of the attenuated ASFV may correspond or essentially correspond to genotype I. The genome of the attenuated ASFV may correspond or essentially correspond to genotype II. The genome of the attenuated ASFV may correspond or essentially correspond to genotype III. The genome of the attenuated ASFV may correspond or essentially correspond to genotype IV. The genome of the attenuated ASFV may correspond or essentially correspond to genotype V. The genome of the attenuated ASFV may correspond or essentially correspond to genotype VI. The genome of the attenuated ASFV may correspond or essentially correspond to genotype VII. The genome of the attenuated ASFV may correspond or essentially correspond to genotype VIII. The genome of the attenuated ASFV may correspond or essentially correspond to genotype IX. The genome of the attenuated ASFV may correspond or essentially correspond to genotype X.

The genome of the attenuated ASFV may correspond or essentially correspond to genotype I. The genome of the attenuated ASFV may correspond or essentially correspond to genotype II.

Preferably, the genome of the attenuated ASFV may correspond or essentially correspond to genotype I.

The genome of the attenuated ASFV of the invention may correspond or essentially correspond to that of a virulent ASFV strain. Known virulent ASF virus strains include: Georgia 2007/1, Benin 97/1, Kenyan, Malawi Lil20/1, Pretorisuskop/96/4 and Tengani 62. The genome of the attenuated ASFV may correspond or essentially correspond to that of the Benin 97/1 strain. The genome of the attenuated ASFV may correspond or essentially correspond to that of the Georgia 2007/1 strain.

The genome of the attenuated ASFV may correspond or essentially correspond to that of the Benin 97/1 strain.

The genome of the attenuated ASFV of the invention may correspond or essentially correspond to that of an ASFV strain whose virulence is currently unknown, for example: Mkuzi, Warmbaths and Warthog.

In an embodiment the genome of the attenuated ASFV of the invention does not correspond to that of OURT88/3. In an embodiment the genome of the attenuated ASFV of the invention does not correspond to that of NH/P68. In an embodiment the attenuated ASFV of the invention is not OURT88/3. In an embodiment the attenuated ASFV of the invention is not NH/P68. In an embodiment the attenuated ASFV of the invention is neither OURT88/3 nor NH/P68.

The complete genome for the African swine fever virus Benin 97/1 pathogenic isolate is given in Genbank Locus: AM712239.1. The complete BA71 isolate genome encodes 151 open reading frames (ORFs), the Benin 97/1 isolate encodes 157 ORFs and the OURT88/3 isolate encodes 151 ORFs.

### Multigene Families (MGFs)

ASFV contains five multi-gene families which are present in the left and right variable regions of the genome. The MGFs are named after the average number of codons present in each gene: MGF100, 110, 300, 360 and 505/530. The N-terminal regions of members of MGFs 300, 360 and 505/530 share significant similarity with each other. It has been shown the MGF 360 and 505 families encode genes essential for host range function that involves promotion of infected-cell survival and suppression of type I interferon response.

The attenuated ASFV according to the present invention comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

In a further aspect the invention provides an attenuated African Swine Fever (ASF) virus in which the expression and/or activity of the genes DP148R, EP153R and EP402R is disrupted;

and which comprises a functional version of one or more of the following genes:
multigene family (MGF) 110 11L and 12L,
MGF 360 6L, 10L, 11 L, 12L, 13L, and 14L, and
MGF 505 1R and 2R.

Suitably, in one embodiment the invention provides an ASFV in which the expression and/or activity of the genes DP148R, EP153R and EP402R is disrupted;

and which comprises a functional version of one or more of the following genes:
multigene family (MGF) 110 5L, 6L, 8L, and 12L, and
MGF 360 6L, 10L, 11 L, 12L, 13L, 14L and 21R, and
MGF 505 1R and 2R.

Suitably, in one embodiment the invention provides an ASFV in which the expression and/or activity of the genes DP148R, EP153R and EP402R is disrupted;

and which comprises a functional version of one or more of the following genes:
multigene family (MGF) 110 6L, 8L, and 12L, and
MGF 360 6L, 10L, 11 L, 12L, 13L, 14L and 21R, and
MGF 505 1R and 2R.

Suitably, in one embodiment the invention provides an ASFV in which the expression and/or activity of the genes DP148R, EP153R and EP402R is disrupted;

and which comprises a functional version of one or more of the following genes:
multigene family (MGF) 110 12L, and
MGF 360 6L, 10L, 11 L, 12L, 13L, 14L, and
MGF 505 1R and 2R.

Suitably, in one embodiment the invention provides an ASFV in which the expression and/or activity of the genes DP148R, EP153R and EP402R is disrupted;

and which comprises a functional version of one or more of the following genes:
multigene family (MGF) 360 6L, 10L, 11 L, 12L, 13L, and 14L, and
MGF 505 1R and 2R.

The location of some of these genes in the genomes of a variety of ASFV strains is provided below in Table 2. The sequence identity of each gene to the corresponding Benin 97/1 gene is also provided.

TABLE 2a
MGF 360-10L

| | Start Nucleotide | Stop Nucleotide | % Nucleotide |
|---|---|---|---|
| Strain | Number | number | Identity |
| Benin 97/1 | 20677 | 19607 | 100 |
| Georgia 2007/1 | 26438 | 25368 | 93 |
| BA71V | Deleted | | |
| OURT88/3 | Deleted | | |
| Kenya 1950 | 30628 | 29563 | 82 |
| Malawi-Lil/20 | 24802 | 23763 | 91 |
| Mkuzi 1979 | 28139 | 27069 | 99 |
| Pretorisuskop/96/4 | 27186 | 26116 | 96 |
| Tengani 62 | 21600 | 20530 | 92 |
| Warmbaths | 26361 | 25322 | 94 |
| Warthog | 23130 | 22091 | 95 |
| E75 | 19981 | 18911 | 100 |

TABLE 2b
MGF 360-11L

| | Start Nucleotide | Stop Nucleotide | % Nucleotide |
|---|---|---|---|
| Strain | Number | number | Identity |
| Benin 97/1 | 21764 | 20703 | 100 |
| Georgia 2007/1 | 27526 | 26465 | 97 |
| BA71V | Deleted | | |
| OURT88/3 | Deleted | | |
| Kenya 1950 | 31716 | 30655 | 84 |
| Malawi-Lil/20 | 25892 | 24831 | 86 |
| Mkuzi 1979 | 29225 | 28164 | 99 |
| Pretorisuskop/96/4 | 28275 | 27214 | 97 |
| Tengani 62 | 22688 | 21627 | 97 |
| Warmbaths | 27449 | 26388 | 97 |
| Warthog | 24218 | 23157 | 97 |
| E75 | 21068 | 20007 | 100 |

TABLE 2c
MGF 360-12L

| | Start Nucleotide | Stop Nucleotide | % Nucleotide |
|---|---|---|---|
| Strain | Number | number | Identity |
| Benin 97/1 | 24668 | 23616 | 100 |
| Georgia 2007/1 | 30434 | 29382 | 96 |
| BA71V | Deleted | | |
| OURT88/3 | Deleted | | |
| Kenya 1950 | 34566 | 33549 | 90 |
| Malawi-Lil/20 | 28731 | 27682 | 92 |
| Mkuzi 1979 | 32125 | 31073 | 97 |
| Pretorisuskop/96/4 | 31150 | 30098 | 95 |
| Tengani 62 | 25592 | 24540 | 96 |
| Warmbaths | 30346 | 29294 | 96 |
| Warthog | 27088 | 26036 | 96 |
| E75 | 23971 | 22920 | 99 |

TABLE 2d
MGF 360-13L

| | Start Nucleotide | Stop Nucleotide | % Nucleotide |
|---|---|---|---|
| Strain | Number | number | Identity |
| Benin 97/1 | 25901 | 24840 | 100 |
| Georgia 2007/1 | 31656 | 30595 | 97 |
| BA71V | Deleted | | |
| OURT88/3 | Deleted | | |
| Kenya 1950 | 35812 | 34757 | 90 |
| Malawi-Lil/20 | 29980 | 28925 | 91 |
| Mkuzi 1979 | 33347 | 32286 | 99 |
| Pretorisuskop/96/4 | 32368 | 31307 | 95 |
| Tengani 62 | 26814 | 25753 | 95 |
| Warmbaths | 31559 | 30498 | 95 |
| Warthog | 28338 | 27277 | 95 |
| E75 | 25204 | 24143 | 99 |

TABLE 2e
MGF 360-14L

| | Start Nucleotide | Stop Nucleotide | % Nucleotide |
|---|---|---|---|
| Strain | Number | number | Identity |
| Benin 97/1 | 27146 | 26073 | 100 |
| Georgia 2007/1 | 32913 | 31840 | 99 |
| BA71V | Deleted | | |
| OURT88/3 | Deleted | | |
| Kenya 1950 | 37194 | 36121 | 92 |
| Malawi-Lil/20 | 31266 | 30193 | 92 |
| Mkuzi 1979 | 34620 | 33547 | 99 |
| Pretorisuskop/96/4 | 33598 | 32525 | 97 |
| Tengani 62 | 28056 | 26983 | 97 |
| Warmbaths | 32820 | 31747 | 96 |
| Warthog | 29568 | 28495 | 97 |
| E75 | 26449 | 25376 | 100 |

TABLE 2f
MGF 505-1R

| | Start Nucleotide | Stop Nucleotide | % Nucleotide |
|---|---|---|---|
| Strain | Number | number | Identity |
| Benin 97/1 | 21971 | 23566 | 100 |
| Georgia 2007/1 | 27734 | 29329 | 95 |
| BA71V | Deleted | | |
| OURT88/3 | Deleted | | |
| Kenya 1950 | 31904 | 33496 | 91 |
| Malawi-Lil/20 | 26041 | 27627 | 93 |
| Mkuzi 1979 | 29425 | 31020 | 96 |
| Pretorisuskop/96/4 | 28449 | 30044 | 94 |
| Tengani 62 | 22891 | 24486 | 95 |
| Warmbaths | 27651 | 29246 | 95 |
| Warthog | 24387 | 25982 | 95 |
| E75 | 21275 | 22870 | 100 |

TABLE 2g
MGF 505-2R

| | Start Nucleotide | Stop Nucleotide | % Nucleotide |
|---|---|---|---|
| Strain | Number | number | Identity |
| Benin 97/1 | 27352 | 28932 | 100 |
| Georgia 2007/1 | 33119 | 34699 | 99 |
| BA71V | 17725 | 19304 | 99 |
| OURT88/3 | 29532 | 29981 | 76 |
| Kenya 1950 | 37419 | 38985 | 90 |
| Malawi-Lil/20 | 31541 | 33121 | 93 |
| Mkuzi 1979 | 34826 | 36406 | 99 |
| Pretorisuskop/96/4 | 33795 | 35374 | 97 |
| Tengani 62 | 28261 | 29830 | 98 |
| Warmbaths | 33029 | 34597 | 96 |
| Warthog | 29773 | 31352 | 97 |
| E75 | 26655 | 28236 | 99 |

TABLE 2h
MGF 505-3R

| | Start Nucleotide | Stop Nucleotide | % Nucleotide |
|---|---|---|---|
| Strain | Number | number | Identity |
| Benin 97/1 | 29019 | 29861 | 100 |
| Georgia 2007/1 | 34786 | 35625 | 96 |
| BA71V | 20398 | 21915 | 100 |
| OURT88/3 | 20850 | 22367 | 100 |
| Kenya 1950 | 40295 | 41815 | 89 |
| Malawi-Lil/20 | 34322 | 35842 | 89 |
| Mkuzi 1979 | 37500 | 39017 | 99 |
| Pretorisuskop/96/4 | 36480 | 37985 | 89 |
| Tengani 62 | 30926 | 32443 | 90 |
| Warmbaths | 35712 | 37232 | 93 |
| Warthog | 32449 | 33954 | 90 |
| E75 | 29330 | 30847 | 100 |

The gene (i.e. nucleotide) sequences of these genes from different strains are given below.

MGF110 5L gene sequences
SEQ ID No. 266
China/2018/AnhuiXCGQ (genotype II) 9145-8528 MGF 110 5L/6L fusion
ATGTTGGTGATCTTCTTGGGAATTCTTGGCCTTCTGGCCAGCCAGGTTTCAAGTCAGCTCGTTGGACAACTTCGTCCAACAGA
GGAGCCTCCAGAGGAAGAACTCGAATACTGGTGTGCCTACATGGAAAGTTGCCAATTTTGCTGGGACTGCCAAGACGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAGGGTATCTACCATACCATGAATTGCAACCAGGTCTTAGGGCTACCTAATCAACCAGCTGGACAACT
TCATCCAACGGATAATCCTCCACAAGAAGAACTTGAATACTGGTGCACTTATACAGAAAACTGCAAGTTTTGCTGGAATTGTC
AAAATGGCCTTTGTGAGGGCAAATTGGAGAATACAACAATTCTTGAAAATGAGTATGTGCAATCATGTATAGTTTCCCGCTGG
CTAAATAAATGTATGTATGATCTAGGACAGGGGATTCACCATGTAATGGCCTGTTCTGAACCAAAGCCCTGGAATCCTTACAA
AATCTTAAAGAGAGAGTGGAAAGAAAATAATAGTTAA
SEQ ID No. 267
Georgia 2007/1 (genotype II) 9140-8523 MGF 110 5L/6L fusion
ATGTTGGTGATCTTCTTGGGAATTCTTGGCCTTCTGGCCAGCCAGGTTTCAAGTCAGCTCGTTGGACAACTTCGTCCAACAGA
GGAGCCTCCAGAGGAAGAACTCGAATACTGGTGTGCCTACATGGAAAGTTGCCAATTTTGCTGGGACTGCCAAGACGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAGGGTATCTACCATACCATGAATTGCAACCAGGTCTTAGGGCTACCTAATCAACCAGCTGGACAACT
TCATCCAACGGATAATCCTCCACAAGAAGAACTTGAATACTGGTGCACTTATACAGAAAACTGCAAGTTTTGCTGGAATTGTC
AAAATGGCCTTTGTGAGGGCAAATTGGAGAATACAACAATTCTTGAAAATGAGTATGTGCAATCATGTATAGTTTCCCGCTGG
CTAAATAAATGTATGTATGATCTAGGACAGGGGATTCACCATGTAATGGCCTGTTCTGAACCAAAGCCCTGGAATCCTTACAA
AATCTTAAAGAGAGAGTGGAAAGAAAATAATAGTTAA
SEQ ID No. 268
Ken05/Tk1 9698-9327 MGF 110 5L
ATGTTGGTGATCTTCTTGGGACTCCTTGGCCTGCTGGCCAACCAGGTCTTAGGGCTACCTAATCAACCAGCTGGACAACTTCA
TCCAACGGATAATCCTCCAAAAGAAGAACTTGGATACTGGTGCACTTACACGGAAAGCTGTAAATTTTGCTGGAACTGTCAAA
ATGGCCTTTGTGAGGGCAAATTGGAGAATACAACAATTCTTGAAAATGAGTATGTGCAATCATGTATAGTTTCCCGCTGGCTA
AATAAATGTATGTATGATCTAGGACAGGGGATTGACCATGTAATGGCCTGTTCTGAACCAAAGCCCTGGAATCCTTACAAAAT
CTTAAAGAGAGAGTGGAAAAAAAATGTCAGCCAAAATTAA
SEQ ID No. 269
Ken06.Bus 6224-5853 MGF 110 5L
ATGTTGGTGATCTTCTTGGGACTCCTTGGCCTGCTGGCCAACCAGGTCTTAGGGCTACCTAATCAACCAGCTGGACAACTTCA
TCCAACGGATAATCCTCCAAAAGAAGAACTTGGATACTGGTGCACTTACACGGAAAGCTGTAAATTTTGCTGGAACTGTCAAA
ATGGCCTTTGTGAGGGCAAATTGGAGAATACAACAATTCTTGAAAATGAGTATGTGCAATCATGTATAGTTTCCCGCTGGCTA
AATAAATGTATGTATGATCTAGGACAGGGGATTCACCATGTAATGCCCTGTTCTGAACCAAAGCCCTGGAATCCTTACAAAAT
CTTAAAGAAAGAGTGGAAAGAAAATGTCAGCCAAAATTAA
SEQ ID No. 270
Malawi Lil-20/1/1983 7878-7516 MGF 110 5L
ATGTTGGTTATCTTCTTGGGAATTCTTGGCCTTCTGGCCAACCAGGTCTCAAGCCAGCTCGTTGGACAACTTCATCCAACGGA
AAATCCTTCAGAGAATGAACTTGAATACTGGTGCACTTACATGGAATGTTGCCAGTTTTGCTGGGACTGTCAAGATGGCCTTT
GTGTGAATAAGTTGGGAAATACAACAATTCTTGAAAATGAGTATGTGCATCCATGTATAGTTTCCCGCTGGCTAAATAAATGT
ATGTATGATCTGGGTCAAGGCATTGATCATGTAATGGTCTGTTCTCAACCAAAGTACTGGAATCCTTATAAAATCTTAAAGAA
AGAGTGGAAAGAAAATAACAGCCAAAATTAA
SEQ ID No. 271
Mkuzi 1979 (genotype XII) 10334-9972 MGF 110 5L
ATGTTAGTAATCTTCTTGGGAATTCTTGGCCTTCTGGCCAACCAGGTCTCAAGCCAGCTCGTTGGACAACTTCATCCAACGGA
AAATCCTTCAGAGAATGAACTTGAATACTGGTGCACTTACATGGAATGTTGCCAGTTTTGCTGGGACTGTCAAGATGGCCTTT
GTGTGAATAAGTTGGGAAATACAACAATTCTTGAAAATGAGTATGTGCATCCATGTATAGTTTCCCGCTGGCTAAATAAATGC
ATGTATGATCTGGGTCAAGGCATTGATCATGTAATGGTCTGTTCTCAACCAAAGTACTGGAATCCTTATAAAATCTTAAAGAA
AGAGTGGAAAGAAAATAACAGTCAAAATTAA
SEQ ID No. 272
Pretorisuskop/96/4 (genotype XX/I) 9424-9059 MGF 110 5L
ATGTTAGTAATCTTCTTGGGAATTCTTGGCCTTCTGGCCAACCAGGTCTCAAGCCAGCTCGTTGGACAACTTCATCCAACGGA
AAATCCTTCAGAGAATGAACTTGAATACTGGTGCACTTACATGGAATGTTGCCAGTTTTGCTGGGACTGTCAAAATGGCCTTT
GTGTGAATAAGTTGGGAAATACAACAATTCTTGAAAATGAGTATGTGCATCCATGTATAGTTTCCCGCTGGCTAAATAAATGT
ATGTATGATCTGGGTCAAGGCATTGATCATGTAATGGTCTGTTCTCAGCCAAAATACTGGAATCCTTATAAAATCTTAAAGAA
AGAGTGGAAAGAAAATAACAGCCAAAATAAATAA
SEQ ID No. 273
Tengani62 (genotype V/I) 8803-8447 MGF 110 5L
ATGTTAGTCATCTTCTTGGGAATTCTTGGCCTTCTGGTCAACCAGGTCTCAAGCCAGCCAGTTGGACAACTTCATCCAACGGA
AAATCCTTCAGAGAATGAACTTGAATACTGGTGCACTTACATGGAATGTTGCCAGTTTTGCTGGGACTGTCAAAATGGCCTTT
GTGTGAATAAGTTGGGAAATACAACAATTCTTGAAAATGAGTATGTGCATCCATGTATAGTTTCCCGCTGGCTAAATAAATGT
ATGTATGATCTGGGTCAAGGCATTGATCATGTAATGGTCTGTTCTCAACCAAAATACTGGAATCCTTATAAAATCTTAAAGAA
AGAGTGGAAAGAAAATAATAGTTAA
SEQ ID No. 274
Warmbaths (genotype III/I) 8649-8284 MGF 110 5L
ATGTTAGTAATCTTTTTGGGAATTCTTGGCCTTCTGGCCAACCAGGTCTCAAGCCAGCTCGTTGGACAACTTCATCCAACGGA
AAATCCTTCAGAGAATGAACTTGAATACTGGTGCACTTACATGGAATGTTGCCAGTTTTGCTGGGACTGTCAAAATGGCCTTT
GTGTGAATAAGTTGGGAAATACAACAATTCTTGAAAATGAGTATGTGCATCCATGTATAGTTTCCCGCTGGCTAAATAAATGT
ATGTACGATCTGGGTCAAGGCATTGATCATGTAATGGTCTGTTCTCAACCAAAATACTGGAATCCTTATAAAATCTTAAAGAA
AGAGTGGAAAGAAAATAACAGCCAAAATAAATAA
SEQ ID No. 275
Warthog (genotype IV) 7349-6984 MGF 110 5L
ATGTTAGTAATCTTCTTGGGAATTCTTGGCCTTCTGGCCAACCAGGTCTCAAGCCAGCTCGTTGGACAACTTCATCCAACGGA
AAATCCTTCAGAGAATGAACTTGAATACTGGTGCACTTACATGGAATGTTGCCAGTTTTGCTGGGACTGTCAAAATGGCCTTT
GTGTGAATAAGTTGGGAAATACAACAATTCTTGAAAATGAGTATGTGCATCCATGTATAGTTTCCCGCTGGCTAAATAAATGT
ATGTACGATCTGGGTCAAGGCATTGATCATGTAATGGTCTGTTCTCAACCAAAATACTGGAATCCTTATAAAATCTTAAAGAA
AGAGTGGAAAGAAAATAACAGCCAAAATAAATAA

In an embodiment the attenuated ASFV of the invention comprises a functional version of MGF 110 5L. Suitably the functional version of MGF 110 5L comprises the sequence of SEQ ID No. 266, 267, 268, 269, 270, 271, 272, 273, 274 or 275. Suitably the functional version of MGF 110 5L comprises a sequence having at least 70%, at least 80%, at least 90% or at least 95% identity with SEQ ID No. 266, 267, 268, 269, 270, 271, 272, 273, 274 or 275. 40 Suitably the functional version of MGF 110 5L consists of the sequence of SEQ ID No. 266, 267, 268, 269, 270, 271, 272, 273, 274 or 275.

MGF110 6L gene sequences
SEQ ID No. 35
Ken05/Tk1 MGF 110 6L
ATGTTGGTAATCTTTTTGGGAATTCTTGGCCTTCTGGCCAGCCAGGTCTCAAGTCAACCAGATGGACAACTTCGTCCAACAGA
GGATCCTCCAGAAGAAGAACTTAAATATTGGTGCACCTACATGGAAAGTTGCCAGTTTTGTTGGGACTGCCAAGATGGCAATT
GTATAAACAAAGTAGATGGGTCAGTCATTTATAAAAATGAGTTTGTGCGACCATGTTCAGTTTCCCGCTGGATGAATAAATGT
ATGTATGATTTAAATAAGGGTATCTATCATACAATGAATTGTTCTCAGCCACAGTCTTGGAATCCCTACAAATACTTCAGGAA
GGAGTGGAAAAAAGATGAACTCTAG
SEQ ID No. 36
Ken06.Bus MGF 110 6L
ATGTTGGTGATCTTTTTGGGAATTCTTGGCCTTCTGGCCAGCCAGGTCTCAAGTCAGCCAGTTGGACAACTTCGTCCAACAGA
GGACCCTCCAGAGAATGAACTCGAATATTGGTGCACCTACATGGAAAGTTGCCAGTTTTGCTGGGACTGTCAAGATGGCAATT
GTATAAACAAAGTAGATGGGTCAGTCATTTATAAAAATGAGTATGTGCAACCATGTTCAGTTTCCCGCTGGCTAAATAAATGT
ATGTATGATTTAGATAAGGGTATCTATCATACAATGAACTGTTCTCAGCCACGGTCTTGGAATCCCTACAAATGTTTCAGGAA
GGAGTGGAAAAAAGATGAACTCTAG
SEQ ID No. 37
Kenya 1950 MGF 110 6L
ATGTTGGTGATCTTTTTGGGAATTCTTGGCCTTATGGCCAGCCAGGTCTTAGGGCTACCATCTAATCAACCAACTGGACAACT
TCGTCCAACAGAGGATCCTCCAGAGGAAGAACTCGAATACTGGTGTGCCTACATGGAAAGTTGCCAGTTTTGCTGGGACTGCC
AAGATGGCAATTGCATAAACAAAATAGATGGGTCAGTCATTTATAAAAATGAGTTTGTGCGACCATGTTCAGTTTCCCGCTGG
ATGGATAAATGTATGTATGATTTAAATAAGGGTATCTATCATACAATGAATTGTTCTCAGCCACAGTCTTGGAATCCCTACAA
ATACTTCAGGAAGGAGTGGAAAAAAGATGAACTCTAG
SEQ ID No. 38
Malawi Lil-20/1 (1983) MGF 110 6L
ATGTTGGTGACCTTTTTGGGAATTCTTGGCCTTCTGGCCAGCCAGGTCTCAAGTCAGCTCGTTGGACAACTTCGTCCAACAGA
GGATCCTCCAGAGGAAGAACTCGAATACTGGTGCGCCTACATGGAAAGTTGCCAGTTTTGCTGGGACTGCCAAGATGGCAATT
GTATAAACAAAATAGATGGGTCAGTCATTTATAAAAATGAGTATGTGCGACCATGTTCAGTTTCCCGTTCGATGGATAAATGT
ATGTATGATTTAAATAAGGGTATCTACCATAGCATGAGCTGTTCTGACCCAAAGGCCTGGAATCCCTACAAATACTTCAGGAA
GGAATGGAAAAAAGATGAACTCTAG
SEQ ID No. 39
Mkuzi 1979 MGF 110 6L
ATGTTGGTGATCTTTTTGGGAATTCTTGGCCTTCTGGCCAGCCAGGTTTCAAGTCAACTCGTTGGACAACTTCGACCAACAGA
GGATCCTCCAGAGGAAGAACTCGAATACTGGTGCGCCTACATGGAAAGTTGCCAATTTTGCTGGGACTGCCAAGATGGCACTT
GTATAAACAAAATAGATGGGTCAGTAATTTACAAGAATGAGTATGTGAAAGCATGTCTGGTTTCCCGTTGGCTGGATAAATGT
ATGTATGATTTAGATAAAGGTATCTACCATACCATGAATTGTTCTCAGCCATGGTCTTGGAATCCTTACAAATACTTCAGGAA
GGAATGGAAAAAAGATGAACTCTAG
SEQ ID No. 40
Pretorisuskop/96/4 MGF 110 6L
ATGTTGGTGATCTTCTTGGGAATTCTTGGCCTTCTGGCCAGCCAGGTTTCAAGTCAGCTCGTTGGACAACTTCGTCCAACAGA
GGATCCTCCAGAGGAAGAACTCGAATACTGGTGCGCCTACATGGAAAGTTGCCAATTTTGCTGGGACTGCCAAGATGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAAGGTATCTACCATACCATGAATTGTTCTCAGCCATGGTCTTGGAATCCTTACAAATACTTCAGGAA
GGAATGGAAAAAAGATGAACTCTAG
SEQ ID No. 41
Tengani62 MGF 110 6L
ATGTTGGTGATCTTCTTGGGAATTTTTGGCCTTCTGGCCAGCCAGGTTTCAAGTCAGCTCGTTGGACAACTTCGTCCAACAGA
GGATCCTCCAGAGGAAGAACTCGAATACTGGTGTGCCTACATGGAAAGTTGCCAATTTTGCTGGGACTGCCAAGATGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAGGGTATCTACCATACCATGAATTGTTCTCAGCCATGGTCTTGGAATCCTTACAAATACTTCAGGAA
GGAATGGAAAAAAGATGAACTCTAG
SEQ ID No. 42
Warmbaths MGF 110 6L
ATGTTGGTGATCTTCTTGGGAATTCTTGGCCTTCTGGCCAGCCAGGTTTCAAGTCAGCTCGTTGGACAACTTCGTCCAACAGA
GGATCCTCCAGAGGAAGAACTCGAATACTGGTGCGCCTACATGGAAAGTTGCCAATTTTGCTGGGACTGCCAAGATGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAAGGTATCTACCATACCATGAATTGTTCTCAGCCATGGTCTTGGAATCCTTACAAATACTTCAGGAA
GGAATGGAAAAAAGATGAACTCTAG
SEQ ID No. 43
Warthog MGF 110 6L
ATGTTGGTGATCTTCTTGGGAATTCTTGGCCTTCTGGCCAGCCAGGTTTCAAGTCAGCTCGTTGGACAACTTCGACCAACAGA
GGATCCTCCAGAGGAAGAACTCGAATACTGGTGCGCCTACATGGAAAGTTGCCAATTTTGCTGGGACTGCCAAGATGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAAGGTATCTACCATACCATGAATTGTTCTCAGCCATGGTCTTGGAATCCTTACAAATACTTCAGGAA
GGAATGGAAAAAAGATGAACTCTAG

In an embodiment the attenuated ASFV of the invention comprises a functional version of MGF 110 6L. Suitably the functional version of MGF 110 6L comprises the sequence of SEQ 5 ID No. 35, 36, 37, 38, 39, 40, 41, 42 or 43. Suitably the functional version of MGF 110 6L comprises a sequence having at least 70%, at least 80%, at least 90% or at least 95% identity with SEQ ID No. 35, 36, 37, 38, 39, 40, 41, 42 or 43. Suitably the functional version of MGF 110 6L consists of the sequence of SEQ ID No. 35, 36, 37, 38, 39, 40, 41, 42 or 43.

MGF110 7L gene sequences
China/2018/AnhuiXCGQ MGF 110 7L
SEQ ID No. 247
ATGCTGGTGATTATCCTGGGAATTATTGGCCTGCTGGCCAGTAGCAACCTGGTTTCATCCTCCACTTCTACTCGGGTAGGTGG
ACATCTTCCTCTAACATTTGAACCCCCAGAGAATGAACTTGGATACTGGTGCACTTATGTGGAAAGCTGTCGGTTCTGCTGGG
ATTGTGAAGATGGGATTTGTACCAGCAGGGTTTGGGGAAACAACTCCACAAGTATTATTGAGAATGACTATGTAAAATATTGT
GAGGTTTCCCGCTGGGGTGACCTATGTAGATATGATGTGGAGGAGCACATTTACCATAGCATGAACTGTTCTGACCCAAAGCC
CTGGAATCCTTATAAAATTGCAAGGAAGGAGTGGAAAAAGAATGAACATCCCAGAAAGGATTTGAAAAAAGATGAATTCTAG
Georgia 2007/1 MGF 110 7L
SEQ ID No. 248
ATGCTGGTGATTATCCTGGGAATTATTGGCCTGCTGGCCAGTAGCAACCTGGTTTCATCCTCCACTTCTACTCGGGTAGGTGG
ACATCTTCCTCTAACATTTGAACCCCCAGAGAATGAACTTGGATACTGGTGCACTTATGTGGAAAGCTGTCGGTTCTGCTGGG
ATTGTGAAGATGGGATTTGTACCAGCAGGGTTTGGGGAAACAACTCCACAAGTATTATTGAGAATGACTATGTAAAATATTGT
GAGGTTTCCCGCTGGGGTGACCTATGTAGATATGATGTGGAGGAGCACATTTACCATAGCATGAACTGTTCTGACCCAAAGCC
CTGGAATCCTTATAAAATTGCAAGGAAGGAGTGGAAAAAGAATGAACATCCCAGAAAGGATTTGAAAAAAGATGAATTCTAG
Ken05/Tk1 MGF 110 7L
SEQ ID No. 249
ATGCTGGTGATTATCCTGGGAGTTATTGGCCTGCTGGCCAGTAGCAACTTGGTTTCATCCTCCACTTCTACTCGGATAGGTGG
ACATCTTCCTCTAACATTTGATCCTCCAGAGAATGAACTTGGATACTGGTGCACTTATGTGGAAAGCTGTCGGTTCTGCTGGG
ATTGTGAAGATGGGGTCTGTACTAGCAGGATCTGGGGGAACAACTCCACAAGTATTGTTGAGAACAGCTATGTAAAATATTGT
GAAGTTTCCCGCTGGGGTGACCAGTGTAGATATGATGTGGAGGAGCGCATTTACCATACCATGAACTGTTCTGACCCAAAACC
CTGGAATCCTTATAAAATTACAAGGGTGGAGTGGAAAAAGAATGAACATTTCAGAAAGGATTTGAAAAAAGATGAATTCTAG
Kenya 1950 MGF 110 7L
SEQ ID No. 250
ATGCTGGTGATTATCCTGGGAGTTATTGGCCTGCTGGCCAGTAGCAACTTGGTTTCATCCTCCACTTCTACTCGGGTAGGTGG
ACATCTTCCTCTAACATTTGATCCTCCAGAGAATGAACTTGGATACTGGTGCACTTATGTGGAAAGCTGTCGGTTCTGCTGGG
ATTGTGAAGATGGGGTCTGTACTAGCAGGATCTGGGGGAACAACTCCACAAGTATTGTTGAGAACAGCTATATAAAATATTGT
GAAGTTTCCCGCTGGGGTGACCAGTGTAGATATGATGTGGAGGAGCACATTTACTATACCATGAACTGTTCTGACCCAAAGCC
CTGGAATCCTTATAAAATTGCAAGGAAGGAGTGGAAAAAGAATGAACATTTCAGAAAGGATTTGAAAAAAGATGAATTCTAG
Malawi Lil-20/1 (1983) MGF 110 7L
SEQ ID No. 251
ATGCTGGTGATTATCCTGGGAGTTATTGGCCTGCTGGCCAGTAGCAACTTGGTTTCATCCTCCACTTCTACTCGGGTAGGTGG
ACATCTTCCTCTAACATTTGATCCTCCAGAGAATGAACTTGGATACTGGTACACTTATGTGGAAAGTTGTCGGTTCTGCTGGG
ATTGTGAAGATGGGGTCTGTACTAGCAGGGTTTGGGGAAACAACTCCACAAGTATTGTTGAGAATGACTATGTAAAATATTGT
GAGGTTTCTCGCTGGGGTGACCAATGTAGATATGATGTGGAGGAGCACATTTACTATACCATGAACTGTTCTGACCCAAAGCC
CTGGAATCCTTATAAAATTGCAAAGGAAGGAGTGGAAAAAGGATGA
Mkuzi 1979 MGF 110 7L
SEQ ID No. 252
ATGCTGGTGATTATCCTGGGAGTTATTGGCCTGCTGGCCAGTAGCAACTTGGTTTCATCCTCCACTTCTACTCGGGTAGGTGG
ACATCTTCCTCTAACAGAGGATCCTCCAGAGAATGAACTTGGATACTGGTGCACTTATGTGGAAAGCTGTCGGTTCTGCTGGG
ATTGTGAAGATGGGATTTGTACTAGCAGGGTTTGGGGAAACAACTCCACAAGTATTGTTGAGAACGACTATGTAAAATATTGT
GAGGTTTCTCGCTGGGGTGACCAATGTAGATATGATGTAGAGGAGCACATTTACTATACCATGAACTGTTCTGACCCAAAGCC
CTGGAATCCTTATAAAATTGCAAGGAAGGAGTGGAAAAAGGATGAACATTCCAGAAAGAATTTGAAAAAAGATGAATTCTAG
Pretorisuskop/96/4 MGF 110 7L
SEQ ID No. 253
ATGCTGGTGATTATCCTGGGAATTATTGGCCTGCTGGCCAGTAGCAACCTGGTTTCATCCTCCACTTCTACTCGGGTAGGTGG
ACATCTTCCTCTAACATTTGATCCCCCAGAGAATGAACTTGGATACTGGTGCACTTATGTGGAAAGCTGTCGGTTCTGCTGGG
ATTGTGAAGATGGGATTTGTACCAGCAGGGTTTGGGGAAACAACTCCACAAGTATTGTTGAGAATGACTATGTAAAATATTGT
GAGGTTTCCCGCTGGGGAAACCTATGTAGATATGATGTGGAGGAGCACATTTACTATAGCATGAACTGTTCTGACCCAAAGCC
CTGGAATCCTTATAAAATTGCAAGGAAGGAGTGGAAAAAGAATGAATATCTCAGAAAGGATTTGAAAAAAGATGAATTCTAG
Tengani 62 MGF 110 7L
SEQ ID No. 254
ATGAACTGTTCTGACCCAAAGCCCTGGAATCCTTATAAAATTGCAAGGAAGGAGTGGAAAAAGAATGAACATCCCAGAAAGGA
TTTGAAAAAAGATGAATTCTAG
Warmbaths MGF 110 7L
SEQ ID No. 255
ATGCTGGTGATTATCCTGGGAATTATTGGCCTGCTGGCCAGTAGCAACCTGGTTTCATCCTCCACTTCTACTCGGGTAGGTGG
ACATCTTCCTCTAACATTTGATCCCCCAGAGAATGAACTTGGATACTGGTGCACTTATGTGGAAAGCTGTCGGTTCTGCTGGG
ATTGTGAAGATGGGATTTGTACCAGCAGGGTTTGGGGAAACAACTCCACAAGTATTGTTGAGAATGACTATGTAAAATATTGT
GAGGTTTCCCGCTGGGGTAACCTATGTAGATATGATGTGGAGGAGCACATTTACCATAGCATGAACTGCTCTGACCCAAAGCC
CTGGAATCCTTATAAAATTGCAAGGAAGGAGTGGAAAAAGAATGAACATCCCAGAAAGGATTTGAAAAAAGATGAATTCTAG
Warthog MGF 110 7L
SEQ ID No. 256
ATGCTGGTGATTATCCTGGGAATTATTGGCCTGCTGGCCAGTAGCAACCTGGTTTCATCCTCCACTTCTACTCGGGTAGGTGG
ACATCTTCCTCTAACATTTGATCCCCCCGAGAATGAACTTGGATACTGGTGCACTTATGTGGAAAGCTGTCGGTTCTGCTGGG
ATTGTGAAGATGGGATTTGTACCAGCAGGGTTTGGGGAAACAACTCCACAAGTATTGTTGAGAATGACTATGTAAAATATTGT
GAGGTTTCCCGCTGGGGTAACCTATGTAGATATGATGTGGAGGAGCACATTTACCATAGCATGAACTGCTCTGACCCAAAGCC
CTGGAATCCTTATAAAATTGCAAGGAAGGAGTGGAAAAAGAATGAACATCCCAGAAAGGATTTGAAAAAAGATGAATTCTAG

In an embodiment the attenuated ASFV of the invention comprises a functional version of MGF 110 7L. Suitably the functional version of MGF 110 7L comprises the sequence of SEQ ID No. 247, 248, 249, 250, 251, 252, 253, 254, 255 or 256. Suitably the functional version of MGF 110 7L comprises a sequence having at least 70%, at least 80%, at least 90% or at 30 least 95% identity with SEQ ID No. 247, 248, 249, 250, 251, 252, 253, 254, 255 or 256. Suitably the functional version of MGF 110 7L consists of the sequence of SEQ ID No. 247, 248, 249, 250, 251, 252, 253, 254, 255 or 256.

Ken05/Tk1 MGF 110 8L
SEQ ID No. 44
ATGAAGGTGCTGATTCTAGTACTACTGGGGGTCGTTATCCTTCAGGCTGCTCCTATACGTAAATTAGAAGACCTACTGCCAAC
CCGTAATCCTCCCCAAAATGAGCTAGTTTACTGGTGCACCTACGCAAACCAATGTGACTTTTGCTGGGAATGTGTACACGGAA
TCTGCCGAAACAGAATTCAAGCAGATTGGCCTGTGATTCACCAAAATGACTGGATTATAAATTGCACCGTTTCCCGCTGGAAT
GGTATATGTAGGTATTATGAAGGACCCAGAGTCCATATAGATCATGAAATGGACTGTGCAAATCCAACATCCCATACTTATCC
ACACATTGAATACATGAAGATCTATGAAAGAGATGACCTATGA
Kenya 1950 MGF 110 8L
SEQ ID No. 45
ATGAAGGTGCTGATTCTAGTACTACTGGGGGTCGTTATCCTTCAGGCTGCTCCTATACGTAAATTAGAAGACCTACTGCCAAC
CCGTAATCCTCCCCAAAATGAGTTAGTTTATTGGTGCACCTACGCAAACCAATGTGATTTTTGCTGGGAATGTATACACGGAA
TCTGCCGAAACAGAATTCAAGCAGATTGGCCTGTGATTCACCAAAATGACTGGATTATAAATTGCACCGTTTCCCGCTGGAAT
GGTATATGTAGGTATTATGAAGGACCCAGAGGCCATATAGATCATGAAATGGACTGTGCAAATCCAACATACCATACTTATCC
ACACATTGAATACATGAAGATTTATGTAAGAGATGACCTATGA
Malawi Lil-20/1 (1983) MGF 110 8L
SEQ ID No. 46
ATGAAGGTGCTGATTCTAGTACTGCTGGCGGTGGTTATCCTTCAAGCCGCTCCTATACGTAAATTAGAAGATCTACTACCAAC
CCGTTATCCTCCTGACCATGAGCTAGTTTATTGGTGCACCTACGCAAACCAATGTGACTTTTGCTGGGAATGCGTACACGGTA
TCTGCCGAAATAGGATTCAAGCAGATTGGCCGGTTATTCACCAAAATGACTGGATTATAAATTGCACGGTTTCCCGCTGGAAT
GGTATATGTAGTTATTATGAAGGACCCAGAAACCATACAGATCATCAAATGGACTGTGCAAATCCAACATCCCATACCTATCC
ACATAGAGAATACATGAAAATTTATGAAAGAGATGACTTATAA
Mkuzi 1979 MGF 110 8L
SEQ ID No. 47
ATGAAGGTGCTGATTCTAGTACTACTGGGGGGGGGGGGTATTATTCTTCAGGCCGCTCCTATACGTAAATTAGAAGATCTACT
ACCAACCCGTTATCCTCCTGAACATGAGCTGGTTTATTGGTGCACCTACGCAAACCAATGTGACTTTTGCTGGGAATGCGTCC
ACGGTATTTGCCGAAACAGGATTCAAGCAGATTGGCCGGTTATTCATCAAAATGACTGGATTATAAATTGCACGGTTTCCCGC
TGGAATGGCCAATGTCATTATTATGAAGGTTCTCAACAATATCTACATCATGAAATGGACTGTACAAATCCAACATCCCATAC
CTATCCACATACAGAATACATGAAGATCTATGAAAGAGATGACTTATGA
Pretorisuskop/96/4 MGF 110 8L
SEQ ID No. 48
ATGAAGGTGCTAATTCTAGTACTACTGGGGGTGGTTATCCTTCAGGCCGCTCCTATACGTAAATTAGAAGATCTACTACCAAC
CCGTTATCCTCCTGAACATGAGCTAGTTTATTGGTGCACTTACGCAAACCAATGTGACTTTTGCTGGGAATGCGTCCACGGTA
TCTGCCGAAACAGGATTCAAGCAGATTGGCCAGTTATTCATCAAAATGACTGGATTATAAATTGCACGGTTTCCCGCTGGAAT
GGTATATGTAGTTATTATGAAGGACCCAGAAACCATACAGATCATCAAATGGACTGTGCAAATCCAACATCCCATACTTATCC
ACATAGAGAATACATGAAGATCTATGAAAGAGATGACTTATGA
Warmbaths MGF 110 8L
SEQ ID No. 49
ATGAAGGTGCTAATTCTAGTACTACTGGGGGTGGTTATTCTTCAGGCCGCTCCTATACGTAAATTAGAAGATCTACTACCAAC
CCGTTATCCTCCTGAACATGAGCTAGTTTATTGGTGCACTTACGCAAACCAATGTGACTTTTGCTGGGAATGCGTCCACGGTA
TCTGCCGAAACAGGATTCAAGCAGATTGGCCAGTTATTCACCAAAATGACTGGATTATAAATTGCACAGTTTCTCGCTGGAAT
GGTATATGTAGTTATTATGAAGGACCCAGAAACCATACAGATCATCAAATGGACTGTGCAAATCCAACATCCCATACTTATCC
ACATAGAGAATACATGAAGATCTATGAAAGAGATGACTTATGA
SEQ ID No. 50
Warthog MGF 110 8L
ATGAAGGTGCTAATTCTAGTACTACTGGGGGTGGTTATTCTTCAGGCCGCTCCTATACGTAAAGTAGAAAATCTACTGCCAAC
CCGTAATCCTCCCCAAAATGAATTAGTTTATTGGTGCACCTATGCAAACCAATGTGACTTTTGCTGGGAATGCATTCACGGCA
TCTGCCGAAACAGGATTCAAGCAGATTGGCCAGTTATTCATCAAAATGACTGGATTATAAATTGCACGGTTTCCCGCTGGAAT
GGCCAATGTCATTATTATGAAGGTTCTCAACAATATCTGCATCATGAAATGGACTGTATAAATCCAACATCCCATACCTATCC
ACATAGAGAATACATGAAGATCTATGAAAGAGATGACTTATGA

In an embodiment the attenuated ASFV of the invention comprises a functional version of MGF 110 8L. Suitably the functional version of MGF 110 8L comprises the sequence of SEQ 35 ID No. 44, 45, 46, 47, 48, 49 or 50. Suitably the functional version of MGF 110 8L comprises a sequence having at least 70%, at least 80%, at least 90% or at least 95% identity with SEQ ID No. 44, 45, 46, 47, 48, 49 or 50. Suitably the functional version of MGF 110 8L consists of the sequence of SEQ ID No. 44, 45, 46, 47, 48, 49 or 50.

Benin 97/1 (genotype I) 7643-7332 MGF 110 12L
SEQ ID No. 276
ATGAAGGTTTTTCTAGGACTTTTACTAGGTTATTCAACCATCCTCATTCTTACATATCAATCACCAACAACCCAGTGGTGTTT
TTATGAAATATCACTTAAAATACTTAATCATCATAGCATGGAAAAATGGAGGGATAAGAATTGGTCAATCATTATAAGGTATT
ATTGTTTTTACCTTGTATTTAGCTTTGCATTTGCTGGTTGCGTTGCATTTGCGATCTGCAAAAATCTACGACTGTGTACAACC
ATGAAATTACTTATGCTTTTGAATATTTTGGTTTTGTTATCTCAGCCAATTTTGAATAATTGA
China/2018/AnhuiXCGQ (genotype II) 14155-13796 MGF 110 12L
SEQ ID No. 277
ATGAAGGTTTTTCTAGGACTTTTACTAGGTTATTCAACCATCCTTATTCTTACCTATCAATCACCAACAACCCCGTGGTGTTT
TTATGAAATATCACTTAAAATACCTAATCATCATAGCATGAAATGTTGTTCGTATCCTAGACTTTATGAACATGAAATGTTCA
TGGAAAAATGGAGGGATAAGAATTGGCCAATCATTATAAGGTATTATTGTTTTTACCTTGTATTTAGCTTTGTATTTGCTGGT
TGCGTTGCATTTGCGATCTGCAAAAATCTACGACTGAGTACAACCATGAAATTACTTATGCTTTTGAGTATTTTGGTTTTGTT
GTTATCTCAGCCAATCTTGAATAATTGA
Georgia 2007/1 (genotype II) 14151-13792 MGF 110 12L
SEQ ID No. 278
ATGAAGGTTTTTCTAGGACTTTTACTAGGTTATTCAACCATCCTTATTCTTACCTATCAATCACCAACAACCCCGTGGTGTTT
TTATGAAATATCACTTAAAATACCTAATCATCATAGCATGAAATGTTGTTCGTATCCTAGACTTTATGAACATGAAATGTTCA
TGGAAAAATGGAGGGATAAGAATTGGCCAATCATTATAAGGTATTATTGTTTTTACCTTGTATTTAGCTTTGTATTTGCTGGT
TGCGTTGCATTTGCGATCTGCAAAAATCTACGACTGAGTACAACCATGAAATTACTTATGCTTTTGAGTATTTTGGTTTTGTT
GTTATCTCAGCCAATCTTGAATAATTGA
Ken05/Tk1 (genotype IX) 15456-14950 MGF 110 12L
SEQ ID No. 279
ATGAAGGTTTTTCTAGGACTTTTACTAGGTTTTTCAATCATCCTCATTCTTACATATCAATCACCGACAACTCAGCATCCTCC
TAAGGAAGAGCTTGCGTACTGGTGCACTTATGCAAAATCTTGTGACTTCTGCTGGGATTGCCAAAATGACACTTGTATAAATA
AGGTAATAAATGAGTCTATTTCGATAACTTCGATTGTAAACTGTAGAGTTACTCGCGATTCCCAATCCTGTTTTTATGAAATA
TCAGTTAAAATACCTAATCATCATAGCATGGAATGTTCATATCCTAGACTTTATGAACATGAAATGTTTATGGAAAAATGGAG
GGATGAGTATTGGCCAATCATTATAAAACAGTGTTGTTTTTACCTTGTATTTAGCATTGCATTTGCTGGATGCGTTGCATTTG
CGATCTGCAAAAATCTACGACTGCGTACAACTATAAAACTACTTATTCTTTTGAGTATTTTGGTTTGGTTGTCTCAACCAGTC
TTGAATTGA
Ken06.Bus (genotype X) 11961-11446 MGF 110 12L
SEQ ID No. 280
ATGAAGGTTTTTCTGGCACTTTTACTAGGTTATTTAACTATCCTCATTCTTACATATCAAACACCAACAACCCAGCATCCTCC
TAAGGAGGAGCTTCCATATTGGTGTACGTATGTAAAGAATTGCGACCTCTGTTGGGATTGTCAAGATAGCATCTATTGGAACA
AGGTAATAAGTGAGTCTATTTCGATAAATTCGATTATAAACTGTAGAGTTACTTGCGATTCCCAATCTCAGTCCTGCTTTTAT
GAAATATTACTTAAAATACCTAATCATCATAGCATGGAATGTTCTTATCCTGGATCGTATGAAAATGAAATGTTCATGGAAAA
ATGGAGGGATGAGAATTGGTCAATCATTATAAAACATTATTGTTTTTACCTTGTATTTAGCTTTGCATTTGCTGGTTGCGTTG
CATTTGCGATCTGCAAAAATCTACGACTGAGTACAACTATGAAATTACTTATGCTTTTGAGTATTTTGGTTTGCTTATCTCAG
CCAATCTTGAATAATTGA
Kenya 1950 (genotype X) 16240-15731 MGF 110 12L
SEQ ID No. 281
ATGAAGGTTTTTCTAGGACTTTTACTAGGTTTTTCAATCATCCTCATTCTTACATATCAATCACCGACAACCCAGCATCCTCC
TAAGGAAGAGCTTGCGTACTGGTGCACTTATGCAAAATCTTGTGACTTCTGCTGGGATTGCCAAAATGACACTTGTATAAATA
AGGTAATAAATGAGTCTATTTCGATAACTTCGATTGTAAACTGTAGAGTTACTCGCGATTCCCAGTCCTGTTTTTATGATATA
TCAGTTAAAATACCTAATCATCATAGCATGGAATGTTCATATCCTAGACTTTATGAACATGAAATGTTTATGGAAAAATGGAG
GGATGAGTATTGGCCAATCATTATAAAACAGTGTTGTTTTTACCTTGTATTTAGCTTTGCATTTGCTGGTTGCGTTGCATTTG
CGATCTGCAAAAACCTACGACTGCGTACAACTATAAAACTACTTATTCTTTTGAGTATTTTGGTTTGGTTATCTCAGCCAATC
TTGAATAATTGA
L60 (genotype I) 7993-7682 MGF 110 12L
SEQ ID No. 282
ATGAAGGTTTTTCTAGGACTTTTACTAGGTTATTCAACCATCCTCATTCTTACATATCAATCACCAACAACCCAGTGGTGTTT
TTATGAAATATCACTTAAAATACTTAATCATCATAGCATGGAAAAATGGAGGGATAAGAATTGGTCAATCATTATAAGGTATT
ATTGTTTTTACCTTGTATTTAGCTTTGCATTTGCTGGTTGCGTTGCATTTGCGATCTGCAAAAATCTACGACTGTGTACAACC
ATGAAATTACTTATGCTTTTGAATATTTTGGTTTTGTTATCTCAGCCAATTTTGAATAATTGA
Malawi Lil-20/1/1983 (genotype VIII) 12584-12069 MGF 110 12L
SEQ ID No. 283
ATGAAGGTTTTTCTAGGACTTTTACTAGGTTATTCAACCATCCTCATTCTTACATATCAATCACCAACAACCCAGCATCCTCC
TAAGGAAGAGCTTGAGTATTGGTGCACTTATGCAAAAACTTGTGACTTCTGCTGGGATTGCCAAAATGACACTTGTATAAATA
AGGTAATAAATGAATCTATTTCGATGAATTCGATTGTAAACTGTAGAGTTACTCGCGATTCCCAATCCCAGTCCTGTTTTTAT
GAAATATCACTTAAAATACCTAATTATCATAGCATGGAATGTTCATATCCTAGACTTTATAAACATTTCATGTCCATGGAAAA
ATGGAGGGATGAGAATTGGCCAATCCTTATAAGACATTATTGTTTTTACCTTGTATTTAGCTTTGCATTTGCTGGTTGCGTTG
CATTTGCGATCTGCAAAAATCTACGACTGCGTACAACCATGAAATTACTTATGCTTTTGAGTATTTTGGTTTTGTTATCTCAG
CCAATCTTGAATAATTGA
Mkuzi 1979 (genotype XII) 15894-15571 MGF 110 12L
SEQ ID No. 284
ATGAACGCAACCATGAAGGTTTTTCTAGGACTTTTACTAGGTTATTCAACCATCCTCATTCTTACATATCAATCACCGACAAC
CCAGTGGTGTTTTTATGAAATATCACTTAAAATACTTAATCATCATAGCATGGAAAAATGGAGGGATAAGAATTGGTCAATCA
TTATAAGGTATTATTGTTTTTACCTTGTGTTTAGCTTTGCATTTGCCGGTTGCGTTGCATTTGCGATCTGCAAAAATCTACGA
CTGTGTACAACCATGAAATTACTTATGCTTTTGGGTATTTTGGTTTTGTTATCTCAGCCAATCTTGAATAATTGA
Warmbaths (genotype Ill/I) 14212-13859 MGF 110 12L
SEQ ID No. 285
ATGAAGGTTTTTCTAGGACTTTTACTAGGTTATTCAACCATCCTCATTCTTACATATCAATCACCAGCAACCCAGTGGTGTTT
TTATGAAATATCACTTAAAATACCTAATCATCATAGCATGGAATGTTCATATCCTAGACTTTATAAACATTTCATATTCATGG
AAAAATGGAGGGATAAGAATTGGTCAATCATTATAAGGTATTATTGTTTTTACCTTGTATTTAGTTTTGCATTTGCTGGTTGC
ATTGCATTTGCGATCTGCAAAAATCTACGACTGTGTACAACTATGAAATTACTTATGCTTTTGAGTATTTTGGTTTTGTTATC
TCAGCCAATCTTGAATAATTGA
Warthog (genotype IV) 12897-12544 MGF 110 12L
SEQ ID No. 286
ATGAAGGTTTTTCTAGGACTTTTACTAGGTTATTCAACCATCCTCATTCTTACATATCAATCACCAGCAACCCAGTGGTGTTT
TTATGAAATATCACTTAAAATACCTAATCATCATAGCATGGAATGCTCATATCCTAGACTTTATAAACATTTCATGTTCATGA
AAAAATGGAGGGATAAGAATTGGTCAATCATTATAAGGTATTATTGTTTTTACCTTGTATTTAGTTTTGCATTTGCTGGTTGC
ATTGCATTTGCGATCTGCAAAAATCTACGACTGTGTACAACTATGAAATTACTTATGCTTTTGAGTATTTTGGTTTTGTTATC
TCAGCCAATCTTGAATAATTGA

In an embodiment the attenuated ASFV of the invention comprises a functional version of MGF 110 12L. Suitably the functional version of MGF 110 8L comprises the sequence of SEQ ID No. 276, 277, 278, 279, 280, 281, 282, 283, 284, 285 or 286. Suitably the functional version of MGF 110 12L comprises a sequence having at least 70%, at least 80%, at least 30 90% or at least 95% identity with SEQ ID No. 276, 277, 278, 279, 280, 281, 282, 283, 284, 285 or 286. Suitably the functional version of MGF 110 12L consists of the sequence of SEQ ID No. 276, 277, 278, 279, 280, 281, 282, 283, 284, 285 or 286.

Georgia 2007/1 MGF 360 6L (NC_044959.1:17222-18349)
SEQ ID No. 51
TTACCGCAAATTGTCTCTCAGCGAAGAAAATGAATGAAACGTTTCTGTATATTCATAGGTTGAAATTATTTTACGCACTTCAC
TAGGTTCTAATATTTTCTTATGAAGTATTGAATGGGGGCTTAAAAGTCCTTTCTTAAAAAGAAGTTTCATCATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACCTATCAACGTCTCTAAGGCCGTATTGATAT
TTGCGCCTAAGGCAAAACAATAGTATATACCCAATTTATTTTGAGGGTACATACAAGCAAGCGACATCATGTCATTTGGATCT
AAACGTATATTTTCCTGAAAATATGCATGATGGATTTCATCAACATTACCTAAGTATACAGCCGTTTTTAAACGCCAATAATC
TAGGTGAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACGATTGTAAATAAAGTCTATCAACACGTAAAAA
TCATGGCTATCAAAACTGTCGAGAATCGAAATATTGTCATAATAAATATCTATAGCTAATAAGACCTTTTGTTGTTTAATTAG
ATCAACAAACATATTATACAACCCTACATCTAAAAATTTTGGATCAGCTCCTAGTTGAATACACAGAACTTTCGTCCTTTCCG
TCTTGGCACATATGATGCCATAATTAATGTTGGCACCCCATAAAACAAATAACTTGATTAGATCAGTCTGGTTTTTCTTCACA
GCCCTCACCAAGGCTCTGTCAAGCTCATAGCTGTCAACATCAGAACATGACATAGAGCCACTGGTTACCATTTTACATTGTTT
ACAAAAACCTATGGGTCCGTTTTCCCACCATAATCCAAGCTGCTGTAAAATAAAAATATCATCCTCATGATAATTTGAAAAAG
CCTTGTTTTCTATCAAGACTTTTTTTGTAAGAACCTGTAAAGAATTCAT
China/2018/AnhuiXCGQ MGF 360 6L (MK128995.1:17221-18348)
SEQ ID No. 52
TTACCGCAAATTGTCTCTCAGCGAAGAAAATGAATGAAACGTTTCTGTATATTCATAGGTTGAAATTATTTTACGCACTTCAC
TAGGTTCTAATATTTTCTTATGAAGTATTGAATGGGGGCTTAAAAGTCCTTTCTTAAAAAGAAGTTTCATCATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACCTATCAACGTCTCTAAGGCCGTATTGATAT
TTGCGCCTAAGGCAAAACAATAGTATATACCCAATTTATTTTGAGGGTACATACAAGCAAGCGACATCATGTCATTTGGATCT
AAACGTATATTTTCCTGAAAATATGCATGATGGATTTCATCAACATTACCTAAGTATACAGCCGTTTTTAAACGCCAATAATC
TAGGTGAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACGATTGTAAATAAAGTCTATCAACACGTAAAAA
TCATGGCTATCAAAACTGTCGAGAATCGAAATATTGTCATAATAAATATCTATAGCTAATAAGACCTTTTGTTGTTTAATTAG
ATCAACAAACATATTATACAACCCTACATCTAAAAATTTTGGATCAGCTCCTAGTTGAATACACAGAACTTTCGTCCTTTCCG
TCTTGGCACATATGATGCCATAATTAATGTTGGCACCCCATAAAACAAATAACTTGATTAGATCAGTCTGGTTTTTCTTCACA
GCCCTCACCAAGGCTCTGTCAAGCTCATAGCTGTCAACATCAGAACATGACATAGAGCCACTGGTTACCATTTTACATTGTTT
ACAAAAACCTATGGGTCCGTTTTCCCACCATAATCCAAGCTGCTGTAAAATAAAAATATCATCCTCATGATAATTTGAAAAAG
CCTTGTTTTCTATCAAGACTTTTTTTGTAAGAACCTGTAAAGAATTCAT
Warmbaths MGF 360 6L (AY261365.1:17236-18363
SEQ ID No. 53
TTACCGCAAATTGTCTCTCAGTAAAGAAAATGAATGAAACGTTTCTGTATATTCATAGGTTGAAATTATTTTACGCACTTCAC
CAGGTTCTAATATTTTTTTATGAAGTATTGAATGGGGGCTTAAAAGTCCTTTCTTAAAAAGAAGTTTCATTATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACCTATCAACGTCTCTAAGGCAGTATTGATAT
TTGCACCTAAGGCAAAACAATAGTATATACCTAATTTATTCTGAGGGTACATACAAGCAAGCGGCATCATGTCATTTGGATCT
AAACGTATATTTTCCTGAAAATATGCATGATGGATTTCATCAACATTACCTAAGTATACAGCCGTTTTTAAACGCCAATAATC
TAGGTAAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACAATTGTAAATAAAGTCTATCAACACGTAAAAA
TCATGGCTATCAAACCTGTCGAGAATCGAAATATTATCATAATAAATATCTATAGCTAATAAGACCTTTTGTTGTTTAATTAG
ATCAACAAACATATTATACAACCCTACATCTAAAAATTTTGGATCAGCTCCTAGTTGAATACACAGAGCTTTCATCCTTTCTG
TCTTGGCGCATATGATGCCATAATTAATGTTGGCACCCCATAAAACAAATAACTTGATTAGATCAGTCTGGTTTTTCTTCACA
GCCCTCACCAAGGCTCTGTCAAGCTCATAGCTGTCAACGTCAGAGCATGACATAGCGCCACTAGTTACCATTTTACATTGTTT
ACAAAAACCTATGGGACCGTTTTCCCACCATAGTCCAAGCTGCTGTAAAATAAAAATATCATCTTCATGATATTTTGAAAAAG
CCTTGTTTTCTATCAAGACTTTTTTTGTAAGAACCTGTAAAGAATTCAT
L60 MGF 360 6L (NC_044941.1:11041 -12168)
SEQ ID No. 54
TTACCGCAAATTGTCTCTCAGCAAAGAAAATGAATGAAACGTTTCCGTATATTCATAGGTTGAAATTATTTTACGCACTTCAC
TAGGTTCTAATATTTTCTTATGAAGTATTGAATGGGGGCTTAAAAGTCCTTTCTTAAAAAGAAGTTTCATTATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACCTATCAACGTCTCTAAGGCAGTATTGATAT
TTGCGCCTAAGGCAAAACAATAGTATATACCTAATTTATTCTGAGGGTACATACAAGCAAGCGACATCATGTCATTTGGATCT
AAACGTATATTTTCCTGAAAATATGCATGATGGATTTCATCAACATTACCTAAGTATACAGCCGTTTTTAAACGCCAATAATC
TAGATGAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACAATTGTAAATAAAGTCTATCAATACGTAAAAA
TCATGGCTATCAAAACTGTCGAGAATCGAAATATTATCATAATAAATATCTATAGCTAATAAGACCTTTTGTTGTTTAATTAG
ATCAACAAACATATTATACAATCCTACATCTAAAAATTCTGGATTGGCTCCTAGTTCAATACACAGGTCTTTCGTCCTTTCCG
TCTTGGCACATATGATGCCATAATTAATGTTGGCACCCCATAAAACAAACAACTTGATTAGATCAGTCTGGTTTTTCTTCACA
GCCCTTACCAAGGCTCTGTCAAGCTCATAGCTGTCAACGTCAGAGCATGACATAGAGCCACTGGTTACCATTTTACATTGTTT
ACAAAAACCTATGGGGCCATTATGCCACCACAATCCAAGCTGCTGTAAAATAAAAATATCATCCTCATGATATTCTGAAAAAG
CCTTGTTTTCTATCAAGACTTTTTTTGTAAGAACCTGTAAAGAATTCAT
Benin 97/1 MGF 360 6L (NC_044956.1:10692-11819)
SEQ ID No. 55
TTACCGCAAATTGTCTCTCAGCAAAGAAAATGAATGAAACGTTTCCGTATATTCATAGGTTGAAATTATTTTACGCACTTCAC
TAGGTTCTAATATTTTCTTATGAAGTATTGAATGGGGGCTTAAAAGTCCTTTCTTAAAAAGAAGTTTCATTATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACCTATCAACGTCTCTAAGGCAGTATTGATAT
TTGCGCCTAAGGCAAAACAATAGTATATACCTAATTTATTCTGAGGGTACATACAAGCAAGCGACATCATGTCATTTGGATCT
AAACGTATATTTTCCTGAAAATATGCATGATGGATTTCATCAACATTACCTAAGTATACAGCCGTTTTTAAACGCCAATAATC
TAGATGAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACAATTGTAAATAAAGTCTATCAATACGTAAAAA
TCATGGCTATCAAAACTGTCGAGAATCGAAATATTATCATAATAAATATCTATAGCTAATAAGACCTTTTGTTGTTTAATTAG
ATCAACAAACATATTATACAATCCTACATCTAAAAATTCTGGATTGGCTCCTAGTTCAATACACAGGTCTTTCGTCCTTTCCG
TCTTGGCACATATGATGCCATAATTAATGTTGGCACCCCATAAAACAAACAACTTGATTAGATCAGTCTGGTTTTTCTTCACA
GCCCTTACCAAGGCTCTGTCAAGCTCATAGCTGTCAACGTCAGAGCATGACATAGAGCCACTGGTTACCATTTTACATTGTTT
ACAAAAACCTATGGGGCCATTATGCCACCACAATCCAAGCTGCTGTAAAATAAAAATATCATCCTCATGATATTCTGAAAAAG
CCTTGTTTTCTATCAAGACTTTTTTTGTAAGAACCTGTAAAGAATTCAT
Pretorisuskop/96/4 MGF 360 6L (AY261363.1:17123-18250)
SEQ ID No. 56
TTACCGCAAATTGTCTCTCAGCAAAGAAAATGAATGAAACGTTTCCGTATATTCGTAGGTTGAAATTATTTTACGCACTTCAC
TAGGTTCTAATATTTTCTTATGAAGTATTGAATGGGGGCTTAAAAGTCCTTTCCTAAAAAGAAGTTTCATTATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCCATTTACTTCATGATTAATGTAACGTATCAATGTCTCTAAGGCAGTATTGATAT
TTGCGCCTAAGGCAAAACAATAGTATATACCTAATTTATTCTGAGGGTACATACAAGCAAGCGGCATCATGTCATTTGGATCT
AAACGTATATTTTCCTGAAAATATGCATGATGGATTTCATCAACATTACCTAAGTATACAGCCGTTTTTAAACGCCAATCATC
TAGGTGAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACAATTGTAAATAAAGTCTATCAACACGTAAAAA
TCATGACTATCAAACCTGTCGAGAATCGAAATATTATCATAATAAATATCTATAGCTAATAAGACCTTTTGTTGTTTAATTAG
ATCAACAAACAAATTATACAACCCTACATCTAAAAATTTTGGATCAGCTCCTAGTTGGATACATAGAACTTTCGTCCTTTCCG
TCTTGGCACATATGATGCCATAATTAATGTTGGCACCCCATAAAACAAATAACTTGATTAGATCAGTCTGGTTTTTCTTCACA
GCCCTCACCAAGGCTCTGTCAAGCTCATAGCTGTCAACGTCAGAGCATGACATAGAGCCACTGGTTACCATTTTACATTGTTT
ACAAAAACCTATGGGGCCATTATGCCACCATAGTCCAAGCTGCTGTAAAATAAAAATATCATCTTCATGATAATTTGAAAAAG
CCTTGTTTTCTATCAAGACTTTTTTTGTAAGAACCTGTAAAGAATTCAT
Mkuzi 1979 MGF 360 6L (AY261362.1:18925-20052)
SEQ ID No. 57
TTACTGCAAATTGTCTCTCAGCAAAGAAAATGAATGAAACGTTTCTGTATATTCATAGGTTGAAATTATTTTACGCACTTCAT
TAGGTTCTATTATTTTCTTATGAAGTATTGAATGGGGGCTTAAAAGTCCTTTCTTAAAAAGAAGTTTCATTATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTCCTTCATGATTAATGAAACCTATCAACGTCTCTAAGGCAGTATTGATAT
TTGCGCCTAAGGCAAAACAATAGTATATACCTAATTTATTCTGAGGGTACATACAAGCAAGCGACATCATGTCATTTGGATCT
AAACGTATATTTTCTTGAAAATATGCATGATGGATTTCATCAACATTACCTAAGTATACAGCCGTTTTTAAACGCCAATCATC
TAGGTGAGGAAATTTCTTACTAAGAAAACGAATAGGTTTTATAAGATTAAACTGTATGGCGAACTTAAACCAAAATTTTAATA
CAAGTGTATTTTTTGTCATTTCTTCTTTTTCATCTAAGTATAGGATAAAACGATTGTACATAAAGTCTATCAACACGTAAAAA
TCATGGCTATCAAAACTGTCGAGAATCGAAACATTATCATAATAAATATCTATAGCTAATAAGACCTTTTGTTGTTTAAGTAG
ATCAACAAACATATTATACAACCCTACATCTAAAAATTTTGGATCAGCTCCTAGTTGAATACACAGAACTTTCGTCCTTTCCG
TCTTGGCACATATGATGCCATAATTAATGTTGGCACCCCATAAAATAAATAACTTGATTAGATCAGTCTGGTTTTTCTTCACA
GCCCTCACCAAGGCTCTGTCAAGCTCATAGCTGTCAACGTCAGAGCATGACATAGAGCCACTGGTTACCATTTTACATTGTTT
ACAAAAACCTATGGGGCCATTATGCCACCATAGTCCAAGCTGCTGTAAAATAAAAATATCATCTTCATGATATTCTGAAAAAG
CCTTGTTTTCTATCAAGACTTTTTTTGTAAGAACCTGTAAAGAATTCAT
Ken05/Tk1 MGF 360 6L (NC_044945.1:19166-20293)
SEQ ID No. 58
TTATCGTAAATTGTCTCTTAGCGAAGAAAATGAATGAACCGTTTCCGTATATTGATAGGTTGAAATTATTTTACGCACTTCAC
TAGGTTCTAATATTTTCTTATGAAGTATTGAATGGGGGCTTAAAAGTCCTTTCTTAAAAAGTAGTTTCATTATAACATTCTTT
TCTTCTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACGTATCAACGTCTCTAAGGCAGTATTGATAT
TTGCCCCTAAGGCAAAACAATAGTATATACCTAACTTATTTTGAGGGTACATACAAGCAAGCGACATCATGTTATTTGGATCT
AAACGTATATTTTCCTGAAAATATGCATGATGGATTTCATCAACATTACCTAAGTATACAGCCGTTTTTAAACGCCAATGATC
TAGGTGAGGAAATTTCTTACTAAGAAAACGAATAGGTTTTATAAGATTAAACTCTATCGCGAACTTATACCAAAATTTTAATA
CAAGTGTATTTCTTGTCATTTCTTCTTTTTCATCTAAATTTAAGATAAAACGATTGTAAATAAAGTCTATCAACGTGTAAAAA
TCATGGTCATCAAAACTATCGAGAATCGAAATATTATCATAATAAATATCTATAGCTAATAAGACTTTTTGTTGTTTAAGTAG
ATCAACAAACATATTATACAACCCTACATCTAAAAATTTTGGATCAGCTCCTAGTTGAAGACACAGAACTTTTGTCCTTTCCG
TCTTGGCACACATAATGCCATAATTAACGTTGGCACCCCATAAAACAAATAACTTGATTAGATCAGTCTGGTTTTTCTTCACA
GCCGTCACCAAGGCTCTGTCAAGCTCATAGCTGTCAACATCAGAGCATGACATAGAGCCACTGGTTACCATTTTACATTGTTT
ACAAAAACCTATGGGGCCATCATGCCACCACAACCCTAGCTGCTGTAAAATAAAAATATCATCCTCATGATAATTTGAAAAGG
CCTTGGTTTCTATCAAGACTTTTTTTGTAAGAACCTGTAAAGAATTCAT
Malawi Lil-20/1 (1983) MGF 360 6L (AY261361.1:15478-16605)
SEQ ID No. 59
TTACCGCAAATTGTCTCTCAGCGAAGAAAATGAATGAAACGTTTCCGTATATTCATAGGTTGAAATTATTTTACGCACTTCAC
CAGGTTCTAATATTTTTTTATGAAGTATTGAATGGGAGGTTAAAAGTCCTTTCTTAAAAAAGAGTTTCATTATAACATTCTTT
TCTTCTCTAAGAAGAGTTTCTTGTATTTTTTTTATATAAGGATTGGCACCCAAACTTATACAAAAATGTACATTACTCCAAAT
ACCATAATTTGAAAAGAAAGTTATTTCCCTATCTACTTCATGATTAATGAAACGTATCAACGTCTCTAAGGCAGTATTGATAT
TTGCACCTAAGGCAAAACAATAGTATATACCTAATTTATTTTGAGGGTACATACAAGCAAGCGACATCATGTGATTTGGATCT
AAACGTATATTTTCCTGAAAATATGCATGATGGATTTCATCAACATTACCTAAGTATACAGCCATTTTTAAACGCCAATCATC
TAGGTGAGGAAATTTCTTACTAAGAAAACGAATAGGTTTTATAAGATTAAACTCTATGGCGATCTTAAACCAAAATTTTAATA
CAAGTGTATTTCTTGTCATTTCTTCTTTTTCATCTAAGTATAGGATATAACGATTGTAAATAAAGTTTATCAATGCGTAAAAA
TCATGGCTATCAAAACTGTCGAGAATGGGAATGTTTTCATAATAAATATCTATAGCTAATAAGACCTTTTGTTGTTTAAATAG
ATCAACAAACATAGTATACAACCCTACCTCTAAAAATGTTGGATCAGCTCCTAGTTGAATACACAGAACTTTCGTCCTTTCCA
TCTTGGCACATATGATGCCATAATTGATGTTGGCACCCCATAAAACAAATAACTTGATTAGATCAGTCTGATTTTTCTTTACA
GCCTTCACCAAGGCTCTGTCAAGCTCATAGCTGTCAACGTCAGAGCATGACATAGAGCCACTGGTTACCATTTTACATTGTTT
ACAAAAACCTATAGGTCCGTTTTCCCACCATAATTCTAGCTGCTGTAAAATAAAAACATCATCTTCATGATAATTTGAAAAAG
CCTTGTTTTCTATCAAGACTTTTTTTGTAAGAACCTGTAAAGAATTCAT
Kenya 1950 MGF 360 6L (AY261360.1:19341 -20462)
SEQ ID No. 60
TTACCGCACATTGTCTCTCAGCGAAGAAAATGAATGAAACGTTTCCGTATATTGATAGGTTGAAATTATTTTACGCACTTCAC
TAGGTTCTAATATTTTCTTATGAAGTATTGAATGGGGGCTTAAAAGTCCTTTCTTAAAAAGTAGTTTCATTATATCATACTTT
TCTTCTCTAAGAAGAATTTCTTGTATTTTTTTTGTATAAGGATTGGCACCCAAACTTATACAAAAATGTACATTACTCCAAAT
ACCATAATTTGAAAAGAAAGGTCTATTTAGTTCATGATTAATGAAACGTATCAACGCCTCTAAGGCAGTATTGATATTTGCCC
CTAAGGCAAAACAATAGTATATACCTAATTTATTCTGAGGATACATACAAGCAAGCGACATCATGTGATTTGGATCTAAACGT
ATATTTTCCTGAAAATATGCATGATGGATTTCATCAACATTCCCTAAGTATACAGCCGTTTTTAAACGCCAATCATCTAGGTG
AGGAAATTTCTTACCAAGAAAACGAATAGGTTTTACAAGATTAAACTCTATGGCAAACTTATACCAAAATTTTAATACAAGTG
TATTTTTTGTTATTTTTTCTTTTTCATCTAAATTTAAGATAAAACCATTGTAAATAAAGTCTATCAACACGTAAAAATCATGG
CTATCAAAACTGTCGAGAATCGAAATATTATCATAATAAATATCTATAGCTAATAAGACCTTTTGTTGTTTAATTAGATCAAC
AAACATATTATACAACCCTACATCTAAAAATTTTGGATCAGCTCCTAGTTGAATACATAGAACTTTCGTCCTTTCCGTCTTGG
CACACACGATGCCAAAATTAATTTCGGCGCCCCATAAAACAAATAACTTGATTAGATCAGTTTGGTTTTCCTTCACAGCTTTT
ACTAAGGCTCTGTCAAGCTCATAGCTGTCGACATCAGAACATGACATAGAGCCACCGGTTACCATTTTACATTGTTTACAAAA
ACCTATGGGTCCGTTTTCCCACCACAGTCCAAGCTGCTGTAAAATGAAACTATCATCTTCATGATAATTTGAAAAAGCCTTGT
TTTCTATCAAGACTTTTTTTGTAAGAACCTGTAAAGAGTTCAT
Ken06.Bus MGF 360 6L (NC_044946.1:15081-16208)
SEQ ID No. 61
TTACCGCAAATTATCTCTCAGTGAAGAAAATGAATGAAACGTTTCCGTATGTTCATAGGTTGAAATTATTTTACGCACCTCAC
TAGGTTCTAATATTTTTTTATGAAGTATTGAATGGGGGCTTAAAAGTCCTTTTTTAAAAAGTAGTTTCATTATAACATTCTTT
TCTTCTCTAAGAAGAGTTTCTTGTATTTTTTTTGTATCAGGGTTGGCACCCAAACTTATACAAAAATGTATATTACTCCAAAT
ACCATAATTTGAAAAGAAAGTTATTTCCCTATTTACTTCATGATTAATGAAACGTATCAACGTCTCTAAGGCAGTATTGATAT
TTGCGCCTAAGGCAAAACAATAGTATATACCTAATTTATTCTGAGGGTACATACAAGCAAACGACATCATGTGATTTGGATCT
AAACGTATATTTTCTTGAAAATATGCATGATGGATTTCATCAACATTCCCTAAGTATACAGCCGTTTTTAAACGCCAATCATC
TAGGTGAGGAAATTTCTTACTAAGATAACGGATAGGTTTTACAAGATTAAACTCTATGGCGAACTTAAACCAAAATTTTAATA
CAAATGTGTTTCTTGTCATTTTTTCTTTTTCATCTAAATTTAGGATAAAACGATTGTAAATAAAGTTTATCAACGCGTGAAAA
TCATGGCTATTAAAGCTGTCGAGAATCAAAATATTATCATAATAAATATCTATGGCTAATAAGACCTTTTGTTGTTTAATTAG
ATTAACAAACATATTATACAGCTTTTTATCTAAAAATTCTGGATCGGCTCCTAGTTGAATACACAGGTCTTTCGTCCTTTCCA
TCTTAGCACACATAATGCCATAATTAATGTTGGCACCCCATAAAACAAATAATATGATTAGATCAGTTTGGTTTTCCTTCACA
GCTTTTACTAAGGCCCTGTCAAGCTCATAGCTGTCAACATCAGAGCATGACATTGAGCCAATGGTTACTATTTTACATTGTTT
ACAAAAACCTATGGGGCCGTTTTCCCACCACAATCCTAGCTGCTGTAAAATAAAACTATCATCTTCATGATAATTTGAAAAGG
CCTTGTTTTCTATTAAGACTTTTTTTGTAAGAACCTGTAAAGAATTCAT

In an embodiment the attenuated ASFV of the invention comprises a functional version of MGF 360 6L. Suitably the functional version of MGF 360 6L comprises the sequence of SEQ ID No. 51, 52, 53, 54, 55, 56, 57, 58, 59, 60 or 61. Suitably the functional version of MGF 40 360 6L comprises a sequence having at least 70%, at least 80%, at least 90% or at least 95% identity with SEQ ID No. 51, 52, 53, 54, 55, 56, 57, 58, 59, 60 or 61. Suitably the functional version of MGF 360 6L consists of the sequence of SEQ ID No. 51, 52, 53, 54, 55, 56, 57, 58, 59, 60 or 61.

Georgia 2007/1 MGF 360 10L (LR743116.1:26373-27410)
SEQ ID No. 62
CTAGGCCAACATACTTTTTAATTTATAGTTTTTTAATAGATGATATATTTTGTTAGGATCTGCTTCTTTTAACGTTAATAGCG
AGGAGTCTGCACTATAAATGTCTAATGATAAATGATGAGATATCAAATAGTAATTCCGTTGCTCTGCTAGGGCCTTTGCCTCT
TCAAAGGCGTCGGCTCCCAGATCTATACAAAAGAACAAGTTATCCATATTATAAAATCGTACGCAGGCAAGCATAGCTGAATT
AATATTAGCTCCTAAGAGAAAACAATAATATATGGTTAAAAAATTGTTATCTTTTGTGCAGGCCATCCGCATCATTTCATCCA
CGTCCATGCGGATCTTTTCCTTTTCATACAAATTATGTAGGTCAAACAGCTTATTAAAACAAAGAGCACAGATTAACCACCAC
GTATTTAGATACTTAAAATGTTGGTAAACATAAGAAATGGCCTCCCTAAGATTATCCTGCAATGCCACTATAAAACAGTATAT
CGTTAACATATCACCATCCGACATATTACTTAATATGTCGGTGTCTTCTACTAACCTTTTCAACTTCCAATATATGGATGACC
TTATTTCCCTTATAATGACATAGGCTGGAAAGGGATTATCATTAAAAAGTTTAAGACATAAGATAATATTACTGCTAGTAGTG
CCAGGGTGTATTAATTTAAAGAACATGTGCATAATCTTCTTTTTATCCACGCGGTACTTGGCTCCTAATTCCCAGCAAAATTC
TCGAACAGGCGGCGTATTGGCGCAAATTAACCCATAGTTGATGTCTGCGCCCCATTCTGTAAACAGTTTTATTAACTGATAGT
TGTTTTCCTTTGTAGCCAACATTAGTGCCGTATTAAGGTCCAAGCCGTCTGCAAAGCTTGGCAGCTTTATCAGCATATGTTTG
CAATCAAGGGAAATTGGGGCCTTATACCACCATAGTCCGCAGCGTTCTAAGATAACATGGTACTCAATAGATACTTGCTGTCT
GGCTAGTACCTTTTTGGCGAAGGATTGTAAGGAAGGAAACAT
China/2018/AnhuiXCGQ MGF 360 10L (MK128995.1:25402-26439)
SEQ ID No. 63
CTAGGCCAACATACTTTTTAATTTATAGTTTTTTAATAGATGATATATTTTGCTAGGATCTGCTTCTTTTAACGTTAATAGCG
AGGAGTCTGCACTATAAATGTCTAATGATAAATGATGAGATATCAAATAGTAATTCCGTTGCTCTGCTAGGGCCTTTGCCTCT
TCAAAGGCGTCGGCTCCCAGATCTATACAAAAGAACAAGTTATCCATATTATAAAATCGTACGCAGGCAAGCATAGCTGAATT
AATATTAGCTCCTAAGAGAAAACAATAATATATGGTTAAAAAATTGTTATCTTTTGTGCAGGCCATCCGCATCATTTCATCCA
CGTCCATGCGGATCTTTTCCTTTTCATACAAATTATGTAGGTCAAACAGCTTATTAAAACAAAGAGCACAGATTAACCACCAC
GTATTTAGATACTTAAAATGTTGGTAAACATAAGAAATGGCCTCCCTAAGATTATCCTGCAATGCCACTATAAAACAGTATAT
CGTTAACATATCACCATCCGACATATTACTTAATATGTCGGTGTCTTCTACTAACCTTTTCAACTTCCAATATATGGATGACC
TTATTTCCCTTATAATGACATAGGCTGGAAAGGGATTATCATTAAAAAGTTTAAGACATAAGATAATATTACTGCTAGTAGTG
CCAGGGTGTATTAATTTAAAGAACATGTGCATAATCTTCTTTTTATCCACGCGGTACTTGGCTCCTAATTCCCAGCAAAATTC
TCGAACAGGCGGCGTATTGGCGCAAATTAACCCATAGTTGATGTCTGCGCCCCATTCTGTAAACAGTTTTATTAACTGATAGT
TGTTTTCCTTTGTAGCCAACATTAGTGCCGTATTAAGGTCCAAGCCGTCTGCAAAGCTTGGCAGCTTTATCAGCATATGTTTG
CAATCAAGGGAAATTGGGGCCTTATACCACCATAGTCCGCAGCGTTCTAAGATAACATGGTACTCAATAGATACTTGCTGTCT
GGCTAGTACCTTTTTGGCGAAGGATTGTAAGGAAGGAAACAT
Ken06.Bus MGF 360 10L (NC_044946.1:23437-24507)
SEQ ID No. 64
TTACGACGTTGTATCATTGATGTGATAATCCAAATAGGCCAACATACTTTTTGATTTATAGTTGTTTAATAGATGGTATATTT
TTTTAGGGTTCGCTTCTTTTAGCGTGAATAGAGAGGGATCTGGATGATAGATATCTAATGACAAACGTTGTGATATTAAATAA
TAGTCCCGCTGCTCTGCTAGGGCTTTTGCCTCTTCGAAGGCATTGGCTCCCAAGTCTATACAAAAGAACAGATTATCTATATT
AAAGAACTGTATGGCAGCAAGCATGGCCATATTGATATCGGCCCCTAAAATAAAACAATAATACATCGTTAAAAAGTTATTAT
CTTTCGTGCAAGCTATGCGCATCATTTCATTAATGTCTATGCGAATCTTTTCCTGCTCATAGAGATAATGGAGGTCAGACAGT
TTATTAAAATACAGAGCACAGATTAACCACCACGTATTCATGTGCTTAAAATGTTCGTAAAAATACGAGACGGCCTCCCTAAG
ATCATTCTGCAAGGCCACCATAAAACAATATATAGTTAGCATATCGCCGTCCGAAACATCACGTAGTAGGTCTTCATCTTCTA
CTAACCTCCTCAGCCTCCAATAAAGAGACGACTTTATTTCCCTTATAATCACATAGGTAGGAAAAGGATTCTCATTAAAAAGC
TTATGACATAAGATAATATATTGACTTGTTTTGTTCTCTATAAATTTAAAGAACATGTGTATCACCTTTTTTTTATCCATGCG
ATAGTTCGCTCCTAATTCCCAACAAAACTCTCGGGTAGGCTGTGTATTGGCACAAATGTATCCATAGTTGATGTTTGCACCCC
ACTCCGTGAATAGTTTTATTAATTGATAGTTGTTTTCCTTTGCAGCTATCATCAATGCCGTATTGAGATCCAGGCCATCCGCA
AAGTATGGCAACCTTATTAACATGTGTTTGCAATCAAGTGAAATTGGAGCCGTATACCACCATAACCCGCAGTATTTCAAAAT
ATGATAGTAGTCTTCAGGCAGCACATGCTGACTGGCTAATATCTTTTTGGCGAAAGACTGTAAAGAAGGAAACAT
Ken05/Tk1 MGF 360 10L (NC_044945.1:27310-28380)
SEQ ID No. 65
TTACGACGTTGTATCATTATTGTGATAATCCAAATAGGCCGACATACTTTTTGATTTATAATTGTTTAATAGATGGTATATTT
TTTTAGGGTTCGCTTCTTTTAGCGTGAATAGAGAGGGATCTGGATGATAGATATCTAATGACAAACGATGTGATATTAAATAA
TAGTCCCGCTGCTCTGCTAGGGCTTTTGCCTCTTCGAAGGCATTGGCCCCCAAGTCTATACAAAAGAACAGATTATCTATATT
AAAAAACTGTATGGCAGCAAGCATCGCCATATTGATATCGGCCCCTAAAATAAAACAATAATACATCGTTAAAAAGTTGTTAT
CTTTCGTGCAAGCTATGCGCATCATTTCATTAATGTCTATGCGAATTTTTTCCTGCTCATAGAGATAATGGAGGTCAAACAGT
TTATTAAAACACAGAGCACAGATTAACCACCACGTATTCAGGTGCTTAAAATGTTGGTAAAAATACGAGACGGCCTCCCTAAG
ACCATTCTGCAAGGCCACCATAAAACAATATAAAGTTAGCATATCGCCGTCCGAAACATCGCGTAGTAGGTCTTCGTCTTCTA
CTAACCTCCTCAGCCTCCAATAAAGAGACGACTTTATTTCCCTTATAATCACATAGGTAGGAAAAGGATTCTCATTAAAAAGC
TTATGACATAAGATAATATATTGACTCGTTTTGTTCTCTATAAATTTAAAAAACATATGCATCACCTTTTTTTTATCTAAGCG
ATAGTTCGCTCCTAATTCCCAACAAAACTCTCGAGTAGGCTGTGTATTGGCACAAATGTATCCATAGTTGATGTTTGCACCCC
ACTCCGTGAACAGTTTTATTAATTGATAGTTGTTTTCCTTTGCAGCTATCATCAATGCCGTATTGAGGTCCAGGCCATCCGCA
AAGTATGGCAACCTTATTAACATGTGTTTACAATCAAGTGAAATTGGAGCCTTATACCACCATAACCCGCAGCGCTTCAAAAT
ATGATAGTAGTCTTCAGGCAGCACATGCTGGCTGGCTAATATCTTTTTGGCGAAAGATTGCAGGGAAGGAAACAT
Kenya 1950 MGF 360 10L (AY261360.1:29558-30628)
SEQ ID No. 66
TTACGACGTTGTATCATTAATGTGATAATCCAAATAGGCCGACATACTTTTTGATTTATAGTTGTTTAATAGATGGTATATTT
TTTTAGGGTTCGCTTCTTTTAGCGTGAATAGAGAGGGATCTGGATGATAGATATCTAATGACAAACGATGTGATATTAAATAA
TAGTCCCGCTGCTCTGCTAGGGCTTTTGCCTCTTCGAAGGCATTGGCCCCCAAGTCTATACAAAAGAACAGATTATCCATATT
AAAGAACTGTATGGCAGCAAGCATGGCCATATTGATATCGGCCCCTAAAATAAAACAATAATACATCGTTAAAAAGTTGTTAT
CTTTCGTGCAAGCTATGCGCATCATTTCATTAATGTCTATGCGAATTTTTTCCTGCTCATAGAGATAATGGAGGTCAAACAGT
TTATTAAAACACAGAGCACAGATTAACCACCACGTATTCAGGTGCTTAAAATGTTGGTAAAAATACGAGACGGCCTCCCTAAG
ATCATTCTGCAAGGCCACCATAAAACAATATAAAGTTAGCATATCGTCGTCCGAAACATCGCGTAGTAGGTCTTCGTCTTCTA
CTAATCTCCTCAGCCTCCAATAAAGAGACGACTTTATTTCCCTTATAATCACATAGGTAGGAAAAGGATTCTCATTAAAAAGC
TTATGACATAAGATAATATACTGACTCGTTTTGTTCTCTATAAATTTAAAAAACATATGCATCACCTTTTTTTTATCTAAGCG
ATAGTTTGCTCCTAATTCCCAACAAAACTCTCGAGTAGGCTGTGTATTGGCACAAATGTATCCATAGTTGATGTTTGCACCCC
ACTCCGTGAACAGTTTTATTAATTGATAGTTGTTTTCCTTTGCAGCTATCATCAATGCCGTATTGAGGTCCAGGCCATCCGCA
AAGTATGGCAACCTTATTAACATGTGTTTACAATCAAGTGAAATTGGAGCCTTATACCACCATAACCCGCAGCGCTTCAAAAT
ATGATAGTAGTCTTCAGGCAGCACATGCTGGCTGGCTAATATCTTTTTGGCGAAAGATTGCAGGGAAGGAAACAT
L60 MGF 360 10L (NC_044941.1:19956-21026)
SEQ ID No. 67
TTATAACGATGTATCATTGATGTCATAATTCAAATAGGCCAACATACTTTTTGATTTATAGTTTTTTAATAGACGATATATTT
TGTTAGGATCTGCTTCTTTTAACGTTAATAGCGAGGAGTCTGGACTATAAATGTCTAATGATAAACGATGAGATATCAAATAG
TAATTCTGTTGCTCTGCCAAGGCTTTTGCCTCTTCAAAGGCATCGGCCCCCAGATCTATACAAAAGAACAGGTTATCCATATT
ATAGAATCGTATGGAGGCAATCATGGCCAAATTAATATTAGCTCCTAAGATAAAACAATAATATATAGTTAAAAAATTGTTAT
CTTTTGTGCAGGCTATCCGCATCATTTCATCCATGTCCATACGGATTTTTTCTTTTTCGTACAAATTATGTAGGTCAAACAGC
TTATTATAGCAAAGAGCACATGTTAACCACCACGTATTCAGATACTTAAAATGTTGGTAAACATAAGAAATGGCCTCCCTAAG
ATTATCCTGCAATGCCACGATAAAACAGTATATAGTTAACATATCACCATCCGAAATATTACTTAATACGTTGGTATCTTCTG
CTAAATTTTTTAGCTTCCAATGTATACACGACTTTATTTCCCTTATAATGACATAGGCTGAAAAGGGATTGTCATTAAAAAAT
TTAAGACATAAGATAATATTATTACTAGTAGTGTTAGGATGTATTAATTTAAAGAATATGTGCATAATCTTCTTTTTATCCAC
TTGGTACTTGGCTCCTAATTCCCAGCAAAATTCTCGAATAGGTGGCGTATTCGCGCAAATTAACCCATAGTTGATGTCTGCGC
CCCAATCCGTAAACATTTTTATTAATTGATAGTTGTTTTCCTTTGTAGCCAACATTAGTGCCGTATTAAGGTCCAAGCCATCT
GCAAAGTTTGGCAGCTTTATCAGCATATGTTTGCAATCAAGTGAAATTGGAGCCTTATACCACCATAGCCCGCAGCGTTCTAA
GATAACATGGTAATCAATAGATACATGCTGACTGGCTAATACCTTTTTGGCGAAGGATTGCAAGGAAGGAACCAT
Benin 97/1 MGF 360 10L (NC_044956.1:19607-20677)
SEQ ID No. 68
TTATAACGATGTATCATTGATGTCATAATTCAAATAGGCCAACATACTTTTTGATTTATAGTTTTTTAATAGACGATATATTT
TGTTAGGATCTGCTTCTTTTAACGTTAATAGCGAGGAGTCTGGACTATAAATGTCTAATGATAAACGATGAGATATCAAATAG
TAATTCTGTTGCTCTGCCAAGGCTTTTGCCTCTTCAAAGGCATCGGCCCCCAGATCTATACAAAAGAACAGGTTATCCATATT
ATAGAATCGTATGGAGGCAATCATGGCCAAATTAATATTAGCTCCTAAGATAAAACAATAATATATAGTTAAAAAATTGTTAT
CTTTTGTGCAGGCTATCCGCATCATTTCATCCATGTCCATACGGATTTTTTCTTTTTCGTACAAATTATGTAGGTCAAACAGC
TTATTATAGCAAAGAGCACATGTTAACCACCACGTATTCAGATACTTAAAATGTTGGTAAACATAAGAAATGGCCTCCCTAAG
ATTATCCTGCAATGCCACGATAAAACAGTATATAGTTAACATATCACCATCCGAAATATTACTTAATACGTTGGTATCTTCTG
CTAAATTTTTTAGCTTCCAATGTATACACGACTTTATTTCCCTTATAATGACATAGGCTGAAAAGGGATTGTCATTAAAAAAT
TTAAGACATAAGATAATATTATTACTAGTAGTGTTAGGATGTATTAATTTAAAGAATATGTGCATAATCTTCTTTTTATCCAC
TTGGTACTTGGCTCCTAATTCCCAGCAAAATTCTCGAATAGGTGGCGTATTCGCGCAAATTAACCCATAGTTGATGTCTGCGC
CCCAATCCGTAAACATTTTTATTAATTGATAGTTGTTTTCCTTTGTAGCCAACATTAGTGCCGTATTAAGGTCCAAGCCATCT
GCAAAGTTTGGCAGCTTTATCAGCATATGTTTGCAATCAAGTGAAATTGGAGCCTTATACCACCATAGCCCGCAGCGTTCTAA
GATAACATGGTAATCAATAGATACATGCTGACTGGCTAATACCTTTTTGGCGAAGGATTGCAAGGAAGGAACCAT
Mkuzi 1979 MGF 360 10L (AY261362.1:27069-28139)
SEQ ID No. 69
TTATAACGATGTATCATTGATGTCATAATTCAAATAGGCCAACATACTTTTTGATTTATAGTTTTTTAATAGACGATATATTT
TGTTAGGATCTGCTTCTTTTAACGTTAATAGCGAGGAGTCTGGACTATAAATGTCTAATGATAAACGATGAGATATCAAATAG
TAATTCTGTTGCTCTGCCAAGGCCTTTGCCTCTTCAAAGGCATCGGCCCCCAGATCTATACAAAAGAACAGGTTATCCATATT
ATAGAATCGTATGGAGGCAATCATGGCCAAATTAATATTAGCTCCTAAGATAAAACAATAATATATAGTTAAAAAATTGTTAT
CTTTTGTGCAGGCTATCCGCATCATTTCATCCATGTCCATACGGATTTTTTCTTTTTCGTACAAATTATGTAGGTCAAACAGC
TTATTATAGCAAAGAGCACATGTTAACCACCACGTATTCAAATACTTAAAATGTTGGTAAACATAAGAAATGGCCTCCCTAAG
ATTATCCTGCAATGCCACGATAAAACAGTATATAGTTAACATATCACCATCCGAAATATTACTTAATACGTTGGTATCTTCTG
CTAAATTTTTTAGCTTCCAATGTATACACGACTTTATTTCCCTTATAATGACATAGGCTGAAAAGGGATTGTCATTAAAAAAT
TTAAGACATAAGATAATATTACTACTAGTAGTGCTAGGATGTATTAATTTAAAGAATATGTGCATAATCTTCTTTTTATCCAC
TTGGTGCTTGGCTCCTAATTCCCAGCAAAATTCTCGAATAGGTGGCGTATTCGCGCAAATTAACCCATAGTTGATGTCTGCGC
CCCAATCCGTAAACATTTTTATTAATTGATAGTTGTTTTCCTTTGTAGCCAACATTAGTGCCGTATTAAGGTCCAAGCCATCT
GCAAAGTTTGGCAGCTTTATCAGCATATGTTTGCAATCAAGTGAAATTGGAGCCTTATACCACCATAGCCCGCAGCGTTCTAA
GATAACATGGTGATCAATAGATACATGCTGACTGGCTAATACCTTTTTGGCGAAGGATTGCAAGGAAGGAAACAT
Pretorisuskop/96/4 MGF 360 10L (AY261363.1:26116-27186)
SEQ ID No. 70
TTATAACGTTGTATTATTGACGTCATAATTCAAATAGGCCAATATACTTTTTGATTTATAGTTTTTTAATAGATGATATATTT
TGTTAGGATCCGCTTCTTTTAACGTTAATAACGAGGAATCTGGACTATAAATGTCTAATGATAAACAATGAGATATCAAAAAG
TAATTCCGTTGCTCTGCCAGGGCTTTTGCCTCCTCAAAGGCATCGGCCCCCAGGTCTATACAAAAGAATAAGTTATCTATGTT
ATAGAATTGTATGGAGGCAATCATAGCTAAATTAATATTAGCTCCTAAGATAAAACAATAATATATGGTTAAAAAATTGTTAT
CTTTTGTGCAGGCTATCCGCATCATTTCATCCATGTCCATGCGGATCTTTTCCTTTTCATACAAATGATGTAGGTCAAACACC
TTATTATAGCAAAGAACACATGTTAACCACCACGTATTCAGATACTTAAAATGTTGGTAAACATAAGAAATGGCCTCCCTAAG
ATTATCCTGCAATGCCACGATAAAACAGTATATAGTTAACATATCTCCATCCGAAATATTACTTAATACGTTGGTATCTTCTG
CTAACTTTTTTAGCTTCCAATGTATACACGACTTTATTTCCCTTATAATGACATAGGCTGAAAAGGGATTGTCATTAAAAAGT
TTAAGACATAAGATAATATTACTACTAGTAGTGCTAGGATGTATTAATTTAAAGAATATGTGCATAATCTTCTTTTTATCCAC
TTGGTACTTGGCTCCTAATTCCCAGCAAAATTCTCGAACAGGTGGCGTATTCGCGCAAATTAACCCATAGTTGATGTCTGCGC
CCCAATCCGTAAACATTTTTATTAATTGATAGTTGTTTTCCTTTGTAGCCAACATTAGTGCCGTATTAAGGTCCAAGCCATCT
GCAAAGTTTGGCAGCTTTATCAGCATATGTTTGCAATCAAGTGAAATTGGAGCCTTATACCACCATAGCCCGCAGCGTTCTAA
GATAATATGGTGATCAATGGATACATGCTGTCTGGCTAGTACCTTTTTGGCGAAGGATTGTAAGGAAGGAAACAT
Tengani 62 MGF 360 10L (AY261364.1:20530-21600)
SEQ ID No. 71
TTATAACAATGTATCATTGATATCATCATTCAAATAGGCCAACATATTTTTTGATTTATAGTTTTTTAATAGATGATTTATTT
TGTTAGGATCTGTTTCTTTTAACGTTAATAGCAAGGAGTCTGGCTTATAAATGTCTAATGATAAACGATGAGATATTAAATAG
TAATTCCGTTGCTCTGCCAGGGCTTTTGCCTCTTCAAAGGCGTCGGCTCCCAGATCTATACAAAAGAACAAGTTATCCATATT
ATAAAATCGTACGCAGGCAAGCATAGCTGAATTAATATTAGCTCCTAAGAGAAAACAATAATATATAGTTAAAAAATTGTTAT
CTTTTGTGCAGGCCATCCGCATCATTTCATCCACGTCCATGCGGATCTTTTCCTTTTCATACAAATTATGTAGGTCAAACAGC
TTATTAAAACAAAGAGCACAGATTAACCACCACGTATTTAGATACTTAAAATGTTGGTAAACATAAGAAATGGCCTCCCTAAG
ATTATCCTGCAATGCCACTATAAAACAGTATATCATTAACATATCACCATCCGACATATTACTTAATATGTCGGTGTCTTCTA
CTAACCTTTTCAGCTTCCAATATATGGATATCCTTATTTCCCTTATAATGACATAGGCTGGAAAGGGATTATCATTAAAAAGT
TTAAGACATAAGATAATATTACTGCTAGTAGTGCCAGGATGTATTAATTTAAAGAACATGTGTATAATCTTCTTTTTATCCAC
GCGGTGCTTGGCTCCTAATTCCCAGCAAAATTCTCGAACAGACGGCGTATTGGCGCAAATTAACCCATAGTTGATGTCTGCGC
CCCATTCCGTAAACAGTTTTATTAACTGATAGTTATTTTCCTTTGTAGCCAACATTAGTGCCGTATTAAGGTCCAAGCCGTCT
GCAAAGCTTGGCAGCTTTATCAGCATATGTTTGCAGTCAAGTGAAATTGGAGCCTTATACCACCATAGTCCGCAACGTTCTAA
GATAACATGGTACTCAATAGATACTTGCTGTCTGGCTAGTACCTTTTTGGCGAAGGATTGTAAGGAAGGAAACAT
Warthog MGF 360 10L (AY261366.1:22069-23130)
SEQ ID No. 72
TCATATTGTATCATCATGGTTAAAATATGCCGTCATATTTTTTGATTTATAGTTTTTTAATAGATGATATATTTTGTTAGGGT
CCGCTTCTTTTAACGTTAATAGCGAGGAGTCTGGACTATAAATGTCTAATGATAAACGATGAGATATCAAAAAGTAATTCCGT
TGCTCTGCCAGGGCTTTTGCCTCTTCAAAGGCATCGGCCCCCAAGTCTATACAAAAGAACAGGTTATCCATATTATAAAATCG
TATGGAGGCAATCATGGCCAAATTAATATTAGCTCCTAAGATAAAACAATAATATATAGTTAAAAAATTGTTATCTTTTGTGC
AGGCTATCCGCATCATTTCATCCATGTCCATGCGGATTTTTTCCTTTTCGTACAAATTATGTAGGTCAAACAGCTTATTAAAA
CAAAGAGCACAGATTAGCCACCACGTATTCAGATATTTAAAATGTTGGTAAACATAAGAAATGGCCTCCCTAAGATTATCCTG
CAATGCCACCATAAAACAGTATATCGTTAACATATCACCATCCGAAATATTACTTAATACGTCGGTGTCTTCTACTAACTTTT
TCAGCTTCCAATATATGGATGACTTTATTTCCCTTATAATGACATAGGCTGAAAAGGGGTTATCATTAAAAAGTTTAAGACAT
AAGATAATATTACTGCTAGTAGTGCCAGGATGTATTAATTTAAAGAACATGTGCATAATCTTCTTTTTATCCACGCGGTACTT
GGCTCCTAGTTCCCAGCACAATTCTCGAACAGGCGGCGTATTGGCGCAAATTAACCCATAGTTGATGTCTGCGCCCCATTCCG
TAAACAGTTTTATTAACTGATAGTTGTTTTCCTTTGTAGCCAACATTAGTGCCGTATTAAGGTCCAAGCCATCTGCAAAGCTT
GGCAGCTTTATCAGCATATGTTTGCAATCAAGGGAAATTGGAGCCTTATACCACCATAGTCCGCAGCGTTCTAAGATAACATG
GTAATCAATAGATACATGCTGTCTGGCTAGTACCTTTTTGGCGAAGGATTGTAA
GGAAGGAAACAT
Warmbaths MGF 360 10L (AY261365.1:25300-26361)
SEQ ID No. 73
TCATATTGTATCATCATGGTTAAAATATGCCGTCATATTTTTTGATTTATAGTTTTTTAATAGATGATATATTTTGTTAGGGT
CCGCTTCTTTTAACGTTAATAGCGAGGAGTCTGGACTATAAATGTCTAATGATAAACGATGAGATATCAAAAAGTAATTCCGT
TGCTCTGCCAGAGCCTTTGCCTCTTCAAAGGCGTCGGCCCCCAGGTCTATACAAAAGAACAGGTTATCCATATTATAGAATCG
TATGGAGGCAATCATGGCCAAATTAATATTAGCTCCTAAGATAAAACAATAATATATAGTTAAAAAATTGTTATCTTTTGTGC
AGGCTATCCGCATCATTTCATCCATGTCCATGCGGATTTTTTCCTTTTCGTACAAATTATGTAGGTCAAACAGCTTATTAAAA
CAAAGAGCACAGATTAGCCACCACATATTCAGATACTTAAAATGTTGGTAAACATAAGAAATGGCCTCCCTAAGATTATCCTG
CAATGCCACCATAAAACAGTATATCGTTAACATATCACCATCTGAAATATCACTTAATACGTCGGTGTCTTCTACTAACTTTC
TCAGCTTCCAATATATGGATGACTTTATTTCCCTTATAATGACATAGGCTGAAAAGGGGTTATCATTAAAAAGTTTAAGACAT
AAGATAATATTACTGCTAGTAGTGCCAGGATGTATTAATTTAAAGAACATGTGCATAATCTTCTTTTTATCCACGCGGTACTT
GGCTCCTAGTTCCCAGCACAATTCTCGAACAGGCGGTGTATTGGCGCAAATTAACCCATAGTTGATGTCTGCGCCCCATTCCG
TAAACAGTTTTATTAACTGATAGTTGTTTTCCTTTGTAGCCAACATTAGTGCCGTATTAAGGTCCAAGCCATCTGCAAAGCTT
GGCAGCTTTATCAGCATATGTTTGCAATCAAGTGAAATTGGAGCCTTATACCACCATAGTCCGCAGCGTTCTAAGATAACATG
GTAATCAATAGATACATGCTGTCTGGCTAGTACCTTTTTGGCGAAGGATTGTAA
SEQ ID No. 74
GGAAGGAAACAT
Malawi Lil-20/1 (1983) MGF 360 10L (AY261361.1:23735-24802)
TTACACTGTGTCATGCCCATCATAGTCAAAATATACCATCATATTTTTTGATTTATAGTTTTTTAATAGATGATATATTTTTT
TAGGATCTGCTTCTTTTAACGTTAATAGCGAGGAGTCTGGACTATAAATATCTATTGATAAACGATGATACATCAAAAAGTAA
TTCCGCTGGTCTGCCAGGGCTTTTGCCTCTTCAAAGGCATCGGCTCCCAGGTCTATACAAAAGAACAAGTTATCCATATTATA
GAATTGTACGCAGGCAAGCATAGCCTGATTAATATTAGCTCCTAAGAGAAAACAGTAATATATGGTTAAAAAGTTGTTATCTT
TAGTGCAGGCTATGCACATCATTTCATCCATGTCCATGCGGATCTTTTCCTTTTCATACAAATCATGTAGGTCAAACAGCTTA
TTAAAACAAAGAGCACAGATTAACCACCACGTATTCAGATGCTTAAAATGTTGGTAAAAATAAGAAATGGCCTCCCTAAGATT
ATCCTGCAATGCCAGGATAAAACAGTATATAGTCAACATATCATCATCCGACATATTACTTAATATGTCAGTGTCTTCTACTA
ACCTTCTCAGCTTCCAATATATAGACGACTTTATTTCCCTTATAATGACATAGGTTGGAAAAGGATTATTATTAAAAAGTTTA
AGACATAAGATAATATTACTACTAGTAGTGCCATGATGTATTAATTTAAAAAACATATGCATAACCTTATTTTTATCCACTTG
GTACTTGGCTCCTAATTCCCAGCAAAATTCTCGAACAGGCGGCGTATTAGCGCAAATTAATCCATAGTTGATGTCCGCGCCCC
ATTCCGTAAACAGTTTTATTAACTGATAGTTGTTTTCCTTTGTAGCCAACATTAGTGCCGTATTAAGGTCCAAGCCATCTGCA
AAGCTTGGCAGCTTTATTAACATATGTTTGCAATCAAGTGAAATTGGAGCCTTATACCACCATAGTCCGCAATGTTTTAAGAT
ATAATGATAATCAATGGATACATGATGTCTAGCTAATACCTTTTTGGCGAAGGATTGTAAAGAAGGAAACAT

In an embodiment the attenuated ASFV of the invention comprises a functional version of 5 MGF 360 10L. Suitably the functional version of MGF 360 10L comprises the sequence of SEQ ID No. 62, 63, 64, 65, 66, 67, 68, 69, 70, 71, 72, 73 or 74. Suitably the functional version of MGF 360 10L comprises a sequence having at least 70%, at least 80%, at least 90% or at least 95% identity with SEQ ID No. 62, 63, 64, 65, 66, 67, 68, 69, 70, 71, 72, 73 or 74. Suitably the functional version of MGF 360 10L consists of the sequence of SEQ ID No. 10 62, 63, 64, 65, 66, 67, 68, 69, 70, 71, 72, 73 or 74.

Georgia 2007/1 MGF 360 11L (NC_044959.1:26465-27526)
SEQ ID No. 75
TTATCTTTGTTCATAATCAAGAAAAATCCCCATATTTATTGAGTGATAATTTTTTAACATGCAATTTATTTTTTCAGGGTCCG
TAACGATCGACAACAGAGAAATAACCGGATTGTAATGCTTTAATGATAAGGCATGGGCTATCAGATAATTTTCCTTTTGTTCT
GCCAAAGCTTTGCCCTCCTCAAAGGCATCGGCACCCAGGTCTATACAAAAGAACAGGTTTCCAAGATTATAGTTTTGTATGGA
AACAAGCATGGCTTGATTGATGTTGGCTCCCATGATAAAACAGTAGTAAATGGCCGAATAGCTATAATCTTGGATGCAGGCTA
TGTGCATCATTTCATCAATATCCATGCGGACCCTTTCTATTTCGTACAGCTCGTGAAGGTCGAACACGTTGTTGTAAAAAAGG
GCGCACATGAGCCGCCACCTATGTAGACGCGGGTATTTCTGGTAAAAGTAGCGGATAGCATCTTTGAGGTCATAGTCCACCGC
TATCGCGTACCAGTATTTGGTTAAAACAGTGCTAAAGCTATCATCATGGTCCAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGCTACTGGTTTTATCGCGCGTTGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAACAGGTTGAC
CTTATAAATAATTTATTGCGTCGAATATGAAGCATAATGGGGCCATTATGCCACCACAGGCCACAACACTTCAGGACATGATA
TTGATCTACCGGTATACACTGCCCGGCCAGTACTTTCTTCGTGAGGGATTGCAGGGAAGGCAACAT
China/2018/AnhuiXCGQ MGF 360 11L (MK128995.1:26467-27528)
SEQ ID No. 76
TTATCTTTGTTCATAATCAAGAAAAATCCCCATATTTATTGAGTGATAATTTTTTAACATGCAATTTATTTTTTCAGGGTCCG
TAACGATCGACAACAGAGAAATAACCGGATTGTAATGCTTTAATGATAAGGCATGGGCTATCAGATAATTTTCCTTTTGTTCT
GCCAAAGCTTTGCCCTCCTCAAAGGCATCGGCACCCAGGTCTATACAAAAGAACAGGTTTCCAAGATTATAGTTTTGTATGGA
AACAAGCATGGCTTGATTGATGTTGGCTCCCATGATAAAACAGTAGTAAATGGCCGAATAGCTATAATCTTGGATGCAGGCTA
TGTGCATCATTTCATCAATATCCATGCGGACCCTTTCTATTTCGTACAGCTCGTGAAGGTCGAACACGTTGTTGTAAAAAAGG
GCGCACATGAGCCGCCACCTATGTAGACGCGGGTATTTCTGGTAAAAGTAGCGGATAGCATCTTTGAGGTCATAGTCCACCGC
TATCGCGTACCAGTATTTGGTTAAAACAGTGCTAAAGCTATCATCATGGTCCAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGCTACTGGTTTTATCGCGCGTTGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAACAGGTTGAC
CTTATAAATAATTTATTGCGTCGAATATGAAGCATAATGGGGCCATTATGCCACCACAGGCCACAACACTTCAGGACATGATA
TTGATCTACCGGTATACACTGCCCGGCCAGTACTTTCTTCGTGAGGGATTGCAGGGAAGGCAACAT
L60 MGF 360 11L(NC_044941.1:21052-22113)
SEQ ID No. 77
TTATCTTTGTTCATAATCAAGAAAAATCCCCATATTTATTGAGTGATAATTTTTTAACATGTAATTTATTTTTTCAGGGTCCA
TAACGTTCGACAACAGAGAAATGACTGGATTGTAATGCTTTAATGATAAGGCATGGGCTATCAGATAATTTTCCTTTTGTTCT
GCCAAAGCTTTGCCCTCCTCAAAGGCGTTGGCGCCCAGGTCTATACAAAAGAACAGGTTTCCAAGATTATAGTTTTGTATGGA
AACCAGCATGGCTTGATTGATGTTGGCTCCCATGATAAAACAGTAGTAAATGGCCGAATAGCTATAATCTTGGATGCAGGCTA
TGTGCATCATTTCATCGATATCCATGCGGACCCTTTCTATTTCATACAGCTCGTGAAGGTCGAACACGTTGTTGTAAAAAAGG
GCGCACATGAGCCGCCACCGATGTAGGCGCGGGTATTTCTGGTAAAAGTAGCGAATAGCATCTTTGAGGTCATAGTCCACCGC
TATAGCATACCAGTATTTGGTTAAAACAGTGCTAAAGCTATCATCATGGTCTAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGCTACTGGTTTTATCGCGCGTCGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATAGTTTATTACGTCGAATATGAAGCATAATGGGGCCATTATGCCACCACAGGCCACAACACTTCAGGATATGATA
ATGATCCACCGACACGCACTGCCCGGCCAGTACTTTCTTCGTGAGGGATTGCAGGGAAGGCAACAT
Benin 97/1 MGF 360 11L (NC_044956.1:20703-21764)
SEQ ID No. 78
TTATCTTTGTTCATAATCAAGAAAAATCCCCATATTTATTGAGTGATAATTTTTTAACATGTAATTTATTTTTTCAGGGTCCA
TAACGTTCGACAACAGAGAAATGACTGGATTGTAATGCTTTAATGATAAGGCATGGGCTATCAGATAATTTTCCTTTTGTTCT
GCCAAAGCTTTGCCCTCCTCAAAGGCGTTGGCGCCCAGGTCTATACAAAAGAACAGGTTTCCAAGATTATAGTTTTGTATGGA
AACCAGCATGGCTTGATTGATGTTGGCTCCCATGATAAAACAGTAGTAAATGGCCGAATAGCTATAATCTTGGATGCAGGCTA
TGTGCATCATTTCATCGATATCCATGCGGACCCTTTCTATTTCATACAGCTCGTGAAGGTCGAACACGTTGTTGTAAAAAAGG
GCGCACATGAGCCGCCACCGATGTAGGCGCGGGTATTTCTGGTAAAAGTAGCGAATAGCATCTTTGAGGTCATAGTCCACCGC
TATAGCATACCAGTATTTGGTTAAAACAGTGCTAAAGCTATCATCATGGTCTAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGCTACTGGTTTTATCGCGCGTCGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATAGTTTATTACGTCGAATATGAAGCATAATGGGGCCATTATGCCACCACAGGCCACAACACTTCAGGATATGATA
ATGATCCACCGACACGCACTGCCCGGCCAGTACTTTCTTCGTGAGGGATTGCAGGGAAGGCAACAT
Mkuzi 1979 MGF 360 11L (AY261362.1:28164-29225)
SEQ ID No. 79
TTATCTTTGTTCATAATCAAGAAAAATCCCCATATTTATTGAGTGATAATTTTTTAACATGTAATTTATTTTTTCAGGGTCCA
TAACGATCGACAACAGAGAAATGACCGGATTGTAATGCTTTAATGATAAGGCATGGGCTATCAGATAATTTCCCTTTTGTTCT
GCCAAAGCTTTGCCCTCCTCAAAGGCGTTGGCGCCCAGGTCTATACAAAAGAACAGGTTTCCAAGATTATAGTTTTGTATGGA
AACCAGCATAGCTTGATTGATGTTGGCTCCCATGATAAAACAGTAGTAAATGACCGAATAGCTATAATCTTGGATGCAGGCTA
TGTGCATCATTTCATCAATATCCATGCGGACCCTTTCTATTTCATACAGCTCGTGAAGGTCGAACACGTTGTTGTAAAAAAGG
GCGCACATGAGCCGCCACCTATGTAGGCGCGGGTATTTCTGGTAAAAGTAGCGAATAGCATCTTTGAGGTCATAGTCCACCGC
TATAGCATACCAGTATTTGGTTAAAACAGTGCTAAAGCTATCATCATGGTCTAGCATGAAGGTTATCTCCATGAGCCCTCTTA
ACTCCCACATGATTTCTCCCCTCAGATCCAGATTATCTATAATCCTTAAATTGGGGTTATTGGAAAACACCTCGTGGCAAAAG
ATAATATTGCTACTGGTTTTATCGCGCGTCGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATAGTTTATTACGTCGAATATGAAGCATAATGGGGCCATTATGCCACCACAGGCCACAACACTTCAGGATATGATA
ATGATCCACCGACACGCACTGCCCGGCCAGTACTTTCTTCGTGAGGGATTGCAGGGAAGGCAACAT
Warmbaths MGF 360 11L (AY261365.1:26388-27449)
SEQ ID No. 80
TTATCTTTGTTCATAATCAAGAAAAATCCCCATATTTATTGAGTGATAATTTTTTAACATGCGATTTATTTTTTCAGGGTCCG
TAACCACCGACAACAGAGAAATGACCGGATTGTAATGCTTTAATGATAAGGCATTGGCTATCAGATAATTTCCCTTTTGTTCT
GCCAAAGCTTTGCCCTCCTCAAAGGCGTTGGCACCCAGGTCTATACAAAAGAACAGGTTCCCAAGATTATAGTTTTGTATGGA
AACCAGCATGGCTTGATTGATGTTGGCTCCCATGATAAAACAGTAGTAGATGGCTGAATAGCTATAATCTTGGACGCAGGCTA
TGTGCATCATTTCATCAATGTCCATGCGGACCCTTTCTATTTCATACAGCTCGTGAAGGTCGAACACGTTGTTGTAAAAAAGG
GCGCACATGAGCCGCCACCTATGTAGCCGCGGGTATTTCTGGTAAAAGTAGCGGATAGCATCTTTGAGGTCATAGTCCACCGC
TATCGCGTACCAGTATTTGGTTAAAACAGTGCTAAAGCTATCATCATGGTCCAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGTTACTGGTTTTATCGCGCGTTGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATATTCTATTACGTCGAATATGAAGCATTATGGGGCCATTATGCCACCACAGGCCACAACACTTCAGGATATGATA
ATGATCCACCGACACGCACTGCCCGGCCAGTACTTTCTTCGTGAGGGATTGCAGGGAAGGCAACAT
Pretorisuskop/96/4 MGF 360 11L (AY261363.1:27214-28275)
SEQ ID No. 81
TTATCTTTGTTCATAATCAAGAAAAATCCCCATATTTATTGAGTGATAATTTTTTAACATGCGATTTATTTTTTCAGGGTCCG
TAACCACCGACAACAGAGAAATGACCGGATTGTAATGCTTTAATGATAAGGCATTGGCTATCAGATAATTTCCCTTTTGTTCT
GCCAAAGCTTTGCCCTCTTCAAAGGCGTTGGCACCCAGGTCTATACAAAAGAACAGGTTCCCAAGATTATAGTTTTGTATGGA
AACCAGCATGGCTTGATTGATGTTGGCTCCCATGATAAAACAGTAGTAAATGGCTGAATAGCTATAATCTTGGACGCAGGCTA
TGTGCATCATTTCATCAATGTCCATGCGGACCCTTTCTATTTCATACAGCTCGTGAAGGTCGAACACGTTGTTGTAAAAAAGG
GCGCACATGAGCCGCCACCTATGTAGCCGCGGGTATTTCTGGTAAAAGTAGCGGATAGCATCTTTGAGGTCATAGTCCACCGC
TATCGCGTACCAGTATTTGGTTAAAACAGTGCTAAAGCTATCATCATGGTCCAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGTTACTGGTTTTATCGCGCGTTGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATATTCTATTACGTCGAATATGAAGCATTATGGGGCCATTATGCCACCACAGGCCACAACACTTCAGGATATGATA
ATGATCTACCGACACGCACTGCCCAGCCAGTACTTTCTTCGTGAGGGATTGCAGGGAAGGCAACAT
Tengani 62 MGF 360 11L (AY261364.1:21627-22688)
SEQ ID No. 82
TTATCTTTGTTCATAATCAAGAAAAATCCCCATATTTATTGAGTGATAATTTTTTAACATGCAATTTATTTTTTTAGGGTCTG
TAACGACCGACAACAGAGAAATGACCGGATTGTAATGCTTTAATGATAAGGCATCGGCTATCAGATAATTTCCCTTTTGTTCT
GCCAAAGCTTTGCCTTCCTCAAAGGCGTTGGCTCCCAGGTCTATACAAAAGAACAGGTTCCCAAGATTATAGTTTTGTATGGA
AACCAGCATGGCTTGATTGATGTTGGCTCCCATGATAAAACAGTAGTAAATGGCCGAATAGCTATAATCTTGGACGCAGGCTA
TGTGCATCATTTCATCAATGTCCATGCGGACCCTTTCTATTTCGTACAGCTCGTGAAGGTCGAACACGTTGTTGTAAAAAAGG
GCGCACATGAGCCGCCACCTATGTAGACGCGGGTATTTCTGGTAAAAGTAGCGGATCGCATCTTTGAGGTCATAGTCCACCGC
TATCGCGTACCAGTATTTGGTTAAAACAGTGCTAAAGCTATCATCATGGTCCAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGTTACTGGTTTTATCGCGCGTCGTATCAAAGAAAATTTTTAAAATATACTCTCTTTCTAAATATTCTTTGGCACC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATAGTTTATTACGTCGAATCTGAAGCATAATGGGTCCATTATGCCACCACAGGCCACAACACTTCAGGATATGATA
ATGATCCACCGACACGCACTGCCCGGCCAGTACTTTCTTCGTGAGGGATTGCAGGGAAGGCAACAT
Warthog MGF 360 11L (AY261366.1:23157-24218)
SEQ ID No. 83
TTATCTTTGTTCATAATCAAGAAAAATCCCCATATTTATTGAGTGATAATTTTTTAACATGCGATTTATTTTTTCAGGGTCCG
TAACCACCGACAACAGAGAAATGACCGGATTGTAATGCTTTAATGATAAGGCATTGGCTATCAGATAATTTCCCTTTTGTTCT
GCCAAAGCTTTGCCCTCCTCAAAGGCGTTGGCCCCCAGGTCTATACAAAAGAACAGGTTCCCAAGATTATAGTTTTGTATGGA
AACCAGCATGGCTTGATTGATGTTGGCTCCCATGATAAAACAGTAGTAAATGGCTGAATAGCTATAATCTTGGACGCAGGCTA
TGTGCATCATTTCATCAATGTCCATGCGGACCCTTTCTATTTCATACAGCTCGTGAAGGTCGAACACGTTGTTGTAAAAAAGA
GCGCACATGAGCCGCCACCTATGTAGCCGCGGGTATTTCTGGTAAAAGTAGCGGATAGCATCTTTGAGGTCATAGTCCACCGC
TATCGCGTACCAGTATTTGGTTAAAACAGTACTAAAGCTATCATCATGGTCCAGCATGAAGGTTATCTCCATGAGCCCTCTTA
ACTCCCACATGATTTCTCCCCTTAGATCCAGATTATCTATAATCCTTAAATTGGGGTTATTGGAAAACACCTCGTGGCAAAAG
ATAATATTGTTACTGGTTTTATCGCGCGTTGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATATTCTATTACGTCGAATATGAAGCATTATGGGGCCATTATGCCACCACAGGCCACAACACTTCAGGATATGATA
ATGATCCACCGATACGCACTGCCCGGCCAGTACTTTCTTCGTGAGGGATTGCAGGGAAGGCAACAT
Malawi Lil-20/1 (1983) MGF 360 11L (AY261361.1:24831-25892)
SEQ ID No. 84
TTATCTTCGTTCATAATCAAGAAAAACCCTCATATTTATTGAGTGATAATTTTTTAACATGCAATTTATTTTTTCAGGGTCCA
TAATGACCGACAACAGAGAAATGACCGGATTGTAATGCTTTAATGATAAGGCATCGGCTATCAGATAATTTTCCTTTTGTTCT
GCCAAAGCTTTCCCCTCCTCAAAGGCGTTGGCCCCCAGATCTATACAAAAGAACATGTTCCCAAGATTATAGTTTTGTATAGA
AACAAGCATGGCTTGATTGATGTTGGCCCCCATGATAAAACAGTAATAAATAGCCGCATAGCTATAATCTTGGACGCAGGCTA
TGTGCATCATTTCATCAATATCCATGCGGACCCTTTCTATTACATACAGCTCGTGAAGGTCGAACACGTTGTTGTAAAAAAGG
GCGCACATAAGCCGCCAGTGGTGTAGATGGGTATACCTCTGATAAAAATAATGGATAGCCTTTTTGAGGCCATAGTCTACCGC
TATGGCGTACCAGTATTTGGTTAACATATTGCTAAAGGAGTTGTCATGGTCCAACATGTAGGTTATCTCCATGAGTCCCCTTA
GCTCCCACATGATTTCCTCCCTCAGATCCAAATTATCCACATTTTTCAGGTTGGGGTTATTGGAAAACATTTCGTGGCATAAG
ATAATATTACTGTCGGTTTTGTTGCGCGTCGTATCAAAGAAAATTTTTAAAATATAATCTCTATCTAAACGATCTTTGGCTCC
TAGTTCTCTGCATAGATTACGGGTGTGCTCCGTATGGGCACAAATCAGTCCATAATTAATATCGGCACCCCACTCGGCAAACA
GCCTTATCAAGTCATGGTTGTTTTCCTTCGCGGCTTTCATCAGTACGGTGTTCAATTCAATACCCTCGCTAAAACAGGTTGAC
CTTATAAACATTTTGTGACGATAAACGTAAAATGTAATAGGGCCATCATACCACCACAACCCGCAGCACTTTAAGACATGATA
TTGATCTACCGGTACACACTGTTTGGCCAGCACTTTCTTAGTTAAAGATTGTAAGGAAGGCAACAT
Kenya 1950 MGF 360 11L (AY261360.1:30655-31716)
SEQ ID No. 85
TTATCTGCGTTCATAATCGAGAAAAACCACCATATTCTTTGAGTGATAATTTTTTAACATAGAATTTATCTTTTCTGGGTCGG
TCACGGCCGACAACAGGGAAATCACCGGGTTATAATGTTTTAATGATAAGGCATCGGCTATCAAATAATTTTCCTTTTGTATT
GCTAGGGTTTTGCCTTCTTCAAAGGCGTTGGCACCCAGGTCTATACAAAAGAACATGTTCCCGAGATTATAGTTTTGTATGGA
AACAAACATGGCTTGATTGATGTTGGCCCCCATGATAAAACAGTAGTAGATAGCTGAATAGCTATAATCTTGGATGCAGGCTA
TGTGCATCATCTCATCGATATCCATGCGGACCCTTTCTATTTCATACAGCTCGTGAAGGTCGAACACATTGTTATAAAAAAGG
GCGCACATAAGCCGCCACCGATGTAGATGGGTATACTTCTGATAAAAATAGTGGATGGCCTTTTTGAGGCCGTAGTCTACCGC
TATGGCGTACCAGTATTTGGTTAACATATTGCTGAAGGAGTTGTCATGGTCCAACATGTAGGTTATCTCCATGAGTCCCCTTA
GCTCCCACATGATTTCCTCCCTCAGATCCAAATCATCCACATGTTTCAGATTGGGGTTATTGGAAAACATTTCGTGGCATAAG
ATAATATTACTGTCGGTTTTGTTGCGCGTCGTATCAAAGAAAATTTTTAAAATATACTCTCTATCTAAACGGTCTTTGGCTCC
AAGTTCTCTGCATAGATTGCGGGTGTGCTCCGAATGAGCACAAACCAGTCCATAATTAATATCGGCACCCCACTCAACAAACA
GCCTTATCAAGTCATGATTGTTTTCCTTCGCGGCTTTCATCAGTACGGTGTTCAATTCAATACCCTCGCTAAAACAGGTTGAC
TTTATAAACATTTTGTTACGATACGTATAAAATGTAATAGGGCCATCATACCACCACAACCCGCAGCATTTCAGGATATGATA
TTGATCTACCGGTATACACTGTTTGGCCAGCACTTTCTTGGATAGGGATTGCAAGGAAGGCAACAT
Ken05/Tk1 MGF 360 11L (NC_044945.1:28407-29468)
SEQ ID No. 86
TTATCTGCGTTCATAATCGAGAAAAACCACCATATTCTTTGAGTGATAATTTTTTAACATAGAATTTATCTTTTCTGGGTCGG
TCACGGCCGACAACAGGGAAATCACCGGGTTATAATGTTTTAATGATAAGGCATCGGCTATCAAATAATTTTCCTTTTGTATT
GCTAGGGCTTTGCCTTCTTCAAAGGCGTTGGCACCCAGGTCTATACAAAAGAACATGTTCCCGAGATTATAGTTTTGTATGGA
AACAAACATGGCTTGATTGATGTTGGCCCCCATGATAAAACAGTAGTAAATAGCTGAATAGCTATAATCTTGGATGCAGGCTA
TGTGCATCATCTCATCGATATCCATGCGGACCCTTTCTATTTCATACAGCTCGTGAAGGTCGAACACATTGTTATAAAAAAGG
GCGCACATAAGCCGCCACCGATGTAGATGGGTATACTTCTGATAAAAATAGTGGATGGCCTTTTTGAGGCCGTAGTCTACCGC
TATGGCGTACCAGTATTTGGTTAACATATTGCTGAAGGAGTTGTCATGGTCCAACATGTAGGTTATTTCCATGAGTCCCCTTA
GCTCCCACATGATTTCCTCCCTCAGATCCAAATCATCCACATGTTTCAGATTGGCGTTATTGGAAAACATTTCGTGGCATAAG
ATAATATTACTGTCGGTTTTGTTGCGCGTCGTATCAAAGAAAATTTTTAAAATATACTCTCTATCTAAACGGTCTTTGGCTCC
AAGTTCTCTACATAGATTGCGGGTGTGCTCCGAATGAGCACAAACCAGTCCATAATTAATATCGGCACCCCACTCAACAAACA
GCCTTATCAAGTCATGATTGTTTTCCTTCGCGGCTTTCATCAGTACGGTGTTCAATTCAATACCCTCGCTAAAACAGGTTGAC
TTTATAAACATTTTGTTACGATACGTATAAAATGTAATAGGGCCATCATACCACCACAACCCGCAGCATTTCAGGATATGATA
TTGATCTACCGGTATACACTGTTTGGCCAGCACTTTCTTGGATAGGGATTGCAAGGAAGGCAACAT
Ken06.Bus MGF 360 11L (NC_044946.1:24534-25595)
SEQ ID No. 87
TTATCTGCGTTCATAATCGAGAAAAACCACCATATTCTTTGAGTGATAATTTTTTAACATAGAATTTATCTTTTCCGGGTCGG
TCACGGCCGACAACAGGGAAATCACCGGGTTATAATGTTTTAATGATAAGGCATCGGCTATCAAATAATTTTCCTTTTGTATT
GCTAGGGCTTTGCCTTCTTCAAAGGCGTTGGCACCCAGGTCTATACAAAAGAACATGTTCCCGAGATTATAGTTTTGTATGGA
AACAAACATGGCTTGATTGATGTTGGCACCCATGATAAAACAGTAGTAAATAGTTGAATAGCTATAATCTTGGATGCAGGCTA
TGTGCATCATCTCATCGATATCCATGCGGATCCTTTCTATTTCATACAGCTCGTGAAGGTCGAACACATTGTTATAAAAAAGG
GCGCACATGAGCCGCCACCGATGTAGATGGGTATACTTCTGATAAAAATAGTGGATGGCCTTTTTGAGGCCGTAGTCTACCGC
TATGGCGTACCAGTATTTGGTTAACATGTTGCTGAAGGAGTTGTCATGGTCCAACATGTAGGTTATCTCCATGAGTCCCCTTA
GCTCCCACATGATTTCCTCCCTCAGGTCCAAATCATCCACATGTTTCAGATTGGGGTTATTGGAAAACATTTCGTGGCATAAG
ATAATATTACTGTCGGTTTTGTTGCGCGTCGTATCAAAGAAAATTTTTAAAATATACTCTTTATCTAAACGGTCTTTGGCCCC
TAGTTCTCTGCATAGATCGCGGGTGTGCTCCGAATGAGCACAAACCAGTCCATAATTAATATCGGCACCCCACTCAGTAAATA
ATTTTATTAGGTCATGATTGTTTTCCTTCGCGGCTTTCATCAGTACGGTGTTCAATTCAATACCCTCGCTAAAACAGGTTGAC
TTTATAAACATTTTGTTACGATACGTATAAAATGTAATAGGGCCATCATACCACCACAACCCGCAACTTTTCAGGATATGATA
TTGATCTACCGGTATACACTGTTTGGCCAGCACTTTTTTGGATAGGGATTGCAAGGAAGGCAACAT

In an embodiment the attenuated ASFV of the invention comprises a functional version of MGF 360 11L. Suitably the functional version of MGF 360 11L comprises the sequence of SEQ ID No. 75, 76, 77, 78, 79, 80, 81, 82, 83, 84, 85, 86 or 87. Suitably the functional version of MGF 360 11L comprises a sequence having at least 70%, at least 80%, at least 35 90% or at least 95% identity with SEQ ID No. 75, 76, 77, 78, 79, 80, 81, 82, 83, 84, 85, 86 or 87. Suitably the functional version of MGF 360 11L consists of the sequence of SEQ ID No. 75, 76, 77, 78, 79, 80, 81, 82, 83, 84, 85, 86 or 87.

Georgia 2007/1 MGF 360 12L (LR743116.1:30355-31407)
SEQ ID No. 88
TCATCTTAAATCATAGGTAAGGAAGATCATCATATTTTTTGAAACGTAATTTTTTAACGCATGATCTATGATTTCAGGGTCCG
TGCTTTTAGGCAACGGGGTGGTGGCCGGACTATAAATCTTTAGGGATAAAATGTTCTTTATAAGCTCATACCCTTCCCCTAAA
GCTGTAGTACCCTCTTCGAAAACATCAGCCCCCAGATCTATACAAAAGAACATGTTTTCTATATTATAGTACTGTATTGAGCT
AAGCATGGCTTGATTGATGTTGGCGCCCAGGACATAGCAGTAGTACATGGTTGAAAGGTTGTGGTCTTTGATGCAGGCGATCC
GCATCATCTCTTCTATGTCCATATGGATCTTGTCCTTTTCATACGCCTCATGAAGGTCAAACACATTATTAAAACAAAGAGCA
CATGTTAACCGCCACGTATTCAGGTGTGTATATTTTTGGTAAAAATACTGTATGGCCTCTTTCAGGTTATAGCGTATGGCTAT
AGCGTACCAGTATTTGAGTAGTAATGTACTGAGCGAAAACTCATTATTTAGCAGATCGGTTTTTACTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTTTCCTTGGCACCTAG
CTCTCGACAGAGGTCCCAGGTGTGCTCCGTGTTGACAGATACCAGCCCGTAGTTGATGTCCGCCCCCCACTCTGCAAACAGTT
TTATAAGGTTGTAGTTGTTTTCCCTTACAGCCTTCACTAACGCCGTATTTAGGTTTAAGCCCTCTTTAATACCTGCTGATTTT
ATGAGCCTTAGGTTATGATCAAACGTGATCGGAGCATCATGCCACCATAGGTCATAACACTTTAAAAGATAATGTTGGTTCGT
GGGCACGCATTGTCCAGCCAACACCTTTTTGGTCAGAGATTGCAGGGAAGGCAACAT
China/2018/AnhuiXCGQ MGF 360 12L (MK128995.1:29384-30436)
SEQ ID No. 89
TCATCTTAAATCATAGGTAAGGAAGATCATCATATTTTTTGAAACGTAATTTTTTAACGCATGATCTATGATTTCAGGGTCCG
TGCTTTTAGGCAACGGGGTGGTGGCCGGACTATAAATCTTTAGGGATAAAATGTTCTTTATAAGCTCATACCCTTCCCCTAAA
GCTGTAGTACCCTCTTCGAAAACATCAGCCCCCAGATCTATACAAAAGAACATGTTTTCTATATTATAGTACTGTATTGAGCT
AAGCATGGCTTGATTGATGTTGGCGCCCAGGACATAGCAGTAGTACATGGTTGAAAGGTTGTGGTCTTTGATGCAGGCGATCC
GCATCATCTCTTCTATGTCCATATGGATCTTGTCCTTTTCATACGCCTCATGAAGGTCAAACACATTATTAAAACAAAGAGCA
CATGTTAACCGCCACGTATTCAGGTGTGTATATTTTTGGTAAAAATACTGTATGGCCTCTTTCAGGTTATAGCGTATGGCTAT
AGCGTACCAGTATTTGAGTAGTAATGTACTGAGCGAAAACTCATTATTTAGCAGATCGGTTTTTACTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTTTCCTTGGCACCTAG
CTCTCGACAGAGGTCCCAGGTGTGCTCCGTGTTGACAGATACCAGCCCGTAGTTGATGTCCGCCCCCCACTCTGCAAACAGTT
TTATAAGGTTGTAGTTGTTTTCCCTTACAGCCTTCACTAACGCCGTATTTAGGTTTAAGCCCTCTTTAATACCTGCTGATTTT
ATGAGCCTTAGGTTATGATCAAACGTGATCGGAGCATCATGCCACCATAGGTCATAACACTTTAAAAGATAATGTTGGTTCGT
GGGCACGCATTGTCCAGCCAACACCTTTTTGGTCAGAGATTGCAGGGAAGGCAACAT
Tengani 62 MGF 360 12L (AY261364.1:24540-25592)
SEQ ID No. 90
TCATCTTAAATCATAGGTAAGGAAGATCATCATATTTTTTGAAACGTAATTTTTTAACGCATGATCTATGATTTCAGGGTCCA
TGCTTTTAGGCAACGGGGTGGTGGCCGGACTATAAATCTTTAGGGATAAAATGTTCTTTATAAGCTCATACCCTTCCCCTAAA
GCTGTAGTACCCTCTTCGAAAACATCAGCCCCCAGATCTATACAAAAGAACATGTTTTCTATATTATAGTACTGTATTGAGCT
AAGCATGGCCTGATTGATGTTGGCGCCCAGGACATAGCAGTAGTACATGGTTGAAAGGTTGTGGTCTTTGATGCAGGCGATCC
GCATCATCTCTTCTATGTCCATATGGATCTTGTCCTTTTCATACGCCTCATGAAGGTCAAACACATTATTAAAACAAAGAGCA
CATGTTAACCGCCACGTATTCAGGTGTGTATATTTTTGGTAAAAATACTGTATGGCCTCTTTCAGGTTATAGCGTATGGCTAT
AGCGTACCAGTATTTGAGTAGTAATGTACTGAGCGAAAACTCATTATTTAGCAGATCGGTTTTTTCTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTCTCCTTGGCACCTAG
CTCTCGACAGAGGTCCCAGGTGTGCTCCGTGTTGACAGATACCAGCCCGTAGTTGATGTCCGCCCCCCACTCTGCAAACAGTT
TTATAAGGTTGTAGTTGTTTTCCCTTACAGCCTTTACTAACGCCGTATTTAGGTTTAAGCCCTCTTTAATACCTGCTGATTTT
ATGAGCCTTAGGTTATGATCAAACGTGATCGGAGCATCATGCCACCATAGGTCATAACACTTTAAAAGATAATGTTGGTTCGT
GGGCACGCATTGTCCAGCCAACACCTTTTTGGTCAGAGATTGCAGGGAAGGCAACAT
Warthog MGF 360 12L (AY261366.1:26036-27088)
SEQ ID No. 91
TCATCTTAAATCGTAGGTAAGGAAGATCATCATATTTTTTGAAACGTAATTTTTTAACGCATGATCTATAATTTCAGGGTCCG
TGCTTTTAGGCAACGGGGTGGTGGCCGGACTATAAATCTTTAGGGATAAAATGTTCTTTATAAGCTTATACCCTTCCCCTAAA
GCTATAGTGCCCTCTTCGAAAGCATCAGCCCCCAGATCCATACAAAAGAACATGTTTTCTATATTATAGTACTGTATTGAGGT
AAGCATGGCCTGATTGATGTTGGCGCCCAGGACATAGCAGTAGTACATGGTTGAAAGGTTGTGGTCTTTGATGCAGGCGATCC
GCATCATCTCTTCTATGTCCATATGGATCTTGTCCTTTTCATACGCCTCATGAAGGTCAAACACATTATTAAAACAAAGAGCA
CATGTTAACCGCCACGTATTCAGGTGTGTATATTTTTGGTAAAAATACTGTATGGCCTCTTTCAGGTTATAGCGTATGGCTAT
AGCGTACCAGTATTTGAGTAGTAATGCACTGAGCGAAAACTCATTATTTAGCAGATCGGTTTTTTCTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTCTCCTTGGCACCTAG
CTCTCGACAGAGGTCCCGGGTGTGCTCCGTGTTGACAGATACCAGCCCATAGTTGATGTTCGCCCCCCACTCTGCAAACAGTT
TTATAAGGTTGTAATTGTTTTCCCTTACAGCCTTCACTAACGCCGTATTTAGGTCTAAGCCCTCTTTAATGCCTGCTGATTTT
ATGAGCTTTAGGTTATGATCAAACGTGATCGGACCATCATGCCACCATAGGTCATAACACTTTAAAAGATAATGTTGGTTCGT
GGGCACGCATTGTCCAGCCAACACCTTTTTGGTCAGAGATTGCAGGGAAGGCAACAT
Warmbaths MGF 360 12L (AY261365.1:29294-30346)
SEQ ID No. 92
TCATCTTAAATCGTAGGTAAGGAAGATCATCATATTTTTTGAAACGTAATTTTTTACCGCATGATCTATGATTTCAGGGTCCG
TGCTTTTAGGCAACGGGGTGGTGGCCGGACTATAAATCTTTAGGGATAAAATGTTCTTTATAAGCTTATACCCTTCCCCTAAA
GCTATAGTACCCTCTTCGAAAGCATCAGCCCCCAGATCCATACAAAAGAACATGTTTTCTACATTATAGTACTGTATTGAGGT
AAGCATGGCCTGATTGATGTTGGCGCCCAGAACATAGCAGTAGTACATGGTTGAAAGGTTGTGGTCTTTGATGCAGGCGATCC
GCATCATCTCTTCTATGTCCATATAGATCTTGTCCTTTTCATACGCCTCATGAAGGTCAAACACATTATTAAAACAAAGAGCA
CATGTTAACCGCCATGTATTCAGGTGTGTATATTTTTGGTAAAAATACTGTATGGCCTCTTTCAGGTTATAGCGTATGGCTAT
AGCGTACCAGTATTTGAGTAGTAATGTACTGAGCGAAAACTCATTATTTAGCAGATCGGTTTTTTCTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTCTCCTTGGCACCTAG
CTCTCGACAGAGGTCCCGGGTGTGCTCCGTGTTGACAGATACCAGCCCATAGTTGATGTCCGCCCCCCACTCTGCAAACAGTT
TTATAAGGTTGTAGTTGTTTTCCCTTACAGCCTTCACTAACGCCGTATTTAGGTCTAAGCCCTCTTTAATGCCTGCTGATTTT
ATGAGCTTTAGGTTATGATCAAACGTGATCGGACCATCATGCCACCATAGGTCATAACACTTTAAAAGATAATGTTGGTTCGT
GGGCACGCATTGTCCAGCCAACACCTTTTTGGTCAGAGATTGCAGGGAAGGCAACAT
Pretorisuskop/96/4 MGF 360 12L (AY261363.1:30098-31150)
SEQ ID No. 93
TCATCTTAAATCGTAGGTAAGGAAGATCATCATATTTTTTGAAACGTAATTTTTTAACGCATGATCTATAATTTCAGGGTCCG
TGCTTTTAGGCAACGGGGTGGTGGCCGGACTATAAATCTTTAGGGATAAAATGTTCTTTATAAGCTTATACCCTTCCCCTAAA
GCTATAGTGCCCTCTTCGAAAGCATCAGCCCCCAGATCCATACAAAAGAACATGTTTTCTATATTATAGTACTGTATTGAGGT
AAGCATGGCCTGATTGATGTTGCCCCCCAGGACATAGCAGTAGTACATGGTTGAAAGGTTGTGGTCTTTGATGCAGGCGATCC
GCATCATCTCTTCTATGTCCATATGAATCTTGTCCTTTTCATACGCCTCATGAAGGTCAAACACATTATTAAAACAAAGAGCA
CATGTTAACCGCCACGTATTCAGGTGTGTATATTTTTGGTAAAAATACTGTATGGCCTCTTTCAGGTTATAGCGTATGGCTAT
AGCGTACCAGTATTTAAGTAGTAATGTACTGAGCGAAAACTCATTATTTAGCAGATCGGTTTTTTCTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTCTCCTTGGCACCTAG
CTCTCGACAGAGGTCCCAGGTGTGCTCCGTGTTGACAGATACCAGCCCATAGTTGATGTTCGCCCCCCACTCTGCAAACAGTT
TTATAAGGTTGTAGTTGTTTTCCCTTACAGCCTTCACTAACGCCGTATTTAGGTCTAAGCCCTCTTTAATGCCTGCTGATTTT
ATGAGCTTTAGGTTATGATCAAACGTGATCGGACCATCATGCCACCATAGGTCGTAACACTTTAAAAGATAATGTTGGTTTGT
GGGCACGCATTGTCCAGCCAACACCTTTTTGGTCAGAGATTGCAGGGAAGGCAACAT
Mkuzi 1979 MGF 360 12L (AY261362.1:31073-32125)
SEQ ID No. 94
TCATCTTAAATCATAGGAAAGGAAGATCATCATATTTTTTGAAAAGTAATTTTTTAACGCATGATCTATGATTTCAGGGTCCG
TGCTTTTAGGCAACGGGATGGTGGTCGGACTATAAATCTTTAGAGATAAAATGTTTTTTATAAGCTCATACCCTTCCCCTAAA
GCTGTAGTACCCTCTTCGAAAACATCAGCCCCCAGATCCATACAAAAGAACATGTTTTCTATATTATAGTACTGTATTGAGGT
AAGCATGGCCTGATTGATGTTGGCGCCCAACATATAGCAGTAGTACATGGTTGAAAGGTTGTGGTCTTTAATGCAGGCGATCC
GCATCATCTCTTCTATGTCCATATAGATCTTGTCCTTTTCATACGCCTCATGGAGGTCAAACACATTATTAAAACAAAGAGCA
CATGTTAACCGCCACGTATTCAGGTGTGTATATTTTTGGTAAAAATACTGTATGGCTTCTTTCAGGTTATAGCGTATGGCTAT
AGCGTACCAGTATTTGAGTAGCAATGTACTGAGCGAAAACTCATTATTTAGCAGATCGGTTTTTTCTATTAACTCCCTTAACT
CCCAAAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTTTTCATTCAAGGTCTCCTTGGCACCTAG
CTCTCGACAGAGGTCCCAGGTGTGCTCCGAGCTGACAGATACCAGCCCATAGTTGATGTCCGCCCCCCACTCTGCAAACAATT
TTATAAGGTTGTAGTTGTTTTCCTTTACAGCCTTCACCAACGCCGTATTTAGGTCTAAGCCCTCTTTAATGCCTGCTGATTTT
ATGAGCCTTAGGTTATGATCAAACGTGATCGGAGCATCATGCCACCATAGGTCATAATATTTTAAAAGATAATGTTGGTTCGT
GGGCACGCATTGTCCGGCCAACACCTTTTTGGTCAAAGATTGCAGGGAAGGCAACAT
L60 MGF 360 12L (NC_044941.1:23965-25017)
SEQ ID No. 95
TCATCTTAAATCATAGGAAAGGAAGATCATCATATTTTTTGAAAAGTAATTTTTTAACGCATGATCTATGATTTCAGGGTCCG
TGCTTTTAGGCAACGGGATGGTGGTCGGACTATAAATCTTTAGAGATAAAATGTTTTTTATAAGCTCATACCCTTCCCCTAAA
GCTGTAGTACCCTCTTCGAAAACATCAGCCCCCAGATCCATACAAAAGAACATGTTTTCTATATTATAGTACTGTATTGAGGT
AAGCATGGCCTGATTGATGTTGGCGCCCAACATATAGCAGTAGTACATGGTTGAAAGGTTGTGGTCTTTGATGCAGGCAATCC
GCATCATCTCTTCTATGTCCATATGGATCTTGTCCTTTTCATACGCCTCATGGAGGTCAAACACATTATTAAAACAAAGAGCA
CATGTTAACCGCCACGTATTCAGGTGTGTATATTTTTGGTAAAAATACTGTATGGCCTCTTTCAGGCTATAGCGTACGGCTAT
AGCGTACCAGTATTTGAGTAGTAATGTACTGAGCAAAAACTCATTATTTAGCAGATCGGTTTTTTCTATTAACTCCCTTAACT
CCCAGAAAATTTCTATCCTCAATTTCAGATTATTTACTTTTTGTAATATCGGATTGTTGGAAAACACCTCATGGCATAAAATA
ATGTTACTACTAGTTTTATGAAACTTTAGATCTATGAAAATTTGTAAAATTTCCTCTTCATTCAAGATCTCCTTGGCACCTAA
CTCTTGACAGAGGTCCCGGGTATGCTCCGTGTTGACAGATACCAGCCCATAGTTGATGTCCGCCCCCCACTCTGTAAATAGTT
TTATAAGGCTGTAGTTGTTTTCCCTTACAGCCTTTACTAACGCCGTATTTAGGTCTAAGCCCTCTTTAATGCCTGATGATTTT
ATGAGCCTTAGGTTATGATCAAACGTGATCGGAGCATTATACCACCATAGATCATAATATTTTAAAAGATAATGTTGGTTCGT
GGGCACGCATTGTCCAGCCAACACCTTTTTGGTCAAAGATTGCAGGGAAGGCAACAT
Benin 97/1 MGF 360 12L (NC_044956.1:23616-24668)
SEQ ID No. 96
TCATCTTAAATCATAGGAAAGGAAGATCATCATATTTTTTGAAAAGTAATTTTTTAACGCATGATCTATGATTTCAGGGTCCG
TGCTTTTAGGCAACGGGATGGTGGTCGGACTATAAATCTTTAGAGATAAAATGTTTTTTATAAGCTCATACCCTTCCCCTAAA
GCTGTAGTACCCTCTTCGAAAACATCAGCCCCCAGATCCATACAAAAGAACATGTTTTCTATATTATAGTACTGTATTGAGGT
AAGCATGGCCTGATTGATGTTGGCGCCCAACATATAGCAGTAGTACATGGTTGAAAGGTTGTGGTCTTTGATGCAGGCAATCC
GCATCATCTCTTCTATGTCCATATGGATCTTGTCCTTTTCATACGCCTCATGGAGGTCAAACACATTATTAAAACAAAGAGCA
CATGTTAACCGCCACGTATTCATGTGTGTATATTTTTGGTAAAAATACTGTATGGCCTCTTTCAGGCTATAGCGTACGGCTAT
AGCGTACCAGTATTTGAGTAGTAATGTACTGAGCAAAAACTCATTATTTAGCAGATCGGTTTTTTCTATTAACTCCCTTAACT
CCCAGAAAATTTCTATCCTCAATTTCAGATTATTTACTTTTTGTAATATCGGATTGTTGGAAAACACCTCATGGCATAAAATA
ATGTTACTACTAGTTTTATGAAACTTTAGATCTATGAAAATTTGTAAAATTTCCTCTTCATTCAAGATCTCCTTGGCACCTAA
CTCTTGACAGAGGTCCCGGGTATGCTCCGTGTTGACAGATACCAGCCCATAGTTGATGTCCGCCCCCCACTCTGTAAATAGTT
TTATAAGGCTGTAGTTGTTTTCCCTTACAGCCTTTACTAACGCCGTATTTAGGTCTAAGCCCTCTTTAATGCCTGATGATTTT
ATGAGCCTTAGGTTATGATCAAACGTGATCGGAGCATTATACCACCATAGATCATAATATTTTAAAAGATAATGTTGGTTCGT
GGGCACGCATTGTCCAGCCAACACCTTTTTGGTCAAAGATTGCAGGGAAGGCAACAT
Malawi Lil-20/1 (1983) MGF 360 12L (AY261361.1:27682-28731)
SEQ ID No. 97
TCATCTTAAATCGTAGGAAAGGAAGGTCATCATATTTTTTGATTTATAATTTTTTAACACATGATCTATTATTTCAGGGTCCG
TGTTTTTAGGTAACGGGGCAGTGGACGGACTATAGATCTTTAGGGATAAAATGTTCCTTATAAGCTCATACCCTTCCCCTACA
GGCATATTCTCTTCGAAAACATCAGCCCCCAGATCCATACAAAAGAACATGTTTTCTATATTATAGTACTGTATTGAGGCAAG
CATGGCCTGATTAATGTTGGCGCCCAGGACATAGCAGTAGTATATCGTTGAAAGGTTGTGGTCTTTGATGCAGGCTACTCGCA
TCATCTCTTCTAAGTCCATATAGATCTTGTCCTTTTCATACGCCTCATGAAGGTCAAACACATTATTAAAACAAAGAGCACAT
GTTAACCGCCACGTATTCAGGTGTGTATATTTTTGATAAAAATACTGTATGGCCTCTTTCAGGTTATAGCGTACGGCTATAGC
GTACCAGTATTTGAGTAATAATGTATTGAGTGAAAACTCATTATTTAACAGATCGGTTTTTTTTATTAACTCCCTTAACTCCC
AGAAAATTTCTATCCTCATTTTCAGATTATTTACTTTTTGTAATAACGGATTGTTGGAAAACACCTCATGGCATAAAATAATG
TTACTACTAGTTTTATAAAACTTTAGATCGATGAAAATTCGTAAAATTTCCTCTTCATTTAAGGTCTCCTTGGCGCCTAGCTC
TCTGCAGAGGTCCCGGGTGTGCTCCGTGTTGACAGATACCAGCCCGTAGTTGATGTCCGCCCCCCACTCTGTAAAAAGCTTTA
TAAGGTTGTAGTTGTTTTCCCTTACAGCCTTTACTAACGCCGTATTTAGGTCTAAGCCATCTTTAATGCCTGCTGATTTTATC
AGCCCTAGGTTATGATCAAACGTGATCGGAGAATCATACCACCATAGGTCGTAACACTTTAAAAGATAATATTGGTCCGTCGG
CAGGCATTGTCCAGCCAGTACCTTTTTGGTCAGAGATTGCAGGGAAGGCAACAT
Kenya 1950 MGF 360 12L (AY261360.1:33549-34604)
SEQ ID No. 98
TCATCTTAGATCATAAGTAAGGAAGACCATCATATTTTTTGAAAAGTAATTTTTTAACTCATGATCTATAATTTCAGGGTCCG
TGCTTTTAGGCAACGGGGTGGTGGCCGGGCTATAAATCTTTAGGGATAAAATATTCCTTATAGGCTCATACCCTTCCTGTCCC
ACTAGAGCCATACCCTCTTCAAAAGCATCGGCCCCCAGATCCATACAAAAGAATATGTTTTCTATATTATAGTACTGTATTGA
GGTAAGCATGGCCTGATTGATGTTGGCGCCCAGCATATAGCAGTAGTATATCGTTGAAAGGCTGTGGTCTTTGATGCAGGCTA
TCCGCATCATCTCTTCTAAATCCATACAGATCTTGTCCTTTTCATACGCCTCATGAAGGTCAAACACGTTATTAAAACAAAGA
GCACATGTTAATCGCCACGTATTCAGGTGCGTATATTTTTGGTAAAAATACTGTATGGCCTCTTTCAAGTTGTAGCGTACGGC
TATAGCGTACCAGTATTTGAGTAATAGTGTATTGAGCGAAAACTCATTATTTAGCAGATCGGTTTTTTCTATTATCTCCCTTA
ACTCCCAAAAAATTTCTATCCTCATTTTCAGGTTATTTACCTTTTTTAAAAGTAGATTATCGGAAAACACCTCATGACATAAG
ATAATGTTACTACTGGTTTTATGAAACTTTAAATCTATGAAAATTTGTAAAATATCCCCTTCATTTAAGGTCTTCTTGGCGCC
TAGCTCTCGACAGAGGTCCCAGGTGTGCTCCGTGTTGACAGATATCAGCCCGTAGTTGATGTTCGCTCCCCACTCCGTAAATA
GTTTTATAAGACTGTAGTTGTTTTCCTTTACAGCCTTCACTAATGCCATGTTTAGGTCTAAGCCTTCTTGAAGGCCTGCTGAT
TTTATCAGCCTTAGGTTATGATCAAACGTGATCGGAGCATTATTCCACCATAGATCGTAACACTTTAAAAGATAATGTTGGTC
CTCGGGCAGACATTGTCCAGCCAGCACCTTTTTGGTCAGAGATTGCAGGGAAGGCAACAT
Ken06.Bus MGF 360 12L (NC_044946.1:27428-28483)
SEQ ID No. 99
TCATCTTAGATCATAAGTAAGGAAGACCATCATATTTTTTGAAAAGTAATTTTTTAACGCATGATCTATAATTTCAGGGTCCG
TGCTTTTAGGCAATGGGGTAGTGGCCGGGCTATAAATCTTTAGGGATAAAATATTCCTTATAGGCTCATACCCTTCCTGTCCC
ACTAGAGCCATACCCTCTTCAAAAGCATCGGCCCCCAGATCCATACAAAAGAATATGTTTTCTATATTATAGTACTGTATTGA
GGTAAGCATGGCCTGATTGATGTTGGCGCCCAGCATATAGCAGTAGTATATTGTTGAAAGGTTGTGGTCTTTGATGCAGGCTA
TCCGCATCATCTCTTCTAAATCCATACAGATCTTGTCCTTTTCATACGCCTCATGAAGGTCAAACACGTTATTAAAACAAAGA
GCACATGTTAACCGCCACGTATTCAGGTGTGTATATTTTTGGTAAAAATACTGTATGGCCTCTTTCAAGTTGTAGCGTACGGC
TATAGCGTACCAGTATTTGAGTAATAGTGTATTGAGCGAAAACTCATTATTTAGCAGATCGGTTTTTTCTATTATCTCCCTTA
ACTCCCAAAAAATTTCTATCCTCATTTTCAGGTTATTTACCCTTTTTAAAAGTAAATTATCGGAAAACACCTCATGACATAAG
ATAATGTTACTACTGGTTTTATAAAATTTTAAATCTATGAAAATTTGTAAAATATCCCCTTCATTTAAGGTCTTCTTGGCGCC
TAGCTCTCGACAGAGGTCCCAGGTGTGCTCCGTGTTGACAGATATCAGCCCGTAGTTAATGTTCGCCCCCCACTCCGTAAATA
GTTTTATAAGACTGTAGTTGTTTTCCTTTACAGCCTTCACTAATGCCATGTTTAGGTCTAAGCCTTCTTGAATGCCTGCTAAT
TTTATCAGCCTTAGGTTATGATCAAACGTGATCGGAGCATTATTCCACCATAGATCGTAACACTTTAAAAGATAATGTTGGTC
CTCGGGCAGACATTGTCTAGCCAGCACCTTTTTGGTCAAAGATTGCAGGGAAGGCAACAT

In an embodiment the attenuated ASFV of the invention comprises a functional version of MGF 360 12L. Suitably the functional version of MGF 360 12L comprises the sequence of 50 SEQ ID No. 88, 89, 90, 91, 92, 93, 94 95, 96, 97, 98 or 99. Suitably the functional version of MGF 360 12L comprises a sequence having at least 70%, at least 80%, at least 90% or at least 95% identity with SEQ ID No. 88, 89, 90, 91, 92, 93, 94 95, 96, 97, 98 or 99. Suitably the functional version of MGF 360 12L consists of the sequence of SEQ ID No. 88, 89, 90, 91, 92, 93, 94 95, 96, 97, 98 or 99.

Georgia 2007/1 MGF 360 13L (LR743116.1:31568-32629)
SEQ ID No. 100
CTATAGTATATTATGAGAATATTCCCAAATGATGGATAAGTTTTTTGATTTATAATCTTTTAATAAACTGCTTATTTCTTCGG
GGTCCTTTAAGTTTAGTGGCAAGGAAGCATCTGAGCTGTAAATATCCAAAGCCAAACTATGGCTCAGAAAATTATAACCTTTT
TGTTCCGCTATGGCACGACCCTCTTCAAAGGCATTACCACCCAAATCTATACAGAAAAATATATTACCGATGTTATAATATTG
TACTGAAGTAAGCATAGCTTGGTTGATGTTGCCCCCCAGCGCGTAACAGTAATATATTGTTAATGGATTGTTATCCTTGGTAG
AAGCCAGACATATCATGTCATGGACGTCTATTTGGATGTTTTCCTTGTGGTACATCTCATGAAGCTCATATATTTTGTTATAA
TACAGGAGACATTTTAATCGCCATTCATTAAGATCCGTATATTTCTCATCTAGAAAACAAATGGCGTCCTTACAATCGTATTG
TACTGCTTTGGCGTACCAATACTTCACTAGTAAACCATTTAACTCGTCCGTTTCTTTTATTTCTATGAGCCCCCATAGTCTTT
TATAAATTAAGCCCCTTAATTGTATAACAAATTTGTTTTCTAAAATAGGATTATTCATAAAAATTTCATGGCACAAAATAATA
CTGCCGCTGGTTTTATTGTGCATTATCCTGGTAAAAATACGGAAAATATCGTTGTCCTCTAGAGTTTCTTTGGCGCCTAGCTG
TCTACACAACTCTCGGATGTGCTTCGTATTGATAGAAAGCAAACCATAGTTGATATTTGCGCCCCACTCTGTAAAGAGCTTTA
TCAGACTATAGTTGTTTTCCTTAACAGCTATTATTAATGCCACACGAAGGTCTATATCTTCTCCTAAAAATCCTGATTTTATT
TGTATTCGGCCACGATCCATACAAAGCTTGAGAGGAGCATCATGCCACCATAGGCCACAATATTTCAAAATGCAGTGTTCATC
TATTGACAAACACTGGCTGGCTATCGTCTTTTTGACGAGGGTCTGCAGAGAGAGCGGCAACGACAT
China/2018/AnhuiXCGQ MGF 360 13L (MK128995.1:30597-31658)
SEQ ID No. 101
CTATAGTATATTATGAGAATATTCCCAAATGATGGATAAGTTTTTTGATTTATAATCTTTTAATAAACTGCTTATTTCTTCGG
GGTCCTTTAAGTTTAGTGGCAAGGAAGCATCTGAGCTGTAAATATCCAAAGCCAAACTATGGCTCAGAAAATTATAACCTTTT
TGTTCCGCTATGGCACGACCCTCTTCAAAGGCATTACCACCCAAATCTATACAGAAAAATATATTACCGATGTTATAATATTG
TACTGAAGTAAGCATAGCTTGGTTGATGTTGCCCCCCAGCGCGTAACAGTAATATATTGTTAATGGATTGTTATCCTTGGTAG
AAGCCAGACATATCATGTCATGGACGTCTATTTGGATGTTTTCCTTGTGGTACATCTCATGAAGCTCATATATTTTGTTATAA
TACAGGAGACATTTTAATCGCCATTCATTAAGATCCGTATATTTCTCATCTAGAAAACAAATGGCGTCCTTACAATCGTATTG
TACTGCTTTGGCGTACCAATACTTCACTAGTAAACCATTTAACTCGTCCGTTTCTTTTATTTCTATGAGCCCCCATAGTCTTT
TATAAATTAAGCCCCTTAATTGTATAACAAATTTGTTTTCTAAAATAGGATTATTCATAAAAATTTCATGGCACAAAATAATA
CTGCCGCTGGTTTTATTGTGCATTATCCTGGTAAAAATACGGAAAATATCGTTGTCCTCTAGAGTTTCTTTGGCGCCTAGCTG
TCTACACAACTCTCGGATGTGCTTCGTATTGATAGAAAGCAAACCATAGTTGATATTTGCGCCCCACTCTGTAAAGAGCTTTA
TCAGACTATAGTTGTTTTCCTTAACAGCTATTATTAATGCCACACGAAGGTCTATATCTTCTCCTAAAAATCCTGATTTTATT
TGTATTCGGCCACGATCCATACAAAGCTTGAGAGGAGCATCATGCCACCATAGGCCACAATATTTCAAAATGCAGTGTTCATC
TATTGACAAACACTGGCTGGCTATCGTCTTTTTGACGAGGGTCTGCAGAGAGAGCGGCAACGACAT
Tengani 62 MGF 360 13L (AY261364.1:25753-26814)
SEQ ID No. 102
CTATAGTATATTATGAGAATATTCCCAAATGATGGATAAGTTTTTTGATTTATAATCTTTTAATAAACTGCTTATTTCTTCGG
GGTCCTTTAAGTTTAGTGGCAAGGAAGCATCTGAGCTGTAAATATCCAAAGCCAAACTATGGCTCAGAAAATTATAACCTTTT
TGTTCCGCTATGGCACGACCCTCTTCAAAGGCATTACCACCCAAATCTATACAGAAAAATATATTACCGATGTTATAATATTG
TACTGAAGTAAGCATAGCTTGGTTGATGTTGCCCCCCAGCGCGTAACAGTAATATATTGTTAATGGGTTGTTATCCTTGGTAG
AAGCCAGACATATCATGTCATGGACGTCTATTTGGATGTTTTCCTTGTGGTACATCTCATGAAGCTCATATATTTTGTTATAA
TACAGGAGACATTTTAATCGCCATTCATTAAGATCCGTATATTTCTCATCTAGAAAGCAAATGGCGTCCTTACACTCGTATTG
TACTGCTTTGGCGTACCAATACTTCACTAGTAAACCGTTTAACTCATCCGTTTCTTTTATTTCTATGAGCCCCCATAGTCTTT
TATAAATTAAGCCCCTTAATTGTATAGTAAATTTATCTTCTATATTAGGATTATTCATAAATATATCATGGCACAAAATAATA
CTGCCGCTGGTTTTATTGTGCATTATCTTGGTGAAAATACGGAAAATATCGTCGTCTTCTAGAGTTTCTTTGGCGCCTAGCTG
TCTACACAACTCTCGGATGTGCTTCGTATTGATAGAAAGCAAACTATAGTTGATATTTGCGCCCCACTCTGTAAAGAGCTTTA
TCAGACTATAGTTGTTTTCCTTAACAGCTATTATTAATGCTATATGGAGGTCTATATCTTCTCCTAAAAATCCTGATTTTATT
TGTATTTGGCCACGATCCATACAAAGCTTGAGGGGGACATCATGCCACCATAGGCCACAGTGTTTCAGAATGCAGTGTTCATC
TATCGACAAAGACTGGTTGGCTACCGTCTTTTTGACGAGGGTCTGCAGAGAGAGCGGTAATGACAT
Warthog MGF 360 13L (AY261366.1:27277-28338)
SEQ ID No. 103
CTATAGTATATTATGAGAATATTCCCAAATGATGGATAAGTTTTTTGATTTATAATCCTTTAATAAACTGCTTATTTCTTCGG
GGTCCTTTAAGTTTAGTGGCAAGGAAGCATCTGAGCTGTAAATATCCAAAGTCAAACTATGGCTTAGAAAATGATAACCTTTT
TGTTCCGCTATAGCACGACCCTCTTCAAAGGCATTACCACCCAAATCTATACAGAAAAATATATTACCGATGTTATAATATTG
TACTGAAGTAAGCATAGCTTGGTTGATGTTGCCCCCCAGGGCGTAACAATAATATATTGTTAATGGATTGTTATCCTTGGTAG
AAGCCAGACATATCATGTCATGGACGTCTATTTGGACTTTTTCCTTGTGGTACATCTCATGAAGCTCATATATTTTGTTATAA
TACAGGAGACATTTTAATCGCCATTCATTAAGATCCGTATATTTCTCATCTAGAAAGCAAATGGCGTCCTTACACTCGTATTG
TACTGCTTTGGCGTACCAATACTTCACTAGTAAATCGTTTAACTCATCCGTTTCTTTTATTTCTATGAGCCCCCATAGTCTTT
TACAAATTAAGCCCCTTAATTGTATAGTAAATTTGTTTTCTATATTAGGATTATTCATAAATATCTCATGGCACAAAATAATA
CGGCCGCTGGTTTTATTGTGCATTATCTTGGTGAAAATACGGAAAATATCGTTGTCCTCTAGAGTTTCTTTGGCACCTAGCTG
TCTACACAACTCTCGGATGTGCTCCGTATTGATAGAAAGCAAACTATAGTTGATATGTGCGCCCCATTCTGTAAAGAGCTTTA
TCAGACTATAGTTGTTTTCCTTAACAGCTATTATTAATGCTATATGGAGGTCTATATCTTCTCCTAAAAATCCTGATTTTATT
TGTATTTGGCCACGATCCATACAAAGCTTGAGGGGGACATCATGCCACCATAGGCCACAGTGTTTCAGAATGCAGTGTTCATC
TGTCGACAAACACTGGCTGGCTACCGTCTTTTTGACGAGGGTCTGCAGAGAGAGCGGCAACGACAT
Warmbaths MGF 360 13L (AY261365.1:30498-31559)
SEQ ID No. 104
CTATAGTATATTATGAGAATATTCCCAAATGATGGATAAGTTTTTTGATTTATAACCCTTTAATAAACTGCTTATTTCTTCGG
GGTCCTTTAAGTTTAGTGGCAAGGAAGCATCTGAGCTGTAAATATCCAAAGCCAAACTATGGCTTAGAAAATGATAACCTTTT
TGTTCCGCTATGGCACGACCCTCTTCAAAGGCATTACCACCCAAATCTATACAGAAAAATATATTACCGATGTTATAATATTG
TACTGAAGTAAGCATAGCTTGGTTGATGTTGCCCCCCAGGGCGTAACAGTAATATATTGTTAATGGATTGTTATCCTTGGTAG
AAGCCAGACATATCATGTCATGGACGTCTATTTGGACTTTATCCTTGTGGTACATCTCATGAAGCTCATATATTTTGTTATAA
TACAGGAGACATTTTAATCGCCATTCATTAAGACCCGTATATTTCTCATCTAGAAAGCAAATGGCGTCCTTACACTCGTATTG
TACTGCTTTGGCGTACCAATACTTCACTAGTAAATCGTTTAACTCATCCGTTTCTTTTATTTCTATGAGCCTCCATAGTCTTT
TACAAATTAAGCCCCTTAATTGTATAGTAAATTTGTTTTCTATATTAGGATTATTCATAAATATCTCATGGCACAAAATAATA
CGGCCGCTGGTTTTATTGTGCATTATCTTGGTGAAAATACGGAAAATATCGTTGTCCTCTAGAGTTTTTTTGGCACCTAGCTG
TCTACACAACTCTCGGATGTGTTCCGTATTGATAGAAAGCAAACTATAGTTGATATGTGCGCCCCATTCTGTAAAGAGCTTTA
TCAGACTATAGTTGTTTTCCTTAACAGCTATTATTAATGCTACATGGAGATCTATATCTTCTCCTAAAAATCCTGATTTTATT
TGTATTTGGCCACGATCCATACAAAGCTTGAGGGGGACATCATGCCACCATAGGCCACAGTGTTTCAGAATGCAGTGTTCATC
TGTCGACAAACACTGGCTGGCTACCGTTTTTTTGACGAGGGTCTGCAGAGAGAGCGGCAACGACAT
Pretorisuskop/96/4 MGF 360 13L (AY261363.1:31307-32368)
SEQ ID No. 105
CTATAGTATATTATGAGAATATTCCCAAATTATGGATAAGTTTTTTGATTTATAATCCTTTAAAAAACTGCTTATTTCTTCGG
GGTCCTTTAAGTTTAGTGGCAAGGAAGCATCTGAGCTGAAAATATCCAAAGCCAAACTATGGCTTAGAAAATTATAACCTTTT
TGTTCCGCTATGGCACGACCCTCTTCAAAGGCATTACCACCCAAATCTATACAGAAAAATATATTACCGATGTTATAATATTG
TATTGAAGTAAGCATAGCTTGGTTGATGTTGCCCCCCAGGGCGTAACAGTAATATATTGTTAATGGATTGTTATCCTTGGTAG
AAGCCAGACATATCATGTCATGGACGTCTATTTGGACTTTTTCCTTGTGGTACATCTCATGAAGCTCATATATTTTGTTAAAA
TACAGGAGACATTTTAATCGCCATTCATTAAGATCCGTATATTTCTCTTCTAGAAAGCAAATGGCGTCCTTACACTCGTATTG
TACTGCTTTGGCGTACCAATACTTCACTAGTAAATCGTTTAACTCATCCGTTTCTTTTATTTCTATGAGCCCCCATAGTCTTT
TACAAATTAAGCCCCTTAATTGTATAGTAAATTTGTTTTCTATATTAGGATTATTCATAAATATATCATGGCACAAAATAATA
CGGCCGCTGGTTTTATTGTGCATTATCTTGGTGAAAATACGGAAAACATCGTCGTCTTCTAGAGTTTCTTTGGCGCCTAGCTG
TCTACACAACTCTCGGATGTGCTCCGTATTAATAGAAAGCAAACTATAGTTGATATGTGCGCCCCATTCTGTAAAGAGCTTTA
TCAGACTATAGTTGTTTTCCTTAACAGCTATTATTAATGCTATATGGAGGTCTATATCCTCTCCTAAAAATCCTGATTTTATT
TGTATTTGGCCACGATCCATACAAAGCTTGAGGGGGACATCATGCCACCATAGGCCACAGTGTTTCAGAATGCAGTGTTCATC
TATCGACAAACACTGGCTGGCTACCGTCTTTTTGACGAGGGTCTGCAGAGAGAGCGGCAACGACAT
Mkuzi 1979 MGF 360 13L (AY261362.1:32286-33347)
SEQ ID No. 106
CTATAATATATTATGAGAATATTCCCAAATGATGGATAAGTTTTTTGATTTATAATCTTTTAATAAACTGCTTATTTCTTCGG
GGTCCTTTAAGTTTAATGGCAAGGAAGCATCTGAACTGTAAATATCCAAAGTCAAGCTATGGCTTAAAAAATTATAACCTTTT
TGTTCCGCTATGGCACGACCCTCTTCAAAGGCATTACCCCCCAAATCTATACAGAAAAATATATTACCGATGTTATAATATTG
TACTGAAGTGAGCATAGCTTGGTTGATGTTGCTCCCCAGGGCGTAACAGTAATATATTGTTAATAGATTGTTATCCTTGGCAC
AAGCCAGACATATCATGTCATGGACGTCTATTTGGACTTTTTTCTTGTGGTACATCTCATGAAGCTCATATATTTTGTTATAA
TACAGGAGACATTTTAATCGCCATTCGTTAAGATCCGTATATTTCTCATCTAGAAAGCAAATGGCGTTCTTACAAACGTATTG
TACTGCTTTGGCATACCAATACTTCACTAGTAAATCATTTAACTTGTCCGTTTCTTTTATTTCTATGAGCCCCCATAGTCTTT
TATAAATTAAGCCCCTTAATTGTATAACAAATTTGTTTTCTAAAATAGGATTATTCATAAAAATTTCATGGCACAAAATAATA
CTGCCGCTGGTTTTATTGTGCATTATCCTGGTGAAAATACGGAAAATATCGTTGTCCTCTAGAGTTTCTTTGGCGCCTAGCTG
TCTACACAACTCTCGGATGTGCTTCGTATTGATAGAAAGCAAACTATAGTTGATATTTGCGCCCCACTCTGTAAAGAGCTTTA
TCAGACTATAGTTGTTTTCCTTAACAGCTATTATTAATGCCACACGAAGGTCTATATCTTCTCCTAAAAATCCTGATTTTATT
TGTATTCGGCCACGATCCATACAAAGCTTGAGAGGAGCATCATGCCACCATAGGCCACAGTATTTCAAAATGCAGTGTTCATC
TATTGACAAACACTGGCTGGCTACCGTCTTTTTGACGAGGGTCTGCAGAGAGAGCGGCAACGACAT
L60 MGF 360 13L (NC_044941.1:25189-26250)
SEQ ID No. 107
CTATAATATATTATGAGAATATTCCCAAATGATGGATAAGTTTTTTGATTTATAATCTTTTAATAAACTGCTTATTTTTTCGG
GGTCCTTTAAGTTTAATGGCAAGGAAGCATCTGAACTGTAAATATCCAAAGTCAAGCTATGGCTTAAAAAATTATAACCTTTT
TGTTCCGCTATGGCACGACCCTCTTCAAAGGCATTACCACCCAAATCTATACAGAAAAATATATTACCGACGTTATAATATTG
TACTGAAGTGAGCATAGCTTGGTTGATGTTGCCCCCCAGGGCGTAACAGTAATATATTGTTAATAGATTGTTATCCTTGGCAC
AAGCCAGACATATCATGTCATGGACGTCTATTTGGACTTTTTTCTTGTGGTACATCTCATGAAGCTCATATATTTTGTTATAA
TACAGGAGACATTTTAATCGCCACTCGTTAAGATCCGTATATTTCTCATCTAGAAAGCAAATGGCGTTCTTACAAACGTATTG
TACTGCTTTGGCATACCAATACTTCACTAGTAAATCATTTAACTCGTCCGTTTCTTTTATTTCTATGAGCCCCCATAGTCTTT
TATAAATTAAGCCCCTTAATTGTATAACAAATTTGTTTTCTAACATAGGATTATTCATAAAAATTTCATGGCACAAAATAATA
CTGCCGCTGGTTTTATTGTGCATTATCCTGGTGAAAATACGGAAAATATCGTTGTCCTCTAGAGTTTCTTTGGCGCCTAGCTG
TCTACACAACTCTCGGATGTGCTTCGTATTGATAGAAAGCAAACTATAGTTGATATTTGCGCCCCACTCTGTAAAGAGCTTTA
TCAGACTATAGTTGTTTTCCTTAACAGCTATTATTAATGCCACACGAAGGTCTATATCTTCTCCTAAAAATCCTGATTTTATT
TGTATTCGGCCACGATCCATACAAAGCTTGAGAGGAGCATCATGCCACCATAGGCCACAGTATTTCAAAATGCAGTGTTCATC
TATTGACAAACACTGGCTGGCTACCGTCTTTTTGACGAGGGTCTGCAGAGAGAGCGGCAACGACAT
Benin 97/1 MGF 360 13L (NC_044956.1:24840-25901)
SEQ ID No. 108
CTATAATATATTATGAGAATATTCCCAAATGATGGATAAGTTTTTTGATTTATAATCTTTTAATAAACTGCTTATTTTTTCGG
GGTCCTTTAAGTTTAATGGCAAGGAAGCATCTGAACTGTAAATATCCAAAGTCAAGCTATGGCTTAAAAAATTATAACCTTTT
TGTTCCGCTATGGCACGACCCTCTTCAAAGGCATTACCACCCAAATCTATACAGAAAAATATATTACCGACGTTATAATATTG
TACTGAAGTGAGCATAGCTTGGTTGATGTTGCCCCCCAGGGCGTAACAGTAATATATTGTTAATAGATTGTTATCCTTGGCAC
AAGCCAGACATATCATGTCATGGACGTCTATTTGGACTTTTTTCTTGTGGTACATCTCATGAAGCTCATATATTTTGTTATAA
TACAGGAGACATTTTAATCGCCACTCGTTAAGATCCGTATATTTCTCATCTAGAAAGCAAATGGCGTTCTTACAAACGTATTG
TACTGCTTTGGCATACCAATACTTCACTAGTAAATCATTTAACTCGTCCGTTTCTTTTATTTCTATGAGCCCCCATAGTCTTT
TATAAATTAAGCCCCTTAATTGTATAACAAATTTGTTTTCTAACATAGGATTATTCATAAAAATTTCATGGCACAAAATAATA
CTGCCGCTGGTTTTATTGTGCATTATCCTGGTGAAAATACGGAAAATATCGTTGTCCTCTAGAGTTTCTTTGGCGCCTAGCTG
TCTACACAACTCTCGGATGTGCTTCGTATTGATAGAAAGCAAACTATAGTTGATATTTGCGCCCCACTCTGTAAAGAGCTTTA
TCAGACTATAGTTGTTTTCCTTAACAGCTATTATTAATGCCACACGAAGGTCTATATCTTCTCCTAAAAATCCTGATTTTATT
TGTATTCGGCCACGATCCATACAAAGCTTGAGAGGAGCATCATGCCACCATAGGCCACAATATTTCAAAATGCAGTGTTCATC
TATTGACAAACACTGGCTGGCTACCGTCTTTTTGACGAGGGTCTGCAGAGAGAGCGGCAACGACAT
Malawi Lil-20/1 (1983) MGF 360 13L (AY261361.1:28920-29980)
SEQ ID No. 109
TTAGAGTATATTATGAGAGTAATCCAAAATGATGGATAAATTTTTTGATTTATAATTTTTTAACAAACTGCTTATTTCTTCGG
GGTCCTTTAAGTTTAATGGCAAGGAAGCATCCGAGCTATAAATATTCAAAACCAAACTATGGCTTAGAAAATTATAACCTTTT
TGTCTCGCTATGGCACTACCCTCTTCAAAGGCATTACCACCCAAATCTATACAGAAATATATATTACCGATGTTATAATATTG
CACTGAAGTAAGCATAGCTTGGTTGATGTTGCCCCCCAGGGCATAACAGTAATATATTGTTAATGGATTGTTATCCCTGATAC
AAGCCAGGGATAACATTTCATTAACGTCTATTTGGATTTTTTCCTTGTGGTATATATCATGAAGCTCATATATTTTGTTATAA
TATAGGTAACATTTTAATCGCCATTCATTAAGATCCGTATATTTCTCATCCAGAAAGCAAATGGCATCTTTATAATCGTATTG
TACTGCTTTGGCGTACCAATATTTCACTAGTAATTCACTTAACTCGTCCGTTTCTTTTATTTCTATAAGCCCGCATAGTCTTT
TATAAATTAAGCGCCTTAATTGTGCAGCAAATTTGTTTTCTAAATTAGGATTATTTATAAATATCTCATGGCACAAAATAACA
CTGCCGCTGGTTTTATTATGCATTATCTTGTTGAAAATACAGAAAATATCGTAGTCCTCCAGAGTTTCTTTGGCGCCTAGCTG
TCTACACAAATCTCGGACGTGCTTCGTATTGATAGAAAGCAGACTATAGTTGATATTTGCGCCCAACTCTGTAAAAAGCTTTA
TTAGATTATAGTTGTTTTCCTTAACAGCTATTATTAATGCCACACAAAGGTCTATATCTTCTCCTAAAAATCCCGATTTTATT
TGTATTCGGCCACGATCCATATAAAGCTTGAGAGGGGCATCATGCCACCATAAGTCACAGTATTTCAAAATGTGTTCATCTAT
CGACAAACATGATGTACTGGCTACCGTCTTTTTGACGAGGGTTTGTAGAGAGAGCGGCGCCGACAT
Kenya 1950 MGF 360 13L (AY261360.1:34752-35812)
SEQ ID No. 110
TTAGAGTATTTTATGAGAGTAATCCAGAATGATGGATAAATTTTTTGATTTATAATTTTTTAATAAACTGCTTATTTCTTCGG
GAACCTTTAAGTTTAATGGCAAGGAAGCATCTGAGCTATAAATATTCAAAATTAAACTATGGCATAAAAAATTATAACCATTT
TGTCTCGCTATGGCACTACCCTCTTCAAAGGCATTACCACCCAAATCTATACAGAAATATATATTACCGATGTTATAATATTG
TACTGAAGTAAGCATAGCTTGGTTGATGTTGCCCCCCAGGGCATAACAATAATATATTGTTAATAGATTGTTATCCCTGATAC
AAGCCAGAGATAACATTTCATTGACGTCTATTTGGATTTTTTTCTTGTGGTATATATCATGAAGCTCATATATTTTGTTATAA
CATAGGTAACATTTTAATCGCCATTCATCAAGATCCGTATATTTTTCATCCAGAAAGCAAATGGCATCTTTATGATCGTATTG
TACTGCATTGGCGTACCAATATTTCACTAGTAATTCGCTTAACTCGTCCGTTTCTTTTATTTCTATAAGCCCGCATAATCTTT
TATAAATTAAGCGCCTTAATTGTGCAGCAAAGTTGTTTTCTAATTTAGGATTATTTATAAATATCTCATGGCACAAAATAATA
CTGCCGCTAGTTTTATTATGCATTATTTTATTGAAAATACAGAAAATATCGTAGTCGTCTAGAGTTTCTTTGGCGCCTAGCTG
TCTACACAAATCTCGGGCGTGCTTCGTATTGATAGAAAGCAGACTATAGTTGATATATGCGCCCCACTCTGTAAATAACTTTA
TCAGACTATAGTTATTTTCCTTAACAGCTATTATTAATGCCACACAAAGGTCTATATCTTCTCCTAGGAATCCCGATTTTATG
TATATTCGTCCACGATCTATGTAAAGCTTGAGGGGAGCATCATGCCACCATAAGCCACAGTATTTCAAAATGTGTTCATCTAT
CGACAAACATGATGTACTGGCTACCGTCTTTTTGACGAGGGTCTGCAGAGAGAGCGGCGACGACAT
Ken06.Bus MGF 360 13L (NC_044946.1:28644-29704)
SEQ ID No. 111
TTAGAGTATTTTATGAGAGTAATCCAGAATGATGGATAAATTTTTTGATTTATAATTTTTTAATAAACTGCTTATTTCTTCGG
GATCCTTTAAGTTTAATGGCAAGGAAGCATCTGAGCTATAAATATTCAAAACCAAACTATGGCTTAAAAAATTATAACCATTT
TGTCTCGCTATGGCACTACCCTCTTCAAAGGCATTACCACCCAAATCTATACAGAAATATATATTACCGATGTTATAATATTG
TACTGAAGTAAGCATAGCTTGATTGATGTTGCCCCCCAGGGCATAACAATAATATATTGTTAATAGATTGTTATCCCTGATAC
AAGCCAGAGATAACATTTCATTGACGTCTATTTGGATTTTTTCCTTGTGGTATATATCATGAAGCTCATATATTTTGTTATAA
CATAGGTAACATTTTAATCACCATTCATCAAGATCCGTATATTTTTCATCCAGAAAGCAAATGGCATCTTTATGGTCGTATTG
TACTGCATTGGCGTACCAATATTTCACTAGCAATTCGCTTAACTCGTCCGTTTCTTTTATTTCTATAAGCCCGCATAGTCTTT
TATAAATTAAGCGCCTTAATTGTGCAGCAAAGTTGTTTTCTAAATTAGGATTATTTATAAATATCTCATGGCACAAAATAATA
CTGCCGCTAGTTTTATTATGCATTATCTTGTTGAAAATACAGAAAATATCGTAGTCGTCTAGAGTTTCTTTGGCGCCTAGCTG
TCTACACAAATCTCGGGCGTGCTTCGTATTGATAGAAAGCAGACTATAGTTGATATATGCGCCCCACTCTGTAAATAACTTTA
TCATACTATAGTTGTTTTCCTTAACAGCTATTATTAATGCCACACAAAGGTCTATATCTTCTCCTAGGAATCCCGATTTTATG
TATATTCGCCCACGATCTATGTAAAGCTTGAGGGGAGCATCATGCCACCATAAGCCACAGTATTTCAAAATGTGTTCATCTAT
CGACAAACATGATGTACCGGCTACCGTCTTTTTGACGAGGGTCTGCAGAGAGAGCGGCGACGACAT

In an embodiment the attenuated ASFV of the invention comprises a functional version of 5 MGF 360 13L. Suitably the functional version of MGF 360 13L comprises the sequence of SEQ ID No. 100, 101, 102, 103, 104, 105, 106, 107, 108, 109, 110 or 111. Suitably the functional version of MGF 360 13L comprises a sequence having at least 70%, at least 80%, at least 90% or at least 95% identity with SEQ ID No. 100, 101, 102, 103, 104, 105, 106, 107, 108, 109, 110 or 111. Suitably the functional version of MGF 360 13L consists of the 10 sequence of SEQ ID No. 100, 101, 102, 103, 104, 105, 106, 107, 108, 109, 110 or 111.

MGF 360 14L gene sequences
Georgia 2007/1 MGF 360 14L (LR743116.1:32814-33887)
SEQ ID No. 112
TTAGTCTATAACTATAATTTCTGGATGGGCTGTAAGATACTCTTCGGCTCGTTTCAGATTTTTTGAAGTATATGTCTTTAGCA
TATCATATATTTCCTGGGGTTCGGTTACATCTAATACCAAGGTCACATCACGGCTGAAAAGCTGCTTTACTAAGAAAATGTTG
CTCAAGTTATACATATAAGCTTTGTGCGCAATGAGTTGTGCCCTATCAAAATCGGCAGCCCCCAAATCAATACAGAAAAACAT
GTTTAAAGTATTATTGTTATAGATAGAAAGATTCATGCCATAATCGAGACTAGCCCCCAACCTATGACAGTAATAAATGGCCG
CGTAATTTTTTTCCCGCAAGCAAGCAAATTTCATCATCAGATTAGGGCTGATGCAAATCTCTTTTTCACGACACAACTCGTGT
ATGTCAAAAATGTTATTAAAATAAAGGCTACAAGCTACCCGCCAATAGAGGTGATTTTTATGCCTTTTATAGAAATAGTGAAT
AGCCTTTGTAAAATTATGTCGTAATGCCAGGGCAAACCAAAACTTTGTTAATAGGTGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGCAGAACTCCTGCACAGGATTTTAGCCTGGCCACTTCTTTTAAAATTTCCAGAAGACGGGGTTCGGATACAGGCGTTAA
GCCTCCCAGTTCCGCACACAGCCGCTTTAGATACACGGCAGGAACACGTATAAGCCCATATTCAGGATTTGCGCCCCAATCCA
CAAATAAACGTATAAGTTCAAGATTATCGCTCTTCACGGCCTTTACTAGCGCCGCTTCGAGACAAAGATCATCCTCAGAAAAA
CACTGTAAATGTTTATACGAAAAAACTTGCTTACAATTGTTACATAGGTGAATAGGACCTAAATCCCACCACAAACCAAAACG
CTGCAACGTATAATCATAGTCACTTGAAAGATAATTGCATGCCACAACTTTTTTGGCCAACGTTTGTAAAGACAACAT
China/2018/AnhuiXCGQ MGF 360 14L (MK128995.1:31843-32916)
SEQ ID No. 113
TTAGTCTATAACTATAATTTCTGGATGGGCTGTAAGATACTCTTCGGCTCGTTTCAGATTTTTTGAAGTATATGTCTTTAGCA
TATCATATATTTCCTGGGGTTCGGTTACATCTAATACCAAGGTCACATCACGGCTGAAAAGCTGCTTTACTAAGAAAATGTTG
CTCAAGTTATACATATAAGCTTTGTGCGCAATGAGTTGTGCCCTATCAAAATCGGCAGCCCCCAAATCAATACAGAAAAACAT
GTTTAAAGTATTATTGTTATAGATAGAAAGATTCATGCCATAATCGAGACTAGCCCCCAACCTATGACAGTAATAAATGGCCG
CGTAATTTTTTTCCCGCAAGCAAGCAAATTTCATCATCAGATTAGGGCTGATGCAAATCTCTTTTTCACGACACAACTCGTGT
ATGTCAAAAATGTTATTAAAATAAAGGCTACAAGCTACCCGCCAATAGAGGTGATTTTTATGCCTTTTATAGAAATAGTGAAT
AGCCTTTGTAAAATTATGTCGTAATGCCAGGGCAAACCAAAACTTTGTTAATAGGTGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGCAGAACTCCTGCACAGGATTTTAGCCTGGCCACTTCTTTTAAAATTTCCAGAAGACGGGGTTCGGATACAGGCGTTAA
GCCTCCCAGTTCCGCACACAGCCGCTTTAGATACACGGCAGGAACACGTATAAGCCCATATTCAGGATTTGCGCCCCAATCCA
CAAATAAACGTATAAGTTCAAGATTATCGCTCTTCACGGCCTTTACTAGCGCCGCTTCGAGACAAAGATCATCCTCAGAAAAA
CACTGTAAATGTTTATACGAAAAAACTTGCTTACAATTGTTACATAGGTGAATAGGACCTAAATCCCACCACAAACCAAAACG
CTGCAACGTATAATCATAGTCACTTGAAAGATAATTGCATGCCACAACTTTTTTGGCCAACGTTTGTAAAGACAACAT
Tengani 62 MGF 360 14L (AY261364.1:26983-28056)
SEQ ID No. 114
TTAGTCTATAACTATAATTTCTGGATGGGCTGTAAGATACTCTTCAGCTCGTTTCAGATTTTTTGAAGTATATGACTTTAGCA
TATCATATATTTCCTGGGGTTCGGTTACATCTAATGCCAAGGTCACATCACGGCTGAAAAGCTGCTTTACTAAGAAAATGTTG
CTCAAGTTATACATATAAGCTTTGTGCGCAATGCGTTGTGCCCTATCAAAATCAGTAGCCCCCAAATCGATGCAGAAAAACAT
GTTTAAAGTATTATTGTTATAGATAGAAAGATTCATGCCATAATCGAGACTAGCCCCTAACCTATAACAGTAATAAATGGCCG
CATAATTTTTTTTCTGCAAGCAAGCAAATTTCATCATCAGATTAGGGCTGATGCAAATCTCTTTTTCACGACACAACTCGTGT
ATGTCAAAAATGTTATTAAAATAAAGGCTACAAGCTACCCGCCAATAGAGGTGATTTTTATGCCTTTTATAGAAATAGTGAAT
AGCCTTTGTAAAATTATGTCGTAATGCCAGGGCAAACCAAAACTTTGTTAATAGGTGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGCAGAACTCCTGCACAGGATTTTAGCCTGGCCACTTCTTTTAAAATTTCCAGAAGACGGGATTCGGATACAGGCGTTAA
GCCACCCAGTTCCGTGCACAGCCGCTTTAGATGCACGGCAGGAACACGTATAAGTCCATATTCAGGATTTGCGCCCCAATCCA
CAAATAAACGTATAAGCTCAAGATTATCGCTCTTCACGGCCTTTACTAGCGCCGCTTCAAGACAAAGATCATCCTCAGAAAAA
CACTGTACATGTTTATACGAAAAAATTTGCTTACAATTATTACATAGGTGAATAGGACCTAAATCCCACCACAAACCAAAACG
CTGCAACGTATAATCATAGTCACTTGAAAGATAATTGCATGCCACAACCTTTTTGGCCAATGTTTGTAAAGACAACAT
Warthog M GF 360 14L (AY261366.1:28495-29568)
SEQ ID No. 115
TTAGTCTATAACTATAATTTCTGGATGGGCTGTAAGATACTCTTCGGCTCGTTTCAGATTTTTTGAAGTATATGACTTTAGCA
TATCATATATTTCCTGGGGTTCGGTTACATCTAATGCCAAGGTCACATCACGGCTGAAAAGCTGCTTTACTAAAAAAATGTTG
CTCAAGTTATACATATAAGTTTTGTGCGCAATGCGTTGTGCCCTATCAAAATCAGTAGCCCCCAAATCGATGCAGAAAAACAT
GTTTAAAGTATTATTGTTATAGATAGAAAGATTCATGCCATAATCGAGACTAGCCCCTAACCTATAACAGTAATAAATGGCCG
CATAATTTTTTTTCCGCAAGCAAGCAAATTTCATCATCAGATTAGGGCTGATACAAATCTCTTTTTCACGACACAGCTCGTGT
ATGTCAAAAATGTTATTAAAATAAAGGCTACAAGCTACCCGCCAATAGAGGTGATTTTTATGCCTTTTATAAAAATAGTGAAT
AGCCTTTGTAAAATTATGTCGTAATGCCAGGGCAAACCAAAACTTTGTTAATAGGTGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGTAGAACTCCTGCACAGGATTTTAGCTTGGCCACTTCTTTTAAAATTTCCAGAAGACGGGGTTCGGATACAGGCGTTAA
GCCTCCCAGTTCCGTGCACAGCCGCTTTAGATGCACGGCAGGAACACGTATAAGTCCATATTCAGGATTTGCGCCCCAATCCA
CAAATAAACGTATAAGCTCAAGATTATCGCTCTTCACGGCCTTTACTAGCGCCGCTTCAAGACAAAGATCATCCTCAGAAAAA
CACTGTAAATGTTTATACGAAAAAATTTGCTTACAATTGTTACATAGGTGAATAGGACCTAAATCCCACCACAAACCAAAACG
CTGCAACATATAATCATAGTCACTTGAAAGATAATTGCATGCCACAACCTTTTTGGCCAACGTTTGTAAAGACAACAT
Warmbaths MGF 360 14L (AY261365.1:31747-32820)
SEQ ID No. 116
TTAGTCTATAACTATAATTTCTGGATGGGCTGTAAGATACTCTTCGGCTCGTTTCATATTTTTTGAAGTATATGCCTTTAGCC
TATCATATATTTCCTGGGGTTCGGTTACATCTAATGCCAAGGTCACATCACGGCTGAAAAGCTGCTTTACTAAAAGAATGTTG
CTCAAGTTATACATATAAGCTTTGTGCGCAATGCGTTGTGCCCTATCAAAATCAGTAGCCCCCAAATCGATGCAGAAAAACAG
GTTTAAAGAATTATTGTTATAGATAGAAAGATTCATGCCATAATCGAGACTAGCTCCCAACCTATAACAGTAATAAATGGCCG
CGTAATTTTTTTCCCGCAAGCAAGCAAATTTCATCATCAGATTAGGGCTAATGCAAATCTCTTTTTCACGACACAGCTCGTGT
ATGTCAAAAATGTTATTAAAATAAAGGCTACAAGCTACCCGCCAATAGAGGTGATTTTTATGCCTTTTATAGAAATAGTGAAT
AGCCTTTGTAAAATTATGTCGTAATGCCAGCGCAAACCAAAACTTTGTTAATAGGTGGTGCGCCGTATCCCCCATCAACGGAA
TTTTTGAACAGCTGTACATAACTGTGTCTAAAGTGGTTCTAGTTATGGTTTCCAAGAGTGGGTTATAACAAAACATGTCATAA
CCCAGTAGAACTCCTGCACAGGATTTTAGGTTGGCCACTTCTTTTAAAATTTCCAGAAGACGGGGTTCGGATACAGGTGTTAA
GCCTCCCAGTTCTGTGCACAGCCGCTTTAGATGCACGGTAGGAACACATATAAGTCCATATTCAGGATTTGCACCCCAATCCA
CAAATAAACGTATAAGCTCAAGATTATCCCTCTTCACGGCCTTTACTAGCGCCGCTTCAAGACAGAGATCATCCTCAGAAAAA
CACTGTAAATGTTTATACGAAAAAATTTGCTTACAATTGTTACACAGGTGAATAGGACCTAAATCCCACCACAAACCAAAACG
CTGCAACATATAATCATAGTCACTTGAAAGATAATTGCATGCCACAACCTTTTTGGCCAACGTTTGTAAAGACAACAT
Pretorisuskop/96/4 MGF 360 14L (AY261363.1:32525-33598)
SEQ ID No. 117
TTAGTCTATAACTATAATTTCTGGATGGGCTGTAAGATACTCTTCGGCTCGTTTCAGATTTTTTGAAGTATATGACTTTAGCC
TATCATATATTTCCTGGGGTTCGGTTACATCTAATGCCAAGGTCACATCACGGCTGAAAAGCTGCTTTACTAAGAAAATGTTG
CTCAAGTTATACATATAAGCTTTGTGCGCAATGTGTTGTGCCCTATCAAAATCAGTAGCCCCCAAATCGATGCAGAAAAACAT
GTTTAAAGTATTATTGTTATAGATAGAAAGATTCATGCCATAATCGAGACTAGCCCCTAACCTATAACAGTAATAAATGGCCG
CATAATTTTTTTTCTGCAAGCAAGCAAATTTCATCATCAGATTAGGGCTGATGCAAATCTCTTTTTCACGACACAGCTCGTGT
ATGTCAAAAATGTTATTAAAATAAAGGCTACAAGCTACCCGCCAATAGAGGTGATTTTTATGCCTTTTATAGAAATAGTGAAT
AGCCTTTGTAAAATTATGTCGTAATGCCAGGGCAAACCAAAACTTTGTTAATAGGAGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGTAGAACTCCTGCACAGGATTTTAGCTTGGCCACTTCTTTTAAAATTTCCAGAAGACGGGGTTCGGTTACAGGCGTTAA
GCCTCCCAGTTCCGTGCACAGCCGCTTTAGATGCACGGCAGGAACACGTATAAGTCCATATTCAGGATTTGCGCCCCAATCCA
CAAATAAACGTATAAGCTCAAGATTATCGCTCTTCACGGCCTTTACTAGCGCCGCTTCAAGACAAAGATCATCCTCAGAAAAA
CACTGTAAATGTTTATACGAAAAAATTTGCTTACAATTGTTACACAGGTGAATAGGACCTAAATCCCACCACAAACCAAAACG
CTGCAACGTATAATCATAGTCACTTGAAAGATAATTGCATGCCACAACCTTTTTGGCCAACGTTTGTAAAGACAACAT
Mkuzi 1979 MGF 360 14L (AY261362.1:33547-34620)
SEQ ID No. 118
TTAGTCTATAACTATAATTTCTGGATGGGCTGTAAGATACTCTTCGGCCCGTTTCATATTTTTTGAAGTATATGTCTTTAGCA
TATCATATATTTCCTGGGGTTCGGTTACATCTAATACCAAGGTCACATCACGGCTGAAAAGCTGCTTTACTAAGAAAATGTTG
CTCAAGTTATACATATAAGCTTTGTGCGCAATGAGTTGTGCCCTATCAAAATCGGCAGCCCCCAAATCAATACAGAAAAACAT
GTTTAAAGTATTATTGTTATAGATAGAAAGATTCATGCCATAATCGAGACTAGCCCCCAACCTATGACAGTAATAAATGGCCG
CGTAATTTTTTTCCCGCAAGCAAGCAAATTTCATCATCAGATTAGGGCTAATGCAAATCTCTTTTTCACGACACAGCTCGTGT
ATGTCAAAAATGTTATTAAAATAAAGGCTACAAGCTACCCGCCAATAGAGCTGATTTTTATGCCTTTTATAAAAATAGTGGAT
AGCCTTTGTAAAATTATGTCGTAATGCCAGGGCAAACCAAAACTTTGTTAATAGGAGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGTAGAACTCCTGCACAGGATTTTAGGTTGGCCACTTCTTTTAAAATTTCCAGAAGACGGGGTTCGGATACAGGCGTTAA
GCCTCCCAGTTCCGCACACAGCCGCTTTAGATACACGGCAGGAACACGTATAAGCCCATATTCAGGATTTGCGCCCCAATCCA
CAAATAAACGTATAAGTTCAAGATTATCGCTCTTCACGGCCTTTACTAGCGCCGCTTCAAGACAAAGATCATCCTCAGAAAAA
CACTGTAAATGTTTATACGAAAAAACTTGCTTACAATTGTTACATAGGTGAATAGGACCTAAATCCCACCACAAACCAAAACG
CTGCAACGTATAATCATAGTCACTTGAAAGATAATTGCATGCCACAACTTTTTTGGCCAACGTTTGTAAAGACAACAT
L60 MGF 360 14L (NC_044941.1 26422-27495)
SEQ ID No. 119
TTAGTCTATAACTATAATTTCTGGATGGGCTGTAAGATACTCTTCGGCTCGTTTCATATTTTTTGAAGTATATGTCTTTAGCA
TATCATATATTTCCTGGGGTTCGGTTACATCTAATACCAAGGTCACATCACGGCTGAAAAGCTGCTTTACTAAGAAAATGTTG
CTCAAGTTATACATATAAGCTTTGTGCGCAATGAGTTGTGCCCTATCAAAATCGGCGGCCCCCAAATCAATACAGAAAAACAT
GTTTAAAGTATTATTGTTATAGATAGAAAGATTCATGCCATAATCGAGACTAGCCCCCAACCTATGACAGTAATAAATGGCCG
CGTAATTTTTTTCCCGCAAGCAAGCAAATTTCATCATCAGATTAGGGCTAATGCAAATCTCTTTTTCACGACACAGCTCGTGT
ATGTCAAAAATGTTATTAAAATAAAGGCTACAAGCTACCCGCCAATAGAGCTGATTTTTATGCCTTTTATAAAAATAGTGGAT
AGCCTTTGTAAAATTATGTCGTAATGCCAGGGCAAACCAAAACTTTGTTAATAGGAGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGTAGAACTCCTGCACAGGATTTTAGGTTGGCCACTTCTTTTAAAATTTCCAGAAGACGGGGTTCGGATACAGGCGTCAA
GCCTCCCAGTTCCGCACACAGCCGCTTTAGATACACGGCAGGAACACGTATAAGCCCATATTCAGGATTTGCGCCCCAATCCA
CAAATAAACGTATAAGTTCAAGATTATCGCTCTTCACGGCCTTTACTAGCGCCGCTTCAAGACAAAGATCATCCTCAGAAAAA
CACTGTAAATGTTTATACGAAAAAACTTGCTTACAATTGTTACATAGGTGAATAGGACCTAAATCCCACCACAAACCAAAACG
CTGCAACGTATAATCATAGTCACTTGAAAGATAATTGCATGCCACAACTTTTTTGGCCAACGTTTGTAAAGACAACAT
Benin 97/1 MGF 360 14L (NC_044956.1:26073-27146)
SEQ ID No. 120
TTAGTCTATAACTATAATTTCTGGATGGGCTGTAAGATACTCTTCGGCTCGTTTCATATTTTTTGAAGTATATGTCTTTAGCA
TATCATATATTTCCTGGGGTTCGGTTACATCTAATACCAAGGTCACATCACGGCTGAAAAGCTGCTTTACTAAGAAAATGTTG
CTCAAGTTATACATATAAGCTTTGTGCGCAATGAGTTGTGCCCTATCAAAATCGGCGGCCCCCAAATCAATACAGAAAAACAT
GTTTAAAGTATTATTGTTATAGATAGAAAGATTCATGCCATAATCGAGACTAGCCCCCAACCTATGACAGTAATAAATGGCCG
CGTAATTTTTTTCCCGCAAGCAAGCAAATTTCATCATCAGATTAGGGCTAATGCAAATCTCTTTTTCACGACACAGCTCGTGT
ATGTCAAAAATGTTATTAAAATAAAGGCTACAAGCTACCCGCCAATAGAGCTGATTTTTATGCCTTTTATAAAAATAGTGGAT
AGCCTTTGTAAAATTATGTCGTAATGCCAGGGCAAACCAAAACTTTGTTAATAGGAGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGTAGAACTCCTGCACAGGATTTTAGGTTGGCCACTTCTTTTAAAATTTCCAGAAGACGGGGTTCGGATACAGGCGTCAA
GCCTCCCAGTTCCGCACACAGCCGCTTTAGATACACGGCAGGAACACGTATAAGCCCATATTCAGGATTTGCGCCCCAATCCA
CAAATAAACGTATAAGTTCAAGATTATCGCTCTTCACGGCCTTTACTAGCGCCGCTTCAAGACAAAGATCATCCTCAGAAAAA
CACTGTAAATGTTTATACGAAAAAACTTGCTTACAATTGTTACATAGGTGAATAGGACCTAAATCCCACCACAAACCAAAACG
CTGCAACGTATAATCATAGTCACTTGAAAGATAATTGCATGCCACAACTTTTTTGGCCAACGTTTGTAAAGACAACAT
Malawi Lil-20/1 (1983) MGF 360 14L (AY261361.1:30193-31266)
SEQ ID No. 121
TTAGTCTATAACTACAATTTCTGGATGGGCTGTAAAATACTCTTCGGCTTGTTTTAGATTTTTTGACGTATATGTATTTAGCA
TATCATATATTGCCTGGGGCTCAGTTAGATCTAATATCAAGCTCACATCACGGCTGAAAAGCTGTTTTACTAAAAAAAGGTTG
CTCAAGTTATACATATAAACTCTACGCGCAATGAGTCGCGCCCTATCAAAGTTAGTAGCCCCCAAATCAATGCAGAAAAATAG
GTTTAAAGTATTATTGTTATAGATAGATAGATTCATGCCATAATCAAGACTAGCCCCTAACATATGACAGTAATAAATGGCTG
CATAATTTTCTTCCCGCAAGCAAGCAAATTTCATCATCAGATTAGGGCTAATGCAAATCTCTTTTTTACAACACAGCTCATGC
AAGTCAAAAATGTTGTTAAAATAAAGGCTACAAGTCAGTCGCCAATAGAGCTGATTTTTATGCTTTTCGTAGAAATAGTGAAT
AGCTTTTGTAAAATTATGTCGTAATGCCAGGGCAAACCAAAACTTTGATAATAGGAGGTGCGCCGTATCCCCCGTCAACGGAA
TTTTTGAACGGGTGTACATAACTGTGTCTAAGGTGGTTCTAGTCACGGTTTCCAAGAGTGGATTATGACAAAACATGTCATAA
CCCAGTAGAACTCCTGCACAGGATTTCAGATCGGCCACTTCTTTTAAAATCTCCAGAAGACGAGATTCGGATACAGGTGTTAA
GCCTCCTAGTTCCATGCACAGCCGCTTTAGATGCACGGCAGGAACACGTATAAGTCCATATTCAGGATTTGCGCCCCAATCCA
CAAACAATCGTATAAGCTCAAGATTATCGCTCTTCACGGCCTTTACTAGCGCCGCTTCAAGACAAAGATCATCCTCAGAAAAA
CACTGTAAATGTTTATATGAAAAAATTTGCTTACATGTATTACATAAGTGAATAGGACCTAAATCCCACCACAAACCAAAACG
CTGCAGCGTATAATCATAATCACTTGAAAGATAATTGCATGCCACAACTTTTTTGGCCAGCGTTTGTAAAGACAACAT
Kenya 1950 MGF 360 14L (AY261360.1:36121-37194)
SEQ ID No. 122
TTAGTCTATAACTACAATTTCTGGATGGGCTGTAAAATACTCTTCGGCTCGTTTTAGATTTTTTGACGTATATGTCTTTAGCA
TATCATATATTGCCTGGGGTTCGGTTATATTTAATACCAAGCTCGCATCACGGCTGAAAAGCTGTTTTACTAAAAAAATGTTG
CTCAAGTTATACATATAAACTCTATGCGCAATGAGTCGCGCTCTATCAAAGTTAGTAGCCCCCAAATCAATACAGAAAAATAG
GTTTAAAGTATTATTGTTATAGATAGATAGATTCATGCCATAATCGGGACTAGCCCCCAACATATGACAGTAATAAATGGCTG
CATAATTTTCTTCCCGCAGGCAAGCAAATTTCATCATTAGATTAGGGCTAATGCAAATCTCTTTTTTAGAACACAGCTCATGC
AAGTCAAAAATATTATTAAAATAAAGGCTACAAGCCAGCCGCCAATAGAGCTGATTTTTATGCTTTTCATAGAAATAGTGAAT
CGCTTTTGTAAAATTATGTCGTAATGCCAGGGCAAACCAAAACTTCGTTAATAGGAGGTGCGCCGTATCCCCCGTCAACGGAA
TTTTTGAACGGGTGTACATAACGGTGTTTAAAGTGGTTCTAGTCACGGTTTCCAAGAGTGGATTATGACAAAACATGTCATAA
CCCAGTAGAACTCCTGCACAGGATTTCAGATCGGCCACTTCTTTTAAAATTTCCAGAAGACGAGATTCGGAGACAGGTGTTAA
GCCTCCTAGTTCTGTGCACAGCCGTTTTAGATCCACGGCAGGAACACGTATAAGTCCATATTCAGGATTTGCGCCCCAATCCA
CAAACAATCGTATAAGCTCAAGATTATCGCTCTTCACGGCCTTTACTAGCGCCGCGTCAAGACAAAGATCATCCTCAGAAAAA
CACTGCAAATGTTGATACGAAAAAATTTGCTTACATGTATTACATAAGTGAATAGGACCTAAATCCCACCACAAACCAAAACG
CTGCAGCGTATAATCATAGTCACTTGAAAGATAATTGCATGCCACAACCTTTTTGGCCAACGTTTGTAAAGACAACAT
Ken06.Bus MGF 360 14L (NC_044946.1:29917-30990)
SEQ ID No. 123
TTAGTCTATAACGACAATTTCTGGATGGGCTGTAAAATACTCTTCGGCTCGTTTTAGATTTTTTGACGTATATGTCTTTAGCA
TATCATATATTGCCTGGGGTTCGGTTATATTTAATACCAAGCTCGCATCACGGCTGAAAAGCTGTTTTACTAAAAAAATGTTG
CTCAAGTTATACATATAAACTCTATGCGCAATGAGTCGCGCTCTATCAAAGTTAGTAGCCCCCAAATCAATGCAGAAAAATAG
GTTTAAAGTATTATTGTTATAGATAGATAGATTCATGCCATAATCGGGACTAGCCCCCAACATATGACAGTAATAAATGGCTG
CATAATTTTCTTCCCGCAGGCAAGCAAATTTCATCATTAGATTAGGGCTAATGCAAATCTCTTTTTTAGAACACAGCTCATGC
AAGTCAAAAATGTTATTAAAATAAAGGCTACAAGTTAGCCGCCAATACAGCTGATTTTTATGTTTTTCGTAGAAATAGTGAAT
CGCTTTTGTAAAATTATGCCGTAATGCCAGGGCAAACCAAAACTTCGTTAATAGGAGGTGCGCCGTATCCCCCGTCAACGGAA
TTTTTGAACGGGTATACATAACTGTGTTTAAAGTGGTTCTAGTCACGGTTTCCATAAGTGGATTATGACAAAACATGTCATAA
CCCAGTAGAACTCCTGCACAGGATTTCAGATCGGCCACTTCTTTTAAAATTTCCAGAAGACGAGATTCGGAGACAGGTGTTAA
GCCTCCTAGTTCCGTGCACAGCCGTTTTAGATGCACGGCAGGAACACGTATAAGTCCATATTCAGGATTTGCGCCCCAATCCA
CAAACAATCGTATAAGCTCAAGATTATCGCTCTTCACGGCCTTTACTAGCGCCGCGTCAAGACAAAGATCATCCACAGAAAAA
CACTGCAAATGTTGATACGAAAAAATTTGCTTACATGTATTACATAAGTGAATAGGACCTAAATCCCACCACAAACCAAAACG
CTGCAGCGTATAATCATAGTCACTTGAAAGATAATTGCACGCCACAACTTTTTTGGCCAACGTTTGTAAAGACAACAT

In an embodiment the attenuated ASFV of the invention comprises a functional version of 20 MGF 360 14L. Suitably the functional version of MGF 360 14L comprises the sequence of SEQ ID No. 112, 113, 114, 115, 116, 117, 118, 119, 120, 121, 122, or 123. Suitably the functional version of MGF 360 14L comprises a sequence having at least 70%, at least 80%, at least 90% or at least 95% identity with SEQ ID No. 112, 113, 114, 115, 116, 117, 118, 119, 120, 121, 122, or 123. Suitably the functional version of MGF 360 14L consists of the 25 sequence of SEQ ID No. 112, 113, 114, 115, 116, 117, 118, 119, 120, 121, 122, or 123.

Georgia 2007/1 MGF 360 14L
SEQ ID No. 124
ATGTCTACTCCACTTTCTCTACAGACTCTTGTTAAAAAAGTGCTGGCCACACAGCACATATCTAAAGAACACTACTTTATTTT
GAAATATTGTGGTTTATGGTGGCATGAAGCGCCGATTACGATTTGCATTGATGAGGATAGCCAAATATTGATAAAATCGGCAA
GCTTCAAAGAAGGCTTATCTTTAGATATCGCATTAATGAAAGTCGTGCAAGAAAATAACCATGATTTAATAGAGTTGTTTACC
AAGTGGGGTGCAGATATCAACTCTAGCTTAGTTACTGTTAATACGGAGTATACCCGGAACCTTTGTCAGAAATTAGGCGCAAA
GGAAGCTTTGAATGAAAGGGATATTTTACAAATATTTTATAAAACACGTCATCTTAAAACTAGCAGTAATATTATTTTATATA
ATGAATTGTTTTCTAATAATCTCCTTTTCCAAAATATAGAGAGATTGAGTTTAATAGTTTATAGGGGCTTGAAAAACTTATCA
ATCAACTTTATATTGGATGATATTTCATTTAGCGAAATGTTAACTAGATACTGGTATAGTATGGCGATATTATATAACCTTAC
TGAAGCCATCCAATATTTTTATCAACGATATAGGCATTTTAAAGATTGGCGGCTTATATGTGGGCTTTCTTTTAACAATTTGT
CTGACCTTCATGAAGTATATAACTTAGAGAAGACGGATATAGACATTGATGAAATGATGAAGTTGACCTGTAGTACGTATGAT
GGTAATTATTCGACTATTTATTATTGTTTTATGTTGGGGGCTGACATCAATCGGGCAATGTTAACCTCGGTAATAAACTTTCA
TATTGGTAACTTGTTCCTTTGTATAGATTTAGGAGCTGATGCTTTCGAAGACAGCATGGAACTAGCAAAACAAAAGAATAATA
ATATATTAGTAGAAATATTATCATTTAAAAATTATTATAGTTCAAATACCTCTCTTTTATCAATAAAAACGACAGATCCGGAA
AAAATTAATGCCTTATTAGATGAAGAAAAGTATGAGTCAAAAAATATGTTAATGTATGAAGAATTATCTCATTGA
China/2018/AnhuiXCGQ MGF 360 14L
SEQ ID No. 125
ATGTCTACTCCACTTTCTCTACAGACTCTTGTTAAAAAAGTGCTGGCCACACAGCACATATCTAAAGAACACTACTTTATTTT
GAAATATTGTGGTTTATGGTGGCATGAAGCGCCGATTACGATTTGCATTGATGAGGATAGCCAAATATTGATAAAATCGGCAA
GCTTCAAAGAAGGCTTATCTTTAGATATCGCATTAATGAAAGTCGTGCAAGAAAATAACCATGATTTAATAGAGTTGTTTACC
AAGTGGGGTGCAGATATCAACTCTAGCTTAGTTACTGTTAATACGGAGTATACCCGGAACCTTTGTCAGAAATTAGGCGCAAA
GGAAGCTTTGAATGAAAGGGATATTTTACAAATATTTTATAAAACACGTCATCTTAAAACTAGCAGTAATATTATTTTATATA
ATGAATTGTTTTCTAATAATCTCCTTTTCCAAAATATAGAGAGATTGAGTTTAATAGTTTATAGGGGCTTGAAAAACTTATCA
ATCAACTTTATATTGGATGATATTTCATTTAGCGAAATGTTAACTAGATACTGGTATAGTATGGCGATATTATATAACCTTAC
TGAAGCCATCCAATATTTTTATCAACGATATAGGCATTTTAAAGATTGGCGGCTTATATGTGGGCTTTCTTTTAACAATTTGT
CTGACCTTCATGAAGTATATAACTTAGAGAAGACGGATATAGACATTGATGAAATGATGAAGTTGACCTGTAGTACGTATGAT
GGTAATTATTCGACTATTTATTATTGTTTTATGTTGGGGGCTGACATCAATCGGGCAATGTTAACCTCGGTAATAAACTTTCA
TATTGGTAACTTGTTCCTTTGTATAGATTTAGGAGCTGATGCTTTCGAAGACAGCATGGAACTAGCAAAACAAAAGAATAATA
ATATATTAGTAGAAATATTATCATTTAAAAATTATTATAGTTCAAATACCTCTCTTTTATCAATAAAAACGACAGATCCGGAA
AAAATTAATGCCTTATTAGATGAAGAAAAGTATGAGTCAAAAAATATGTTAATGTATGAAGAATTATCTCATTGA
Tengani 62 MGF 360 14L
SEQ ID No. 126
ATGTCTACTCCACTTTCTCTACAGACTCTTGTTAAAAAAGTGCTGGCCACACAGCACGTATCTAAAGATCACTACTTTATTTT
AAAATATTGTGGTTTGTGGTGGCATAAGGCCCCGATTACGATTTGCATTGATGAGGATAGCCAAATATTGATAAAATCGGCAA
GCTTTAAAGAAGGCTTACCTTTAGATATGGCATTAATGAAAGCTGTACAGGAAAACAACTATGAAATGATCATGTTGTTTACT
GAGTGGGGTGCAGACATTAACTCTAGCTTAGTCACTGTTAATACGGAGCGTACCCGGAACCTTTGTCGAGAATTAGGCGCGAA
GGAAATTTTGAATGAAAAAGAAATTTTAGAAATATTTTATAAAACATGTCATATTAAAACTAGCAGTAATTTTATTCTATGCC
ATGAATTGTTATCCAATAATCCCCTTTTCCAAAATATAGAGAGTTTGAGTTTAATAGTTTATAGGGGCTTGAAAAACTTATCA
ATTAACTTTATATTGGATGATATTTCATTTAGCGAAATGTTAACTAGATACTGGTATAGCTGGGCGATATTATATAACCTTAC
TGAAGCTATTCAATATTTCTATCAAACATACAAGCATTTTAAAGATTGGCGGCTAATATGTGGACTTGCTTATAATAACGTGT
TTGATCTTCATGAAATTTATAACATAGAAAAGACTGATTTAGATATTGATGAAATGATGCAGTTGGCTTGTATGTATGATAGT
AATTATTCAACTATTTATTATTGTTTTATGTTGGGGGCTAATATCAATCAGGCAATGTTAACCTCGGTAATAAACCATTATAT
TAATAACTTGTTCCTTTGTATAGATTTGGGAGCTGACGCCGTCAAAGACAGCATGGAACTAGCAAAACAAAAGAATTATAATA
TATTAGTAAAAATATTAACATTTAAAAATTATAGTCCAGACTCTTCTCTTTTATCATTAAAAACGACAGATCCGGAAAAAATT
AATGCCTTACTAGATGAAGAAAAGTATGAGTCGAAAAATATGTTAATGTATGAAGAATCATCTCGTTGA
Pretorisuskop/96/4 MGF 360 14L
SEQ ID No. 127
ATGCCTTCTCCACATTCTCTACAGACTCTTGCTAAAAAAATACTGGCTACACAGCAGATATCTACAGATCACTACTTTATTTT
AAAATATTGTGGTTTATGGTGGCATGGAGCTCCAATTATGCTTTCTACTAATGAGGATAATCAATTGATGATAAAATCGGCAA
GCTTCAAAGAAGGCTTATCTTTAGATTTGGCATTAATGAAAGTCGTGCAAGAAAACAATCATGATTTAATAAAGCTGTTTACC
GAGTGGGGTGCAGACATCAACTCTAGCTTCGTCACTGTTAATATGGAGTGTACCCGAAACCTCTGTCGGGAATTAGGCGCGAA
GGAAGCTTTGAATGAAAGGGATATTTTACAAATATTTTATAAAACACGTGATATTAAAACTAGCAGTCATGTTATTTTATGCC
ATGAATTATTATCCAATAACCCCCTTTTCCAGAATATAGAGAGAATGCGTTCAATAATTTATAGGAGTCTGGAAAAACTATCA
ATCAACTTTATATTGGATGATATTTCATTTAGTGAAATGTTAACTAGACACTGGTATGGGTTGGCGATATTATATAACCTAAC
TGAAGCTATCCAATATTTCTATGAAAAATACAAGCATTTTAAAAATTGGCGGCTAATATGTGGACTTTCTTTTAACAATCTGT
CTGATCTTTATGAAATATATAATTTAGAGAAGGTTGATATGAACATTGATGAAATGATGTATCTGGCCTGTAGTATATATGAT
GGTAATTATTCAACTATTTATTATTGTTTTGTATTGGGAGCTGATATCAATCAGGCAATGTTAACCTCAGTAATAAACCATTG
TATTGGTAACTTGTTCCTTTGTATAGATTTAGGGGCTGATGCTTTCGAAGACAGCATGGAACTAGCAAAGCAAAAAAATGATA
ATATTTTTATTAGTATATTATCATTTAAAAATTACAGTCCAGACTCTTCTCTTTTGTCATTAAAAATGACAGATCCGGAAAAA
ATTAATGCCTTACTAGATGAAGAAAAGTATGAGTCAAAAAATATGTTAATGTTTGA
Mkuzi 1979 MGF 360 14L
SEQ ID No. 128
ATGCCTACTCCACTTTCTCTACAGACTCTTGCTAAAAAAGTACTGGCCACACAGTATATATCCATAGATTATTACTTTATTTT
GAAATATTGTGGTTTATGGTGGCATGGAGCCCCTATTATGATTTCCATTGATAAGAATAAAAGCCAAATACTGATAAAATCGG
CAAGCTTCAAAGAAGGCTTATCTTTAGATATCGCATTAATGAAAGCTGTGCAGGAAAACAATCATGATTTAATAAAGCTGTTT
ACCGAGTGGGGTGCAGACATCAACTCTAGCTTAGTCACTGTTAATACGGAGTGTACCCGAAACCTCTGTCGGGAATTAGGCGC
GAAGGAAGCTTTGAATGAAAGGGATATTTTACAAATATTTTATAAAACACGTGATATTAAAACTAGCAGTAATGTTATACTAT
GCCATGAATTGTTATCCAATAACCCCCTTTTCCAAAATATAGAGAGAATGCGTTCAATAATTTATAGGAGTCTGGAAAAACTA
TCAATTAACTTTATATTGGATGATATTTCATTTAGCGAAATGTTAACTAGACACTGGTATAGGTTGGCGATATTATATAACCT
AACTGAAGCTATCCAATATTTCTATGAAAAATACAAGCATTTTAAAGATTGGCGGCTAATATGTGGGCTTTCTTTTAACAATC
TGTCTGACCTTCATGAAATATATAACTTAGAGAAGGTTGATATGGACATTGATATAATGATGTATCTGGCCTGTAGTACGTAT
GATGGTAATTATTCAACTATTTATTATTGTTTTGTATTGGGGGCTGATATCAATCAGGCAATGTTAACCTCGGTAATAAACCA
TCGTATTGGTAACTTGTTCCTTTGTATAGATTTAGGGGCTGATGCCTTCGAAGACAGTATGAAACTAGCAAAACAAAAGAATC
ATAATATATTAGTACATATATTATCGTTTAAAAACTATAGTCCAGACTTCTCCCTTTTATCAATAAAAACGACAGATCCAGAG
AAAATTAATGCCTTACTAGATGAAGAAAAGTATGAGTCAAAAAATATGTTAATGTATGATGAATTTGACGCCTGTACTAATAA
TGTTTAA
Warmbaths MGF 360 14L
SEQ ID No. 129
ATGCCTACTCCACTTTCTCTACAGACTCTTGCTAAAAAAGTACTGGCCACACAGTATATATCCATAGATTATTACTTTATTTT
GAAATATTGTGGTTTATGGTGGCATGGAGCCCCTATTATGATTTCCATTGATAAGAATAAAAGCCAAATACTGATAAAATCGG
CAAGCTTCAAAGAAGGCTTATCTTTAGATATCGCATTAATGAAAGCTGTGCAGGAAAACAATCATGATTTAATAAAGCTGTTT
ACCGAGTGGGGTGCAGACATCAACTCTAGCTTAGTCACTGTTAATACGGAGTGTACCCGAAACCTCTGTCGGGAATTAGGCGC
GAAGGAAGCTTTGAATGAAAGGGATATTTTACAAATATTTTATAAAACACGTGATATTAAAACTAGCAGTAATGTTATACTAT
GCCATGAATTGTTATCCAATAACCCCCTTTTCCAAAATATAGAGAGAATGCGTTCAATAATTTATAGGAGTCTGGAAAAACTA
TCAATTAACTTTATATTGGATGATATTTCATTTAGCGAAATGTTAACTAGACACTGGTATAGGTTGGCGATATTATATAACCT
AACTGAAGCTATCCAATATTTCTATGAAAAATACAAGCATTTTAAAGATTGGCGGCTAATATGTGGGCTTTCTTTTAACAATC
TGTCTGACCTTCATGAAATATATAACTTAGAGAAGGTTGATATGGACATTGATATAATGATGTATCTGGCCTGTAGTACGTAT
GATGGTAATTATTCAACTATTTATTATTGTTTTGTATTGGGGGCTGATATCAATCAGGCAATGTTAACCTCGGTAATAAACCA
TCGTATTGGTAACTTGTTCCTTTGTATAGATTTAGGGGCTGATGCCTTCGAAGACAGTATGAAACTAGCAAAACAAAAGAATC
ATAATATATTAGTACATATATTATCGTTTAAAAACTATAGTCCAGACTTCTCCCTTTTATCAATAAAAACGACAGATCCAGAG
AAAATTAATGCCTTACTAGATGAAGAAAAGTATGAGTCAAAAAATATGTTAATGTATGATGAATTTGACGCCTGTACTAATAA
TGTTTAA
Ken05/Tk1 MGF 360 14L
SEQ ID No. 130
ATGCCTACACCGTCTTCTTTACAAGTCCTTGTCAAAAGGGTGTTGGACTACCAGCATCATGTATCTGAAGATGATTATTGTAT
TTTACAGCATTGTGGGTTGTGGTGGCATGGAAGTCCAATTATGTTTTTTACTAATGAAAATCATCAAAGGATGATAAAATCAG
CAAGCTTTAAAGATGGTTTAGAAATAAATCTCGCATTAATGAAGGCCGTACAGGAAAACAATTGTATCTTAATAGAGCTGTTT
ACCGAATGGGGTGCAGACATTAATTATGGATTGATTACTGTCAATACGGAATATACCCGAAACCTATGTCGAAATCTAGGTGC
CAAGGAGGCGTTAAATACACGGAAAATTTTAGATGTATTTTTAAATTTAAAGGATTTTAAAAGTAGTAATCATATTATTCTAT
GCCATGAATTGTTATCTAATAATCCTCTTTTACTAAGTGAAAATAATGATTATTTGAGAAAAATAATTAATTGTAATCTAAGG
AGAATATCAATTAACTTTATATTGGATGAAATTTCATTTAATGAAAAGCTAACCAGATTCTGGTATAAACAGGCAGTACTAAA
TAATCTTACTGAAGCTATCCAATATTTCTATCAAAAATATAAGCAGTTTAAAGACTGGCGACTAATATGTGGACTTGCTTTAA
ATAATGTATTTGATCTTCATGAAATATATAACAAAGAGAAGGCTGATATAGATATTAATCAAATGATAGAGATAACCTGTACA
TATATGTGTAGTTATCCCACCATTTACTATTGTTTTGTAATGGGGGCTGACATTAATCGGGCAATGATTACCTCAGTTACAAA
ATCTTATACTGGTAACTTGTTCTTTTGTATAGATTTAGGAGCTACTGCCTTCGAAGAGTGCTTAGAAATAGCAAAACAACAGA
ATGATAATGAATTGGTAAAGGTATTATCATTGAAAAACTATTACAGTCCAGATAGCTCCCTTTTATCATTAAAAATAACAGAT
CCGGAAAAAATTAATATCTTATTAGATGATGAAACATATGAGTCAAAAAATGAGTTAATATATGAAGAATTAATATAA
Kenya 1950 MGF 360 14L
SEQ ID No. 131
ATGTCCACACCATCTTCTTTACAAGTCCTTGTCAAAAGGGTGCTGGACTTCCAGCATGTGTCTGAAGATGATTACTGTATTTT
AAAATGTTGTGGGTTATGGTGGCATGGAGGCCCAATCATGCTTTCTACGAATGAGGATAATCAAATGATGATAAAATCAGCAA
GTTTTAAAGATGGTTTAGAGATAAATCTCGCATTAATGATGGCCGTACAAGAAAACAATTGTAGCCTAATAGAGCTGTTTACC
GAATGGGGTGCAGACATTAATTCTGGATTGGTTACTGTCAACACGGAATACACCCGAAACCTATGTCGAAATCTAGGTGCTAA
GGAGACATTAAATAAACGGGAAATTTTAGATGTATTTTTAAAATTAAAGAATTTTAAAAGTAGTAATAATATTATTCTATCCC
ATGAATTATTATCTAATAATCCTCTTTTCCTAAGTGAAGATAATGATTATTTTAGAAGAATAATTAATTGTAATCTAAGGAGA
ATATCAATTAACTTTATATTGGATGAAATTTCATTTAATGAAAAGCTAACCAGATTCTGGTATAGTCAGGCGGTACTATATAA
TCTTACTGAAGCTATCCAATATTTCTATCAAAAATATGAACATCTCAATGAGTGGCGTTTAATTTGTGCGCTTTCTTTTAATA
ATGTGTTTGATCTTCATGAAATATATAACAAAGAAAAGGTTGGTATGGATATTAATCAAATGATAGAGATAACCTGTGCATAT
ATGTGTAGTTATTCCACCATTTACTATTGTTTTGTAATGGGAGCTGACATTAATCGGGCAATGATTATCTCGGTTACAAAATC
TTATACTTATAACTTGTTCTTTTGTATAGATTTAGGAGCTACTGCCTTCGAAGAGTGTTTAGAAATAGCAAAACAACAGAATA
ATAATGAATTAGTAAAAATATTATCATTGAAAAACTATTATAGTCCAGATAGCTCCCTTATATCATTAAAAATAACAGATCCG
GAGAAAATTAATATCTTATTAGATGAGGAAAATTATGAGTCAAAAAATGAGTTAATATATGAAGAATCTAATATAAATAATAG
CGATGATATATTTTGA
Ken06.Bus MGF 360 14L
SEQ ID No. 132
ATGCCATCTACTTTACAAGCACTTGCTAAAAAAGTACTGGCCACACCGCATATATCTAAAAAATATTGTCAGTCTAAGGAGTA
CTGTCATATATTAAAATGTTGTGGTTTATGGTGGCATGATGCTCCAATTACAATTTATCCTTGTATAAATAAAATATTGATAA
AAACAGCAAGCTTTAAACATGGTTTAGAGTTGAATGTCGCATTCATGAAAGCTGTACAGGAAAATAATTATGATCTAATAAGG
CTGTTTATTGAGTGGGGTGCGGATATCAACTTTGGGTTGGCCACTGTTAATACAGACCGTACCCGGGACCTGTGCCGGGAACT
AGGTGCGAAGGAAGCTTTAAGTGAAAAAGAAATTTTAGAAATATTTGATAAAATACAGTATATTAAAAGTAGCAGTAATATTA
TTGTATCCCATGAATTATTATCTAATAACCCCCTTTTCTTAAATAATGATCAATTGAAATTAAGAATGTTCGATGAACTACAT
AAACTATCAATCAACTTTGTATTACATGAGATATCATTTAATGAAATGCTAACTAGGTATTGGTATAGTATGGCGATACTATA
TAACCTCACCGCAGCCATTCAATATTTTTATCAATCCTACAAGCATTTTAAAGATTGGCGGTTAATATGTGGGCTTGCTTATA
ACAACGTGTTTGATCTTCATGAAATATATAACAAAGAGAAGGTTGATATGGATATTAATCAAATGATGCAGTTGGCCTGTATG
TATGATGGTAATTATACAACCATTTATTATTGTTTTATGTTGGGAGCTGACATTAATCGGGCAATGATTACCTCGGTTACAAA
ATCTTGTGATGGTAACTTATTCCTTTGTATAGATTTGGGGGCTGATGTATTTGAAGAGAGTATGAAAATGGCAATAGAAGATC
ATAATGACGTATTAGAAAGTATCTTATCATTTAAAAATTATTATAGTTCAGACGTTTCTCTGTTATCATTAAAGACGACAGAT
CCAGAAAAAATTAATGCCTTGTTAGAAGAAGAAAGTTACAAATCAAAAAATAGGTTGATATATAAAAGTTATTGA
Malawi Lil-20/1 (1983) MGF 360 14L
SEQ ID No. 133
ATGATGCCATCTACTTTACAAACACTTGCTAAAAAAACATTGGCCACACAGCATATATCTAAAAAATATTGGCCGTCTGAGGA
GTATTGTTATATATTAAAATGTTGTGGTTTATGGTGGCATGATTCTCCAATCACGATTTTTACTTGTATAAAACAAATATTAA
TAAAAACAGCAAACTTTAAACATGGTTTAGACTTAAACCTTGCATTAATGAAAGCTGTACAAGAAAATAACTATGAATTGATC
ATGTTGTTTACTGAGTGGGGTGCGGATATCAACTTGGGGTTGATCACTGTTAATACAGAGTGCACCCGAGATTTGTGCCAAAA
GTTAGGTGCAAAGGAAGCTTTGAGTGCAAAGGAAATTTTAGAAATATTTTATAAAATACAGTATATTAAGAGTAGCAATAATA
TTATTATATCTCATGAATTAATATCTAATCATCCCCTTTTCCTAAATAATGATCAATTGAAATTAAGAATTGTTGGTGAACTA
AATGCTATATCAATCAACTTTATATTGGATGAGATATCATTCAATGAAATGCTAACTAGGTATTGGTATAGTATGGCGATACT
ATATAAACTCCCTGCAGCCATTCAATATTTTTATCAATCATACAAGTATTTTAAAGATTGGCGGTTAATATGTAGCCTTGCTT
ATAACAACGTGTTTGACCTTCATGAAATTTATAATAAAGAGAAGACTGATATAAACATTGATGAAATGATGCGGTTGGCCTGT
AGGTATGATGGTAATTATACAACTATTTATTATTGTTTTATGTTGGGGGCTGACATTAATCAGGCAATGATTACCTCGGTCAT
GAATCTTTGGGACGGTAACTTATTTCTTTGTATAGATTTGGGGGCTGATGTATTTGAAGAGTGTATGAAAATAGCAATAGAAG
ATCATAATGGTGTATTAGAAAGTATCTTATCATTTAAAAATTATTATAGTCCAGACGTTTCTCTGTTATCATTAAAAACGACA
GATCCGGAAAAAATTAATGACTTGCTAGATGAAGATATCTATAAATCAAAAAATAGGTTGATATATAAAAGTTGTTGA

In an embodiment the attenuated ASFV of the invention comprises a functional version of 5 MGF 360 21R. Suitably the functional version of MGF 360 21R comprises the sequence of SEQ ID No. 124, 125, 126, 127, 128, 129, 130, 131, 132 or 133. Suitably the functional version of MGF 360 21R comprises a sequence having at least 70%, at least 80%, at least 90% or at least 95% identity with SEQ ID No. 124, 125, 126, 127, 128, 129, 130, 131, 132 or 133. Suitably the functional version of MGF 360 21R consists of the sequence of SEQ ID 10 No. 124, 125, 126, 127, 128, 129, 130, 131, 132 or 133.

Georgia 2007/1 MGF 505 1R (LR743116.1:28707-30302)
SEQ ID No. 134
ATGTTCTCTCTCCAGAACTTATGTCGAAAAACATTACCTAACCGTAAACTTCCTGAATTTTTTGACGAATATATATTACAACT
GCTGGGATTATACTGGGAAAACCATGGAACTATTCAACGAGCAGGAAACAACTGTGTGCTTATACAGCAACATACCCTCATTC
CCGTAAATGAAGCCCTGAGAACAGCAGCATCTGAAGAAAATTATGAGATCGTGAGCCTTTTATTAGCGTGGGAGGGGAACCTT
TACTATGCTATTATAGGGGCTCTAGAGGGCAACCGCCACGACTTAATTCGTAAATATGATGACCAAATCAAGGACCATCATGA
AATTCTGCCATTCATTGACGATCCAGTCATATTTCACAAATGCCATATCATGCGGCAATGCTTTTTTGATTGTATTTTATATC
AAGCTGTAAAATATAGTAAGTTTCGCGTTCTTCTTTACTTTAAACATAGATTAGAGGATGATTTGCCCTTCACTCATTTACTT
ATTGAAAAGGCATGTAAAGATCATAATTATGAAGTTATTAAATGGATATATGAAAACCTACATATCTACAATATGATAGATAC
CTTTGAATGTGCTATTGCCCATAAGGATCTACATCTATATTGTTTGGGGTATAGATTTATATATAACAGAATCGTACCCGATA
AGTATCATCATTTAGATATTCGCATGCTTTCAAGCCTACAACTCCTACATAAGGTGGCAGCCAAAGGATACTTAGATTTTATC
CTAGAAACCTTAAAGTATGATCATAATAAAGATAATATAAATATTATTCTAACACAAGCTGCAACCTATAACCATAGAAAAAT
TTTAATCTATTTCATTCCTCAATCAACCCACGCACAGATAGAACAATGTTTACTAGTGGCGATAAAAGCAAAATCTTCCAGGA
AAACCTTGAACTTACTACTGTCTCACCTAAACCTTTCCATCAACCTCATCAAAAAAATAAGCCATTATGTTGCCACTTACAAT
TCAACAAATATAATAGGCATTCTGAGTATGCGGCGGAAAAAGAAGATATATTTAGATATCATATTGACAAAATTTGTAAAAAA
AGCTATTTTTAATAAGTTTGTCGTTCGATGTATGGATACATTTTCTATAAACCCGGAAAGAATCCTTAAAATAGCCGCGCGAA
TAAATAGGATGATGTTAGTGAAAAAAATATCTGAACATGTTTGGAAAAATCATGCGGTTAGACTTAAATACCTTAAACATGCG
GTACACACGATGAAGCATAAAGATGGGAAAAATAGACTCATGAACTTTATCTATGATCGCTGTTATTACCATATGCAAGGGGA
AGAAATCTTTAGCCTCGCAAGATTTTATGCAATCCATCATGCACCAAAGTTGTTTGACGTTTTTTATGATTGTTGTATCCTAG
ATACGATACGATTCAAAAGCCTTCTTTTAGATTGTTCACATATCATAGGTAAAAACGCTCATGATGCTACCAATATCAACATC
GTGAACAAGTATATCGGCAACCTGTTTGTTATGGGAGTTCTTAGCAAAAAAGAAATCTTACAGGACTATCCATCCATTTATTC
TAAACAATACATGCCTTAG
China/2018/AnhuiXCGQ MGF 505 1R (MK128995.1:27736-29331)
SEQ ID No. 135
ATGTTCTCTCTCCAGAACTTATGTCGAAAAACATTACCTAACCGTAAACTTCCTGAATTTTTTGACGAATATATATTACAACT
GCTGGGATTATACTGGGAAAACCATGGAACTATTCAACGAGCAGGAAACAACTGTGTGCTTATACAGCAACATACCCTCATTC
CCGTAAATGAAGCCCTGAGAACAGCAGCATCTGAAGAAAATTATGAGATCGTGAGCCTTTTATTAGCGTGGGAGGGGAACCTT
TACTATGCTATTATAGGGGCTCTAGAGGGCAACCGCCACGACTTAATTCGTAAATATGATGACCAAATCAAGGACCATCATGA
AATTCTGCCATTCATTGACGATCCAGTCATATTTCACAAATGCCATATCATGCGGCAATGCTTTTTTGATTGTATTTTATATC
AAGCTGTAAAATATAGTAAGTTTCGCGTTCTTCTTTACTTTAAACATAGATTAGAGGATGATTTGCCCTTCACTCATTTACTT
ATTGAAAAGGCATGTAAAGATCATAATTATGAAGTTATTAAATGGATATATGAAAACCTACATATCTACAATATGATAGATAC
CTTTGAATGTGCTATTGCCCATAAGGATCTACATCTATATTGTTTGGGGTATAGATTTATATATAACAGAATCGTACCCGATA
AGTATCATCATTTAGATATTCGCATGCTTTCAAGCCTACAACTCCTACATAAGGTGGCAGCCAAAGGATACTTAGATTTTATC
CTAGAAACCTTAAAGTATGATCATAATAAAGATAATATAAATATTATTCTAACACAAGCTGCAACCTATAACCATAGAAAAAT
TTTAATCTATTTCATTCCTCAATCAACCCACGCACAGATAGAACAATGTTTACTAGTGGCGATAAAAGCAAAATCTTCCAGGA
AAACCTTGAACTTACTACTGTCTCACCTAAACCTTTCCATCAACCTCATCAAAAAAATAAGCCATTATGTTGCCACTTACAAT
TCAACAAATATAATAGGCATTCTGAGTATGCGGCGGAAAAAGAAGATATATTTAGATATCATATTGACAAAATTTGTAAAAAA
AGCTATTTTTAATAAGTTTGTCGTTCGATGTATGGATACATTTTCTATAAACCCGGAAAGAATCCTTAAAATAGCCGCGCGAA
TAAATAGGATGATGTTAGTGAAAAAAATATCTGAACATGTTTGGAAAAATCATGCGGTTAGACTTAAATACCTTAAACATGCG
GTACACACGATGAAGCATAAAGATGGGAAAAATAGACTCATGAACTTTATCTATGATCGCTGTTATTACCATATGCAAGGGGA
AGAAATCTTTAGCCTCGCAAGATTTTATGCAATCCATCATGCACCAAAGTTGTTTGACGTTTTTTATGATTGTTGTATCCTAG
ATACGATACGATTCAAAAGCCTTCTTTTAGATTGTTCACATATCATAGGTAAAAACGCTCATGATGCTACCAATATCAACATC
GTGAACAAGTATATCGGCAACCTGTTTGTTATGGGAGTTCTTAGCAAAAAAGAAATCTTACAGGACTATCCATCCATTTATTC
TAAACAATACATGCCTTAG
Tengani 62 MGF 505 1R (AY261364.1:22891-24486)
SEQ ID No. 136
ATGTTCTCTCTTCAGAACCTATGTCGAAAAACATTACCTGACTGTAAACTTCCTGAATTTTTTGACGAATATATATTACAACT
GCTGGGATTATACTGGGAAAACCATGGAACTATTCAACGAGCAGGAAACAACTGTGTGCTTATACAGCAACATAACCTCATTC
CCGTAAATGAAGCCCTAAGAATAGCAGCATCTGAAGAAAATTATGAGATCGTGAGTCTTTTATTAGCATGGGAGGGGAACCTT
TACTATGCTATTATAGGGGCTCTAGAGGGCAACCGCCACGACTTAATTCGTAAGTATGATGACCAAATCAAGGACCATCATGA
AATTCTGCCATTCATTGACGATCCAGTAATATTTCACAAATGCCATATCATGCGGCAATGCTTTTTTGATTGTATTTTATATC
AAGCTGTAAAATATAGTAAGTTTCGCGTTCTTCTTTCTTTTAAACATAGATTAAGGGATGATTTGCCCTTCACTCATTTACTT
ATTGAAAAGGCATGTAAAGATCATAATTATGAAGTAATTAAATGGATATATGAAAACCTACATATCTACAATATGATAGATAC
CTTTGAATGTGCTATTGCCCATAAGGATCTACGTCTATATTGTTTGGGGTATAGATTTATATATAACAGGATCGTACCCGATA
AGTATCATCATTTAGATATTCGCATGCTTTCAAGCCTACAACTCCTGCATAAGGTGGCAGCCAAAGGATACTTAGATTTTATC
CTAGAAACCTTAAAGTATGATCATAATAAAGATAATATAAATATTATTCTAACACAAGCTGCAACCTATAACCATAGAAAAAT
TTTAATCTATTTCATTCCTCAATCAACCCACGCACAGATAGAACAATGTTTACTAGTGGCGATAAAAACAAAATCTTCCAAGA
AAACCTTGAACTTACTACTGTCTCACCTAAACCTTTCCATCAATCTCATCAAAAAAATAAGCCATTATGTTGCCACTTACAAT
TCAACAAATATAATAGGCATTCTGAGTATGCGGCGGAAAAAGAAGATATATTTAGATATCATATTGACAAAATTTGTAAAAAA
AGCTATTTTTAATAAATTTGTCGTTCGATGTATGGATACATTTTCTATAAACCCGGAAAGAATCCTTAAAATAGCCGCGCGAA
TAAATAGGACGATGTTAGTGAAAAAAATATCTGAACATGTTTGGAAAAATCATGCGGTTAGACTTAAATACCTTAAACATGCG
GTACACACGATGAAGCATAAAGATGGGAAAAATAGACTCATGAACTTTATCTATGATCGCTGTTATTACCATATGCAAGGGGA
AGAAATCTTTAGCCTCGCAAGATTTTATGCAATCCATCATGCACCAAAGTTGTTTGACGTTTTTTATGATTGTTGTATCCTAG
ATACGATACGATTCAAAAGCCTTCTTTTAGATTGTTCACATATCATAGGTAAAAACGCTCATGATGCTACCAATATCAACATC
GTGAACAAGTATATCGGCAACCTGTTTGTTATGGGAGTTCTTAGCAAAAAAGAAATCTTACAGGACTATCCATCCATCTATTC
TAAACATTACATGCCTTAG
Warmbaths MGF 505 1R (AY261365.1:27651-29246)
SEQ ID No. 137
ATGTTCTCTCTCCAGAACCTATGTCGAAAAACTTTACCTGACTGTAAACTTCCTGAATTTTTTGACGAATATATATTACAACT
GCTGGGATTATACTGGGAAAACCATGGAACTATTCAACGAGCAGGAAACAACTGTGTGCTTATACAGCAACGTACCCTCATTC
CCGTAAATGAAGCCCTGAGAATAGCAGCATCTGAAGAAAATTATGAGATCGTGGGCCTTTTATTAGCGTGGGAGGGGAACCTT
TACTATGCTATTATAGGAGCTCTAGAGGGCAACCGCCACGACTTAATTCGTAAATATGATGACCAAATCAAGGACCATCATGA
AATTCTGCCATTCATTGACGATCCAGTCATATTTCACAAATGCCATATAATCCGACGATGCTTTTTTAATTGTATTTTATATC
AAGCTGTAAAATATAGTAAGTTTCGCGTTCTTCTTTACTTTAAACATAGATTAGAGGATGATTTGCCCCTCACTCATTTACTT
ATTGAAAAGGCATGTGAAGATCATAATTATGAAGTTATTAAATGGATATATGAAAACCTACATACCTACGATATAATGGATAC
CTTTGAATGTGCTATTGCCCATAAGGATCTACGTCTATATTGTTTGGGGTATACATTTATATATAACAGGATCGTACCCTATG
AGTATCATCATTTAGATATTCTCATTCTTTCAAGCCTACAACTCCTGCATAAGGTGGCAGCCAAAGGATACTTAGATTTTATC
CTAGAAACCTTAAAGTATGATCATAACAATGATAATTTAGATATTATTCTAACACAAGCTGCAACATATAACCATAGAAAAAT
TTTAACCTATTTCATTCCTCAATTAACCTACGCACAAATAGAACAATGTTTGTTCATGGCGATAAAAAGAAAATCTTCCAAGA
AAACCTTGAACTTACTACTGTCTCACCTAACCCTTTCCATCGAACTCATCAAAAAAATAAGCCAATATGTTGTCACTTACAAT
TCAACAAATATAATAGGCATTCTGAGTATGAAGCGGAAAAAGAAGATATATTTAGATATCATGTTGACAAAATATGTAAAATA
CGCTATTTTTAATAAATATGTCGTTCGATGTATGGATAGATTTTCCATAAACCCGGAAAGAATCATCAAAATGGCCGCGCGAA
TAAATAGGATGATGTTAGTGAAAAAAATATCTGAACATGTTTGGAAAAATCATGCGGCTAGACTTAAACACCTTAAGCATGCG
GTACACACGATGAAGCATAAAGATGGGAAAAATAGACTCATGAACTTTATCTATGATCGCTGCTATTACCATATGCAAGGGGA
AGAAATCTTTAGCCTCGCAAGATTTTATGCAATCCATCATGCACCAAAGTTGTTTGACGTTTTTTATGATTGTTGTATCCTAG
ATACTATACAATTCAAAAGCCTTCTTTTAGATTGTTCACATATCATAGGTAAAAACGCTCATGATGCTACCAATATCAACATC
GTGAACAAGTATATTGGCGACCTGTTTGCTATGGGAGTTCTTAGCAAAAAAGAAATCTTACAGGACTATCCATCCATCTATTC
TAAACATTACATGCTTTAG
Mkuzi 1979 MGF 505 1R (AY261362.1:29425-31020)
SEQ ID No. 138
ATGTTCTCTCTCCAGAACTTATGTCGAAAAACATTACCTGATTGTAAACTTCCTGAATTTTTTGACGATTATATATTACAACT
GCTGGGATTATACTGGGAAAACCATGGAACTATTCAACGGGCAGGAAACAACTGTGTGCTTATACAGCAACATACCCTTATTC
CCATAAATGAAGCCCTGAGAACAGCAGCATCTGAAGAAAATTATGAGATCGTGAGCCTTTTATTAGCATGGGAGGGGAATCTT
TACTATGCTATTATAGGGGCTCTAGAGGGCAACCGCCACGACTTAATTCGTAAATATGATGACCAAATCAAGGACCATCATGA
AATTCTGCCATTCATTGACGATCCAGTAATATTTCACAAATGCCATATCATGCGGCGATGCTTTTTTGATTGTATTTTATATC
AAGCTGTAAAATATAGTAAGTTTCGCGTTCTTCTTTATTTTAAATATAGGTTAGAGGATGATTTACCCCTCACTCATTTACTT
ATTGAAAAGGCATGTGAAAATCATAATTATGAAGTTATTAGATGGATATATGAAAACCTACATATCTACAATATGATAGATAC
CTTTGAATGTGCTATTGCCCATAAGGATCTACGTCTATATTGTTTGGGGTATACATTTATATATAACAGGATCGTACCCAATA
AGTATCATCATATAGATATTCTCATTCTTTCAAGCCTACAACTCCTGCATAAGGTGGCAGCCAAAGGATACTTAGACTTTATC
CTAGAAACCTTAAAGTATGATCATAACAATGATAATTTAGATATTATTCTAACACAAGCTGCAACATATAACCATAGAAAAAT
TTTAACCTATTTTATTCCTCAATCAACCTACGCACAAATAGAACAATGTTTGATGGTGGCGATAAAAACAAAATCTTCCAAGA
AAACCTTGAATTTACTATTGTCCCACCTAAACCTTTCCATCAAACTCATCAAAAAAATAAGCCAATATGTTGTCACTTACAAT
TCAACAAATATAATAGGCATTCTGAGTATGAAGCGAAAAAAGAAGATATATTTAGATATCTTGTTGACAAAATTTGTAAAAAA
TGCTATTTTTAATAAATTTGTCGTTCGATATATGGATACGTTTTCTATAAACCCGGAAAAAATCGTCAAAATGGCCGCGCGAA
TAAATAAGATGATGTTAGTGAAAAAAATATCTGAACATATTTGGAAAAATCATGCGGCTAGACTTGAACACCTTAAACATGCG
GTACACACGATGAAGCATAAAGATGGGAAAAATAGACTCATGAACTTTATCTATGAGTACTGCTATTACCATATGCAAGGGGA
AGAAATTTTTAGCCTCGCAAGATTTTATGCAATCCATCATGCACCAAAGTTGTTCGACGTTTTTTATAATTGTTGTATCCTAG
ATACGATACGATTCAAAAGCCTTCTTTTAGATTGTTCACATATCATAGGTAAAAACGCTCATGATGCTACTAATATCAACATC
GTGAACAAGTATATTGACAACCTGTTTGCTATGGGAGTTCTTAGCAAAAAAGAAATCTTACAGGACTATCCATCCATTTATTC
TAAACATTATATGCCTTAG
Warthog MGF 505 1R (AY261366.1:24387-25982)
SEQ ID No. 139
ATGTTTTCTCTCCAGAACTTGTGTCGAAAAACATTACCTGACTGTAAACTTCCTGAATTTTTTGACGAATATATATTACAACT
GCTGGGATTATACTGGGAAAACCATGGAACTATTCAACGAGCAGGAAACAACTGTGTGCTTATACAGCAACATACCCTCATTC
CCGTAAACGAAGCCCTGAGAATAGCAGCATCTGAAGAAAATTATGAGATCGTGAGCCTTTTATTAGCGTGGGAGGGAAACCTT
TACTATGCTATTATAGGAGCTCTAGAGGGCAACCGCCACGACTTAATTCGTAAATATGATGACCAAATCAAGGACCATCATGA
AATTCTGCCATTCATTGACGATCCAGTCATATTTCACAAATGCCATATCATGCGGCGATGCTTTTTTGATTGTATTTTATATC
AAGCTGTAAAATATAGTAAGTTTCGCGTTCTTCTTTATTTTAAATATAGGTTAGAGAATGATTTGCCCCTCGCTCATTTACTT
ATTAAAAAGGCATGTGAAGATCATAATTATGAAGTTATTAAATGGATATATGAAAACCTACATATCTACAATATAATGGATAC
CTTTGGATGTGCTATTGCCCATAAGGATCTACGTCTATATCGTTTGGGGTATACATTTATATATAACAGGATCGTACCCTATA
AGTATCATTATTTAGATGTTCTCATTCTTTCAGGCCTACATCTCCTGTATAAGGTGGCAGCCAAAGGATACTTAGATTTTATC
CTAGAAACCTTAAAGTATGATCATAACAATGATAATTTAGATATTATTCTAACACAAGCTGCAACATATAACCATAGAAAAAT
TTTAACCTATTACATTCCTCAATTAACCTATGCACAAATAGAACAATGTTTGTTCATGGCGATTAAAAAAAAATCTTCCAAGA
AAACCTTGAACTTACTACTGTCTCACCTAAAGCTTTCCATCAAACTCATCAAAAAAATAAGCCAATATGTTGCCACTTACAAT
TCAACAAATATAATAGGCATTCTGAATATGCGGCGGAAAAAGAAGATATATTTAGATATCATATTGACAAAATTTGTAAAAAA
AGCTATTTTTAATAAATTTGTCGTTCGATGTATGGATACATTTTCCATAAACCCGGAAAGAATCATCAAAATGGCCGCGCGAA
TAAATAAGATGTTGTTAGTGAAAAAAATATCTGAACATGCTTGGAAAAATCATGCGGCTAGACTTAAACACCTTAAGCATGCG
GTATACACGATGAAACATAAAGATGGGAAAAATAGACTCATGAACTTGATCTATGATCACTACTATTACCATATGCAAGGGGA
AGAAATCTTTAGCCTCGCAAGATTTTATGCAATCCATCATGCACCAAAGTTGTTTGACGTTTTTTATGATTGTTGTCTCCTAG
ATACTATACGATTTAAAAACCTTCTTTTAGATTGTTCACACATCATAGGTAAAAACGCTCATGATGCTACTAATATCACTATC
GTGAACAAGTATATTGGCAACCTGTTTGCTATGGGAGTTCTTAGCAAAAAAGAAATCTTACAGGACTATCCATCCATCTATTC
TAAACATTACATGCCTTAG
L60 MGF 505 1R (NC_044941.1:22320-23915)
SEQ ID No. 140
ATGTTCTCTCTCCAGAACTTATGTCGAAAAACATTACCTGACTGTAAACTTCCTGAATTTTTTGACGATTATATATTACAACT
GCTGGGATTATACTGGGAAAACCATGGAACTATTCAACGGGCAGGAAACAACTGTGTGCTTATACAACAACATACCCTCATTC
CCGTAAATGAAGCCCTAAGAATAGCAGCATCTGAAGAAAATTATGAGATCGTGGGCCTTTTATTAGCGTGGGAGGGGAACCTT
TACTATGCTATTATAGGGGCTCTAGAGGGTAACCGCTACAACTTAATTCGTAAATATGATGATCAAATCAAGGACCATCATGA
CATTCTGCCATTCATTGATGATCCAATCATATTTCACAAATGCCATATCATGCGGCGATGCTTTTTTGATTGTATTTTATATC
AAGCTGTAAAATATAGTAAGTTTCGTGTTCTTCTTTATTTTAAATATACATTAGAGGATGATTTGCCCCTCGTTCATTTACTT
ATTGAAAAGGCATGTGAAGATCATAATTATGAAGTTATTAAATGGATATATGAAAACCTACATGTCTGCCATATAATAGATAC
CTTTGACTGTGCTATTGCCCATAAAGATCTACGTTTATATTGTTTGGGGTATACATTTATATACAACAGGATTGTACCCTATA
AGTATCATCATTTAGATATTCTCATACTTTCAAGCCTACAACTCCTACATAAGGTGGCGGCCAAAGGATACTTAGATTTTATC
CTAGAAACCTTAAAGTATGATCATAATATAGATAATTTAGATGTTATTCTAACACAAGCTGCAACATATAACCATAGAAAAAT
TTTAACCTATTTTATTCCTCAATCAACCTACGCACAAATAGAACAATGTTTGTTCGTGGCGATAAAAACAAAATCTTCCAAGA
AAACCTTGAACTTACTACTGTCTCACCTAAACCTTTCCATCAAACTCATCCAAAAAATCAGCCAATATGTTGCCACTTTCAAT
TCAACAAATATAATAGGCATTCTGAGTATGAAGCGGAAAAAGAAGATATATTTGGATATCATATTGACAAAATTTGTAAAAAA
TGCTATTTTTAATAAATTTGTCGTTCGATGTATGGAGAGATTTTCTATAAACCCGGAAAGAATCGTCAAAATGGCTGCGCGTA
TAAATAAGATGATGTTAGTGAAAAAAATATCTGAACATGTTTGGAAAAATCATGCGGCTAGACTTAAACACCTTAAACATGCG
GTACACACGATGAAGCATAAAGATGGGAAAAATAGACTCATGAACTTTATCTATGAACACTGCTATTACCATATGCAAGGGGA
AGAAATTTTTAGCCTCGCAAGATTTTATGCAATCCATCATGCACCAAAGTTGTTCGACGTTTTTTATAATTGTTGTATCCTAG
ATACGATACGATTCAAAAGCCTTCTTTTAGATTGTTCACATATCATAGGTAAAAACGCTCATGATGCTACTAATATCAACATC
GTGAACAAGTATATTGGCAACCTGTTTGCTATGGGAGTTCTTAGCAAAAAAGAAATCTTACAGGACTATCCATCCATCTATTC
TAAACATTATATGCCTTAG
Benin 97/1 MGF 505 1R (NC_044956.1:21971-23566)
SEQ ID No. 141
ATGTTCTCTCTCCAGAACTTATGTCGAAAAACATTACCTGACTGTAAACTTCCTGAATTTTTTGACGATTATATATTACAACT
GCTGGGATTATACTGGGAAAACCATGGAACTATTCAACGGGCAGGAAACAACTGTGTGCTTATACAACAACATACCCTCATTC
CCGTAAATGAAGCCCTAAGAATAGCAGCATCTGAAGAAAATTATGAGATCGTGGGCCTTTTATTAGCGTGGGAGGGGAACCTT
TACTATGCTATTATAGGGGCTCTAGAGGGTAACCGCTACAACTTAATTCGTAAATATGATGATCAAATCAAGGACCATCATGA
CATTCTGCCATTCATTGATGATCCAATCATATTTCACAAATGCCATATCATGCGGCGATGCTTTTTTGATTGTATTTTATATC
AAGCTGTAAAATATAGTAAGTTTCGTGTTCTTCTTTATTTTAAATATACATTAGAGGATGATTTGCCCCTCGTTCATTTACTT
ATTGAAAAGGCATGTGAAGATCATAATTATGAAGTTATTAAATGGATATATGAAAACCTACATGTCTGCCATATAATAGATAC
CTTTGACTGTGCTATTGCCCATAAAGATCTACGTTTATATTGTTTGGGGTATACATTTATATACAACAGGATTGTACCCTATA
AGTATCATCATTTAGATATTCTCATACTTTCAAGCCTACAACTCCTACATAAGGTGGCGGCCAAAGGATACTTAGATTTTATC
CTAGAAACCTTAAAGTATGATCATAATATAGATAATTTAGATGTTATTCTAACACAAGCTGCAACATATAACCATAGAAAAAT
TTTAACCTATTTTATTCCTCAATCAACCTACGCACAAATAGAACAATGTTTGTTCGTGGCGATAAAAACAAAATCTTCCAAGA
AAACCTTGAACTTACTACTGTCTCACCTAAACCTTTCCATCAAACTCATCCAAAAAATCAGCCAATATGTTGCCACTTTCAAT
TCAACAAATATAATAGGCATTCTGAGTATGAAGCGGAAAAAGAAGATATATTTGGATATCATATTGACAAAATTTGTAAAAAA
TGCTATTTTTAATAAATTTGTCGTTCGATGTATGGAGAGATTTTCTATAAACCCGGAAAGAATCGTCAAAATGGCTGCGCGTA
TAAATAAGATGATGTTAGTGAAAAAAATATCTGAACATGTTTGGAAAAATCATGCGGCTAGACTTAAACACCTTAAACATGCG
GTACACACGATGAAGCATAAAGATGGGAAAAATAGACTCATGAACTTTATCTATGAACACTGCTATTACCATATGCAAGGGGA
AGAAATTTTTAGCCTCGCAAGATTTTATGCAATCCATCATGCACCAAAGTTGTTCGACGTTTTTTATAATTGTTGTATCCTAG
ATACGATACGATTCAAAAGCCTTCTTTTAGATTGTTCACATATCATAGGTAAAAACGCTCATGATGCTACTAATATCAACATC
GTGAACAAGTATATTGGCAACCTGTTTGCTATGGGAGTTCTTAGCAAAAAAGAAATCTTACAGGACTATCCATCCATCTATTC
TAAACATTATATGCCTTAG
Pretorisuskop/96/4 MGF 505 1R (AY261363.1:28449-30044)
SEQ ID No. 142
ATGTTCTCTCTCCAGAACTTATGTCGAAAAACATTACCTGACTGTAAACTTCCTGAATTTTTTGACGAATATATATTACAACT
GCTGGGATTATACTGGGAAAACCATGGAACTATTCAACGAGCAGGAAACAACTGTGTGCTTGTACAACAACATACCCTCATTC
CCGTAAACGAAGCCCTGAGAATAGCAGCGTCTGAAGAAAATTATGAGATCGTGAGCCTTTTATTAGCGTGGGAGGGAAACCTT
TACTATGCTATTATAGGAGCTCTAGAGGGCAACCGCCACGACTTAATTCGTAAATATGATGACCAAATCAAGGACCATCATGA
AATTCTGCCATTCATTGACAATCCAGTCATATTTCACAAATGCCATATAATGCGGCGATGCTTTTTTGATTGTATTTTATATC
AAGCTGTAAAATATAGTAAGTTTCGCGTTCTTCTTTATTTTAAATATAGGTTAGAGAATGATTTGCCCCTCGCTCATTTACTT
GTTGAAAAGGCATGTGAAGATCATAATTATGAAGTTATTAAATGGTTATATGAAAACCTACATATCTACAATATAATGGAGAC
CTTTGAATGTGCTATTGCCCATAAGGATCTACGTCTATATCGTTTGGGGTATACATTTATATATAACAGGATCGTACCCTATA
AGTATCATTATTTAGATGTTCTCATTCTTTCAGGCCTACATCTCCTGTATAAGGTGGCAGCCAAAGGATACTTAGATTTTATC
CTAGAAACCTTAAAGTATGATCATAACAATGATAATTTAGATATTATTCTAACACAAGCTGTAACATATAACCATAGAAAAAT
TTTAACCTATTACATTCCTCAATTAACCTATGCACAAATAGAACAATGTTTGTTCATGGCGATTAAAAAAAAATCTTCCAAGA
AAACCTTGAACTTACTACTGTCTCACCTAAAGCTTTCCATCAAACTCATCAAAAAAATAAGCCAATATGTTGCCACTTACAAT
TCAACAAATATAATAGGCATTCTGAATATGAAGCGGAAAAAGAAGATATATTTAGATATCATATTGACAAAATTTGTAAAATA
CGCTATTTTTAATAAATATGTCGTTCGTTGTATGGATACATTTTCCATAAACCCGGAAAGAATCATCAAAATGGCCGCGCGAA
TAAATAAGATGTTGTTAGTGAAAAAAATATCTCAACATGCTTGGAAAAATCATGCGGCTAGACTTAAACACCTTAAGCATGCG
GTATACACGATGAAACATAAAGATGGGAAAAATAGACTCATGAACTTGATCTATGATCACTACTATTACCATATGCAAGGGGA
AGAAATCTTTAGCCTCGCAAGATTTTATGCAATCCATCATGCACCAAAGTTGTTTGACGTTTTTTATGATTGTTGTCTCCTAG
ATACTATACGATTTAAAAGCCTTCTTTTAGATTGTTCACACATCATAGGTAAAAACGCTCATGATGCTACTAATATCACTATC
GTGAACAAGTATATTGGCAACCTGTTTGCTATGGGAGTTCTTAGCAAAAAAGAAATCTTACAGGACTATCCATCCATCTATTC
TAAACATTACATGCCTTAG
Kenya 1950 MGF 505 1R (AY261360.1:31904-33496)
SEQ ID No. 143
ATGTTTTCTCTCCAGAACTTATGTCGAAAAACCTTACCTGACCGTAAACTTCCTGAATTTTTTGACGATTATGTATTACAACT
GCTGGGATTATACTGGGAAAACCATGGAACGATTCAACGAGCAGGAAACAACTGTGTACTTATCCAACAACATAACCTCATTC
CCGTAAATGAAGCCCTAAGAATAGCAGCATCTGAAGAAAATTATGAGATCGTGAGCCTTTTATTAGCTTGGGAGGGGAACCTT
TACTATGCTATTATAGGGGCTCTAGAGGGTAATCGCCACAACTTAATTCGTAAATATGATGACCAAATCAAGGACCATCATGA
AATTCTGCCATTTATTGACGATCCAGTCATATTTCACAAATGTCATATGATGCGGCGATGCTTTTTTAATTGTATTTTGTATC
AAGCTGTAAAATATAGTAAGTTTAGCGTTCTTCTATATTTTAAATATATATTAAAGGAGAATTTGCCCCTCGTTCATTCACTT
ATTGAAAAGGCGTGTGAAGATCATAATTATGAAGTTATTAAATGGATCTATGAAAACCTACATATCTATGAAATAATGGATAC
CTTTAAATGTGCTATTGCCCATAAAGATCTACATTTATATAGTTTAGGATATACATTTATATATAATAGGATCGTACCCTATA
AGTATCATCATTTAGATATTCGCATACTTTCAAGGCTACAACTTCTACATAAGGTGACAGCCAAAGGATACTTGGACTTTATC
CTAGAAACCTTAAAGTATGATCATAATAAAGATAATATAAATATTATTCTGACACAAGCTGCAACCTATAACCATAGAAACAT
TTTAACCTATTTCATTCCTCAATCAACCTACGCGCAGATAGAACAATGCTTATTCGTGGCAATAAAAACAAACGCTTCCAAGA
AAACCTTGAATTTACTATTATCTCACCTAAACCTTTCTATCAAACTCGTCAAAAAATTAAGCCAATATGTTGTTGCTTACAAG
TCAACAAATATCATAAGCATTCTGAGTATGCAGCAAAAAAAGAAGATATATTTAGATATCATTTTGACAAAGGTTGTAAAAAA
CGCTATTTTTATTAAATTTGTCATTGGATGTATGGTTACATTTTCCATAAACCCAGAAAGAATTGTCAAAATGGCCGCGCGAA
TAAAAAAGATGAAGTTAGTGAAAAATATATCTGAACATGTTTGGAAAAATCATGCGGCTAAACTTAAACACCTTAAGCATGCG
GTACACACGATGAAGCATCAAGAAGGAAAAAATAGATTAATGAACTTTATCTATGATCACTGTTATTACCACATGCAAGGAGA
AGAGATCTTTAGTCTTGCAAGATTTTATGCAATCCATCATGCGCCAAAATTGTTCGACGTTTTTTATGATTGTTGTATCCTAG
ATACGATACGATTTAAAAGCCTGCTTTTAGATTGTTCATATATCATAGCTAAAAACGCTCATGATGCTAGTATTAACATCGTG
AATAAGTATATTGGCAATCTATTTGCTATGGGGGTTCTTAGCAAAAAAGAAATCTTACAAGACTATCCATCCATCTATTCTAA
GGATTACATGCTTTAG
Ken06.Bus MGF 505 1R (NC_044946.1:25784-27376)
SEQ ID No. 144
ATGTTCTCTCTCCAGAACTTATGTCGAAAAACCTTACCTGACTGTAAACTTCCTGAGTTTTTTGACGATTATGTATTACAACT
GCTGGGCTTATACTGGGAAAATCATGGTACGATTCAACGAGCAGGAAACAACTGTGTACTTATCCAACAACATAACCTCATTC
CCGTAAATGAAGCCCTAAGAATAGCAGCATCTGAGGAAAATTATGAGATCGTGAGCCTTTTATTAGCGTGGGAGGGGAACCTT
TACTATGCTATTATAGGGGCTCTAGAGGGGAACCACCACAACTTAATTCGTAAATACGATGACCAAATCAAGGACCATCATGA
AATTCTGCCATTTATTGACGATCCAGTCATATTTCACAAATGTCATACAATGCGGCGATGCTTTTTTAATTGTATTTTATATC
AAGCTGTAAAATATAGTAAGTTCAGCGTTCTTCTATACTTTAAATATATATTAAAGGAGAATTTGCCCCTCGTCCACTCACTT
ATTGAAAAGGCGTATAAATATCATAATTATGAGGTTATTAAATGGATCTATGAAAACCTACATATCTATGATATCATAAATAC
TTTTAAATGTGCTATTGCCCATAAAGATCTACGTTTATATTGTTTAGGGTATACATTTGTATATAATAGAATCGTACCCTATA
AGTATTATCATTTAGATATTCGCATCCTTTTAAGGCTACAACTTCTACATAAGGTGACAGCCAAAGGATACTTGGATTTTATC
CTAGAAACCTTAAAGTATGATCATAATACAAACAATATAGATATTATCCTAACACAGGCTGCAACCTATAACCATAGAAACAT
TTTAACCTATTTCATTCCTCAATCAACCTACGCACAAATAGAACAATGTTTATTCGTGGCGATAAAAACAAATGCTTCCAAGA
AAACCTTGAATTTACTATTATCTCACCTAAATCTTTCTATCAAACTCGTCAAAAAATTAAGCCAATATGTTGTCGCTTACAAG
TCAACAAATATCATAAGCATTCTGAGTAGGCAGCAAAAAAAGAAGATATATTTAGATATCATTTTGACAAAGGTTGTAAAAAA
TGCTGTTTTTAATAAATTTGTCATTGGATGTATGGTTACATTTTCCATAAACCCAGAAAGAATTGTCAAAATGGCCGCGCGAA
TAAAAAAGATGAAGTTAGTAAAAAATATATCTGAACATGTTTGGAAAAATCATGCGGTTAAACTTAAATATCTTAAGCATGCG
GTACACACGATGAAGCATCAAGAAGGAAAAAATAGATTAATGAACTTTATCTATGATCACTGTTATTACCACATGCAAGAAGA
AGAGATCTTTAGCCTCGCAAGATTTTATGCAATCCATCATGCGCCAAAATTGTTCGACGTTTTTTATGATTGTTGTATCCTAG
ATACGATACGATTTAAAAGCCTGCTTTTAGATTGTTCACATATCATAGTTAAAAACGCTCATGATGCTAGTATTAACATCGTG
AACAAGTATATTGGCAATCTATTTGCTATGGGAGTTCTTAGCAAAAAAGAAATCTTACAAGACTATCCATCTATCTATTCTAA
GGATTACATGCTTTAG
Ken05/Tk1 MGF 505 1R (NC_044945.1:29657-31249)
SEQ ID No. 145
ATGTTCTCTCTCCAAAACTTATGTCGAAAAACCTTACCTGACTGTAAACTTCCTGAATTTTTTGACGATTATGTATTACAACT
GCTGGGATTATACTGGGAAAACCATGGAACGATTCAACGAGCAGGAAACAACTGTGTACTTATCCAACAACATAACCTCATTC
CCGTAAATGAAGCCCTAAGAATAGCAGCATCTGAAGAAAATTATGAGATCGTGAGCCTTTTATTAGCGTGGGAGGGGAACCTT
TACTATGCTATTATAGGGGCTCTAGAGGGTAACCGCCACAACTTAATTCGTAAATATGATGACCAAATCAAGGACCATCATGA
AATTCTGCCATTTATTGACGATCCAGTTATATTTCACAAATGTCATATAATGCGGCGATGCTTTTTTAATTGTATGTTGTATC
AAGCTGTAAAATATAGTAAGTTTAGCGTTCTTCTATATTTTAAATATATATTAAAGGAAAATTTGCCCCTCGTCCACTCACTC
ATTGAAAAAGCGTATAAATATCATAATTATGAGGTTATTAAATGGATCTATGAAAATCTACATATCTATGATATCATAAATAC
TTTTAAATATGCTATTGCCCATAAAGATCTACGTTTATATTGTTTAGGGTATACATTTGTATATAATAGGATCGTACCCTATA
AGTATTATCATTTAGATATTCGCATCCTTTTAAGGCTACAACTTCTACATAAGGTGACAGCCAAAGGATACTTGGATTTTATC
CTAGAAACCTTAAAGTATGATCATAATACAAACAATATAGATATTATCCTAACACAGGCTGCAACCTATAACCATAGAAACAT
TTTAACCTATTTCATTCCTCAATCAACCTACGCACAAATAGAACAATGCTTATTCGTGGCAATAAAAACAAACGCTTCCAAAA
AAACCTTGAATTTACTATTATCTCACCTAAACCTTTCTATCAAACTCGTCAAAAAATTAAGCCAATATGTTGTCGCTTACAAG
TCAACAAATATCATAAGCATTCTGAGTATGCAGCAAAAAAAGAAGATATATTTAGATATCATTTTGACAAAGGTTGTAAAAAA
CGCTGTTTTTATTAAATTTGTCATTGGATGTATGGTTACATTTTCCATAAACCCAGAAAGAATTGTCAAAATGGCCGCGCGAA
TAAAAAAGATGAAGTTAGTAAAAAATATATCTGAACATGTTTGGAAAAATCATGCGGCTAAACTTAAACATCTTAAGCATGCG
GTACACACGATGAAGCATCAAGAAGGAAAAAATAGACTCATGAACTTTATCTATGATCACTGCTATTACCACATGCAAGGAGA
AGAGATCTTTAGTCTTGCAAGATTTTATGCAATCCATCATGCGCCAAAATTGTTCGACGTTTTTTATGATTGTTGTATCCTAG
ATACGATACGATTTAAAAGCCTGCTTTTAGATTGTTCACATATTATAGCTAAAAACGCTCATGATGCTAGTATTAACATCGTG
AATAAGTATATTGGCAATCTATTTGCTATGGGAGTTCTTAGCAAAAAAGAAATCTTACAAGACTATCCATCCATCTATTCTAA
ATATGACATACTTTAG
Malawi Lil-20/1 (1983) MGF 505 1R (AY261361.1:26041-27633)
SEQ ID No. 146
ATGTTCTCTCTCCAGAACTTATGTCGAAAAACATTACCTGACTGTAAACTTCCTGAATTTTTTGACGAATATATATTACAACT
GCTGGGATTATACTGGGAAAACCATGGAACTATTCAACGAGCAGGAAACAACTGTGTGCTTATCCAGCAACATAACCTTATTC
CCGTAAATGAAGCCCTAAGAATAGCAGCATCTGAAGAAAATTATGAGATCGTGAGCCTTTTATTAGCGTGGGAGGGAAACCTT
TACTATGCTATTATAGGGGCTCTAGAGGGCAACCGCCCCGACTTAATTCGTAAATATGATGACCAAATCAAGGACCATCATGA
AATTTTGCCATTCATCGACGATCCAATCATATTTCACAAATGTCACATAATGCGGCGATGCTTTTTTAATTGTATTTTATATC
AAGCTGTAAAATATAGTAAGTTTCGTGTTCTTCTTTATTTTAAACATAGGTTAGGGGATGATTTGCCCCTCACTCATTTACTT
ATTGAAAAGGCATGTGAAGATCATAATTATGAAGTTATTAAATGGATCTATGAAAACCTACATAGCTACAATATAATGGATAC
CTTTGAATGTGCTATTGCCCATAAGGATCTACGTCTATATTGTTTGGGGTATACATTTATATATAATAGGATCGTACCCTATA
AGTATCATCATTTAGATATTTGCATACTTTCAAGCCTACAACTCCTGCATAAGGTGGCAGCCAAAGGATACTTGGATTTTATC
CTAGAAACCTTAAAGTATGATCATAACATAAATAATATAGATATTATTCTAACACAAGCTGCAACCTATAACCATAGAAAAAT
TTTAACCTATTTCATTCCTCAATTAACCTACGCACAGATAGAACAATGTTTACTAGTGGCGATAAAAACAAAAGCTTCCAAGA
AAACCTTGAACTTACTACTGTCTCACCTAAACCTTTCCATCAAACTCATCAAAAAAATAAGCCAATATGTTGTCACTTACAAT
TCAACAAATATCATAAGCATTCTGAGTATGCGGCGGAAAAAGAAGATATATTTAGATATCATTTTGACAGAGTTTGTAAAAAA
CGCTATTTTTAATAAATTTGTCGTTCGATGTATGGATACATTTTCCATAAACCCGGAAAGAATTGTCAAAATGGCCGCACGAA
TAAATAGGATGATGTTAGTGAAAAATATATCTGAACGTGTTTGGAAAAATCATGCGGTTAAACTTAAACACCTTAAGCATGCG
GTACATACGATGAAGCATCAAGAAGGAAAAAATAGACTCATGAACTTTATCTATGATCACTGCTATTACCACATGCAAGGGGA
AGAGATCTTTGGCCTCGCAAGATTTTATGCAATCCATCATGCACCCAAGTTGTTTGACGTTTTTTATGATTGCTGCATGCTAG
ATGCTACACGATTTAAAAGCCTGCTTTTAGATTGTCCACATATCATAGGTAAAAACGCTTATGATGCTGGTATCAACCTCGTG
AACAAATATATTGGCAACCTATTTGCTATGGGGGTTCTTAGCAAAAAAGAAATCTTACAGGACTATCCATCTATCTATTCTAA
ACATGATATGTTTTAG

In an embodiment the attenuated ASFV of the invention comprises a functional version of MGF 505 1R. Suitably the functional version of MGF 505 1R comprises the sequence of 60 SEQ ID No. 134, 135, 136, 137, 138, 139, 140, 141, 142, 143, 144, 145 or 146. Suitably the functional version of MGF 505 1R comprises a sequence having at least 70%, at least 80%, at least 90% or at least 95% identity with SEQ ID No. 134, 135, 136, 137, 138, 139, 140, 141, 142, 143, 144, 145 or 146. Suitably the functional version of MGF 505 1R consists of the sequence of SEQ ID No. 134, 135, 136, 137, 138, 139, 140, 141, 142, 143, 144, 145 or 146.

Georgia 2007/1 MGF 5052R (NC_044959.1:33119-34699)
SEQ ID No. 147
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCATAGGAGACGACCACATACTCATACGACGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCTTGCAGGGTGATCAATATGACCTGATCCATAAGTATGAAAACCAAATCGGCGACTTTCATTTTATCTT
ACCATTGATTCAAGACGCGAATACGTTTGAAAAATGCCACGCTTTAGAACGTTTTTGTGGTGTTTCATGTCTGCTAAAACATG
CTACAAAATACAACATGCTCCCTATTCTCCAAAAATACCAAGAAGAGCTGTCTATGAGAGCGTATCTTCACGAAACCCTATTT
GAACTAGCATGCCTATGGCAGAGGTATGATGTCCTTAAATGGATAGAGCAAACCATACATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAACATCTCAAGAAGACAGCGGCCAAAGGGCTCCTCCACTTTGTGCTAGAAACGTTAAAATACGGCGGCAAC
ATAGATACCGTCCTGACCCAAGCCGTAAAGTACAATCATAGAAAACTTTTAGATTATTTTCTGCGTCAACTACCTCGTAAACA
TATTGAAAAACTTTTGTTGCTGGCCGTGCAGGAAAAGGCTTCTAAAAAAACATTGAACTTACTGTTGTCACATTTAAACTACT
CCGTGAAACGCATCAAAAAACTACCGCGCTATGTGATAGAGTACGAGTCCACCTTGGTGATAAAGATTTTATTAAAAAAAAGA
GTGAACCTGATAGATGCCATGTTGGAAAAGATGGTAAGATATTTTTCTGCGACGAAAGTGAGGACGATCATGGATGAGCTTTC
GATTAGTCCGGAAAGAGTCATTAAGATGGCTATACAGAAAATGAGAACGGATATCGTAATCCATACTTCTTATGTTTGGGAGG
ATGATCTAGAACGTCTTACTCGTCTTAAAAATATGGTATACACCATAAAGTACGAACATGGGAAAAAAATGTTAATTAAAGTC
ATGCACGGCATATACAAAAACTTATTATACGGCGAAAGGGAAAAAGTCATGTTTTATTTAGCCAAGCTCTATGTTGCTCAAAA
CGCGGCCACCCAATTCAGAGACATTTGTAAGGACTGTTACAAACTGGATGTGGCACGGTTTAAACCGCGGTTTAAGCAACTAA
TATTAGACTGTTTAGAAATTATTACTAAAAAATCTTGCTATAGTATCCTGGAAATCTTAGAAAAACATATTATTTCCCTGTTT
ACTATGAAAGTTATGACTGAAGAAGAAAAAAACCTATGTTTAGAAATATTATATAAAGTAATTCATTATAAAACAATACAATG
TTAA
China/2018/AnhuiXCGQ MGF 505 2R
SEQ ID No. 148
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCATAGGAGACGACCACATACTCATACGACGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCTTGCAGGGTGATCAATATGACCTGATCCATAAGTATGAAAACCAAATCGGCGACTTTCATTTTATCTT
ACCATTGATTCAAGACGCGAATACGTTTGAAAAATGCCACGCTTTAGAACGTTTTTGTGGTGTTTCATGTCTGCTAAAACATG
CTACAAAATACAACATGCTCCCTATTCTCCAAAAATACCAAGAAGAGCTGTCTATGAGAGCGTATCTTCACGAAACCCTATTT
GAACTAGCATGCCTATGGCAGAGGTATGATGTCCTTAAATGGATAGAGCAAACCATACATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAACATCTCAAGAAGACAGCGGCCAAAGGGCTCCTCCACTTTGTGCTAGAAACGTTAAAATACGGCGGCAAC
ATAGATACCGTCCTGACCCAAGCCGTAAAGTACAATCATAGAAAACTTTTAGATTATTTTCTGCGTCAACTACCTCGTAAACA
TATTGAAAAACTTTTGTTGCTGGCCGTGCAGGAAAAGGCTTCTAAAAAAACATTGAACTTACTGTTGTCACATTTAAACTACT
CCGTGAAACGCATCAAAAAACTACCGCGCTATGTGATAGAGTACGAGTCCACCTTGGTGATAAAGATTTTATTAAAAAAAAGA
GTGAACCTGATAGATGCCATGTTGGAAAAGATGGTAAGATATTTTTCTGCGACGAAAGTGAGGACGATCATGGATGAGCTTTC
GATTAGTCCGGAAAGAGTCATTAAGATGGCTATACAGAAAATGAGAACGGATATCGTAATCCATACTTCTTATGTTTGGGAGG
ATGATCTAGAACGTCTTACTCGTCTTAAAAATATGGTATACACCATAAAGTACGAACATGGGAAAAAAATGTTAATTAAAGTC
ATGCACGGCATATACAAAAACTTATTATACGGCGAAAGGGAAAAAGTCATGTTTTATTTAGCCAAGCTCTATGTTGCTCAAAA
CGCGGCCACCCAATTCAGAGACATTTGTAAGGACTGTTACAAACTGGATGTGGCACGGTTTAAACCGCGGTTTAAGCAACTAA
TATTAGACTGTTTAGAAATTATTACTAAAAAATCTTGCTATAGTATCCTGGAAATCTTAGAAAAACATATTATTTCCCTGTTT
ACTATGAAAGTTATGACTGAAGAAGAAAAAAACCTATGTTTAGAAATATTATATAAAGTAATTCATTATAAAACAATACAATG
TTAA
Mkuzi 1979 MGF 505 2R (AY261362.1:34826-36406)
SEQ ID No. 149
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCATAGGAGACGACCACATACTCATACGACGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCTTGCAGGGTGATCAATATGACCTGATCCATAAGTATGAAAACCAAATCGGCGACTTTCATTTTATCTT
ACCATTGATTCAAGACGCGAATACGTTTGAAAAATGCCACGCTTTAGAACGTTTTTGTGGTGTTTCATGTCTGCTAAAACATG
CTACAAAATACAACATGCTCCCTATTCTCCAAAAATACCAAGAAGAGCTGTCTATGAGAGCGTATCTTCACGAAACCCTATTT
GAACTAGCATGCCTATGGCAGAGGTATGATGTCCTTAAATGGATAGAGCAGACCATGCATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAACATCTCGAGAAGACAGCGGCCAAAGGGCTCCTCCACTTTGTGCTAGAAACGTTAAAATACGGCGGCAAC
ATAGATACCGTCCTGACCCAAGCCGTAAAGTACAATCATAGAAAACTTTTAGATTATTTTCTGCGTCAACTACCTCGTAAACA
TATTGAAAAACTTTTGTTGCTGGCTGTTCAGGAAAAGGCTTCTAAAAAAACATTGAACTTACTGTTGTCACATTTAAACTACT
CCGTGAAACGCATCAAAAAACTGCTGCGCTATGTGATAGAGTATGAGTCCACCTTGGTGATAAAGATTTTGTTAAAAAAAAGA
GTAAACCTGATAGATGCCATGTTGGAAAAGATGGTAAGATATTTTTCTGCGACAAAAGTGAGGACGATCATGGATGAGCTTTC
GATTAGTCCGGAAAGAGTCATTAAGATGGCTATACAGAAAATGAGAACGGATATCGTAATCCATACTTCTTATGTTTGGGAGG
ATGATCTAGAACGTTTTACTCGTCTTAAAAATATGGTATACACCATAAAGTACGAACATGGGAAAAAAATGTTAATTAAAGTC
ATGCACGGCATATACAAAAACTTATTATACGGCGAAAGGGAAAAAGTCATGTTTCATTTAGCCAAGCTCTATGTTGCTCAAAA
TGCGGCCACCCAATTCAGAGACATTTGTAAGGACTGTTACAAACTGGATGTGGCACGGTTTAAACCGCGGTTTAAGCAACTAA
TATTAGACTGTTTAGAAATTGTTACTAAAAAATCTTGCTATAGTATCCTGGAAATCTTAGAAAAACATATTATTTCCCTATTT
ACTATGAAAGTTATGACTGAAGAAGAAAAAAACCTATGTTTAGAAATATTATATAAAGTAATTCATTATAAAACAATACAATG
TTAA
L60 MGF 505 2R (NC_044941.1 27701-29281)
SEQ ID No. 150
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCATAGGAGACGACCACATACTCATACGACGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCCTGCAGGGTGATCAATATGACCTGATCCATAAGTATGAAAACCAAATCGGCGACTTTCATTTTATCTT
ACCATTGATTCAAGACGCGAATACGTTTGAAAAATGCCACGCTTTAGAACGTTTTTGTGGTGTTTCATGTCTGCTAAAACATG
CTACAAAATACAACATGCTCCCTATTCTCCAAAAATACCAAGAAGAGCTGTCTATGAGAGCGTATCTTCACGAAACCCTATTT
GAACTAGCATGCCTATGGCAGAGGTATGATGTCCTTAAATGGATAGAGCAGACCATGCATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAACATCTCGAGAAGACAGCGGCCAAAGGGCTCCTCCACTTTGTGCTAGAAACGTTAAAATACGGCGGCAAC
ATAGATACCGTCCTGACCCAAGCCGTAAAGTACAACCATAGAAAACTTTTAGATTATTTTCTGCGTCAACTACCTCGTAAACA
TATTGAAAAGCTTTTGTTGCTGGCTGTTCAGGAAAAGGCTTCTAAAAAAACATTGAACTTACTGTTGTCACATTTAAACTACT
CCGTGAAACGCATCAAAAAACTGCTGCGCTATGTGATAGAGTATGAGTCCACCTTGGTGATAAAGATTTTGTTAAAAAAAAGA
GTAAACCTGATAGATGCCATGTTGGAAAAGATGGTAAGATATTTTTCTGCGACGAAAGTGAGGACGATCATGGATGAGCTTTC
GATTAGTCCGGAAAGAGTCATTAAGATGGCTATACAGAAAATGAGAACGGATATCGTAATCCATACTTCTTATGTTTGGGAGG
ATGATCTAGAACGTCTTACTCGTCTTAAAAATATGGTATACACCATAAAGTACGAACATGGGAAAAAAATGTTAATTAAAGTC
ATGCACGGCATATACAAAAACTTATTATACGGCGAAAGGGAAAAAGTCATGTTTCATTTAGCCAAGCTCTATGTTGCTCAAAA
TGCGGCCACCCAATTCAGAGACATTTGTAAGGACTGTTACAAACTGGATGTGGCACGGTTTAAACCGCGGTTTAAGCAACTAA
TATTAGACTGTTTAGAAATTGTTACTAAAAAATCTTGCTATAGTATCCTGGAAATCTTAGAAAAACATATTATTTCCCTATTT
ACTATGAAAGTTATGACTGAAGAAGAAAAAAACCTATGTTTAGAAATATTATATAAAGTAATTCATTATAAAACAATACAATG
TTAA
Benin 97/1 MGF 505 2R (NC_044956.1:27352-28932)
SEQ ID No. 151
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCATAGGAGACGACCACATACTCATACGACGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCCTGCAGGGTGATCAATATGACCTGATCCATAAGTATGAAAACCAAATCGGCGACTTTCATTTTATCTT
ACCATTGATTCAAGACGCGAATACGTTTGAAAAATGCCACGCTTTAGAACGTTTTTGTGGTGTTTCATGTCTGCTAAAACATG
CTACAAAATACAACATGCTCCCTATTCTCCAAAAATACCAAGAAGAGCTGTCTATGAGAGCGTATCTTCACGAAACCCTATTT
GAACTAGCATGCCTATGGCAGAGGTATGATGTCCTTAAATGGATAGAGCAGACCATGCATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAACATCTCGAGAAGACAGCGGCCAAAGGGCTCCTCCACTTTGTGCTAGAAACGTTAAAATACGGCGGCAAC
ATAGATACCGTCCTGACCCAAGCCGTAAAGTACAACCATAGAAAACTTTTAGATTATTTTCTGCGTCAACTACCTCGTAAACA
TATTGAAAAGCTTTTGTTGCTGGCTGTTCAGGAAAAGGCTTCTAAAAAAACATTGAACTTACTGTTGTCACATTTAAACTACT
CCGTGAAACGCATCAAAAAACTGCTGCGCTATGTGATAGAGTATGAGTCCACCTTGGTGATAAAGATTTTGTTAAAAAAAAGA
GTAAACCTGATAGATGCCATGTTGGAAAAGATGGTAAGATATTTTTCTGCGACGAAAGTGAGGACGATCATGGATGAGCTTTC
GATTAGTCCGGAAAGAGTCATTAAGATGGCTATACAGAAAATGAGAACGGATATCGTAATCCATACTTCTTATGTTTGGGAGG
ATGATCTAGAACGTCTTACTCGTCTTAAAAATATGGTATACACCATAAAGTACGAACATGGGAAAAAAATGTTAATTAAAGTC
ATGCACGGCATATACAAAAACTTATTATACGGCGAAAGGGAAAAAGTCATGTTTCATTTAGCCAAGCTCTATGTTGCTCAAAA
TGCGGCCACCCAATTCAGAGACATTTGTAAGGACTGTTACAAACTGGATGTGGCACGGTTTAAACCGCGGTTTAAGCAACTAA
TATTAGACTGTTTAGAAATTGTTACTAAAAAATCTTGCTATAGTATCCTGGAAATCTTAGAAAAACATATTATTTCCCTATTT
ACTATGAAAGTTATGACTGAAGAAGAAAAAAACCTATGTTTAGAAATATTATATAAAGTAATTCATTATAAAACAATACAATG
TTAA
Tengani 62 MGF 505 2R (AY261364.1:28261-29830 )
SEQ ID No. 152
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCGTAGGAGACGACCACATACTCATACGGCGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCTTGCAGGGTGATCAATATGACCTGATCCATAAGTATGAAAACCAAATCGGCGACTTTCATCTTATCTT
ACCATTGATTCAAGACGCGAAAACGTTTGAAAAATGCCATGCTTTAGAACGTTTTTGTGGTGTTTCATGTCTGCTAAAACATG
CTACAAAATACAACATGCTCCCTGTTCTCCAAAAATACCAAGAAGAGCTGTCCATGAGAGCATATCTTTGCGAAACCCTATTT
GAACTAGCATGCCTATGGCAGAGGTATGATGTCCTTAAATGGATAGAGCAAACCATGCATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAATATCTCGAGAAGACAGCGGCCAAAGGGCTCCTCCACTTTGTGCTAGAAACGTTAAAATACGGCGGTAAC
ATAGATATTGTCCTGACCCAAGCCGTAAAGTACAATCATAGAAAACTTTTAGATTATTTTCTGCGTCAACTACCTCGTAAACA
TATTGAAAAGCTTTTGTTGCTGGCCGTGCAGGAAAAGGCTTCTAAAAAAACATTGAACTTACTGTTGTCACATTTAAACTACT
CCGTGAAACGCATCAAAAAACTGCTGCGCTATGTGATAGAGTACGAGTCCACCTTGGTGATAAAGATTTTATTAAAAAAAAGA
GTAAACCTGATAGATGCCATGTTGGAAAAGATGGTAAGATATTTTTCTGCGACGAAAGTGAGGACGATCATGGATGAGCTTTT
GATTAGTCCGGAAAGAGTCATTAAGATGGCTATACAGAAAATGAGAACGGATATCGTAATCCATACTTCTTATGTATGGGAGG
ATGATCTAGAACGACTTACTCGTCTTAAAGATATGGTATACACCGTAAAGTACGAACATGGGAAAAAAATGTTAATTAAAGTC
ATACACGGCATATACAAAAACTTATTATACGGCGAAAAAGAAAAGGTCATGTTTCATTTAGCCAAGCTCTATGTTGCTCAAAA
CGCGGCCACCCAATTCAGAGACATTTGTAAGGACTGTTACAAACTGGATGTGGCACGGTTTAAACCGCGGTTTAAGCAACTAA
TGTTAGACTGTTTAGAAATTGTTACTAAAAAATCTTGCTATAGTATTCTGGAAATCTTAGAAAAACATATGATTTCCCTATTT
ACTATGAAAGTTATGACTGAAGAAGAAAAAAACCTATGTTTAGAAATATTATATAAGTAA
Pretorisuskop/96/4 MGF 505 2R (AY261363.1:33795-35374)
SEQ ID No. 153
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCGTAGGAGACGACCACATCCTCATACGGCGGGATCTCATCCTTTCCACCA
ACGAGGCCTTAAGAATGGCGGGAGAAGAAGGAAACAATGAAGTAGTAAAGCTCTTGTTACTGTGGAAGGGAAATCTTCATTAC
GCCATCATAGGAGCCTTGCAGGGTGATCAATATGACCTGATCCATAAGTATGAAAACCAAATCGGCGACTTTCATCTTATCTT
ACCATTGATTCAAGATGCGAAAACGTTTGAAAAATGCCACGCTTTAGAACGTTTTTGTGGTGTTTCATGTCTGCTAGAACATG
CTACAAAATACAACATGCTCCCTATTCTCCAAACATACCAAGAAGAGCTGTCTATGAGAGCATATCTTCGCGAAACCCTATTT
GAACTAGCGTGCCTGTGGCAGAGGTATGATGTCCTTAAATGGATAGAGCAAACCATGCATGTTTACGACCTAAAGATTATGTT
TAATATTGCCATCTCCAAGAGGGATCTGACGATGTATTCCTTAGGATATATTCTTCTTTTTGATAGAGAGAACACCGAAGCTA
CGTTGTTAACACAACATCTCGAGAAGACAGCGGCCAAAGGGCTCCTCTACTTTGTGCTAGAAACGTTAAAATACGGCGGTAAC
ATAGATATCGTCCTGACTCAAGCCGTAAAGTACAATCATAGAAAACTTTTAGATTATTTTCTGCGTCAACTACCTCGTAAACA
TATTGAAAAGCTTTTGTTGCTGGCCGTGCAGGAAAAGGCTTCTAAAAAAACATTGAACTTACTGTTGTCCCATCTAAACTACT
CCGTGAAACGTATCAAAAAACTGCTGCGCTATGTGATAGAGTACGAGTCCACCTTGGTGATAAAGATTTTATTAAAAAAAAGA
GTAAACCTGATAGATGCCATGTTGGAAAAGATGGTAAGATATTTTTCTGCGACGAAAGTGAGGACGATCATGGATGAGCTTTT
GATTAGTCCGGAAAGAGTCATTAAGATGGCTATACAGAAAATGAGAACGGATATCGTAATCCATACTTCTTATGTTTGGGAGG
ATGATCTAGAACGACTTACTCGTCTTAAAAATATGGTATACACCATAAAGTACGAACATGGGAAAAAAATGTTAATTAAAGTC
ATGCACGGCATATACAAAAACTTATTATACGACGAAAGAGAAAAGGTCATGTTTCATTTAGCCAAGCTCTATGTTGCTCAAAA
CGCGGCCACCCAATTCAGAGACATTTGTAAGGACTGTTACAAACTGGATGTGGCACGGTTTAAACCGCGGTTTAAGCAACTAA
TGTTAGACTGTTTAGAAATTGTTACTAAAAAATCTTGCTATAGTATCCTGGAAATCTTAGAAAAACATATTATTTCCCTATTT
ACTATGAAAGTTATGACTGAAGAGGAAAAAAACCTATGTTTAGAAATATTATATAAGTAA
Warmbaths MGF 505 2R (AY261365.1:33029-34597)
SEQ ID No. 154
ATGTTTTCCCTTCAAGACCTTTGCCGAAAGCATCTTTTTATTCTTCCCGATGTTTTTGGCGAGCATGTATTACAACGATTAGG
ACTGTATTGGAGATGTCACGGCTCCCTTCAACGCGTAGGAGACGACCACATACTCATACGGCGGGATCTCATCCTTTCCACCA
ACGAGGCCTTAAGAATGGCGGGAGAAGAAGGAAACAATGAAGTAGTAAAGCTCTTGTTACTGTGGAAGGGAAATCTTCATTAC
GCCATCATAGGAGCCTTGCAGGGTGATCAATATGACCTAATCCATAAGTATGAAAACCAAATCGGCGACTTTCATCTTATCTT
ACCATTGATTCAAGATGCGAAAACGTTTGAAAAATGCCACGCTTTAGAACGTTTTTGTGGTGTTTCATGTCTGCTAAAACATG
CTACAAAATACAACATGCTCCCTATTCTCCAAACATACCAAGAAGAGCTGTCTATGAGAGTATATCTTCGCGAAACCCTATTT
GAACTAGCATGCCTATGGCAGAGGTATGATGTCCTTAAATGGATAGAGCAAACCATGCATGTTTACGACCTAAAGGTTATGTT
TAATATTGCCATCTCCAAGAGAGATCTGACTATGTATTCCTTAGGATATATTCTTCTTTTTGATAGAGAGAACACCGAAGCTA
CGTTGCTAACGCAACATCTCGAGAAAACAGCGGCCAAGGGGCTCCTCCACTTTGTGCTAGAAACGTTAAAATACGGCGGTAAC
ATAGATATCGTCCTGTCCCAAGCCGTAAAGTACAATCATAGAAAACTTTTAGATTATTTTCTGCGTCAACTACCTCGTAAAAA
TATTGAAAAACTTTTGTTGCTGGCTGTTCAGGAAAAGGCTTCTAAAAAAACATTGAACTTACTGTTGTCCCATTTAAACTACT
CCGTGAAACGCATCAAAAAACTGCTGCGCTATGTGATAGAGTACGAGTCCACCTTGGTGATAAAGATTTTATTAAAAAAAAGA
GTAAACCTGATAGATGCCATGTTGGAAAAGATGGTAAGATATTTTTCTGCGACGAAAGTGAGGACGATCATGGATGAGCTTTC
GATTAGTCCGGAAAGAGTCATTAAGATGGCTATACAGAAAATGAGAACAGATATCGTAATCCATACTTCTTATGTTTGGGAGG
ATGATCTAGAACGACTTACTCGTCTTAAAAATATGGTATACGCCATAAAGTATGAACATGGGAAAAAAATGTTAATGAAAGTC
ATGCACGGCATATACAAAAACTTATTATACGGCGAAAGAGAAAAGGTCATGTTTCATTTAGCCAAGCTCTATGTTGCTCAAAA
CGCGGCCACCCAATTCAGAGACATTTGTAAGGACTGTTACAAACTGGATGTGGCACGGTTTAAGCAACTAACGTTAAACTGTT
TAGAAATTATTACTAAAAAATCTTGCTATAGTATCCTGGAAATCCTAGAAAAACATATTATTTCCCTATTTACTATGAAAGTT
ATGACTGAAGAAGAAAAAAACCTATGTTTAGAAATATTATATAAAGTAATTCATTATAATACAATACAATGTTAA
Malawi Lil-20/1 (1983) MGF 505 2R (AY261361.1:31541-33121)
SEQ ID No. 155
ATGTTTTCCCTTCAAGATCTTTGCCGAAAGCATCTTTTTATTCTTCCCGATGTTTTTGGCGAGCATGTACTACAACAGTTAGG
ACTGTACTGGAAACGTCACGGCTCTCTTCAACGCATAGGAGACGACCACATACTCATACGGCGGGACCTCATCCTTTCCACCA
ACGAGGCCTTAAAAATGGCGGGAGAGGAAGGAAACAATGAAGTAGTAAAGCTCTTGTTACTGTGGGAGGGAAACCTTCATTAT
GCCATCATAGGGGCCTTACAGGGTGATCAATATGATCTGATCCATAAGTATGAAAACCAAATCGAAGACTATCATCATATCTT
GCCATTGATTCAAGATGCGAAAACGTTTGAAAAATGCCACGCCTTAGAACGTTTTTGTGATGTTCCATGTCTGCTAGAACATG
CTACAAAACACAACATGCTCCCTATTCTCCAAAAATACCAAGAAGAGCTGTCTATAAGAGTGTATCTTCGCGAAACCCTATTC
GAACTAGCATGCCTATGGCAGAGGTATGATGTTCTTAAATGGATAGAGCAAACCATGCATGTTTACGACCTAAAGATTATGTT
TAATATTGCCATCTCCAAGAGGGATCTAAGCATGTACTCCTTAGGATATGTTCTCCTTTTTGATAGAGGAAACATCGAAGCTA
CGTTCCTAACGCAACATCTCGAGAAGACAGCGGCCAAAGGGCTCCTCCACTTTGTGCTAGAAACGTTAAAATACGGCGGTAAC
TTAAATATCGTCCTGTCCCAAGCCGTAAAATACAATCATAGAAAACTTTTGGATTATTTTCTGCGTCAACTACCTCGTAAAAA
TATTGAAAAACTTTTGTTGCTGGCCGTGCAGGAAAAGGCTTCTAAAAAAACATTGAACCTACTGTTGTCACACTTAAACTACT
CCGTGAAACACATCAAAAAACTGCTGCGCTATGTGATAGAGTACGAGTCTACCTTGGTGATAAAACTTTTATTGAAAAAAAGA
GTAAACCTGATAGACGCCGTGTTGGAAAAGAATGTAAGATATTTTTCTGCGATTAAAGTGAGGACTATTATGGATGAGCTTTC
GATTAGTCCGGAAAGAGTCATTAAGATGGCCATACAGAAAATGAGAACGGATATTGTGATTCAGACTTCTTATATTTGGGAGG
ATGATCTAGAAAGACTTATTCGTCTTAAAAATATGGTATACACCATAAAGTATGAACATGGGAAAAAAATGTTAATTAAAGTT
ATTCACGGCATATACAAAAACTTATTATACGGCGAAAAAGAAAAAGTCATGTTTCATTTAGCCAAACTCTATGTTGCTCAAAA
CGCGGCCACCCAATTCAGGGACATTTGTAAGGACTGTTGCAAACTGGATGTGGCGCGGTTTAAACCGCGGTTTAAACAACTAA
TTTTAGACTGTTTAGAAATGGTTACTAAAAAATCTTGCTTTAGTATTATAGAAATCTTAGAAAACTATATTATTTCCCTATTT
GTGATGAAAGTCATCACTGAAGAAGAAAAAAACCTATGTTTAGAACTATTATATAAAGTAATTAGTTACAAAACGATATAATG
TTAA
Ken05/Tk1 MGF 505 2R (NC_044945.1:35094-36674)
SEQ ID No. 156
ATGTTTTCCCTTCAAGACCATTGCCGAAAGCATCTTTTTATTCTTCCCGATGTTTTTGGCGAGCATGTACTACAACGGTTAGG
ACTGTACTGGGGACGTCACGGCTCTCTTCAACGAATCGGGGACGATCACATACTCATACGGCGGGACCTCATCCTTTCCACCA
ACGAGGCCTTAAAAATGGCGGGAGAAGAAGGAAACAATGAAGTAGTAAAGCTCTTGTTACTGTGGGAGGGAAATCTTCATTAT
GCTATCATAGGGGCTTTACAGGGTGATCAATATGACCTAATCCATAAGTATGAAAACCAAATCGAAGACTATCATCATATCTT
ACCATTGATTCAAGATGCGGAAACGTTTGAAAAATGCCACGCCTTAGAACGTTTTTGTGATGTTCCATGTCTGCTAGAACATG
CTACAAAACACAACATGCTCCCTATTCTCCAAAAATATCAAGAAGAGTTGTCTATAAGAGTGTATCTACGCGAAACCCTATTC
GAACTAGCATGCCTATGGCAGAGGTATGATATTCTTAAATGGATAGAGCAAACCATGCATGTTTACGATCTAAAAATTATATT
TAATATTGCCATCTCCAAGAGGGATCTAAGCATGTACTCCTTAGGATATGTTCTCCTTTTTGATAGAGGGAACACCGAAGCTA
CCTTGTTAACGCAACACCTCGAGAAGACAGCGGCCAAGGGGCTCCTCCACTTTGTGCTAGAAACGTTAAAATACGGCGGTAAC
TTAAATATCGTCCTGTTCCAAGCCGTAAAATACAATCATAGAAAACTTTTAGATTATTTTCTGCGTCAACTACCCCGTAAAAA
TATTGAAAAACTTTTGTTGCTGGCTGTGCAGGAAAAAGCTTCTAAGAAAACATTGAACTTACTGTTGTCACATTTAAACTACT
CCGTGAAACGCATCAAAAAACTGCTGCGCTATGTGATAGAGTACGAGTCTACCTTGGTGATAAAGATTTTATTAAAAAAAAGA
GTAAACCTAATAGACGCCGTGTTGGAAAAGACTGTAAGATATTTTTCTGAGACGAAAGTAAAGACTATTATGGATGAGCTTTC
GATTAATCCGGAAAAAGTCATTAAGATGGCCATACAGAAAATGAGAACGGATATTGTGATCCAAACTTCTTATATTTGGGAGG
ATGATCTAGAAAGACTTATTCGTCTTAAAAATATGGTATACACCATAAAGTATGAACATGGGAAAAAAATGTTAATGAAAGTT
ATTCACGGCATATACAAAAACTTATTACACGATGAAAAAGAAAAAGTCATGTTTCATTTAGCCAAGTTCTATATTGCTCAAAA
CGCGGCCACCCAATTCAGAGACATTTGTAAAGACTGTTGCAAACTGGATGTGGCGCGGTTTAAACCGCGGTTTAAACAACTAA
TTTTAGACTGTTTAGAAATTGTTACTAAAAAATCTTGCTTTAGTATTATAGAAATTTTAGAAAACCATATTATTTCCCTATTT
ATGATGAAAGTTATCACTGAAGATGAAAAAAACCTAGGTTTAGAATTATTATATAAAGTAATTAGTTACAAAATGATATCATA
TTAA
Kenya 1950 MGF 505 2R (AY261360.1:37419-38999)
SEQ ID No. 157
ATGTTTTCCCTTCAAGACCTTTGCCGAAAGCATCTTTTTATTCTTCCCGATGTTTTTGGCGAGCACGTACTACAACAATTGGG
GCTGTACTGGGAAAAACACGGCTCTCTTCAACGAATAGGGAACGATCACATACTCATACGGCGGGATCTCATCCTTTCTATCA
ACGAGGCCTTAAAAATAGCGGCAGAGGAAGGAAACAATGAAGTAGTAAAGCTTTTGTTACTATGGAAGGGAAATCTTCATTAT
GCCATCATAGGAGCTTTGGAGGGTGACCAATATGACCTGATCTATACGTATGAAAACCAAATTGAAGACTATCATCATATCTT
GCCATTGATTCAAGATGCGAAAACGTTTGAAAAATGCCACGCCTTGGAACGTTTTTGTGATGTTCCATGCCTGCTAGAGCATG
CTATAAAACACAACATGCTCCCTATTCTCCAAAAATATCAAGAAGAACTGTTTATAAGAGTGTATCTCCGCGAAACCCTATTT
GAACTAGCATGCCTATGGCAGAGGTATGATATTCTTAAATGGATAGAGCAAACCATGCATGTTTACGATCTAAAAATTATATT
TAATATTGCCATCTCCAAGAGGGATCTAAGCATGTACTCCTTAGGATATGTTCTCCTTTTTGATAGAGGGAACACCGAAGCTA
CCTTGTTAACGCAACACCTCGAGAAGACAGCGGCCAAGGGGCTCCTCCACTTTGTGCTAGAAACGTTAAAATACGGCGGTAAC
ATAAACATCGTCCTATCCCAAGCCGTAAAATACAATCATAGGAAACTTTTAGATTATTTTCTGCGTCAACTACCTCGTAAAAA
TATTGAAAAACTTTTGTTGTTAGCCGTACAGGAAAAGGCTTCTAAGAAAACATTGAACTTACTGTTGTCTTATTTAAACTACT
CCGTGAAACGCATCAAAAAACTGCTGCGCTATGTGATAGAGTACGAGTCCACACTGGTGATAAGGATTTTATTAAAAAAAAGA
ATAAACCTAATAGACGCCGTGTTGGAAAAGACTGTAAGATATTTTTCTGAGACGAAAGTAAAGACTATTATGGATGAGCTTTC
GATTAATCCGGAAAAAGTTATTAAGATGGCCATACAGAAAATGAGAACGGATATTGTGATCCAAACTTCTTATATTTGGGAGG
ATGATCTAGAAAGACTTATTCGTCTTAAAAATATGGTATACACCATAAAGTATGAACATGGGAAAAAAATGTTAATGAAAGTT
ATTCACGGCATATACAAAAACTTATTACACGATGAAAAAGAAAAAGTCATGTTTCATTTAGCCAAGTTCTATATTGCTCAAAA
CGCGGCCACCCAATTCAGAGACATTTGTAAAGACTGTTGCAAACTGGATGTGGCGCGGTTTAAACCGCGGTTTAAACAACTAA
TTTTAGACTGTTTAGATATTATTACTAAAAAAACGTGCCTCAATATTATGGAAATCTTAGAAAATCATATTATTTCGCTATTT
GCTATGAAAATGATGACTGAAGATGAAAAAAACCTAGGTTTAGAATTATTATATAAAGTAATTAGTTACAAAATGATATCATA
TTAA
Ken06.Bus MGF 505 2R (NC_044946.1:31225-32799)
SEQ ID No. 158
ATGTTTTCCCTTCAAGACCTTTGCCGAAAGCATCTTTTTATTCTTCCCGATGTTTTTGGCGAGCATGTACTACAACAATTGGG
GCTGTACTGGGAAAAACACGGCTCTCTTCAACGAATAGGGAACGATCACATACTCATACGGCGGGATCTCATCCTTTCTATCA
ACGAGGCCTTAAAAATAGCGGCAGAGGAAGGAAACAATGAAGTAGTAAAGCTCTTGTTACTGTGGAAGGGAAATCTTCATTAT
GCCATCATAGGAGCCTTGCAGGGTGACCAATATGACCTCATCCATACGTACGAAAACCAAATCGAAGACTATCATCATATCTT
GCCATTGATTCAAGATGCGAAAACGTTTGAAAAATGCCACGCCTTGGAACGTTTTTGTGATGTTCCATGCCTACTAGAACATG
CTACAAAACACAACATGCTCCCTATTCTCCAAAAATACCAAGAAGAGCTGTTTATAAGAGTGTATCTCCGCGAAACCCTATTT
GAACTAGCATGCCTATGGCAGAGGTATGATATCCTTAAATGGATAGAGCAAACCATGCATGTTTACGACCTAAAGATTATGTT
TAATATTGCCATCTCCAAGAGGGATCTAAGCATGTACTCTTTAGAATATATTCTCTTTTTTAATAGAGGGAACACCGATGTTG
CGTTAGCAACGTTGCTAACGCAACATCTCGAGAAGACAGCGGCCAAGGGGCTCCTCCACTTTGTGCTAGAAACGTTAAAATAC
GGCGGTAACATAAACATCGTCCTCTCCCAAGCCGTAAAATACAATCATAGAAAACTTTTAGATTATTTTCTGCGTCAACTACC
TCGTAAAAATATTGAAAAACTTTTGTTGCTGGCCGTACAGGAAAGGGCTTCTAAGAAAACATTGAACTTACTGTTGTCTTATT
TAAACTACTCCGTGAAACGCATCAAAAAACTGCTGCGCTATGTGATAGAGCATGAGTCCACACTGGTGATAAGGATTTTATTA
AAAAAAAGAATAAACCTAATAGACGCCGTGTTGGAAAAGACTGTAAGATATTTTTCTATGACTAAAGTGAGGACGATCATGGA
TGAGCTTTCGATTAATCCGGAAAAAGTCATTAAAATGGCCGTGCAGAAAATGAGAACGGATATCGTGATCCATACTTCTTATG
TTTGGGAGGATGATCTAGAAAGACTTATTCGTCTTAAAAATATGCTATACACTATAAAGTATGAGCATGGAAAAAAAATGCTA
ATTAAAGTCATTCACGGCATATACAAAAACTTATACGGCGAAAAAGAAAAAGTCATGTTTAATTTGGCCAAGTTCTATGCTGC
TCAAAACGCGGCCACCCAATTCAAAGACACTTGTAAAGACTGTTGCAAACTGGATGTGGCGCGGTTTAAACAACTAATTTTAG
ACTGTTTAGACATTATTACTAAAAAAACGTGCCTCAGTATCATGGAAATCTTAGAAAATCATATTATTTCGCTATTTGCTATG
AAAATGATGACGGAGGATGAAAAAAACTTAGGTTTAGAAATATTATATAAAGTAATTAGTTACAAAATGATATCATATTAA

In an embodiment the attenuated ASFV of the invention comprises a functional version of MGF 505 2R. Suitably the functional version of MGF 505 2R comprises the sequence of 25 SEQ ID No. 147, 148, 149, 150, 151, 152, 153, 154, 155, 156, 157 or 158. Suitably the functional version of MGF 505 2R comprises a sequence having at least 70%, at least 80%, at least 90% or at least 95% identity with SEQ ID No. 147, 148, 149, 150, 151, 152, 153, 154, 155, 156, 157 or 158. Suitably the functional version of MGF 505 2R consists of the sequence of SEQ ID No. 147, 148, 149, 150, 151, 152, 153, 154, 155, 156, 157 or 158.

Benin 97/1 MGF 505 6R
SEQ ID No. 257
ATGTTCTCCCTACAGGACCTCTGTCGGAAGAACACTTTCTTCCTT
CCAAATGATTTTAGCAAGCATACTCTACAACGGCTGGGGTTGTAT
TGGAAAGAGCATGGATCCGTCCATCGAATAGAAAAGGACAGCATA
ATGATACAGAATGAATTGGTTCTTTCTATCAATGATGCTTTACAG
CTTGCAGGAGAGGAGGGGGACACAGATGTGGTACAGCTCTTGTTA
CTATGGGAGGGAAATCTGCATTATGCCATCATAGGAGCTTTGAAG
ACTGAGAATTATAATCTAGTATGTGAGTACCATAGCCAAATTCAG
GACTGGCATATTCTCCTACCCTTGATTCAAGATCCAGAAACATTC
GAAAAATGTCATGATTTAAGCCTTGGATGTGACCTTATATGCCTT
CTCCAACATGCTGTAAAATGTGATATGCTTTCTATTCTTGTTAAA
TATAAGGAGGATTTACTAAATGTAAGGATTAGGCATCGTACCCAA
TCCCTGTTTGTTTTGGCATGCGAAAATCGGAGATTTGAGATTATT
GAATGGATAGGTCAAAATCTGTCAATTCCTGAACCTGAGGCCATT
TTTAGCATTGCTATTGTTACAAAAGATGTAGAACTGTTTTCCTTA
GGATATAAAATTATTTTTGATTACATGCAAAGACAAGGAATTTTT
CAATTAACCAATGTAGTTCGCATGCTTCTGCTAAATCGTCATATT
GGTATGGCAATAGAAAAAGGACTTTTACCTTTTATTCTGGAAACT
TTAAAATATGGTGGTAGTGTAAAAAGAGCTTTATCTTATGCAGTA
ATAGATAATAAAAGAAAAATTATAGACTATCTTGTACGCCATGAA
AATATACCCCGAGGAACTATTGAAAGACTTTTGCATCTAGCTGTG
AAAAAACAATCTTCCAGGAAAACTTTGAACTTGTTGCTATCTTAC
ATAAATTACAAGGTGAAAAATGTTAAAAAGCTGGTAGAGCATGTA
GTAGATCACAAATCCACTCTTGTGTTAAAAATTTTATTGGAAAAA
AAGGAAAATCTAGTGGATGCTGTTTTAACAAGACTTGTAAAACAT
TCTACATATTTCCAGGTGAGAGAATTTATCCAGGAGTTTTCCATC
AGCCCAGAAAAATTCATTAAAATAGCTGTGCGGGAAAAGAAAAAT
GTGTTAATCGAGGCTATTTCTGAAGATATTTGGGAAAATCCCACA
GAAAGAATTACTTATCTCAAACAGATAGTGCACACCATAAAATAT
GAAAGTGGAAGGCGGTTTTTGATAGACATCATTCACAGCATTTAC
CAAAGTTACTCACTAAAACACGAAGATATTCTTAAACTGGCAACA
TTTTATGTCAAACACAATGCAATCACCCATTTTAAAGATCTCTGC
AAATATCTTTGGCTGAACAGAGGAACAGAAAGTAAGAAACTGTTT
TTAGAGTGCTTGGAAATTGCTGATGAGAAGGAGTTTCCTGATATT
AAAAATATTGTGAGTGAATATATTAACTACTTGTTTACTGCAGGA
GCTATTACCAAGGAAGAAATCATGCAAGCTTATGCTTTAGAGTAT
GACATGTATTAA
L60 MGF 505 6R
SEQ ID No. 258
ATGTTCTCCCTACAGGACCTCTGTCGGAAGAACACTTTCTTCCTT
CCAAATGATTTTAGCAAGCATACTCTACAACGGCTGGGGTTGTAT
TGGAAAGAGCATGGATCCGTCCATCGAATAGAAAAGGACAGCATA
ATGATACAGAATGAATTGGTTCTTTCTATCAATGATGCTTTACAG
CTTGCAGGAGAGGAGGGGGACACAGATGTGGTACAGCTCTTGTTA
CTATGGGAGGGAAATCTGCATTATGCCATCATAGGAGCTTTGAAG
ACTGAGAATTATAATCTAGTATGTGAGTACCATAGCCAAATTCAG
GACTGGCATATTCTCCTACCCTTGATTCAAGATCCAGAAACATTC
GAAAAATGTCATGATTTAAGCCTTGGATGTGACCTTATATGCCTT
CTCCAACATGCTGTAAAATGTGATATGCTTTCTATTCTTGTTAAA
TATAAGGAGGATTTACTAAATGTAAGGATTAGGCATCGTACCCAA
TCCCTGTTTGTTTTGGCATGCGAAAATCGGAGATTTGAGATTATT
GAATGGATAGGTCAAAATCTGTCAATTCCTGAACCTGAGGCCATT
TTTAGCATTGCTATTGTTACAAAAGATGTAGAACTGTTTTCCTTA
GGATATAAAATTATTTTTGATTACATGCAAAGACAAGGAATTTTT
CAATTAACCAATGTAGTTCGCATGCTTCTGCTAAATCGTCATATT
GGTATGGCAATAGAAAAAGGACTTTTACCTTTTATTCTGGAAACT
TTAAAATATGGTGGTAGTGTAAAAAGAGCTTTATCTTATGCAGTA
ATAGATAATAAAAGAAAAATTATAGACTATCTTGTACGCCATGAA
AATATACCCCGAGGAACTATTGAAAGACTTTTGCATCTAGCTGTG
AAAAAACAATCTTCCAGGAAAACTTTGAACTTGTTGCTATCTTAC
ATAAATTACAAGGTGAAAAATGTTAAAAAGCTGGTAGAGCATGTA
GTAGATCACAAATCCACTCTTGTGTTAAAAATTTTATTGGAAAAA
AAGGAAAATCTAGTGGATGCTGTTTTAACAAGACTTGTAAAACAT
TCTACATATTTCCAGGTGAGAGAATTTATCCAGGAGTTTTCCATC
AGCCCAGAAAAATTCATTAAAATAGCTGTGCGGGAAAAGAAAAAT
GTGTTAATCGAGGCTATTTCTGAAGATATTTGGGAAAATCCCACA
GAAAGAATTACTTATCTCAAACAGATAGTGCACACCATAAAATAT
GAAAGTGGAAGGCGGTTTTTGATAGACATCATTCACAGCATTTAC
CAAAGTTACTCACTAAAACACGAAGATATTCTTAAACTGGCAACA
TTTTATGTCAAACACAATGCAATCACCCATTTTAAAGATCTCTGC
AAATATCTTTGGCTGAACAGAGGAACAGAAAGTAAGAAACTGTTT
TTAGAGTGCTTGGAAATTGCTGATGAGAAGGAGTTTCCTGATATT
AAAAGTATTGTGAGTGAATATATTAACTACTTGTTTACTGCAGGA
GCTATTACCAAGGAAGAAATCATGCAAGCTTATGCTTTAGAGTAT
GACATGTATTAA
China/2018/AnhuiXCGQ MGF 505 6R
SEQ ID No. 259
ATGTTCTCCCTACAGGACCTCTGTCGGAAGAACATTTTCTTCCTT
CCAAATGATTTTAGCAAGCATACCCTACAATGGCTGGGATTATAT
TGGAAAGAGCATGGATCCGTCCATCGAGCAGAAAAAGACAGCATA
ATGATACAGAATGAATTGGTTCTTTCTATCAATGATGCTTTACAG
CTTGCAGGAGAGGAGGGGGACACAGATGTAGTACAGCTCTTGTTA
TTATGGGAGGGAAATCTGCATTATGCCATCATAGGAGCCTTGAAG
ACTGAAAAATATAACCTAATATGTGAGTATCATAGCCAAATTCAG
GACTGGCATATTCTCCTACCCATGATTCAAGATCCAGAAACATTC
GAAAAATGTCATGATTTAAGCCTTGGATGTGACTTTATTTGCCTT
CTCCAACATGCTGTAAAATACAACATGCTTTCTATTCTTGTCAAA
TATAAGGAGGATCTACTAAATGCAAGGATTAGGCATCGTATCCAA
TCCCTGTTTGTTTTGGCATGCGAAAATCGGAGAATTGAAATTATT
GATTGGATAGGCCAAAATCTGCCAATTCCTGAACCTGATGCCATT
TTTAGCATTGCTGTTGCTACAAGAGATTTAGAACTGTTTTCCTTA
GGGTACAAGATTATTTTTGATTACATGCAAAGACAGGGAATCATT
CAATTAACCAATGGAGTTCGCATGGTTGTGCTAAATCGTCACATT
AGCATGGCAATAGATAATGGTCTTTTACCTTTTGTTCTGGAAACT
TTAAAACATGGTGGGAATATACATAGAGCCTTATCTTATGCAGTA
ACACACAATAGAAGAAAAATTCTGGATTATCTTATTCGCCAGAAA
AATATAGCCCCTAATACAATTGAAAGACTTTTATATCTGGCCGTG
AAAAATCAATCTTCCAGGAAAACTTTGAACTTGTTGCTATCTTAC
ATAAATTACAAGGTGAAAAATGTTAAAAAGCTGGTAGAGCATGTA
GTAAATGAGAAATCCACTCTTGTGTTAAAAATTTTATTAGAAAAA
AAGGAAAATCTAGTGGATGCTGTTTTAACAAGACTTGTAAAACAT
TCTACATATTTCCAGGTGAGAGAATTTATCCAGGAGTTTTCCATC
AGCCCAGAAAAATTCATTAAAATAGCTGTGCGGGAAAAGAAAAAT
GTGTTAATCGAGGCTATTTCTGAAGATATTTGGGAAAATCCCACA
GAAAGAATTACTTATCTCAAACAGATAGTGCACACCATAAAATAT
GAAAGTGGAAGGCGATTTTTGGTAGACATCATTCACAGCATTTAC
CAAAGTTACTCACTAAAACACGAAGATATTCTTAAACTGGCAACA
TTTTATGTCAAACACAATGCAATCACCCATTTTAAAGACCTCTGC
AAATATCTTTGGCTGAACAGAGGAACAGAAAGTAAGAAACTGTTT
TTAGAGTGTTTAGAAATTGCTGATGAGAAGGAGTTTCCTGATATT
AAAAGTATTGTGAGTGAATATATTAACTACTTGTTTACTGCAGGA
GCTATTACCAAGGAAGAAATCATGCAAGCCTATGATGCTTTAGAG
TAG
Georgia 2007/1 MGF 505 6R
SEQ ID No. 260
ATGTTCTCCCTACAGGACCTCTGTCGGAAGAACATTTTCTTCCTT
CCAAATGATTTTAGCAAGCATACCCTACAATGGCTGGGATTATAT
TGGAAAGAGCATGGATCCGTCCATCGAGCAGAAAAAGACAGCATA
ATGATACAGAATGAATTGGTTCTTTCTATCAATGATGCTTTACAG
CTTGCAGGAGAGGAGGGGGACACAGATGTAGTACAGCTCTTGTTA
TTATGGGAGGGAAATCTGCATTATGCCATCATAGGAGCCTTGAAG
ACTGAAAAATATAACCTAATATGTGAGTATCATAGCCAAATTCAG
GACTGGCATATTCTCCTACCCATGATTCAAGATCCAGAAACATTC
GAAAAATGTCATGATTTAAGCCTTGGATGTGACTTTATTTGCCTT
CTCCAACATGCTGTAAAATACAACATGCTTTCTATTCTTGTCAAA
TATAAGGAGGATCTACTAAATGCAAGGATTAGGCATCGTATCCAA
TCCCTGTTTGTTTTGGCATGCGAAAATCGGAGAATTGAAATTATT
GATTGGATAGGCCAAAATCTGCCAATTCCTGAACCTGATGCCATT
TTTAGCATTGCTGTTGCTACAAGAGATTTAGAACTGTTTTCCTTA
GGGTACAAGATTATTTTTGATTACATGCAAAGACAGGGAATCATT
CAATTAACCAATGGAGTTCGCATGGTTGTGCTAAATCGTCACATT
AGCATGGCAATAGATAATGGTCTTTTACCTTTTGTTCTGGAAACT
TTAAAACATGGTGGGAATATACATAGAGCCTTATCTTATGCAGTA
ACACACAATAGAAGAAAAATTCTGGATTATCTTATTCGCCAGAAA
AATATAGCCCCTAATACAATTGAAAGACTTTTATATCTGGCCGTG
AAAAATCAATCTTCCAGGAAAACTTTGAACTTGTTGCTATCTTAC
ATAAATTACAAGGTGAAAAATGTTAAAAAGCTGGTAGAGCATGTA
GTAAATGAGAAATCCACTCTTGTGTTAAAAATTTTATTAGAAAAA
AAGGAAAATCTAGTGGATGCTGTTTTAACAAGACTTGTAAAACAT
TCTACATATTTCCAGGTGAGAGAATTTATCCAGGAGTTTTCCATC
AGCCCAGAAAAATTCATTAAAATAGCTGTGCGGGAAAAGAAAAAT
GTGTTAATCGAGGCTATTTCTGAAGATATTTGGGAAAATCCCACA
GAAAGAATTACTTATCTCAAACAGATAGTGCACACCATAAAATAT
GAAAGTGGAAGGCGATTTTTGGTAGACATCATTCACAGCATTTAC
CAAAGTTACTCACTAAAACACGAAGATATTCTTAAACTGGCAACA
TTTTATGTCAAACACAATGCAATCACCCATTTTAAAGACCTCTGC
AAATATCTTTGGCTGAACAGAGGAACAGAAAGTAAGAAACTGTTT
TTAGAGTGTTTAGAAATTGCTGATGAGAAGGAGTTTCCTGATATT
AAAAGTATTGTGAGTGAATATATTAACTACTTGTTTACTGCAGGA
GCTATTACCAAGGAAGAAATCATGCAAGCCTATGATGCTTTAGAG
TAG
Mkuzi 1979 MGF 505 6R
SEQ ID No. 261
ATGTTCTCCCTACAGGACCTCTGTCGGAAGAACACTTTCTTCCTT
CCAAATGATTTTAGCAATCATACCCTACAACGGCTGGGGTTATAT
TGGAAAGAGCATGGATCCGTCCATCGAATAGAAAAGGACAGCATA
ATGATACAGAATGAATTGGTTCTTTCTATCAATGATGCTTTACAT
CTTGCAGGAGAGGAGGGGAACACAGATGTAGTACAGCTCTTGTTA
TTATGGGAGGGAAATCTGCATTATGCCATCATAGGAGCCTTGAAG
ACTGAGAAATATAACCTAATATGTGAGTACCATAGCCAAATTCAG
AACTGGCATGTTCTCCTCCCCTTGATTCAAGATCCAGAAACATTC
GAAAAATGTCATGATTTAAGCCTTGGATGTGACTTTATTTGCCTT
CTCCAACATGCTGTAAAATGTGACATGCTTTCTATTCTTGTTAAA
TATAAGGAGGATCTACTAAATGCAAGGATTAGGCATCGTATCCAA
TCCCTGTTTGTTTTGGCATGCGAAAATCGGAGATTTGAGATTATT
GAATGGATAGGTCAAAATCTGCCAATTCCTGAACCTGAGGCCATT
TTTAGCATTGCTATTGTTACAAAAGATATAGAACTGTTTTCCTTA
GCGTACAAGCTTATTTTTGATTACATGCAAAGACAAGGAACTTTT
CAATTAACCAATATGGTCCGCATGCTTCTGCTAAATCGGTACATT
GGTATGGCAATAGAAAAAGGACTTTTACCCTTTATTCTGGAAACT
TTAAAATATGGTGGTAGTGTAAATAGAGCTTTATCTTATGCAGTA
ATAGATAATAAAAGAAAAATTATAGACTATCTTGTACGCCATGAA
AATATACCCCGTGGAACTATTGAAAGACTTTTGCATCTAGCTGTG
AAAAAACAATCTTCCAGAAAAACTTTGAACTTGTTGCTATCTTAC
ATAAATTACAAGGTGAAAAATGTTAAAAAGCTGGTAGAGCATGTA
GTAGATCACAACTCTACTCTTGTGTTAAAAATTTTATTGGAAAAA
AAGGAAAATCTAGTGGATGCTGTTTTAACAAGACTTGTAAAACAT
TCTACATATTTCCGGGTAAGAGAATTTATCCAGGAGTTTTCCATC
AGCCCAGAAAAATTCATTAAAATAGCTGTGCGGGAAAAGAAAAAT
GTGTTAATCGAGGCTATTTCTGAAGATATTTGGGAAAATCCCACA
GAAAGAATTACTTATCTCAAACAGATAGTGCACACCATAAAATAT
GAAAGTGGAAGGCGGTTTTTGATAGACATCATTCACAGCATTTAC
CAAAGTTATTCACTAAAACATGAAGATATTTTTAAACTGGCAACA
TTTTATGTCAAACACAATGCAATCACCCATTTTAAAGATCTCTGC
AAATATCTTTGGCTGAACAGAGGAACAGAAAGTAAGAAACTGTTT
TTAGAGTGCTTGGAAATTGCTGATGAGAAGGAGTTTCCTGATATT
AAAAGCATTGTGAGTGAATATATTAACTACTTGTTTACTGCAGGA
GCTATTACCAAGGAAGAAATCATGCAAGCCTATAATGCTTTAGAG
TAG
Pretorisuskop/96/4 MGF 505 6R
SEQ ID No. 262
ATGTTCTCCCTTCAGGACCTCTGTCGGAAGAACATTTTCTTCCTT
CCAAACGATTTTAGCAAGCATACCCTACAATGGCTGGGGTTATAT
TGGAAAGAGCATGGATCCGTCCATCGAGCAGAAAAAGACAGCATA
ATGATACAGAATGAATTGGTTCTTTCTATCAATGATGCTTTACTG
CTTGCAGGAGAGGAGGGGGACACAGATGTAGTACAGCTCTTGTTG
CTATGGGAAGGAAATCTGCATTATGCCATCATAGGAGCCTTGAAG
ACTGAGAAATATAGCCTAGTATGTGAGTACCATAGCCAAATTCAG
GACTGGCATGTTCTCCTCCCCTTGATTCAAGATCCAGAAACATTC
GAAAAATGTCATGATTTAAGCCTTGAATGTGACTTTATATGCCTT
CTACAACATGCTGTAAAATGTGACATGCTTTCTATTCTTGTTAAA
TATAAAGAGGATCTACTAAATGTAAGGATTAGGCATCGTATCCAA
TCCCTGTTTGTTTTGGCATGCGAAAATCGGAGATTTGAGATTATT
GAATGGATAGGTCAAAATCTGCCAATTCCTGAACCTGAGGCCATT
TTTAGCATTGCTATTGTTACAAAAGATATAGAACTGTTTTCCTTA
GGATACAAGCTTATTTTTGATTACATGCAAAGACAAGGAACTTTT
CAATTAACCAATATGGTTCGCATGCTTCTGCTAAATCGTTACATT
GGTATGGCAATAGAAAAAGGCCTTTTACCCTTTATCGTGGAAACT
TTAAAATATGGTGGTAGTGTAAATAGAGCTTTATCTTATGCAGTC
ATAGATAATAAAAGAAAAATTATAGACTATCTTGTACGTCATGAA
AATATACCCCGTGGAACTATTGAAAGACTTTTGCATCTAGCTGTG
AAAAAACAATCTTCCAGAAAAACTTTGAACTTGTTGCTATCTTAC
ATAAACTACAAGGTGAAAAATGTTAAAAAGCTGGTAGAGCATGTA
GTAGATCACAACTCTACCCTTGTGTTAAAAATTTTATTGGAAAAA
AAGGAAAATTTAGTGGATGCTGTTTTAACAAGACTTGTAAAACAT
TCTACATATTTCCGGGTGAGAGAATTTATCCAGGAGTTTTCCATC
AGCCCAGAAAAATTCATTAAAATAGCTGTGCGGGAACAGAAAAAT
GTGTTAATCGAGGCTATTTCTGAAGATATTTGGGAAAATCCCACA
GAAAGAATTACTTATCTCAAACAGATAGTGCACACCATAAAATAT
GAAAGTGGAAGGCGGTTTTTGATAGACATCATTCACAGCATTTAC
CAAAGTTACTCACTAAAACACGAAGATATTCTTAAACTGGCAATA
TTTTATGTCAAATACAATGCAATCACCCATTTTAAAGATCTCTGC
AAATATCTTTGGCTGAACAGAGGAACAGAAAGTAAGAAACTGTTT
TTAGAGTGCTTGGAAATTGCTGATGAGAAGGAGTTTCCTGATATT
AAAAGCATTGTGAGTGAATATATTAACTACTTGTTTACTGCAGGA
GCTATTACTAAGGAAGAAATCATATAA
Tengani 62 MGF 505 6R
SEQ ID No. 263
ATGTTCTCCCTTCAGGACCTCTGTCGGAAGAACACTTTCTTCCTT
CCAAATGATTTTAGCAAGCATACCCTACAACGGCTGGGGTTATAT
TGGAAAGAGCATGGATCCGTCCATCGAGCAGAAAAAGACAGCATA
ATGATACAGAATGAATTGGTTCTTTCTATCAATGATGCTTTACAG
CTTGCAGGAGAGGAGGGGGACACAGATGTAGTACAGCTCTTATTA
TTATGGGAAGGAAATCTTCATTATGCCATCATAGGAGCCTTGAAG
ACTGAGAAATATAACCTAATATATGAGTACCATAGCCAAATTCAG
GACTGGCATGTTCTCTTACCCATGATTCAAGATCCAGAAACATTC
GAAAAATGTCATGATTTAAGCCTTGCATGTGACTTTATATGCCTT
CTCCAACATGCTATAAAATATAACATGCTTTCTATCCTTGTCAAA
TATAAGGAAGACTTACTAAATGTAAGGATTAGGTATCGTATCCAA
TCCCTGTTTGTTTTGGCATGTGAAAACCGGAGAATTGAGATTATT
GAATGGATAGGTCAAAATCTGCCAATTCCTGAACCTGATGCCATT
TTTAGCATTGCTGTTGCTACAAGAGATTTAGAACTGTTTTCCTTA
GGGTACAAGATTATTTTTGATTACATGCAAAGACAGGGAATCTTT
CAATTAACCAATGGAGTTCGCATGGTTGTGCTAAATCGTCACATT
AGCATGGCAATAGATAATGGTCTTTTACCCTTTGTTCTGGAAACT
TTAAAACATGGTGGGAATATACATAGAGCCTTATCTTATGCAGTA
ACACACAATAAAAGAAAAATTCTGGATTATCTTATTCGCCAGAAA
AATATAGCCCCTAATACAATTGAAAGACTTTTATATCTGGCCGTG
AAAAATCAATCATCCAGAAAAACTTTGAACTTGTTACTATCTTAC
ATAAATTACAAGGTGAAAAATGTTAAAAAGCTGGTAGAGCATGTA
GTAAATGAGAAATCCACTCTTGTGTTAAAAATTTTATTAGAAAAA
AAGGAAAATCTAGTGGATGCTGTTTTAACAAGACTTGTAAAACAT
TCTACATATTTCCAGGTGAGAGAATTTATCCAGGAGTTTTCCATC
AGCCCAGAAAAATTCATTAAAATAGCTGTGCGGGAAAAGAAAAAT
GTGTTGATCGAGGCTATTTCTGAAGATATTTGGGAAAATCCCACA
GAAAGAATTACTTATCTCAAACAGATAGTGCACACCATAAAATAT
GAAAGTGGAAGGCGATTTTTGGTAGACATCATTCACAGCATTTAC
CAAAGTTACTCACTAAAACACGAAGATATTCTTAAACTGGCAACA
TTTTATGTCAAACACAATGCAACCACCCATTTTAAAGATCTCTGC
AAATATCTTTGGCTGAACAGAGGAACAGAAAGTAAAAAACTCTTT
TTAGAGTGTTTAGAAATTGCTGATGAGAAGGAGTTTCCTGATATT
AAAAGCATTGTGAGTGAATATATTAACTACTTGTTTACTGCAGGA
GCTATTACCAAGGAAGAAGCCTATGGATGCTTTAGAGTAGCCATG
TATTAA
Warmbaths MGF 505 6R
SEQ ID No. 264
ATGTTCTCCCTACAGGACCTCTGTCGGAAGAACATTTTCTTCCTT
CCAAACGATTTTAGCAAGCATACCCTACAATGGCTGGGGTTATAT
TGGAAAGAGCATGGATCCGTCCATCGAGCAGAAAAAGACAGCATA
ATGATACAGAATGAATTGGTTCTTTCTATCAATGATGCTTTACTG
CTTGCAGGAGAGGAGGGGGACATAGATGTAGTACAGCTCTTGTTA
TTATGGGAGGGTAATCTGCATTATGCCATCATAGGAGCCTTGAAG
ACTGAGAAATATAACCTAATATGTGAGTACCATAGCCAAATTCAG
GACTGGCATATTCTCCTACCCATGATTCAAGATCCAGAAACATTC
GAAAAATGTCATGATTTAAGCCTTGCATGTGACTTTATATGCCTT
CTCCAACATGCTGTAAAATATAACATGCTTTCTATCCTTGTCAAA
TATAAGGAAGACTTACTAAATGTAAGGATTAGGTATCGTATCCAA
TCCCTGTTTGTTTTGGCATGCGAAAACCGGAGAATTGAGATTATT
CATTGGATAGGCCAAAATCTGCCAATTCCTGAACCTGATGCCATT
TTTAGCATTGCTGTTGCTACAAGAGATTTAGAACTGTTTTCCTTA
GGGTACAAGATTATTTTTGATTACATGCAAAGACAGGGAATCTTT
CAATTATCCAATGGAGTTCGCATGGTTGTGCTAAATCGTCACATT
AGCATGGCAATAGATAATGGACTTTTACCCTTTGTTCTGGAAACT
TTAAAACATGGTGGGAATATACATAGAGCCTTATCTTATGCAGTA
ACACACAATAGAAGAAAAATTCTGGATTATCTTATTCGCCAGAAA
AATATAGCCCCTAATACAATTGAAAGACTTTTATATCTGGCCGTG
AAAAATCAATCTTCCAGGAAAACTTTGAACTTGTTGCTATCTTAC
ATAAATTACAAGGTGAAAAATGTTAAAAAGCTGGTAGAGCATGTA
GTAAATGAGAAATCCACTCTTGTGTTAAAAATTTTATTGGAAAAA
AAGGAAAATCTAGTGGATGCTGTTTTAACAAGACTTGTAAAACAT
TCTACATATTTCCAGGTGAGAGAATTTATCCAGGAGTTTTCCATC
AGCCCAGAAAAATTCATTAAAATAGCTGTGCGGGAAAAGAAAAAT
GTGTTAATCGAGGCTATTTCTGAAGATATTTGGGAAAATCCCACA
GAAAGAATTACTTATCTCAAACAGATAGTGCACACCATAAAATAT
GAAAGTGGAAGACGATTTTTGATAGAGATCATTCACAGCATTTAC
CAAAGTTACTCACTAAAACACGAAGATATTCTTAAACTGGCAACA
TTTTATGTCAAACACAATGCAACCACCCATTTTAAAGATCTCTGC
AAATATCTTTGGCTGAACAGAGGAACAGAAAGTAAGAAACTGTTT
TTAGAGTGTTTAGAAATTGCTGATGAGAAGGAGTTTCCTGATATT
AAAAGTATTGTGAGTGAATATATTAACTACTTGTTTACTGCAGGA
ACTATTACCAAGGAAGAAATCATGCAAGCCTATGATGCTTTAGAG
TAG
Warthog MGF 505 6R
SEQ ID No. 265
ATGTTCTCCCTTCAGGACCTCTGTCGGAAGAACATTTTCTTCCTT
CCAAATGATTTTAGCAAGCATACCCTACAATGGCTGGGGTTATAT
TGGAAAGAGCATGGATCCGTCCATCGAGCAGAAAAAAACAGCATA
GTGATACAGAATGAATTGGTTCTTTCTATCAATGATGCTTTACAG
CTTGCAGGGGAAGAGGGGGACACAGATGTAGTACAGCTCTTGTTA
TTATGGGAGGGAAATCTGCATTATGCCATCATAGGAGCCTTGAAG
ACTGAGAAATATAATCTAATATGTGAGTACCATAGCCAAATTCAG
GACTGGCATGTTCTCCTCCCCTTGATTCAAGATCCAGAAACATTC
GAAAAATGTCATGATTTAAGCCTTGCATGTGACTTTATATGCCTT
CTACAACATGCTGTAAAATATAACATGCTTTCTATCCTTGTCAAA
TATAAGGAAGACTTACTAAATGTAAGGATTAGGTATCGTATCCAA
TCCCTGTTTGTTTTGGCATGTGAAAACCGGAGAATTGAGATTATT
GATTGGATAGGCCAAAATCTGCCAATTCCTGAACCTGATGCCATT
TTTAGCATTGCTGTTGCTACAAGAGATTTAGAACTGTTTTCCTTA
GGGTACAAGATTATTTTTGATTACATGCAAAGACAGGGAATCTTT
CAATTATCCAATGGAGTTCGCATGGTTGTGCTAAATCGTCACATT
AGCATGGCAATAGATAATGGACTTTTACCCTTTGTTCTGGAAACT
TTAAAACATGGTGGGAATATACATAGAGCCTTATCTTATGCAGTA
ACACACAATAGAAGAAAAATTCTGGATTATCTTATTCGCCAGAAA
AATATAGCCCCTAATACAATTGAAAGACTTTTATATCTGGCCGTG
AAAAATCAATCTTCCAGGAAAACTTTGAACTTGTTGCTATCTTAC
ATAAATTACAAGGTGAAAAATGTTAAAAAGCTAGTAGAGCATGTA
GTAAATGAGAAATCCACTCTTGTGTTAAAAATTTTATTGGAAAAA
AAGGAAAATCTAGTGGATGCTGTTTTAACAAGACTTGTAAAACAT
TCTACATATTTCCAGGTGAGAGAATTTATCCAGGAGTTTTCCATC
AGCCCAGAAAAATTCATTAAAATAGCTGTGCGGGAAAAGAAAAAT
GTGTTAATCGAGGCTATTTCTGAAGATATTTGGGAAAATCCCACA
GAAAGAATTACTTATCTCAAACAGATAGTGCACACCATAAAATAT
GAAAGTGGAAGACGATTTTTGATAGACATCATTCACAGCATTTAC
CAAAGTTACTCACTAAAACACGAAGATATTCTTAAACTGGCAACA
TTTTATGTCAAATACAATGCAATCACCCATTTTAAAGATCTCTGC
AAATATCTTTGGCTGAACAGAGGAACAGAAAGTAAGAAACTGTTT
TTAGAGTGTTTAGAAATTGCTGATGAGAAGGAGTTTCCTGATATT
AAAAGCATTGTGAGTGAATATATTAACTACTTGTTTACTGCAGGA
GCTATTACCAAGGAAGAAATCATGCAAGCCTATGATGCTTTAGAG
TAG

In an embodiment the invention provides an ASFV which comprises one or more, such as all, of the following sequences from the Benin 97/1 strain:

### SEQ ID No. 55 (MGF 360 6L), SEQ ID No. 68 (MGF 360 10L), SEQ ID No. 78 (MGF 360 50 11L), SEQ ID No. 96 (MGF 360 12L), SEQ ID No. 108 (MGF 360 13L) and SEQ ID No. 120 (MGF 360 14L), and

### SEQ ID No. 141 (MGF 505 1R) and SEQ ID No. 151 (MGF 505 2R).

In an embodiment the invention provides an ASFV which comprises one or more, such as all, of the following sequences from the China/2018/AnhuiXCGQ strain:

### SEQ ID No. 52 (MGF 360 6L), SEQ ID No. 63 (MGF 360 10L), SEQ ID No. 76 (MGF 360 11L), SEQ ID No. 89 (MGF 360 12L), SEQ ID No. 101 (MGF 360 13L), SEQ ID No. 113 (MGF 360 14L) and SEQ ID No. 125 (MGF 360 21R), and

### SEQ ID No. 135 (MGF 505 1R) and SEQ ID No. 148 (MGF 505 2R).

In an embodiment the invention provides an ASFV which comprises one or more, such as all, of the following sequences from the Georgia 2007/1 strain:

### SEQ ID No. 51 (MGF 360 6L), SEQ ID No. 62 (MGF 360 10L), SEQ ID No. 75 (MGF 360 11L), SEQ ID No. 88 (MGF 360 12L), SEQ ID No. 100 (MGF 360 13L), SEQ ID No. 112 (MGF 360 14L) and SEQ ID No. 124 (MGF 360 21R), and

### SEQ ID No. 134 (MGF 505 1R) and SEQ ID No. 147 (MGF 505 2R).

In an embodiment the invention provides an ASFV which comprises one or more, such as all, of the following sequences from the Ken05/Tk1 strain:

### SEQ ID No. 35 (MGF 110 6L), SEQ ID No. 44 (MGF 110 8L), and

### SEQ ID No. 58 (MGF 360 6L), SEQ ID No. 65 (MGF 360 10L), SEQ ID No. 86 (MGF 360 11L) and SEQ ID No. 130 (MGF 360 21R), and

### SEQ ID No. 145 (MGF 505 1R) and SEQ ID No. 156 (MGF 505 2R).

In an embodiment the invention provides an ASFV which comprises one or more, such as all, of the following sequences from the Ken06.Bus strain:

### SEQ ID No. 36 (MGF 110 6L), and

### SEQ ID No. 61 (MGF 360 6L), SEQ ID No. 64 (MGF 360 10L), SEQ ID No. 87 (MGF 360 11L), SEQ ID No. 99 (MGF 360 12L), SEQ ID No. 111 (MGF 360 13L), SEQ ID No. 123 (MGF 360 14L) and SEQ ID No. 132 (MGF 360 21R), and

### SEQ ID No. 144 (MGF 505 1R) and SEQ ID No. 158 (MGF 505 2R).

In an embodiment the invention provides an ASFV which comprises one or more, such as all, of the following sequences from the Kenya 1950 strain:

### SEQ ID No. 37 (MGF 110 6L), SEQ ID No. 45 (MGF 110 8L), and

### SEQ ID No. 60 (MGF 360 6L), SEQ ID No. 66 (MGF 360 10L), SEQ ID No. 85 (MGF 360 11L), SEQ ID No. 98 (MGF 360 12L), SEQ ID No. 110 (MGF 360 13L), SEQ ID No. 122 (MGF 360 14L) and SEQ ID No. 131 (MGF 360 21R), and

### SEQ ID No. 143 (MGF 505 1R) and SEQ ID No. 157 (MGF 505 2R).

In an embodiment the invention provides an ASFV which comprises one or more, such as all, of the following sequences from the L60 strain:

### SEQ ID No. 54 (MGF 360 6L), SEQ ID No. 67 (MGF 360 10L), SEQ ID No. 77 (MGF 360 11L), SEQ ID No. 95 (MGF 360 12L), SEQ ID No. 107 (MGF 360 13L) and SEQ ID No. 119 (MGF 360 14L), and

### SEQ ID No. 140 (MGF 505 1R) and SEQ ID No. 150 (MGF 505 2R).

In an embodiment the invention provides an ASFV which comprises one or more, such as all, of the following sequences from the Malawi Lil-20/1 (1983) strain:

### SEQ ID No. 38 (MGF 110 6L), SEQ ID No. 46 (MGF 110 8L), and

### SEQ ID No. 59 (MGF 360 6L), SEQ ID No. 74 (MGF 360 10L), SEQ ID No. 84 (MGF 360 11L), SEQ ID No. 97 (MGF 360 12L), SEQ ID No. 109 (MGF 360 13L), SEQ ID No. 121 (MGF 360 14L) and SEQ ID No. 133 (MGF 360 21R), and

### SEQ ID No. 146 (MGF 505 1R) and SEQ ID No. 155 (MGF 505 2R).

In an embodiment the invention provides an ASFV which comprises one or more, such as all, of the following sequences from the Mkuzi 1979 strain:

### SEQ ID No. 39 (MGF 110 6L), SEQ ID No. 47 (MGF 110 8L), and

### SEQ ID No. 57 (MGF 360 6L), SEQ ID No. 69 (MGF 360 10L), SEQ ID No. 79 (MGF 360 11L), SEQ ID No. 94 (MGF 360 12L), SEQ ID No. 106 (MGF 360 13L), SEQ ID No. 118 (MGF 360 14L) and SEQ ID No. 128 (MGF 360 21R), and

### SEQ ID No. 138 (MGF 505 1R) and SEQ ID No. 149 (MGF 505 2R).

In an embodiment the invention provides an ASFV which comprises one or more, such as all, of the following sequences from the Pretorisuskop/96/4 strain:

### SEQ ID No. 40 (MGF 110 6L), SEQ ID No. 48 (MGF 110 8L), and

### SEQ ID No. 56 (MGF 360 6L), SEQ ID No. 70 (MGF 360 10L), SEQ ID No. 81 (MGF 360 11L), SEQ ID No. 93 (MGF 360 12L), SEQ ID No. 105 (MGF 360 13L), SEQ ID No. 117 (MGF 360 14L) and SEQ ID No. 127 (MGF 360 21R), and

### SEQ ID No. 142 (MGF 505 1R) and SEQ ID No. 153 (MGF 505 2R).

In an embodiment the invention provides an ASFV which comprises one or more, such as all, of the following sequences from the Tengani 62 strain:

### SEQ ID No. 41 (MGF 110 6L), and

### SEQ ID No. 71 (MGF 360 10L), SEQ ID No. 82 (MGF 360 11L), SEQ ID No. 90 (MGF 360 12L), SEQ ID No. 102 (MGF 360 13L), SEQ ID No. 114 (MGF 360 14L) and SEQ ID No. 126 (MGF 360 21R), and

### SEQ ID No. 136 (MGF 505 1R) and SEQ ID No. 152 (MGF 505 2R).

In an embodiment the invention provides an ASFV which comprises one or more, such as all, of the following sequences from the Warmbaths strain:

### SEQ ID No. 42 (MGF 110 6L), SEQ ID No. 49 (MGF 110 8L), and

### SEQ ID No. 53 (MGF 360 6L), SEQ ID No. 73 (MGF 360 10L), SEQ ID No. 80 (MGF 360 11L), SEQ ID No. 92 (MGF 360 12L), SEQ ID No. 104 (MGF 360 13L), SEQ ID No. 116 (MGF 360 14L) and SEQ ID No. 129 (MGF 360 21R), and

### SEQ ID No. 137 (MGF 505 1R) and SEQ ID No. 154 (MGF 505 2R).

In an embodiment the invention provides an ASFV which comprises one or more, such as all, of the following sequences from the Warthog strain:

### SEQ ID No. 43 (MGF 110 6L), SEQ ID No. 50 (MGF 110 8L), and

### SEQ ID No. 72 (MGF 360 10L), SEQ ID No. 83 (MGF 360 11L), SEQ ID No. 91 (MGF 360 12L), SEQ ID No. 103 (MGF 360 13L), SEQ ID No. 115 (MGF 360 14L), and

### SEQ ID No. 139 (MGF 505 1R).

The translation products (i.e. protein sequences) of these genes are given below:

translation of Benin 97/1 MGF 110-11L (A9JKR9)
SEQ ID No. 1
MKYSWKNGGGDYWPIIIRHCCFYLVFSIAFVGYIVFAYYKNLHLN
TAMKLLALLCILIWLSQPGLNRPLSIFYMKQNLPRTYTPPIRELE
YWCTYGKHCDFCWECRNGICKNKVWDDMSSVQEHSYPMEHCMIHR
QCKYIRDGPIFQVECTMQTSDATHLINA
translation of Benin 97/1 MGF 110-12L (A9JKS1)
SEQ ID No. 2
MKVFLGLLLGYSTILILTYQSPTTQWCFYEISLKILNHHSMEKWR
DKNWSIIIRYYCFYLVFSFAFAGCVAFAICKNLRLCTTMKLLMLL
NILVLLSQPILNN
translation of Benin 97/1 MGF 360-6L (A9JKS8)
SEQ ID No. 3
MNSLQVLTKKVLIENKAFSEYHEDDIFILQQLGLWWHNGPIGFCK
QCKMVTSGSMSCSDVDSYELDRALVRAVKKNQTDLIKLFVLWGAN
INYGIICAKTERTKDLCIELGANPEFLDVGLYNMFVDLIKQQKVL
LAIDIYYDNISILDSFDSHDFYVLIDFIYNCFILNLDEKEKMIKN
TYVLKFWFKIAIEFNLIKPIRFLSKKFPHLDYWRLKTAVYLGNVD
EIHHAYFQENIRLDPNDMMSLACMYPQNKLGIYYCFALGANINTA
LETLIGFINHEVNREITFFSNYGIWSNVHFCISLGANPYTKKIQE
TLLRQEKNVIMKLLFKKGLLSPHSILHKKILEPSEVRKIISTYEY
TETFHSFSLLRDNLR
translation of Benin 97/1 MGF 360-10L
SEQ ID No. 4
MVPSLQSFAKKVLASQHVSIDYHVILERCGLWWYKAPISLDCKHM
LIKLPNFADGLDLNTALMLATKENNYQLIKMFTDWGADINYGLIC
ANTPPIREFCWELGAKYQVDKKKIMHIFFKLIHPNTTSNNIILCL
KFFNDNPFSAYVIIREIKSCIHWKLKNLAEDTNVLSNISDGDMLT
IYCFIVALQDNLREAISYVYQHFKYLNTWWLTCALCYNKLFDLHN
LYEKEKIRMDMDEMMRIACTKDNNFLTIYYCFILGANINLAMIAS
IRFYNMDNLFFCIDLGADAFEEAKALAEQQNYYLISHRLSLDIYS
PDSSLLTLKEADPNKIYRLLKNYKSKSMLAYLNYDINDTSL
translation of Benin 97/1 MGF 360-11L (A9JKU4)
SEQ ID No. 5
MLPSLQSLTKKVLAGQCVSVDHYHILKCCGLWWHNGPIMLHIRRN
KLFIRSTCFSQGIELNIGLMKAVKENNHDLIKLFTEWGADINYGM
ICALTENTRDLCKELGAKEYLEREYILKIFFDTTRDKTSSNIIFC
HEVFSNNPNLRIIDNLDLRGEIMWELRGLMEITFMLDHDDSFSTV
LTKYWYAIAVDYDLKDAIRYFYQKYPRLHRWRLMCALFYNNVFDL
HELYEIERVRMDIDEMMHIACIQDYSYSAIYYCFIMGANINQAML
VSIQNYNLGNLFFCIDLGANAFEEGKALAEQKENYLIAHALSLKH
YNPVISLLSNVMDPEKINYMLKNYHSINMGIFLDYEQR
translation of Benin 97/1 MGF 360-12L (A9JKU9)
SEQ ID No. 6
MLPSLQSLTKKVLAGQCVPTNQHYLLKYYDLWWYNAPITFDHNLR
LIKSSGIKEGLDLNTALVKAVRENNYSLIKLETEWGADINYGLVS
VNTEHTRDLCQELGAKEILNEEEILQIFIDLKFHKTSSNIILCHE
VFSNNPILQKVNNLKLRIEIFWELRELIEKTDLLNNEFLLSTLLL
KYWYAIAVRYSLKEAIQYFYQKYTHMNTWRLTCALCFNNVFDLHE
AYEKDKIHMDIEEMMRIACIKDHNLSTMYYCYMLGANINQAMLTS
IQYYNIENMFFCMDLGADVFEEGTTALGEGYELIKNILSLKIYSP
TTIPLPKSTDPEIIDHALKNYFSKNMMIFLSYDLR
translation of Benin 97/1 MGF 360-13L (A9JKU7)
SEQ ID No. 7
MSLPLSLQTLVKKTVASQCLSIDEHCILKYCGLWWHDAPLKLCMD
RGRIQIKSGFLGEDIDLRVALIIAVKENNYSLIKLFTEWGANINY
SLLSINTKHIRELCRQLGAKETLEDNDIFRIFTRIMHNKTSGSII
LCHEIFMNNPMLENKFVIQLRGLIYKRLWGLIEIKETDELNDLLV
KYWYAKAVQYVCKNAICFLDEKYTDLNEWRLKCLLYYNKIYELHE
MYHKKKVQIDVHDMICLACAKDNNLLTIYYCYALGGNINQAMLTS
VQYYNVGNIFFCIDLGGNAFEEGRAIAEQKGYNFLSHSLTLDIYS
SDASLPLNLKDPEKISSLLKDYKSKNLSIIWEYSHNIL
translation of Benin 97/1 MGF 360-14L (A9JKV1)
SEQ ID No. 8
MLSLQTLAKKVVACNYLSSDYDYTLQRFGLWWDLGPIHLCNNCKQ
VFSYKHLQCFSEDDLCLEAALVKAVKSDNLELIRLFVDWGANPEY
GLIRVPAVYLKRLCAELGGLTPVSEPRLLEILKEVANLKSCAGVL
LGYDMFCHNPLLETVTRTTLDTVTYTCSNIPLTGDTAHLLLTKFW
FALALRHNFTKAIHYFYKRHKNQLYWRVACSLYFNNIFDIHELCR
EKEICISPNLMMKFACLREKNYAAIYYCHRLGASLDYGMNLSIYN
NNTLNMFFCIDLGAADFDRAQLIAHKAYMYNLSNIFLVKQLFSRD
VTLVLDVTEPQEIYDMLKTYTSKNMKRAEEYLTAHPEIIVID
translation of Benin 97/1 MGF 505-1R (A9JKU5)
SEQ ID No. 9
MFSLQNLCRKTLPDCKLPEFFDDYILQLLGLYWENHGTIQRAGNN
CVLIQQHTLIPVNEALRIAASEENYEIVGLLLAWEGNLYYAIIGA
LEGNRYNLIRKYDDQIKDHHDILPFIDDPIIFHKCHIMRRCFFDC
ILYQAVKYSKFRVLLYFKYTLEDDLPLVHLLIEKACEDHNYEVIK
WIYENLHVCHIIDTFDCAIAHKDLRLYCLGYTFIYNRIVPYKYHH
LDILILSSLQLLHKVAAKGYLDFILETLKYDHNIDNLDVILTQAA
TYNHRKILTYFIPQSTYAQIEQCLFVAIKTKSSKKTLNLLLSHLN
LSIKLIQKISQYVATFNSTNIIGILSMKRKKKIYLDIILTKFVKN
AIFNKFVVRCMERFSINPERIVKMAARINKMMLVKKISEHVWKNH
AARLKHLKHAVHTMKHKDGKNRLMNFIYEHCYYHMQGEEIFSLAR
FYAIHHAPKLFDVFYNCCILDTIRFKSLLLDCSHIIGKNAHDATN
INIVNKYIGNLFAMGVLSKKEILQDYPSIYSKHYMP
translation of Benin 97/1 MGF 505-2R (A9JKV3)
SEQ ID No. 10
MFSLQDLCRKHLFILPDVFGEHVLQRLGLYWRCHGSLQRIGDDHI
LIRRDLILSTNEALRMAGEEGNNEVVKLLLLWKGNLHYAVIGALQ
GDQYDLIHKYENQIGDFHFILPLIQDANTFEKCHALERFCGVSCL
LKHATKYNMLPILQKYQEELSMRAYLHETLFELACLWQRYDVLKW
IEQTMHVYDLKIMFNIAISKRDLTMYSLGYIFLFDRGNTEATLLT
QHLEKTAAKGLLHFVLETLKYGGNIDTVLTQAVKYNHRKLLDYFL
RQLPRKHIEKLLLLAVQEKASKKTLNLLLSHLNYSVKRIKKLLRY
VIEYESTLVIKILLKKRVNLIDAMLEKMVRYFSATKVRTIMDELS
ISPERVIKMAIQKMRTDIVIHTSYVWEDDLERLTRLKNMVYTIKY
EHGKKMLIKVMHGIYKNLLYGEREKVMFHLAKLYVAQNAATQFRD
ICKDCYKLDVARFKPRFKQLILDCLEIVTKKSCYSILEILEKHII
SLFTMKVMTEEEKNLCLEILYKVIHYKTIQC
MGF 110 5L protein sequences
Ken05/Tk1 MGF 110 5L protein
SEQ ID No. 306
MLVIFLGLLGLLANQVLGLPNQPAGQLHPTDNPPKEELGYWCTYT
ESCKFCWNCQNGLCEGKLENTTILENEYVQSCIVSRWLNKCMYDL
GQGIDHVMACSEPKPWNPYKILKREWKKNVSQN
Ken06.Bus MGF 110 5L protein
SEQ ID No. 307
MLVIFLGLLGLLANQVLGLPNQPAGQLHPTDNPPKEELGYWCTYT
ESCKFCWNCQNGLCEGKLENTTILENEYVQSCIVSRWLNKCMYDL
GQGIHHVMPCSEPKPWNPYKILKKEWKENVSQN
Malawi Lil-20/1/1983 MGF 110 5L protein
SEQ ID No. 308
MLVIFLGILGLLANQVSSQLVGQLHPTENPSENELEYWCTYMECC
QFCWDCQDGLCVNKLGNTTILENEYVHPCIVSRWLNKCMYDLGQG
IDHVMVCSQPKYWNPYKILKKEWKENNSQN
Mkuzi 1979 MGF 110 5L protein
SEQ ID No. 309
MLVIFLGILGLLANQVSSQLVGQLHPTENPSENELEYWCTYMECC
QFCWDCQDGLCVNKLGNTTILENEYVHPCIVSRWLNKCMYDLGQG
IDHVMVCSQPKYWNPYKILKKEWKENNSQN
Pretorisuskop/96/4 MGF 110 5L protein
SEQ ID No. 310
MLVIFLGILGLLANQVSSQLVGQLHPTENPSENELEYWCTYMECC
QFCWDCQNGLCVNKLGNTTILENEYVHPCIVSRWLNKCMYDLGQG
IDHVMVCSQPKYWNPYKILKKEWKENNSQNK
Tengani62 MGF 110 5L protein
SEQ ID No. 311
MLVIFLGILGLLVNQVSSQPVGQLHPTENPSENELEYWCTYMECC
QFCWDCQNGLCVNKLGNTTILENEYVHPCIVSRWLNKCMYDLGQG
IDHVMVCSQPKYWNPYKILKKEWKENNS
Warmbaths MGF 110 5L protein
SEQ ID No. 312
MLVIFLGILGLLANQVSSQLVGQLHPTENPSENELEYWCTYMECC
QFCWDCQNGLCVNKLGNTTILENEYVHPCIVSRWLNKCMYDLGQG
IDHVMVCSQPKYWNPYKILKKEWKENNSQNK
Warthog MGF 110 5L protein
SEQ ID No. 313
MLVIFLGILGLLANQVSSQLVGQLHPTENPSENELEYWCTYMECC
QFCWDCQNGLCVNKLGNTTILENEYVHPCIVSRWLNKCMYDLGQG
IDHVMVCSQPKYWNPYKILKKEWKENNSQNK
China/2018/AnhuiXCGQ MGF 110 5L/6L fusion protein
SEQ ID No. 314
MLVIFLGILGLLASQVSSQLVGQLRPTEEPPEEELEYWCAYMESC
QFCWDCQDGTCINKIDGSVIYKNEYVKSCLVSRWLDKCMYDLDKG
IYHTMNCNQVLGLPNQPAGQLHPTDNPPQEELEYWCTYTENCKFC
WNCQNGLCEGKLENTTILENEYVQSCIVSRWLNKCMYDLGQGIHH
VMACSEPKPWNPYKILKREWKENNS
Georgia 2007/1 MGF110 5L/6L fusion protein
SEQ ID No. 315
MLVIFLGILGLLASQVSSQLVGQLRPTEEPPEEELEYWCAYMESC
QFCWDCQDGTCINKIDGSVIYKNEYVKSCLVSRWLDKCMYDLDKG
IYHTMNCNQVLGLPNQPAGQLHPTDNPPQEELEYWCTYTENCKFC
WNCQNGLCEGKLENTTILENEYVQSCIVSRWLNKCMYDLGQGIHH
VMACSEPKPWNPYKILKREWKENNS
MGF 110 12L protein sequences
Benin 97/1 MGF 110 12L protein
SEQ ID No. 316
MKVFLGLLLGYSTILILTYQSPTTQWCFYEISLKILNHHSMEKWR
DKNWSIIIRYYCFYLVFSFAFAGCVAFAICKNLRLCTTMKLLMLL
NILVLLSQPILNN
China/2018/AnhuiXCGQ MGF 110 12L protein
SEQ ID No. 317
MKVFLGLLLGYSTILILTYQSPTTPWCFYEISLKIPNHHSMKCCS
YPRLYEHEMFMEKWRDKNWPIIIRYYCFYLVFSFVFAGCVAFAIC
KNLRLSTTMKLLMLLSILVLLLSQPILNN
Georgia 2007/1 MGF 110 12L protein
SEQ ID No. 318
MKVFLGLLLGYSTILILTYQSPTTPWCFYEISLKIPNHHSMKCCS
YPRLYEHEMFMEKWRDKNWPIIIRYYCFYLVFSFVFAGCVAFAIC
KNLRLSTTMKLLMLLSILVLLLSQPILNN
Ken05/Tk1 MGF 110 12L protein
SEQ ID No. 319
MKVFLGLLLGFSIILILTYQSPTTQHPPKEELAYWCTYAKSCDFC
WDCQNDTCINKVINESISITSIVNCRVTRDSQSCFYEISVKIPNH
HSMECSYPRLYEHEMFMEKWRDEYWPIIIKQCCFYLVFSIAFAGC
VAFAICKNLRLRTTIKLLILLSILVWLSQPVLN
Ken06.Bus MGF 110 12L protein
SEQ ID No. 320
MKVFLALLLGYLTILILTYQTPTTQHPPKEELPYWCTYVKNCDLC
WDCQDSIYWNKVISESISINSIINCRVTCDSQSQSCEYEILLKIP
NHHSMECSYPGSYENEMFMEKWRDENWSIIIKHYCFYLVFSFAFA
GCVAFAICKNLRLSTTMKLLMLLSILVCLSQPILNN
Kenya 1950 MGF 110 12L protein
SEQ ID No. 321
MKVFLGLLLGFSIILILTYQSPTTQHPPKEELAYWCTYAKSCDFC
WDCQNDTCINKVINESISITSIVNCRVTRDSQSCFYDISVKIPNH
HSMECSYPRLYEHEMFMEKWRDEYWPIIIKQCCFYLVFSFAFAGC
VAFAICKNLRLRTTIKLLILLSILVWLSQPILNN
L60 MGF 110 12L protein
SEQ ID No. 322
MKVFLGLLLGYSTILILTYQSPTTQWCFYEISLKILNHHSMEKWR
DKNWSIIIRYYCFYLVFSFAFAGCVAFAICKNLRLCTTMKLLMLL
NILVLLSQPILNN
Malawi Lil-20/1/1983 MGF 110 12L protein
SEQ ID No. 323
MKVFLGLLLGYSTILILTYQSPTTQHPPKEELEYWCTYAKTCDFC
WDCQNDTCINKVINESISMNSIVNCRVTRDSQSQSCFYEISLKIP
NYHSMECSYPRLYKHFMSMEKWRDENWPILIRHYCFYLVFSFAFA
GCVAFAICKNLRLRTTMKLLMLLSILVLLSQPILNN
Mkuzi 1979 MGF 110 12L protein
SEQ ID No. 324
MNATMKVFLGLLLGYSTILILTYQSPTTQWCFYEISLKILNHHSM
EKWRDKNWSIIIRYYCFYLVFSFAFAGCVAFAICKNLRLCTTMKL
LMLLGILVLLSQPILNN
Warmbaths MGF 110 12L protein
SEQ ID No. 325
MKVFLGLLLGYSTILILTYQSPATQWCFYEISLKIPNHHSMECSY
PRLYKHFIFMEKWRDKNWSIIIRYYCFYLVFSFAFAGCIAFAICK
NLRLCTTMKLLMLLSILVLLSQPILNN
Warthog MGF 110 12L protein
SEQ ID No. 326
MKVFLGLLLGYSTILILTYQSPATQWCFYEISLKIPNHHSMECSY
PRLYKHFMFMKKWRDKNWSIIIRYYCFYLVFSFAFAGCIAFAICK
NLRLCTTMKLLMLLSILVLLSQPILNN
MGF 360 12L protein sequences
Georgia 2007/1 MGF 360 12L protein
SEQ ID No. 159
MLPSLQSLTKKVLAGQCVPTNQHYLLKCYDLWWHDAPITFDHNLR
LIKSAGIKEGLNLNTALVKAVRENNYNLIKLFAEWGADINYGLVS
VNTEHTWDLCRELGAKETLNEEEILQIFIDLKFHKTSSNIILCHE
VFSNNPILQKVNNIKMRIEIFWELRELIVKTDLLNNEFSLSTLLL
KYWYAIAIRYNLKEAIQYFYQKYTHLNTWRLTCALCFNNVFDLHE
AYEKDKIHMDIEEMMRIACIKDHNLSTMYYCYVLGANINQAMLSS
IQYYNIENMFFCIDLGADVFEEGTTALGEGYELIKNILSLKIYSP
ATTPLPKSTDPEIIDHALKNYVSKNMMIFLTYDLR*
China/2018/AnhuiXCGQ MGF 360 12L protein
SEQ ID No. 160
MLPSLQSLTKKVLAGQCVPTNQHYLLKCYDLWWHDAPITFDHNLR
LIKSAGIKEGLNLNTALVKAVRENNYNLIKLFAEWGADINYGLVS
VNTEHTWDLCRELGAKETLNEEEILQIFIDLKFHKTSSNIILCHE
VFSNNPILQKVNNIKMRIEIFWELRELIVKTDLLNNEFSLSTLLL
KYWYAIAIRYNLKEAIQYFYQKYTHLNTWRLTCALCFNNVFDLHE
AYEKDKIHMDIEEMMRIACIKDHNLSTMYYCYVLGANINQAMLSS
IQYYNIENMFFCIDLGADVFEEGTTALGEGYELIKNILSLKIYSP
ATTPLPKSTDPEIIDHALKNYVSKNMMIFLTYDLR*
Tengani 62 MGF 360 12L protein
SEQ ID No. 161
MLPSLQSLTKKVLAGQCVPTNQHYLLKCYDLWWHDAPITFDHNLR
LIKSAGIKEGLNLNTALVKAVRENNYNLIKLFAEWGADINYGLVS
VNTEHTWDLCRELGAKETLNEEEILQIFIDLKFHKTSSNIILCHE
VFSNNPILQKVNNIKMRIEIFWELRELIEKTDLLNNEFSLSTLLL
KYWYAIAIRYNLKEAIQYFYQKYTHLNTWRLTCALCFNNVFDLHE
AYEKDKIHMDIEEMMRIACIKDHNLSTMYYCYVLGANINQAMLSS
IQYYNIENMFFCIDLGADVFEEGTTALGEGYELIKNILSLKIYSP
ATTPLPKSMDPEIIDHALKNYVSKNMMIFLTYDLR*
Warthog MGF 360 12L protein
SEQ ID No. 162
MLPSLQSLTKKVLAGQCVPTNQHYLLKCYDLWWHDGPITFDHNLK
LIKSAGIKEGLDLNTALVKAVRENNYNLIKLFAEWGANINYGLVS
VNTEHTRDLCRELGAKETLNEEEILQIFIDLKFHKTSSNIILCHE
VFSNNPILQKVNNIKMRIEIFWELRELIEKTDLLNNEFSLSALLL
KYWYAIAIRYNLKEAIQYFYQKYTHLNTWRLTCALCFNNVFDLHE
AYEKDKIHMDIEEMMRIACIKDHNLSTMYYCYVLGANINQAMLTS
IQYYNIENMFFCMDLGADAFEEGTIALGEGYKLIKNILSLKIYSP
ATTPLPKSTDPEIIDHALKNYVSKNMMIFLTYDLR*
Warmbaths MGF 360 12L protein
SEQ ID No. 163
MLPSLQSLTKKVLAGQCVPTNQHYLLKCYDLWWHDGPITFDHNLK
LIKSAGIKEGLDLNTALVKAVRENNYNLIKLFAEWGADINYGLVS
VNTEHTRDLCRELGAKETLNEEEILQIFIDLKFHKTSSNIILCHE
VFSNNPILQKVNNIKMRIEIFWELRELIEKTDLLNNEFSLSTLLL
KYWYAIAIRYNLKEAIQYFYQKYTHLNTWRLTCALCFNNVFDLHE
AYEKDKIYMDIEEMMRIACIKDHNLSTMYYCYVLGANINQAMLTS
IQYYNVENMFFCMDLGADAFEEGTIALGEGYKLIKNILSLKIYSP
ATTPLPKSTDPEIIDHAVKNYVSKNMMIFLTYDLR*
Pretorisuskop/96/4 MGF 360 12L protein
SEQ ID No. 164
MLPSLQSLTKKVLAGQCVPTNQHYLLKCYDLWWHDGPITFDHNLK
LIKSAGIKEGLDLNTALVKAVRENNYNLIKLFAEWGANINYGLVS
VNTEHTWDLCRELGAKETLNEEEILQIFIDLKFHKTSSNIILCHE
VFSNNPILQKVNNIKMRIEIFWELRELIEKTDLLNNEFSLSTLLL
KYWYAIAIRYNLKEAIQYFYQKYTHLNTWRLTCALCFNNVFDLHE
AYEKDKIHMDIEEMMRIACIKDHNLSTMYYCYVLGGNINQAMLTS
IQYYNIENMFFCMDLGADAFEEGTIALGEGYKLIKNILSLKIYSP
ATTPLPKSTDPEIIDHALKNYVSKNMMIFLTYDLR*
Mkuzi 1979 MGF 360 12L protein
SEQ ID No. 165
MLPSLQSLTKKVLAGQCVPTNQHYLLKYYDLWWHDAPITFDHNLR
LIKSAGIKEGLDLNTALVKAVKENNYNLIKLFAEWGADINYGLVS
VSSEHTWDLCRELGAKETLNEKEILQIFIDLKFHKTSSNIILCHE
VFSNNPILQKVNNIKMRIEIFWELRELIEKTDLLNNEFSLSTLLL
KYWYAIAIRYNLKEAIQYFYQKYTHLNTWRLTCALCFNNVFDLHE
AYEKDKIYMDIEEMMRIACIKDHNLSTMYYCYMLGANINQAMLTS
IQYYNIENMFFCMDLGADVFEEGTTALGEGYELIKNILSLKIYSP
TTIPLPKSTDPEIIDHALKNYFSKNMMIFLSYDLR*
L60 MGF 360 12L protein
SEQ ID No. 166
MLPSLQSLTKKVLAGQCVPTNQHYLLKYYDLWWYNAPITFDHNLR
LIKSSGIKEGLDLNTALVKAVRENNYSLIKLFTEWGADINYGLVS
VNTEHTRDLCQELGAKEILNEEEILQIFIDLKFHKTSSNIILCHE
VFSNNPILQKVNNLKLRIEIFWELRELIEKTDLLNNEFLLSTLLL
KYWYAIAVRYSLKEAIQYFYQKYTHLNTWRLTCALCFNNVFDLHE
AYEKDKIHMDIEEMMRIACIKDHNLSTMYYCYMLGANINQAMLTS
IQYYNIENMFFCMDLGADVFEEGTTALGEGYELIKNILSLKIYSP
TTIPLPKSTDPEIIDHALKNYFSKNMMIFLSYDLR*
Benin 97/1 MGF 360 12L protein
SEQ ID No. 6
MLPSLQSLTKKVLAGQCVPTNQHYLLKYYDLWWYNAPITFDHNLR
LIKSSGIKEGLDLNTALVKAVRENNYSLIKLFTEWGADINYGLVS
VNTEHTRDLCQELGAKEILNEEEILQIFIDLKFHKTSSNIILCHE
VFSNNPILQKVNNLKLRIEIFWELRELIEKTDLLNNEFLLSTLLL
KYWYAIAVRYSLKEAIQYFYQKYTHMNTWRLTCALCFNNVFDLHE
AYEKDKIHMDIEEMMRIACIKDHNLSTMYYCYMLGANINQAMLTS
IQYYNIENMFFCMDLGADVFEEGTTALGEGYELIKNILSLKIYSP
TTIPLPKSTDPEIIDHALKNYFSKNMMIFLSYDLR*
Malawi Lil-20/1 MGF 360 12L protein
SEQ ID No. 167
MLPSLQSLTKKVLAGQCLPTDQYYLLKCYDLWWYDSPITFDHNLG
LIKSAGIKDGLDLNTALVKAVRENNYNLIKLFTEWGADINYGLVS
VNTEHTRDLCRELGAKETLNEEEILRIFIDLKFYKTSSNIILCHE
VFSNNPLLQKVNNLKMRIEIFWELRELIKKTDLLNNEFSLNTLLL
KYWYAIAVRYNLKEAIQYFYQKYTHLNTWRLTCALCFNNVFDLHE
AYEKDKIYMDLEEMMRVACIKDHNLSTIYYCYVLGANINQAMLAS
IQYYNIENMFFCMDLGADVFEENMPVGEGYELIRNILSLKIYSPS
TAPLPKNTDPEIIDHVLKNYKSKNMMTFLSYDLR*
Kenya 1950 MGF 360 12L protein
SEQ ID No. 168
MLPSLQSLTKKVLAGQCLPEDQHYLLKCYDLWWNNAPITFDHNLR
LIKSAGLQEGLDLNMALVKAVKENNYSLIKLETEWGANINYGLIS
VNTEHTWDLCRELGAKKTLNEGDILQIFIDLKFHKTSSNIILCHE
VFSDNLLLKKVNNLKMRIEIFWELREIIEKTDLLNNEFSLNTLLL
KYWYAIAVRYNLKEAIQYFYQKYTHLNTWRLTCALCFNNVFDLHE
AYEKDKICMDLEEMMRIACIKDHSLSTIYYCYMLGANINQAMLTS
IQYYNIENIFFCMDLGADAFEEGMALVGQEGYEPIRNILSLKIYS
PATTPLPKSTDPEIIDHELKNYFSKNMMVFLTYDLR*
Ken06.Bus MGF 360 12L protein
SEQ ID No. 169
MLPSLQSLTKKVLARQCLPEDQHYLLKCYDLWWNNAPITFDHNLR
LIKLAGIQEGLDLNMALVKAVKENNYSLIKLFTEWGANINYGLIS
VNTEHTWDLCRELGAKKTLNEGDILQIFIDLKFYKTSSNIILCHE
VFSDNLLLKRVNNLKMRIEIFWELREIIEKTDLLNNEFSLNTLLL
KYWYAIAVRYNLKEAIQYFYQKYTHLNTWRLTCALCFNNVFDLHE
AYEKDKICMDLEEMMRIACIKDHNLSTIYYCYMLGANINQAMLTS
IQYYNIENIFFCMDLGADAFEEGMALVGQEGYEPIRNILSLKIYS
PATTPLPKSTDPEIIDHALKNYFSKNMMVFLTYDLR*
MGF 360 13L protein sequences
Georgia 2007/1 MGF 360 13L protein
SEQ ID No. 170
MSLPLSLQTLVKKTTASQCLSTDEHCILKYCGLWWHDAPLKLCMD
RGRIQIKSGFLGEDIDLRVALIIAVKENNYSLIKLETEWGANINY
GLLSINTKHIRELCRQLGAKETLEDNDIFRIFTRIMHNKTSGSII
LCHEIFMNNPILENKFVIQLRGLIYKRLWGLIEIKETDELNGLLV
KYWYAKAVQYDCKDAICFLDEKYTDLNEWRLKCLLYYNKIYELHE
MYHKENIQIDVHDMICLASTKDNNPLTIYYCYALGGNINQAMLTS
VQYYNIGNIFFCIDLGGNAFEEGRMAEQKGYNFLSHSLALDIYSS
DASLPLNLKDPEEISSLLKDYKSKNLSIIWEYSHNIL*
China/2018/AnhuiXCGQ MGF 360 13L protein
SEQ ID No. 171
MSLPLSLQTLVKKTIASQCLSTDEHCILKYCGLWWHDAPLKLCMD
RGRIQIKSGFLGEDIDLRVALIIAVKENNYSLIKLFTEWGANINY
GLLSIMTKHIRELCRQLGAKETLEDNDIFRIFTRIMHMKTSGSIIL
CHEIFMNMPILENKFVIQLRGLIYKRLWGLIEIKETDELNGLLVK
YWYA
KAVQYDCKDAICFLDEKYTDLNEWRLKCLLYYNKIYELHEMYHKE
NIQIDVHDMICLASTKDNNPLTIYYCYALGGNINQAMLTSVQYYN
IGNIFFCIDLGGNAFEEGRAIAEQKGYNFLSHSLALDIYSSDASL
PLNLKDPEEISSLLKDYKSKNLSIIWEYSHNIL*
Tengani 62 MGF 360 13L protein
SEQ ID No. 172
MSLPLSLQTLVKKTVANQSLSTDEHCILKHCGLWWHDVPLKLCMD
RGQIQIKSGFLGEDIDLHIALIIAVKENNYSLIKLFTEWGANINY
SLLSINTKHIRELCRQLGAKETLEDDDIFRIFTKIMHNKTSGSII
LCHDIFMNNPNIEDKFTIQLRGLIYKRLWGLIEIKETDELNGLLV
KYWYAKAVQYECKDAICFLDEKYTDLNEWRLKCLLYYNKIYELHE
MYHKENIQIDVHDMICLASTKDNNPLTIYYCYALGGNINQAMLTS
VQYYNIGNIFFCIDLGGNAFEEGRAIAEQKGYNFLSHSLALDIYS
SDASLPLNLKDPEEISSLLKDYKSKNLSIIWEYSHNIL*
Warthog MGF 360 13L protein
SEQ ID No. 173
MSLPLSLQTLVKKTVASQCLSTDEHCILKHCGLWWHDVPLKLCMD
RGQIQIKSGFLGEDIDLHIALIIAVKENNYSLIKLFTEWGAHINY
SLLSINTEHIRELCRQLGAKETLEDNDIFRIFTKIMHNKTSGRII
LCHEIFMNNPNIENKFTIQLRGLICKRLWGLIEIKETDELNDLLV
KYWYAKAVQYECKDAICFLDEKYTDLNEWRLKCLLYYNKIYELHE
MYHKEKVQIDVHDMICLASTKDNNPLTIYYCYALGGNINQAMLTS
VQYYNIGNIFFCIDLGGNAFEEGRAIAEQKGYHFLSHSLTLDIYS
SDASLPLNLKDPEEISSLLKDYKSKNLSIIWEYSHNIL*
Warmbaths MGF 360 13L protein
SEQ ID No. 174
MSLPLSLQTLVKKTVASQCLSTDEHCILKHCGLWWHDVPLKLCMD
RGQIQIKSGFLGEDIDLHVALIIAVKENNYSLIKLFTEWGAHINY
SLLSINTEHIRELCRQLGAKKTLEDNDIFRIFTKIMHNKTSGRII
LCHEIFMNNPNIENKFTIQLRGLICKRLWRLIEIKETDELNDLLV
KYWYAKAVQYECKDAICFLDEKYTGLNEWRLKCLLYYNKIYELHE
MYHKDKVQIDVHDMICLASTKDNNPLTIYYCYALGGNINQAMLTS
VQYYNIGNIFFCIDLGGNAFEEGRAIAEQKGYHFLSHSLALDIYS
SDASLPLNLKDPEEISSLLKGYKSKNLSIIWEYSHNIL*
Pretorisuskop/96/4 MGF 360 13L protein
SEQ ID No. 175
MSLPLSLOTLVKKTVASQCLSIDEHCILKHCGLWWHDVPLKLCMD
RGQIQIKSGFLGEDIDLHIALIIAVKENNYSLIKLFTEWGAHTNY
SLLSINTEHTRELCRQLGAKETLEDDDVFRIFTKIMHNKTSGRII
LCHDIFMNNPNTENKFTTQLRGLICKRLWGLIEIKETDELNDLLV
KYWYAKAVQYECKDAICFLEEKYTDLNEWRLKCLLYFNKIYELHE
MYHKEKVQIDVHDMICLASTKDNNPLTTYYCYALGGNINQAMLTS
IQYYNTGNIFFCIDLGGNAFEEGRAIAEQKGYNFLSHSLALDIES
SDASLPLNLKDPEEISSFLKDYKSKNLSIIWEYSHNIL*
Mkuzi 1979 MGF 360 13L protein
SEQ ID No. 176
MSLPLSLQTLVKKTVASQCLSTDEHCILKYCGLWWHDAPLKLCMD
RGRIQIKSGFLGEDIDLRVALIIAVKENNYSLIKLETEWGANINY
SLLSINTKHIRELCRQLGAKETLEDNDIFRIFTRIMHNKTSGSII
LCHEIFMNNPILENKFVIQLRGLIYKRLWGLTEIKETDKLNDLLV
KYWYAKAVQYVCKNAICFLDEKYTDLNEWRLKCLLYYNKIYELHE
MYHKKKVOIDVHDMICLACAKDNNLLTIYYCYALGSNINQAMLTS
VQYYNTGNIFFCIDLGGNAFEEGRAIAEQKGYNFLSHSLTLDIYS
SDASLPLNLKDPEEISSLLKDYKSKNLSIIWEYSHNIL*
L60 MGF 360 13L protein
SEQ ID No. 177
MSLPLSLQTLVKKTVASQCLSTDEHCILKYCGLWWHDAPLKLCMD
RGRIQIKSGFLGEDIDLRVALIIAVKENNYSLIKLETEWGANINY
SLLSINTKHIRELCRQLGAKETLEDNDIFRIFTRIMHNKTSGSII
LCHEIFMNNPMLENKFVIQLRGLIYKRLWGLIEIKETDELNDLLV
KYWYAKAVQYVCKNAICFLDEKYTDLNEWRLKCLLYYNKIYELHE
MYHKKKVOIDVHDMICLACAKDNNLLTIYYCYALGGNINQAMLTS
VQYYNVGNIFFCIDLGGNAFEEGRAIAEQKGYNFLSHSLTLDIYS
SDASLPLNLKDPEKISSLLKDYKSKNLSIIWEYSHNIL*
Benin 97/1 MGF 360 13L protein
SEQ ID No. 7
MSLPLSLQTLVKKTVASQCLSTDEHCILKYCGLWWHDAPLKLCMD
RGRIQIKSGFLGEDIDLRVALIIAVKENNYSLIKLFTEWGANINY
SLLSINTKHIRELCRQLGAKETLEDNDIFRIFTRIMHNKTSGSII
LCHEIFMNNPMLENKFVIQLRGLIYKRLWGLIETKETDELNDLLV
RYWYAKAVQYVCKNAICFLDEKYTDLNEWRLKCLLYYNKIYELHE
MYHKKKVOIDVHDMICLACAKDNNLLTIYYCYALGGNINQAMLTS
VQYYNVGNIFFCIDLGGNAFEEGRAIAEQKGYNFLSHSLTLDIYS
SDASLPLNLKDPEKISSLLKDYKSKNLSIIWEYSHNIL*
Malawi Lil-20/1 (1983) MGF 360 13L protein
SEQ ID No. 178
MSAPLSLQTLVKKTVASTSCLSIDEHILKYCDLWWHDAPLKLYMD
RGRIQIKSGFLGEDIDLCVALIIAVKENNYNLIKLFTELGANINY
SLLSINTK
HVRDLCRQLGAKETLEDYDTFCIFNKIMHNKTSGSVILCHEIFIN
NPNLENKFAAQLRRLIYKRLCGLIETKETDELSELLVRYWYAKAV
QYDYKDAICFLDEKYTDLNEWRLKCYLYYNKIYELHDIYHKEKIQ
IDVNEMLSLACIRDNNPLTIYYCYALGGNINQAMLTSVQYYNIGN
IYFCIDLGGNAFEEGSAIARQKGYNFLSHSLVLNIYSSDASLPLN
LKDPEEISSLLKNYKSKNLSIILDYSHNIL*
Kenya 1950 MGF 360 13L protein
SEQ ID No. 179
MSSPLSLOTLVKKTVASTSCLSIDEHILKYCGLWWHDAPLKLYID
RGRIYIKSGELGEDIDLCVALIIAVKENNYSLIKLFTEWGAYINY
SLLSINTKHARDLCRQLGAKETLDDYDIFCIFNKIMHNKTSGSII
LCHEIFINNPKLENNFAAQLRRLIYKRLCGLIETKETDELSELLV
KYWYANAVQYDHKDAICFLDEKYTDLDEWRLKCYLCYNKIYELHD
IYHKKKIQIDVNEMLSLACIRDNNLLTIYYCYALGGNINQAMLTS
VQYYNIGNIYFCIDLGGNAFEEGSAIARQNGYNFLCHSLI
LNIYSSDASLPLNLWPEEISSLLKNYKSKNLSIILDYSHKIL*
Ken06.Bus MGF 360 13L protein
SEQ ID No. 180
MSSPLSLQTLVKKTVAGTSCLSIDEHILKYCGLWWHDAPLKLYID
RGRIYIKSGFLGEDIDLCVALIIAVKENNYSMIKLFTEWGAYINY
SLLSINTKHARDLCRQLGAKETLDDYDIFCIFNKIMHNKTSGSII
LCHEIFINNPNLENNFAAQLRRLIYKRLCGLIETKETDELSELLV
KYWYANAVQYDHKDAICFLDEKYTDLDEW*LKCYLCYNKIYELHD
IYHKEKIQIDVNEMLSLACIRDNNLLTIYY
CYALGGNINQAMLTSVQYYNIGNIYFCIDLGGNAFEEGSAIARQN
GYNFLSHSLVLNIYSSDASLPLNLKDPEEISSLLKNYKSKNLSII
LDYSHKIL*
MGF 360 14L protein sequences
Georgia 2007/1 MGF 360 14L protein
SEQ ID No. 181
MLSLQTLAKKVVACNYLSSDYDYTLQRFGLWWDLGPIHLCNNCKQ
VFSYKHLQCFSEDDLCLEAALVKAVKSDNLELIRLFVDWGANPEY
GLIRVPAVYLKRLCAELGGLTPVSEPRLLEILKEVARLKSCAGVL
LGYDMFCHNPLLETVTRTTLDTVTYTCSNIPLTGDTAHHLLTKFW
FALALRHNFTKAIHYFYKRHKNHLYWRVACSLYFNNIFDIHELCR
EKEICISPNLMMKFACLREKNYAAIYYCHRLGASLDYGMNLSIYN
NNTLNMFFCIDLGAADFDRAQLIAHKAYMYNLSNIFLVKQLFSRD
VTLVLDVTEPQEIYDMLKTYTSKNLKRAEEYLTAHPEIIVID*
China/2018/AnhuiXCGQ MGF 360 14L protein
SEQ ID No. 182
MLSLQTLAKKVVACNYLSSDYDYTLQRFGLWWDLGPIHLCNNCKQ
VFSYKHLQCFSEDDLCLEAALVKAVKSDNLELIRLFVDWGANPEY
GLIRVPAVYLKRLCAELGGL
TPVSEPRLLEILKEVARLKSCAGVLLGYDMFCHNPLLETVTRTTL
DTVTYTCSNIPLTGDTAHHLLTKFWFALALRHNFTKAIHYFYKRH
KNHLYWRVACSLYFNNIFDIHELCREKEICISPNLMMKFACLREK
NYAAIYYCHRLGASLDYGMNLSIYNNNTLNMFFCIDLGAADFDRA
QLIAHKAYMYNLSNIFLVKQLFSRDVTLVLDVTEPQEIYDMLKTY
TSKNLKRAEEYLTAHPEIIVID*
Tengani 62 MGF 360 14L protein
SEQ ID No. 183
MLSLQTLAKKVVACNYLSSDYDYTLQRFGLWWDLGPIHLCNNCKQ
IFSYKHVQCFSEDDLCLEAALVKAVKSDNLELIRLFVDWGANPEY
GLIRVPAVHLKRLCTELGGLTPVSESRLLEILKEVARLKSCAGVL
LGYDMFCHNPLLETVTRTTLDTVTYTCSNIPLTGDTAHHLLTKFW
FALALRHNFTKAIHYFYKRHKNHLYWRVACSLYFNNIFDIHELCR
EKEICISPNLMMKFACLQKKNYAAIYYCYRLGASLDYGMNLSIYN
NNTLNMFFCIDLGATDFDRAQRIAHKAYMYNLSNIFLVKQLFSRD
VTLALDVTEPQEIYDMLKSYTSKNLKRAEEYLTAHPEIIVID*
Warthog MGF 360 14L protein
SEQ ID No. 184
MLSLQTLAKKVVACNYLSSDYDYMLQRFGLWWDLGPIHLCNNCKQ
IFSYKHLQCFSEDDLCLEAALVKAVKSDNLELIRLFVDWGANPEY
GLIRVPAVHLKRLCTELGGLTPVSEPRLLEILKEVAKLKSCAGVL
LGYDMFCHNPLLETVTRTTLDTVTYTCSNIPLTGDTAHHLLTKFW
FALALRHNFTKAIHYFYKRHKNHLYWRVACSLYFNNIFDIHELCR
EKEICISPNLMMKFACLRKKNYAAIYYCYRLGASLDYGMNLSIYN
NNTLNMFFCIDLGATDFDRAQRIAHKTYMYNLSNIFLVKQLFSRD
VTLALDVTEPQEIYDMLKSYTSKNLKRAEEYLTAHPEIIVID*
Warmbaths MGF 360 14L protein
SEQ ID No. 185
MLSLQTLAKKVVACNYLSSDYDYMLQRFGLWWDLGPIHLCNNCKQ
IFSYKHLQCFSEDDLCLEAALVKAVKRDNLELIRLFVDWGANPEY
GLICVPTVHLKRLCTELGGLTPVSEPRLLEILKEVANLKSCAGVL
LGYDMFCYNPLLETITRTTLDTVMYSCSKIPLMGDTAHHLLTKFW
FALALRHNFTKAIHYFYKRHKNHLYWRVACSLYFNNIFDIHELCR
EKEICISPNLMMKFACLREKNYAAIYYCYRLGASLDYGMNLSIYN
NNSLNLFFCIDLGATDFDRAQRIAHKAYMYNLSNILLVKQLFSRD
VTLALDVTEPQEIYDRLKAYTSKNMKRAEEYLTAHPEIIVID*
Pretorisuskop/96/4 MGF 360 14L protein
SEQ ID No. 186
MLSLQTLAKKVVACNYLSSDYDYTLQRFGLWWDLGPIHLCNNCKQ
IFSYKHLQCFSEDDLCLEAALVKAVKSDNLELIRLFVDWGANPEY
GLIRVPAVHLKRLCTELGGLTPVTEPRLLEILKEVAKLKSCAGVL
LGYDMFCHNPLLETVTRTTLDTVTYTCSNIPLTGDTAHLLLTKFW
FALALRHNFTKAIHYFYKRHKNHLYWRVACSLYFNNIFDIHELCR
EKEICISPNLMMKFACLQKKNYAAIYYCYRLGASLDYGMNLSIYN
NNTLNMFFCIDLGATDFDRAQHIAHKAYMYNLSNIFLVKQLFSRD
VTLALDVTEPQEIYDRLKSYTSKNLKRAEEYLTAHPEIIVID*
Mkuzi 1979 MGF 360 14L protein
SEQ ID No. 187
MLSLQTLAKKVVACNYLSSDYDYTLQRFGLWWDLGPIHLCNNCKQ
VFSYKHLQCFSEDDLCLEAALVKAVKSDNLELIRLFVDWGANPEY
GLIRVPAVYLKRLCAELGGLTPVSEPRLLEILKEVANLKSCAGVL
LGYDMFCHNPLLETVTRTTLDTVTYTCSNIPLTGDTAHLLLTKFW
FALALRHNFTKAIHYFYKRHKNQLYWRVACSLYFNNIFDIHELCR
EKEICISPNLMMKFACLREKNYAAIYYCHRLGASLDYGMNLSIYN
NNTLNMFFCIDLGAADFDRAQLIAHKAYMYNLSNIFLVKQLFSRD
VTLVLDVTEPQEIYDMLKTYTSKNMKRAEEYLTAHPEIIVID*
L60 MGF 360 14L protein
SEQ ID No. 188
MLSLQTLAKKVVACNYLSSDYDYTLQRFGLWWDLGPIHLCNNCKQ
VFSYKHLQCFSEDDLCLEAALVKAVKSDNLELIRLFVDWGANPEY
GLIRVPAVYLKRLCAELGGLTPVSEPRLLEILKEVANLKSCAGVL
LGYDMFCHNPLLETVTRTTLDTVTYTCSNIPLTGDTAHLLLTKFW
FALALRHNFTKAIHYFYKRHKNQLYWRVACSLYFNNIFDIHELCR
EKEICISPNLMMKFACLREKNYAAIYYCHRLGASLDYGMNLSIYN
NNTLNMFFCIDLGAADFDRAQLIAHKAYMYNLSNIFLVKQLFSRD
VTLVLDVTEPQEIYDMLKTYTSKNMKRAEEYLTAHPEIIVID*
Benin 97/1 MGF 360 14L protein
SEQ ID No. 8
MLSLQTLAKKVVACNYLSSDYDYTLQRFGLWWDLGPIHLCNNCKQ
VFSYKHLQCFSEDDLCLEAALVKAVKSDNLELIRLFVDWGANPEY
GLIRVPAVYLKRLCAELGGLTPVSEPRLLEILKEVANLKSCAGVL
LGYDMFCHNPLLETVTRTTLDTVTYTCSNIPLTGDTAHLLLTKFW
FALALRHNFTKAIHYFYKRHKNQLYWRVACSLYFNNIFDIHELCR
EKEICISPNLMMKFACLREKNYAAIYYCHRLGASLDYGMNLSIYN
NNTLNMFFCIDLGAADFDRAQLIAHKAYMYNLSNIFLVKQLFSRD
VTLVLDVTEPQEIYDMLKTYTSKNMKRAEEYLTAHPEIIVID*
Malawi Lil-20/1 MGF 360 14L protein
SEQ ID No. 189
MLSLQTLAKKVVACNYLSSDYDYTLQRFGLWWDLGPIHLCNTCKQ
IFSYKHLQCFSEDDLCLEAALVKAVKSDNLELIRLFVDWGANPEY
GLIRVPAVHLKRLCMELGGLTPVSESRLLEILKEVADLKSCAGVL
LGYDMFCHNPLLETVTRTTLDTVMYTRSKIPLTGDTAHLLLSKFW
FALALRHNFTKAIHYFYEKHKNQLYWRLTCSLYFNNIFDLHELCC
KKEICISPNLMMKFACLREENYAAIYYCHMLGASLDYGMNLSIYN
NNTLNLFFCIDLGATNFDRARLIARRVYMYNLSNLFLVKQLFSRD
VSLILDLTEPQAIYDMLNTYTSKNLKQAEEYFTAHPEIVVID*
Kenya 1950 MGF 360 14L protein
SEQ ID No. 190
MLSLQTLAKKVVACNYLSSDYDYTLQRFGLWWDLGPIHLCNTCKQ
IFSYQHLQCFSEDDLCLDAALVKAVKSDNLELIRLFVDWGANPEY
GLIRVPAVDLKRLCTELGGLTPVSESRLLEILKEVADLKSCAGVL
LGYDMFCHNPLLETVTRTTLNTVMYTRSKIPLTGDTAHLLLTKFW
FALALRHNFTKAIHYFYEKHKNQLYWRLACSLYFNNIFDLHELCS
KKEICISPNLMMKFACLREENYAAIYYCHMLGASPDYGMNLSIYN
NNTLNLFFCIDLGATNFDRARLIAHRVYMYNLSNIFLVKQLFSRD
ASLVLNITEPQAIYDMLKTYTSKNLKRAEEYFTAHPEIVVID*
Ken06.Bus MGF 360 14L protein
SEQ ID No. 191
MLSLQTLAKKVVACNYLSSDYDYTLQRFGLWWDLGPIHLCNTCKQ
IFSYQHLQCFSVDDLCLDAALVKAVKSDNLELIRLFVDWGANPEY
GLIRVPAVHLKRLCTELGGLTPVSESRLLEILKEVADLKSCAGVL
LGYDMFCHNPLMETVTRTTLNTVMYTRSKIPLTGDTAHLLLTKFW
FALALRHNFTKAIHYFYEKHKNQLYWRLTCSLYFNNIFDLHELCS
KKEICISPNLMMKFACLREENYAAIYYCHMLGASPDYGMNLSIYN
NNTLNLFFCIDLGATNFDRARLIAHRVYMYNLSNIFLVKQLFSRD
ASLVLNITEPQAIYDMLKTYTSKNLKRAEEYFTAHPEIVVID*
MGF 505 1R protein sequences
Georgia 2007/1 MGF 505 1R protein
SEQ ID No. 192
MFSLQNLCRKTLPNRKLPEFFDEYILQLLGLYWENHGTIQRAGNN
CVLIQQHTLIPVNEALRTAASEENYEIVSLLLAWEGNLYYAIIGA
LEGNRHDLIRKYDDQIKDHHEILPFIDDPVIFHKCHIMRQCFFDC
ILYQAVKYSKFRVLLYFKHRLEDDLPFTHLLIEKACKDHNYEVIK
WIYENLHIYNMIDTFECAIAHKDLHLYCLGYRFIYNRIVPDKYHH
LDIRMLSSLQLLHKVAAKGYLDFILETLKYDHNKDNINIILTQAA
TYNHRKILIYFIPQSTHAQIEQCLLVAIKAKSSRKTLNLLLSHLN
LSINLIKKISHYVATYNSTNIIGILSMRRKKKIYLDIILTKFVKK
AIFNKFVVRCMDTFSINPERILKIAARINRMMLVKKISEHVWKNH
AVRLKYLKHAVHTMKHKDGKNRLMNFIYDRCYYHMQGEEIFSLAR
FYAIHHAPKLFDVFYDCCILDTIRFKSLLLDCSHIIGKNAHDATN
INIVNKYIGNLFVMGVLSKKEILQDYPSIYSKQYMP*
China/2018/AnhuiXCGQ MGF 505 1R protein
SEQ ID No. 193
MFSLQNLCRKTLPNRKLPEFFDEYILQLLGLYWENHGTIQRAGNN
CVLIQQHTLIPVNEALRTAASEENYEIVSLLLAWEGNLYYAIIGA
LEGNRHDLIRKYDDQIKDHHEILPFIDDPVIFHKCHIMRQCFFDC
ILYQAVKYSKFRVLLYFKHRLEDDLPFTHLLIEKACKDHNYEVIK
WIYENLHIYNMIDTFECAIAHKDLHLYCLGYRFIYNRIVPDKYHH
LDIRMLSSLQLLHKVAAKGYLDFILETLKYDHNKDNINIILTQAA
TYNHRKILIYFIPQSTHAQIEQCLLVAIKAKSSRKTLNLLLSHLN
LSINLIKKISHYVATYNSTNIIGILSMRRKKKIYLDIILTKFVKK
AIFNKFVVRCMDTFSINPERILKIAARINRMMLVKKISEHVWKNH
AVRLKYLKHAVHTMKHKDGKNRLMNFIYDRCYYHMQGEEIFSLAR
FYAIHHAPKLFDVFYDCCILDTIRFKSLLLDCSHIIGKNAHDATN
INIVNKYIGNLFVMGVLSKKEILQDYPSIYSKQYMP*
Tengani 62 MGF 505 1R protein
SEQ ID No. 194
MFSLQNLCRKTLPDCKLPEFFDEYILQLLGLYWENHGTIQRAGNN
CVLIQQHNLIPVNEALRIAASEENYEIVSLLLAWEGNLYYAIIGA
LEGNRHDLIRKYDDQIKDHHEILPFIDDPVIFHKCHIMRQCFFDC
ILYQAVKYSKFRVLLSFKHRLRDDLPFTHLLIEKACKDHNYEVIK
WIYENLHIYNMIDTFECAIAHKDLRLYCLGYRFIYNRIVPDKYHH
LDIRMLSSLQLLHKVAAKGYLDFILETLKYDHNKDNINIILTQAA
TYNHRKILIYFIPQSTHAQIEQCLLVAIKTKSSKKTLNLLLSHLN
LSINLIKKISHYVATYNSTNIIGILSMRRKKKIYLDIILTKFVKK
AIFNKFVVRCMDTFSINPERILKIAARINRTMLVKKISEHVWKNH
AVRLKYLKHAVHTMKHKDGKNRLMNFIYDRCYYHMQGEEIFSLAR
FYAIHHAPKLFDVFYDCCILDTIRFKSLLLDCSHIIGKNAHDATN
INIVNKYIGNLFVMGVLSKKEILQDYPSIYSKHYMP*
Warmbaths MGF 505 1R protein
SEQ ID No. 195
MFSLQNLCRKTLPDCKLPEFFDEYILQLLGLYWENHGTIQRAGNN
CVLIQQRTLIPVNEALRIAASEENYEIVGLLLAWEGNLYYAIIGA
LEGNRHDLIRKYDDQIKDHHEILPFIDDPVIFHKCHIIRRCFFNC
ILYQAVKYSKFRVLLYFKHRLEDDLPLTHLLIEKACED
HNYEVIKWIYENLHTYDIMDTFECAIAHKDLRLYCLGYTFIYNRI
VPYEYHHLDILILSSLQLLHKVAAKGYLDFILETLKYDHNNDNLD
IILTQAATYNHRKILTYFIPQLTYAQIEQCLFMAIKRKSSKKTLN
LLLSHLTLSIELIKKISQYVVTYNSTNIIGILSMKRKKKIYLDIM
LTKYVKYAIFNKYVVRCMDRFSINPERIIKMAARINRMMLVKKIS
EHVWKNHAARLKHLKHAVHTMKHKDGKNRLMNFIYDRCYYHMQGE
EIFSLARFYAIHHAPKLFDVFYDCCILDTIQFKSLLLDCSHIIGK
NAHDATNINIVNKYIGDLFAMGVLSKKEILQDYPSIYSKHYML*
Mkuzi1979 MGF 505 1R protein
SEQ ID No. 196
MFSLQNLCRKTLPDCKLPEFFDDYILQLLGLYWENHGTIQRAGNN
CVLIQQHTLIPINEALRTAASEENYEIVSLLLAWEGNLYYAIIGA
LEGNRHDLIRKYDDQIKDHHEILPFIDDPVIFHKCHIMRRCFFDC
ILYQAVKYSKFRVLLYFKYRLEDDLPLTHLLIEKACENHNYEVIR
WIYENLHIYNMIDTFECAIAHKDLRLYCLGYTFIYNRIVPNKYHH
IDILILSSLQLLHKVAAKGYLDFILETLKYDHNNDNLDIILTQAA
TYNHRKILTYFIPQSTYAQIEQCLMVAIKTKSSKKTLNLLLSHLN
LSIKLIKKISQYVVTYNSTNIIGILSMKRKKKIYLDILLTKFVKN
AIFNKFVVRYMDTFSINPEKIVKMAARINKMMLVKKISEHIWKNH
AARLEHLKHAVHTMKHKDGKNRLMNFIYEYCYYHMQGEEIFSLAR
FYAIHHAPKLFDVFYNCCILDTIRFKSLLLDCSHIIGKNAHDATN
INIVNKYIDNLFAMGVLSKKEILQDYPSIYSKHYMP*
Warthog MGF 505 1R protein
SEQ ID No. 197
MFSLQNLCRKTLPDCKLPEFFDEYILQLLGLYWENHGTIQRAGNN
CVLIQQHTLIPVNEALRIAASEENYEIVSLLLAWEGNLYYAIIGA
LEGNRHDLIRKYDDQIKDHHEILPFIDDPVIFHKCHIMRRCFF
DCILYQAVKYSKFRVLLYFKYRLENDLPLAHLLIKKACEDHNYEV
IKWIYENLHIYNIMDTFGCAIAHKDLRLYRLGYTFIYNRIVPYKY
HYLDVLILSGLHLLYKVAAKGYLDFILETLKYDHNNDNLDIILTQ
AATYNHRKILTYYIPQLTYAQIEQCLFMAIKKKSSKKTLNLLLSH
LKLSIKLIKKISQYVATYNSTNIIGILNMRRKKKIYLDIILTKFV
KKAIFNKFVVRCMDTFSINPERIIKMAARINKMLLVKKISEHAWK
NHAARLKHLKHAVYTMKHKDGKNRLMNLIYDHYYYHMQGEEIFSL
ARFYAIHHAPKLFDVFYDCCLLDTIRFKNLLLDCSHIIGKNAHDA
TNITIVNKYIGNLFAMGVLSKKEILQDYPSIYSKHYMP*
L60 MGF 505 1R protein
SEQ ID No. 198
MFSLQNLCRKTLPDCKLPEFFDDYILQLLGLYWENHGTIQRAGNN
CVLIQQHTLIPVNEALRIAASEENYEIVGLLLAWEGNLYYAIIGA
LEGNRYNLIRKYDDQIKDHHDILPFIDDPIIFHKCHIMRRCFFDC
ILYQAVKYSKFRVLLYFKYTLEDDLPLVHLLIEKACEDHNYEVIK
WIYENLHVCHIIDTFDCAIAHKDLRLYCLGYTFIYNRIVPYKYHH
LDILILSSLQLLHKVAAKGYLDFILETLKYDHNIDNLDVILTQAA
TYNHRKILTYFIPQSTYAQIEQCLFVAIKTKSSKKTLNLLLSHLN
LSIKLIQKISQYVATFNSTNIIGILSMKRKKKIYLDIILTKFVKN
AIFNKFVVRCMERFSINPERIVKMAARINKMMLVKKISEHVWKNH
AARLKHLKHAVHTMKHKDGKNRLMNFIYEHCYYHMQGEEIFSLAR
FYAIHHAPKLFDVFYNCCILDTIRFKSLLLDCSHIIGKNAHDATN
INIVNKYIGNLFAMGVLSKKEILQDYPSIYSKHYMP*
Benin 97/1 MGF 505 1R protein
SEQ ID No. 9
MFSLQNLCRKTLPDCKLPEFFDDYILQLLGLYWENHGTIQRAGNN
CVLIQQHTLIPVNEALRIAASEENYEIVGLLLAWEGNLYYAIIGA
LEGNRYNLIRKYDDQIKDHHDILPFIDDPIIFHKCHIMRRCFFDC
ILYQAVKYSKFRVLLYFKYTLEDDLPLVHLLIEKACEDHNYEVIK
WIYENLHVCHIIDTFDCAIAHKDLRLYCLGYTFIYNRIVPYKYHH
LDILILSSLQLLHKVAAKGYLDFILETLKYDHNIDNLDVILTQAA
TYNHRKILTYFIPQSTYAQIEQCLFVAIKTKSSKKTLNLLLSHLN
LSIKLIQKISQYVATFNSTNIIGILSMKRKKKIYLDIILTKFVKN
AIFNKFVVRCMERFSINPERIVKMAARINKMMLVKKISEHVWKNH
AARLKHLKHAVHTMKHKDGKNRLMNFIYEHCYYHMQGEEIFSLAR
FYAIHHAPKLFDVFYNCCILDTIRFKSLLLDCSHIIGKNAHDATN
INIVNKYIGNLFAMGVLSKKEILQDYPSIYSKHYMP*
Pretorisuskop/96/4 MGF 505 1R protein
SEQ ID No. 199
MFSLQNLCRKTLPDCKLPEFFDEYILQLLGLYWENHGTIQRAGNN
CVLVQQHTLIPVNEALRIAASEENYEIVSLLLAWEGNLYYAIIGA
LEGNRHDLIRKYDDQIKDHHEILPFIDNPVIFHKCHIMRRCFFDC
ILYQAVKYSKFRVLLYFKYRLENDLPLAHLLVEKACEDHNYEVIK
WLYENLHIYNIMETFECAIAHKDLRLYRLGYTFIYNRIVPYKYHY
LDVLILSGLHLLYKVAAKGYLDFILETLKYDHNNDNLDIILTQAV
TYNHRKILTYYIPQLTYAQIEQCLFMAIKKKSSKKTLNLLLSHLK
LSIKLIKKISQYVATYNSTNIIGILNMKRKKKIYLDIILTKFVKY
AIFNKYVVRCMDTFSINPERIIKMAARINKMLLVKKISQHAWKNH
AARLKHLKHAVYTMKHKDGKNRLMNLIYDHYYYHMQGEEIFSLAR
FYAIHHAPKLFDVFYDCCLLDTIRFKSLLLDCSHIIGKNAHDATN
ITIVNKYIGNLFAMGVLSKKEILQDYPSIYSKHYMP*
Kenya 1950 MGF 505 1R protein
SEQ ID No. 200
MFSLQNLCRKTLPDRKLPEFFDDYVLQLLGLYWENHGTIQRAGNN
CVLIQQHNLIPVNEALRIAASEENYEIVSLLLAWEGNLYYAIIGA
LEGNRHNLIRKYDDQIKDHHEILPFIDDPVIFHKCHMMRRCFF
NCILYQAVKYSKFSVLLYFKYILKENLPLVHSLIEKACEDHNYEV
IKWIYENLHIYEIMDTFKCAIAHKDLHLYSLGYTFIYNRIVPYKY
HHLDIRILSRLQLLHKVTAKGYLDFILETLKYDHNKDNINIILTQ
AATYNHRNILTYFIPQSTYAQIEQCLFVAIKTNASKKTLNLLLSH
LNLSIKLVKKLSQYVVAYKSTNIISILSMQQKKKIYLDIILTKVV
KNAIFIKFVIGCMVTFSINPERIVKMAARIKKMKLVKNISEHVWK
NHAAKLKHLKHAVHTMKHQEGKNRLMNFIYDHCYYHMQGEEIFSL
ARFYAIHHAPKLFDVFYDCCILDTIRFKSLLLDCSYIIAKNAHDA
SINIVNKYIGNLFAMGVLSKKEILQDYPSIYSKDYML*
Ken06.Bus MGF 505 1R protein
SEQ ID No. 201
MFSLQNLCRKTLPDCKLPEFFDDYVLQLLGLYWENHGTIQRAGNN
CVLIQQHNLIPVNEALRIAASEENYEIVSLLLAWEGNLYYAIIGA
LEGNHHNLIRKYDDQIKDHHEILPFIDDPVIFHKCHTMRRCFFNC
ILYQAVKYSKFSVLLYFKYILKENLPLVHSLIEKAYKYHNYEVIK
WIYENLHIYDIINTFKCAIAHKDLRLYCLGYTFVYNRIVPYKYYH
LDIRILLRLQLLHKVTAKGYLDFILETLKYDHNTNNIDIILTQAA
TYNHRNILTYFIPQSTYAQIEQCLFVAIKTNASKKTLNLLLSHLN
LSIKLVKKLSQYVVAYKSTNIISILSRQQKKKIYLDIILTKVVKN
AVFNKFVIGCMVTFSINPERIVKMAARIKKMKLVKNISEHVWKNH
AVKLKYLKHAVHTMKHQEGKNRLMNFIYDHCYYHMQEEEIFSLAR
FYAIHHAPKLFDVFYDCCILDTIRFKSLLLDCSHIIVKNAHDASI
NIVNKYIGNLFAMGVLSKKEILQDYPSIYSKDYML*
Ken05/Tk1 MGF 505 1R protein
SEQ ID No. 202
MFSLQNLCRKTLPDCKLPEFFDDYVLQLLGLYWENHGTIQRAGNN
CVLIQQHNLIPVNEALRIAASEENYEIVSLLLAWEGNLYYAIIGA
LEGNRHNLIRKYDDQIKDHHEILPFIDDPVIFHKCHIMRRCFFNC
MLYQAVKYSKFSVLLYFKYILKENLPLVHSLIEKAYKYHNYEVIK
WIYENLHIYDIINTFKYAIAHKDLRLYCLGYTFVYNRIVPYKYYH
LDIRILLRLQLLHKVTAKGYLDFILETLKYDHNTNNIDIILTQAA
TYNHRNILTYFIPQSTYAQIEQCLFVAIKTNASKKTLNLLLSHLN
LSIKLVKKLSQYVVAYKSTNIISILSMQQKKKIYLDIILTKVVKN
AVFIKFVIGCMVTFSINPERIVKMAARIKKMKLVKNISEHVWKNH
AAKLKHLKHAVHTMKHQEGKNRLMNFIYDHCYYHMQGEEIFSLAR
FYAIHHAPKLFDVFYDCCILDTIRFKSLLLDCSHIIAKNAHDASI
NIVNKYIGNLFAMGVLSKKEILQDYPSIYSKYDIL*
Malawi Lil-20/1 (1983) MGF 505 1R protein
SEQ ID No. 203
MFSLQNLCRKTLPDCKLPEFFDEYILQLLGLYWENHGTIQRAGNN
CVLIQQHNLIPVNEALRIAASEENYEIVSLLLAWEGNLYYAIIGA
LEGNRPDLIRKYDDQIKDHHEILPFIDDPIIFHKCHIMRRCFFNC
ILYQAVKYSKFRVLLYFKHRLGDDLPLTHLLIEKACEDHNYEVIK
WIYENLHSYNIMDTFECAIAHKDLRLYCLGYTFIYNRIVPYKYHH
LDICILSSLQLLHKVAAKGYLDFILETLKYDHNINNIDIILTQAA
TYNHRKILTYFIPQLTYAQIEQCLLVAIKTKASKKTLNLLLSHLN
LSIKLIKKISQYVVTYNSTNIISILSMRRKKKIYLDIILTEFVKN
AIFNKFVVRCMDTFSINPERIVKMAARINRMMLVKNISERVWKNH
AVKLKHLKHAVHTMKHQEGKNRLMNFIYDHCYYHMQGEEIFGLAR
FYAIHHAPKLFDVFYDCCMLDATRFKSLLLDCPHIIGKNAYDAGI
NLVNKYIGNLFAMGVLSKKEILQDYPSIYSKHDMF*

### DP148R

The DP148R gene is located close to the right end of the ASFV genome, at position 177915 to 178679 on the Benin 97/1 genome. The DP148R gene may also be referred to as MGF 360 18R. DP148R is expressed at early times post-infection. The amino acid sequence of the DP148R protein has no significant similarity to other proteins; the secondary structure is predicted to be predominantly helical, but no signal peptide or transmembrane domains are evident.

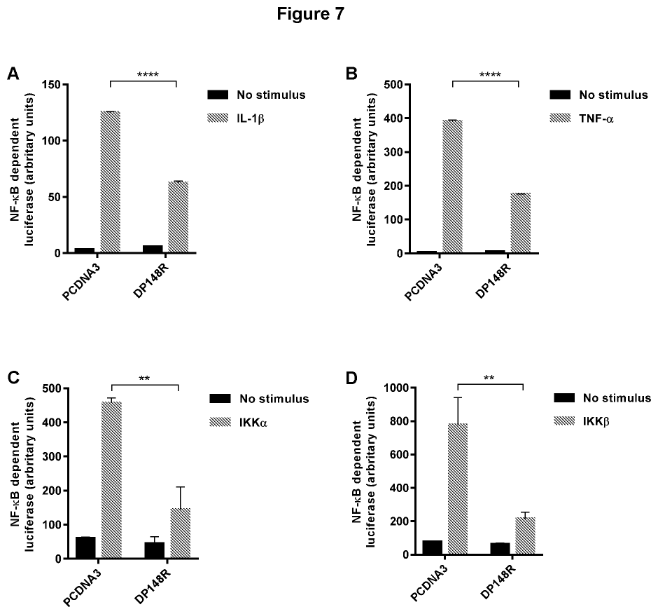

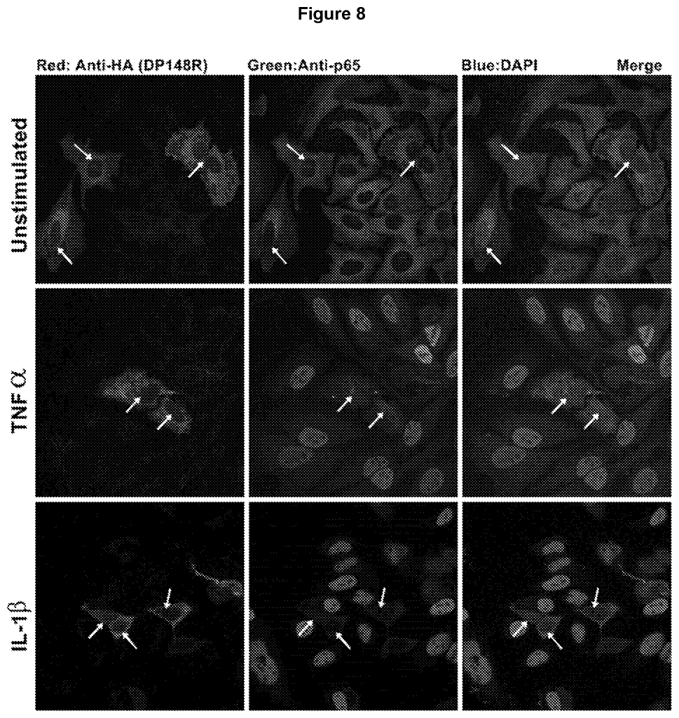

DP148R inhibits type I interferon. DP148R also inhibits activation of the NF-κB transcription factor (see FIG. 7). DP148R inhibits nuclear translocation of the p65 subunit of NF-κB (see FIG. 8). NF-κB controls expression of interferon and pro-inflammatory cytokines as part of the host's innate immune system response to viral infection. The inhibition of NF-κB by DP148R would results in decreased amounts of type I interferon (IFN) and pro-inflammatory cytokines and chemokines produced by cells infected with ASFV and allow ASFV to circumvent the host innate immune response, favouring virus replication and disrupting the development of adaptive responses.

The gene (i.e. nucleotide) sequences and positions in the genome of DP148R genes from different ASFV strains are presented below.

Georgia 2008/1 DP148R
(MH910495.1:183350-184063)
SEQ ID No. 287
ATGTTAGAAATAGTATTGGCAACGCTGCTAGGCGA
CCTGCAGCGGCTCCGGGTTCTTACCCCTCAGCAGC
GGGCAGTTGCCTTCTTTCGAGCCAATACTAAGGAG
CTAGAGGACTTCTTATGCTCAGATGGGCAGTCTGA
GGAGGTACTGTCTGGCCCCCTTCTTAACCGTCTAC
TAGAACCCTCAGGCCCTCTTGATATTTTAACCGGA
TATCACCTATTTCGTCAGAATCCCAAGGCAGGTCA
GTTGCGCGGCCTTGAGGTCAAGATGCTTGAACGGT
TATACGATGCTAATATTTACAATATACTGTCTCGG
CTGCGGCCTGAAAAAGTTCGCAACAAGGCTATTGA
GCTATACTGGGTTTTCCGAGCTATCCATATTTGTC
ATGCTCCTTTAGTTTTAGATATTGTACGATATGAG
GAACCGGACTTTGCTGAACTGGCCTTTATTTGTGC
TGCTTACTTTGGTGAACCTCAGGTAATGTATTTGC
TCTACAAATATATGCCTCTGACCCGCGCAGTTCTT
ACGGATGCCATCCGGATAAGTCTTGAGAGCAACAA
CCAGGTAGGGATTTGCTATGCTTACTTGATGGGAG
GCAGCCTCAAGGGACTAGTCTCCGCCCCACTGCGT
AAACGTCTGCGCGCCAAACTACGCTCGCAGCGCAA
AAAGAAGGACGTTCTTTCACCCCACGACTTCTTAC
TGCTGCTCCAGTAG
China/2018/AnhuiXCGQ DP148R
(MK128995.1:183372-184085)
ATGTTAGAAATAGTATTGGCAACGCTGCTAGGCGA
SEQ ID No. 288
CCTGCAGCGGCTCCGGGTTCTTACCCCTCAGCAGC
GGGCAGTTGCCTTCTTTCGAGCCAATACTAAGGAG
CTAGAGGACTTCTTATGCTCAGATGGGCAGTCTGA
GGAGGTACTGTCTGGCCCCCTTCTTAACCGTCTAC
TAGAACCCTCAGGCCCTCTTGATATTTTAACCGGA
TATCACCTATTTCGTCAGAATCCCAAGGCAGGTCA
GTTGCGCGGCCTTGAGGTCAAGATGCTTGAACGGT
TATACGATGCTAATATTTACAATATACTGTCTCGG
CTGCGGCCTGAAAAAGTTCGCAACAAGGCTATTGA
GCTATACTGGGTTTTCCGAGCTATCCATATTTGTC
ATGCTCCTTTAGTTTTAGATATTGTACGATATGAG
GAACCGGACTTTGCTGAACTGGCCTTTATTTGTGC
TGCTTACTTTGGTGAACCTCAGGTAATGTATTTGC
TCTACAAATATATGCCTCTGACCCGCGCAGTTCTT
ACGGATGCCATCCGGATAAGTCTTGAGAGCAACAA
CCAGGTAGGGATTTGCTATGCTTACTTGATGGGAG
GCAGCCTCAAGGGACTAGTCTCCGCCCCACTGCGT
AAACGTCTGCGCGCCAAACTACGCTCGCAGCGCAA
AAAGAAGGACGTTCTTTCACCCCACGACTTCTTAC
TGCTGCTCCAGTAG
OURT 88/3 DP148R
(NC_044957.1:168827-169045)
SEQ ID No. 289
ATGCAAAATAAAATCCCAAATTTTAACCTTTTCTT
TTTTTTTCTATACAGGATGTTAGAAATAGTATTGG
CAACGCTGCTAGGCGACCTGCAGCGGCTCCGGGTT
CTTACCCCTCAGCAGCGGGCGGTTGCCTTCTTTCG
AGCCAATACTAAGGAGCTAGAGGACTTCTTACGCT
CAGATGGGCAATCTGAGGAGATACTGTCTGGCCCC
CCTCCTTAA
L60 DP148R
(NC_044941.1:177973-178737)
SEQ ID No. 290
ATGCAAAATAAAATCCCAAATTTTAACCTTTTCTT
TTTTTTTCTATACAGGATGTTAGAAATAGTATTGG
CAACGCTGCTAGGCGACCTGCAGCGGCTCCGGGTT
CTTACCCCTCAGCAGCGGGCGGTTGCCTTCTTTCG
AGCCAATACTAAGGAGCTAGAGGACTTCTTACGCT
CAGATGGGCAATCTGAGGAGATACTGTCTGGCCCC
CTCCTTAACCGTCTACTAGAACCCTCATGCCCTCT
TGATATTTTAACCGGATATCACCTATTTCGTCAGA
ATCCCAAGGCAGGTCAGTTGCGCGGCCTTGAGGTC
AAGATGCTTGAACGGTTATACGATGCTAATATTTA
CAATATATTGTCTCGGCTGCGACCTGAAAAAGTCC
GCAACAAGGCTATTGAGCTATACTGGGTTTTCCGA
GCTATCCATATTTGTCATGCTCCTTTAGTTTTAGA
TATTGTACGATATGAGGAACCGGACTTTGCTGAAC
TGGCCTTTATTTGTGCTGCTTACTTTGGTGAACCT
CAGGTAATGTATTTGCTCTACAAATATATGCCTCT
GACCCGCGCAGTTCTTACGGATGCCATCCAGATAA
GTCTTGAGAGCAACAACCAGGTAGGGATTTGCTAT
GCTTACTTGATGGGAGGCAGCCTCAAGGGACTAGT
CTCCGCCCCACTGCGTAAACGTCTGCGCGCCAAAC
TACGCTCGCAGCGCAAAAAGAAGGACGTTCTTTCA
CCCCACGACTTCTTACTGCTGCTCCAGTAG
Benin 97/1
DP148R (NC_044956.1:177915-178679)
SEQ ID No. 291
ATGCAAAATAAAATCCCAAATTTTAACCTTTTCTT
TTTTTTTCTATACAGGATGTTAGAAATAGTATTGG
CAACGCTGCTAGGCGACCTGCAGCGGCTCCGGGTT
CTTACCCCTCAGCAGCGGGCGGTTGCCTTCTTTCG
AGCCAATACTAAGGAGCTAGAGGACTTCTTACGCT
CAGATGGGCAATCTGAGGAGATACTGTCTGGCCCC
CTCCTTAACCGTCTACTAGAACCCTCATGCCCTCT
TGATATTTTAACCGGATATCACCTATTTCGTCAGA
ATCCCAAGGCAGGTCAGTTGCGCGGCCTTGAGGTC
AAGATGCTTGAACGGTTATACGATGCTAATATTTA
CAATATATTGTCTCGGCTGCGACCTAAAAAAGTCC
GCAACAAGGCTATTGAGCTATACTGGGTTTTCCGA
GCTATCCATATTTGTCATGCTCCTTTAGTTTTAGA
TATTGTACGATATGAGGAACCGGACTTTGCTGAAC
TGGCCTTTATTTGTGCTGCTTACTTTGGTGAACCT
CAGGTAATGTATTTGCTCTACAAATATATGCCTCT
GACCCGCGCAGTTCTTACGGATGCCATCCAGATAA
GTCTTGAGAGCAACAACCAGGTAGGGATTTGCTAT
GCTTACTTGATGGGAGGCAGCCTCAAGGGACTAGT
CTCCGCCCCACTGCGTAAACGTCTGCGCGCCAAAC
TACGCTCGCAGCGCAAAAAGAAGGACGTTCTTTCA
CCCCACGACTTCTTACTGCTGCTCCAGTAG
Warthog DP148R
(AY261366.1:180836-181548)
SEQ ID No. 292
ATGTTAGAAATAGTATTGGCAACGCTGCTAGGTGA
CCTGCAGCGGCTCCGGGTTCTTACCCCTCAGCAGC
GGGCGGTTGCCTTCTTTCGAGCCAATACTAAGGAG
CTAGAGGACTTTTTATGCTCAGATGGGCAGTCTGA
GGAGATACTGTCCGGCCCCCTCCTTAACCGTCTAC
TAGAACCCTCAGGCCCTCTTGATATTTTAACCGGA
TATCACTTATTTCGTCAGAATCCCAAGGCAGGTCA
GGTGCGCGGCCTTGAGGTCAAGATGCTTGAACGGT
TATACGATGCTAATATTTACAATATATTGTCTCGG
CTGCGACCTGAAAAAGTTCGCAACAAGGCTGTTGA
GCTATACTGGGTTTTTCGGGCTATCAATATGTGTC
ATGCTCCTTTAGTTTTAGATATTGTACGATATGAG
GAACCGGACTTTGCTGAACTGGCCTTTATTTGTGC
TGCTTACTTTGGTGAACCTCAGGTAATGTATTTGC
TCTACAAATATATGCCTCTGACCCGCGCAGTTCTT
ACGGATGCCATCCAGATAAGTCTTGAGAGCAACAG
CCAGGTAGGGATTTGCTATGCCTACTTGATGGGAG
GCAGCCTCAAGGGCCTAGTCCGTGCCCCACTACGT
AAACGTCTGCGCGCCAAACTACGCTCGCAGCGCAA
AAAGAAGGACGTTCTTCCACCCCATGACTTCTTAC
TGCTGCTCCAGTAA
Pretorisuskop/96/4 DP148R
(AY261363.1:185149-185861)
SEQ ID No. 293
ATGTTAGAAATAGTATTGGCAACGCTGCTAGGTGA
CCTGCAGCGGCTCCGGGTTCTTACCCCTCAGCAGC
GGGCGGTTGCCTTCTTTCGAGCCAATACTAAGGAG
GTAGAAGACTTCTTATGCTCAGATGGGCAGTCTGA
GGAGGTACTGTCCGGCCCCCTCCTTAACCGTCTAC
TAGAACCCTCAGGCCCTCTTGATATTTTAACCGGA
TATCACTTATTTCGTCAGAATCCCAAGGCGGGTCA
GGTGCGCGGCCTTGAGGTCAAGATGCTTGAACGGT
TATACGATGCTAATATTTACAATATATTGTCTCGG
CTGCGACCTGAAAAAGTTCGCAACAAGGCTGTTGA
GCTATACTGGGTTTTTCGGGCTATCAATATGTGTC
ATGCTCCTTTAGTTTTAGATATTGTACGATATGAG
GAACCGGACTTTGCTGAACTGGCCTTTATTTGTGC
TGCTTACTTTGGTGAACCTCAGGTAATGTATTTGC
TCTACAAATATATGCCTCTGAGCCGCGCAGTTCTT
ACGGATGCCATCCAGATAAGTCTTGAGAGCAACAG
CCAGGTAGGGATTTGCTATGCCTACTTGATGGGAG
GCAGCCTCAAGGGCCTAGTCCGTGCCCCACTACGT
AAACGTCTGCGCGCCAAACTACGCTCGCAGCGCAA
AAAGAAGGACGTTCTTCCACCCCACGACTTCTTAC
TGCTGCTCCAGTAA
Tengani 62 DP148R
(AY261364.1:179845-180557)
SEQ ID No. 294
ATGTTAGAAATAGTATTGGCAACGCTGCTAGGTGA
CCTGCAGCGGCTCCGGGTTCTTACCCCTCAGCAGC
GGGCGGTTGCCTTCTTTCGAGCCAATACTAAAGAG
CTAGAGGACTTCTTATGTCCAGATGGGCAGTCTGA
GGAGGTGCTGTCTGGCTCCCTCCTTAACCGTCTAC
TAGAACCCTCAGGCCCTCTTGATATTTTAACTGGA
TATCACCTATTTCGTCAGAATCCCAAGGCAGGTCA
GTTGCGCGGCCTTGAGGTCAAGATGCTTGAACGGT
TATACGATGCTAATATTTACAACATATTGTCCCGG
CTGCGACCTGAAAAAGTTCGCAACAAGGCTGTTGA
GCTATACTGGGTTTTTCGGGCTATCAATATGTGTC
ATGCTCCTTTAGTTTTAGATATTGTACGAAATGAG
GAACTGGACTTTGCTGAACTGGCCTTTATTTGTGC
TGCTTACTTTGGTGAGCCTCAGGTAATGTATTTGC
TCTACAAATATATGCCTCTGACCCGCGCAGTTCTT
ACGGATGCCATCCAGATAAGTCTTGAGAGCAACAG
CCAGGTGGGGATTTGCTATGCCTACTTGATGGGAG
GCAGCCTCAAGGGACTAGTCCGTGCCCCGCTACGT
AAACGTCTGCGCGCCAAACTACGCTCGCAGCGCAA
AAAGAAGGATGTTCTTCCACCCCACGACTTCTTAC
TGCTGCTCCAGTAA
Ken06.Bus DP148R
(NC_044946.1:181097-181870)
SEQ ID No. 295
ATGCAAAATAAAATCCCAAATTTTAACCTTTTCTT
TTTTTTTCTATACAAGATGTTAGAAATAGTATTGG
CAACGCTGCTGGGTGATCTGCAGCAACTTAAGGAT
CTTACACCTAAGCAGCGGGCCGTAGCTTTCTTCCG
AGCCAACACCAAAGAGCTAGAGGACTTTCTCTACC
CCGATGGGCAGACTGAGGAGTTACTCCCTGGATTT
CTTCTTAACCATTTACTAGAACCCTCAGGCCCTAT
TGAAATTTTAACCGGATATCACCTCTTTCGTCAGA
ATCCAAAGGCAGGTCGCTTGCGAGGCCTTGAGGTG
AAAATGCTTGAACGGTTATATGATGCTAATATTTA
CAATATGCTGGCTCGGCTACGGCCTGAGTTGGTTC
GTGACAAGGCTGTTGAGCTATATTGGCTTTTTCGA
GCTATTTTAATATGTCATGGTCCCTTGGTTTTGGA
GATTGTACGACATGAGACGTTGGACTTTGCAGAAA
CCGCCTTTATCTGTGCTGCTTACTTTAGTGAACCT
CAGGTAATGTACGCTCTTTATAACTTTATACCACC
CACTCATGCAGTCCTTGCTGATGCCATCCAGATGT
GTCTTGAGAGCAACAGCGAGGCAGGGATTTGCTAT
GTCTACCTAATGGGAGGTAACCTCAAGGGCAAGGT
GCCCGGCTCGCTGCGCAAACGTCTGCGTGCCAGTC
CACTTCGGCAAGAACGCAAAAAGAAAAACGTCCTT
CCGCCCCACGAATTCCTACTCCTGCTCCACGGGAT
TTAA
Kenya 1950 DP148R+10
(AY261360.1:189099-189872)
SEQ ID No. 296
ATGCAAAATAAAATCCCAAATTTTAACCTTTTCTT
TTTTTTTCTATACAAGATGTTAGAAATAGTATTGG
CCACGCTGCTAGGTGATCTGCAGAAACTTAAGGAT
CTTACACCTCCGCAGCGGGCTGTGGCTTTCTTTCG
AGCCAACACCAAAGAGCTAGAGGACTTTCTCTACC
CCGATGGGCAGTCTGAGGAGTTACTCCCTGGACTT
CTCCTTAACCGTTTACTAGAACCTTCAGGTTCTAT
TGACATTTTAACCGGTTACCACCTATTTCGTGAGA
ATCCAAAGGCAGGTCGCTTGCGAGGCCTTGAGGTG
AAAATGCTTGAACGGTTATATGATGCTAATATTTA
CAATATGCTGGCTCGGCTACGGCCTGAGTTGGTTC
GTGACAAGGCTATTGAGCTATATTGGCTCTTTCGG
GCTATTTTAATGTGTCATAGTCCCTTAGTTTTGGA
GATTGTACGACATGAGACAATGGATTTTGCAGAAA
CCGCCTTTATCTGTGCCGCTTACTTTAGTGAACCT
CAGGTAATGTACGCTCTTTATAAATTTATACCTAT
CTCTCGTGCAGTCCTTGCTGATGCCATTCAGATGT
GTCTTGAGAGCAACAGCGAGGCGGGGATTTGCTAT
GCTTACCTAATGGGGGGTAGCCTCAAGGGCAAGGT
GCCTGGCTCGCTGCGCAAACGTCTGCGTGCCAGTC
CACTTCGGCAAGAGCGCAAAAAGAAAAACGTCCTC
CCGCCCCATGAATTCCTACTCATGCTCCATGGGAT
TTAA
Warmbaths DP148R
(AY261365.1:184288-185048)
SEQ ID No. 297
ATGCAAAATAAAATCCCAAATTTTAACCTTTTCTT
TTTTTTTCTATACAGGATGTTAGAAATAGTATTGG
CAACGCTGCTAGGTGACCTGCAGCGGCTCCGGGTT
CTTACCCCTCAGCAGCGGGCGGTTGCCTTCTTTCG
AGCCAATACTAAGGAGCTAGAGGACTTCTTATGCT
CAGATGGGCAGTCTGAGGAGATACTGTCCGGCCCC
CTCCTTAACCGTCTACTAGAACCCTCAGGCCCTCT
TGATATTTTAACCGGATATCACTTATTTCGTCAGA
ATCCCAAGGCAGGTCAGTTGCGCGGTCTTGAGGTC
AAGATGCTTGAACGGTTATACGATGCTAATATTTA
CAATATATTGTCTCGGCTGCGACCTGAAAAAGTTC
GCAACAAGGCTATTGAGCTATACTGGGTTTTCCGA
GCTATCCATATTTGTCATGCTCCTTTAGTTTTAGA
TATTGTACGATATGAGGAACCGGACTTTGCTGAAC
TGGCCTTTATTTGTGCTGCTTACTTTGGTGAACCT
CAGGTAATGTATTTGCTCTACAAATATATGCCTCT
GACCCGCGCAGTTCTTACGGATGCCATCCGGATAA
GTCTTGAGAGTAACAACCAGGTAGGGATTTGCTAT
GCTTACTTGATGGGAGGCAGCCTCAAGGGACTAGT
CTCCGCCCCACTGCGTAAACGTCTGCGCGCCAAAC
TACGCTCACAGCGCAAAAAGAAAGACGTTCTTTCA
CCCCACGACTTCTTACTGCTGCTCCAGTAA
Mkuzi 1979 DP148R
(AY261362.1:185751-186511)
SEQ ID No. 298
ATGCAAAATAAAATCCCAAATTTTAACCTTTTCTT
TTTTTTTCTATACAGGATGTTAGAAATAGTATTGG
CAACGCTGCTAGGCGACCTGCAGCGGCTCCGGGTT
CTTACCCCTCAGCAGCGGGCGGTTGCCTTCTTTCG
AGCCAATACTAAGGAGCTAGAGGACTTCTTATGCT
CAGATGGGCAGTCTGAGGAGATACTATCTGGCCCC
CTTCTTAACCGTCTACTAGAACCCTCAGGCCCTCT
TGATATTTTAACTGGATATCACCTATTTCGTCAGA
ACCCCAAGGCAGGTCAGTTACGCGGCCTTGAGGTC
AAGATGCTTGAACGGTTATACGATGCTAATATTTA
CAATATATTGTCTCGGCTGCGACCTGAAAAAGTTC
GCAACAAGGCTATTGAGCTTTACTGGGTTTTTCGA
GCTATCCATATTTGTCATGCTCCTTTAGTTTTAGA
TATTGTACGATATGAGGAACCGGACTTTGCTGAAC
TGGCCTTTATTTGTGCTGCTTACTTTGGTGAACCT
CAGGTAATGTATTTGCTCTACAAATATATGCCTCT
GACCCGCGCAGTTCTTACGGATGCCATCCGGATAA
GTCTTGAGAGTAACAACCAGGTAGGGATTTGCTAT
GCTTACTTGATGGGAGGCAGCCTCAAGGGACTAGT
CTCCGCCCCACTGCGTAAACGTCTGTGCGCCAAAC
TACGCTCGCAGCGCAAAAAGAAGGACGTTCTTTCA
CCCCACGACTTCTTACTGCTGCTCCAGTAA
Ken05/Tk1 DP148R
(NC_044945.1:186074-186847)
SEQ ID No. 299
ATGCAAAATAAAATCCCAAATTTTAACCTTTTCTT
TTTTTTTCTATACAAGATGTTAGAAATAGTATTGG
CAACGCTGCTAGGTGATCTGCAGAAACTTAAAGAT
CTTACACCTATGCAGCGGGCTGTGGCTTTCTTTCG
AGCCAACACTAAAGAGCTAGAGGACTTCCTCTGCC
CCGATGGGCAGTCTGAGGAGTTACTCCCTGGACTT
CTCCTTAACCGTTTATTAGAACCTTCAGGTCCTAT
TGACATTTTAACCGGTTACCACCTATTTCGTGAGA
ATCCGAAGGCAGGTCGGCTGCGTGGCCTTGAGGTC
AAGCTGCTTGAACGGTTATATGATGCTAACATCTA
CAATATATTGGCCCAGCTACGGCCTGAGTTGGTTC
GCAATAAGGCTGTTGAGTTATATTGGCTCTTTCGG
GCTATTTTGATGTGTCATGGTCCCTTAGTTTTGGA
GATTGTACGACATGAGACAATGGATTTTGCAGAAT
TAGCCTTTATCTGTGCTGCTTACTTTAGTGAACCT
CAGGTAATGTACGCTATTTATAAATTTATACCTAT
CTCTTGCGCAGTCCTTGCTGATGCCATTCAGATGT
GTCTTGAGAGTAACAGCGAGGCGGGGATTTGCTAT
GCTTACCTAATGGGGGGTAGCCTCAAGGGCAAGGT
GCCTGGCGCGCTGCGCAAACGTCTGCGTGCCAGTC
CACTTCGGCAAGAGCGCAAAAAGAAAAACGTTCTT
CCACCCCATGAATTCCTACTCATGCTTCATGGGAT
TTAA
Malawi Lil-20/1 (1983) DP148R
(AY261361.1:183573-184346)
SEQ ID No. 300
ATGTTAGAAATAGTATTGGCAACGCTGCTAGGTGA
TCTGCAGAAGCTTAGGGATCTTACACCTATGCAGC
GGGCTGTGGCTTTCTTTCGAGTCAACACTAAAGAG
CTAGAGGACTTCCTCTACCCCGATGGGCAGTCTGA
GGAGTTGCTCCCTGGACTTCTCCTTAACCGTTTAC
TAGAACCTTCAGGCCCTATTGACATTTTAACCGGT
TACCACCTATTTCGTGAGAACCCAAAGGCAGGTCG
GCTGCGTGGCCTTGAGGTTAAGCTGCTTGAACGGT
TATATGATGCTAACATCTACAATATGTTGGCCCAA
ATACGACCTGAGTTGGTTCGTATCAAGGCTATTGA
GTTATATTGGCTCTTTCGGGCTATTTTAATGTGTC
ATAGTCCCTTAGTTTTGGAGATTGTACGACATGAG
ACAATGGATTTTGCAGAATTAGCCTTTATCTGTGC
TGCTTACTTTAGTGAACCTCAGGTAATGTACGCTC
TTTATAAATTTATACCTATTTCTCGCGCAGTCCTT
GCTGATGCCATCGAGATGTCTCTTGAGAGCAACAG
CGAGACGGGGATTTGCTATGCCTACCTAATGGGGG
GTAGCCTCAAGGGCAAGGTGCCCGGCCCGCTGCGC
AAACGTCTGCGTGCCAGTCCACTTCGGCAAGAGCG
CAAAAAGAAAAACGTCCTTCCGCCCCATGAATTTC
TACTCATGCTCCATGGGATTTAA

In an embodiment the invention provides an attenuated ASF virus in which the expression and/or activity of the DP148R gene is disrupted. Suitably, the DP148R gene comprises the sequence of SEQ ID No. 287, 288, 289, 290, 291, 292, 293, 294, 295, 296, 297, 298, 299 or 300. Suitably, the DP148R gene comprises a sequence having at least 70%, at least 80%, at least 90% or at least 95% identity with SEQ ID No. 287, 288, 289, 290, 291, 292, 293, 294, 50 295, 296, 297, 298, 299 or 300. Suitably, the DP148R gene consists of the sequence of SEQ ID No. 287, 288, 289, 290, 291, 292, 293, 294, 295, 296, 297, 298, 299 or 300.

The amino acid sequence of the Benin 97/1 DP148R protein is depicted below as SEQ ID No. 11:

SEQ ID No. 11
Benin 97/1 DP148R protein
MQNKIPNFNLFFFFLYRMLEIVLATLLGDLQRLRV
LTPQQRAVAFFRANTKELEDFLRSDGQSEEILSGP
LLNRLLEPSCPLDILTGYHLFRQNPKAGQLRGLEV
KMLERLYDANIYNILSRLRPKKVRNKAIELYWVFR
AIHICHAPLVLDIVRYEEPDFAELAFICAAYFGEP
QVMYLLYKYMPLTRAVLTDAIQISLESNNQVGICY
AYLMGGSLKGLVSAPLRKRLRAKLRSQRKKKDVLS
PHDFLLLLQ

Suitably, the DP148R gene encodes a protein comprising the sequence of SEQ ID No. 11. Suitably, the DP148R gene encodes a protein comprising a sequence having at least 70%, at least 80%, at least 90% or at least 95% identity with SEQ ID No. 11. Suitably, the DP148R gene encodes a protein consisting of the sequence of SEQ ID No. 11.

Orthologous DP148R sequences from other genomes share between 74 and 99% amino acid identity. The orthologous DP148R genes from other ASFV isolates are depicted below as SEQ ID Nos 12 to 19, and their location in the ASFV genome is indicated:

Warthog DP148R protein:
181103 to 181549
SEQ ID No. 12
MLEIVLATLLGDLQRLRVLTPQQRAVAFFRANTKE
LEDFLCSDGQSEEILSGPLLNRLLEPSGPLDILTG
YHLFRQNPKAGQVRGLEVKMLERLYDANIYNILSR
LRPEKVRNKAVELYWVFRAINMCHAPLVLDIVRYE
EPDFAELAFICAAYFGEPQVMYLLYKYMPLTRAVL
TDAIQISLESNSQVGICYAYLMGGSLKGLVRAPLR
KRLRAKLRSQRKKKDVLPPHDFLLLLQ
Kenya 1950 DP148R protein:
189417 to 189872
SEQ ID No. 13
MQNKIPNFNLFFFFLYKMLEIVLATLLGDLQKLKD
LTPPQRAVAFFRANTKELEDFLYPDGQSEELLPGL
LLNRLLEPSGSIDILTGYHLFRENPKAGRLRGLEV
KMLERLYDANIYNMLARLRPELVRDKAIELYWLFR
AILMCHSPLVLEIVRHETMDFAETAFICAAYFSEP
QVMYALYKFIPISRAVLADAIQMCLESNSEAGICY
AYLMGGSLKGKVPGSLRKRLRASPLRQERKKKNVL
PPHEFLLMLHGI
Malawi LIL20/1 DP148R protein:
183687 to 184346
SEQ ID No. 14
MLEIVLATLLGDLQKLRDLTPMQRAVAFFRVNTKE
LEDFLYPDGQSEELLPGLLLNRLLEPSGPIDILTG
YHLFRENPKAGRLRGLEVKLLERLYDANIYNMLAQ
IRPELVRIKAIELYWLFRAILMCHSPLVLEIVRHE
TMDFAELAFICAAYFSEPQVMYALYKFIPISRAVL
ADAIEMSLESNSETGICYAYLMGGSLKGKVPGPLR
KRLRASPLRQERKKKNVLPPHEFLLMLHGI
Mkuzi DP148R protein:
185751 to 186515
SEQ ID No. 15
MQNKIPNFNLFFFFLYRMLEIVLATLLGDLQRLRV
LTPQQRAVAFFRANTKELEDFLCSDGQSEEILSGP
LLNRLLEPSGPLDILTGYHLFRQNPKAGQLRGLEV
KMLERLYDANIYNILSRLRPEKVRNKAIELYWVFR
AIHICHAPLVLDIVRYEEPDFAELAFICAAYFGEP
QVMYLLYKYMPLTRAVLTDAIRISLESNNQVGICY
AYLMGGSLKGLVSAPLRKRLCAKLRSQRKKKDVLS
PHDFLLLLQ
Pretorisuskop DP148R protein:
185416 to 185862
SEQ ID No. 16
MLEIVLATLLGDLQRLRVLTPQQRAVAFFRANTKE
VEDFLCSDGQSEEVLSGPLLNRLLEPSGPLDILTG
YHLFRQNPKAGQVRGLEVKMLERLYDANIYNILSR
LRPEKVRNKAVELYWVFRAINMCHAPLVLDIVRYE
EPDFAELAFICAAYFGEPQVMYLLYKYMPLSRAVL
TDAIQISLESNSQVGICYAYLMGGSLKGLVRAPLR
KRLRAKLRSQRKKKDVLPPHDFLLLLQ
Tengani62 DP148R protein:
180112 to 180558
SEQ ID No. 17
MLEIVLATLLGDLQRLRVLTPQQRAVAFFRANTKE
LEDFLCPDGQSEEVLSGSLLNRLLEPSGPLDILTG
YHLFRQNPKAGQLRGLEVKMLERLYDANIYNILSR
LRPEKVRNKAVELYWVFRAINMCHAPLVLDIVRNE
ELDFAELAFICAAYFGEPQVMYLLYKYMPLTRAVL
TDAIQISLESNSQVGICYAYLMGGSLKGLVRAPLR
KRLRAKLRSQRKKKDVLPPHDFLLLLQ
Warmbaths DP148R protein:
184606 to 185052
SEQ ID No. 18
MQNKIPNFNLFFFFLYRMLEIVLATLLGDLQRLRV
LTPQQRAVAFFRANTKELEDFLCSDGQSEEILSGP
LLNRLLEPSGPLDILTGYHLFRQNPKAGQLRGLEV
KMLERLYDANIYNILSRLRPEKVRNKAIELYWVFR
AIHICHAPLVLDIVRYEEPDFAELAFICAAYFGEP
QVMYLLYKYMPLTRAVLTDAIRISLESNNQVGICY
AYLMGGSLKGLVSAPLRKRLRAKLRSQRKKKDVLS
PHDFLLLLQ
OURT88/3 DP148R protein:
169146 to 169592
SEQ ID No. 19
MQNKIPNFNLFFFFLYRMLEIVLATLLGDLQRLRVLT
PQQRAVAFFRANTKELEDFLRSDGQSEEILSGPPP
China/2018/Anhui DP148R protein
SEQ ID No. 301
MLEIVLATLLGDLQRLRVLTPQQRAVAFFRANTKE
LEDFLCSDGQSEEVLSGPLLNRLLEPSGPLDILTG
YHLFRQNPKAGQLRGLEVKMLERLYDANIYNILSR
LRPEKVRNKAIELYWVFRAIHICHAPLVLDIVRYE
EPDFAELAFICAAYFGEPQVMYLLYKYMPLTRAVL
TDAIRISLESNNQVGICYAYLMGGSLKGLVSAPLR
KRLRAKLRSQRKKKDVLSPHDFLLLLQ
Georgia2007/1 DP148R protein
SEQ ID No. 302
MLEIVLATLLGDLQRLRVLTPQQRAVAFFRANTKE
LEDFLCSDGQSEEVLSGPLLNRLLEPSGPLDILTG
YHLFRQNPKAGQLRGLEVKMLERLYDANIYNILSR
LRPEKVRNKAIELYWVFRAIHICHAPLVLDIVRYE
EPDFAELAFICAAYFGEPQVMYLLYKYMPLTRAVL
TDAIRISLESNNQVGICYAYLMGGSLKGLVSAPLR
KRLRAKLRSQRKKKDVLSPHDFLLLLQ
Ken05/Tk1 DP148R protein
SEQ ID No. 303
MQNKIPNFNLFFFFLYKMLEIVLATLLGDLQKLKD
LTPMQRAVAFFRANTKELEDFLCPDGQSEELLPGL
LLNRLLEPSGPIDILTGYHLFRENPKAGRLRGLEV
KLLERLYDANIYNILAQLRPELVRNKAVELYWLFR
AILMCHGPLVLEIVRHETMDFAELAFICAAYFSEP
QVMYAIYKFIPISCAVLADAIQMCLESNSEAGICY
AYLMGGSLKGKVPGALRKRLRASPLRQERKKKNVL
PPHEFLLMLHGI
Ken06 DP148R protein
SEQ ID No. 304
MQNKIPNFNLFFFFLYKMLEIVLATLLGDLQQLKD
LTPKQRAVAFFRANTKELEDFLYPDGQTEELLPGF
LLNHLLEPSGPIEILTGYHLFRQNPKAGRLRGLEV
KMLERLYDANIYNMLARLRPELVRDKAVELYWLFR
AILICHGPLVLEIVRHETLDFAETAFICAAYFSEP
QVMYALYNFIPPTHAVLADAIQMCLESNSEAGICY
VYLMGGNLKGKVPGSLRKRLRASPLRQERKKKNVL
PPHEFLLLLHGI
L60 DP148R protein
SEQ ID No. 305
MQNKIPNFNLFFFFLYRMLEIVLATLLGDLQRLRV
LTPQQRAVAFFRANTKELEDFLRSDGQSEEILSGP
LLNRLLEPSCPLDILTGYHLFRQNPKAGQLRGLEV
KMLERLYDANIYNILSRLRPEKVRNKAIELYWVFR
AIHICHAPLVLDIVRYEEPDFAELAFICAAYFGEP
QVMYLLYKYMPLTRAVLTDAIQISLESNNQVGICY
AYLMGGSLKGLVSAPLRKRLRAKLRSQRKKKDVLS
PHDFLLLLQ

Suitably, the DP148R gene encodes a protein comprising the sequence of SEQ ID No. 12.Suitably, the DP148R gene encodes a protein comprising the sequence of SEQ ID No. 13. Suitably, the DP148R gene encodes a protein comprising the sequence of SEQ ID No. 14. Suitably, the DP148R gene encodes a protein comprising the sequence of SEQ ID No. 15. Suitably, the DP148R gene encodes a protein comprising the sequence of SEQ ID No. 16. Suitably, the DP148R gene encodes a protein comprising the sequence of SEQ ID No. 17. Suitably, the DP148R gene encodes a protein comprising the sequence of SEQ ID No. 18. Suitably, the DP148R gene encodes a protein comprising the sequence of SEQ ID No. 19. Suitably, the DP148R gene encodes a protein comprising the sequence of SEQ ID No. 301. Suitably, the DP148R gene encodes a protein comprising the sequence of SEQ ID No. 302. Suitably, the DP148R gene encodes a protein comprising the sequence of SEQ ID No. 303. Suitably, the DP148R gene encodes a protein comprising the sequence of SEQ ID No. 304. Suitably, the DP148R gene encodes a protein comprising the sequence of SEQ ID No. 305.

### EP153R

The EP153R gene is expressed both early and late in infection. EP153R may also be referred to as 8CR. The EP153R protein is a C-type lectin. C-type animal lectins are found in serum, the extracellular matrix and cell membranes and are thought to act as receptors for carbohydrate ligands. The EP153R protein comprises a C-type lectin domain, a cell attachment sequence (RGD) and a transmembrane domain, and has similarity with CD44 molecules involved in T cell activation.

The gene (i.e. nucleotide) sequences and positions in the genome of EP153R genes from different ASFV strains are presented below.

Benin 97/1 EP153R
(NC_044956.1:67030-67491)
SEQ ID No. 204
ATGTATTTTAAGAAAAAATACATCGGTCTTATTGA
TAAGAACTGTGAAAAAAAAATATTAGATGATTCTA
GTACAATAAAAATTTGTTACATATTAATTGGAATA
TTGATTGGAACTAATATGATAACTCTTATTTATAA
TTTCATATTCTGGGATAATTATATAAAATGTTACC
GAAATAATGATAAAATGTTTTACTGTCCTAATGAT
TGGGTTGGATATAATAATATTTGTTACTATTTTAG
TAATGGTAGTTTTTCTAAAAATTATACAGCTGCTA
GTAATTTTTGTAGACAATTAAATGGTACACTTGCT
AATAATGATACTAATTTATTAAATCTAACTAAAAT
ATATAATAATCAATCTATGTATTGGGTTAACAATA
CGGTAATATTACGTGGTGATAATAAATATAGTCAA
AAAGTTAACTATACAGATTTATTATTTATTTGTGG
TAAATAA
Georgia 2007/1 EP153R
(NC_044959.1:73369-74451)
SEQ ID No. 205
ATGTTTTCTAACAAAAAGTACATCGGTCTTATCAA
TAAGAAGGAGGGTTTGAAAAAAAAAATAGATGATT
ATAGTATATTAATAATTGGAATATTAATTGGAACT
AACATCTTAAGCCTTATTATAAATATAATAGGAGA
GATTAATAAACCAATATGTTACCAAAATGATGATA
AGATATTTTATTGCCCTAAAGATTGGGTTGGATAT
AATAATGTTTGTTATTATTTTGGCAATGAAGAAAA
AAATTATAATAATGCAAGTAATTATTGTAAGCAAT
TAAATAGTACGCTTACTAATAATAATACTATTTTA
GTAAATCTTACTAAAACATTAAATCTTACTAAAAC
ATATAATCACGAATCTAATTATTGGGTTAATTATT
CTTTAATTAAAAATGAGTCAGTACTATTACGTGAT
AGTGGATATTACAAAAAACAAAAACATGTAAGTTT
ATTATATATTTGTAGTAAATAA
Ken05/Tk1 EP153R
(NC_044945.1:73544-74038)
SEQ ID No. 206
ATGTATTCTAAGAAAAAGTACATCGGTCTTATTGA
CAAGAACTGTGAAAAAAAAATATTAGATGATGCTA
CTACAATAAAAATTTGTTACATATTAATTGGAATA
TTGATTGGAACTAATATTATAACTCTTATCTATAA
TTTCATATTTTGGGAGAATTATATAAAATGTATCC
AAAATGATGATAAGATGTTTTACTGTCCTAAAGAT
TGGGTTGGGTATAATAATGCGTGTTATTATTTTAG
TAATAACAATAAAAATTATACAGATGCAAATAATT
ATTGTAAAAATTCACATAATAGTACACTTGCTAAT
AATGATACTAAATTATTAAATCTTACTAAATTATT
AAATCTTTCTAAATTATATTACAATGATTCTACAT
ATTGGGTTAAATATTCTTTACCTAAAAATAAAGCA
GTAACATTACGTAACAGTACCTACAAATATGATAG
AGTTAAATCTACAGAAACATTTTTTATTTGTAGTA
ACTAA
Ken06.Bus EP153R
(NC_044946.1:69244-69741)
SEQ ID No. 207
ATGTATTCTAAGAAAAAGTACATCGGTCTTATTGA
CAAAAGCTGTGAAAAAAAAATATTAGATGATCCTA
CTACAATAAAAATTTGTTACATATTAATTGGAATA
TTGATTGGAACTAATATTATAACTCTTATCTATAA
TTTCATATTTTGGGAGAATTATATAAAATGTATCC
GAAATGATGATAAGATGTTTTACTGCCCTAAAGAT
TGGGTTGGATATAATAATGTGTGTTATTATTTTAG
TAACGATAATGGAAAAAATTATATGAATGCAAGTA
ATTTTTGTAATAAAAAAAATAGCACGCTTGCTAAT
AATACGACTAATTTAGTAAATCTTACTAAAATATT
AAATTTTACCAAACAATATAATCATGAATCACTTT
ATTGGGTTAATTATTCTTTAAGTAATAATATGTCA
TTAGCATTACAGAATAGTAATTTTCCTTCTTCAGG
AAGACGTAAATATATGGACTTATTATATATATGTA
GTATATAA
L60 EP153R (NC_044941.1:67379-67840)
SEQ ID No. 208
ATGTATTTTAAGAAAAAATACATCGGTCTTATTGA
TAAGAACTGTGAAAAAAAAATATTAGATGATTCTA
GTACAATAAAAATTTGTTACATATTAATTGGAATA
TTGATTGGAACTAATATGATAACTCTTATTTATAA
TTTCATATTCTGGGATAATTATATAAAATGTTACC
GAAATAATGATAAAATGTTTTACTGTCCTAATGAT
TGGGTTGGATATAATAATATTTGTTACTATTTTAG
TAATGGTAGTTTTTCTAAAAATTATACAGCTGCTA
GTAATTTTTGTAGACAATTAAATGGTACACTTGCT
AATAATGATACTAATTTATTAAATCTAACTAAAAT
ATATAATAATCAATCTATGTATTGGGTTAACAATA
CGGTAATATTACGTGGTGATAATAAATATAGTCAA
AAAGTTAACTATACAGATTTATTATTTATTTGTGG
TAAATAA
China/2018/AnhuiXCGQ EP153R
(MK128995.1:72837-73313)
SEQ ID No. 209
ATGTTTTCTAACAAAAAGTACATCGGTCTTATCAA
TAAGAAGGAGGGTTTGAAAAAAAAAATAGATGATT
ATAGTATATTAATAATTGGAATATTAATTGGAACT
AACATCTTAAGCCTTATTATAAATATAATAGGAGA
GATTAATAAACCAATATGTTACCAAAATGATGATA
AGATATTTTATTGCCCTAAAGATTGGGTTGGATAT
AATAATGTTTGTTATTATTTTGGCAATGAAGAAAA
AAATTATAATAATGCAAGTAATTATTGTAAGCAAT
TAAATAGTACGCTTACTAATAATAATACTATTTTA
GTAAATCTTACTAAAACATTAAATCTTACTAAAAC
ATATAATCACGAATCTAATTATTGGGTTAATTATT
CTTTAATTAAAAATGAGTCAGTACTATTACGTGAT
AGTGGATATTACAAAAAACAAAAACATGTAAGTTT
ATTATATATTTGTAGTAAATAA
Malawi Lil-20/1 (1983) EP153R
SEQ ID No. 210
(AY261361.1:70776-71261)
ATGTTTTCTAACAAAAAGTACATCGGTCTTATTGA
CAAGTACTGTGAAAAAAAAATATTAGATGATTCTA
GTACAATAAAAATTTGTTACATATTAATTGGAATA
CTGATTGGAACTAATATGATAACTCTTATTTATAA
TTTCATATTTTGGGAGAATTATATAACATGTAACC
AAAAAGATAAGACGTTTTACTGTCCTAAAGATTGG
GTTGGATATAATAATGTTTGTTATTATTTTGGCAA
TGATGAAAAAAATTATAATAATGCAAGTAATTATT
GTAAGCAATTAAATAGTACGCTTACTAATAATAAT
ACTAATTTAGTAAATCTTACTAAAACATTAAATCT
TACTAAAACATATAATCACGAATCTAATTATTGGG
TTAATTATTCTTTAATTAAAAATGAGTCAGTACTA
TTACGCAATAGTGGATATTATAAAAAACAAAAACA
TGTAAGTTTATTATATATTTGTAGTAAATAA
Warthog EP153R (AY261366.1:69453-69944)
SEQ ID No. 211
ATGTTTTCTAATAAAAAGTACATTGGTCTTATCAA
TAAGAAGGAGGGATTGAAAAAAAAAATAGATGATT
ATAGTATATTAATAATTGGAATATTAATTGGAACT
AACATCTTAAGCCTTATTATAAATATAATAGGAGA
GATTAATAAACCAATATGTTACCAAAATAATGATA
AGATATTTTATTGCCCTAAAGATTGGGTTGGATAT
AATAATGTTTGTTATTATTTTAGTAATGATAATGG
CAATAATTATACAACTGCGGATAATAAATGTAAAC
AATTAAATAATAGTACACTGGCTAATAATCTTACT
GATTTATTAAATCTTACGAGTTTCTTAAATCTTAC
TAAATTATATCATCATCATTCTCATTATTGGGTTA
ATTATTCTTTAAATAATAATTATTCAGTACCATTA
ATTGATAGTAAATATAATTTAAATAGGAAAAAAAG
TCACTATACAGATTTACTATTTATTTGCAGCAAAT
AA
Mkuzi 1979 EP153R (AY261362.1:74549-75013)
SEQ ID No. 212
ATGTATTTTAAGAAAAAATACATCGGTCTTATTGA
TAAGAACTGTGAAAAAAAAATATTAGATGATTCTA
GTACAATAAAAATTTGTTACATATTAATTGGAATA
TTGATTGGAACTAATATGATAACTCTTATTTATAA
TTTCATATTCTGGGATAATTATATAAAATGTTACC
GAAATAATGATAAAATGTTTTACTGTCCTAATGAT
TGGGTTGGATATAATAATGTTTGTTACTATTTTAG
TAATGGTAGTTTTTTTAAAAATTATACAGATGCTA
ATAATTATTGTAAAAAAATAAATAGTACTCTTGCT
AATAATGATACTAATTTATTAAGTTTAACTAAAAT
ATACAATAATCAATCTATGTATTGGGTTAACAATA
CAGTAGCATTACGTGGTGATAGTGTATATGCACAA
AGAAAAGTTAATAATTCAGATCTATTATTTATTTG
TAGTAAATAA
Warmbaths
EP153R (AY261365.1:72727-73191)
SEQ ID No. 213
ATGTATTTTAAGAAAAAGTACATCGGTCTTATTGA
TAAGAACTGTGAAAAAAAAATATTAGATGATTGTA
CTACAATAAAAATTTGTTACATATTAATTGGAATA
TTGATTGGAACTAATATGATAACTCTTATTTATAA
TTTCATATTTTGGGATCATTATATGACATGTAACA
AAAAAGATAAGATGTTTTACTGTCCTAAAGATTGG
GTTGGATATAATAATGTTTGTTATTATTTTAGTAA
TGATAGTTTTACTAAGAATTATACATATGCAAGTA
ATTCTTGTAAAAAAATAAATAGTACACTTGCTAAT
AATGATACTAATTTACTAAATCTTATTAAAGTATA
TAATAATGTATCTGAGTATTGGGTTAATAATACAG
TAACATTACGTGGTGATCATAAATACATAAATAAG
CAAAATAATTGTAATACAGAATTTTTATTTATTTG
TAGTAGATAG
Tengani 62 EP153R
(AY261364.1:68134-68604)
SEQ ID No. 214
ATGTTTTCTAACAAAAAGTATCCGAGTCTTATCGA
AAAAAAAATGGATGACTTGATGACTTTAAAGTTTT
GTTACATCATTATTGCAATCTTGATCATGACTAAT
ATTTTTAGCCTTGTTGTGAATATATGGGGGGGGGG
TGATCGACAATCTTATGAGAATGTATTTTATTGTC
CTAAAGATTGGGTTGGATATAATAATGCATGTTAT
TATTTTAGTAATGATATGAAAAATTATACAGAAGC
AAGTAATTATTGTAAAAATTTATATAATAGTACAA
TTGTTAATAATAATACTAATATAGTAAATCTTACT
AAAACATTAAATCTTACTAAAATATATAATCATGA
AAGTAATTATTGGGTTAATTATTCTTTAATTAATA
ATGAGTCATTAACATTACGGGACAGTAATTTTCCT
TCTTCAGGAAAACGTACATATATAGATTTATTATA
TATATGTAGTATATAA
Pretorisuskop/96/4 EP153R
(AY261363.1:73562-74029)
SEQ ID No. 215
ATGTATTTTAAGAAAAAGTACATCGGTCTTATTGA
TAAGAACTGTGAAAAAAAAATATTAGATGATTGTA
CTACAATAAAAATTTGTTACATATTAATTGGAATA
TTGATTGGAACTAATATGATAACTCTTATTTATAA
TTTCATATTTTGGGATCATTATATGACATGTAACA
AAAAAGATAAGATGTTTTACTGTCCTAAAGATTGG
GTTGGATATAATAATGTTTGTTATTATTTTAATAA
TGATAGTAAGAATTATACAACTGCTACTAATTCTT
GTAAACAATTAAATAGTACACTTGCTAATAATGAT
ACTAATTTATTAAATCTTACTAAAGTATATCACCA
TGATAAATTATATTGGGTTAACTATTCTTTAAATG
ACAATTTTTCATTATCATTACGTAATAGTACATAT
GAAAAAAGATCTAAATATTTGCCATTATTATTTAT
TTGCAGTAAATAA
Kenya 1950 EP153R
(AY261360.1:76255-76731)
SEQ ID No. 216
ATGTTTTTGAACAAAAAGTATCCGAGTCTTATCGA
AAAAAAAATGGATGATTTGATGACTTTAAAGTTTT
GTTACTTAATAATCACATTTTTAATCATAACCAAT
ATTTTTAGCCTTGCTATAAATATATGGGGGGGGGG
TGATATGATAGATCGACAAAGTTGTGAAAATATAT
TTTACTGTCCTAAAGATTGGGTTGGCTATAATAAT
GCGTGTTATTATTTTAGTAATAACAATAAAAATTA
TACAGATGCAAATAATTATTGTAAAAATTCACATA
ATAGTACACTTGCTAATAATGATACTAAATTATTA
AATCTTACTAAATTATTAAATCTTTCTAAATTATA
TTACAATGATTCTACATATTGGGTTAAATATTCTT
TACCTAAAAATAAAGCAGTAACATTACGTAACAGT
ACCTACAAATATGATAGAGTTAAATATACAGAAAC
ATTTTTTATTTGTAGTAACTAA

In an embodiment the invention provides an attenuated ASF virus in which the expression and/or activity of the EP153R gene is disrupted. Suitably, the EP153R gene comprises the sequence of SEQ ID No. 204, 205, 206, 207, 208, 209, 210, 211, 212, 213, 214, 215 or 216. Suitably, the EP153R gene comprises a sequence having at least 70%, at least 80%, at least 90% or at least 95% identity with SEQ ID No. 204, 205, 206, 207, 208, 209, 210, 211, 212, 213, 214, 215 or 216. Suitably, the EP153R gene consists of the sequence of SEQ ID No. 204, 205, 206, 207, 208, 209, 210, 211, 212, 213, 214, 215 or 216.

The accession numbers of EP153R proteins from different strains of ASFV are listed below in Table 3.

TABLE 3

| | EP153R protein |
|---|---|
| Strain | accession number |
| BA71V | Q65150 |
| Benin 97/1 | A9L06 |
| E75 | D4I5P5 |
| Georgia 2007/1 | E0WMJ5 |
| Kenya | P0CA63 |
| Malawi | P0CA64 |
| Portugal/OURT88/1988 | A9JLS3 |
| PretorisuskopPr4/1996 | O89335 |
| Warthog | P0CA65 |

The amino acid sequences of the EP153R proteins from different ASFV strains are depicted below.

Benin 97/1 EP153R protein
SEQ ID No. 20
MYFKKKYIGLIDKNCEKKILDDSSTIKICYILIGI
LIGTNMITLIYNFIFWDNYIKCYRNNDKMFYCPND
WVGYNNICYYFSNGSFSKNYTAASNFCRQLNGTLA
NNDTNLLNLTKIYNNQSMYWVNNTVILRGDNKYSQ
KVNYTDLLFICGK
Georgia 2007/1 EP153R protein
SEQ ID No. 217
MFSNKKYIGLINKKEGLKKKIDDYSILIIGILIGT
NILSLIINIIGEINKPICYQNDDKIEYCPKDWVGY
NNVCYYFGNEEKNYNNASNYCKQLNSTLTNNNTIL
VNLTKTLNLTKTYNHESNYWVNYSLIKNESVLLRD
SGYYKKQKHVSLLYICSK*
Ken05/Tk1 EP153R protein
SEQ ID No. 218
MYSKKKYIGLIDKNCEKKILDDATTIKICYILIGI
LIGTNIITLIYNFIFWENYIKCIQNDDKMFYCPKD
WVGYNNACYYFSNNNKNYTDANNYCKNSHNSTLAN
NDTKLLNLTKLLNLSKLYYNDSTYWVKYSLPKNKA
VTLRNSTYKYDRVKSTETFFICSN*
KenO6.Bus EP153R protein
SEQ ID No. 219
MYSKKKYIGLIDKSCEKKILDDPTTIKICYILIGI
LIGTNIITLIYNFIFWENYIKCIRNDDKMFYCPKD
WVGYNNVCYYFSNDNGKNYMNASNFCNKKNSTLAN
NTTNLVNLTKILNFTKQYNHESLYWVNYSLSNNMS
LALQNSNFPSSGRRKYMDLLYICSI*
L60 EP153R protein
SEQ ID No. 220
MYFKKKYIGLIDKNCEKKILDDSSTIKICYILIGI
LIGTNMITLIYNFIFWDNYIKCYRNNDKMFYCPND
WVGYNNICYYFSNGSFSKNYTAASNFCRQLNGTLA
NNDTNLLNLTKIYNNQSMYWVNNTVILRGDNKYSQ
KVNYTDLLFICGK*
China/2018/AnhuiXCGQ EP153R protein
SEQ ID No. 221
MFSNKKYIGLINKKEGLKKKIDDYSILIIGILIGT
NILSLIINIIGEINKPICYQNDDKIFYCPKDWVGY
NNVCYYFGNEEKNYNNASNYCKQLNSTLTNNNTIL
VNLTKTLNLTKTYNHESNYWVNYSLIKNESVLLRD
SGYYKKQKHVSLLYICSK*
Malawi Lil-20/1 (1983) EP153R protein
SEQ ID No. 222
MFSNKKYIGLIDKYCEKKILDDSSTIKICYILIGI
LIGTNMITLIYNFIFWENYITCNQKDKTFYCPKDW
VGYNNVCYYFGNDEKNYNNASNYCKQLNSTLTNNN
TNLVNLTKTLNLTKTYNHESNYWVNYSLIKNESVL
LRNSGYYKKQKHVSLLYICSK*
Warthog EP153R protein
SEQ ID No. 223
MFSNKKYIGLINKKEGLKKKIDDYSILIIGILIGT
NILSLIINIIGEINKPICYQNNDKIFYCPKDWVGY
NNVCYYFSNDNGNNYTTADNKCKQLNNSTLANNLT
DLLNLTSFLNLTKLYHHHSHYWVNYSLNNNYSVPL
IDSKYNLNRKKSHYTDLLFICSK*
Mkuzi 1979 EP153R protein
SEQ ID No. 224
MYFKKKYIGLIDKNCEKKILDDSSTIKICYILIGI
LIGTNMITLIYNFIFWDNYIKCYRNNDKMFYCPND
WVGYNNVCYYFSNGSFFKNYTDANNYCKKINSTLA
NNDTNLLSLTKIYNNQSMYWVNNTVALRGDSVYAQ
RKVNNSDLLFICSK*
Warmbaths EP153R protein
SEQ ID No. 225
MYFKKKYIGLIDKNCEKKILDDCTTIKICYILIGI
LIGTNMITLIYNFIFWDHYMTCNKKDKMFYCPKDW
VGYNNVCYYFSNDSFTKNYTYASNSCKKINSTLAN
NDTNLLNLIKVYNNVSEYWVNNTVTLRGDHKYINK
QNNCNTEFLFICSR*
Tengani 62 EP153R protein
SEQ ID No. 226
MFSNKKYPSLIEKKMDDLMTLKFCYIIIAILIMTN
IFSLVVNIWGGGDRQSYENVFYCPKDWVGYNNACY
YFSNDMKNYTEASNYCKNLYNSTIVNNNTNIVNLT
KTLNLTKIYNHESNYWVNYSLINNESLTLRDSNFP
SSGKRTYIDLLYICSI*
Pretorisuskop/96/4 EP153R protein
SEQ ID No. 227
MYFKKKYIGLIDKNCEKKILDDCTTIKICYILIGI
LIGTNMITLIYNFIFWDHYMTCNKKDKMFYCPKDW
VGYNNVCYYFNNDSKNYTTATNSCKQLNSTLANND
TNLLNLTKVYHHDKLYWVNYSLNDNFSLSLRNSTY
EKRSKYLPLLFICSK*
Kenya 1950 EP153R protein
SEQ ID No. 228
MFLNKKYPSLIEKKMDDLMTLKFCYLIITFLIITN
IFSLAINIWGGGDMIDRQSCENIFYCPKDWVGYNN
ACYYFSNNNKNYTDANNYCKNSHNSTLANNDTKLL
NLTKLLNLSKLYYNDSTYWVKYSLPKNKAVTLRNS
TYKYDRVKYTETFFICSN*

Suitably, the EP153R gene encodes a protein comprising the sequence of SEQ ID No. 20, 217, 218, 219, 220, 221, 222, 223, 224, 225 226, 227 or 228. Suitably, the EP153R gene encodes a protein comprising a sequence having at least 70%, at least 80%, at least 90% or at least 95% identity with SEQ ID No. 20, 217, 218, 219, 220, 221, 222, 223, 224, 225 226, 227 or 228. Suitably, the EP153R gene encodes a protein consisting of the sequence of SEQ ID No. 20, 217, 218, 219, 220, 221, 222, 223, 224, 225 226, 227 or 228.

EP153R is immunogenic (i.e. evokes an immune response) (Burmakina et al. 2019 J. Gen. Virol. 100: 259-265). EP153R inhibits capsase-3 activation during ASFV infection and thereby has an inhibitory effect on apoptosis. EP153R is required for haemadsorption. EP153R also inhibits MHC-I expression in infected cells.

### EP402R

The EP402R gene encodes a protein which is incorporated in the external layer of the virus and is partly similar to the mammalian T-lymphocyte surface adhesion receptor CD2. In particular, the N-terminal extracellular region of the EP402R protein consists of two immunoglobulin-like (Ig-like) domains similar to the extracellular ligand-binding region of CD2. The N-terminal extracellular domain of the EP402R protein may be referred to as the “ligand-binding domain”. The cytoplasmic domain of EP402R protein is dissimilar to CD2. The EP402R protein may be referred to as CD2v due to this similarity.

EP402R is immunogenic (i.e. evokes an immune response) (Netherton et al. 2019 Front. Immunol. 10, 1318). EP402R is required for haemadsorption and may have a role in virus entry or spread. EP402R can bind the host protein AP-1. The functions of EP402R may be mediated by its extracellular, N-terminal, Ig-like domain binding to ligands in the same manner that mammalian CD2 binds extracellular adhesion molecules.

The gene (i.e. nucleotide) sequences and positions in the genome of EP402R genes from different ASFV strains are presented below.

Georgia 2007/1 EP402R
(NC_044959.1:73369-74451)
SEQ ID No. 229
ATGATAATACTTATTTTTTTAATATTTTCTAACAT
AGTTTTAAGTATTGATTATTGGGTTAGTTTTAATA
AAACAATAATTTTAGATAGTAATATTACTAATGAT
AATAATGATATAAATGGAGTATCATGGAATTTTTT
TAATAATTCTTTTAATACACTAGCTACATGTGGAA
AAGCAGGTAACTTTTGTGAATGTTCTAATTATAGT
ACATCAATATATAATATAACAAATAATTGTAGCTT
AACTATTTTTCCTCATAATGATGTATTTGATACAA
CATATCAAGTAGTATGGAATCAAATAATTAATTAT
ACAATAAAATTATTAACACCTGCTACTCCCCCAAA
TATCACATATAATTGTACTAATTTTTTAATAACAT
GTAAAAAAAATAATGGAACAAACACTAATATATAT
TTAAATATAAATGATACTTTTGTTAAATATACTAA
TGAAAGTATACTTGAATATAACTGGAATAATAGTA
ACATTAACAATTTTACAGCTACATGTATAATTAAT
AATACAATTAGTACATCTAATGAAACAACACTTAT
AAATTGTACTTATTTAACATTGTCATCTAACTATT
TTTATACTTTTTTTAAATTATATTATATTCCATTA
AGCATCATAATTGGGATAACAATAAGTATTCTTCT
TATATCCATCATAACTTTTTTATCTTTACGAAAAA
GAAAAAAACATGTTGAAGAAATAGAAAGTCCACCA
CCTGAATCTAATGAAGAAGAACAATGTCAGCATGA
TGACACCACTTCCATACATGAACCATCTCCCAGAG
AACCATTACTTCCTAAGCCTTACAGTCGTTATCAG
TATAATACACCTATTTACTACATGCGTCCCTCAAC
ACAACCACTCAACCCATTTCCCTTACCTAAACCGT
GTCCTCCACCCAAACCATGTCCGCCACCCAAACCA
TGTCCTCCACCTAAACCATGTCCTTCAGCTGAATC
CTATTCTCCACCCAAACCACTACCTAGTATCCCGC
TACTACCCAATATCCCGCCATTATCTACCCAAAAT
ATTTCGCTTATTCACGTAGATAGAATTATTTAA
China/2018/AnhuiXCGQ EP402R
(MK128995.1:73383-74465)
SEQ ID No. 230
ATGATAATACTTATTTTTTTAATATTTTCTAACAT
AGTTTTAAGTATTGATTATTGGGTTAGTTTTAATA
AAACAATAATTTTAGATAGTAATATTACTAATGAT
AATAATGATATAAATGGAGTATCATGGAATTTTTT
TAATAATTCTTTTAATACACTAGCTACATGTGGAA
AAGCAGGTAACTTTTGTGAATGTTCTAATTATAGT
ACATCAATATATAATATAACAAATAATTGTAGCTT
AACTATTTTTCCTCATAATGATGTATTTGATACAA
CATATCAAGTAGTATGGAATCAAATAATTAATTAT
ACAATAAAATTATTAACACCTGCTACTCCCCCAAA
TATCACATATAATTGTACTAATTTTTTAATAACAT
GTAAAAAAAATAATGGAACAAACACTAATATATAT
TTAAATATAAATGATACTTTTGTTAAATATACTAA
TGAAAGTATACTTGAATATAACTGGAATAATAGTA
ACATTAACAATTTTACAGCTACATGTATAATTAAT
AATACAATTAGTACATCTAATGAAACAACACTTAT
AAATTGTACTTATTTAACATTGTCATCTAACTATT
TTTATACTTTTTTTAAATTATATTATATTCCATTA
AGCATCATAATTGGGATAACAATAAGTATTCTTCT
TATATCCATCATAACTTTTTTATCTTTACGAAAAA
GAAAAAAACATGTTGAAGAAATAGAAAGTCCACCA
CCTGAATCTAATGAAGAAGAACAATGTCAGCATGA
TGACACCACTTCCATACATGAACCATCTCCCAGAG
AACCATTACTTCCTAAGCCTTACAGTCGTTATCAG
TATAATACACCTATTTACTACATGCGTCCCTCAAC
ACAACCACTCAACCCATTTCCCTTACCTAAACCGT
GTCCTCCACCCAAACCATGTCCGCCACCCAAACCA
TGTCCTCCACCTAAACCATGTCCTTCAGCTGAATC
CTATTCTCCACCCAAACCACTACCTAGTATCCCGC
TACTACCCAATATCCCGCCATTATCTACCCAAAAT
ATTTCGCTTATTCACGTAGATAGAATTATTTAA
Warthog EP402R (AY261365.1:73281-74450)
SEQ ID No. 231
ATGATAATAATCCTTATTTTTTTAATATATTTAAA
ACTGGTCTTAAATAATAATATTATTATATGGCATC
CTACTTTAAATGAAACAATTTTTTTAAATGTTACA
ACTATAGGGAATATTAGTGGTCTATTTTGGAATAC
ATATTATAATAATAATCGTAGTGAACTTACTAAGT
GCGGAATATATAATAATTATTGTTCATGTGATAGT
CTTAATACATCATTGTATAATATAAATAATTGTAG
TTTAGTTATTTTTCCAAATAATACAAAAATATTTA
ATACAATATATGAATTAATATATTCAGGTAATCAA
ACTAATTATACAATAAAATTGCTAGAATCTACTAC
TCCTCCAAATATTACATATAATTGTACTACATCTG
TAATAACATGTAAAAAAAATAATGGGACAAATACT
AATATATTTTTAACTATAAATAATACGATTGTTAA
TTCTACTAATGAAGATGTTATTTACTACTATTGGA
ATAGAAGTGAATTTAATAACTTTACAGCTACATGT
ATGATTAATAATACAATTAATTCATCAAATATATC
AGAAATTATAGACTGTACTAATACATTATTATTAA
ACAGTTATTTAGACTTTTTTCAAGTAGCCGGTTAT
ATGTTTTATATGATAATTTTTATTGTAACTGGAAT
AACAGTAAGTATTTTTATTTCAATCATAACTTTTT
TATCTTTACGAAAAAGAAAAAAACATGTTGAAGAA
ATAGAAAGTCTACCACCTGAATCTAATGAAGAAGA
ATGTCAGCATGATGATGACACCACTTCCATACATG
AACCATCCCCCAGAGAACCATTACTTCCTAAACCT
TACAGTCGTTATCAGTATAATACACCTATTTACTA
CATGCGTCCCTCAACACAACCACTCAACCCATTTC
CCCTACCTAAACCATGCCCACCACCTAAACCGTGT
CCGCCACCCAAACCATGTCCTCCACCCAAACCATG
CCCGCCACCCAAACCATGCCCGCCACCTAAACCAT
GTCCTCCACCTGAATCATGTTCTTCACCTGAATCA
TGTTCTTCACCTGAATCATATTCTCCACCCAAACC
ACTACCTAGTATCCCGCTGCTACCCAATATCCCGC
CATTATCTACACAAAATATTTCGCTTATTCATGTA
GATAGAATTATTTAA
Pretorisuskop/96/4 EP402R
(AY261363.1:74089-75309)
SEQ ID No. 232
ATGATAATGTTCATAACACTTATTTTTTTAAGTTA
TATTAATATAGTTTTAAGTAATAATTATTGGGCTA
GACTTAACGAAACAATAACTTTAAATAGTAACATT
ACTAATGATACTAATAATGAATTAGGTATATTTTG
GAATTCTTATAATAATACTTATTATAATAATACAT
TTAATAATATTGCTATATGTGGAAAAAAAGGTATT
TTTTGTGAATGTAATATTAATTATAATACATCTAT
ATCTAATACATCTATATCTAATACATCAATATATA
ATGTAACAAATAATTGTAGTTTAACGATTTTTCTT
TATGATGATAATATATTTAAAACATATCAATTAGT
ATATCAGAATTACAAAATTAATTATACAATAAACT
TATTATTACCTGTTACTTCTCCAAATATTACATAT
AATTGTACTAACTCTTTAATAACATGTGAAAAAAA
TGATGGGACAAACACTAATATGTTTTTATCTATAA
ATAATATAACCATTAATCATACTAATCAAGATATT
CTTACTTACTATTGGAATAATAGTGAGTTTAATAA
TTTTACAGCTACATGTATGATTAATAATACACTTA
ATTCAGCAAATACCACAAAAGTTATCAATTGCACT
AATCCATTATTAAATTCTTACCAAAATTATTTTCT
TGAAAATATTCATACATTATTTTATATCATAATTT
TTATTGTGAGTGGATTAATAGCAAGTATTTTTATT
TCAATTATAACTTTTTTATCTTTACGAAAAAGAAA
AAAACATGTTGAAGAAATAGAAAGTCCACCACCTG
AATCTAATGAAGAAGAACAATGTCAGCATGATGAC
ACCACTTCCATACATGAACCATCTCCCAGAGAACC
ATTACTTCCTAAACCTTACAGTCGTTATCAATATA
ATACACCTATTTACTACATGCGTCCCTCAACACAA
CCACTCAACCCATTTCCCCTACCTAACCCATGTCC
TCCACCTAAACCGTGTCCTCCACCCAAACCATGCC
CGCCACCTAAACCATGTCCCCCACCCAAACCATGC
CCGCCACCTAAACCGTGTCCTCCACCCAAACCATG
TCCACCACCTAAACCATGTTCTTCACCTGAATCAT
ATTCTCCACCCAAACCACTACCTAGTATCCCGCTA
CTACCCAATATCCCACCATTATCTACACAAAATAT
TTCACTTATTCATGTAGATAGAATTATTTAA
L60 EP402R (NC_044941.1:67916-69037)
SEQ ID No. 233
ATGATAATAATAGTTATTTTTTTAATGTGTTTAAA
AATAGTTTTAAACAATATTATAATATGGAGTACTT
TAAATCAAACTGTATTTTTAAATAATATTTTTACA
ATTAATGATACATATGGTGGTCTATTTTGGAATAC
ATATTATGATAATAATCGTAGTAATTTTACTTATT
GTGGAATAGCAGGAAATTATTGTTCATGTTGTGGT
CATAACATATCATTGTATAATACAACAAATAATTG
TAGTTTAATTATTTTTCCTAACAATACAGAAATAT
TTAATAGAACATATGAATTAGTATATTTGGACAAA
AAAATTAATTATACAGTAAAACTATTAAAATCTGT
TGATTCCCCAACTATTACATATAATTGTACTAATT
CTTTAATAACATGTAAAAATAATAATGGGACAAAT
GTTAATATATATTTAATTATTAATAATACAATTAG
TTCATTGAATGAAACAGAAAATATAAATTGTACTA
ATCCAATATTAAAATATCAAAATTATTTATCCACA
TTATTTTATATCATAATTTTTATTGTGAGTGGATT
AATAATAGGTATTTTTATTTCAATCATATCTGTAT
TATCTATACGAAGAAAAAGAAAAAAACATGTTGAA
GAAATAGAAAGTCCACCACCCTCTGAATCTAATGA
AGAAGATATTTCTCACGATGACACCACTTCCATAC
ATGAACCATCTCCCAGAGAACCATTACTTCCTAAG
CCTTACAGTCGTTATCAGTATAATACACCTATTTA
CTACATGCGTCCCTCAACACAACCACTCAACCCAT
TTCCCCTACCTAAACCATGCCCGCCACCTAAACCA
TGTCCTCCACCCAAGCCATGCCCGCCACCCAAACC
ATGTCCTCCACCTAAACCGTGTTCTCCACCCAAAC
CGTGTCGTCCACCTAAACCATGTCCTCCACCTAAA
CCATGTCCTCCACCTAAACCATGTCCTCCACCTAA
ACCATGTCCTCCATCCAAACCATGTCCTTCACCTG
AATCCTATTCTCCACCCAAACCACTACCTAGTATC
CCGTTACTACCCAATATCCCGCCATTATCTACACA
AAATATTTCGCTTATTCATGTAGATAGAATTATTT
AA
Benin 97/1 EP402R (NC_044956.1:67567-68775)
SEQ ID No. 234
ATGATAATAATAGTTATTTTTTTAATGTGTTTAAA
AATAGTTTTAAACAATATTATAATATGGAGTACTT
TAAATCAAACTGTATTTTTAAATAATATTTTTACA
ATTAATGATACATATGGTGGTCTATTTTGGAATAC
ATATTATGATAATAATCGTAGTAATTTTACTTATT
GTGGAATAGCAGGAAATTATTGTTCATGTTGTGGT
CATAACATATCATTGTATAATACAACAAATAATTG
TAGTTTAATTATTTTTCCTAACAATACAGAAATAT
TTAATAGAACATATGAATTAGTATATTTGGACAAA
AAAATTAATTATACAGTAAAACTATTAAAATCTGT
TGATTCCCCAACTATTACATATAATTGTACTAATT
CTTTAATAACATGTAAAAATAATAATGGGACAAAT
GTTAATATATATTTAATTATTAACAATACAATTGT
TAATGATACTAATGGAGATATCCTTAATTATTATT
GGAATGGTAATAATAATTTTACAGCTACATGTATG
ATTAATAATACAATTAGTTCATTGAATGAAACAGA
AAATATAAATTGTACTAATCCAATATTAAAATATC
AAAATTATTTATCCACATTATTTTATATCATAATT
TTTATTGTGAGTGGATTAATAATAGGTATTTTTAT
TTCAATCATATCTGTATTATCTATACGAAGAAAAA
GAAAAAAACATGTTGAAGAAATAGAAAGTCCACCA
CCCTCTGAATCTAATGAAGAAGATATTTCTCACGA
TGACACCACTTCCATACATGAACCATCTCCCAGAG
AACCATTACTTCCTAAGCCTTACAGTCGTTATCAG
TATAATACACCTATTTACTACATGCGTCCCTCAAC
ACAACCACTCAACCCATTTCCCCTACCTAAACCAT
GCCCGCCACCTAAACCATGTCCTCCACCCAAGCCA
TGCCCGCCACCCAAACCATGTCCTCCACCTAAACC
GTGTTCTCCACCCAAACCGTGTCGTCCACCTAAAC
CATGTCCTCCACCTAAACCATGTCCTCCACCTAAA
CCATGTCCTCCACCTAAACCATGTCCTCCATCCAA
ACCATGTCCTTCACCTGAATCCTATTCTCCACCCA
AACCACTACCTAGTATCCCGTTACTACCCAATATC
CCGCCATTATCTACACAAAATATTTCGCTTATTCA
TGTAGATAGAATTATTTAA
Tengani 62 EP402R (AY261364.1:68693-69841)
SEQ ID No. 235
ATGATAATAAAGGTTATTTTTTTAGTTTTCTTTAA
AATGGTTTTAAGTAATATTATAATATGGAGTACTT
TAAATAACACAATATATTTAAATATTAATTATTCA
GATAGTAATTTATATACTGGTCTATTTTGGAATAA
ATATTATAATAATACTCGTAATAATTTTACTACTT
GTGTAACATCAAATAGTAGCTATTGTACATGTAAA
GGCCATAACACATCATTGTATAATATAACAAATAA
TTGTAGTTTAATCATTTTTCCAAATAATACAAAAA
TATTTAATACAACATATGAATTAGTATATTTAAAT
AATAAAATTAATTATACAATACAAATGGTACAACC
TGTTGATCCTCCAATTATTATATATAATGATACTA
ATAATTCTTTAATAACATGTGAAAAAAATAATGGA
ACGAATACTGAAATCTATTTATATTTAAATGATAC
ATTTATTAATAATACTAATGAAAATAGTATTAAAT
ACTATTGGAATTGTAGCGAATTAAACCATAATATT
ACAACTACATGTATTATTAATAATACACTTAATTC
GGCAAATACCACAAAAGTTATAAATTGCACTAATC
TATTGTTAAAATCTGACCAAAATTATTTTCTTAAA
AATATTCATACATTATTTTATATCATAATTTTTAT
TGTGGGTGGAACATTAATAAGCATTATTATATCAA
TCATAACTTTTTTATCTTTACGAAAAAGAAAAAAA
CATGTTGAAGAAATAGAAAGTCCACCACCCTCTGA
ATCTAATGAAGAAGAACATTGTCAGCATGATGATA
CCACTTCCATACATGAACCATCTCCCAGAGAACCA
TTACTTCCTAAGCCTTACAGTCGTTATCAGTATAA
TACACCTATTTACTACATGCGTCCCTCAACACAAC
CACTCAACCCATTTCCCCTACCTAAACCATGTCCT
CCACCTAAACCGTGTCCTCCACCCAAACCATGTCC
TCCACCTAAACCGTGTCCTCCACCTAAACCGTGTC
CCCCACCTAAACCATGTCCTTCACCTGAATCCCAT
TCTCCACCCAAACCACTACCTAGTATCCCGCTGCT
ACCCAATATTCCCCCATTATCTACACAAAATATTT
CGCTTATTCATGTAGATAGAATTATTTAA
Malawi Lil-20/1 (1983)
EP402R (AY261361.1:72275-72458)
SEQ ID No. 236
ATGATAATAATACTTATTTTTTTAATAATTCCTAA
CATAGTTTTAAGTATTGATTATTGGGTTAGTTTTA
ATAAAACAATAATTTTAGATAGTAATATTACTAAT
GATAATAATGATATAAATGGAGTATCATGGAATTT
CCTTAATAATTCTCTTAATACACTAGCTACATGTG
GAAAAGCAGGTAACTTTTGTGAATGCTCTAATTAT
AGTACATCACTATATAATATAGCACATAATTGTAG
CTTAACTATTTTCCCTCATAATGATGTATTTGGTA
CACCATATCAAGTAGTATGGAATCAAATAATTAAT
TATACAATAAAATTATTAACACCTGTTACTCCCCC
AAATATTACATATAATTGTACTAATTTTTTAATAA
CATGTAAAAAAAATAATGGAACAAATACTATTATA
TATTTCAATATAAATGATACTAATGTTAAATATAC
TAATGAAAGTATACTTGAATATAACTGGAATAATA
GTAACTTTAACAATTTTACAGCTACATGTATAATT
AATAATACAATTAATTCATCAAATGATACACAAAC
TATAGACTGCATCAATACATTATTAAGCAGTTATT
TAGACTTTTTTCAAGTAGCCAGTTATATGTTTTAT
ATGATAATTTTTATTGCAACTGGAATAATAGCAAG
TATTTTTATATCAATCATAACTTTTTTATCTTTAC
GAAAAAGAAAAAAACATGTTGAAGAAATAGAAAGT
CCATCGCCATCTGAATCTAATGAAGAAGAACAATG
TCAGCATGATGACACCACTTCCATACATGAACCGT
CTCCCAGAGAACCATTACTTCCTAAGCCTTACAGT
CGATATCAGTATAATACACCTATTTACTATATGCG
TCCCTTAACACAACCACTCAATCCATCTCCCCTAC
CCAAACTGTGTCCTCCACCCAAACCGTGTCCTCCA
CCCAAACCGTGTCCTCCACCCAAACCGTGTCCTCC
ACCCAAACCGTGTCCTTCATCTGAATCATGTTCTC
CACCTGAATCATATTCTCTACCCAAACCACTACCT
AATATCCCGCTACTACCCAATATCCCGCCATTATC
TACACAAAATATCTCGCTTATTCACGTAGATAGAA
TTATTTAA
Ken06.Bus EP402R
(NC_044946.1:69827-70984)
SEQ ID No. 237
ATGATAATAAAGCTTATTTTTTTAATATCATTTAA
AATGATTTTAGGTATTGATTATTGGGTTAGTCTTA
ATAATACAATAATTTTAGATAGTAACATTACTATT
AATAATATTACTAATACTACTAATAATCCTACATT
AAATGGTATATTTTGGAATATTTATAATAATAGTT
ATAATAATACTTTTAATTTACTTACCACATGTGGG
AATACATATAATATATGTTCTTGTTCTAATAATTA
TAATACAATATTATTTAATTATAATACCACAAATA
ATTGTAGTTTAATTATTTCTCCTCATGATGAAAAA
ATTTTTGATACGATGTTTCAGATAATATATTTAAC
TAATAAAATTAATTATACAATACGATGGTTACAAC
CTGTTGATCCCCCAAATATTTCATTTAATGATAGT
AATTCTTTAATAAAATGTGAAAAAAATAATGGAAC
AAATACTGAAATCTATTTATATTTAAATGATACAT
TTATTAATAATACTAATGAAAATGATCTTAAATAT
TATTGGAATTGTAGTGAGTTAAACTATAATATTAC
AGCTACATGTATTATTAATAATACACTTAATTCGG
CAAATACCACAAAAGTTATAAATTGCACTAATCTA
TTATTAAAATCTGACCAAAATTATTTTCTTAAAAA
CATTCATACATTATTTTATATCATAATTTTTATTG
TAACTGGGATAATAATAAGTATTTTTATAGCAATC
ATAACTTTTTTATCTTTACGAAAAAGAAAAAAACA
TGTTGAAGAAATAGAAAGTCCATCGCCTGAATCTA
ATGAAGAGGAAGAACAACAACATCATCATGACACT
ACTTCCATACATGAACCGTCTCCCAGAGAACCATT
ACTTCCTAAGCCTTACAGTCGTTATCAGTATAATA
CACCTATTTATTACATGCGTCCCTCAACACAACAA
CTATTTAAATCATATTCTTTACCCAAACCATGTCC
CCCACCTAAACCATGTCCTCCACCTAAACCATGTC
CCCCACCCAAACCATGTCCCTCACCAGAATCATAT
CCCTCACCTGAACCATATCCTCTACTACCTAATAT
CCCACTACCACCCAATATCCCGCCATTATCTACAC
AAAATATTTCGCTTATTCACGTAGATAGAATTATT
TAA
Ken05/Tk1 EP402R (NC_044945.1:74107-75249)
SEQ ID No. 238
ATGATAATAAAACTTATTTTTTTAATTTGTTTTAA
AATAGTTTTAAGTATTGATAATAAAACAAAATTTA
ATGAAACATTAATTTTAGATAATATTGATTATTGG
GTAAAGTTTAATGATACAATAACTTTAGATAGTAA
TATTACTAGTGAAATAGAAGCTGTATCTTGGAATT
TTTATAATAATACTTTTAATCTACTTGCTACATGT
GGAAAAGCAAGTAACTTTTGTAGTTGTTCTAATTA
TAGTACATCATTTTTTAATATTACAAATAATTGTA
GTTTAACTATTTTTTTAATAGACGAGACGTTATTT
AATACAACATATCAAATAGTATATTCAACTAATAT
AATTAATTATAAAATAAACTTATTAATACCTGTTA
CTCCTCCAATTATTTCATATAATTGTACTAATTGT
TCTATAAATTGTAAAAAAAGTAATGGTACAAATAC
TAATATTTTTTTATCTATAAATGATACAATTGTTA
CATATACTAATGAAAGTATTCTTAATTATGATTAT
AACTGTAGTATTTTAAATAATAATTTTACAGTTAC
ATGTATAATTAATAATACAATTAGTACATCAAATA
CTACAGAATTTATAAACTGCACTAATATATTATTA
AGTAGTTATCTAGATTTTTTTCAAGTAACTAGTTA
TATATTTTATATGATAATTTTTATTGTAACTGGAA
TAACAGTAAGTATTCTTATATCCATCATAACTTTC
TTATTTATACGAAAAAGAAAACATGTTGAAGAAAT
AGAAAGTCCACCGCCTGAATCTAATGAAGAAGAAC
AACAATGTCATCATGACACCACTTCTATACATGAA
CCGTCTCCCAGAGAACCATTACTTCCTAAGCCTTA
CAGTCGTTATCAATATAATACACCTATTTACTACA
TGCGTCCCTCAACACAACAACTATTTAAATCATAT
TCTTTACCCAAACCATGTCCCCCACCTAAACCATG
TCCTCCACCCAAACCATGTCCCCCACCCAAACCAT
GTCCTCCACCCAAACCATGTCCTCCATCCAAACCA
TATCCTTCACTACCTAGTATCCCGCTACCACCCGA
TATCCCGCCATTATCTACACAAAATATTTCGCTTA
TTCACGTAGATAGAATTATTTAA
Kenya 1950 EP402R (AY261360.1:76800-77978)
SEQ ID No. 239
ATGATAATAAAACTTATTTTTTTAATTTGTTTTAA
AATAGTTTTAAGTATTGATAATAAAACAAAATTTA
ATGAAACATTAATTTTAGATAATATTGATTATTGG
GTAAAGTTTAATGATACAATAACTTTAGATAGTAA
TATTACTAGTGAAATAGAAGCTGTATCTTGGAATT
TTTATAATAATACTTTTAATCTACTTGCTATATGT
GGAAAAGCAAGTAACTTTTGTAGTTGTTCTAATTA
TAGTACATCATTTTTTAATATTACAAATAATTGTA
GTTTAACTATTTTTTTAATAGACGAGACGTTATTT
AATACAACATATCAAATAGTATATTCAACTAATAT
AATTAATTATAAAATAAACTTATTAATACCTGTTA
CTCCTCCAATTATTTCATATAATTGTGCTAATTGT
TCTATAAATTGTAAAAAAAGTAATGGTACAAATAC
TAATATTTTTTTATCTATAAATGATACAATTGTTA
CATATACTAATGAAAGTATTCTTAATTATGATTAT
AACTGTAGTATTTTAAATAATAATTTTACAGTTAC
ATGTATAATTAATAATACAATTAGCACATCAAATA
CTACAGAATTTATAAACTGCACTAATATATTATTA
AGTAGTTATCTAGATTTTTTTCAAGTAACTAGTTA
TATATTTTATATGATAATTTTTATTGTAACTGGAA
TAACAGTAAGTATTCTTATATCCATCATAACTTTC
TTATTTATACGAAAAAGAAAACATGTTGAAGAAAT
AGAAAGTCCACCGCCTGAATCTAATGAAGAAGAAC
AACAATGTCATCATGACACCACTTCTATACATGAA
CCGTCTCCCAGAGAACCATTACTTCCTAAGCCTTA
CAGTCGTTATCAATATAATACACCTATTTACTACA
TGCGTCCCTCAACACAACAACTATTTAAATCATAT
TCTTTACCCAAACCATGTCCCCCACCTAAACCATG
TCCTCCACCTAAACCATGTCCCCCACCTAAACCAT
GTCCTCCATCCAAACCATGTCCTCCACCTGAACCA
TATTCTCCACCCAAACCATGTCCTCCACCCAAACC
ATATCCTTCACTACCTAGTATCCCGCTACCACCCG
ATATCCCGCCATTATCTACACAAAATATTTCGCTT
ATTCACGTAGATAGAATTATTTAA
Warthog
EP402R (AY261366.1 70011-71156)
SEQ ID No. 240
ATGATAATTAAACTTATTTTTTTAATTTGTTTCAA
AATAGTTTTAAGTATTAATTATTGGGTTAGGTATA
ATGATACAGTAACTTTAAATAGTAATATCAATAGC
GAGACAGAAGGTATATTTTGGAATTTTTATAATAA
TACTTTTAATACTATTGCCACATGTGGAAAAAAAA
ATAATGTTTGTGAATGTTCTAATTATGATAAATCC
TTATATAATATAACAAATAATTGTAGTTTAACAAT
TTTTCCAAATAATACAAAAATATTTAATACAACAT
ATCAATTAGTATATTCACGCAATAGAATTAATTAC
ACAATAAATTTGTTATTACCTGTTACATCTCCAAT
TATTACATATAATTGTACTCAATCTTTAATAACAT
GTGAAAAAACTAATGGAACAAATATTCATTTATTC
TTAAATTTAAATGATACAATTAATGAATATACTAA
CAAAAGTTTTCTCAATTACTATTGGAATAGTAGTG
AGTTAAATAATATTTTTCTTGCTACATGTATTATT
AATAATACACTTAATTCAGCAAATACTACAAAAGT
TATCAATTGCACTAACCCATTATTAAAATCTTACC
AAAATTATTTTCTTGAAAATATTCATACATTATTT
TATATGATAATTTTTATTGTATCTGGAATAACAAT
AAGTATTTTTATATCAATCATAACTTTTTTATCTT
TACGAAAAAGAAAAAAACATGTTGAAGAAATAGAG
AGTCCACCACCTGAATCTAATGAAGAAGAACAATG
TCAGCATGATGACACCACTTCTATACATGAACCAT
CTCCCAGAGAACCATTACTTCCTAAACCTTACAGT
CGTTATCAGTATAATACACCTATTTACTACATGCG
TCCCTCAACACAACCACTCAACCCATTTCCCCTAC
CTAAACCATGCCCGCCACCTAAACCGTGTCCTCCA
CCCAAACCATGCCCGCCACCCAAACCATGCCCGCC
ACCTAAACCATGTCCTCCACCCAAACCATGCCCGC
CACCTAAACCATGTCCTCCACCTGAATCATATTCT
CCACCCAAACCACTACCTAGTATCCCGCTGCTACC
CAATATCCCGCCATTATCTACACAAAATATTTCGC
TTATTCATGTAGATAGAATTATTTAA
Mkuzi 1979 EP402R (AY261362.1:75106-76287)
SEQ ID No. 241
ATGATGATAATTATACTTATTTTTTTAATATGTTT
AAAAATAGTTTTAAATAATAATATTATAATATGGC
ATACTTTAAATGAAACAATTTTTTTAAATAATAGT
ATTACATTTACGGATAATCTTGGTGGTCTATTTTG
GAATACATATTATAATAATAATCATAGTATCCTTA
CTAGTTGCGGAATATATCAAAATTATTGTTCATGC
ACTAATAATTATAGCACATCATTATATAATATAAC
AAATAATTGTGGTTTAATAATTTTTCCAAATAATA
CAAAAAGATTTAACACAACGTATGAATTAGTATAT
TTAAATAATAGTACTAAATATACAATAAACTTATT
AGTACCTGTTACTCCTCCAAATATTACATATAATT
GTACTACATCTGTAATAACATGTAAAAAAAATGAT
GGCACAAATACTAATATGTTTTTAAAAATAAATGA
TACAATCGTTAAAAGTAGTAATGAAAGTGTTTTTG
AATACTCTTGGAATAGTACTGATTTAAATAACAAT
TTTACGGCTATATGTATAATTAATAATACAATTAA
TTCATCAAATGACACACAAACTATAGAATGTCCCA
ATATATTATTAAACAATTATTTAGACTTTTTTCAA
GTAACTAGTTACATGTTTTATATGATAATTTTTAT
TGTAACTGGAATAATAGCAAGTATTTTTATTTCAA
TCATAACTTTTTTATCTTTACGAAAAAGAAAAAAA
CATGTTGAAGAAATAGAAAGTCCACCACCCTCTGA
ATCTAATGAAGAAGATATTTCTCACGATGACACCA
CTTCCATACATGAACCATCTCCCAGAGAACCATTA
CTTCCTAAGCCTTACAGTCGTTATCAGTATAATAC
ACCTATTTACTACATGCGTCCCTCAACACAACCAC
TCAACCCATTTCCCCTACCTAAACCATGCCCGCCA
CCTAAACCATGCCCGCCACCCAAACCATGTCCTCC
ACCTAAACCGTGTCCTCCACCCAAACCGTGTCGTC
CACCTAAACCATGTCCTCCACCTAAACCATGCCCA
CCACCTAAACCATGTCCTTCACCTGAAACATATTC
TCCACCCAAACCACTACCTAATATCCCGCTGCTAC
CCAATATCCCGCCATTATCTACACAAAATATTTCG
CTTATTCACGTAGATAGGATTATTTAA

In an embodiment the invention provides an attenuated ASF virus in which the expression and/or activity of the EP402R gene is disrupted. Suitably, the EP402R gene comprises the sequence of SEQ ID No. 229, 230, 231, 232, 233, 234, 235, 236, 237, 238, 239, 240 or 241. Suitably, the EP402R gene comprises a sequence having at least 70%, at least 80%, at least 90% or at least 95% identity with SEQ ID No. 229, 230, 231, 232, 233, 234, 235, 236, 237, 238, 239, 240 or 241. Suitably, the EP402R gene consists of the sequence of SEQ ID No. 229, 230, 231, 232, 233, 234, 235, 236, 237, 238, 239, 240 or 241.

The amino acid sequence of the EP402R protein from the Benin 97/1 strain is depicted below as SEQ ID No. 21.

Benin 97/1 EP402R protein
SEQ ID No. 21
MIIIVIFLMCLKIVLNNIIIWSTLNQTVFLNNIFT
INDTYGGLFWNTYYDNNRSNFTYCGIAGNYCSCCG
HNISLYNTTNNCSLIIFPNNTEIFNRTYELVYLDK
KINYTVKLLKSVDSPTITYNCTNSLITCKNNNGTN
VNIYLIINNTIVNDTNGDILNYYWNGNNNFTATCM
INNTISSLNETENINCTNPILKYQNYLSTLFYIII
FIVSGLIIGIFISIISVLSIRRKRKKHVEEIESPP
PSESNEEDISHDDTTSIHEPSPREPLLPKPYSRYQ
YNTPIYYMRPSTQPLNPFPLPKPCPPPKPCPPPKP
CPPPKPCPPPKPCSPPKPCRPPKPCPPPKPCPPPK
PCPPPKPCPPSKPCPSPESYSPPKPLPSIPLLPNI
PPLSTQNISLIHVDRII

Suitably, the EP402R gene encodes a protein comprising the sequence of SEQ ID No. 21. Suitably, the EP402R gene encodes a protein comprising a sequence having at least 70%, at least 80%, at least 90% or at least 95% identity with SEQ ID No. 21. Suitably, the EP402R gene encodes a protein consisting of the sequence of SEQ ID No. 21.

The ligand-binding domain of Benin 97/1 EP402R is formed by amino acids 1-198 of SEQ ID No. 21 which are presented below as SEQ ID No. 34.

Benin 97/1 EP402R protein
ligand-binding domain
SEQ ID No. 34
MIIIVIFLMCLKIVLNNIIIWSTLNQTVFLNNIFT
INDTYGGLFWNTYYDNNRSNFTYCGIAGNYCSCCG
HNISLYNTTNNCSLIIFPNNTEIFNRTYELVYLDK
KINYTVKLLKSVDSPTITYNCTNSLITCKNNNGTN
VNIYLIINNTIVNDTNGDILNYYWNGNNNFTATCM
INNTISSLNETENINCTNPILKY

Suitably, the ligand-binding domain of the EP402R protein comprises the sequence of SEQ ID No. 34. Suitably, the ligand-binding domain of the EP402R protein comprises a sequence having at least 70%, at least 80%, at least 90% or at least 95% identity with SEQ ID No. 34. Suitably, the ligand-binding domain of the EP402R protein consists of the sequence of SEQ 5 ID No. 34.

The orthologous EP402R genes from other ASFV isolates are depicted below as SEQ ID Nos 22 to 30:

Mkuzi EP402R protein
SEQ ID No. 22
MIIILIFLICLKIVLNNNIIIWHTLNETIFLNNSI
TFTDNLGGLFWNTYYNNNHSILTSCGIYQNYCSCT
NNYSTSLYNITNNCGLIIFPNNTKRFNTTYELVYL
NNSTKYTINLLVPVTPPNITYNCTTSVITCKKNDG
TNTNMFLKINDTIVKSSNESVFEYSWNSTDLNNNF
TAICIINNTINSSNDTQTIECPNILLNNYLDFFQV
TSYMFYMIIFIVTGIIASIFISIITFLSLRKRKKH
VEEIESPPPSESNEEDISHDDTTSIHEPSPREPLL
PKPYSRYQYNTPIYYMRPSTQPLNPFPLPKPCPPP
KPCPPPKPCPPPKPCPPPKPCRPPKPCPPPKPCPP
PKPCPSPETYSPPKPLPNIPLLPNIPPLSTQNISL
IHVDRII
Warmbaths EP402R protein
SEQ ID No. 23
MIIILIFLIYLKLVLNNNIIIWHPTLNETIFLNVT
TIGNISGLFWNTYYNNNRSELTKCGIYNNYCSCDS
LNTSLYNINNCSLVIFPNNTKIFNTIYELIYSGNQ
TNYTIKLLESTTPPNITYNCTTSVITCKKNNGTNT
NIFLTINNTIVNSTNEDVIYYYWNRSEFNNFTATC
MINNTINSSNISEIIDCTNTLLLNSYLDFFQVAGY
MFYMIIFIVTGITVSIFISIITFLSLRKRKKHVEE
IESLPPESNEEECQHDDDTTSIHEPSPREPLLPKP
YSRYQYNTPIYYMRPSTQPLNPFPLPKPCPPPKPC
PPPKPCPPPKPCPPPKPCPPPKPCPPPESCSSPES
CSSPESYSPPKPLPSIPLLPNIPPLSTQNISLIHV
DRII
Georgia 2007/1 EP402R protein
SEQ ID No. 24
MIILIFLIFSNIVLSIDYWVSFNKTIILDSNITND
NNDINGVSWNFFNNSFNTLATCGKAGNFCECSNYS
TSIYNITNNCSLTIFPHNDVFDTTYQVVWNQIINY
TIKLLTPATPPNITYNCTNFLITCKKNNGTNTNIY
LNINDTFVKYTNESILEYNWNNSNINNFTATCIIN
NTISTSNETTLINCTYLTLSSNYFYTFFKLYYIPL
SIIIGITISILLISIITFLSLRKRKKHVEEIESPP
PESNEEEQCQHDDTTSIHEPSPREPLLPKPYSRYQ
YNTPIYYMRPSTQPLNPFPLPKPCPPPKPCPPPKP
CPPPKPCPSAESYSPPKPLPSIPLLPNIPPLSTQN
ISLIHVDRII
Malawi Lil-20/1 (1983) EP402R protein
SEQ ID No. 25
MIIILIFLIIPNIVLSIDYWVSFNKTIILDSNITN
DNNDINGVSWNFLNNSLNTLATCGKAGNFCECSNY
STSLYNIAHNCSLTIFPHNDVFGTPYQVVWNQIIN
YTIKLLTPVTPPNITYNCTNFLITCKKNNGTNTII
YFNINDTNVKYTNESILEYNWNNSNFNNFTATCII
NNTINSSNDTQTIDCINTLLSSYLDFFQVASYMFY
MIIFIATGIIASIFISIITFLSLRKRKKHVEEIES
PSPSESNEEEQCQHDDTTSIHEPSPREPLLPKPYS
RYQYNTPIYYMRPLTQPLNPSPLPKLCPPPKPCPP
PKPCPPPKPCPPPKPCPSSESCSPPESYSLPKPLP
NIPLLPNIPPLSTQNISLIHVDRII
Kenya 1950 EP402R protein
SEQ ID No. 26
MIIKLIFLICFKIVLSIDNKTKFNETLILDNIDYW
VKFNDTITLDSNITSEIEAVSWNFYNNTFNLLAIC
GKASNFCSCSNYSTSFFNITNNCSLTIFLIDETLF
NTTYQIVYSTNIINYKINLLIPVTPPIISYNCANC
SINCKKSNGTNTNIFLSINDTIVTYTNESILNYDY
NCSILNNNFTVTCIINNTISTSNTTEFINCTNILL
SSYLDFFQVTSYIFYMIIFIVTGITVSILISIITF
LFIRKRKHVEEIESPPPESNEEEQQCHHDTTSIHE
PSPREPLLPKPYSRYQYNTPIYYMRPSTQQLFKSY
SLPKPCPPPKPCPPPKPCPPPKPCPPSKPCPPPEP
YSPPKPCPPPKPYPSLPSIPLPPDIPPLSTQNISL
IHVDRII
N10 EP402R protein
SEQ ID No. 27
MIIKLIFLISFKMILGIDYWVSLNNTIILDSNITI
NNITNTTNNPTLNGIFWNIYNNSYNNTFNLLTTCG
NTYNICSCSNNYNTILFNYNTTNNCSLIISPHDEK
IFDTMFQIIYLTNKINYTIRWLQPVDPPNISFNDS
NSLIKCEKNNGTNTEIYLYLNDTFINNTNENDLKY
YWNCSELNYNITATCIINNTLNSANTTKVINCTNL
LLKSDQNYFLKNIHTLFYIIIFIVTGIIISIFIAI
ITFLSLRKRKKHVEEIESPSPESNEEEEQQHHHDT
TSIHEPSPREPLLPKPYSRYQYNTPIYYMRPSTQQ
LFKSYSLPKPCPPPKPCPPPKPCPPPKPCPSPESY
PSPEPYPLLPNIPLPPNIPPLSTQNISLIHVDRII
Tengani 62 EP402R protein
SEQ ID No. 28
MIIKVIFLVFFKMVLSNIIIWSTLNNTIYLNINYS
DSNLYTGLFWNKYYNNTRNNFTTCVTSNSSYCTCK
GHNTSLYNITNNCSLIIFPNNTKIFNTTYELVYLN
NKINYTIQMVQPVDPPIIIYNDTNNSLITCEKNNG
TNTEIYLYLNDTFINNTNENSIKYYWNCSELNHNI
TTTCIINNTLNSANTTKVINCTNLLLKSDQNYFLK
NIHTLFYIIIFIVGGTLISIIISIITFLSLRKRKK
HVEEIESPPPSESNEEEHCQHDDTTSIHEPSPREP
LLPKPYSRYQYNTPIYYMRPSTQPLNPFPLPKPCP
PPKPCPPPKPCPPPKPCPPPKPCPPPKPCPSPESH
SPPKPLPSIPLLPNIPPLSTQNISLIHVDRII
Pr4 EP402R protein
SEQ ID No. 29
MFITLIFLSYINIVLSNNYWARLNETITLNSNITN
DTNNELGIFWNSYNNTYYNNTFNNIAICGKKGIFC
ECNINYNTSISNTSISNTSIYNVTNNCSLTIFLYD
DNIFKTYQLVYQNYKINYTINLLLPVTSPNITYNC
TNSLITCEKNDGTNTNMFLSINNITINHTNQDILT
YYWNNSEFNNFTATCMINNTLNSANTTKVINCTNP
LLNSYQNYFLENIHTLFYIIIFIVSGLIASIFISI
ITFLSLRKRKKHVEEIESPPPESNEEEQCQHDDTT
SIHEPSPREPLLPKPYSRYQYNTPIYYMRPSTQPL
NPFPLPNPCPPPKPCPPPKPCPPPKPCPPPKPCPP
PKPCPPPKPCPPPKPCSSPESYSPPKPLPSIPLLP
NIPPLSTQNISLIHVDRII
Warthog EP402R protein
SEQ ID No. 30
MIIKLIFLICFKIVLSINYWVRYNDTVTLNSNINS
ETEGIFWNFYNNTFNTIATCGKKNNVCECSNYDKS
LYNITNNCSLTIFPNNTKIFNTTYQLVYSRNRINY
TINLLLPVTSPIITYNCTQSLITCEKTNGTNIHLF
LNLNDTINEYTNKSFLNYYWNSSELNNIFLATCII
NNTLNSANTTKVINCTNPLLKSYQNYFLENIHTLF
YMIIFIVSGITISIFISIITFLSLRKRKKHVEEIE
SPPPESNEEEQCQHDDTTSIHEPSPREPLLPKPYS
RYQYNTPIYYMRPSTQPLNPFPLPKPCPPPKPCPP
PKPCPPPKPCPPPKPCPPPKPCPPPKPCPPPESYS
PPKPLPSIPLLPNIPPLSTQNISLIHVDRII
China/2018/AnhuiXCGQ EP402R protein
SEQ ID No. 242
MIILIFLIFSNIVLSIDYWVSFNKTIILDSNITND
NNDINGVSWNFFNNSFNTLATCGKAGNFCECSNYS
TSIYNITNNCSLTIFPHNDVFDTTYQVVWNQIINY
TIKLLTPATPPNITYNCTNFLITCKKNNGTNTNIY
LNINDTFVKYTNESILEYNWNNSNINNFTATCIIN
NTISTSNETTLINCTYLTLSSNYFYTFFKLYYIPL
SIIIGITISILLISIITFLSLRKRKKHVEEIESPP
PESNEEEQCQHDDTTSIHEPSPREPLLPKPYSRYQ
YNTPIYYMRPSTQPLNPFPLPKPCPPPKPCPPPKP
CPPPKPCPSAESYSPPKPLPSIPLLPNIPPLSTQN
ISLIHVDRII
Pretorisuskop/96/4 EP402R protein
SEQ ID No. 243
MIMFITLIFLSYINIVLSNNYWARLNETITLNSNI
TNDTNNELGIFWNSYNNTYYNNTFNNIAICGKKGI
FCECNINYNTSISNTSISNTSIYNVTNNCSLTIFL
YDDNIFKTYQLVYQNYKINYTINLLLPVTSPNITY
NCTNSLITCEKNDGTNTNMFLSINNITINHTNQDI
LTYYWNNSEFNNFTATCMINNTLNSANTTKVINCT
NPLLNSYQNYFLENIHTLFYIIIFIVSGLIASIFI
SIITFLSLRKRKKHVEEIESPPPESNEEEQCQHDD
TTSIHEPSPREPLLPKPYSRYQYNTPIYYMRPSTQ
PLNPFPLPNPCPPPKPCPPPKPCPPPKPCPPPKPC
PPPKPCPPPKPCPPPKPCSSPESYSPPKPLPSIPL
LPNIPPLSTQNISLIHVDRII
L60 EP402R protein
SEQ ID No. 244
MIIIVIFLMCLKIVLNNIIIWSTLNQTVFLNNIFT
INDTYGGLFWNTYYDNNRSNFTYCGIAGNYCSCCG
HNISLYNTTNNCSLIIFPNNTEIFNRTYELVYLDK
KINYTVKLLKSVDSPTITYNCTNSLITCKNNNGTN
VNIYLIINNTISSLNETENINCTNPILKYQNYLST
LFYIIIFIVSGLIIGIFISIISVLSIRRKRKKHVE
EIESPPPSESNEEDISHDDTTSIHEPSPREPLLPK
PYSRYQYNTPIYYMRPSTQPLNPFPLPKPCPPPKP
CPPPKPCPPPKPCPPPKPCSPPKPCRPPKPCPPPK
PCPPPKPCPPPKPCPPSKPCPSPESYSPPKPLPSI
PLLPNIPPLSTQNISLIHVDRII
Ken06.Bus EP402R protein
SEQ ID No. 245
MIIKLIFLISFKMILGIDYWVSLNNTIILDSNITI
NNITNTTNNPTLNGIFWNIYNNSYNNTFNLLTTCG
NTYNICSCSNNYNTILFNYNTTNNCSLIISPHDEK
IFDTMFQIIYLTNKINYTIRWLQPVDPPNISFNDS
NSLIKCEKNNGTNTEIYLYLNDTFINNTNENDLKY
YWNCSELNYNITATCIINNTLNSANTTKVINCTNL
LLKSDQNYFLKNIHTLFYIIIFIVTGIIISIFIAI
ITFLSLRKRKKHVEEIESPSPESNEEEEQQHHHDT
TSIHEPSPREPLLPKPYSRYQYNTPIYYMRPSTQQ
LFKSYSLPKPCPPPKPCPPPKPCPPPKPCPSPESY
PSPEPYPLLPNIPLPPNIPPLSTQNISLIHVDRII
Ken05/Tk1 EP402R protein
SEQ ID No. 246
MIIKLIFLICFKIVLSIDNKTKFNETLILDNIDYW
VKFNDTITLDSNITSEIEAVSWNFYNNTFNLLATC
GKASNFCSCSNYSTSFFNITNNCSLTIFLIDETLF
NTTYQIVYSTNIINYKINLLIPVTPPIISYNCTNC
SINCKKSNGTNTNIFLSINDTIVTYTNESILNYDY
NCSILNNNFTVTCIINNTISTSNTTEFINCTNILL
SSYLDFFQVTSYIFYMIIFIVTGITVSILISIITF
LFIRKRKHVEEIESPPPESNEEEQQCHHDTTSIHE
PSPREPLLPKPYSRYQYNTPIYYMRPSTQQLFKSY
SLPKPCPPPKPCPPPKPCPPPKPCPPPKPCPPSKP
YPSLPSIPLPPDIPPLSTQNISLIHVDRII

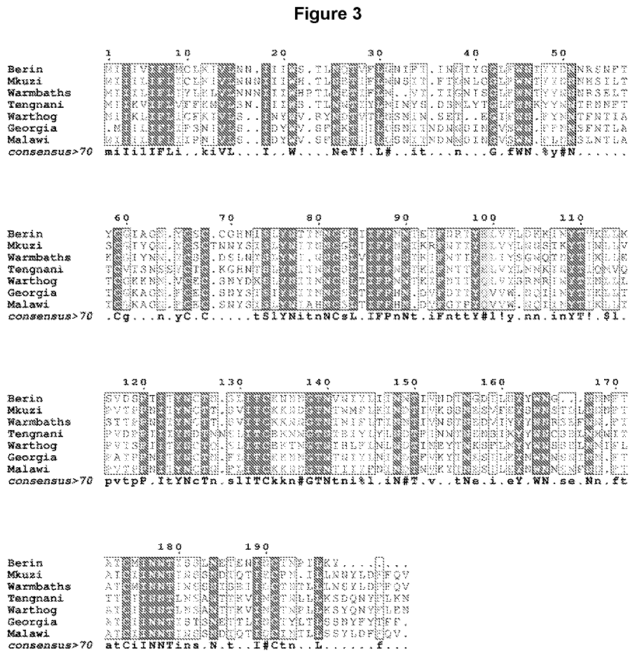

The ligand-binding domain of the EP402R protein from other strains can be readily identified by alignment with the sequence of Benin 97/1 EP402R protein, such as shown in FIG. 3.

In an embodiment the invention provides an attenuated ASF virus in which the expression and/or activity of the EP402R gene is disrupted. Suitably, the EP402R gene encodes a protein comprising the sequence of SEQ ID No. 22. Suitably, the EP402R gene encodes a protein comprising the sequence of SEQ ID No. 23. Suitably, the EP402R gene encodes a protein comprising the sequence of SEQ ID No. 24. Suitably, the EP402R gene encodes a protein comprising the sequence of SEQ ID No. 25. Suitably, the EP402R gene encodes a protein comprising the sequence of SEQ ID No. 26. Suitably, the EP402R gene encodes a protein comprising the sequence of SEQ ID No. 27. Suitably, the EP402R gene encodes a protein comprising the sequence of SEQ ID No. 28. Suitably, the EP402R gene encodes a protein comprising the sequence of SEQ ID No. 29. Suitably, the EP402R gene encodes a protein comprising the sequence of SEQ ID No. 30. Suitably, the EP402R gene encodes a protein comprising the sequence of SEQ ID No. 242. Suitably, the EP402R gene encodes a protein comprising the sequence of SEQ ID No. 243. Suitably, the EP402R gene encodes a protein comprising the sequence of SEQ ID No. 244. Suitably, the EP402R gene encodes a protein comprising the sequence of SEQ ID No. 245. Suitably, the EP402R gene encodes a protein comprising the sequence of SEQ ID No. 246.

### Haemadsorption

Haemadsorption is the phenomenon whereby cells infected with ASFV adsorb erythrocytes (red blood cells) on their surface. The degree of haemadsorption induced by an ASFV may be measured using a haemadsorption assay such as described herein. For example, cells may be transfected with a protein or infected with an ASFV, then red blood cells added and the degree of haemadsorption detected by imaging. In this way, different proteins and viruses can be tested for their effect on haemadsorption.

EP402R and EP153R are involved in mediating haemadsorption of ASFV-infected cells.

In an embodiment the invention provides an attenuated ASF virus which has reduced haemadsorption.

In an embodiment of the attenuated ASF virus of the invention, the ability of the EP153R gene and/or the EP402R gene to mediate haemadsorption may be disrupted. In an embodiment, the ability of the EP153R gene to mediate haemadsorption may be disrupted.

In an embodiment, the ability of the EP402R gene to mediate haemadsorption may be disrupted.

Reducing haemadsorption or disrupting the ability to mediate haemadsorption means that cells infected with the attenuated ASFV of the invention or transfected with a mutant, non-functional EP153R or EP402R protein adsorb fewer red blood cells to their surface than cells infected with a wild-type ASFV or transfected with a wild-type EP153R or EP402R protein. The number of red blood cells adsorbed to the surface of the infected/transfected cells may be decreased by at least 50, 60, 70, 80 or 90%. In an embodiment, haemadsorption is abolished i.e. no red blood cells adsorb to the surface of cells infected with the attenuated ASFV of the invention or transfected with a mutant, non-functional EP153R or EP402R protein.

### Gene Expression

The attenuated African Swine Fever (ASF) virus of one embodiment of the present invention has disrupted expression and/or activity of the genes DP148R, EP153R and EP402R. These genes may be referred to herein as the “disrupted genes”.

In an embodiment the invention provides an attenuated ASFV in which expression of the genes DP148R, EP153R and EP402R is disrupted.

The term “expression” with respect to a gene refers to the ability of the ASF virus to produce the product of the gene, such as RNA and/or protein. Disruption of expression of a gene means that production of the gene product is decreased. Expression of the gene may be decreased by at least 50%, at least 60%, at least 70%, at least 80%, at least 90% and/or at least 95%. Expression of the gene may be decreased to the extent that production of the gene product, such as RNA and/or protein, is entirely abolished (i.e. the gene product is not produced at all). Disruption of gene expression decreases expression of the gene relative to the expression of the gene when it is not disrupted. For example, a mutated gene may have decreased expression in comparison to a wild-type version of the gene.

A gene the expression of which is disrupted may not be fully transcribed and translated. Transcription of the gene may be decreased by at least 50%, at least 60%, at least 70%, at least 80%, at least 90% and/or at least 95%. Transcription of the gene may be abolished (i.e. the gene may not be transcribed). Translation of the gene may be decreased by at least 50%, at least 60%, at least 70%, at least 80%, at least 90% and/or at least 95%. Translation of the gene may be abolished. The gene may be transcribed but not translated. The gene may be transcribed and translated but the protein too rapidly degraded to carry out its function. The gene may be transcribed and translated but the protein may be non-functional.

Gene expression may be measured by techniques known in the art. For example, the amount of mRNA transcribed from a gene may be quantified, such as by using quantitative polymerase chain reaction (qPCR). Alternatively or additionally, the amount of protein may be quantified, such as by using Western blotting or mass spectrometry.

### Gene Activity

In an embodiment the invention provides an attenuated ASFV in which activity of the genes DP148R, EP153R and EP402R is disrupted.

The term “activity” with respect to a gene refers to the ability of the gene to carry out its functions. Different genes have different activities i.e. different functions they fulfil. A given gene may have multiple activities; disruption of gene activity means disruption of one or more of those activities. One or more activity of the gene may be disrupted whilst one or more other activities are not disrupted. Disruption of gene activity decreases the activity of the gene relative to the activity of the gene when it is not disrupted. For example, a mutated gene may have decreased activity in comparison to a wild-type version of the gene. Gene activity may be decreased to the extent that gene activity is entirely abolished.

The attenuated ASFV according to the present invention may comprise a non-functional version of the disrupted genes.

Disruption of expression of a gene may also disrupt activity of that gene as the decreased amount of gene product means the gene cannot as effectively carry out one or more of its activities.

Gene expression and/or activity are disrupted by mutating the ASFV genome i.e. by mutating the ASFV genome. A “mutation” means a change in the nucleotide sequence of the ASFV genome relative to a known ASFV genotype. Mutations include changing one or more nucleotides to different nucleotides (i.e. substitution), adding nucleotides, deleting nucleotides and/or a combination of these.

In an embodiment the attenuated ASF virus of the invention comprises mutations that disrupt the expression and/or activity of the genes DP148R, EP153R and EP402R.

Gene expression and/or activity may be disrupted by disrupting transcription of the gene into mRNA i.e. by decreasing gene transcription, such as completely abolishing gene transcription. Gene expression and/or activity may be disrupted by disrupting translation of mRNA into protein. In an embodiment the attenuated ASF virus comprises mutations that decrease transcription and/or translation of the genes. In an embodiment the attenuated ASF virus comprises mutations that cause the genes to not be transcribed and/or translated (i.e. complete abolition of transcription and/or translation).

Gene expression and/or activity may be disrupted by mutating a non-coding sequence associated with the gene, such as a promoter. In an embodiment the attenuated ASF virus comprises mutations in promoters of one or more of the disrupted genes.

Gene expression and/or activity may be disrupted by mutating a coding sequence of one or more of the disrupted genes.

### Activity of DP148R

In an embodiment of the attenuated ASFV of the invention, DP148R activity may be disrupted.

In an embodiment the activity of DP148R that may be disrupted is its ability to inhibit type I interferon. In other words, the ability of DP148R to inhibit type I interferon may be decreased. The ability of DP148R to inhibit type I interferon may be decreased by at least 50, 60, 70, 80 or 90%. The ability of DP148R to inhibit type I interferon may be completely abolished.

In an embodiment the activity of DP148R that may be disrupted is its ability to inhibit NF-κB activity. In an embodiment, the invention provides an attenuated ASF virus in which the ability of the DP148R gene to inhibit NFκB activity is disrupted. NFκB activity may be measured using a luciferase reporter assay as described herein.

Disruption of the ability to inhibit NFκB activity means that the ability to inhibit NFκB activity is decreased. In other words, the DP148R gene of the attenuated ASF virus of the invention inhibits NFκB activity less than would a wild-type version of the DP148R gene. The ability to inhibit NFκB activity may be decreased by at least 50%, at least 60%, at least 70%, at least 80%, or at least 90%. In an embodiment the ability of the DP148R gene to inhibit NFκB activity may be abolished i.e. the DP148R gene does not inhibit NFκB activity. In that case, the activity of NFκB would be as high as if no DP148R gene were present. A decrease in inhibition means NFκB activity is higher than it would be with the wild-type version of the DP148R gene. NFκB activity may be twice as high as it would be with the wild type version of the DP148R gene, such as three times as high, four times as high, five times as high or six times as high.

NFκB is a transcription factor that activates transcription of various genes, controlling expression of interferon and pro-inflammatory cytokines as part of the host's innate immune system response to viral infection. Inhibition of NFκB activity means decreasing activation of expression of its target genes. The degree of activation of NFκB target gene expression may be measured using a luciferase reporter assay in which an NFκB-activatable promoter is linked to a luciferase reporter.

In an embodiment the activity of DP148R that may be disrupted is its ability to inhibit translocation of the p65 sub-unit of NFκB to the nucleus. In an embodiment, the invention provides an attenuated ASF virus in which the ability of the DP148R gene to inhibit translocation of the p65 sub-unit of NFκB to the nucleus is disrupted. Translocation of p65 to the nucleus can be measured by immunofluorescence of cells stimulated with NF-κB activators as described herein. The relative and absolute amounts of p65 that translocate to the nucleus may quantified by using differential cell lysis conditions known in the art to either break open the nucleus or leave it intact then quantify the amount of p65 using techniques known in the art such as Western blotting or mass spectrometry. For example, cells expressing a wild type or mutant DP148R protein may be stimulated to activate NF-κB then differentially lysed to produce whole cell lysate, a cytoplasmic fraction and a nuclear fraction. The amount of p65 in each fraction may then be quantified for the different conditions (wild type or mutant DP148R protein).

Disruption of the ability to inhibit translocation of p65 to the nucleus means that the ability to inhibit translocation of p65 to the nucleus is decreased. In other words, the version of the DP148R gene in the attenuated ASF virus of the invention inhibits translocation of p65 to the nucleus less than would a wild-type version of the DP148R gene. The ability to inhibit translocation of p65 to the nucleus may be decreased by at least 50%, at least 60%, at least 70%, at least 80%, or at least 90%. In an embodiment the ability of the DP148R gene to inhibit translocation of p65 to the nucleus may be abolished i.e. the DP148R gene does not inhibit translocation of p65 to the nucleus. In that case, as much p65 would translate to the nucleus as if no DP148R gene were present. A decrease in inhibition means the proportion of p65 that translocates to the nucleus is higher than it would be with the wild-type version of the DP148R gene. The proportion of p65 that translocates to the nucleus may be twice as high as it would be with the wild type version of the DP148R gene, such as three times as high, four times as high, five times as high or six times as high.

### Activity of EP153R

In an embodiment of the attenuated ASFV of the invention, EP153R activity may be disrupted.

In an embodiment the activity of EP153R that may be disrupted is its ability to mediate haemadsorption. In other words, the ability of EP153R to mediate haemadsorption may be decreased. The ability of EP153R to mediate haemadsorption may be decreased by at least 50, 60, 70, 80 or 90%. The ability of EP153R to mediate haemadsorption may be completely abolished.

In an embodiment the activity of EP153R that may be disrupted is its ability to inhibit caspase-3. In other words, the ability of EP153R to inhibit caspase-3 may be decreased. The ability of EP153R to inhibit caspase-3 may be decreased by at least 50, 60, 70, 80 or 90%. The ability of EP153R to inhibit caspase-3 may be completely abolished. Caspase-3 activity may be measured by assays known in the art, such as described by Hurtado et al. (Hurtado et al. 2004 Virology 326: 160-170). For example, the cleaved active caspase-3 fragment of 17 kDa and the inactive procaspase-3 protein of 34 kDa may be quantified using Western blot or mass spectrometry and compared to ascertain the degree of activation of caspase-3. Alternatively or additionally, the ability of caspase-3 in cell extract to cleave a fluorescent substrate may be measured using high performance liquid chromatography.

In an embodiment the activity of EP153R that may be disrupted is its ability to inhibit MHC-I expression. In other words, the ability of EP153R to inhibit MHC-I expression may be decreased. The ability of EP153R to inhibit MHC-I expression may be decreased by at least 50, 60, 70, 80 or 90%. The ability of EP153R to inhibit MHC-I expression may be completely abolished. MHC-I expression may be measured by assays known in the art, such as described by Hurtado et al. (Hurtado et al. 2011 Arch. Virol. 156(2): 219-234). For example, cell surface expression of MHC-I may be measured using antibody staining of non-permeabilised cells followed by flow cytometry.

### Activity of EP402R

In an embodiment of the attenuated ASFV of the invention, EP402R activity is disrupted.

In an embodiment the activity of EP402R that is disrupted is its ability to mediate haemadsorption. In other words, the ability of EP402R to mediate haemadsorption may be decreased. The ability of EP402R to mediate haemadsorption may be decreased by at least 50, 60, 70, 80 or 90%. The ability of EP402R to mediate haemadsorption may be completely abolished.

The activity of EP402R that is disrupted may be the ability of the EP402R protein to bind ligands via its extracellular N-terminal Ig-like domain (i.e. its ligand-binding domain). Thus in an embodiment the invention provides an attenuated ASF virus wherein the EP402R gene comprises one or more mutations that disrupt ligand binding by the EP402R protein. The ability of the EP402R protein to bind one or more ligands may be disrupted. The ability of EP402R to bind one or more ligands may be decreased by at least 50, 60, 70, 80 or 90%. The ability of the EP402R protein to bind one or more ligands may be completely abolished. The ability of the EP402R protein to bind one or more ligands may be completely abolished whilst retaining the ability to bind other ligands. In an embodiment, the invention provides an attenuated ASF virus wherein the EP402R gene comprises one or more mutations that disrupt ligand binding by the EP402R protein. Ligand binding may be measured using assays such as immunoprecipitation, surface plasmon resonance and/or isothermal calorimetry.

### Functional Version of a Gene

The attenuated ASFV of the invention comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

In some embodiments the attenuated ASFV of the invention may comprise a functional version of one or more of the following genes: MGF 110 3L, 6L, 7L, 8L, 10L, 11L and 12L; MGF 360 5L, 6L, 7L, 10L, 11L, 12L, 13L, 14L, 20R, 21R and 22R; and MGF 505 1R, 2R and 6R. Suitably, the attenuated ASFV comprises functional versions of two or more, such as three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one, or twenty two of the following genes: MGF 110 3L, 6L, 7L, 8L, 10L, 11L and 12L; MGF 360 5L, 6L, 7L, 10L, 11L, 12L, 13L, 14L, 20R, 21R and 22R; and MGF 505 1R, 2R and 6R. In an embodiment the attenuated ASFV comprises functional versions of all of the following genes: MGF 110 3L, 6L, 7L, 8L, 10L, 11L and 12L; MGF 360 5L, 6L, 7L, 10L, 11 L, 12L, 13L, 14L, 20R, 21R and 22R; and MGF 505 1R, 2R and 6R.

The expression “functional version” of a gene refers to a gene the expression and activity of which have not been disrupted. A functional version of a gene may not be mutated in a manner that disrupts gene expression or gene activity. A functional version of a gene may not comprise any mutations. The coding sequence of a functional version of a gene may be complete and uninterrupted. A functional version of a gene may be fully transcribed and translated.

A functional version of a gene may correspond to the gene in a wild-type ASFV isolate. A functional version of a gene may correspond to the gene in a virulent ASFV strain. The sequence of a functional version of a gene may be identical to the sequence of the gene in a wild-type ASFV isolate or virulent ASFV strain. The sequence of a functional version of a gene may be identical to the sequence of the gene in the wild-type ASFV isolate from which the attenuated ASFV of the invention is derived. A functional version of a gene may be a natural variant of the gene in a wild-type ASFV isolate.

A functional version of a gene may comprise mutations. However, the mutations should not disrupt the expression or activity of the gene. In other words, the mutations should not affect the function of the gene. A functional version of a gene may comprise one or more synonymous mutations (i.e. mutations which do not alter the amino acid sequence of the protein the gene encodes). A functional version of a gene may comprise one or more silent mutations, which may be synonymous or non-synonymous. A functional version of a gene may comprise deletions that do not disrupt the expression or activity of the gene. A functional version of a gene may comprise one or more single nucleotide polymorphisms (SNPs) that do not disrupt the expression or activity of the gene.

### Deletions

In the attenuated ASFV of the invention, expression and/or activity of genes may be disrupted by deletion. In other words, expression and/or activity of a gene may be disrupted by a mutation that is a deletion.

An attenuated ASFV of the invention may be made to lack a functional version of a gene by deletion. In other words, the mutation that causes the ASFV to lack a functional version of a gene may be a deletion.

“Deletion” means removal of part of the ASFV genome nucleotide sequence. The deletion may be continuous, or may comprise deletion of a plurality of sections of sequence. Deletion may disrupt gene expression and/or activity in any of the ways described herein. Deletion may cause the ASFV to lack a functional version of the gene in any of the ways described herein.

Deletion may disrupt transcription of the gene into mRNA. For example, deleting a promoter of a gene would disrupt transcription. Deletion may disrupt translation of mRNA into protein. For example, deleting a start codon would disrupt translation. Gene expression and/or activity may be disrupted by deleting non-coding sequence associated with the gene, such as a promoter.

Gene expression and/or activity may be disrupted by deleting coding sequence of the gene. The ASFV may be made to lack a functional version of the gene by deleting coding sequence of the gene. Deletion of coding sequence may be partial (i.e. part of the coding sequence is deleted). The deletion may, for example, remove at least 50, 60, 70, 80 or 90% of the coding sequence of the gene. The deletion may be complete, in which case 100% of the coding sequence of the gene is absent when compared to the corresponding genome of the wild-type isolate.

Deletion of coding sequence may be continuous, or may comprise deletion of a plurality of sections of coding sequence. The deletion should remove a sufficient amount of coding sequence such that deletion disrupts the expression and/or activity of the gene i.e. a functional gene product, such as a protein, is no longer produced from the gene. The expression “deletion of a gene” (such as “partially deleted”) refers to deletion of a sufficient amount of coding sequence such that expression and/or activity of the gene is disrupted. The amount of coding sequence required to be deleted to disrupt gene expression and/or activity may be very small. For example, deletion of just the start codon (ATG) may be sufficient to disrupt expression and/or activity of the gene to attenuate the virus.

Partial and full deletions of a gene can be made using known techniques in the art, such as conditional targeting via Cre-LoxP and Flp-FRT systems, or by inducing a double strand break (DSB) and repair using engineered nucleases such as meganucleases, zinc finger nucleases (ZFNs), transcription-activator like effector nucleases (TALENs) and Cas in CRISPR-Cas systems. The DSB repair can be exploited to introduce a desired mutation by providing a vector comprising the desired mutated nucleotide sequence within a sequence that is homologous to the sequences flanking either side of the DSB. This results in the desired mutation being inserted at the site of the DSB. Nucleases such as those above can be engineered to induce DSB at a specific site within the genome. For example, chimeric meganucleases can be readily generated by combining known protein units to recognise a target recognition sequence within a gene or genomic region of interest. ZFNs can also be designed to target specific sequences, for example combining zinc-finger units with known specificities to bind specific regions of DNA. TALENs are artificial restriction enzymes designed by fusing a nuclease domain to DNA-binding TALE (transcription activator-like effector) domains. TALE domains are tandem arrays of amino acid repeats that recognise a single nucleotide and can be designed to target a specific region of DNA. CRISPR-Cas systems consist of a Cas (CRISPR-associated protein) nuclease and a CRISPR (clustered regularly interspaced short palindromic repeat) RNA sequence that guides the Cas protein to recognise and cleave a specific strand of DNA complementary to the CRISPR sequence. Single-stranded guide RNA (sgRNA) can therefore be designed to bind to a specific region of DNA and guide the Cas to introduce a DSB. Accordingly, provided the nucleotide (e.g. DNA or cDNA) sequence of a gene is known, a known nuclease system can be utilised to introduce a partial or full deletion to the gene.

In an embodiment of the attenuated ASF virus of the present invention the genes DP148R, EP153R and EP402R may be at least partially deleted.

The DP148R gene may be at least partially deleted. The EP153R gene may be at least partially deleted. The EP402R gene may be at least partially deleted. The DP148R and EP153R genes may each be at least partially deleted. The DP148R, EP153R and EP402R genes may each be at least partially deleted.

In an embodiment of the attenuated ASF virus of the present invention the genes DP148R, EP153R and EP402R may be completely deleted.

The DP148R gene may be completely deleted. The EP153R gene may be completely deleted. The EP402R gene may be completely deleted. The DP148R and EP153R genes may each be completely deleted. The DP148R, EP153R and EP402R genes may each be completely deleted.

### Interruptions

The expression and/or activity of the genes may be disrupted by interruption of the gene. In other words, the mutation that disrupts expression and/or activity of a gene may be one that interrupts the gene.

In an embodiment of the attenuated ASFV of the present invention the genes DP148R, EP153R and EP402R may interrupted. The DP148R gene may be interrupted. The EP153R gene may be interrupted. The EP402R gene may be interrupted. The DP148R and EP153R genes may each be interrupted. The DP148R, EP153R and EP402R genes may each be interrupted.

“Interruption” means the mutation alters the coding sequence of the gene such that a functional gene product, such as a protein, is no longer produced. The term “interruption” may be used herein to refer to a mutation that interrupts a gene. The mutation(s) should interrupt the coding sequence in a manner such that expression and/or activity of the gene is disrupted i.e. a functional gene product, such as a protein, is no longer produced from the gene.

The interruption may entirely abolish gene product production. For example, where the gene product is a protein, the interruption may render the mRNA nonsensical, causing the mRNA to be degraded and the protein to not be translated, thereby abolishing protein production. The interruption may alter the gene product that is produced. The interruption may cause the gene to not be transcribed and/or translated.

The interruption may be a point mutation (i.e. substitution, insertion or deletion of a single nucleotide). An interruption may be a deletion. A gene may comprise multiple mutations that lead to interruption of the gene.

The interruption may be a frame shift mutation. A frame shift causes the codons downstream of the frame shift to be read as different amino acids. The protein produced may be non-functional.

The interruption may be mutation of a start codon. A start codon is typically ATG. Mutation of a start codon (e.g. point mutation of one, two or three of the nucleotides) means that translation will not start at that codon. Translation may begin at a subsequent start codon further downstream. If the subsequent start codon is in frame a version of the protein is produced that is N-terminally truncated and so may be non-functional. If the subsequent start codon is not in frame an entirely different or nonsense protein is produced, which would be non-functional. If there is no subsequent start codon, translation is entirely abolished and no protein is produced.

The interruption may be mutation of a stop codon (TAG, TAA or TGA). Mutation of a stop codon (also referred to as a nonstop mutation) causes continued translation of mRNA into a sequence that should not be translated. The resulting protein may be non-functional due to its excessive length.

### Amino Acid Changes

In an embodiment of the attenuated ASF virus of the invention, the EP402R gene comprises one or more mutations that change one or more amino acids in the ligand-binding domain of the EP402R protein. Suitably the EP402R gene comprises one or more mutations (such as two or more, three or more, four or more or five or more mutations) that change one or more amino acids (such as two or more, three or more, four or more or five or more amino acids) in the ligand-binding domain of the EP402R protein.

Changing of the one or more amino acids in the ligand-binding domain of the EP402R protein should disrupt expression and/or activity of the EP402R protein. In an embodiment, changing the one or more amino acids in the ligand-binding domain of the EP402R protein may disrupt haemadsorption mediated by the EP402R protein. In an embodiment, changing the one or more amino acids in the ligand-binding domain of the EP402R protein may disrupt ligand binding by the EP402R protein.

In an embodiment, the one or more mutations in the ligand-binding domain may be a deletion or an interruption as described herein. For example, deletion of part of the coding sequence for the ligand-binding domain of the EP402R protein would result in the absence of the encoded amino acids from the ligand-binding domain, which may disrupt ligand binding.

In an embodiment, one or more of the mutations may be a point mutation. Suitably one or more of the mutations may be point mutations that change a single amino acid into a different amino acid. Such an amino change may be referred to as a substitution. Changing even a single amino acid may disrupt the activity of the EP402R protein, such as ligand binding activity. For example, changing an amino acid that can be charged to an amino acid that can have the opposite charge may disrupt folding of the EP402R ligand-domain such that a binding pocket is deformed, which prevents ligand binding. Alternatively or additionally, substitution with an amino acid that can have an opposite charge may prevent electrostatic binding of a ligand. Another possibility is that changing an amino acid with a small side-chain to an amino acid with a bulky side-chain, or vice versa, may disrupt folding of the EP402R ligand-binding domain so that a binding pocket does not form which prevents ligand binding.

Thus in an embodiment the one or more changed amino acids in the ligand-binding domain may comprise a negatively charged amino acid that is changed to a positively charged amino acid. In an embodiment the one or more changed amino acids in the ligand-binding domain may comprise a positively charged amino acid that is changed to a negatively charged amino acid. Positively charged amino acids (i.e. amino acids that can have a positive charge) include histidine (H), lysine (K) and arginine (R). Negatively charged amino acids (i.e. amino acids that can have a negative charge) include aspartic acid (D) and glutamic acid (E).

In an embodiment the one or more changed amino acids in the ligand-binding domain may comprise an amino acid with a small side-chain that is changed to an amino acid with a bulky side-chain. In an embodiment the one or more changed amino acids in the ligand-binding domain may comprise an amino acid with a bulky side-chain that is changed to an amino acid with a small side-chain. Amino acids with a bulky side-chain include tryptophan (W). Amino acids with a small side-chain include glycine (G) and alanine (A).

In an embodiment the one or more changed amino acids in the ligand-binding domain may comprise an amino acid with a hydrophilic side-chain that is changed to an amino acid with a hydrophobic side-chain. In an embodiment the one or more changed amino acids in the ligand-binding domain may comprise an amino acid with a hydrophobic side-chain that is changed to an amino acid with a hydrophilic side-chain. Amino acids with a hydrophobic side-chain include alanine (A), valine (V), isoleucine (I), leucine (L), methionine (M), phenylalanine (F), tyrosine (Y) and tryptophan (VV). Amino acids with a hydrophilic side-chain include arginine (R), histidine (H), lysine (K), aspartic acid (D), glutamic acid (E), serine (S), threonine (T), asparagine (N) and glutamine (Q).

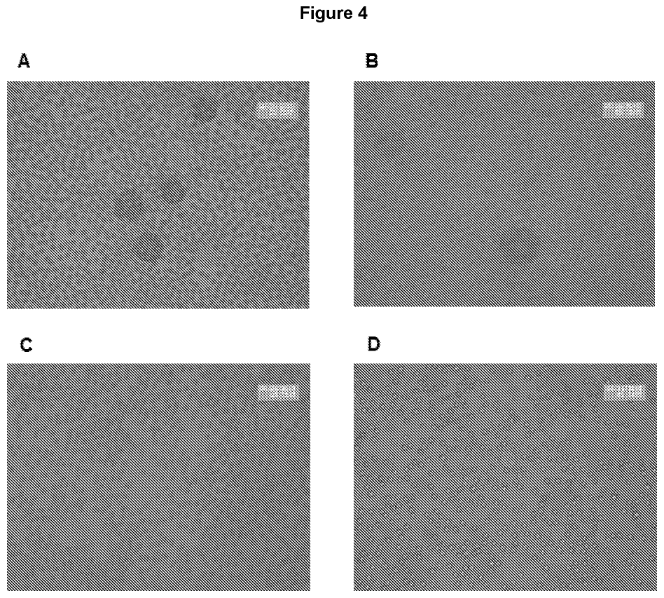

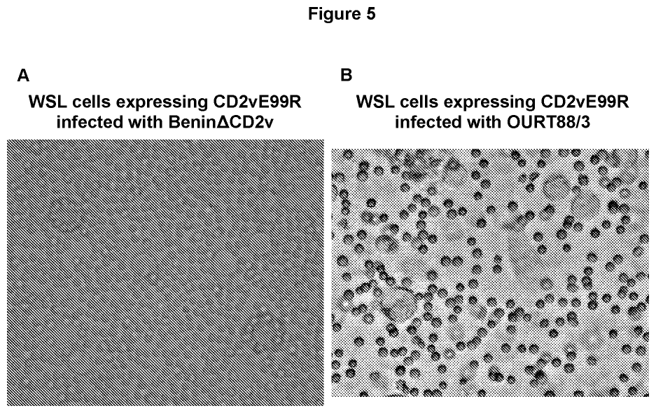

Changing amino acid E99 to R or amino acid Y102 to D disrupts the ability of EP402R protein from Benin 97/1 strain to mediate haemadsorption (see FIGS. 4, 5 and 6). Furthermore, changing the amino acid Q96 to R disrupts the ability of EP402R protein from Georgia 2007/1 strain to mediate haemadsorption (see FIG. 4C). Accordingly, in an embodiment the one or more mutations change an amino acid at a position in the EP402R protein which corresponds to E99 and/or Y102 of Benin 97/1 EP402R protein (SEQ ID NO. 11). In other words, the EP402R protein expressed by the attenuated ASF virus of the invention may be the EP402R protein from any ASF virus strain, and the amino acid that is changed corresponds to E99 and/or Y102 of the amino acid sequence of the EP402R protein from the Benin 97/1 strain, which is presented herein as SEQ ID NO. 11. The positions E99 and Y102 refer to amino acids glutamic acid and tyrosine at positions 99 and 102 respectively of the Benin 97/1 EP402R protein. In an embodiment the one or more mutations change the amino acid at position E99 and/or Y102 of the EP402R protein of the Benin 97/1 strain, or the corresponding position in the EP402R protein of any other ASFV strain. In an embodiment the one or more mutations change amino acids at positions in the EP402R protein which corresponds to E99 and Y102 of Benin 97/1 EP402R protein (SEQ ID NO. 11). In an embodiment the one or more mutations change an amino acid at a position in the EP402R protein corresponding to E99 of Benin 97/1 EP402R protein (SEQ ID NO. 11). In an embodiment the one or more mutations change an amino acid at a position in the EP402R protein which corresponds to Y102 of the Benin 97/1 EP402R protein (SEQ ID NO. 11).

Whether an amino acid corresponds to E99 and/or Y102 of the Benin 97/1 EP402R protein may be assessed by sequence alignment. For example, FIG. 3 shows an alignment of EP402R proteins from Benin, Mkuzi, Warmbaths, Tengnani, Warthog, Georgia and Malawi strains. The amino acid in the EP402R protein from each strain that corresponds to E99 of Benin 97/1 EP402R protein is the amino acid shown below the Benin E99 residue (highlighted in yellow). The amino acid in the EP402R protein from each strain that corresponds to Y102 of Benin 97/1 EP402R protein is the amino acid shown below the Benin Y102 residue (three amino acids to the right of the amino acids highlighted in yellow). The amino acids in the EP402R protein from the strains in FIG. 3 that corresponds to E99 and Y102 in Benin 97/1 EP402R are given in Table 4 below.

TABLE 4

| | Corresponding Amino acid | |
|---|---|---|
| Strain | E99 | Y102 |
| Mkuzi | E101 | E104 |
| Warmbaths | E98 | Y101 |
| Tengani | E100 | Y103 |
| Warthog | Q95 | Y98 |
| Georgia | Q96 | W99 |
| Malawi | Q97 | W100 |

Amino acids corresponding to E99 and Y102 of Benin 97/1 EP402R exist in EP402R proteins from strains other than those shown in FIG. 3 and listed in Table 4 above (such as other strains listed in Table 1 herein). The skilled person can readily determine using sequence alignment which amino acid in an EP402R protein from a given strain, such as a strain from Table 1, corresponds to E99 or Y102 of Benin 97/1 EP402R. The corresponding amino acid may be identical to E99 or Y102 of Benin 97/1 EP402R. For example, an amino acid corresponding to E99 of Benin 97/1 EP402R may also be an E (glutamic acid). The corresponding amino acid may be similar to E99 or Y102. Suitably the corresponding amino acid has the same charge as E99 or Y102. Suitably the corresponding amino acid to E99 is a Q (glutamine). Suitably the corresponding amino acid to Y102 is a W (tryptophan). The expression “corresponding to E99 and/or Y102 of Benin 97/1 EP402R protein” encompasses E99 and/or Y102D of Benin 97/1 EP402R protein.

In an embodiment the one or more mutations change an amino acid at position E99 and/or Y102 of Benin 97/1 EP402R protein (SEQ ID NO. 11). In an embodiment the one or more mutations change an amino acid at position E99 of Benin 97/1 EP402R protein (SEQ ID NO. 11). In an embodiment the one or more mutations change an amino acid at position Y102 of Benin 97/1 EP402R protein (SEQ ID NO. 11). In an embodiment the one or more mutations change an amino acid at position Q96 of Georgia 2007/1 EP402R protein (SEQ ID NO. 24).

In an embodiment the one or more mutations change an amino acid at a position in the EP402R protein corresponding to N16, 119, W21, Y76, E99, Y102 and/or N108 of Benin 97/1 EP402R protein (SEQ ID NO. 11). In an embodiment the one or more mutations change the amino acid at position N16, 119, W21, Y76, E99, Y102 and/or N108 of the EP402R protein of the Benin 97/1 strain, or the corresponding position in the EP402R protein of any other ASFV strain. In an embodiment the one or more mutations change an amino acid at a position in the EP402R protein corresponding to S15, W19, Q96, N104, and/or K108D of Georgia 2007/1 EP402R protein (SEQ ID NO. 24). In an embodiment the one or more mutations change the amino acid at position S15, W19, Q96, N104, and/or K108D of the EP402R protein of the Georgia 2007/1 EP402R protein (SEQ ID NO. 24), or the corresponding position in the EP402R protein of any other ASFV strain. In an embodiment the one or more mutations change an amino acid at a position in the EP402R protein corresponding to W20, Q112, N121 and/or R125 of N10 Genotype IX EP402R protein (SEQ ID NO. 27). In an embodiment the one or more mutations change the amino acid at position S15, W19, Q96, N104, and/or K108D of the EP402R protein of the N10 Genotype IX EP402R protein (SEQ ID NO. 27), or the corresponding position in the EP402R protein of any other ASFV strain. These amino acids are in the ligand-binding domain of the EP402R protein and are surface exposed.

Suitably, the mutation is selected from N16R, 119R, W21D, Y 76D, E99R, Y102D and/or N108R at a position corresponding to the position in the Benin 97/1 EP402R protein (SEQ ID NO. 11). Suitably, the mutation is a combination of E99R and N108R at the positions corresponding to the positions in the Georgia 2007/1 EP402R protein (SEQ ID NO. 11).

Suitably, the mutation is selected from S15R, W19D, Q96R, N104R and/or K108D at a position corresponding to the position in the Georgia 2007/1 EP402R protein (SEQ ID NO. 24). Suitably, the mutation is a combination of Q96R and N104R at the positions corresponding to the positions in the Georgia 2007/1 EP402R protein (SEQ ID NO. 24).

Suitably, the mutation is selected from W20D, Q112R, N121R and/or R125D at a position corresponding to the position in the N10 Genotype IX EP402R protein (SEQ ID NO. 27). Suitably, the mutation is a combination of Q112R and N121R at the positions corresponding to the positions in the N10 Genotype IX EP402R protein (SEQ ID NO. 27).

Suitably, where the attenuated ASFV is genotype I, the one or more mutations change an amino acid at a position in the EP402R protein corresponding to Y76, E99, Y102, and/or N108 of Benin 97/1 EP402R protein (SEQ ID NO. 11). Suitably, where the attenuated ASFV is genotype I, the mutation is selected from Y76D, E99R, Y102D, and/or N108R at a position corresponding to the position in the Benin 97/1 EP402R protein (SEQ ID NO. 11).

Suitably, where the attenuated ASFV is genotype II, the one or more mutations change an amino acid at a position in the EP402R protein corresponding to S15, W19, Q96, N104, and/or K108D of Georgia 2007/1 EP402R protein (SEQ ID NO. 24). Suitably, where there attenuated ASFV is genotype II, the mutation is selected from S15R, W19D, Q96R, N104R and/or K108D at a position corresponding to the position in the Georgia 2007/1 EP402R protein (SEQ ID NO. 24). Suitably, where the attenuated ASFV is genotype II, the mutation may be a combination of Q96R and N104R at the positions corresponding to the positions in the Georgia 2007/1 EP402R protein (SEQ ID NO. 24).

In an embodiment the amino acid at the position corresponding to E99 is changed to R and/or the amino acid at the position corresponding to Y102 is changed to D. In an embodiment the amino acid at the position corresponding to E99 is changed to R. In an embodiment the amino acid at the position corresponding to Y102 is changed to D. In an embodiment E99 of Benin 97/1 EP402R is changed to R and/or Y102 of Benin 97/1 EP402R is changed to D. In an embodiment E99 of Benin 97/1 EP402R is changed to R. In an embodiment Y102 of Benin 97/1 EP402R is changed to D. In an embodiment Q96 of Georgia 2007/1 EP402R is changed to R.

The amino acid sequence of the EP402R protein from Benin 97/1 strain comprising the E99R substitution is depicted below as SEQ ID No. 31.

Benin 97/1 EP402R E99R protein
SEQ ID No. 31
MIIIVIFLMCLKIVLNNIIIWSTLNQTVFLNNIFT
INDTYGGLFWNTYYDNNRSNFTYCGIAGNYCSCCG
HNISLYNTTNNCSLIIFPNNTEIFNRTYRLVYLDK
KINYTVKLLKSVDSPTITYNCTNSLITCKNNNGTN
VNIYLIINNTIVNDTNGDILNYYWNGNNNFTATCM
INNTISSLNETENINCTNPILKYQNYLSTLFYIII
FIVSGLIIGIFISIISVLSIRRKRKKHVEEIESPP
PSESNEEDISHDDTTSIHEPSPREPLLPKPYSRYQ
YNTPIYYMRPSTQPLNPFPLPKPCPPPKPCPPPKP
CPPPKPCPPPKPCSPPKPCRPPKPCPPPKPCPPPK
PCPPPKPCPPSKPCPSPESYSPPKPLPSIPLLPNI
PPLSTQNISLIHVDRII

In an embodiment the EP402R gene encodes a protein comprising the sequence of SEQ ID No. 31. Suitably, the EP402R gene encodes a protein comprising a sequence having at least 70%, at least 80%, at least 90% or at least 95% identity with SEQ ID No. 31, wherein amino acid 99 is R. Suitably, the EP402R gene encodes a protein consisting of the sequence of SEQ ID No. 31.

The amino acid sequence of the EP402R protein from Benin 97/1 strain comprising the Y102D substitution is depicted below as SEQ ID No. 32.

Benin 97/1 EP402R Y102D protein
SEQ ID No. 32
MIIIVIFLMCLKIVLNNIIIWSTLNQTVFLNNIFT
INDTYGGLFWNTYYDNNRSNFTYCGIAGNYCSCCG
HNISLYNTTNNCSLIIFPNNTEIFNRTYELVDLDK
KINYTVKLLKSVDSPTITYNCTNSLITCKNNNGTN
VNIYLIINNTIVNDTNGDILNYYWNGNNNFTATCM
INNTISSLNETENINCTNPILKYQNYLSTLFYIII
FIVSGLIIGIFISIISVLSIRRKRKKHVEEIESPP
PSESNEEDISHDDTTSIHEPSPREPLLPKPYSRYQ
YNTPIYYMRPSTQPLNPFPLPKPCPPPKPCPPPKP
CPPPKPCPPPKPCSPPKPCRPPKPCPPPKPCPPPK
PCPPPKPCPPSKPCPSPESYSPPKPLPSIPLLPNI
PPLSTQNISLIHVDRII

In an embodiment the EP402R gene encodes a protein comprising the sequence of SEQ ID No. 32. Suitably, the EP402R gene encodes a protein comprising a sequence having at least 70%, at least 80%, at least 90% or at least 95% identity with SEQ ID No. 32, wherein amino acid 102 is D. Suitably, the EP402R gene encodes a protein consisting of the sequence of SEQ ID No. 32.

The amino acid sequence of the EP402R protein from Georgia 2007/1 strain comprising the Q96R substitution is depicted below as SEQ ID No. 33.

Georgia 2007/1 EP402R Q96R protein
SEQ ID No. 33
MIILIFLIFSNIVLSIDYWVSFNKTIILDSNITND
NNDINGVSWNFFNNSFNTLATCGKAGNFCECSNYS
TSIYNITNNCSLTIFPHNDVFDTTYRVVWNQIINY
TIKLLTPATPPNITYNCTNFLITCKKNNGTNTNIY
LNINDTFVKYTNESILEYNWNNSNINNFTATCIIN
NTISTSNETTLINCTYLTLSSNYFYTFFKLYYIPL
SIIIGITISILLISIITFLSLRKRKKHVEEIESPP
PESNEEEQCQHDDTTSIHEPSPREPLLPKPYSRYQ
YNTPIYYMRPSTQPLNPFPLPKPCPPPKPCPPPKP
CPPPKPCPSAESYSPPKPLPSIPLLPNIPPLSTQN
ISLIHVDRII

In an embodiment the EP402R gene encodes a protein comprising the sequence of SEQ ID No. 33. Suitably, the EP402R gene encodes a protein comprising a sequence having at least 70%, at least 80%, at least 90% or at least 95% identity with SEQ ID No. 33, wherein amino acid 96 is R. Suitably, the EP402R gene encodes a protein consisting of the sequence of SEQ ID No. 33.In an embodiment the EP402R gene encodes a protein comprising a sequence selected from SEQ ID No. 31, 32 or 33.

### Mutation Combinations

The mutations that disrupt gene expression and/or activity described herein may be combined in an attenuated ASFV of the invention. In other words, each of the DP148R, EP153R and EP402R genes in an attenuated ASFV of the invention may be disrupted by the same type of mutation as any of the other genes or by a different type of mutation as any of the other genes.

For example, in an attenuated ASFV of the invention, DP148R may be disrupted by mutation of a promoter sequence, EP153R may be disrupted by complete deletion and EP402R may be disrupted by an amino acid change in its ligand-binding domain. As an alternative example, DP148R may be disrupted by partial deletion, EP153R may be disrupted by interruption and EP402R may be disrupted by complete deletion.

In an embodiment the invention provides an attenuated ASFV in which

the DP148R gene and EP153R gene are each completely deleted, and

the EP402R gene comprises a mutation that changes an amino acid at a position in the EP402R protein which corresponds to E99 of Benin 97/1 EP402R protein (SEQ ID No. 11) to R;

and which comprises a functional version of one or more of the following genes:
MGF 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

The attenuated ASFV may comprise a functional version of one or more of the MGF genes as described herein.

In an embodiment the invention provides an attenuated ASFV in which
the DP148R gene and EP153R gene are each completely deleted, and
the EP402R gene comprises a mutation that changes an amino acid at a position in
the EP402R protein which corresponds to E99 of Benin 97/1 EP402R protein (SEQ ID No. 11) to R;
   and which comprises a functional version of one or more of the following genes:
MGF 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

The attenuated ASFV may comprise a functional version of one or more of the MGF genes as described herein.

In an embodiment the invention provides an attenuated ASFV in which
the DP148R gene and EP153R gene are each completely deleted, and
the EP402R gene comprises a mutation that changes an amino acid at a position in
the EP402R protein equivalent to Y102 of Benin 97/1 EP402R protein (SEQ ID No. 11) to D;
   and which comprises a functional version of one or more of the following genes:
MGF 110 3L, 6L, 7L, 8L, 10L, 11L and 12L,
MGF 360 5L, 6L, 7L, 10L, 11 L, 12L, 13L, 14L, 20R, 21R and 22R, and
MGF 505 1R, 2R and 6R.

The attenuated ASFV may comprise a functional version of one or more of the MGF genes as described herein.

### Vaccine/Pharmaceutical Composition

The present invention also provides a vaccine comprising an attenuated ASF virus of the invention.

The term “vaccine” as used herein refers to a preparation which, when administered to a subject, induces or stimulates a protective immune response. A vaccine can render an organism immune to a particular disease, in the present case ASF. The vaccine of the present invention thus induces an immune response in a subject which is protective against subsequent ASF virus challenge. A vaccine comprising an attenuated ASFV of the invention may be capable of inducing a cross-protective immune response against a plurality of ASF virus genotypes. In an embodiment a vaccine comprising an attenuated ASFV of the invention of a single genotype may be capable of inducing a cross-protective immune response against a plurality of ASF virus genotypes.

The vaccine may comprise a plurality of attenuated ASF viruses. The plurality of attenuated ASF viruses may correspond to a plurality of different isolates, for example, different isolates of high or unknown virulence. Such a vaccine may be capable of inducing a cross-protective immune response against a plurality of ASF virus genotypes.

The vaccine may be useful in preventing African Swine Fever. Accordingly the invention provides a vaccine of the invention for use in treating and/or preventing African Swine Fever in a subject.

The present invention also provides a pharmaceutical composition which comprises one or more attenuated ASF virus(es) of the invention. The pharmaceutical composition may be used for treating African Swine Fever.

The vaccine or pharmaceutical composition may comprise one or more attenuated ASF virus(es) of the invention and optionally one or more adjuvants, excipients, carriers and diluents. The choice of pharmaceutical excipient, carrier or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s) and other carrier agents. The pharmaceutical compositions typically should be sterile and stable under the conditions of manufacture and storage. Formulations for parenteral administration include, but are not limited to, suspensions, solutions, emulsions in oily or aqueous vehicles, pastes, and implantable sustained-release or biodegradable formulations. Sterile injectable formulations may be prepared using a non-toxic parenterally acceptable diluent or solvent. A pharmaceutical composition of the present invention may include pharmaceutically acceptable dispersing agents, wetting agents, suspending agents, isotonic agents, coatings, antibacterial and antifungal agents, carriers, excipients, salts, or stabilizers which are nontoxic to the subjects at the dosages and concentrations employed. Preferably, such a composition can further comprise a pharmaceutically acceptable carrier or excipient for use in the treatment of disease that that is compatible with a given method and/or site of administration, for instance for parenteral (e.g. sub-cutaneous, intradermal, or intravenous injection) or intrathecal administration.

The vaccine or pharmaceutical composition may comprise one or more attenuated ASF virus(es) of the invention in an effective amount.

In an embodiment the invention provides an attenuated ASF virus of the invention which when administered to a subject induces an immune response which is protective against subsequent challenge with virulent ASF virus. In an embodiment the invention provides an attenuated ASF virus of the invention which when administered to a subject induces an immune response which is protective against subsequent challenge with virulent ASF virus of a different genotype to the attenuated ASF virus of the vaccine.

### Methods of Prevention/Treatment

The present invention also provides a method of preventing and/or treating ASF in a subject by administration to the subject of an effective amount of an attenuated virus, vaccine, or pharmaceutical composition of the invention.

The term “preventing” is intended to refer to averting, delaying, impeding or hindering the contraction of ASF. The vaccine may, for example, prevent or reduce the likelihood of an infectious ASFV entering a cell.

The term “treating” is intended to refer to reducing or alleviating at least one symptom of an existing ASF infection.

The subject may be any animal which is susceptible to ASF infection. ASF susceptible animals include domestic pigs, warthogs, bush pigs and ticks.

The subject vaccinated according to the present invention may be a domestic pig.

### Administration

The vaccine of the invention may be administered by any convenient route, such as by intramuscular injection. Other suitable routes of administration include intranasal, oral, subcutaneous, transdermal and vaginal (e.g. during artificial insemination). In one embodiment, oral administration comprises adding the vaccine to animal feed or drinking water. In another embodiment, the vaccine may be added to bait for a wild animal, for example bait suitable for wild boar, wild pigs, bushpigs or warthogs.

The dose for pig immunisation may be less than 10⁴ HAD₅₀ or TCID₅₀ per pig. For example the dose may be between 10²-10³ HAD₅₀ or TCID₅₀ The dose may be about 10² HAD₅₀ or TCID₅₀ per pig. The dose may be determined by a veterinary practitioner within the scope of sound veterinary judgment.

The vaccine may be administered following a prime-boost regime. For example, after the first inoculation, the subjects may receive a second boosting administration some time (such as about 7, 14, 21 or 28 days) later. Typically the boosting administration is at a higher dose than the priming administration. The boosting dose may be about 10², 10³ or 10⁴ HAD₅₀ or TCID₅₀ of the recombinant attenuated virus per pig.

### Method for Preparing an Attenuated Virus

The present invention also provides a method of attenuating an ASF virus which comprises the step of disrupting the expression and/or activity of the genes DP148R, EP153R and EP402R.

Disruption of gene expression and/or activity may be achieved by mutating the ASFV genome in any of the ways described herein.

The DP148R, EP153R and/or EP402R genes may be partially or completely deleted.

The DP148R gene may be at least partially or completely deleted. The EP153R gene may be at least partially or completely deleted. The EP402R gene may be at least partially or completely deleted. The DP148R and EP153R genes may be at least partially or completely deleted. The DP148R, EP153R and EP402R genes may each be partially or completely deleted. The coding sequences of one or more (such as two or more, or all three) of the disrupted genes may be at least partially or completely deleted.

The DP148R, EP153R and/or EP402R genes may be interrupted.

The DP148R gene may be interrupted. The EP153R gene may be interrupted. The EP402R gene may be interrupted. The DP148R and EP153R genes may be interrupted. The DP148R, EP153R and EP402R genes may each be interrupted. The coding sequences of one or more (such as two or more, or all three) of the disrupted genes may be interrupted.

In an embodiment the method of attenuating an ASF virus comprises disrupting the ability of the EP402R gene to mediate haemadsorption.

In an embodiment the method of attenuating an ASF virus comprises comprising introducing one or more mutations in the EP402R gene that disrupt ligand binding by the EP402R protein.

In an embodiment the method of attenuating an ASF virus comprises introducing one or more mutations in the EP402R gene that change one or more amino acids in the ligand-binding domain of the EP402R protein. The mutation may be one or more of the mutation as described herein.

In an embodiment the method of attenuating an ASF virus comprises changing the amino acid at position R96, E99 and/or Y102 of the EP402R protein.

In an embodiment the method of attenuating an ASF virus comprises changing the amino acid at position R96 to Q, the amino acid at position E99 to R and/or the amino acid at position Y102 to D. Suitably the method comprises changing the amino acid at position R96 to Q. Suitably the method comprises changing the amino acid at position E99 to R. Suitably the method comprises changing the amino acid at position Y102 to D.

In an embodiment the method of attenuating an ASF virus comprises completely deleting the DP148R gene, completely deleting the EP153R gene and introducing a mutation in the EP402R gene that changes the amino acid at position R96 of the EP402R protein to Q. In an embodiment the method of attenuating an ASF virus comprises completely deleting the DP148R gene, completely deleting the EP153R gene and introducing a mutation in the EP402R gene that changes the amino acid at position E99 of the EP402R protein to R. In an embodiment the method of attenuating an ASF virus comprises completely deleting the DP148R gene, completely deleting the EP153R gene and introducing a mutation in the EP402R gene that changes the amino acid at position Y102 of the EP402R protein to D.

Methods for mutation of viral genes are known in the art. In particular, methods for deletion of viral genes are known in the art. For example, homologous recombination may be used, in which a transfer vector is created in which the relevant gene(s) are missing and used to transfect virus-infected cells. Recombinant viruses expressing the new portion of sequence may then be selected. Similar procedures may be used in order to interrupt gene expression, for example by deletion of the ATG start codon.

“Retaining the function” of a gene means that expression and activity of the gene is not affected during the attenuation process. The resultant attenuated virus should express a functional version of the gene. Suitably, the genes the function of which is to be retained are unaltered by the method of attenuation. Suitably, the sequences of the genes the function of which is to be retained are unaltered by the method of attenuation.

In some embodiments, the method of attenuating an ASF virus may comprise retaining the function of one or more of the following genes: MGF 110 3L, 6L, 7L, 8L, 10L, 11L and 12L; MGF 360 5L, 6L, 7L, 10L, 11L, 12L, 13L, 14L, 20R, 21R and 22R; and MGF 505 1R, 2R and 6R. Suitably, the function of two or more, such as three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one, or twenty two of the following genes are retained: MGF 110 3L, 6L, 7L, 8L, 10L, 11L and 12L; MGF 360 5L, 6L, 7L, 10L, 11L, 12L, 13L, 14L, 20R, 21R and 22R; and MGF 505 1R, 2R and 6R. In an embodiment the method of attenuating an ASF virus may comprise retaining the function of all of the following genes: MGF 110 3L, 6L, 7L, 8L, 10L, 11L and 12L; MGF 360 5L, 6L, 7L, 10L, 11 L, 12L, 13L, 14L, 20R, 21R and 22R; and MGF 505 1R, 2R and 6R.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Identification of EP402R/CD2v Mutants that Reduce HAD

Mutations were made in African swine fever virus (ASFV) Benin isolate EP402R protein (CD2v) and tested for their effect on haemadsorption (HAD).

A model of the extracellular, N-terminal, IgG-like, ligand-binding domain of CD2v was generated and used to predict the functional amino acid residues involved in binding of CD2v to its ligand. These residues were individually mutated to generate a set of mutant CD2v proteins.

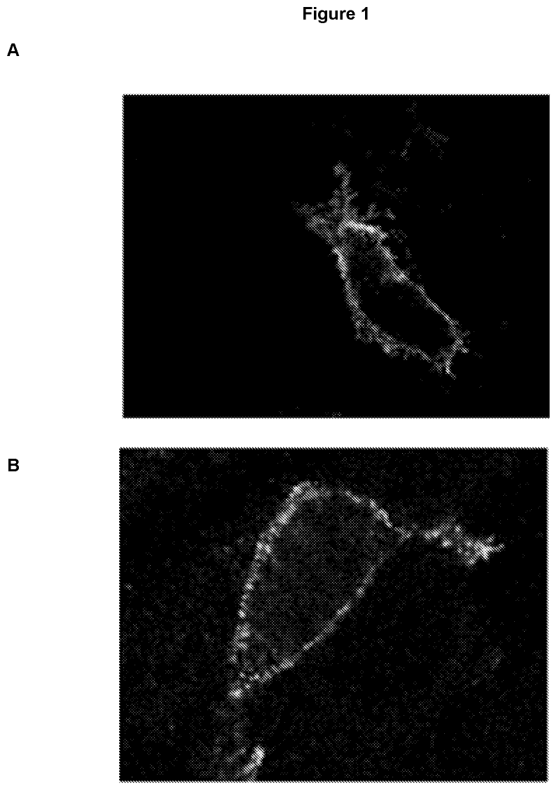

Vero cells were infected with modified vaccinia virus Ankara expressing T7RNA polymerase and transfected with plasmids (pcDNA3) expressing wild-type or mutant CD2v full-length proteins with a C-terminal HA epitope tag. Pig red blood cells were added and cells observed for attachment of red blood cells to the surface. Expression of the wild-type or mutant CD2v proteins was confirmed both by confocal microscopy using permeabilised cells and Western blotting using an antibody recognising the HA tag and a secondary antibody. Cell surface expression of the wild-type or mutant CD2v was also confirmed by staining non-permeabilised cells with sera from pigs immunised with attenuated ASFV containing a wild-type CD2v gene and a secondary antibody (FIG. 1).

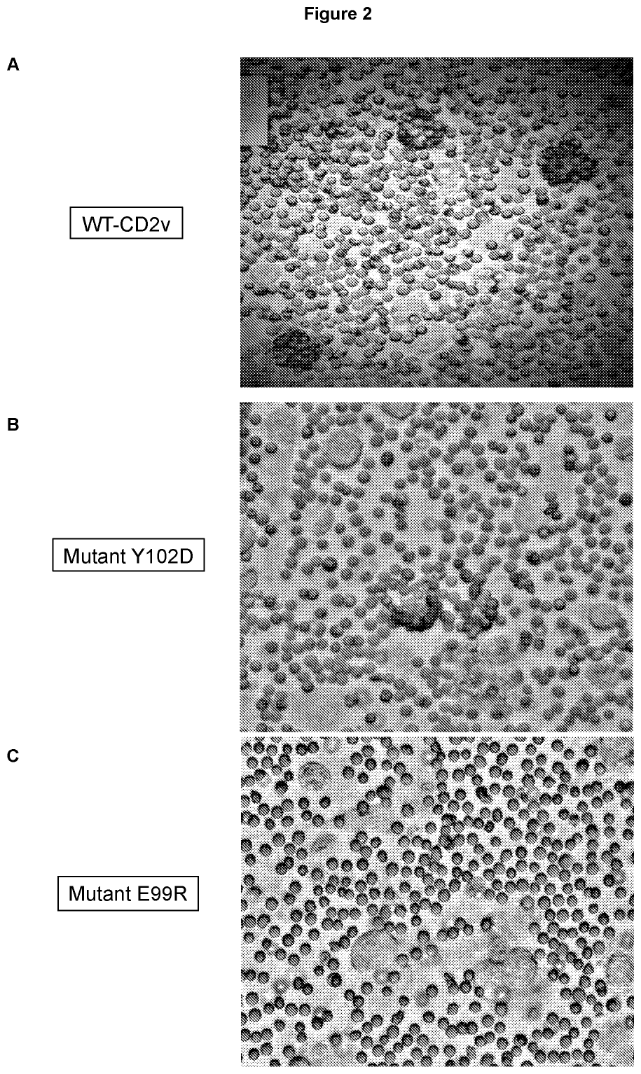

Mutation of residues E99 or Y102 abrogated HAD. FIG. 2 shows exemplary images from the HAD assay. FIG. 2A shows cells transfected with a plasmid expressing wild-type Benin CD2v. HAD of red blood cells is observed around three cells. FIG. 2B shows cells expressing CD2v with the Y102 residue mutated to D. Partial HAD is observed around one cell. FIG. 2C shows cells expressing CD2v with residue E99 mutated to R. No HAD is observed.

CD2v residue E99 is strongly conserved in ASFV, as shown by an alignment of the amino acid sequence of CD2v ligand-binding domain from different ASFV isolates of varying genotypes (FIG. 3). The equivalent residue in other isolates (highlighted in yellow in FIG. 3) is either identical (E) or has the same charge (Q).

The equivalent residue to Benin CD2v E99 in Georgia CD2v, Q96, was mutated to R and the ability of the mutant protein to induce HAD was tested using the HAD assay described above. Mutation of Q96 in Georgia CD2v abrogated HAD. FIG. 4 shows exemplary images from the HAD assay of Vero cells with pig red blood cells. FIG. 4A shows Vero cells transfected with a plasmid expressing wild-type Benin CD2v. HAD of red blood cells is observed around four cells. FIG. 4B shows Vero cells expressing wild-type Georgia CD2v. HAD is observed around two cells. FIG. 4C shows Vero cells expressing Georgia CD2v with residue Q96 mutated to R. No HAD is observed. FIG. 4D shows non-transfected Vero cells. No HAD is observed.

Using the same assay, the following mutations were determined to impact HAD in Benin: N16R, I 19R, W21D, Y 76D, E99R, Y102D. In addition, a combination of E99R +N108R was determined to impact HAD in Benin.

The following mutations were determined to impact HAD in Georgia: S15R, W19D, Q96R, N104R, and K108D. In addition, combinations of S15R +W19D and Q96R +N104R were determined to impact HAD in Georgia.

The following mutations were determined to impact HAD in N10 Genotype IX: W20D, R125D, Q112R +N121R.

### Example 2—Generation of ASFV Lacking Functional DP148R, EP153R and EP402R

The ability to induce HAD could be restored by infecting WSL cells expressing the CD2vE99R mutant with BeninΔCD2v virus (FIG. 4A).

However, HAD could not be restored by infecting WSL cells expressing the CD2vE99R mutant with OURT88/3 virus (FIG. 4B). OURT88/3 has frame-shift interruptions in the EP153R and EP402R/CD2v genes, which suggests a role for EP153R in rescuing HAD caused by the CD2vE99R mutant.

Based on these results, two gene deleted Benin viruses were constructed using as backbone the attenuated BeninΔDP148R virus. One virus had in addition to DP148R further deletions of EP153R and EP402R (BeninΔDP148RΔEP153RΔCD2v) and the second had EP153R deleted and the wild-type EP402R gene replaced with the gene expressing CD2vE99R mutant (BeninΔDP148RΔEP153R-CD2vE99R).

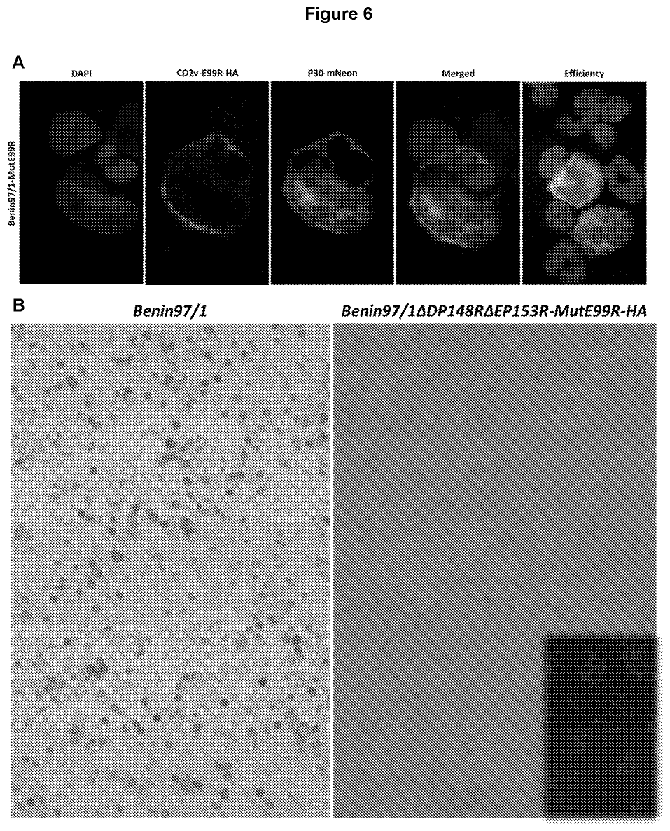

The Benin CD2vE99R mutant protein was expressed at the cell surface, as shown in FIG. 6A. Porcine bone marrow cells were infected with BeninΔDP148RΔEP153R-CD2vE99R, fixed and permeabilised at 8 hours post-infection then stained with an antibody which recognises the HA-tag fused in frame with the CD2vE99R gene and secondary antibody. The CD2vE99R is shown in red on the cell surface. The mNeon green was inserted in place of the EP153R gene and is shown in green. Nuclear DNA and virus cytoplasmic factories are stained blue. The panel on the right shows a larger field to indicate numbers of infected cells.

Cells infected with these viruses did not induce HAD. FIG. 6B shows porcine bone marrow cells infected with wild type Benin97/1 virus (left panel) or BeninΔDP148RΔEP153R-CD2vE99R (right panel). Red blood cells are observed forming rosettes around the Benin97/1 infected cells (left panel) but not around the cells infected with virus expressing mutant CD2vE99R (right panel). The inset bottom right shows cells infected with BeninΔDP148RΔEP153R-CD2vE99R expressing mNeon protein to confirm the cells were infected.

### Example 3—DP148R Inhibits Activation of NF-κB by Inhibiting Nuclear Translocation of the p65 Sub-Unit

HEK293T cells were transfected with an NF-κB dependent luciferase reporter plasmid and either a plasmid encoding the DP148R gene or an empty plasmid (pcDNA3) as a negative control. Twenty four hours post transfection the cells were treated with IL-1β (FIG. 7A) or TNF-α (FIG. 7B) for eight hours to induce NF-κB activation. Following cell lysis, luciferase activity was measured. FIG. 7A and FIG. 7B show that in the presence of DP148R, luciferase values were significantly lower than those obtained from cells transfected with pcDNA3. This indicates that DP148R is able to inhibit NF-KB mediated signalling.

In a pair of alternative experiments, NF-κB activation was instead induced by overexpression of the two main kinases of the NF-κB signalling pathway, namely IKKα (FIG. 7C) and IKKβ (FIG. 7D). FIG. 7C and FIG. 7D show that, again, in the presence of DP148R, luciferase values were significantly lower than those obtained from cells transfected with pcDNA3, indicating that DP148R is able to inhibit the NF-κB pathway at the level of the kinases activation.

To further elucidate the mechanism by which DP148R inhibits NF-κB activation, the effect of DP148R on nuclear translocation of the p65 sub-unit of NF-κB was investigated. HeLa cells were transfected with a plasmid expressing HA-tagged DP148R and were then stimulated with TNF-alpha or IL-β to activate NF-κB. Unstimulated cells were used as a negative control. Cells were fixed and permeabilised then stained for HA-tagged DP148R using an anti-HA tag antibody and an appropriate secondary antibody. The p65 subunit of NF-κB was stained with a primary antibody and secondary antibody. FIG. 8 shows immunofluorescence images of the cells. HA-tagged DP148R is shown in red, p65 is shown in green and nuclei are shown in blue. In cells not expressing DP148R, in unstimulated cells p65 remains in the cytoplasm but translocates into the nucleus following stimulation with TNF-alpha or IL-1beta. In contrast, in cells expressing DP148R, p65 nuclear translocation is not observed upon stimulation and lower amounts of p65 are detected in the cytoplasm. The results show that DP148R is an efficient inhibitor of NF-κB which would inhibit activation of genes dependent on NF-κB. These include for example proinflammatory cytokines and chemokines.

### Example 4—Lack of Functional DP148R, EP153R and EP402R Attenuates ASFV and Induces Protection Against Challenge

### Vaccination Experimental Protocol

Groups of large white/landrace pigs were immunised intramuscularly with 10⁴ TCID50 BeninΔDP148RΔEP153RΔCD2v (Group A—eight pigs) and BeninΔDP148RΔEP153R-CD2vE99R (Group B—six pigs).

Pigs were boosted on day 21 (10⁴ TCID50/m1) and day 28 (10⁶ TCID50/m1) post-immunisation and then challenged with 10³ TCID50/m1 parental virulent Benin 97/1 strain on day 45 post-immunisation, in parallel with three non-immunised pigs (control Group E).

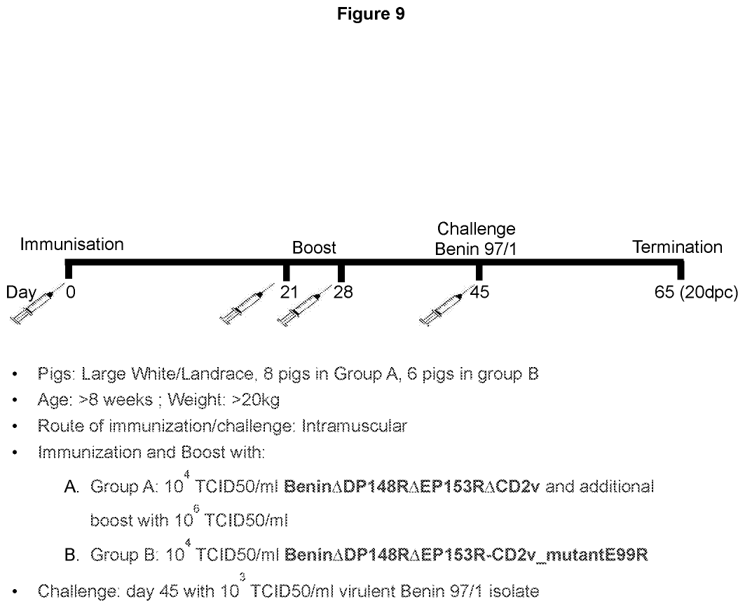

The experimental protocol is depicted in FIG. 9.

### Temperature

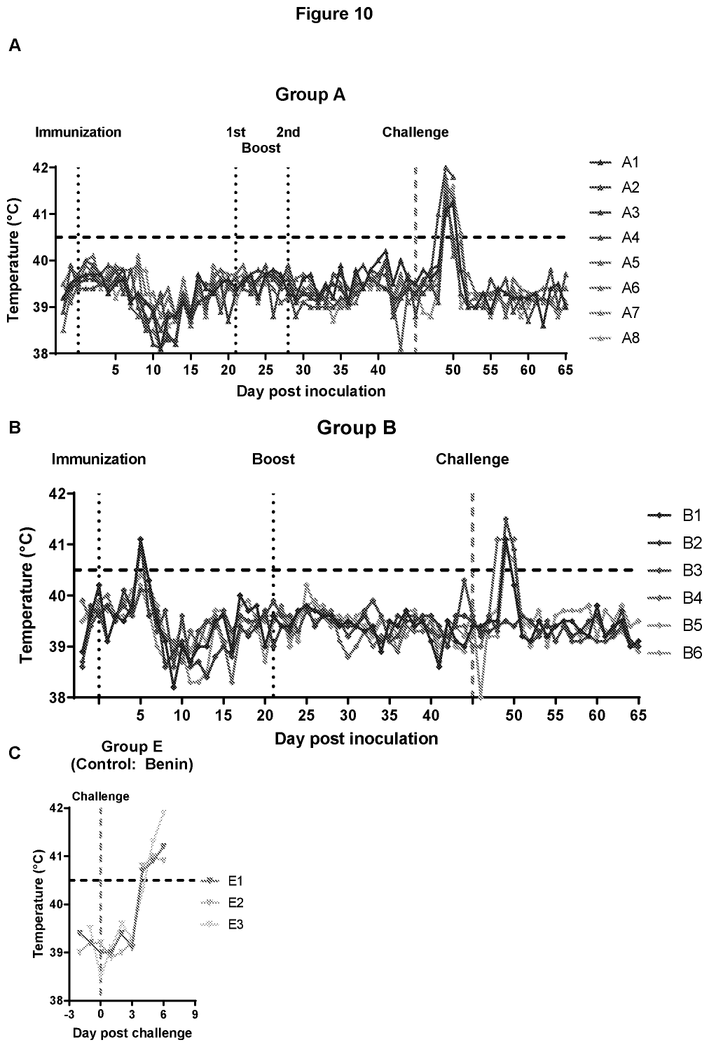

Rectal temperatures were recorded daily for all pigs in Group A and B and the control Group E (FIG. 10). In Group A, no increase in temperature was observed before challenge. After challenge an increase in temperature at or above 41° C. was observed in one pig at day 3 post-challenge and in the remaining pigs at day 4 post-challenge. Two pigs (A3 and A4) reached the humane end-point at day 5 post-challenge and were culled. The remaining pigs had an increased temperature above 40.5° C. for two days in total, except for pig A7 in which a temperature of 40.6° C. persisted for a 3^{rd} day. All then survived to the end of the experiment at day 20 post-challenge without further increase in temperature (FIG. 10A).

Three pigs in Group B had an increase in temperature above 40.5° C. at day 5 post-immunisation. One pig had a temperature of 41.1° C. by day 3 post-challenge and this was maintained a further 2 days when the animal was culled at the humane endpoint. From the other pigs all survived, one had a single day temperature above 40.5° C. at day 4 post-challenge, three had 2 days increase temperature (days 4 and 5) and one had no increased temperature. All survivors had no further temperature increase until the end of the experiment (day 20 post-challenge) (FIG. 10B).

Control pigs had temperatures above 40.5° C. from day 4 post-challenge and were culled at the humane endpoint on day 6 post-challenge (FIG. 10C).

### Clinical Scores

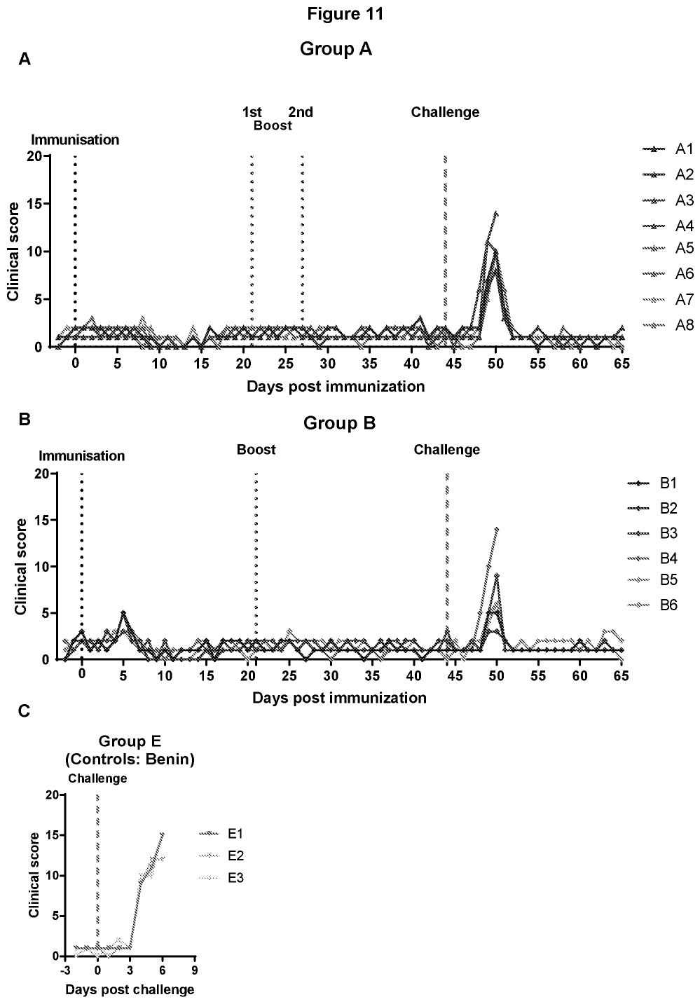

Clinical scores, including increased temperature and other signs such as lethargy and loss of appetite were collected daily (FIG. 11). In Group A no signs were observed before challenge. After challenge clinical scores were observed in all pigs coincident with increased temperatures and reaching higher levels in the two pigs that were culled at the humane endpoint (FIG. 11A).

In Group B mild signs were observed in pigs post-immunization coincident with temperature increases. After challenge mild signs were observed except for the pig which was culled at the humane endpoint and the pig which did not show an increase in temperature which had no clinical signs (FIG. 11B).

### Viremia

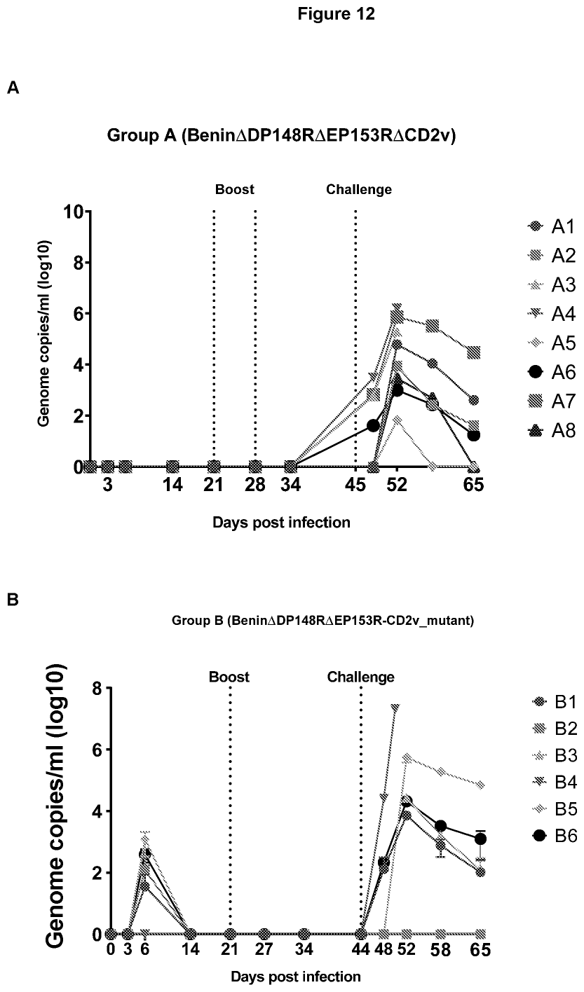

Levels of virus genome in blood were measured by qPCR (FIG. 12). No virus genome was detected in blood from pigs in Group A before challenge. After challenge variable levels of genome were detected from day 3 or 4 post-challenge. Maximum levels were detected in the pigs which were culled (˜10e6 genome copies per ml). In other pigs the maximum levels detected varied between ˜10e2 per ml and 10e5.5 per ml. Levels of virus genome decreased until termination at day 20 post-challenge becoming undetectable in 2 pigs and reduced to 10e2.6 in others except 1 pig in which levels remained at 10e4.5 at termination (FIG. 12A).

In group B low levels of virus genome were detected in blood from 3 pigs at day 6 post-immunization. After challenge virus genome was detected at low levels (below 10e4 genome copies per ml) from day 7 post-challenge (FIG. 12B).

### Antibody Response

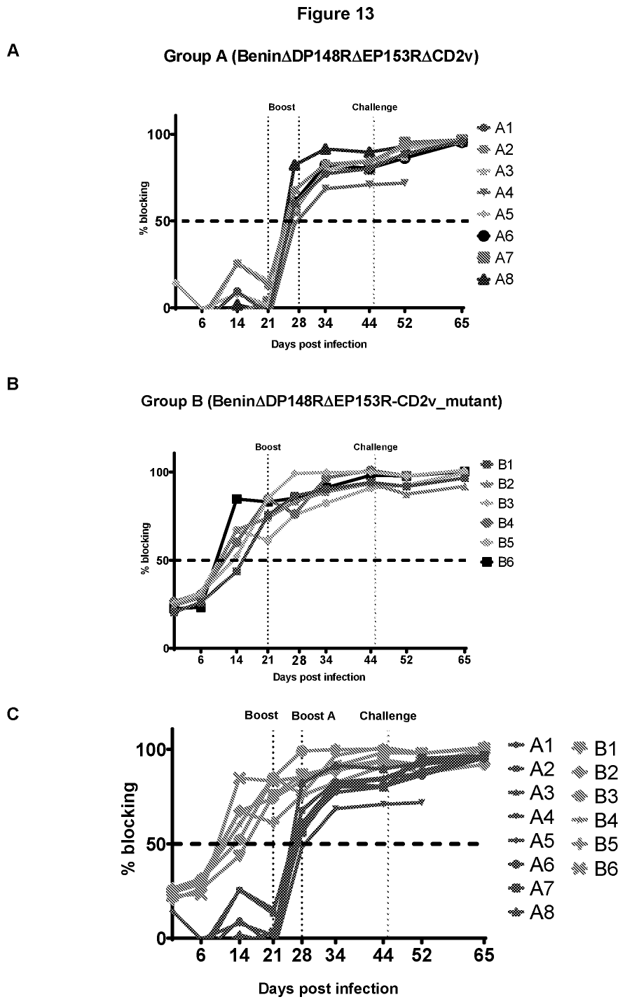

ASFV specific antibody responses were measured using a commercially available competitive ELISA based on the VP72 (B646L) major capsid protein (FIG. 13). In Group A antibody responses were first detected at day 27 post-immunization after the first boost and just before the second boost. Levels were maintained for the rest of the experiment (FIG. 13A).

In pigs in Group B antibody responses were detected by day 14 (1 week before the boost) and increased maintaining a plateau after day 21 to 28 (FIG. 13B).

### Cell-Mediated Immune Response

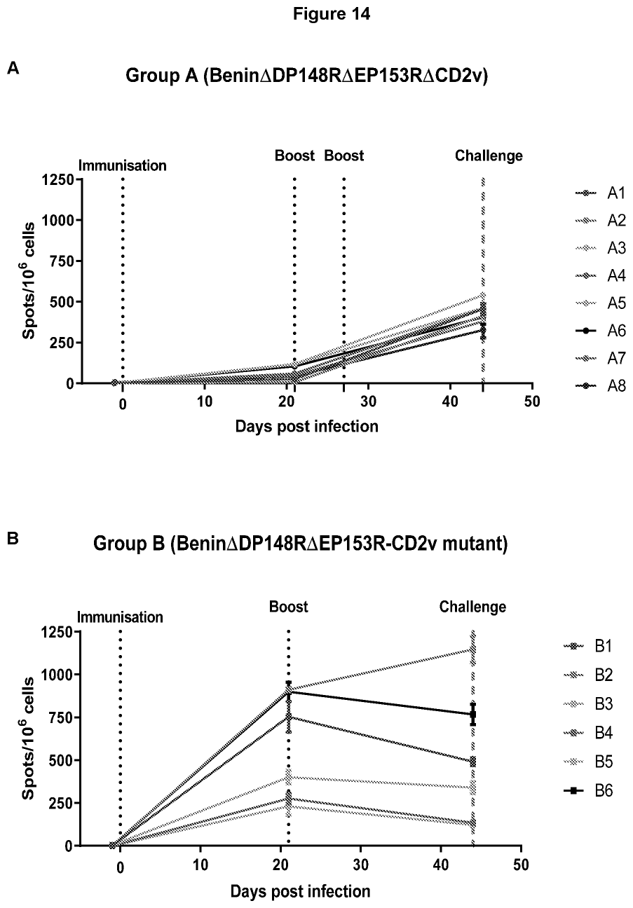

Peripheral blood mononuclear cells were collected pre-immunization, boost and challenge from pigs in Groups A and B and stimulated with ASFV Benin virus. Numbers of IFN gamma producing cells were measured by Elispot assay (FIG. 14). In Group A low levels of IFN gamma producing cells were induced after immunisation and before boost and these increased uniformly in all pigs before challenge (FIG. 14A). In Group B responses were more variable between pigs and pigs had had higher numbers of IFN gamma producing cells before boost. However at the time of challenge these numbers had decreased slightly in all except 1 pig. The pig which was culled at the humane end point (B4) had a lower number of IFN gamma producing cells although similar in number to a pig which survive challenge (B5) (FIG. 14B).

### Scoring of Macroscopic Lesions at Post-Mortem

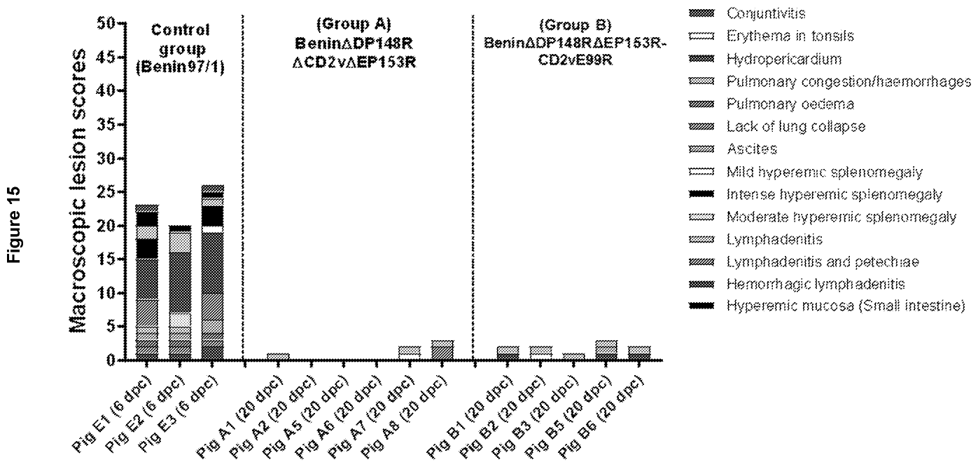

Control non-immune pigs (Group E) showed lesions typical of acute ASFV including enlarged and haemmorhagic lymph nodes and spleen. Those pigs which survived challenge had few, if any, ASFV specific lesions (FIG. 15). The pigs culled at humane end points showed higher scores but less than the control pigs (data not shown).

### Viremia Comparison

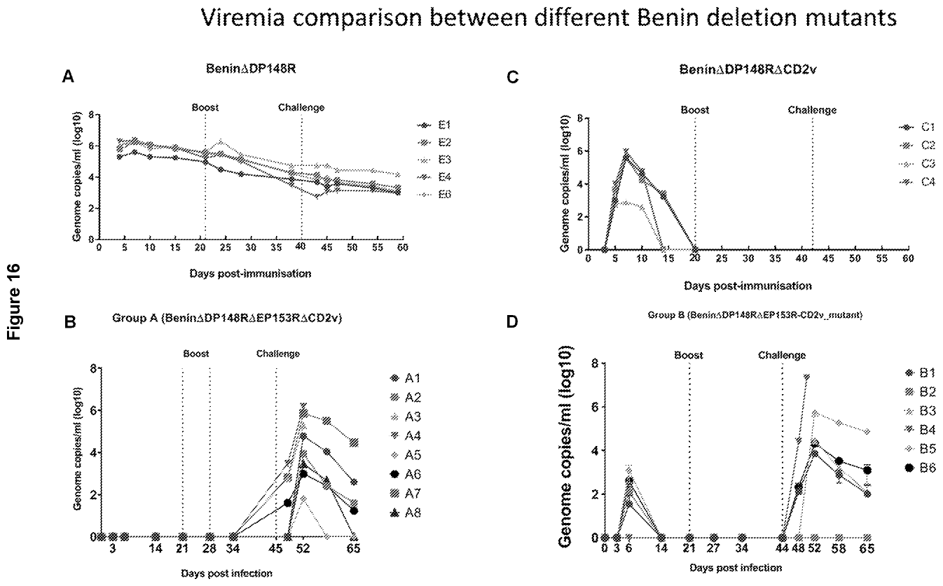

FIG. 16 directly compares virus genome in blood after immunisation with different viruses. Immunisation with BeninΔDP148R induces moderate levels of virus genome transiently, which decrease slowly over 60 days. No increase in replication or detectable challenge virus is observed (FIG. 16A). Deletion of the EP402R/CD2v gene (BeninΔDP148RΔCD2v virus) dramatically reduces the persistence of genome in blood (˜15 days maximum) and reduces the level in some pigs. No virus genome was detected post-challenge (FIG. 16B). The triple deletion BeninΔDP148RΔEP153RΔCD2v induces no detectable genome in blood post-immunisation and moderate to low levels after challenge decreasing to low or undetectable levels by the end of the experiment (FIG. 16C). The BeninΔDP148RΔEP153R-CD2vE99R virus induces a transient low level of virus genome in blood after immunisation and low levels after challenge (FIG. 16D).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in virology, molecular biology or related fields are intended to be within the scope of the following claims.

1. An attenuated African Swine Fever (ASF) virus in which the expression and/or activity of the genes DP148R, EP153R and EP402R is disrupted;
and which comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

2. An attenuated ASF virus according to claim 1 wherein the DP148R gene and/or the EP153R gene is at least partially deleted.

3. An attenuated ASF virus according to claim 2 wherein the DP148R gene and/or the EP153R gene is completely deleted.

4. An attenuated ASF virus according to claim 1 wherein the DP148R gene and/or the EP153R gene is interrupted.

5. An attenuated ASF virus according to any of claims 1 to 4 wherein the ability of the DP148R gene to inhibit NF-κB activity is disrupted.

6. An attenuated ASF virus according to any of claims 1 to 5 wherein the ability of the DP148R gene to inhibit translocation of the p65 sub-unit of NF-κB activity to the nucleus is disrupted.

5. An attenuated ASF virus according to any of claims 1 to 4 wherein the ability of the EP153R gene and/or the EP402R gene to mediate haemadsorption is disrupted.

6. An attenuated ASF virus according to any of claims 1 to 5 wherein the EP402R gene comprises one or more mutations that disrupt ligand binding by the EP402R protein.

7. An attenuated ASF virus according to claim 6 wherein the EP402R gene comprises one or more mutations that change one or more amino acids in the ligand-binding domain of the EP402R protein.

8. An attenuated ASF virus according to claim 7 wherein the one or more mutations change an amino acid at a position in the EP402R protein which corresponds to E99 and/or Y102 of Benin 97/1 EP402R protein (SEQ ID No. 11).

9. An attenuated ASF virus according to claim 8 wherein the amino acid at the position which corresponds to E99 is changed to R and/or the amino acid at the position equivalent to Y102 is changed to D.

10. An attenuated ASF virus according to any of claims 1 to 6 wherein the EP402R gene is at least partially deleted.

11. An attenuated ASF virus according to claim 10 wherein the EP402R gene is completely deleted.

12. An attenuated ASF virus according to any of claims 1 to 11 which comprises functional versions of all ASF virus genes other than DP148R, EP153R and EP402R.

13. A vaccine comprising an attenuated ASF virus according to any of claims 1 to 12.

14. A vaccine according to claim 13 for use in treating and/or preventing African Swine Fever in a subject.

15. A method for treating and/or preventing African Swine Fever in a subject which comprises the step of administering to the subject an effective amount of a vaccine according to claim 13.

16. A vaccine for use according to claim 14, or a method according to claim 15, wherein the subject is a domestic pig.

17. A vaccine for use according to claim 14 or 16, or a method according to claim 15 or 16, in which the vaccine is administered following a prime-boost regime.

18. A method of attenuating an ASF virus which comprises the step of disrupting the expression and/or activity of the following genes:
DP148R
EP153R and
EP402R.

19. A method according to claim 18 wherein the DP148R gene and/or the EP153R gene is at least partially deleted.

20. A method according to claim 19 wherein the DP148R gene and/or the EP153R gene is completely deleted.

21. A method according to claim 18 wherein the DP148R gene and/or the EP153R gene is interrupted.

22. A method according to any of claims 18 to 21 comprising disrupting the ability of the EP402R gene to mediate haemadsorption.

23. A method according to any of claims 18 to 22 comprising introducing one or more mutations in the EP402R gene that disrupt ligand binding by the EP402R protein.

24. A method according to claim 23 comprising introducing one or more mutations in the EP402R gene that change one or more amino acids in the ligand-binding domain of the EP402R protein.

25. A method according to claim 24 comprising changing the amino acid at position R96, E99 and/or Y102 of the EP402R protein.

26. A method according to claim 25 comprising changing the amino acid at position R96 to Q, the amino acid at position E99 to R and/or the amino acid at position Y102 to D.

27. A method according to any of claims 18 to 23 wherein the EP402R gene is at least partially deleted.

28. A method according to claim 28 wherein the EP402R gene is completely deleted.

29. A method according to claim 28 wherein the EP402R gene is interrupted.
